(19) United States
(12) Patent Application Publication
(10) Pub. No.: US 2020/0361895 A1
(43) Pub. Date: Nov. 19, 2020

(54) NOVEL SULFONAMIDE CARBOXAMIDE COMPOUNDS

(71) Applicant: Inflazome Limited, Dublin 2 (IE)

(72) Inventors: Matthew COOPER, Cambridge (GB); David MILLER, Cambridge (GB); Angus MACLEOD, Cambridge (GB); Jimmy VAN WILTENBURG, Groningen (NL); Stephen THOM, Nottingham (GB); Stephen ST-GALLAY, Nottingham (GB); Jonathan SHANNON, Nottingham (GB); Thomas ALANINE, Nottingham (GB); Stuart ONIONS, Nottingham (GB); Ian STRUTT, Nottingham (GB)

(73) Assignee: Inflazome Limited, Dublin 2 (IE)

(21) Appl. No.: 16/638,698

(22) PCT Filed: Aug. 15, 2018
(86) PCT No.: PCT/EP2018/072111

§ 371 Date: Feb. 12, 2020

(30) Foreign Application Priority Data
Aug. 15, 2017 (GB) 1713082.4
Nov. 9, 2017 (GB) 1718563.8
Dec. 22, 2017 (GB) 1721726.6
Jul. 4, 2018 (GB) 1810983.5

(51) Int. Cl. C07D 401/12; C07D 403/12; C07D 231/18; C07D 417/12; C07D 413/12; C07D 407/12; C07D 407/14; C07D 213/52; C07D 401/14; C07D 471/08; C07D 405/10; C07D 405/14; C07D 487/04

(52) U.S. Cl. CPC C07D 401/12; C07D 403/12; C07D 231/18; C07D 417/12; C07D 413/12; C07D 407/12; C07D 487/04; C07D 213/52; C07D 401/14; C07D 471/08; C07D 405/10; C07D 405/14; C07D 407/14

(57) ABSTRACT

The present invention relates to compounds of formula (I):

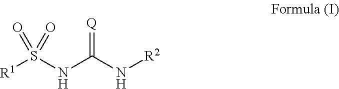

wherein Q is selected from O or S; R¹ is a saturated or unsaturated, optionally substituted hydrocarbyl group optionally including one or more heteroatoms N, O or S; and R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group may optionally be further substituted.

The present invention further relates to salts, solvates and prodrugs of such compounds, to pharmaceutical compositions comprising such compounds, and to the use of such compounds in the treatment and prevention of medical disorders and diseases, most especially by the inhibition of NLRP3.

### CROSS-REFERENCE TO RELATED APPLICATION

This application is the US national stage entry of PCT/EP2018/072111 filed Aug. 15, 2018, which claims priority to GB 1713082.4 filed Aug. 15, 2017; GB 1718563.8 filed Nov. 9, 2017; GB 1721726.6 filed Dec. 22, 2017; and GB 1810983.5 filed Jul. 4, 2018.

### FIELD OF THE INVENTION

The present invention relates to sulfonylureas and sulfonylthioureas comprising a cyclic group attached to the nitrogen atom of the urea group, wherein the cyclic group is substituted at the α-position with a monovalent heterocyclic group or a monovalent aromatic group, and to associated salts, solvates, prodrugs and pharmaceutical compositions. The present invention further relates to the use of such compounds in the treatment and prevention of medical disorders and diseases, most especially by NLRP3 inhibition.

### BACKGROUND

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β nd IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signaling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al.; J. Pharmacol. Exp. Ther. 299, 187-197, 2001). CRIDs are a class of diarylsulfonylurea-containing compounds that inhibit the post-translational processing of IL-1β. Post-translational processing of IL-1β is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59 (5), 1691-1710, 2016; and WO 2016/131098 A1, WO 2017/129897 A1, WO 2017/140778 A1, WO 2017/184604 A1, WO 2017/184623 A1, WO 2017/184624 A1, WO2018/015445 A1 and WO 2018/136890 A1).

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

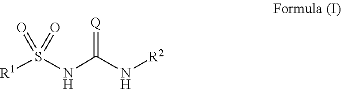

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and

R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group may optionally be further substituted.

In one embodiment, the compound is not:

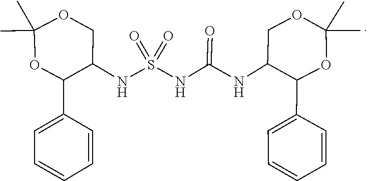

In one embodiment, the compound is not:

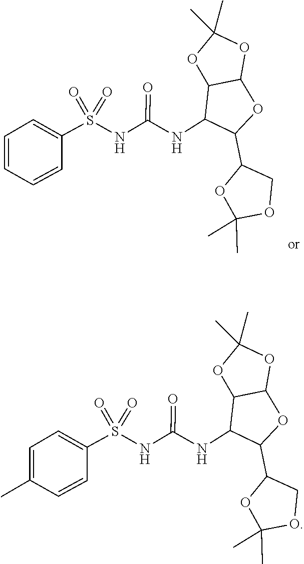 [SMILES: CC1(C)OCC(C2OC3OC(C)(C)OC3C2NC(=O)NS(=O)(=O)c2ccccc2)O1 ; Cc1ccc(S(=O)(=O)NC(=O)NC2C(C3COC(C)(C)O3)OC3OC(C)(C)OC32)cc1]

In one embodiment, the invention provides a compound of formula (I):

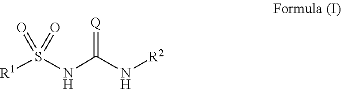

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group is further substituted at the α′ position and may optionally be further substituted.

In a further embodiment, the invention provides a compound of formula (I):

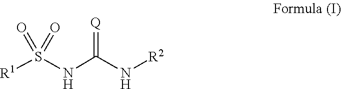

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group is further substituted at the α′ position and may optionally be further substituted;
provided that R¹ is not substituted or unsubstituted phenyl; and provided that the substituent at the α′ position of the cyclic group of R² is not —CN, —CH₃, —COOH or —COOEt.

In a further embodiment, the invention provides a compound of formula (I):

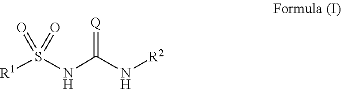

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group is further substituted at the α′ position and may optionally be further substituted;
provided that R¹ is not unsubstituted methyl, unsubstituted cyclopropyl, unsubstituted cyclohexyl, or substituted or unsubstituted phenyl; and provided that the substituent at the α′ position of the cyclic group of R² is not —CN.

In a further embodiment, the invention provides a compound of formula (I):

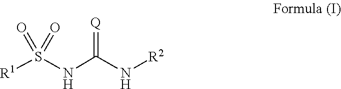

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group is further substituted at the α′ position and may optionally be further substituted;
provided that R¹ is not substituted or unsubstituted phenyl; and provided that the substituent at the α′ position of the cyclic group of R² is not —CN; and provided that the cyclic group of R² is not pyrazol-5-yl or isoxazol-4-yl.

In a further embodiment, the invention provides a compound of formula (I):

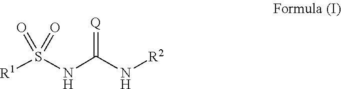

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group is further substituted at the α′ position and may optionally be further substituted;
provided that R¹ is not substituted or unsubstituted phenyl; and provided that the substituent at the α′ position of the cyclic group of R² is not —CN; and provided that the cyclic group of R² is not imidazol-5-yl or isoxazol-4-yl.

In a further embodiment, the invention provides a compound of formula (I):

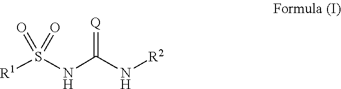

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a 5- or 6-membered cyclic group substituted at the α and α′ positions and at least one further position and optionally further substituted, wherein the substituent at the α-position is a monovalent heterocyclic group or a monovalent aromatic group, wherein a ring atom of the heterocyclic or aromatic group is directly attached to the α ring atom of the 5- or 6-membered cyclic group, wherein the heterocyclic or aromatic group may optionally be substituted, and provided that the 5- or 6-membered cyclic group is not pyrazol-5-yl, 1,2-dihydropyrazol-3-one-4-yl, tetrahydrofuran-3-yl, pyrrolidin-1-yl, 1,4-dihydropyridin-2-yl, 4H-1,2,4-triazin-5-one-4-yl, 3H-quinazolin-4-one-3-yl or 1,4-dioxido-quinoxalin-2-yl.

In a further embodiment, the invention provides a compound of formula (I):

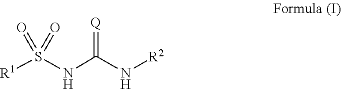

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a 5- or 6-membered cyclic group substituted at the α and α′ positions and optionally further substituted, wherein the substituent at the α-position is a monovalent heterocyclic group or a monovalent aromatic group, wherein a ring atom of the heterocyclic or aromatic group is directly attached to the α ring atom of the 5- or 6-membered cyclic group, wherein the heterocyclic or aromatic group may optionally be substituted, and provided that the 5- or 6-membered cyclic group is not pyrazol-5-yl, imidazol-5-yl, isoxazol-4-yl, 1,2-dihydropyrazol-3-one-4-yl, tetrahydrofuran-3-yl, pyrrolidin-1-yl, 1,4-dihydropyridin-2-yl, 4H-1,2,4-triazin-5-one-4-yl, 3H-quinazolin-4-one-3-yl or 1,4-dioxido-quinoxalin-2-yl.

In a further embodiment, the invention provides a compound of formula (I):

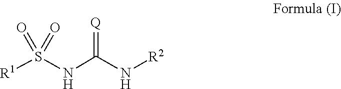

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a 6-membered cyclic group substituted at the 2- and 6-positions and optionally further substituted, wherein the substituent at the 2- or 6-position is a monovalent heterocyclic group or a monovalent aromatic group, wherein a ring atom of the heterocyclic or aromatic group is directly attached to a ring atom of the cyclic group, wherein the heterocyclic or aromatic group may optionally be substituted, and provided that the 6-membered cyclic group is not 1,4-dihydropyridin-2-yl, 4H-1,2,4-triazin-5-one-4-yl, 3H-quinazolin-4-one-3-yl or 1,4-dioxido-quinoxalin-2-yl.

In a further embodiment, the invention provides a compound of formula (I):

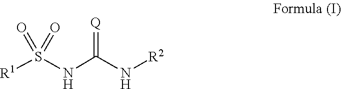

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is phenyl substituted at the 2- and 6-positions and optionally further substituted, wherein the substituent at the 2- or 6-position is a monovalent heterocyclic group or a monovalent aromatic group, wherein a ring atom of the heterocyclic or aromatic group is directly attached to a ring atom of the cyclic group, and wherein the heterocyclic or aromatic group may optionally be substituted.

In a further embodiment, the invention provides a compound of formula (I):

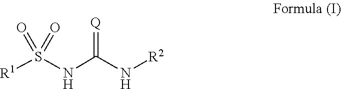

wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is phenyl substituted at the 2-, 4- and 6-positions and optionally further substituted, wherein the substituent at the 2- or 6-position is a monovalent heterocyclic group or a monovalent aromatic group, wherein a ring atom of the heterocyclic or aromatic group is directly attached to a ring atom of the cyclic group, and wherein the heterocyclic or aromatic group may optionally be substituted.

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₂ hydrocarbyl group.

More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

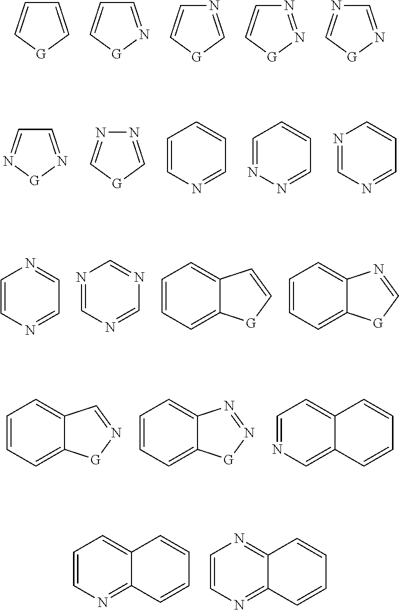

wherein G=0, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety:

(i) each hydrogen atom may optionally be replaced by a group independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(N₂)R^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂; and/or (ii) any two hydrogen atoms attached to the same atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N═N—, —N(R^{β})—, —N(O)(R^{β})—, —N⁺(R^{β})₂— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —COO(C₁-C₄ alkyl), —COO(C₁-C₄ haloalkyl), halo, —OH, —NH₂, —CN, —C≡CH, oxo (═O), or 4- to 6-membered heterocyclic group.

Typically, the compounds of the present invention comprise at most one quaternary ammonium group such as —N⁺(R^{β})₃ or —N⁺(R^{β})₂—.

Where reference is made to a —R^{α}—C(N₂)R^{β} group, what is intended is:

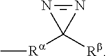

Typically, in an optionally substituted group or moiety:

(i) each hydrogen atom may optionally be replaced by a group independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —NH—R^{α}—OH; —NH—R^{α}—OR^{β}; —NH—R^{α}—NH₂; —NH—R^{α}—NHR^{β}; —NH—R^{α}—N(R^{β})₂; —NR^{β}—R^{α}—OH; —NR^{β}—R^{α}—OR^{β}; —NR^{β}—R^{α}—NH₂; —NR^{β}—R^{α}—NHR^{β}; or —NR^{β}—R^{α}—N(R^{β})₂; and/or
(ii) any two hydrogen atoms attached to the same carbon atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Typically, in an optionally substituted group or moiety:

(i) each hydrogen atom may optionally be replaced by a group independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β}; and/or
(ii) any two hydrogen atoms attached to the same carbon atom may optionally be replaced by a γ-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Typically, in an optionally substituted group or moiety:

(i) each hydrogen atom may optionally be replaced by a group independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; or —R^{α}—OCOR^{β}; and/or
(ii) any two hydrogen atoms attached to the same carbon atom may optionally be replaced by a π-bonded substituent independently selected from oxo (═O), ═S, ═NH or ═NR^{β}; and/or
(iii) any two hydrogen atoms attached to the same or different atoms, within the same optionally substituted group or moiety, may optionally be replaced by a bridging substituent independently selected from —O—, —S—, —NH—, —N(R^{β})— or —R^{α}—;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl or halo groups.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{β})—, —N(O)(R^{β})—, —N⁺(R^{β})₂— or —R^{α}—) of an optionally substituted group or moiety (e.g. R¹) must only be attached to the specified group or moiety and may not be attached to a second group or moiety (e.g. R²), even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

As used herein, the nomenclature α, β, α′, β′ refers to the position of the atoms of a cyclic group, such as —R², relative to the point of attachment of the cyclic group to the remainder of the molecule. For example, where the cyclic group is a phenyl moiety, the α, β, α′ and β′ positions are as follows:

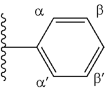 [SMILES: *c1ccccc1]

For the avoidance of doubt, where it is stated that a cyclic group is substituted at the α and/or α′ positions, it is to be understood that one or more hydrogen atoms at the α and/or α′ positions respectively are replaced by one or more substituents. Unless stated otherwise the term ‘substituted’ does not include the replacement of one or more ring carbon atoms by one or more ring heteroatoms.

Where reference is made to a hydrocarbyl or other group including one or more heteroatoms N, O or S in its carbon skeleton, or where reference is made to a carbon atom of a hydrocarbyl or other group being replaced by an N, O or S atom, what is intended is that:

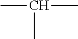

is replaced by

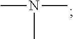

—CH₂— is replaced by —NH—, —O— or —S—;
—CH₃ is replaced by —NH₂, —OH or —SH;
CH═ is replaced by —N═;
CH₂═ is replaced by NH═, O═ or S═; or
CH≡ is replaced by N≡;
   provided that the resultant group comprises at least one carbon atom. For example, methoxy, dimethylamino and aminoethyl groups are considered to be hydrocarbyl groups including one or more heteroatoms N, O or S in their carbon skeleton.

Where reference is made to a —CH₂— group in the backbone of a hydrocarbyl or other group being replaced by a —N(O)(R^{β})— or —N⁺(R^{β})₂— group, what is intended is that:
—CH₂— is replaced by

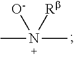

or
—CH₂— is replaced by

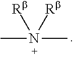

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are to be counted as carbon atoms when calculating the number of carbon atoms in a Cₓ-C_{y} group. For example, a morpholinyl group is to be considered a C₆ heterocyclic group, not a C₄ heterocyclic group.

For the purposes of the present specification, where it is stated that a first atom or group is “directly attached” to a second atom or group it is to be understood that the first atom or group is covalently bonded to the second atom or group with no intervening atom(s) or groups being present. So, for example, for the group (C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

R¹ is a saturated or unsaturated (including aromatic) hydrocarbyl group, such as a C₁-C₃₀ or C₂-C₂₀ or C₃-C₁₇ hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton.

In one embodiment, R¹ is a 4- to 10-membered cyclic group, wherein the cyclic group may optionally be substituted. Typically the cyclic group is a cycloalkyl, cycloalkenyl, non-aromatic heterocyclic, aryl or heteroaryl group. In one embodiment, R¹ is a phenyl, naphthyl, pyridinyl, pyridazinyl, pyrimidinyl, pyrazinyl, pyrrolyl, furanyl, thiophenyl, pyrazolyl, imidazolyl, triazolyl, oxazolyl, isoxazolyl, thiazolyl, isothiazolyl, cyclopropyl, cyclobutyl, cyclopentyl, cyclohexyl, cycloheptyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, 1,4-dioxanyl, thianyl, piperazinyl, morpholinyl, thiomorpholinyl, 2-oxo-1,2-dihydropyridinyl, 2-oxo-1,2-dihydropyrazinyl or 2-oxo-1,2-dihydropyrimidinyl group, all of which may optionally be substituted. In one embodiment, R¹ is a phenyl, naphthyl, pyridinyl, pyridazinyl, pyrimidinyl, pyrazinyl, pyrrolyl, furanyl, thiophenyl, pyrazolyl, imidazolyl, triazolyl, oxazolyl, isoxazolyl, thiazolyl, isothiazolyl, cyclopropyl, cyclobutyl, cyclopentyl, cyclohexyl, cycloheptyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, 1,4-dioxanyl or thianyl group, all of which may optionally be substituted. In one embodiment, R¹ is a phenyl, pyridinyl, pyridazinyl, pyrimidinyl, pyrazinyl, pyrazolyl, imidazolyl, triazolyl, oxazolyl, azetidinyl, pyrrolidinyl, piperidinyl, piperazinyl, 2-oxo-1,2-dihydropyridinyl, 2-oxo-1,2-dihydropyrazinyl or 2-oxo-1,2-dihydropyrimidinyl group, all of which may optionally be substituted. In one embodiment, R¹ is a pyrazolyl, imidazolyl, triazolyl, azetidinyl, pyrrolidinyl or piperidinyl group, all of which may optionally be substituted.

In another embodiment, R¹ is a C₁-C₁₅ alkyl, C₂-C₁₅ alkenyl or C₂-C₁₅ alkynyl group, all of which may optionally be substituted, and all of which may optionally include one or more (such as one, two or three) heteroatoms N, O or S in their carbon skeleton. R¹ may be a C₁-C₁₀ alkyl, C₂-C₁₀ alkenyl or C₂-C₁₀ alkynyl group, all of which may optionally be substituted, and all of which may optionally include one or more (such as one, two or three) heteroatoms N, O or S in their carbon skeleton. In one embodiment, R¹ is an optionally substituted C₁-C₅ alkyl or C₂-C₅ alkenyl group.

In another embodiment, R¹ is an optionally substituted phenyl or optionally substituted benzyl group.

In another embodiment, R¹ is a hydrocarbyl group, wherein the hydrocarbyl group may be straight-chained or branched, or be or include cyclic groups, wherein the hydrocarbyl group may optionally be substituted, and wherein the hydrocarbyl group includes one or more heteroatoms N or O in its carbon skeleton or is substituted with one or more heteroatoms N or O (i.e. substituted with a substituent comprising one or more heteroatoms N or O). Typically the hydrocarbyl group contains 1-15 carbon atoms and 1-4 nitrogen or oxygen atoms.

In the above embodiments, R¹ may be substituted with one or more substituents independently selected from halo; —CN; —NO₂; —N₃; —R^{β}; —OH; —OR^{β}; —R^{α}-halo; —R^{α}—CN; —R^{α}—NO₂; —R^{α}—N₃; —R^{α}—R^{β}; —R^{α}—OH; —R^{α}—OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —Si(R^{β})₃; —O—Si(R^{β})₃; —R^{α}—Si(R^{β})₃; —R^{α}—O—Si(R^{β})₃; —NH₂; —NHR^{β}; —N(R^{β})₂; —N(O)(R^{β})₂; —N⁺(R^{β})₃; —R^{α}—NH₂; —R^{α}—NHR^{β}; —R^{α}—N(R^{β})₂; —R^{α}—N(O)(R^{β})₂; —R^{α}—N⁺(R^{β})₃; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —C(═NH)R^{β}; —C(═NH)NH₂; —C(═NH)NHR^{β}; —C(═NH)N(R^{β})₂; —C(═NR^{β})R^{β}; —C(═NR^{β})NHR^{β}; —C(═NR^{β})N(R^{β})₂; —C(═NOH)R^{β}; —C(N₂)R^{β}; —R^{α}—C(═NH)R^{β}; —R^{α}—C(═NH)NH₂; —R^{α}—C(═NH)NHR^{β}; —R^{α}—C(═NH)N(R^{β})₂; —R^{α}—C(═NR^{β})R^{β}; —R^{α}—C(═NR^{β})NHR^{β}; —R^{α}—C(═NR^{β})N(R^{β})₂; —R^{α}—C(═NOH)R^{β}; —R^{α}—C(N₂)R^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —O—R^{α}—N(O)(R^{β})₂; —O—R^{α}—N⁺(R^{β})₃; —NH—R^{α}—OH; —NH—R^{α}—OR^{β}; —NH—R^{α}—NH₂; —NH—R^{α}—NHR^{β}; —NH—R^{α}—N(R^{β})₂; —NH—R^{α}—N(O)(R^{β})₂; —NH—R^{α}—N⁺(R^{β})₃; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; —NR^{β}—R^{α}—N(O)(R^{β})₂; —NR^{β}—R^{α}—N⁺(R^{β})₃; —N(O)R^{β}—R^{α}—OH; —N(O)R^{β}—R^{α}—OR^{β}; —N(O)R^{β}—R^{α}—NH₂; —N(O)R^{β}—R^{α}—NHR^{β}; —N(O)R^{β}—R^{α}—N(R^{β})₂; —N(O)R^{β}—R^{α}—N(O)(R^{β})₂; —N(O)R^{β}—R^{α}—N⁺(R^{β})₃; —N⁺(R^{β})₂—R^{α}—OH; —N⁺(R^{β})₂—R^{α}—OR^{β}; —N⁺(R^{β})₂—R^{α}—NH₂; —N⁺(R^{β})₂—R^{α}—NHR^{β}; —N⁺(R^{β})₂—R^{α}—N(R^{β})₂; or —N⁺(R^{β})₂—R^{α}—N(O)(R^{β})₂;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, wherein one or more —CH₂— groups in the backbone of the alkylene, alkenylene or alkynylene group may optionally be replaced by one or more —N(O)(R^{β})— or —N⁺(R^{β})₂— groups, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, or wherein any two or three —R^{β} attached to the same nitrogen atom may, together with the nitrogen atom to which they are attached, form a C₂-C₇ cyclic group, and wherein any —R^{β} may optionally be substituted with one or more C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —COO(C₁-C₄ alkyl), —COO(C₁-C₄ haloalkyl), halo, —OH, —NH₂, —CN, —C≡CH, oxo (═O), or 4- to 6-membered heterocyclic group.

Alternatively, R¹ may be substituted with one or more substituents independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; —O—R^{α}—OH; —O—R^{α}—OR^{β}; —O—R^{α}—NH₂; —O—R^{α}—NHR^{β}; —O—R^{α}—N(R^{β})₂; —NH—R^{α}—OH; —NH—R^{α}—OR^{β}; —NH—R^{α}—NH₂; —NH—R^{α}—NHR^{β}; —NH—R^{α}—N(R^{β})₂; —NR^{β}—R^{α}—OH; —NR^{β}—R^{α}—OR^{β}; —NR^{β}—R^{α}—NH₂; —NR^{β}—R^{α}—NHR^{β}; —NR^{β}—R^{α}—N(R^{β})₂; a C₃-C₇ cycloalkyl group optionally substituted with one or more C₁-C₃ alkyl or C₁-C₃ haloalkyl groups; a C₃-C₇ cycloalkenyl group optionally substituted with one or more C₁-C₃ alkyl or C₁-C₃ haloalkyl groups; a 3- to 7-membered non-aromatic heterocyclic group optionally substituted with one or more C₁-C₆ alkyl or C₁-C₃ haloalkyl groups; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively, R¹ may be substituted with one or more substituents independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —NH—CHO; —NR^{β}—CHO; —NH—COR^{β}; —NR^{β}—COR^{β}; —CONH₂; —CONHR^{β}; —CON(R^{β})₂; —R^{α}—NH—CHO; —R^{α}—NR^{β}—CHO; —R^{α}—NH—COR^{β}; —R^{α}—NR^{β}—COR^{β}; —R^{α}—CONH₂; —R^{α}—CONHR^{β}; —R^{α}—CON(R^{β})₂; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively, R¹ may be substituted with one or more substituents independently selected from halo; —CN; —NO₂; —N₃; —R^{β}; —OH; —OR^{β}; —SH; —SR^{β}; —SOR^{β}; —SO₂H; —SO₂R^{β}; —SO₂NH₂; —SO₂NHR^{β}; —SO₂N(R^{β})₂; —R^{α}—SH; —R^{α}—SR^{β}; —R^{α}—SOR^{β}; —R^{α}—SO₂H; —R^{α}—SO₂R^{β}; —R^{α}—SO₂NH₂; —R^{α}—SO₂NHR^{β}; —R^{α}—SO₂N(R^{β})₂; —NH₂; —NHR^{β}; —N(R^{β})₂; —R^{α}—NH₂; —R^{α}—NHR^{β}; —R^{α}—N(R^{β})₂; —CHO; —COR^{β}; —COOH; —COOR^{β}; —OCOR^{β}; —R^{α}—CHO; —R^{α}—COR^{β}; —R^{α}—COOH; —R^{α}—COOR^{β}; —R^{α}—OCOR^{β}; —CONH₂; —CONHR^{β}; —CON(R^{β})₂; oxo (═O); or a C₁-C₄ alkylene bridge;
wherein each —R^{α}— is independently selected from an alkylene, alkenylene or alkynylene group, wherein the alkylene, alkenylene or alkynylene group contains from 1 to 6 atoms in its backbone, wherein one or two carbon atoms in the backbone of the alkylene, alkenylene or alkynylene group may optionally be replaced by one or two heteroatoms N, O or S, and wherein the alkylene, alkenylene or alkynylene group may optionally be substituted with one or more halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one or more C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively still, R¹ may be substituted with one, two or three substituents independently selected from halo; —CN; —N₃; —R^{β}; —OH; —OR^{β}; —SO₂R^{β}; —NH₂; —NHR^{β}; —N(R^{β})₂; —R^{α}—NH₂; —R^{α}—NHR^{β}; —R^{α}—N(R^{β})₂; —COR^{β}; —COOR^{β}; —OCOR^{β}; —R^{α}—COR^{β}; —R^{α}—COOR^{β}; —R^{α}—OCOR^{β}; —CONH₂; —CONHR^{β}; —CON(R^{β})₂; or oxo (═O);
wherein each —R^{α}— is independently selected from a C₁-C₆ alkylene group, wherein one or two carbon atoms in the backbone of the alkylene group may optionally be replaced by one or two heteroatoms N, O or S, and wherein the alkylene group may optionally be substituted with one or two halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one, two or three C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

Alternatively still, R¹ may be substituted with one, two or three substituents independently selected from halo; C₁-C₅ alkyl; C₁-C₅ haloalkyl; —R⁵—(C₃-C₆ cycloalkyl); C₂-C₅ alkenyl; C₂-C₅ haloalkenyl; C₂-C₅ alkynyl; C₂-C₅ haloalkynyl; —R⁵—CN; —R⁵—N₃; —R⁵—NO₂; —R⁵—N(R⁶)₂; —R⁵—OR⁶; —R⁵—COR⁶; —R⁵—COOR⁶; —R⁵—CON(R⁶)₂; —R⁵—SO₂R⁶; —R⁵—(C₃-C₆ cycloalkyl substituted with —R⁵—N(R⁶)₂); —R⁵-phenyl; —R⁵-(Het); oxo (═O); or —R⁵¹—; wherein
R⁵ is independently selected from a bond or C₁-C₅ alkylene;
each R⁶ is independently selected from hydrogen; C₁-C₅ alkyl; C₁-C₅ haloalkyl; C₃-C₆ cycloalkyl; benzyl; or C₁-C₅ alkyl substituted with C₁-C₅ alkoxy; or two R⁶ together with the nitrogen atom to which they are attached may form a saturated 4- to 6-membered heterocyclic group;

R⁵¹ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted; and

Het is independently selected from a pyridinyl, 2-oxo-1,2-dihydropyridinyl, pyridazinyl, pyrimidinyl, pyrazinyl, pyrrolyl, pyrazolyl, imidazolyl, azetidinyl, pyrrolidinyl, piperidinyl, oxetanyl, tetrahydrofuranyl or tetrahydropyranyl group, each of which may optionally be substituted with one, two or three substituents independently selected from halo, C₁-C₄ alkyl, C₂-C₄ alkenyl, C₂-C₄ alkynyl or C₁-C₃ alkoxy.

Typically, any divalent group —R⁵¹— forms a 4- to 6-membered fused ring.

In one aspect of any of the above embodiments, R¹ contains from 1 to 3 atoms other than hydrogen. More typically, R¹ contains from 1 to 25 atoms other than hydrogen. More typically, R¹ contains from 2 to 20 atoms other than hydrogen. More typically, R¹ contains from 4 to 17 atoms other than hydrogen.

R² is a cyclic group substituted at the α-position with a monovalent heterocyclic group or a monovalent aromatic group, wherein a ring atom of the heterocyclic or aromatic group is directly attached to the α ring atom of the cyclic group, wherein the heterocyclic or aromatic group may optionally be substituted, and wherein the cyclic group may optionally be further substituted. For the avoidance of doubt, it is noted that it is a ring atom of the cyclic group of R² that is directly attached to the nitrogen atom of the urea or thiourea group, not any optional substituent.

In one embodiment, the α-substituted cyclic group of R² is a 5- or 6-membered cyclic group, wherein the cyclic group may optionally be further substituted. In one embodiment, the α-substituted cyclic group of R² is an aryl or a heteroaryl group, all optionally further substituted. In one embodiment, the α-substituted cyclic group of R² is a phenyl or a 5- or 6-membered heteroaryl group, all optionally further substituted. In one embodiment, the α-substituted cyclic group of R² is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl or isothiazolyl group, all optionally further substituted. In one embodiment, the α-substituted cyclic group of R² is a phenyl or pyrazolyl group, all optionally further substituted. In one embodiment, the α-substituted cyclic group of R² is a phenyl group, which is optionally further substituted.

R² is a cyclic group substituted at the α-position with a monovalent heterocyclic group or a monovalent aromatic group, wherein the heterocyclic or aromatic group may optionally be substituted. In one embodiment, the monovalent heterocyclic or aromatic group at the α-position is a phenyl or a 5- or 6-membered heterocyclic group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl, thiomorpholinyl or 1-methyl-2-oxo-1,2-dihydropyridinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl or thiomorpholinyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, 1,4-dioxanyl or thianyl group, all of which may optionally be substituted. In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, piperidinyl or tetrahydropyranyl group, all of which may optionally be substituted. In one embodiment, the monovalent heterocyclic or aromatic group at the α-position is a phenyl or a 5- or 6-membered heterocyclic group, all of which may optionally be substituted, and wherein the 5- or 6-membered heterocyclic group comprises at least one nitrogen ring atom and/or at least one oxygen ring atom. In one embodiment, the monovalent heterocyclic or aromatic group at the α-position is a phenyl, pyridinyl, pyridazinyl, pyrimidinyl, pyrazinyl, pyrrolyl, furanyl, pyrazolyl, imidazolyl, oxazolyl, isoxazolyl, thiazolyl, isothiazolyl, triazolyl, oxadiazolyl, azetinyl, azetidinyl, oxetanyl, pyrrolidinyl, tetrahydrofuranyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, morpholinyl, thiomorpholinyl or 1-methyl-2-oxo-1,2-dihydropyridinyl group, all of which may optionally be substituted. In one embodiment, the monovalent heterocyclic or aromatic group at the α-position is a phenyl, pyridinyl, pyridazinyl, pyrimidinyl, pyrazolyl, imidazolyl, isoxazolyl, thiazolyl, tetrahydropyranyl or 1-methyl-2-oxo-1,2-dihydropyridinyl group, all of which may optionally be substituted. In one embodiment, the monovalent heterocyclic or aromatic group at the α-position is a phenyl, pyridinyl, pyrimidinyl, pyrazolyl, imidazolyl, isoxazolyl, thiazolyl or tetrahydropyranyl group, all of which may optionally be substituted. In one embodiment, the monovalent heterocyclic or aromatic group at the α-position is a phenyl, pyridinyl, pyrimidinyl or pyrazolyl group, all of which may optionally be substituted. In one embodiment, the monovalent heterocyclic or aromatic group at the α-position is an unsubstituted phenyl, pyridinyl, pyrimidinyl or pyrazolyl group. In one embodiment, the monovalent heterocyclic group at the α-position is a pyridin-2-yl, pyridin-3-yl or pyridin-4-yl group, all of which may optionally be substituted. In one embodiment, the monovalent heterocyclic group at the α-position is an unsubstituted pyridin-3-yl group or an optionally substituted pyridin-4-yl group.

For any of these monovalent heterocyclic or aromatic groups at the α-position mentioned in the immediately preceding paragraph, the monovalent heterocyclic or aromatic group may optionally be substituted with one or two substituents independently selected from halo, —OH, —NH₂, —CN, —NO₂, —B⁴, —OB⁴, —NHB⁴, —N(B⁴)₂, —CONH₂, —CONHB⁴, —CON(B⁴)₂, —NHCOB⁴, —NB⁴COB⁴, or —B⁴⁴—;
wherein each B⁴ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁴ together with the nitrogen atom to which they are attached may form a 4- to 6-membered heterocyclic group containing one or two ring heteroatoms N and/or O, wherein any B⁴ may optionally be halo-substituted and/or substituted with one or two substituents independently selected from —OH, —NH₂, —OB⁴⁵, —NHB⁴⁵ or —N(B⁴⁵)₂;
wherein each B⁴⁴ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁴⁵, —NHB⁴⁵ or —N(B⁴⁵)₂; and wherein each B⁴⁵ is independently selected from a C₁-C₃ alkyl or C₁-C₃ haloalkyl group.

Typically, any divalent group —B⁴⁴— forms a 4- to 6-membered fused ring.

In one embodiment, the monovalent heterocyclic or aromatic group at the α-position is a phenyl, pyridinyl, pyrimidinyl or pyrazolyl group, all of which may optionally be substituted with one or two substituents independently selected from halo, —OH, —NH₂, —CN, C₁-C₃ alkyl or —O(C₁-C₃ alkyl). In one embodiment, the monovalent heterocyclic group at the α-position is a pyridin-2-yl, pyridin-3-yl or pyridin-4-yl group, all of which may optionally be substituted with one or two substituents independently selected from halo, —OH, —NH₂, —CN, C₁-C₃ alkyl or —O(C₁-C₃ alkyl). In one embodiment, the monovalent heterocyclic group at the α-position is an unsubstituted pyridin-3-yl group or a pyridin-4-yl group optionally substituted with one or two substituents independently selected from halo, —OH, —NH₂, —CN, C₁-C₃ alkyl or —O(C₁-C₃ alkyl). Alternatively, any of these monovalent phenyl or heterocyclic groups at the α-position may optionally be substituted with one or two substituents independently selected from halo, —OH, —NH₂, —CN, —NO₂, —B⁴, —OB⁴, —NHB⁴ or —N(B⁴)₂, wherein each B⁴ is independently selected from a C₁-C₄ alkyl, C₂-C₄ alkenyl or C₂-C₄ alkynyl group all of which may optionally be halo-substituted.

R² is a cyclic group substituted at the α-position with a monovalent heterocyclic group or a monovalent aromatic group, wherein the cyclic group may optionally be further substituted. In one embodiment, the α-substituted cyclic group of R² is substituted at the α and α′ positions, and may optionally be further substituted. For example, the α-substituted cyclic group of R² may be a phenyl or a 6-membered heterocyclic group substituted at the 2- and 6-positions, or substituted at the 2-, 4- and 6-positions. In one embodiment, the α-substituted cyclic group of R² may be a phenyl group substituted at the 2- and 6-positions, or substituted at the 2-, 4- and 6-positions.

Where the α-substituted cyclic group of R² is a phenyl or a 6-membered heterocyclic group which is substituted at the 4-position and is optionally further substituted, typically the substituent in the 4-position is selected from a halo, —CN, C₁-C₃ alkyl or C₃-C₆ cycloalkyl group. In one embodiment, the substituent in the 4-position is selected from a fluoro, chloro, —CN or cyclopropyl group.

R² is a cyclic group substituted at the α-position with a monovalent heterocyclic group or a monovalent aromatic group, wherein the cyclic group may optionally be further substituted. In one embodiment, such further substituents are in the α′ position of the α-substituted cyclic group of R². Such further substituents may be independently selected from halo, —R^{δ}, —OR^{δ} or —COR^{δ} groups, wherein each R^{δ} is independently selected from a C₁-C₆ alkyl, C₂-C₆ alkenyl, C₂-C₆ alkynyl or C₂-C₆ cyclic group and wherein each R^{δ} is optionally further substituted with one or more halo groups. Typically, such further substituents on the α-substituted cyclic group of R² are independently selected from halo, C₁-C₆ alkyl (in particular C₃-C₆ branched alkyl) or C₃-C₆ cycloalkyl groups, e.g. fluoro, chloro, isopropyl, cyclopropyl, cyclohexyl or t-butyl groups, wherein the alkyl and cycloalkyl groups are optionally further substituted with one or more fluoro and/or chloro groups.

In one embodiment, —R² has a formula selected from:

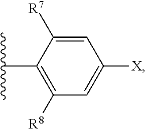

wherein R⁷ is C₁-C₄ alkyl, C₁-C₄ haloalkyl, C₃-C₆ cycloalkyl or C₃-C₆ halocycloalkyl, R⁸ is a 5- or 6-membered, optionally substituted heterocyclic or aromatic group, and X is hydrogen, halo, —OH, —NO₂, —CN, —R^{x}, —OR^{x}, —COR^{x}, —COOR^{x}, —CONH₂, —CONHR^{x} or —CON(R^{x})₂, wherein each —R^{x} is independently selected from C₁-C₄ alkyl, C₁-C₄ haloalkyl, C₃-C₄ cycloalkyl and C₃-C₄ halocycloalkyl. In one embodiment, the optional substituents on the heterocyclic or aromatic group are independently selected from halo, —OH, —NH₂, —CN, —NO₂, —B⁵, —OB⁵, —NHB⁵, —N(B⁵)₂, —CONH₂, —CONHB⁵, —CON(B⁵)₂, —NHCOB⁵, —NB⁵COB⁵, or —B⁵⁵—;
wherein each B⁵ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁵ together with the nitrogen atom to which they are attached may form a 4- to 6-membered heterocyclic group containing one or two ring heteroatoms N and/or O, wherein any B⁵ may optionally be halo-substituted and/or substituted with one or two substituents independently selected from —OH, —NH₂, —OB⁵⁶, —NHB⁵⁶ or —N(B⁵⁶)₂;
wherein each B⁵⁵ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁵⁶, —NHB⁵⁶ or —N(B⁵⁶)₂; and
wherein each B⁵⁶ is independently selected from a C₁-C₃ alkyl or C₁-C₃ haloalkyl group.

Typically, any divalent group —B⁵⁵— forms a 4- to 6-membered fused ring. Typically, R⁷ is C₁-C₄ alkyl or C₃-C₆ cycloalkyl, R⁸ is a 5- or 6-membered, optionally substituted heterocyclic or aromatic group, and X is hydrogen, halo, —CN, C₁-C₃ alkyl or C₃-C₆ cycloalkyl. More typically, R⁷ is C₁-C₄ alkyl, R⁸ is a 5- or 6-membered, optionally substituted heterocyclic or aromatic group, and X is hydrogen or halo. In one embodiment, the optional substituents on the heterocyclic or aromatic group are independently selected from halo, —OH, —NH₂, —CN, —NO₂, —B⁵, —OB⁵, —NHB⁵ or —N(B⁵)₂, wherein each B⁵ is independently selected from a C₁-C₄ alkyl, C₂-C₄ alkenyl or C₂-C₄ alkynyl group all of which may optionally be halo-substituted.

Typically, —R² has a formula selected from:

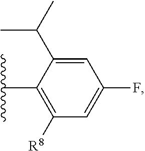

wherein R⁸ is a 5- or 6-membered, optionally substituted heterocyclic or aromatic group. In one embodiment, the optional substituents on the heterocyclic or aromatic group are independently selected from halo, —OH, —NH₂, —CN, —NO₂, —B⁶, —OB⁶, —NHB⁶, —N(B⁶)₂, —CONH₂, —CONHB⁶, —CON(B⁶)₂, —NHCOB⁶, —NB⁶COB⁶, or —B⁶⁶—;
wherein each B⁶ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁶ together with the nitrogen atom to which they are attached may form a 4- to 6-membered heterocyclic group containing one or two ring heteroatoms N and/or O, wherein any B⁶ may optionally be halo-substituted and/or substituted with one or two substituents independently selected from —OH, —NH₂, —OB⁶⁷, —NHB⁶⁷ or —N(B⁶⁷)₂;
wherein each B⁶⁶ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁶⁷, —NHB⁶⁷ or —N(B⁶⁷)₂; and
wherein each B⁶⁷ is independently selected from a C₁-C₃ alkyl or C₁-C₃ haloalkyl group.

Typically, any divalent group —B⁶⁶— forms a 4- to 6-membered fused ring. Typically, the optional substituents on the heterocyclic or aromatic group are independently selected from halo, —OH, —NH₂, —CN, —NO₂, —B⁶, —OB⁶, —NHB⁶ or —N(B⁶)₂, wherein each B⁶ is independently selected from a C₁-C₄ alkyl, C₂-C₄ alkenyl or C₂-C₄ alkynyl group all of which may optionally be halo-substituted.

The further substituents on the α-substituted cyclic group of R² also include cycloalkyl, cycloalkenyl, non-aromatic heterocyclic, aryl or heteroaryl rings which are fused to the α-substituted cyclic group of R². Typically, the cycloalkyl, cycloalkenyl, non-aromatic heterocyclic, aryl or heteroaryl rings are ortho-fused to the α-substituted cyclic group of R², i.e. each fused cycloalkyl, cycloalkenyl, non-aromatic heterocyclic, aryl or heteroaryl ring has only two atoms and one bond in common with the α-substituted cyclic group of R². Typically, the cycloalkyl, cycloalkenyl, non-aromatic heterocyclic, aryl or heteroaryl rings are ortho-fused to the α-substituted cyclic group of R² across the α′,β′ positions.

In one embodiment, —R² has a formula selected from:

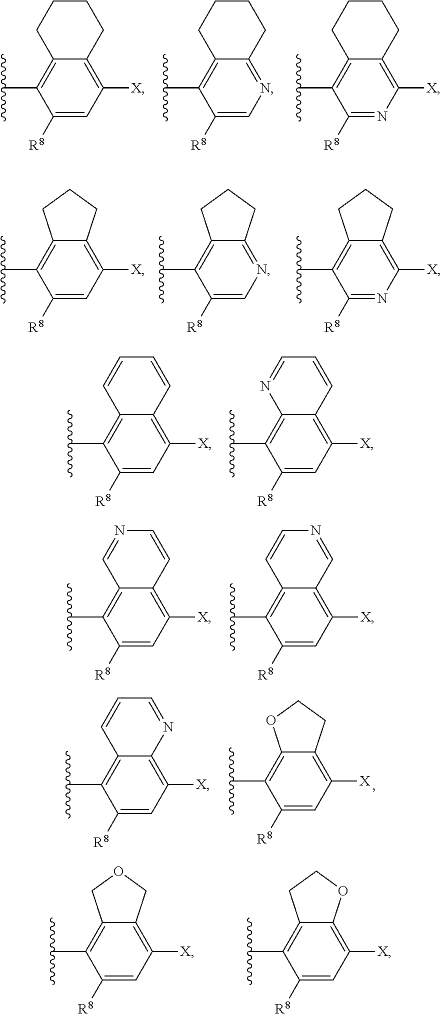

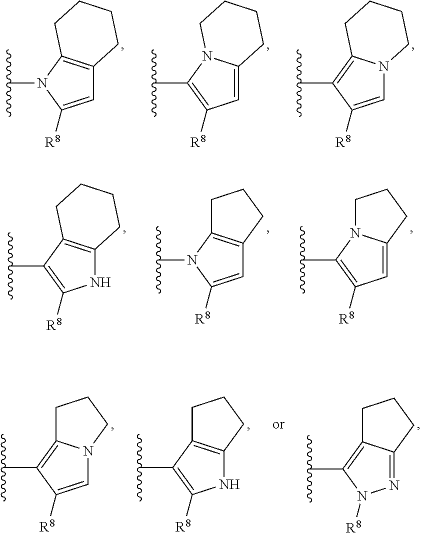

wherein R⁸ is a 5- or 6-membered, optionally substituted heterocyclic or aromatic group, and X is hydrogen, halo, —OH, —NO₂, —CN, —R^{x}, —OR^{x}, —COR^{x}, —COOR^{x}, —CONH₂, —CONHR^{x} or —CON(R^{x})₂, wherein each —R^{x} is independently selected from C₁-C₄ alkyl, C₁-C₄ haloalkyl, C₃-C₄ cycloalkyl and C₃-C₄ halocycloalkyl. In one embodiment, the optional substituents on the heterocyclic or aromatic group are independently selected from halo, —OH, —NH₂, —CN, —NO₂, —B⁷, —OB⁷, —NHB⁷, —N(B⁷)₂, —CONH₂, —CONHB⁷, —CON(B⁷)₂, —NHCOB⁷, —NB⁷COB⁷, or —B⁷⁷—;
wherein each B⁷ is independently selected from a C₁-C₄ alkyl, C₂-C₄ alkenyl, C₂-C₄ alkynyl, C₃-C₄ cycloalkyl or phenyl group, or a 4- to 6-membered heterocyclic group containing one or two ring heteroatoms N and/or O, or two B⁷ together with the nitrogen atom to which they are attached may form a 4- to 6-membered heterocyclic group containing one or two ring heteroatoms N and/or O, wherein any B⁷ may optionally be halo-substituted and/or substituted with one or two substituents independently selected from —OH, —NH₂, —OB⁷⁸, —NHB⁷⁸ or —N(B⁷⁸)₂;
wherein each B⁷⁷ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁷⁸, —NHB⁷⁸ or —N(B⁷⁸)₂; and
wherein each B⁷⁸ is independently selected from a C₁-C₃ alkyl or C₁-C₃ haloalkyl group.

Typically, any divalent group —B⁷⁷— forms a 4- to 6-membered fused ring. Typically, X is hydrogen, halo, —CN, C₁-C₃ alkyl, C₁-C₃ haloalkyl, cyclopropyl or halocyclopropyl. Typically, X is hydrogen, halo, —CN, C₁-C₃ alkyl or C₃-C₆ cycloalkyl. More typically, X is hydrogen or halo. Typically, the optional substituents on the heterocyclic or aromatic group are independently selected from halo, —OH, —NH₂, —CN, —NO₂, —B⁷, —OB⁷, —NHB⁷ or —N(B⁷)₂, wherein each B⁷ is independently selected from a C₁-C₄ alkyl, C₂-C₄ alkenyl or C₂-C₄ alkynyl group all of which may optionally be halo-substituted.

In one embodiment, —R² has a formula selected from:

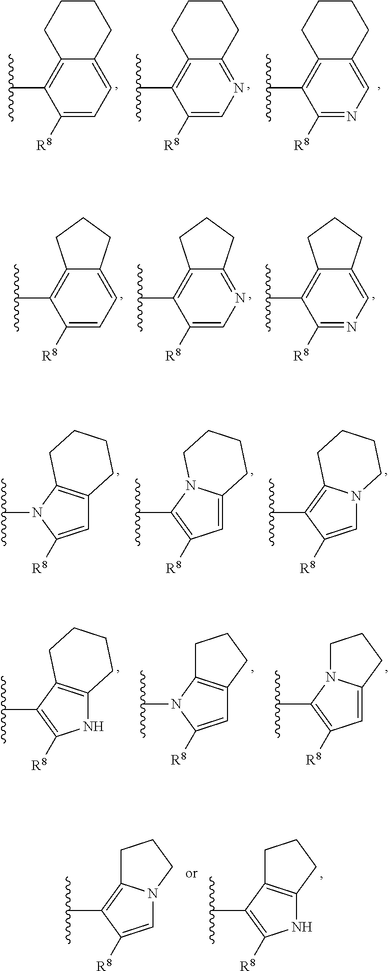

wherein R⁸ is a 5- or 6-membered, optionally substituted heterocyclic or aromatic group. In one embodiment, the optional substituents on the heterocyclic or aromatic group are independently selected from halo, —OH, —NH₂, —CN, —NO₂, —B⁸, —OB⁸, —NHB⁸, —N(B⁸)₂, —CONH₂, —CONHB⁸, —CON(B⁸)₂, —NHCOB⁸, —NB⁸COB⁸, or —B⁸⁸—;
wherein each B⁸ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁸ together with the nitrogen atom to which they are attached may form a 4- to 6-membered heterocyclic group containing one or two ring heteroatoms N and/or O, wherein any B⁸ may optionally be halo-substituted and/or substituted with one or two substituents independently selected from —OH, —NH₂, —OB⁸⁹, —NHB⁸⁹ or —N(B⁸⁹)₂;
wherein each B⁸⁸ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁸⁹, —NHB⁸⁹ or —N(B⁸⁹)₂; and
wherein each B⁸⁹ is independently selected from a C₁-C₃ alkyl or C₁-C₃ haloalkyl group.

Typically, any divalent group —B⁸⁸— forms a 4- to 6-membered fused ring. Typically, the optional substituents on the heterocyclic or aromatic group are independently selected from halo, —OH, —NH₂, —CN, —NO₂, —B⁸, —OB⁸, —NHB⁸ or —N(B⁸)₂, wherein each B⁸ is independently selected from a C₁-C₄ alkyl, C₂-C₄ alkenyl or C₂-C₄ alkynyl group all of which may optionally be halo-substituted.

Typically, —R² has a formula selected from:

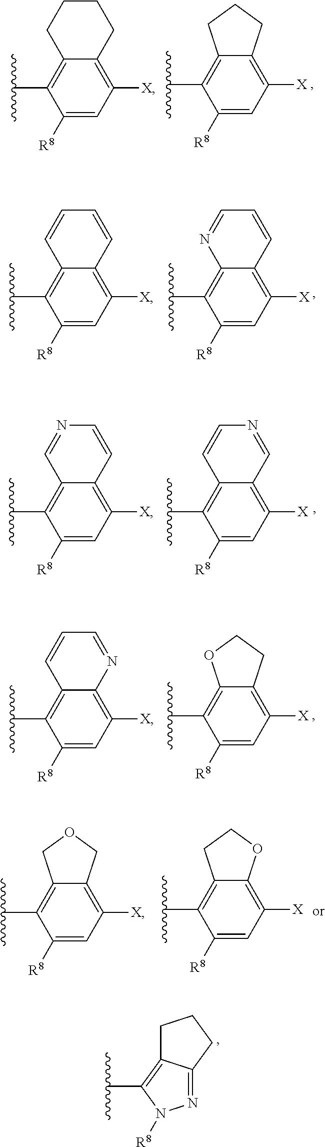

wherein R⁸ is a 5- or 6-membered, optionally substituted heterocyclic or aromatic group, and X is hydrogen, halo, —OH, —NO₂, —CN, —R^{x}, —OR^{x}, —COR^{x}, —COOR^{x}, —CONH₂, —CONHR^{x} or —CON(R^{x})₂, wherein each —R^{x} is independently selected from C₁-C₄ alkyl, C₁-C₄ haloalkyl, C₃-C₄ cycloalkyl and C₃-C₄ halocycloalkyl. In one embodiment, the optional substituents on the heterocyclic or aromatic group are independently selected from halo, —OH, —NH₂, —CN, —NO₂, —B⁹, —OB⁹, —NHB⁹, —N(B⁹)₂, —CONH₂, —CONHB⁹, —CON(B⁹)₂, —NHCOB⁹, —NB⁹COB⁹, or —B⁹⁹—;
wherein each B⁹ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁹ together with the nitrogen atom to which they are attached may form a 4- to 6-membered heterocyclic group containing one or two ring heteroatoms N and/or O, wherein any B⁹ may optionally be halo-substituted and/or substituted with one or two substituents independently selected from —OH, —NH₂, —OB⁹⁸, —NHB⁹⁸ or —N(B⁹⁸)₂;
wherein each B⁹⁹ is independently selected from a C₁-C₈ alkylene or C₂-C⁸ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁹⁸, —NHB⁹⁸ or —N(B⁹⁸)₂; and
wherein each B⁹⁸ is independently selected from a C₁-C₃ alkyl or C₁-C₃ haloalkyl group.

Typically, any divalent group —B⁹⁹— forms a 4- to 6-membered fused ring. Typically, X is hydrogen, halo, —CN, C₁-C₃ alkyl, C₁-C₃ haloalkyl, cyclopropyl or halocyclopropyl. Typically, X is hydrogen, halo, —CN, C₁-C₃ alkyl or C₃-C₆ cycloalkyl. More typically, X is hydrogen or halo. Typically, the optional substituents on the heterocyclic or aromatic group are independently selected from halo, —OH, —NH₂, —CN, —NO₂, —B⁹, —OB⁹, —NHB⁹ or —N(B⁹)₂, wherein each B⁹ is independently selected from a C₁-C₄ alkyl, C₂-C₄ alkenyl or C₂-C₄ alkynyl group all of which may optionally be halo-substituted.

In one aspect of any of the above embodiments, R² contains from 10 to 5 atoms other than hydrogen. More typically, R² contains from 10 to 4 atoms other than hydrogen. More typically, R² contains from 10 to 35 atoms other than hydrogen. Most typically, R² contains from 12 to 3 atoms other than hydrogen.

Q is selected from O or S. In one embodiment of the first aspect of the invention, Q is P.

In one specific embodiment, the invention provides a compound of formula (I), wherein:
Q is O;
R¹ is a saturated or unsaturated, optionally substituted, 4-, 5- or 6-membered heterocycle; or R¹ is an optionally substituted group selected from C₁-C₅ alkyl, C₂-C₅ alkenyl, C₂-C₅ alkynyl, C₃-C₆ cycloalkyl, phenyl or benzyl; or R¹ is a hydrocarbyl group, wherein the hydrocarbyl group may be straight-chained or branched, or be or include cyclic groups, wherein the hydrocarbyl group may optionally be substituted, and wherein the hydrocarbyl group includes one or more heteroatoms N or O in its carbon skeleton or is substituted with a substituent comprising one or more heteroatoms N or O (typically the hydrocarbyl group contains 1-15 carbon atoms and 1-4 nitrogen or oxygen atoms); and
R² is phenyl or a 5- or 6-membered heteroaryl group;
wherein the phenyl or 5- or 6-membered heteroaryl group is substituted at the α-position with a monovalent heterocyclic group or a monovalent aromatic group selected from a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl, thiomorpholinyl or 1-methyl-2-oxo-1,2-dihydropyridinyl group, wherein a ring atom of the heterocyclic or aromatic group is directly attached to the α ring atom of the phenyl or 5- or 6-membered heteroaryl group, and wherein the heterocyclic or aromatic group may optionally be substituted with one or two substituents independently selected from halo, —OH, —NH₂, —CN, —NO₂, —B⁴, —OB⁴, —NHB⁴, —N(B⁴)₂, —CONH₂, —CONHB⁴, —CON(B⁴)₂, —NHCOB⁴, —NB⁴COB⁴, or —B⁴⁴—;
wherein the phenyl or 5- or 6-membered heteroaryl group is either substituted at the α′ position with a C₁-C₅ alkyl, C₃-C₆ cycloalkyl, C₂-C₅ alkenyl, C₂-C₅ alkynyl or C₂-C₆ cyclic (typically a pyridinyl) group, or at the α′ and β′ positions with a divalent group —B⁴⁴—; and
wherein the phenyl or 5- or 6-membered heteroaryl group may optionally be further substituted (typically with one or two substituents independently selected from halo, —CN, C₁-C₃ alkyl or C₃-C₆ cycloalkyl);
wherein each B⁴ is independently selected from a C₁-C₄ alkyl, C₂-C₄ alkenyl, C₂-C₄ alkynyl, C₃-C₆ cycloalkyl or phenyl group, or a 4- to 6-membered heterocyclic group containing one or two ring heteroatoms N and/or O, or two B⁴ together with the nitrogen atom to which they are attached may form a 4- to 6-membered heterocyclic group containing one or two ring heteroatoms N and/or O, wherein any B⁴ may optionally be halo-substituted and/or substituted with one or two substituents independently selected from —OH, —NH₂, —OB⁴⁵, —NHB⁴⁵ or —N(B⁴⁵)₂;
wherein each B⁴⁴ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted and/or substituted with one or two substituents independently selected from —OH, —NH₂, —OB⁴⁵, —NHB⁴⁵ or —N(B⁴⁵)₂; and
wherein each B⁴⁵ is independently selected from a C₁-C₃ alkyl or C₁-C₃ haloalkyl group.

Typically, any divalent group —B⁴⁴— forms a 4- to 6-membered fused ring.

Typically, in this specific embodiment, the invention provides a compound of formula (I), wherein:
Q is O;
R¹ is a saturated or unsaturated, optionally substituted, 4-, 5- or 6-membered heterocycle; or R¹ is an optionally substituted group selected from C₁-C₅ alkyl, C₂-C₅ alkenyl, C₂-C₅ alkynyl, C₃-C₆ cycloalkyl, phenyl or benzyl; or R¹ is a hydrocarbyl group, wherein the hydrocarbyl group may be straight-chained or branched, or be or include cyclic groups, wherein the hydrocarbyl group may optionally be substituted, and wherein the hydrocarbyl group includes one or more heteroatoms N or O in its carbon skeleton or is substituted with a substituent comprising one or more heteroatoms N or O (typically the hydrocarbyl group contains 1-15 carbon atoms and 1-4 nitrogen or oxygen atoms); and
R² is phenyl or a 5- or 6-membered heteroaryl group;
wherein the phenyl or 5- or 6-membered heteroaryl group is substituted at the α-position with a monovalent heterocyclic group or a monovalent aromatic group selected from a phenyl, pyridinyl, pyrimidinyl, pyrazolyl, imidazolyl, triazolyl or tetrahydropyranyl group, wherein the heterocyclic or aromatic group may optionally be substituted with one or two substituents independently selected from halo, C₁-C₃ alkyl, C₁-C₃ haloalkyl, —R³—OR⁴, —R³—N(R⁴)₂, —R³—CN or —R³—C≡CR⁴, and wherein a ring atom of the heterocyclic or aromatic group is directly attached to the α ring atom of the phenyl or 5- or 6-membered heteroaryl group;
wherein the phenyl or 5- or 6-membered heteroaryl group is either substituted at the α′ position with a C₁-C₅ alkyl, C₃-C₆ cycloalkyl, C₂-C₅ alkenyl or C₂-C₅ alkynyl group, or at the α′ and β′ positions with a bridging C₂-C₅ alkylene or C₂-C₅ alkenylene group; and
wherein the phenyl or 5- or 6-membered heteroaryl group may optionally be further substituted (typically with one or two substituents independently selected from halo or —CN);

R³ is independently selected from a bond or C₁-C₃ alkylene; and R⁴ is independently selected from hydrogen or C₁-C₃ alkyl.

In this specific embodiment, R¹ may be an optionally substituted heterocycle selected from a pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, thianyl, piperazinyl, 1,4-dioxanyl, morpholinyl, thiomorpholinyl, 2-oxo-1,2-dihydropyridinyl, 2-oxo-1,2-dihydropyrazinyl or 2-oxo-1,2-dihydropyrimidinyl group. Alternatively, R¹ may be an optionally substituted heterocycle selected from a pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, thianyl, piperazinyl, 1,4-dioxanyl, morpholinyl or thiomorpholinyl group. Alternatively still, R¹ may be an optionally substituted heterocycle selected from a pyridinyl, pyridazinyl, pyrimidinyl, pyrazinyl, pyrazolyl, imidazolyl, triazolyl, oxazolyl, azetidinyl, pyrrolidinyl, piperidinyl, piperazinyl, 2-oxo-1,2-dihydropyridinyl, 2-oxo-1,2-dihydropyrazinyl or 2-oxo-1,2-dihydropyrimidinyl group.

Alternatively, in this specific embodiment, R¹ may be a C₁-C₅ alkyl or C₂-C₅ alkenyl group optionally substituted with one or two substituents independently selected from a halo, —CN, —N(R⁹)₂, —OR⁹, phenyl or heterocyclic group; wherein
each R⁹ is independently selected from hydrogen, C₁-C₅ alkyl or benzyl; and
the heterocyclic group is independently selected from a pyridinyl, azetidinyl, pyrrolidinyl, piperidinyl, oxetanyl, tetrahydrofuranyl or tetrahydropyranyl group, each of which may optionally be substituted with one or two substituents independently selected from halo or C₁-C₃ alkyl.

Alternatively, in this specific embodiment, R¹ may be a phenyl group optionally substituted with one or two substituents independently selected from C₁-C₅ alkyl, C₃-C₆ cycloalkyl, —R¹⁰—N(R¹¹)₂ or —R¹⁰—CON(R¹¹)₂; wherein R¹⁰ is independently selected from a bond or C₁-C₃ alkylene; and each R¹¹ is independently selected from hydrogen or C₁-C₃ alkyl.

Alternatively, in this specific embodiment, R¹ may be an unsubstituted benzyl group.

Alternatively, in this specific embodiment, R¹ may be a —OR¹², —NHR¹² or —N(R¹²)₂ group; wherein
each R¹² is independently selected from C₁-C₅ alkyl, C₃-C₆ cycloalkyl or —R¹³-(Het);
R¹³ is independently selected from a bond or C₁-C₃ alkylene; and
Het is independently selected from an azetidinyl, pyrrolidinyl, piperidinyl, oxetanyl, tetrahydrofuranyl or tetrahydropyranyl group, each of which may optionally be substituted with one or two substituents independently selected from halo or C₁-C₃ alkyl.

In this specific embodiment, R¹ may be optionally substituted with one, two or three substituents independently selected from halo; C₁-C₅ alkyl; C₁-C₅ haloalkyl; —R⁵—(C₃-C₆ cycloalkyl); C₂-C₅ alkenyl; C₂-C₅ haloalkenyl; C₂-C₅ alkynyl; C₂-C₅ haloalkynyl; —R⁵—CN; —R⁵—N₃; —R⁵—NO₂; —R⁵—N(R⁶)₂; —R⁵—OR⁶; —R⁵—COR⁶; —R⁵—COOR⁶; —R⁵—CON(R⁶)₂; —R⁵—SO₂R⁶; —R⁵—(C₃-C₆ cycloalkyl substituted with —R⁵—N(R⁶)₂); —R⁵-phenyl; —R⁵-(Het); oxo (═O); or —R⁵¹—; wherein
R⁵ is independently selected from a bond or C₁-C₅ alkylene;
each R⁶ is independently selected from hydrogen; C₁-C₅ alkyl; C₁-C₅ haloalkyl; C₃-C₆ cycloalkyl; benzyl; or C₁-C₅ alkyl substituted with C₁-C₅ alkoxy; or two R⁶ together with the nitrogen atom to which they are attached may form a saturated 4- to 6-membered heterocyclic group;
R⁵¹ is independently selected from a C₁-C₈ alkylene or C₂-C₈ alkenylene group, wherein one or two carbon atoms in the backbone of the alkylene or alkenylene group may optionally be replaced by one or two heteroatoms N and/or O, and wherein the alkylene or alkenylene group may optionally be halo-substituted; and
Het is independently selected from a pyridinyl, 2-oxo-1,2-dihydropyridinyl, pyridazinyl, pyrimidinyl, pyrazinyl, pyrrolyl, pyrazolyl, imidazolyl, azetidinyl, pyrrolidinyl, piperidinyl, oxetanyl, tetrahydrofuranyl or tetrahydropyranyl group, each of which may optionally be substituted with one, two or three substituents independently selected from halo, C₁-C₄ alkyl, C₂-C₄ alkenyl, C₂-C₄ alkynyl or C₁-C₃ alkoxy.

Typically, any divalent group —R⁵¹— forms a 4- to 6-membered fused ring.

Alternatively, in this specific embodiment, R¹ may be optionally substituted with one, two or three substituents independently selected from halo, C₁-C₅ alkyl, C₁-C₅ haloalkyl, C₃-C₆ cycloalkyl, C₂-C₅ alkenyl, C₂-C₅ haloalkenyl, C₂-C₅ alkynyl, C₂-C₅ haloalkynyl, —R⁵—CN, —R⁵—N₃, —R⁵—NO₂, —R⁵—N(R⁶)₂, —R⁵—OR⁶, —R⁵—COR⁶, —R⁵—COOR⁶, —R⁵—CON(R⁶)₂, —R⁵—SO₂R⁶, oxo (═O),

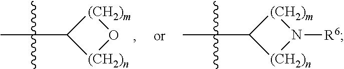

wherein
R⁵ is independently selected from a bond or C₁-C₃ alkylene;
each R⁶ is independently selected from hydrogen, C₁-C₅ alkyl, C₁-C₅ haloalkyl or C₃-C₆ cycloalkyl;
m is 1, 2 or 3; and
n is 1, 2 or 3.

In one aspect of any of the above embodiments, the compound of formula (I) has a molecular weight of from 250 to 2,000 Da. Typically, the compound of formula (I) has a molecular weight of from 300 to 1,000 Da. Typically, the compound of formula (I) has a molecular weight of from 340 to 800 Da. More typically, the compound of formula (I) has a molecular weight of from 380 to 600 Da.

A second aspect of the invention provides a compound selected from the group consisting of:

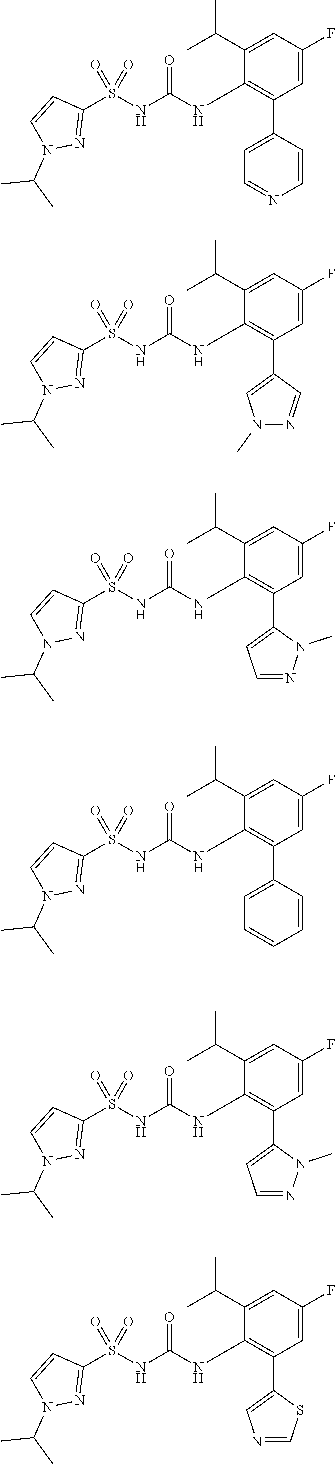

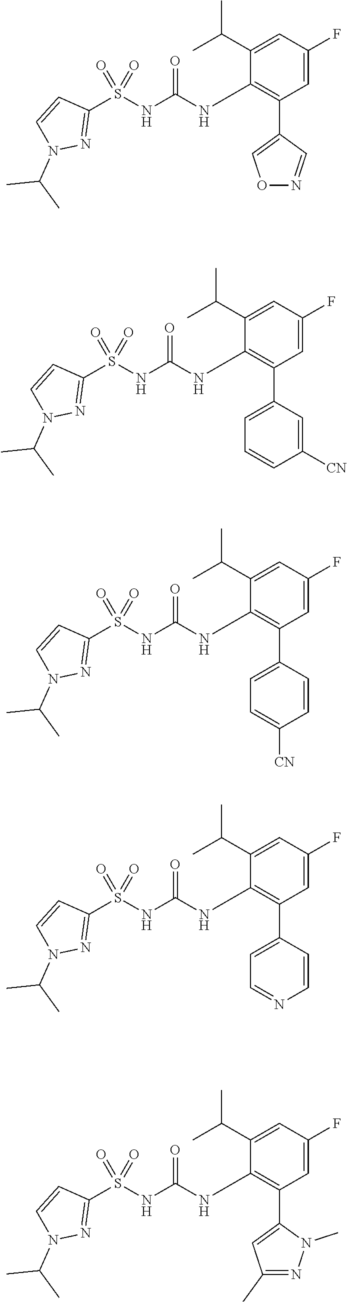 [SMILES: CC(C)c1cc(F)cc(-c2cnoc2)c1NC(=O)NS(=O)(=O)c1ccn(C(C)C)n1 ; CC(C)c1cc(F)cc(-c2cccc(C#N)c2)c1NC(=O)NS(=O)(=O)c1ccn(C(C)C)n1 ; CC(C)c1cc(F)cc(-c2ccc(C#N)cc2)c1NC(=O)NS(=O)(=O)c1ccn(C(C)C)n1 ; CC(C)c1cc(F)cc(-c2ccncc2)c1NC(=O)NS(=O)(=O)c1ccn(C(C)C)n1 ; Cc1cc(-c2cc(F)cc(C(C)C)c2NC(=O)NS(=O)(=O)c2ccn(C(C)C)n2)n(C)n1]

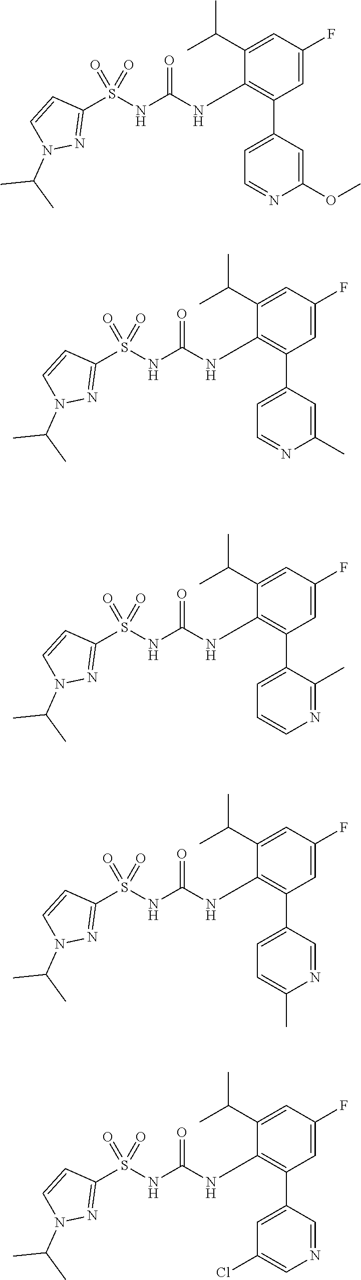

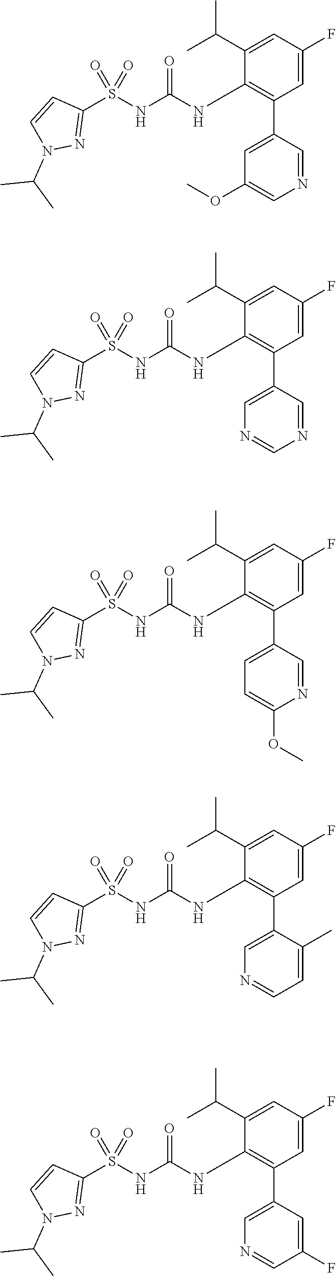

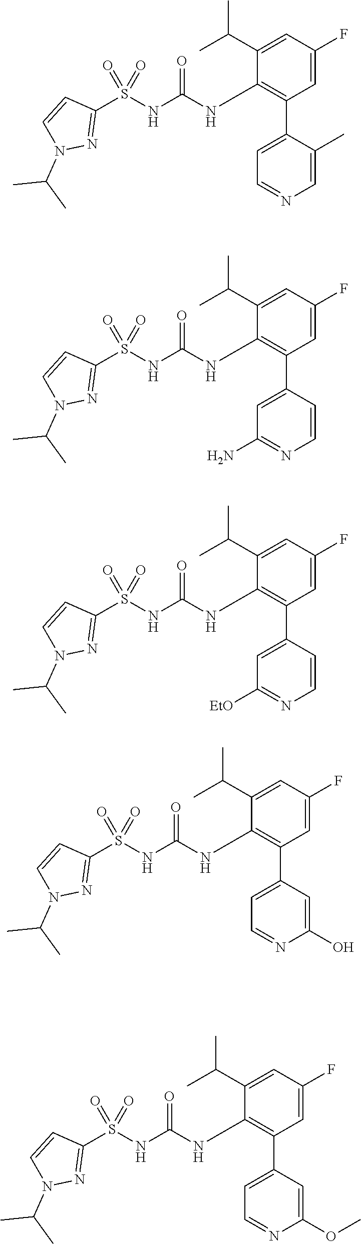

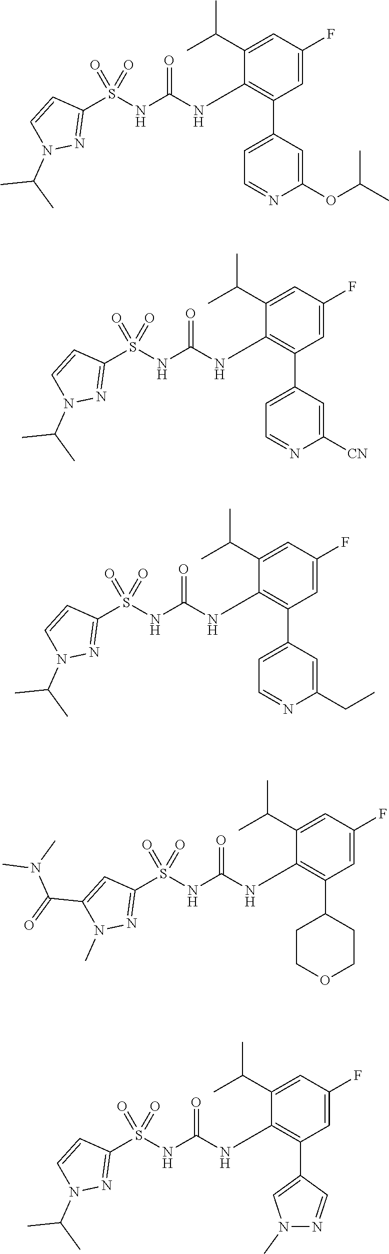

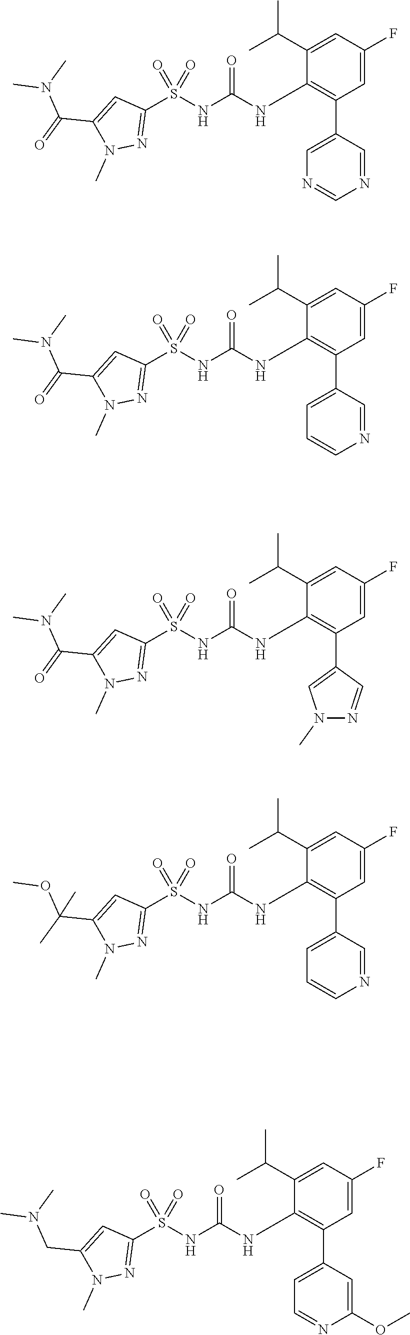

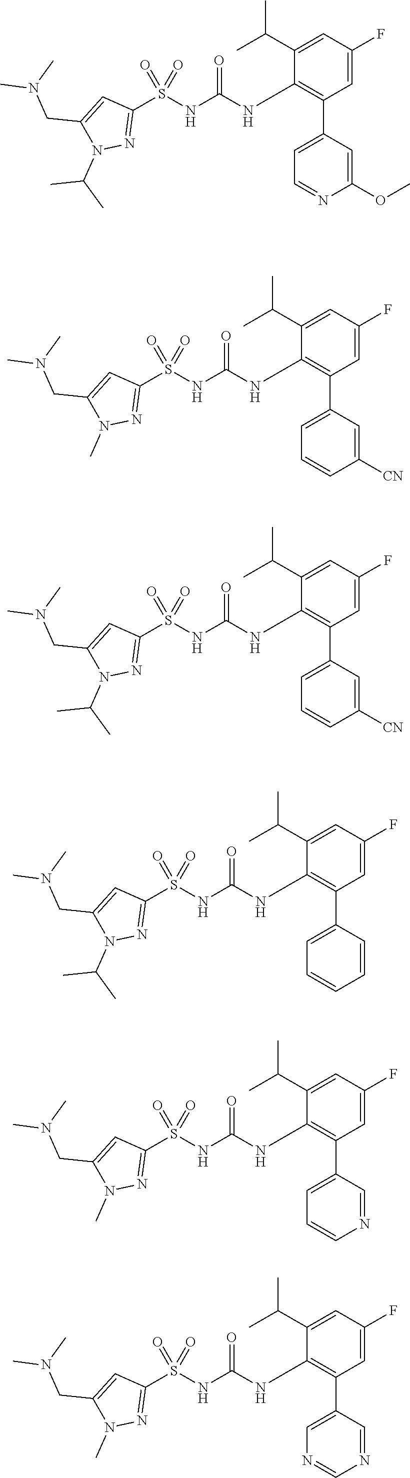

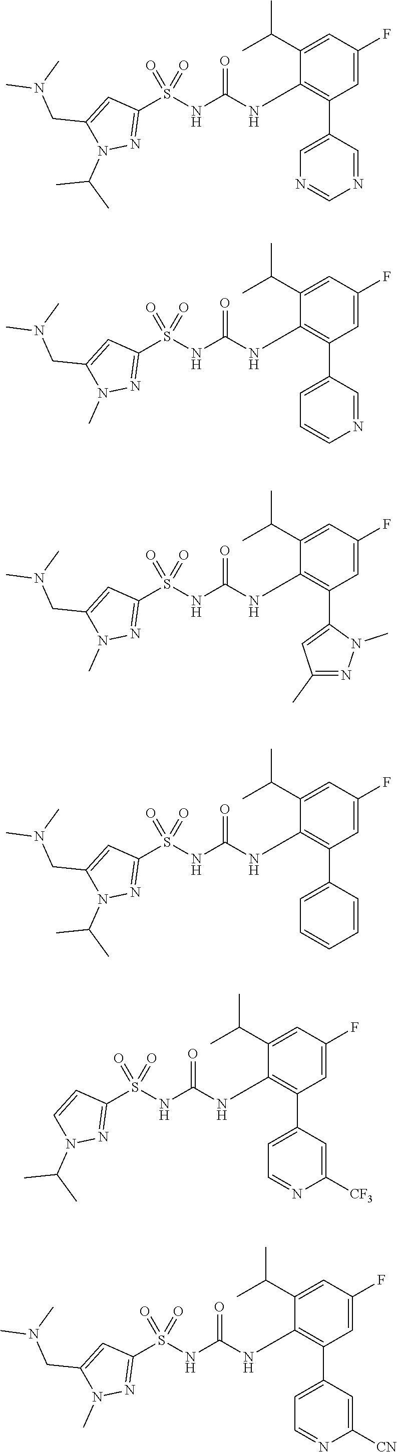

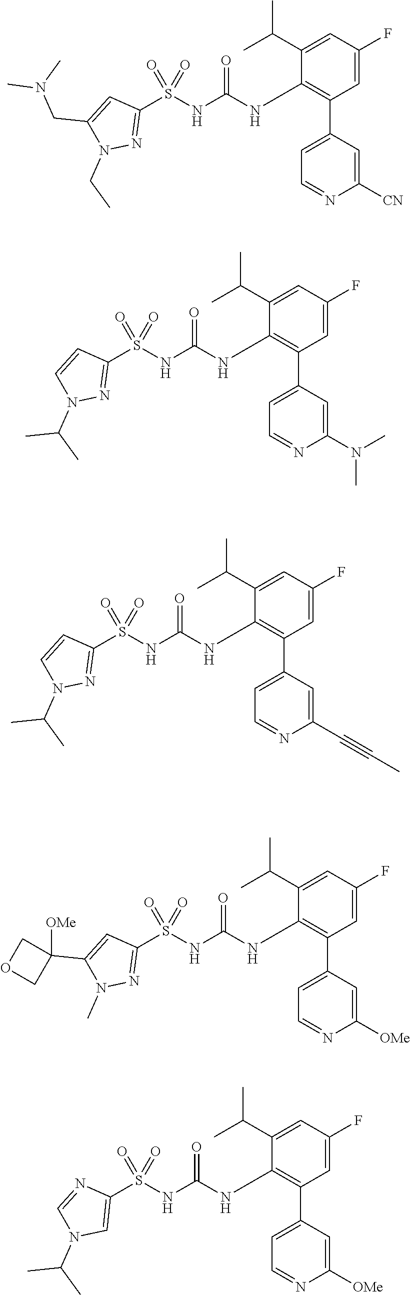

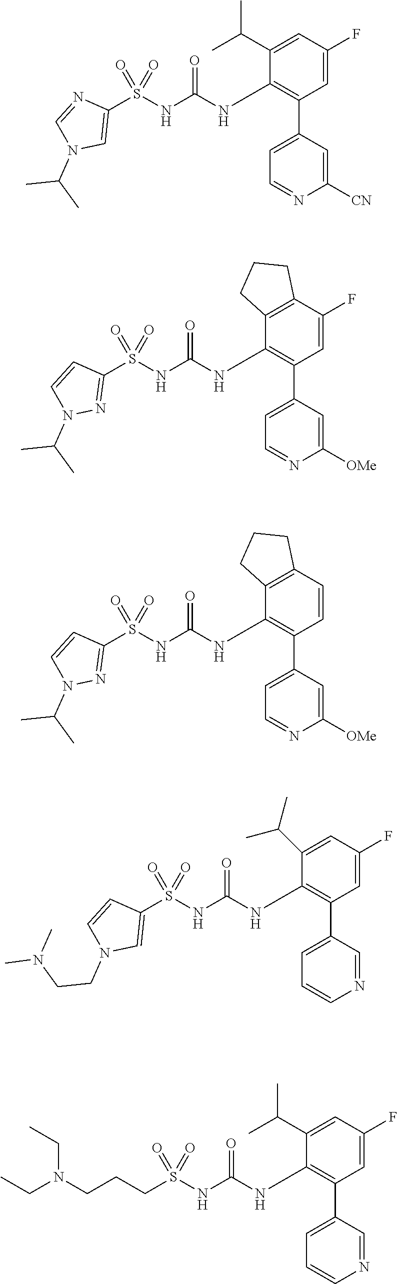 [SMILES: CC(C)c1cc(F)cc(-c2ccnc(C#N)c2)c1NC(=O)NS(=O)(=O)c1cn(C(C)C)cn1 ; COc1cc(-c2cc(F)c3c(c2NC(=O)NS(=O)(=O)c2ccn(C(C)C)n2)CCC3)ccn1 ; COc1cc(-c2ccc3c(c2NC(=O)NS(=O)(=O)c2ccn(C(C)C)n2)CCC3)ccn1 ; CC(C)c1cc(F)cc(-c2cccnc2)c1NC(=O)NS(=O)(=O)c1ccn(CCN(C)C)c1 ; CCN(CC)CCCS(=O)(=O)NC(=O)Nc1c(-c2cccnc2)cc(F)cc1C(C)C]

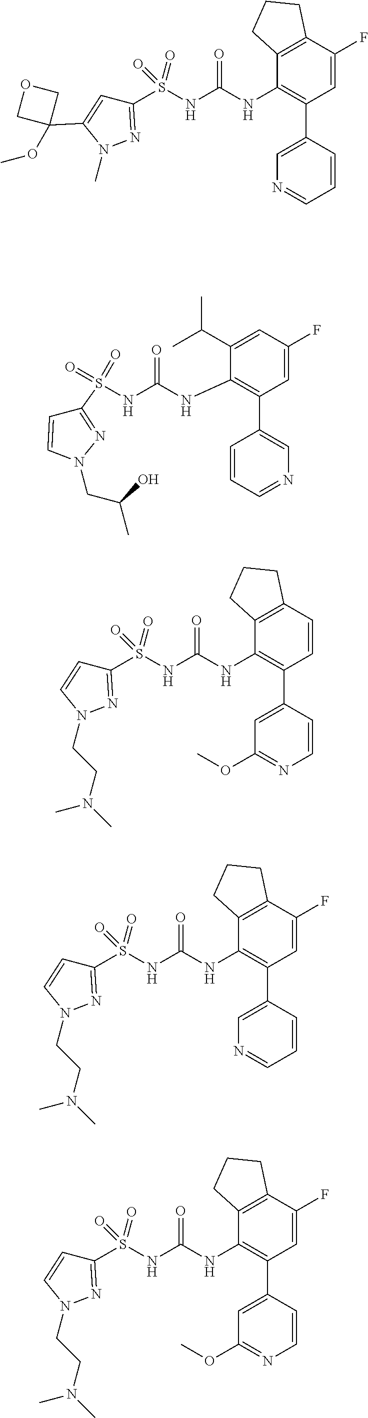 [SMILES: COC1(c2cc(S(=O)(=O)NC(=O)Nc3c(-c4cccnc4)cc(F)c4c3CCC4)nn2C)COC1 ; CC(C)c1cc(F)cc(-c2cccnc2)c1NC(=O)NS(=O)(=O)c1ccn(C[C@H](C)O)n1 ; COc1cc(-c2ccc3c(c2NC(=O)NS(=O)(=O)c2ccn(CCN(C)C)n2)CCC3)ccn1 ; CN(C)CCn1ccc(S(=O)(=O)NC(=O)Nc2c(-c3cccnc3)cc(F)c3c2CCC3)n1 ; COc1cc(-c2cc(F)c3c(c2NC(=O)NS(=O)(=O)c2ccn(CCN(C)C)n2)CCC3)ccn1]

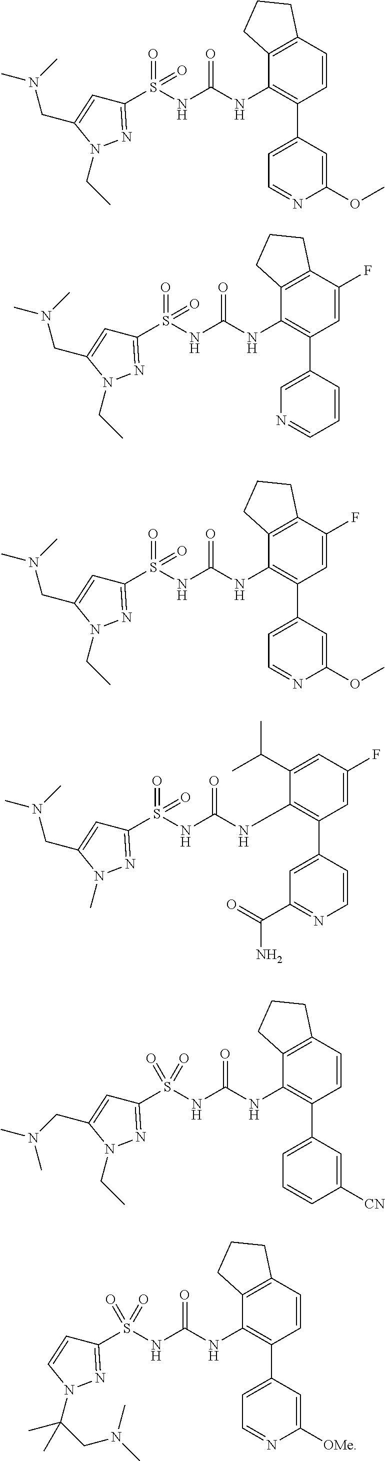

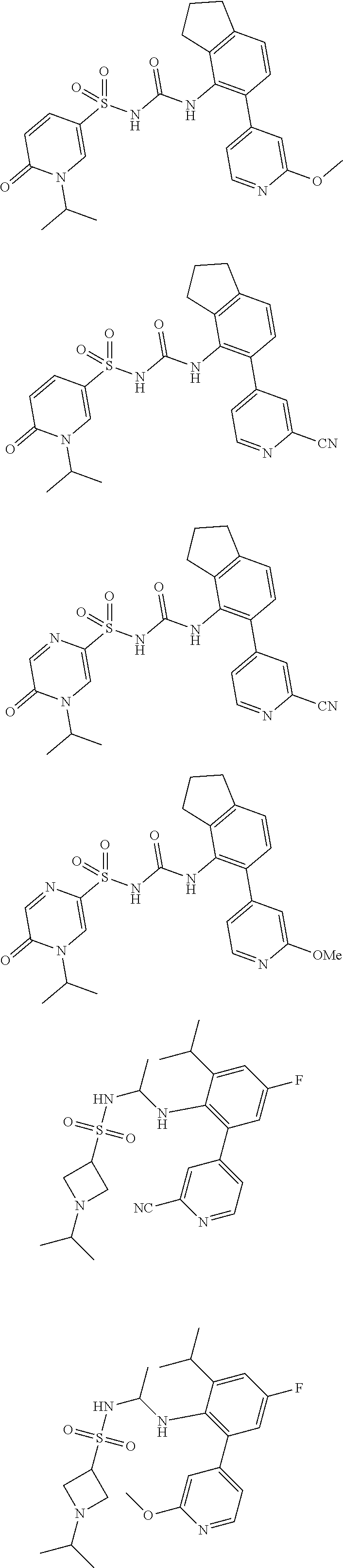

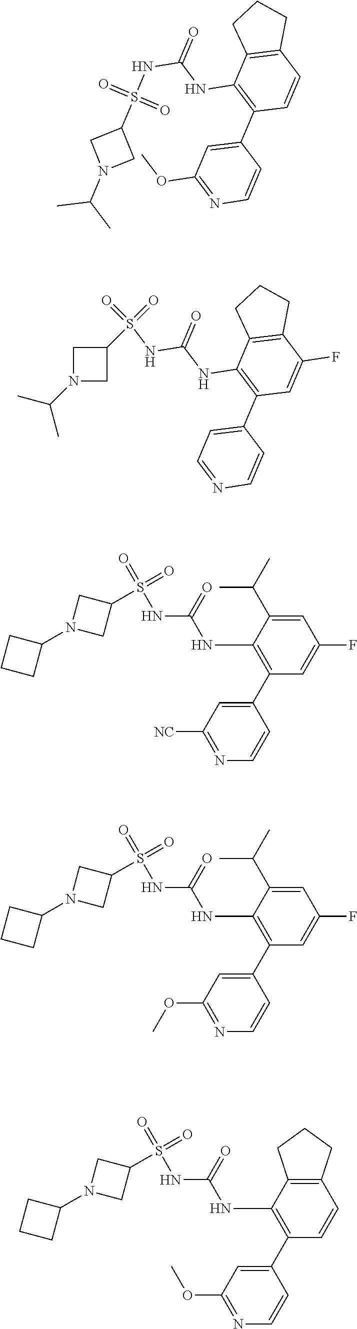 [SMILES: COc1cc(-c2ccc3c(c2NC(=O)NS(=O)(=O)C2CN(C(C)C)C2)CCC3)ccn1 ; CC(C)N1CC(S(=O)(=O)NC(=O)Nc2c(-c3ccncc3)cc(F)c3c2CCC3)C1 ; CC(C)c1cc(F)cc(-c2ccnc(C#N)c2)c1NC(=O)NS(=O)(=O)C1CN(C2CCC2)C1 ; COc1cc(-c2cc(F)cc(C(C)C)c2NC(=O)NS(=O)(=O)C2CN(C3CCC3)C2)ccn1 ; COc1cc(-c2ccc3c(c2NC(=O)NS(=O)(=O)C2CN(C4CCC4)C2)CCC3)ccn1]

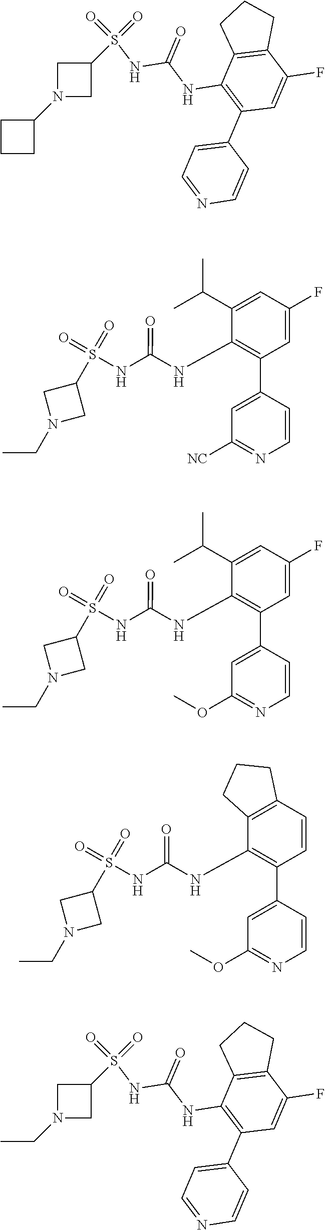 [SMILES: O=C(Nc1c(-c2ccncc2)cc(F)c2c1CCC2)NS(=O)(=O)C1CN(C2CCC2)C1 ; CCN1CC(S(=O)(=O)NC(=O)Nc2c(-c3ccnc(C#N)c3)cc(F)cc2C(C)C)C1 ; CCN1CC(S(=O)(=O)NC(=O)Nc2c(-c3ccnc(OC)c3)cc(F)cc2C(C)C)C1 ; CCN1CC(S(=O)(=O)NC(=O)Nc2c(-c3ccnc(OC)c3)ccc3c2CCC3)C1 ; CCN1CC(S(=O)(=O)NC(=O)Nc2c(-c3ccncc3)cc(F)c3c2CCC3)C1]

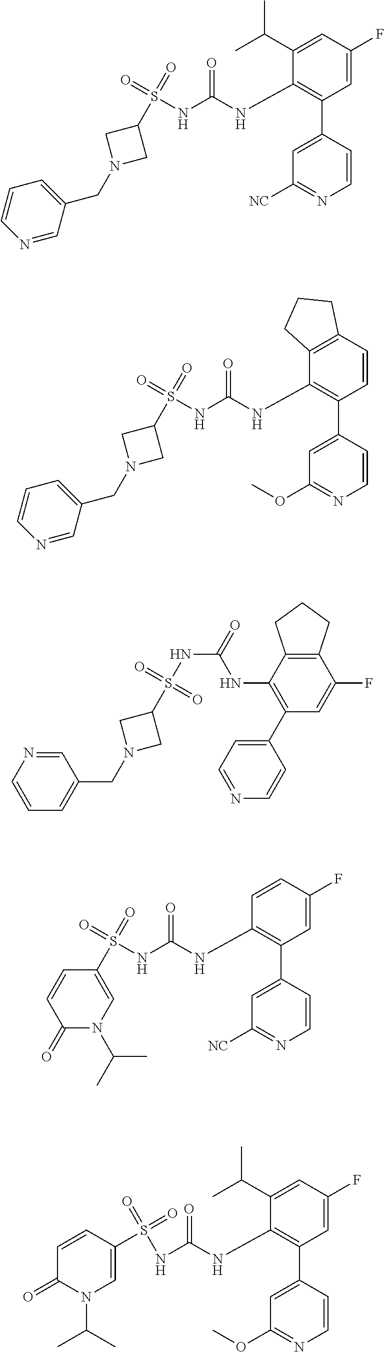 [SMILES: CC(C)c1cc(F)cc(-c2ccnc(C#N)c2)c1NC(=O)NS(=O)(=O)C1CN(Cc2cccnc2)C1 ; COc1cc(-c2ccc3c(c2NC(=O)NS(=O)(=O)C2CN(Cc4cccnc4)C2)CCC3)ccn1 ; O=C(Nc1c(-c2ccncc2)cc(F)c2c1CCC2)NS(=O)(=O)C1CN(Cc2cccnc2)C1 ; CC(C)n1cc(S(=O)(=O)NC(=O)Nc2ccc(F)cc2-c2ccnc(C#N)c2)ccc1=O ; COc1cc(-c2cc(F)cc(C(C)C)c2NC(=O)NS(=O)(=O)c2ccc(=O)n(C(C)C)c2)ccn1]

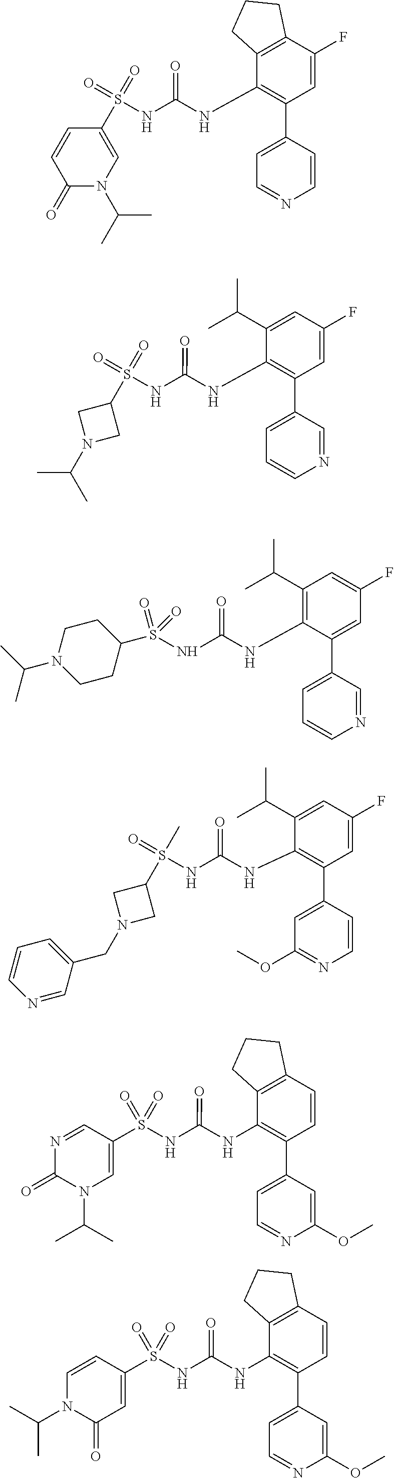

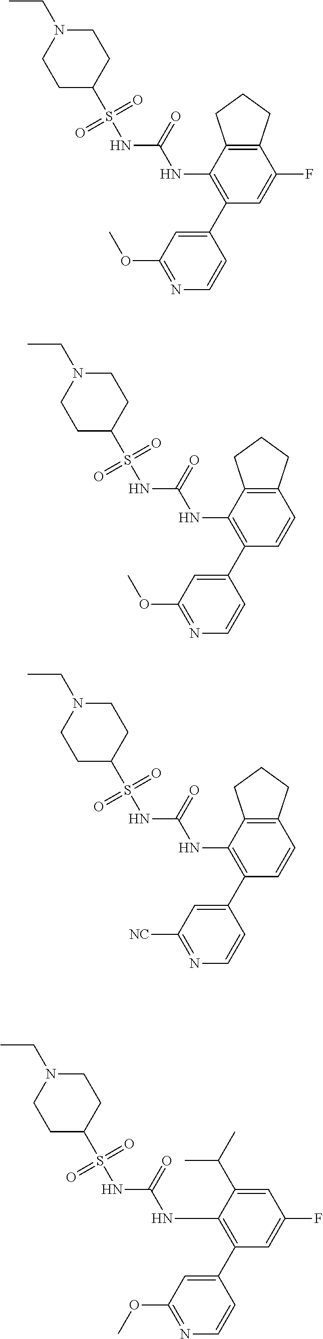 [SMILES: CCN1CCC(S(=O)(=O)NC(=O)Nc2c(-c3ccnc(OC)c3)cc(F)c3c2CCC3)CC1 ; CCN1CCC(S(=O)(=O)NC(=O)Nc2c(-c3ccnc(OC)c3)ccc3c2CCC3)CC1 ; CCN1CCC(S(=O)(=O)NC(=O)Nc2c(-c3ccnc(C#N)c3)ccc3c2CCC3)CC1 ; CCN1CCC(S(=O)(=O)NC(=O)Nc2c(-c3ccnc(OC)c3)cc(F)cc2C(C)C)CC1]

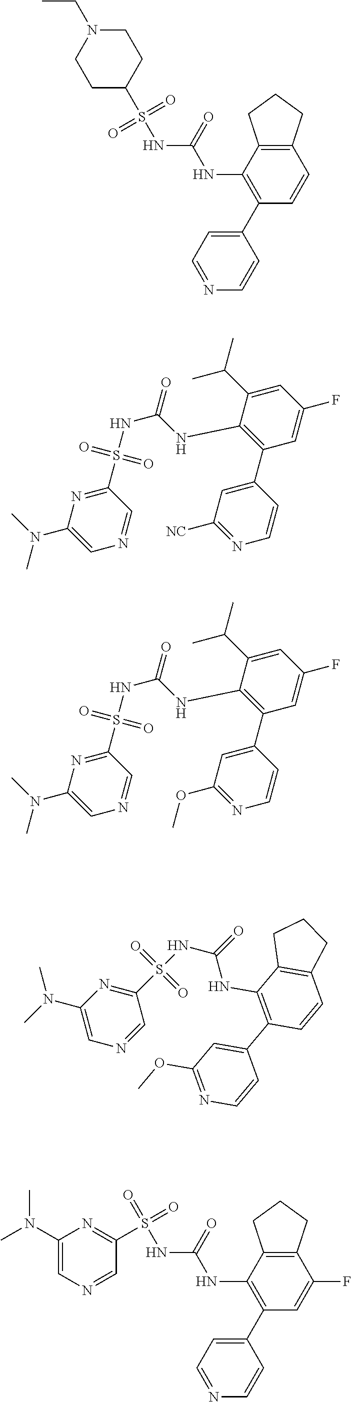 [SMILES: CCN1CCC(S(=O)(=O)NC(=O)Nc2c(-c3ccncc3)ccc3c2CCC3)CC1 ; CC(C)c1cc(F)cc(-c2ccnc(C#N)c2)c1NC(=O)NS(=O)(=O)c1cncc(N(C)C)n1 ; COc1cc(-c2cc(F)cc(C(C)C)c2NC(=O)NS(=O)(=O)c2cncc(N(C)C)n2)ccn1 ; COc1cc(-c2ccc3c(c2NC(=O)NS(=O)(=O)c2cncc(N(C)C)n2)CCC3)ccn1 ; CN(C)c1cncc(S(=O)(=O)NC(=O)Nc2c(-c3ccncc3)cc(F)c3c2CCC3)n1]

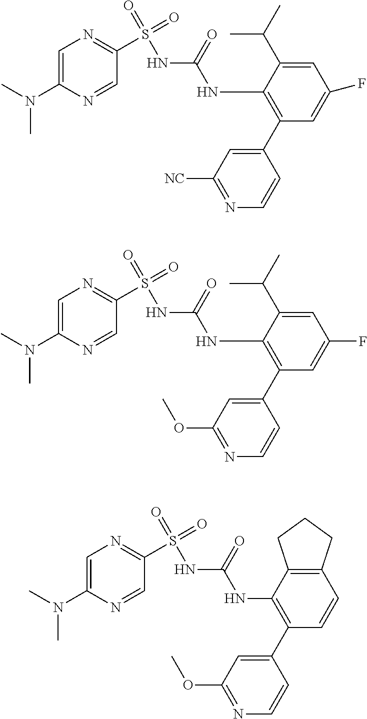 [SMILES: CC(C)c1cc(F)cc(-c2ccnc(C#N)c2)c1NC(=O)NS(=O)(=O)c1cnc(N(C)C)cn1 ; COc1cc(-c2cc(F)cc(C(C)C)c2NC(=O)NS(=O)(=O)c2cnc(N(C)C)cn2)ccn1 ; COc1cc(-c2ccc3c(c2NC(=O)NS(=O)(=O)c2cnc(N(C)C)cn2)CCC3)ccn1]

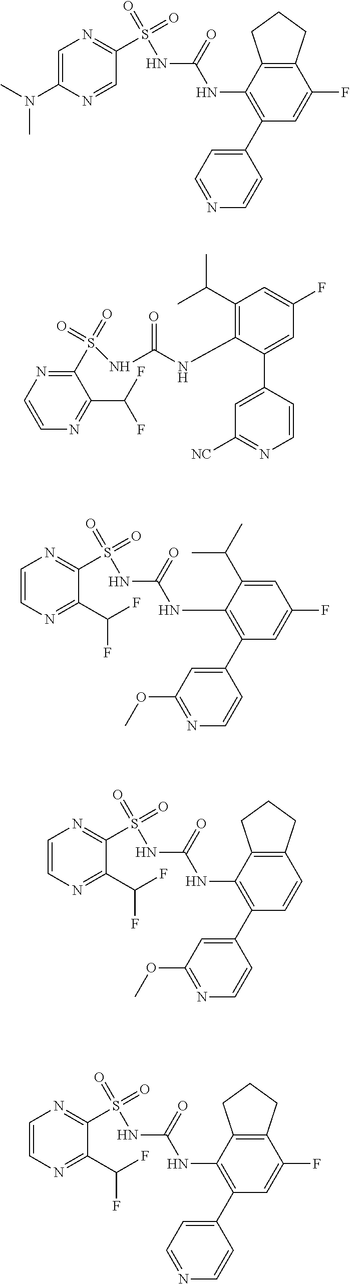

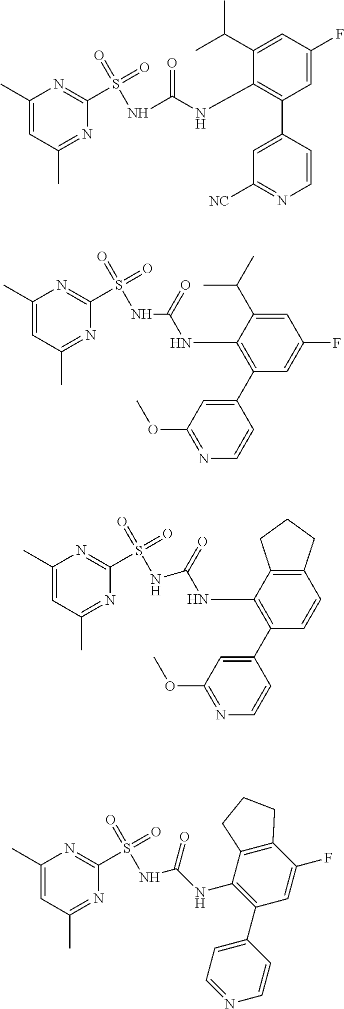 [SMILES: Cc1cc(C)nc(S(=O)(=O)NC(=O)Nc2c(-c3ccnc(C#N)c3)cc(F)cc2C(C)C)n1 ; COc1cc(-c2cc(F)cc(C(C)C)c2NC(=O)NS(=O)(=O)c2nc(C)cc(C)n2)ccn1 ; COc1cc(-c2ccc3c(c2NC(=O)NS(=O)(=O)c2nc(C)cc(C)n2)CCC3)ccn1 ; Cc1cc(C)nc(S(=O)(=O)NC(=O)Nc2c(-c3ccncc3)cc(F)c3c2CCC3)n1]

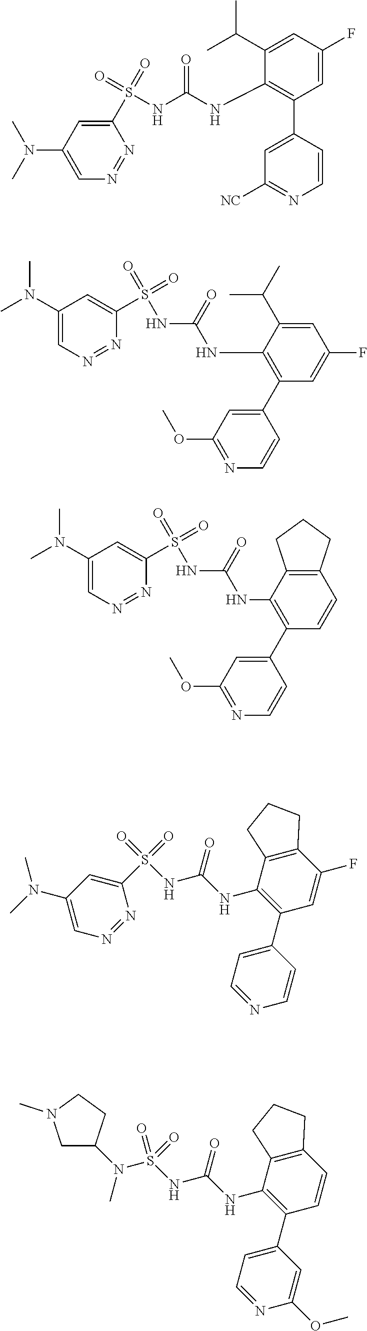

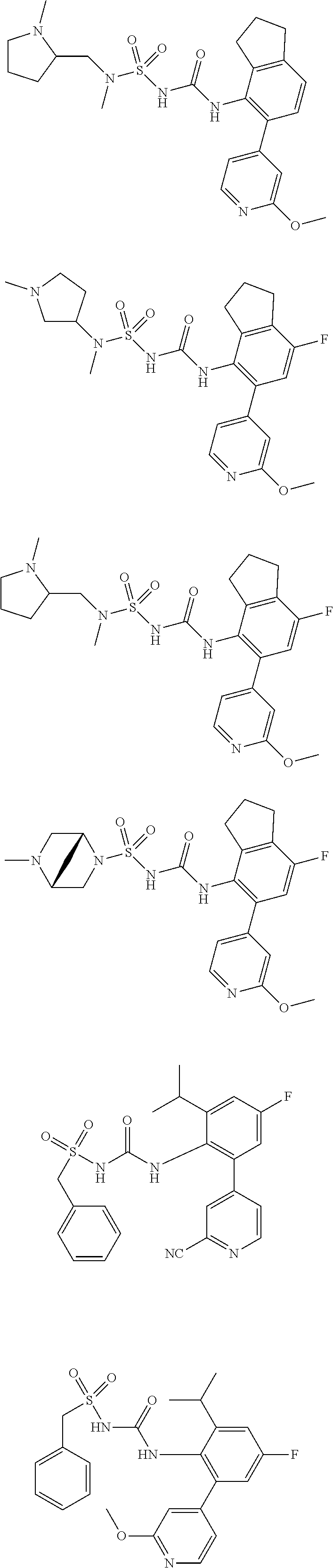

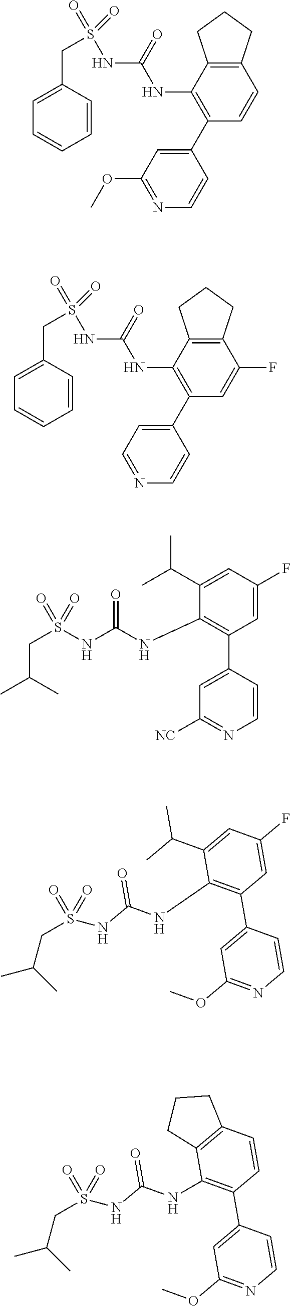 [SMILES: COc1cc(-c2ccc3c(c2NC(=O)NS(=O)(=O)Cc2ccccc2)CCC3)ccn1 ; O=C(Nc1c(-c2ccncc2)cc(F)c2c1CCC2)NS(=O)(=O)Cc1ccccc1 ; CC(C)CS(=O)(=O)NC(=O)Nc1c(-c2ccnc(C#N)c2)cc(F)cc1C(C)C ; COc1cc(-c2cc(F)cc(C(C)C)c2NC(=O)NS(=O)(=O)CC(C)C)ccn1 ; COc1cc(-c2ccc3c(c2NC(=O)NS(=O)(=O)CC(C)C)CCC3)ccn1]

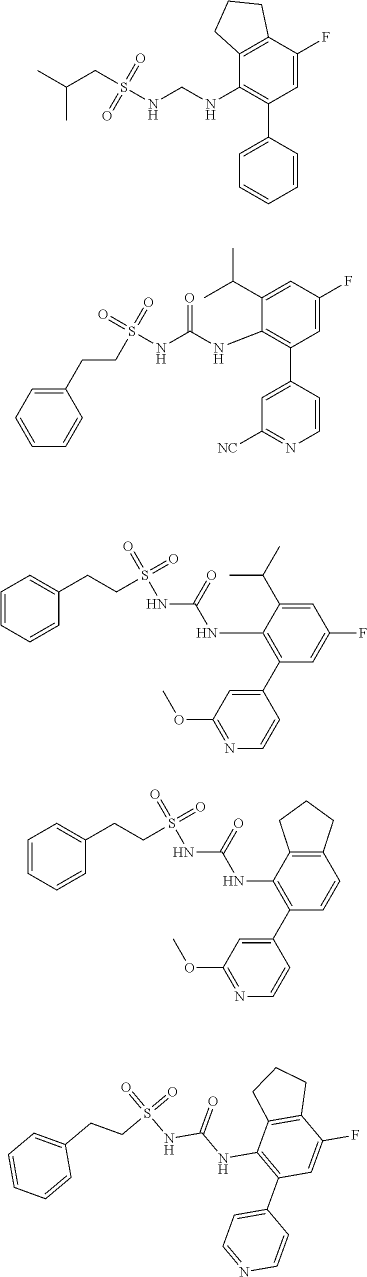 [SMILES: CC(C)CS(=O)(=O)NCNc1c(-c2ccccc2)cc(F)c2c1CCC2 ; CC(C)c1cc(F)cc(-c2ccnc(C#N)c2)c1NC(=O)NS(=O)(=O)CCc1ccccc1 ; COc1cc(-c2cc(F)cc(C(C)C)c2NC(=O)NS(=O)(=O)CCc2ccccc2)ccn1 ; COc1cc(-c2ccc3c(c2NC(=O)NS(=O)(=O)CCc2ccccc2)CCC3)ccn1 ; O=C(Nc1c(-c2ccncc2)cc(F)c2c1CCC2)NS(=O)(=O)CCc1ccccc1]

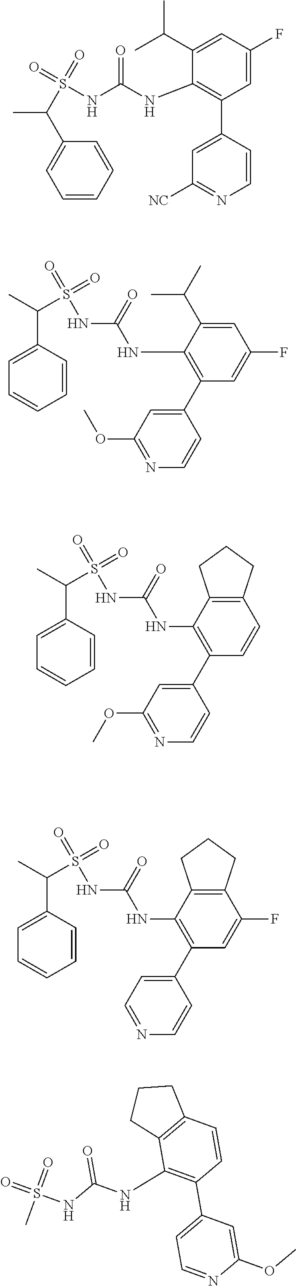 [SMILES: CC(C)c1cc(F)cc(-c2ccnc(C#N)c2)c1NC(=O)NS(=O)(=O)C(C)c1ccccc1 ; COc1cc(-c2cc(F)cc(C(C)C)c2NC(=O)NS(=O)(=O)C(C)c2ccccc2)ccn1 ; COc1cc(-c2ccc3c(c2NC(=O)NS(=O)(=O)C(C)c2ccccc2)CCC3)ccn1 ; CC(c1ccccc1)S(=O)(=O)NC(=O)Nc1c(-c2ccncc2)cc(F)c2c1CCC2 ; COc1cc(-c2ccc3c(c2NC(=O)NS(C)(=O)=O)CCC3)ccn1]

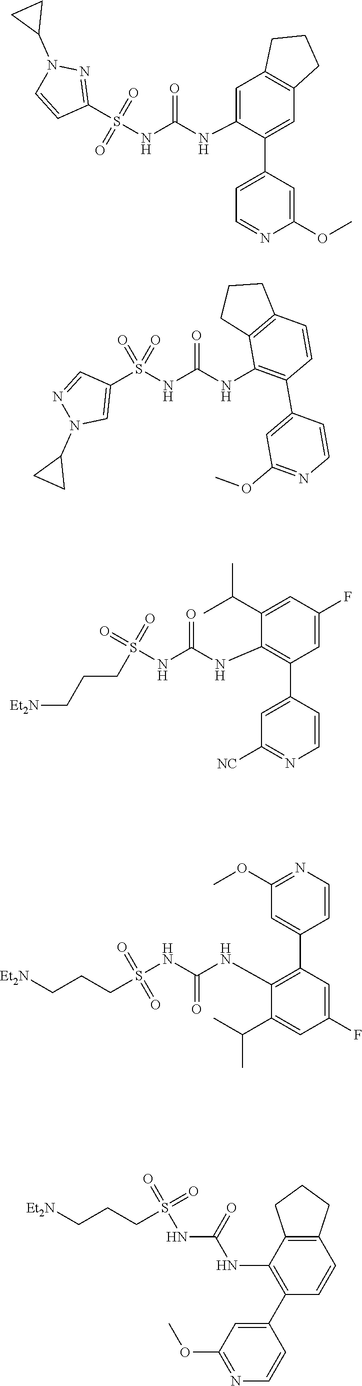

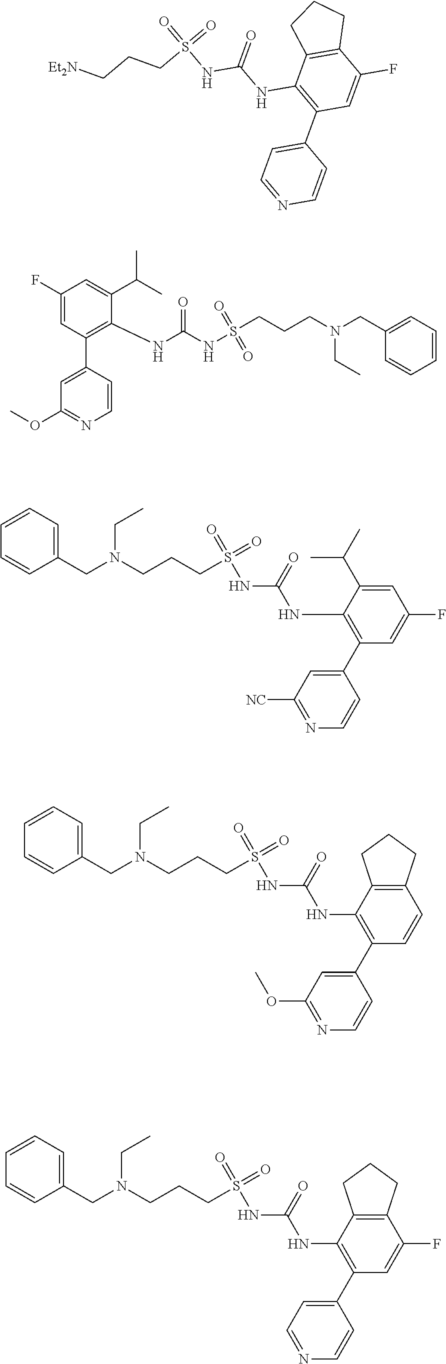

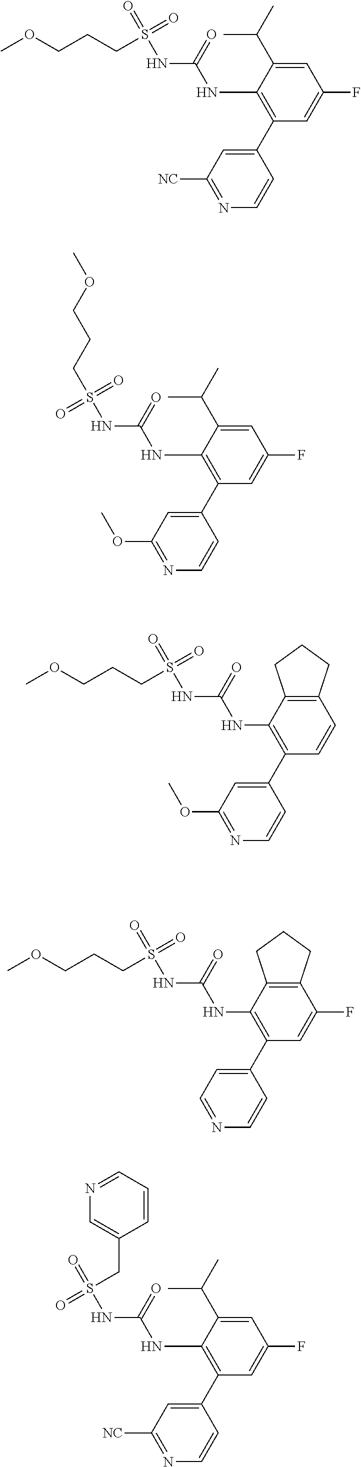 [SMILES: COCCCS(=O)(=O)NC(=O)Nc1c(-c2ccnc(C#N)c2)cc(F)cc1C(C)C ; COCCCS(=O)(=O)NC(=O)Nc1c(-c2ccnc(OC)c2)cc(F)cc1C(C)C ; COCCCS(=O)(=O)NC(=O)Nc1c(-c2ccnc(OC)c2)ccc2c1CCC2 ; COCCCS(=O)(=O)NC(=O)Nc1c(-c2ccncc2)cc(F)c2c1CCC2 ; CC(C)c1cc(F)cc(-c2ccnc(C#N)c2)c1NC(=O)NS(=O)(=O)Cc1cccnc1]

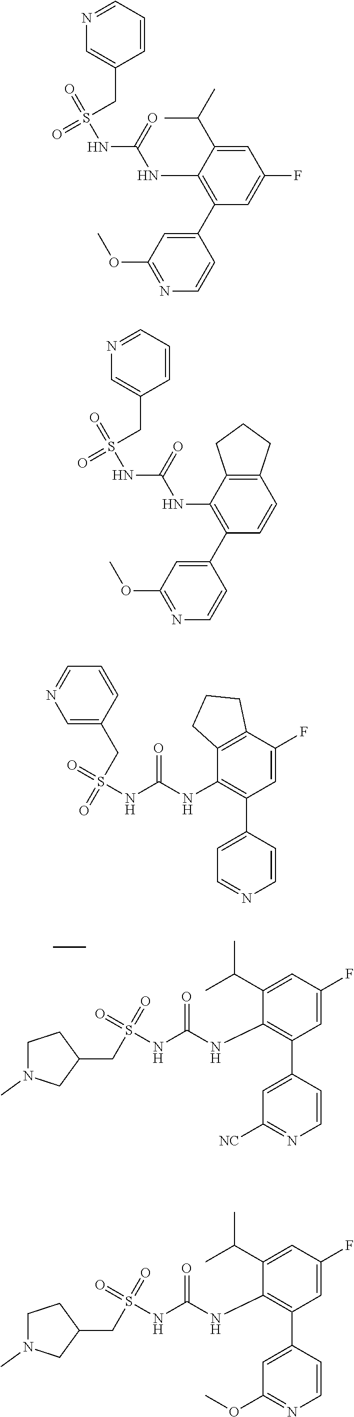

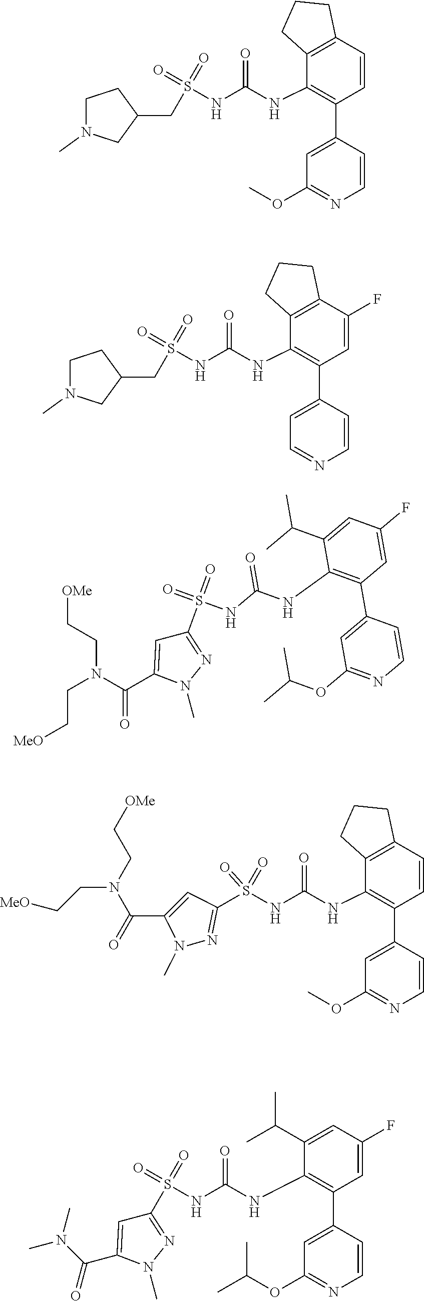

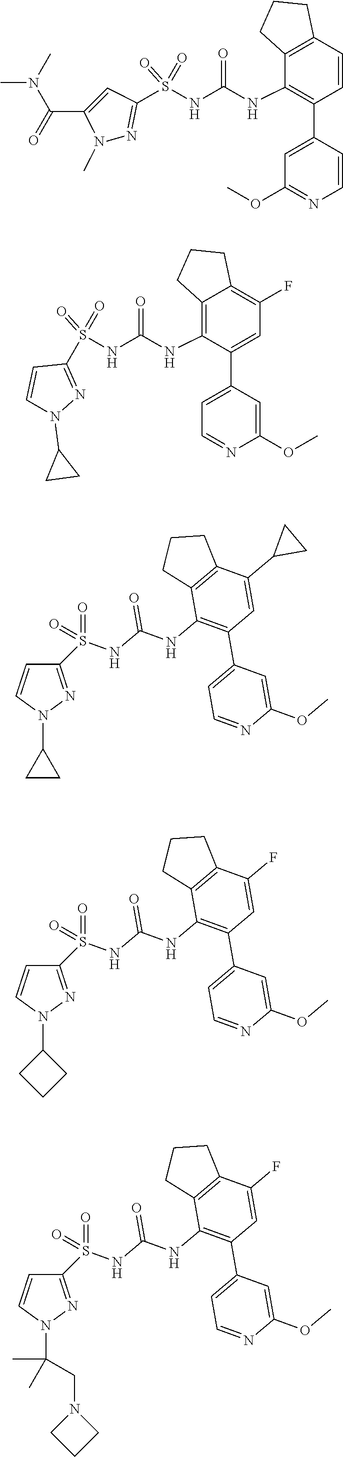

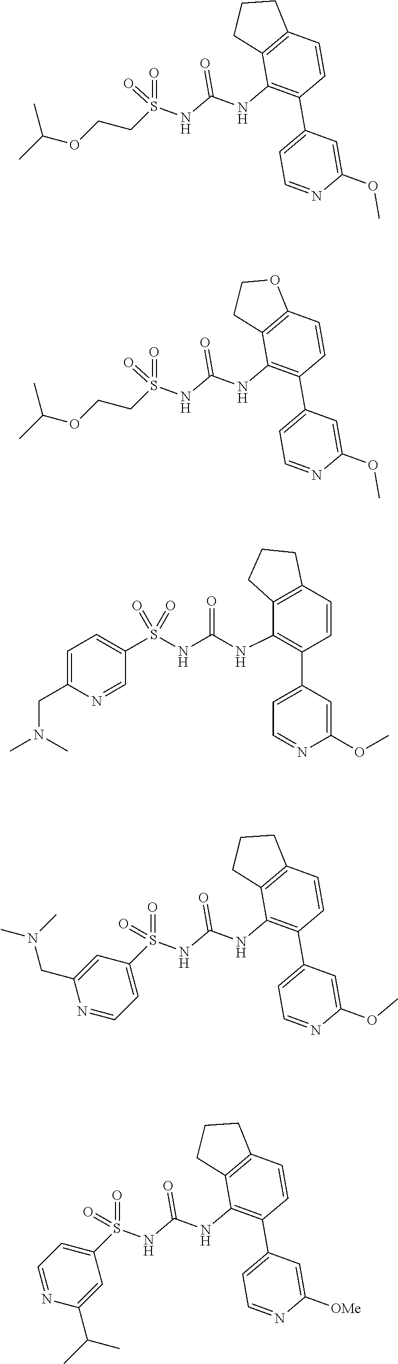

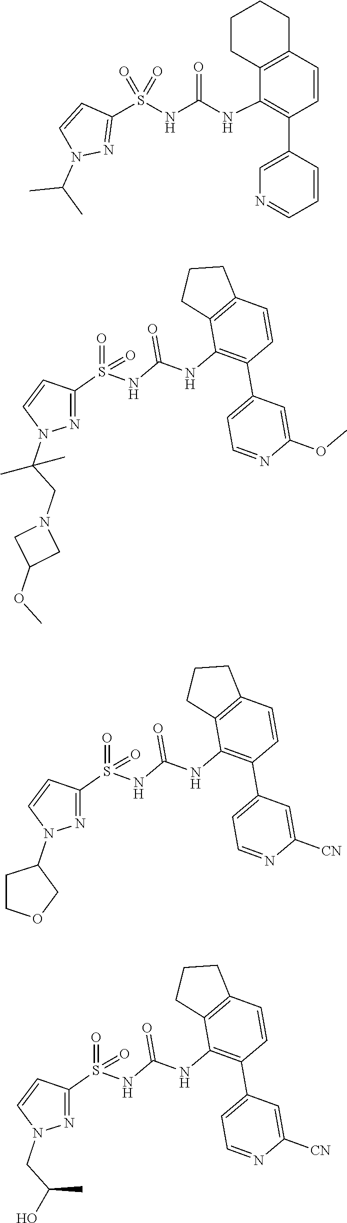 [SMILES: CC(C)n1ccc(S(=O)(=O)NC(=O)Nc2c(-c3cccnc3)ccc3c2CCCC3)n1 ; COc1cc(-c2ccc3c(c2NC(=O)NS(=O)(=O)c2ccn(C(C)(C)CN4CC(OC)C4)n2)CCC3)ccn1 ; N#Cc1cc(-c2ccc3c(c2NC(=O)NS(=O)(=O)c2ccn(C4CCOC4)n2)CCC3)ccn1 ; C[C@@H](O)Cn1ccc(S(=O)(=O)NC(=O)Nc2c(-c3ccnc(C#N)c3)ccc3c2CCC3)n1]

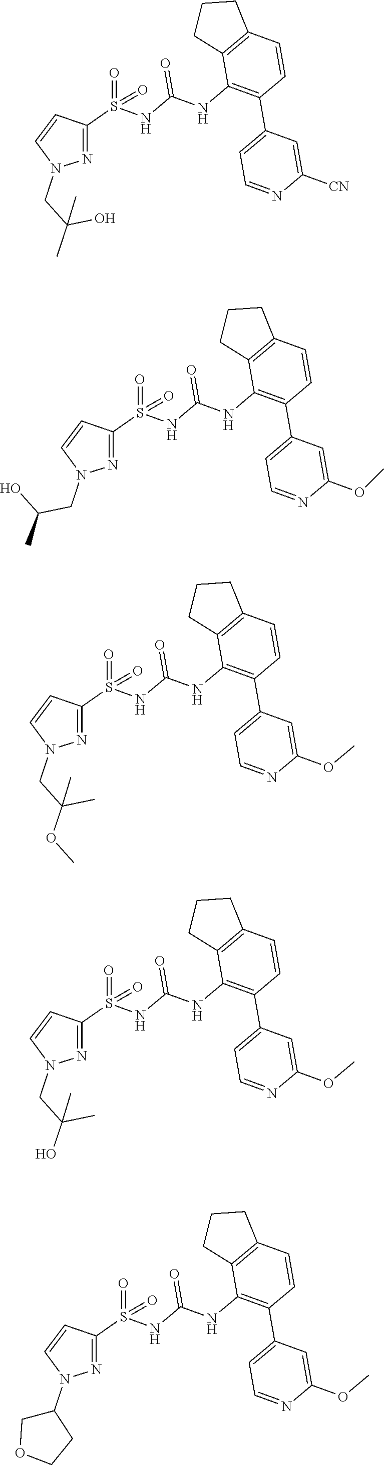 [SMILES: CC(C)(O)Cn1ccc(S(=O)(=O)NC(=O)Nc2c(-c3ccnc(C#N)c3)ccc3c2CCC3)n1 ; COc1cc(-c2ccc3c(c2NC(=O)NS(=O)(=O)c2ccn(C[C@@H](C)O)n2)CCC3)ccn1 ; COc1cc(-c2ccc3c(c2NC(=O)NS(=O)(=O)c2ccn(CC(C)(C)OC)n2)CCC3)ccn1 ; COc1cc(-c2ccc3c(c2NC(=O)NS(=O)(=O)c2ccn(CC(C)(C)O)n2)CCC3)ccn1 ; COc1cc(-c2ccc3c(c2NC(=O)NS(=O)(=O)c2ccn(C4CCOC4)n2)CCC3)ccn1]

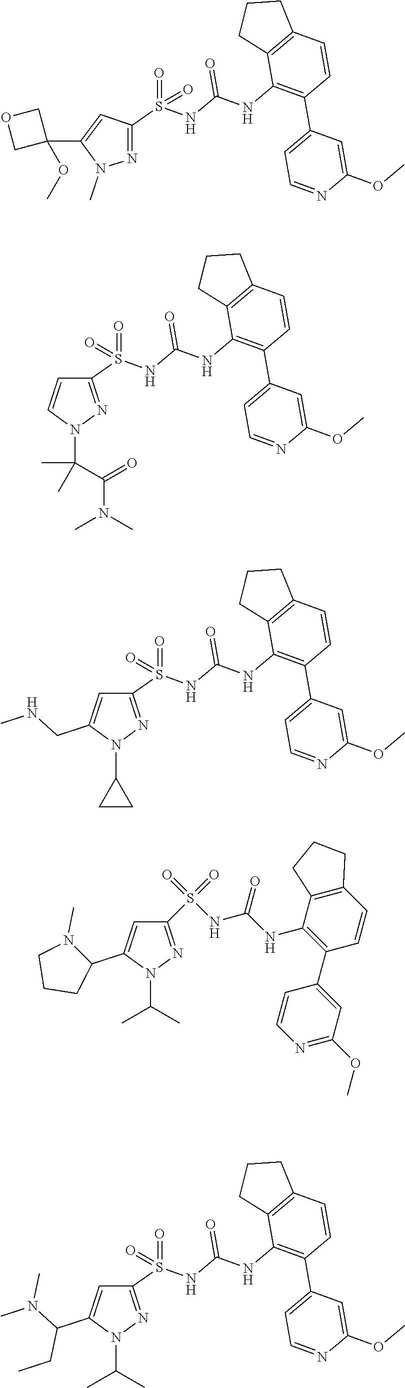

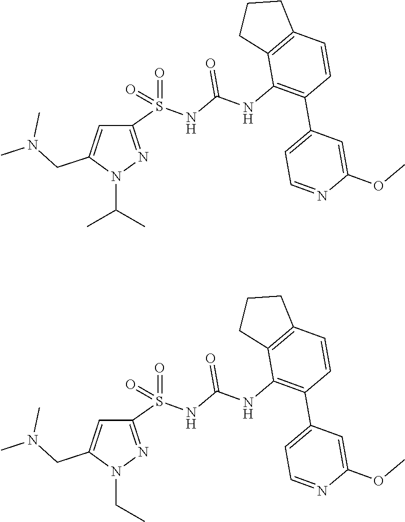

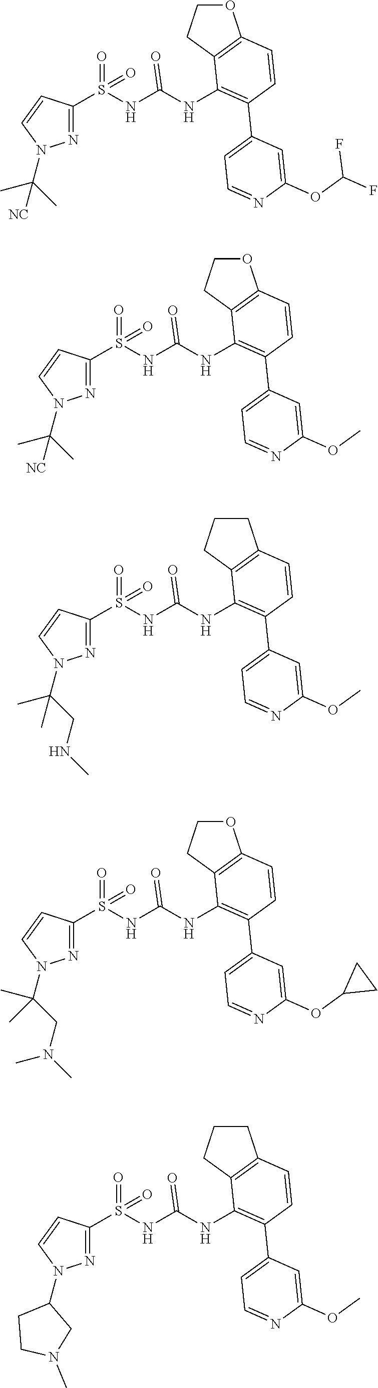

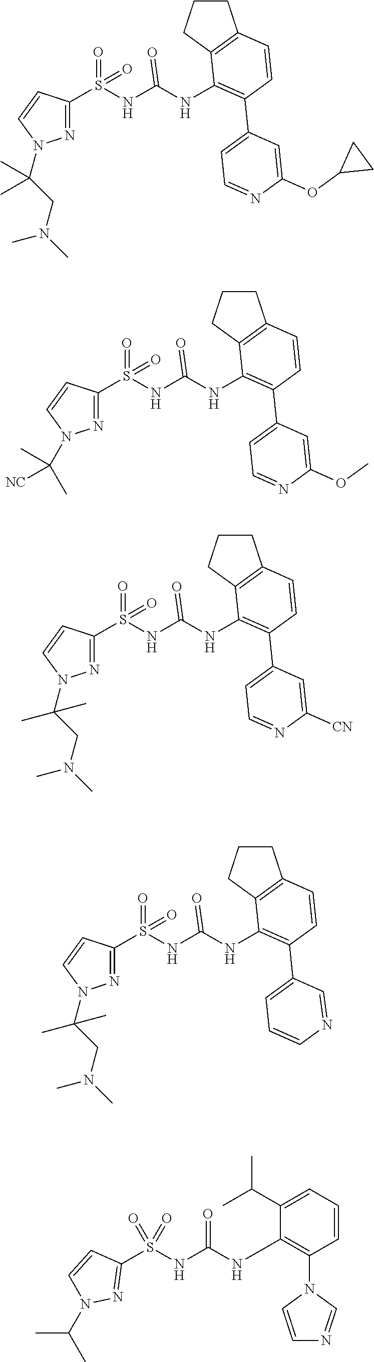

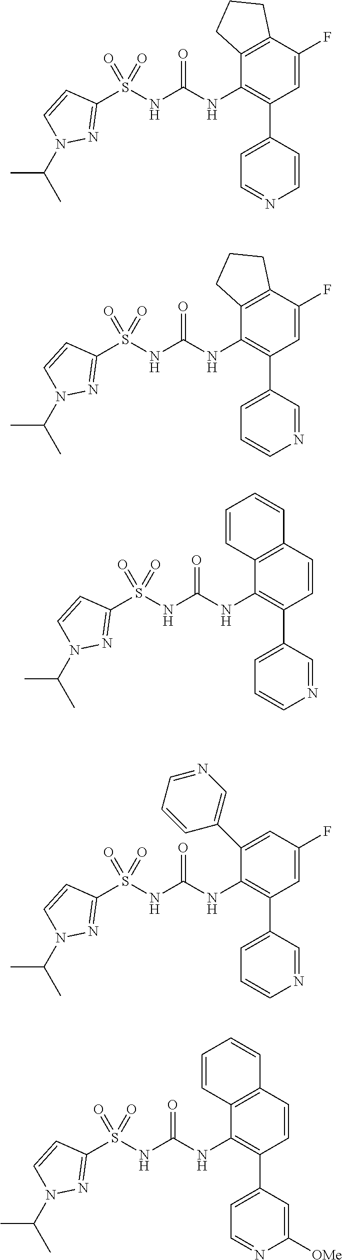

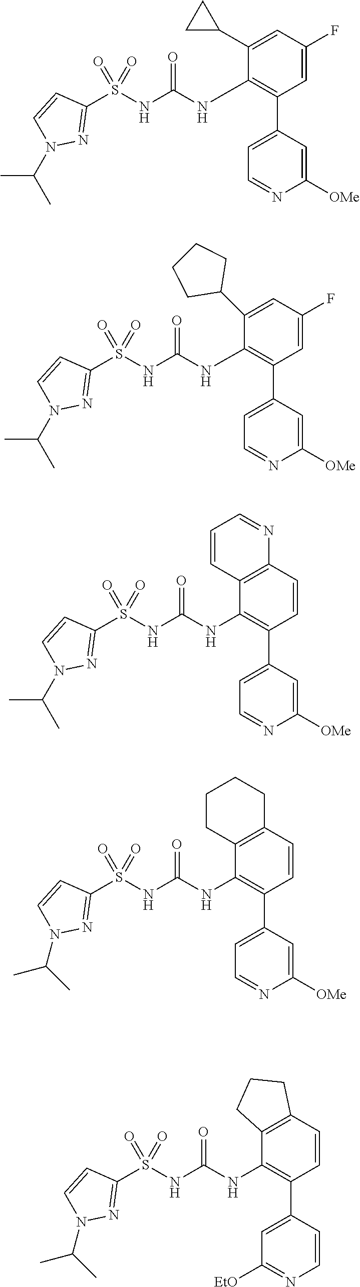 [SMILES: COc1cc(-c2cc(F)cc(C3CC3)c2NC(=O)NS(=O)(=O)c2ccn(C(C)C)n2)ccn1 ; COc1cc(-c2cc(F)cc(C3CCCC3)c2NC(=O)NS(=O)(=O)c2ccn(C(C)C)n2)ccn1 ; COc1cc(-c2ccc3ncccc3c2NC(=O)NS(=O)(=O)c2ccn(C(C)C)n2)ccn1 ; COc1cc(-c2ccc3c(c2NC(=O)NS(=O)(=O)c2ccn(C(C)C)n2)CCCC3)ccn1 ; CCOc1cc(-c2ccc3c(c2NC(=O)NS(=O)(=O)c2ccn(C(C)C)n2)CCC3)ccn1]

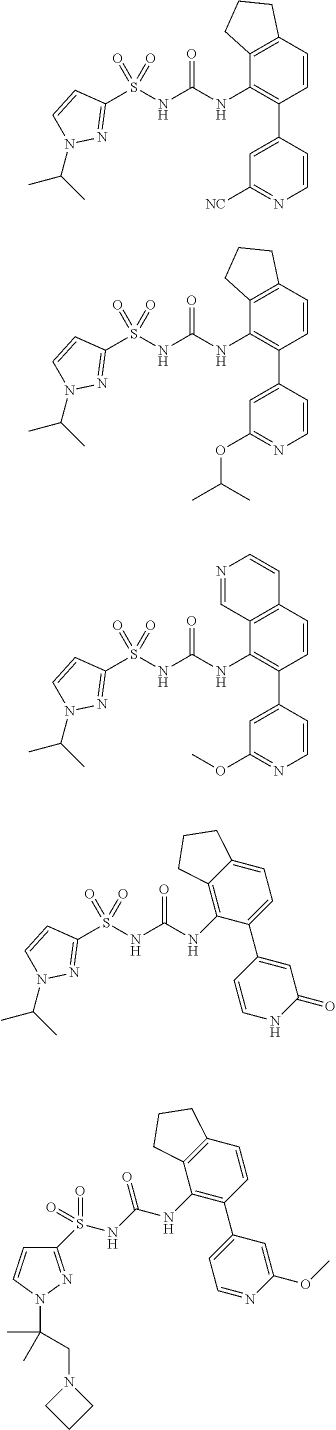 [SMILES: CC(C)n1ccc(S(=O)(=O)NC(=O)Nc2c(-c3ccnc(C#N)c3)ccc3c2CCC3)n1 ; CC(C)Oc1cc(-c2ccc3c(c2NC(=O)NS(=O)(=O)c2ccn(C(C)C)n2)CCC3)ccn1 ; COc1cc(-c2ccc3ccncc3c2NC(=O)NS(=O)(=O)c2ccn(C(C)C)n2)ccn1 ; CC(C)n1ccc(S(=O)(=O)NC(=O)Nc2c(-c3cc[nH]c(=O)c3)ccc3c2CCC3)n1 ; COc1cc(-c2ccc3c(c2NC(=O)NS(=O)(=O)c2ccn(C(C)(C)CN4CCC4)n2)CCC3)ccn1]

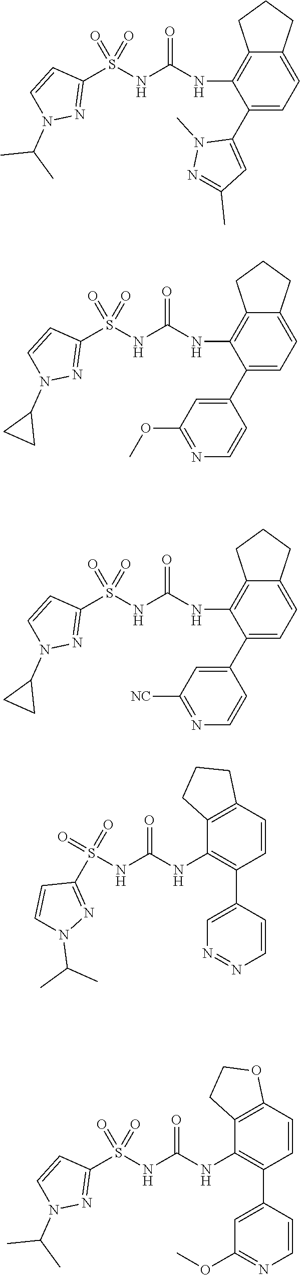

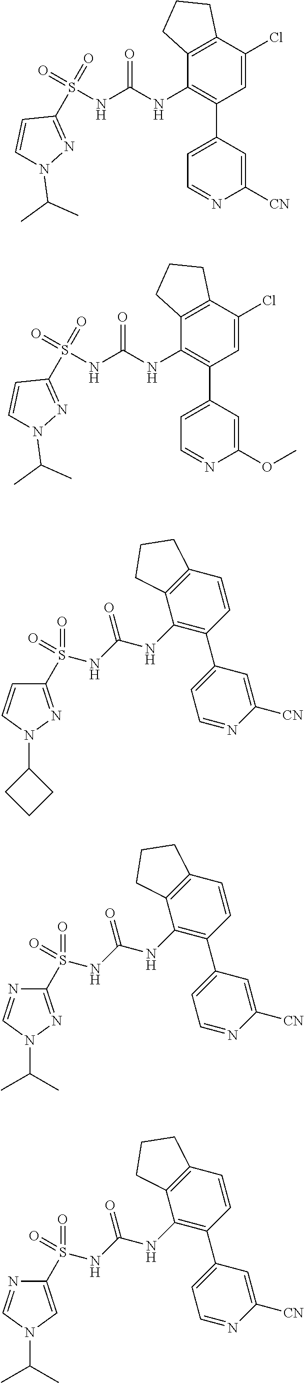

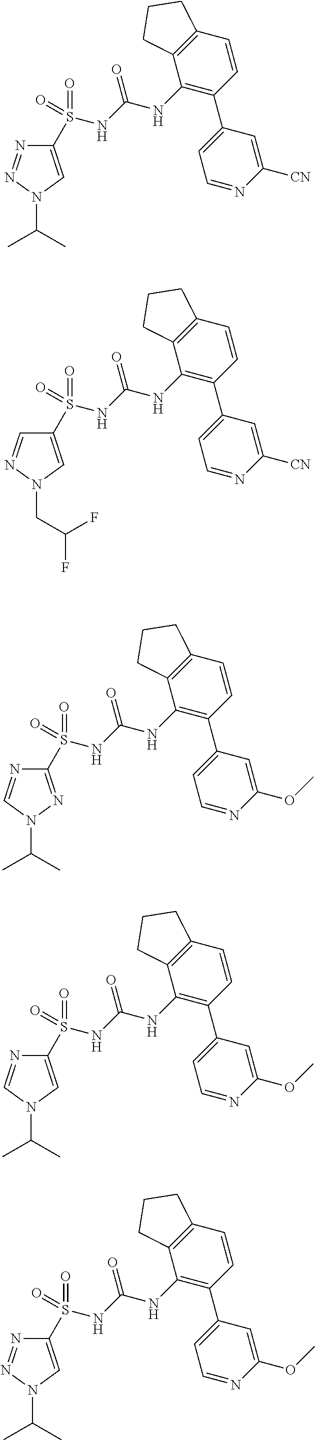

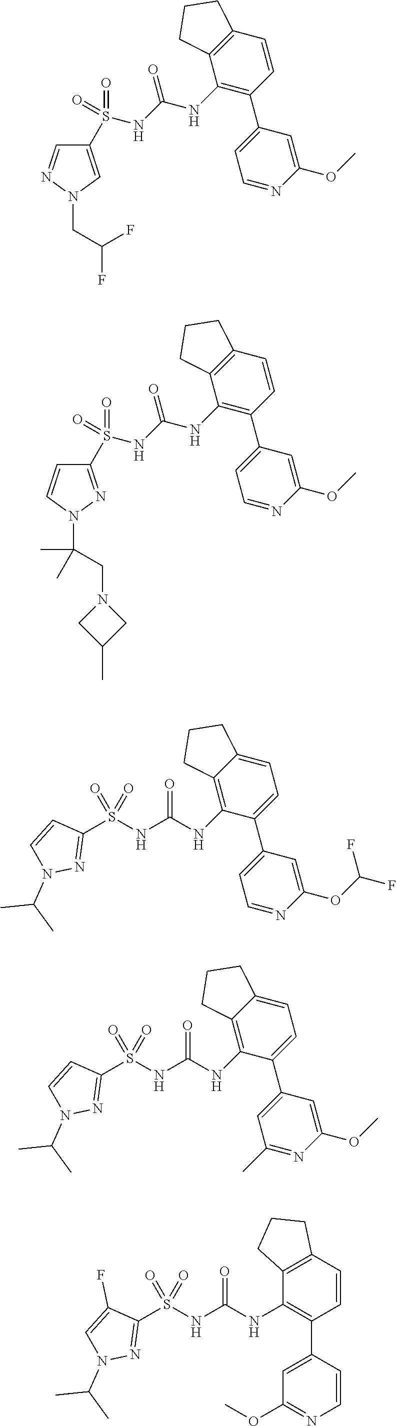

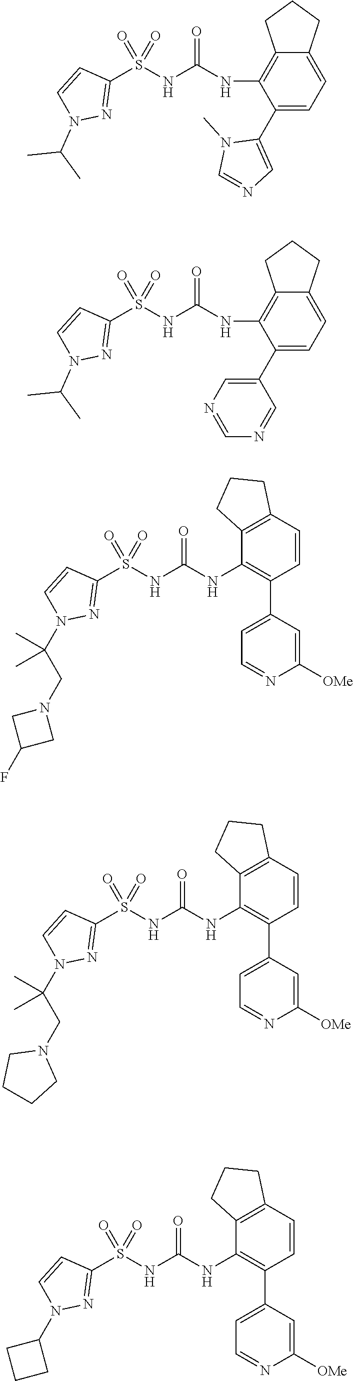

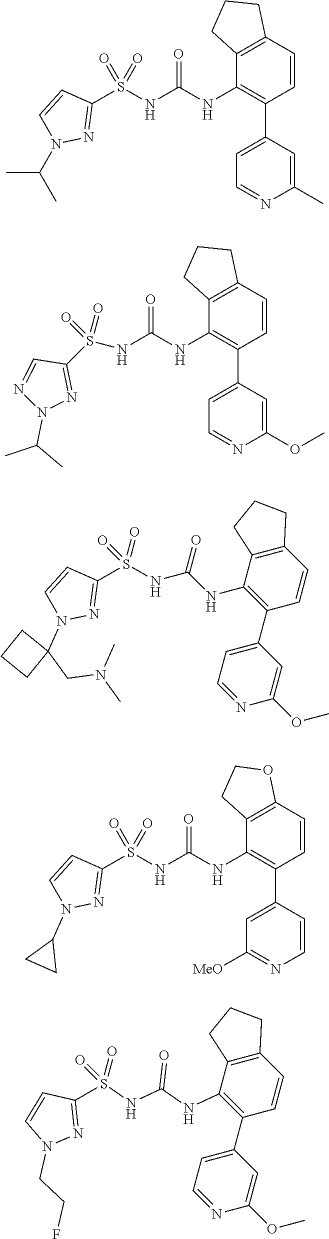

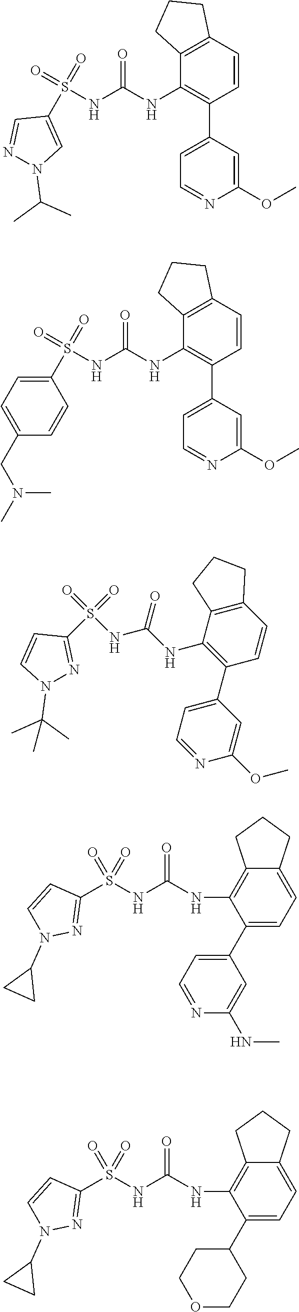

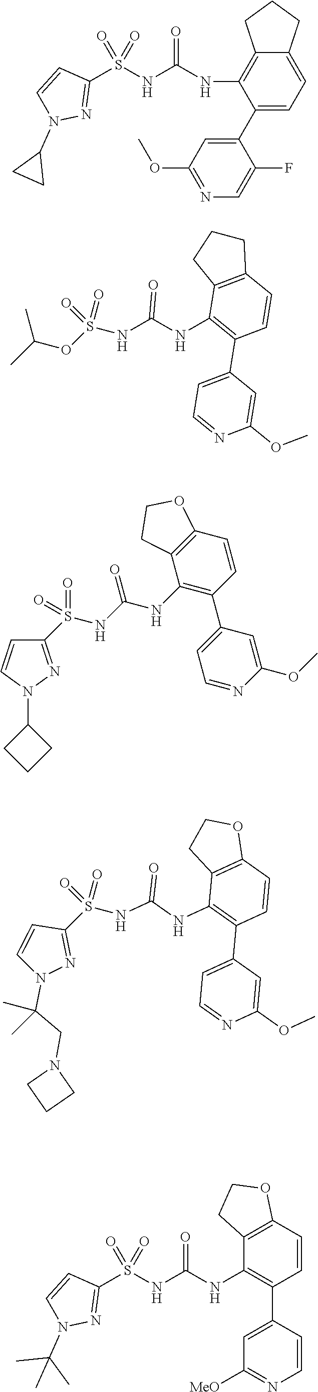

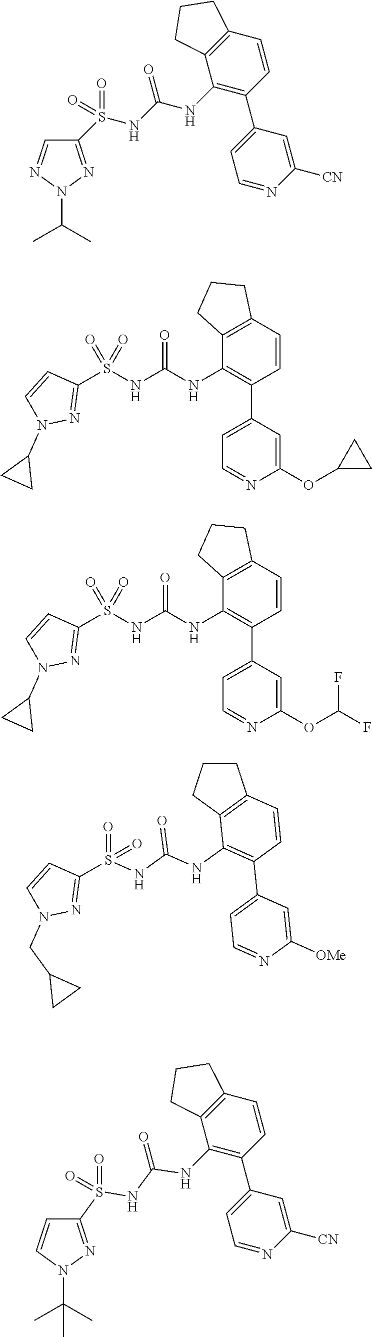

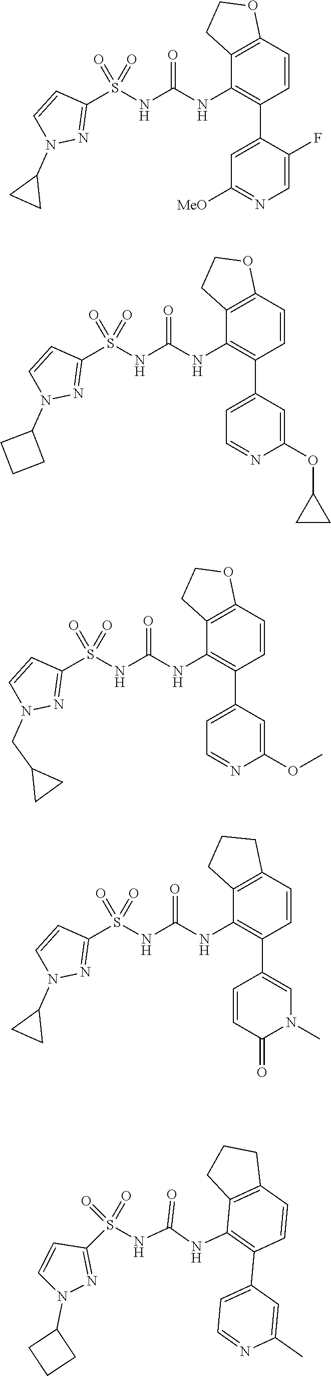 [SMILES: COc1cc(-c2ccc3c(c2NC(=O)NS(=O)(=O)c2ccn(C4CC4)n2)CCO3)c(F)cn1 ; O=C(Nc1c(-c2ccnc(OC3CC3)c2)ccc2c1CCO2)NS(=O)(=O)c1ccn(C2CCC2)n1 ; COc1cc(-c2ccc3c(c2NC(=O)NS(=O)(=O)c2ccn(CC4CC4)n2)CCO3)ccn1 ; Cn1cc(-c2ccc3c(c2NC(=O)NS(=O)(=O)c2ccn(C4CC4)n2)CCC3)ccc1=O ; Cc1cc(-c2ccc3c(c2NC(=O)NS(=O)(=O)c2ccn(C4CCC4)n2)CCC3)ccn1]

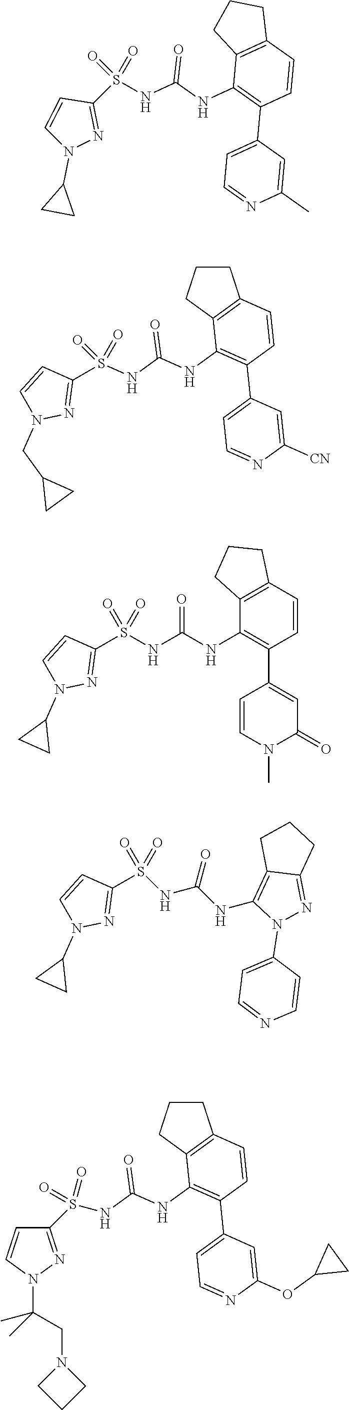

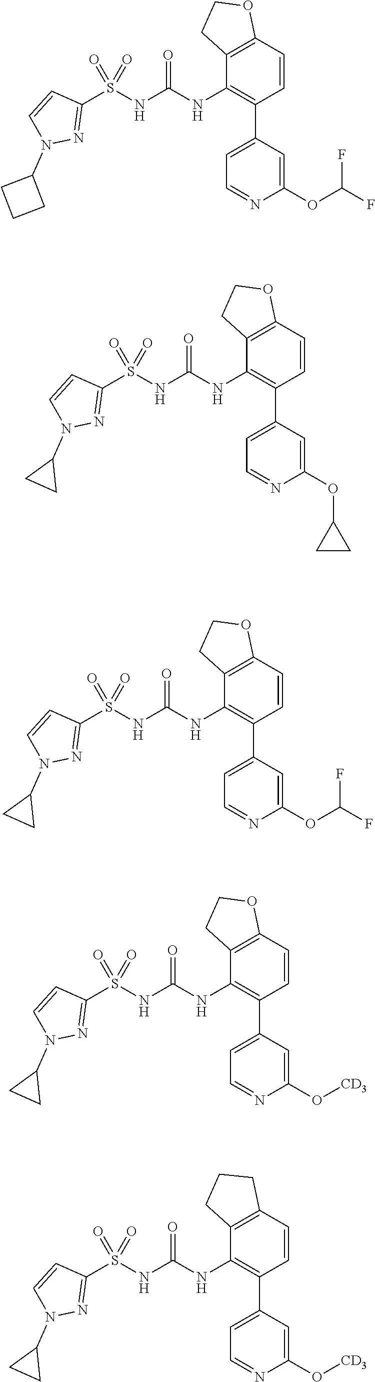 [SMILES: O=C(Nc1c(-c2ccnc(OC(F)F)c2)ccc2c1CCO2)NS(=O)(=O)c1ccn(C2CCC2)n1 ; O=C(Nc1c(-c2ccnc(OC3CC3)c2)ccc2c1CCO2)NS(=O)(=O)c1ccn(C2CC2)n1 ; O=C(Nc1c(-c2ccnc(OC(F)F)c2)ccc2c1CCO2)NS(=O)(=O)c1ccn(C2CC2)n1 ; [2H]C([2H])([2H])Oc1cc(-c2ccc3c(c2NC(=O)NS(=O)(=O)c2ccn(C4CC4)n2)CCO3)ccn1 ; [2H]C([2H])([2H])Oc1cc(-c2ccc3c(c2NC(=O)NS(=O)(=O)c2ccn(C4CC4)n2)CCC3)ccn1]

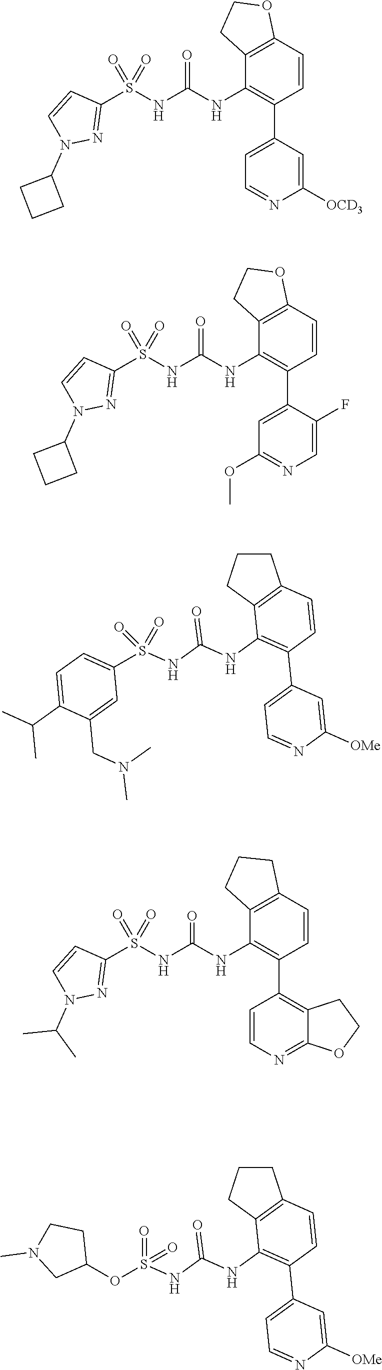

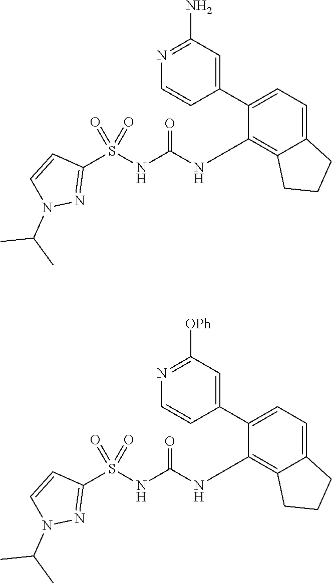 [SMILES: CC(C)n1ccc(S(=O)(=O)NC(=O)Nc2c(-c3ccnc(N)c3)ccc3c2CCC3)n1 ; CC(C)n1ccc(S(=O)(=O)NC(=O)Nc2c(-c3ccnc(Oc4ccccc4)c3)ccc3c2CCC3)n1]

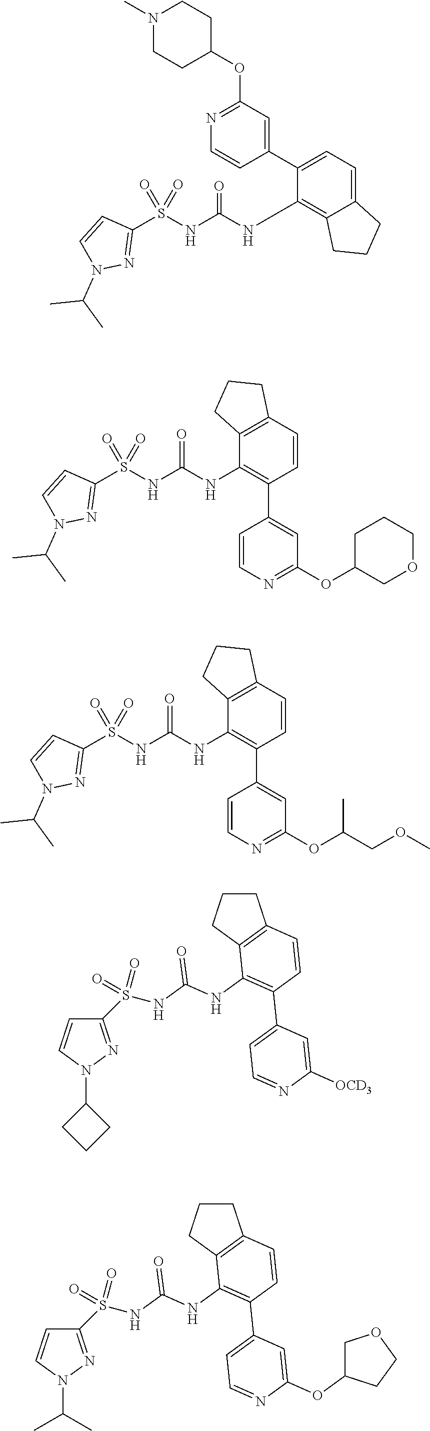

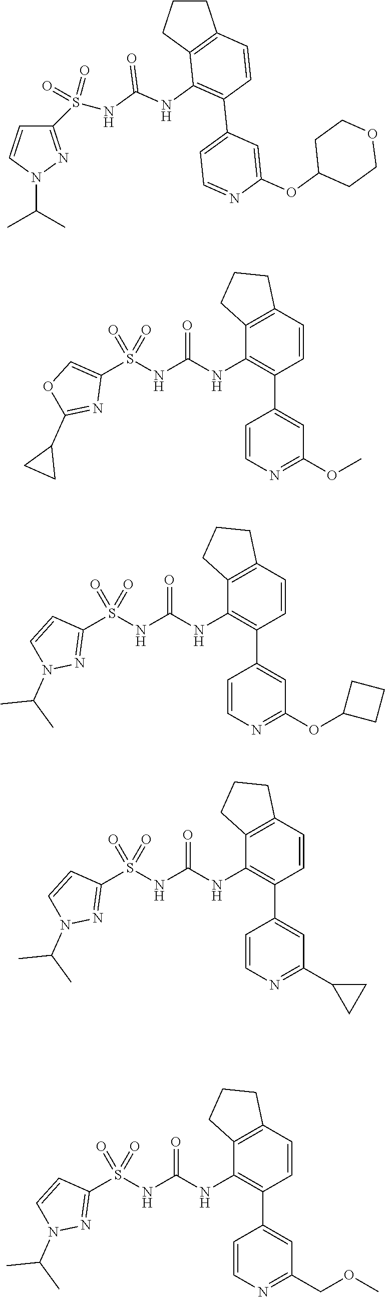

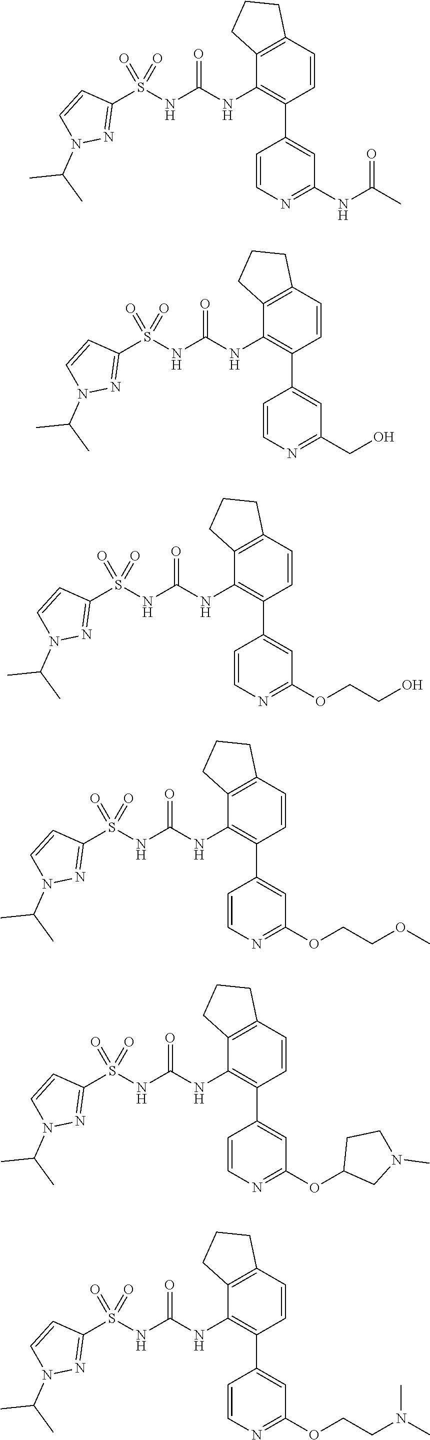

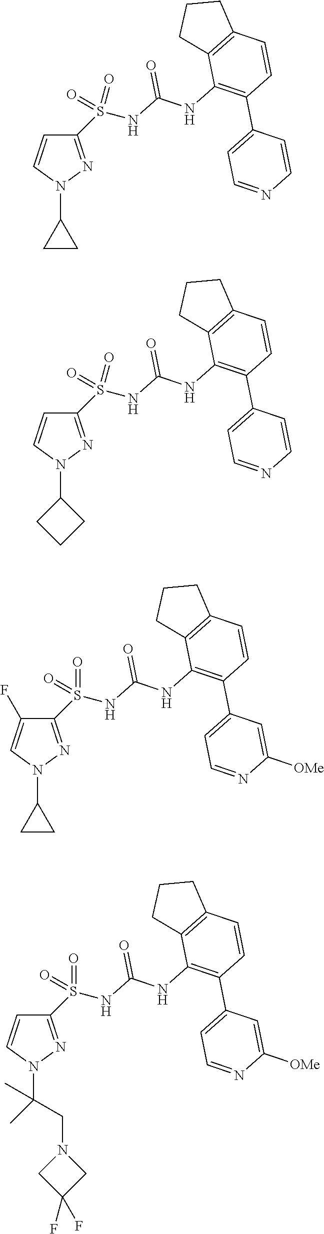 [SMILES: O=C(Nc1c(-c2ccncc2)ccc2c1CCC2)NS(=O)(=O)c1ccn(C2CC2)n1 ; O=C(Nc1c(-c2ccncc2)ccc2c1CCC2)NS(=O)(=O)c1ccn(C2CCC2)n1 ; COc1cc(-c2ccc3c(c2NC(=O)NS(=O)(=O)c2nn(C4CC4)cc2F)CCC3)ccn1 ; COc1cc(-c2ccc3c(c2NC(=O)NS(=O)(=O)c2ccn(C(C)(C)CN4CC(F)(F)C4)n2)CCC3)ccn1]

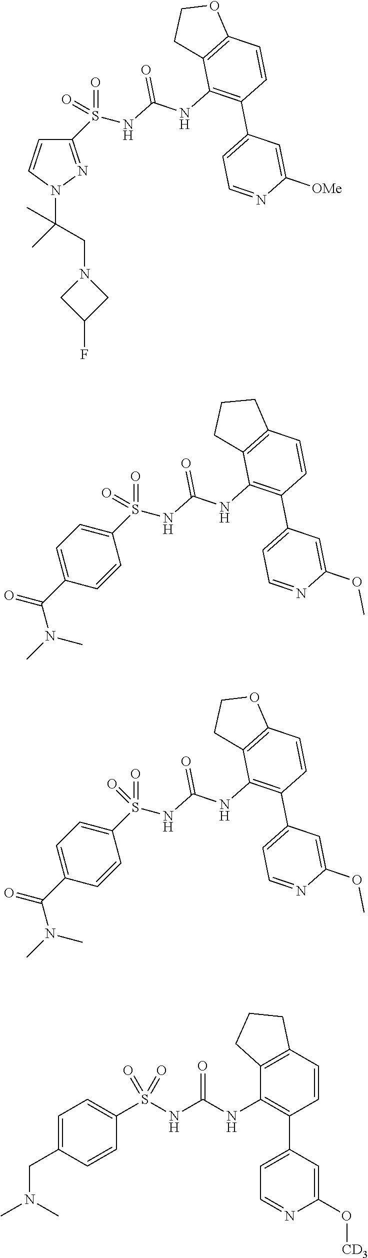

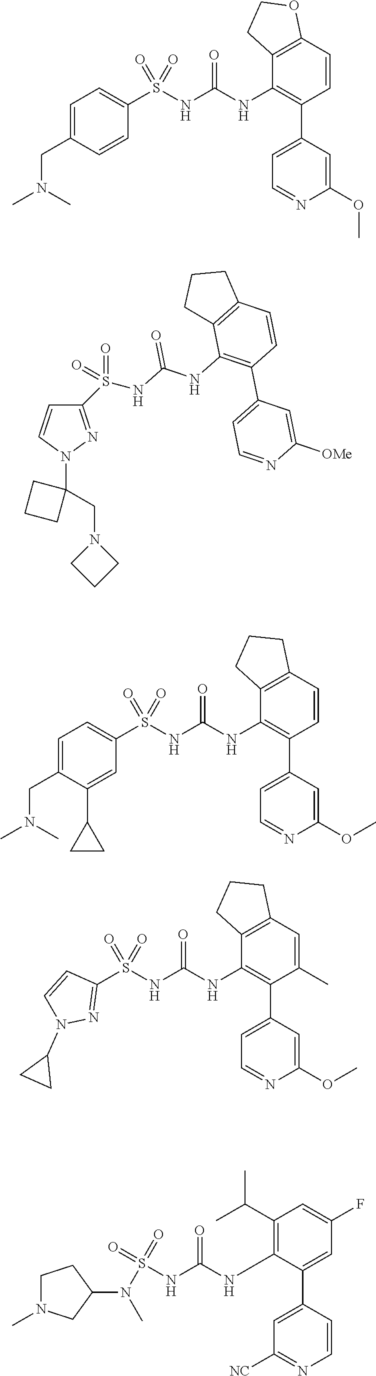

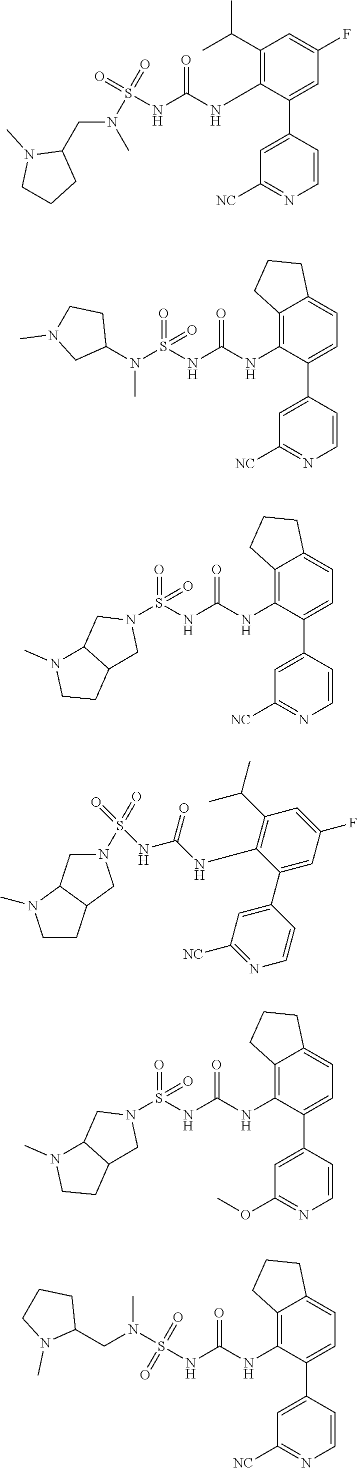

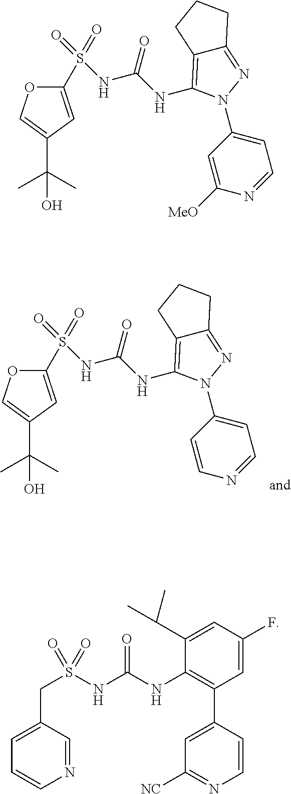 [SMILES: COc1cc(-n2nc3c(c2NC(=O)NS(=O)(=O)c2cc(C(C)(C)O)co2)CCC3)ccn1 ; CC(C)(O)c1coc(S(=O)(=O)NC(=O)Nc2c3c(nn2-c2ccncc2)CCC3)c1 ; CC(C)c1cc(F)cc(-c2ccnc(C#N)c2)c1NC(=O)NS(=O)(=O)Cc1cccnc1]

A third aspect of the invention provides a pharmaceutically acceptable salt, solvate or prodrug of any compound of the first or second aspect of the invention.

The compounds of the present invention can be used both in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid-addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4th Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention is a topical pharmaceutical composition. For example, the topical pharmaceutical composition may be a dermal pharmaceutical composition or an ocular pharmaceutical composition.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptoms, the amelioration or palliation of the condition/symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL1β and IL18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing of an individual having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the present invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481:278-286, 2012).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur. J. Immunol., 40: 595-653, 2010). In particular, NLRP3 mutations have been found to be responsible for a set of rare autoinflammatory diseases known as CAPS (Ozaki et al., J. Inflammation Research, 8:15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type-1 diabetes (T1D), psoriasis, rheumatoid arthritis (RA), Behcet's disease, Schnitzler syndrome, macrophage activation syndrome (Masters Clin. Immunol. 2013; Braddock et al. Nat. Rev. Drug Disc. 2004 3: 1-10; Inoue et al., Immunology 139: 11-18, Coll et al. Nat. Med. 2015 21(3):248-55; and Scott et al. Clin. Exp. Rheumatol 2016 34(1): 88-93), systemic lupus erythematosus (Lu et al. J Immunol. 2017 198(3): 1119-29), and systemic sclerosis (Artlett et al. Arthritis Rheum. 2011; 63(11): 3563-74). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 2017 61: 306-316), intracranial aneurysms (Zhang et al. J. Stroke & Cerebrovascular Dis. 2015 24; 5: 972-979), and traumatic brain injury (Ismael et al. J Neurotrauma. 2018 Jan. 2). NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (Mridha et al. J Hepatol. 2017 66(5): 1037-46). A role for NLRP3 via IL-1β has also been suggested in atherosclerosis, myocardial infarction (van Hout et al. Eur. Heart J 2017 38(11): 828-36), heart failure (Sano et al. JAM. Coll. Cardiol. 2018 71(8): 875-66), aortic aneurysm and dissection (Wu et al. Arterioscler. Thromb. Vasc. Biol. 2017 37(4): 694-706), and other cardiovascular events (Ridker et al, N Engl J Med., doi: 10.1056/NEJM0a1707914, 2017). Other diseases in which NLRP3 has been shown to be involved include: ocular diseases such as both wet and dry age-related macular degeneration (Doyle et al., Nature Medicine, 18: 791-798, 2012 and Tarallo et al. Cell 2012 149 (4): 847-59), diabetic retinopathy (Loukovaara et al. Acta Ophthalmol. 2017; 95(8): 803-808) and optic nerve damage (Puyang et al. Sci Rep. 2016 Feb. 19; 6:20998); liver diseases including non-alcoholic steatohepatitis (NASH) (Henao-Meija et al., Nature, 482: 179-185, 2012); inflammatory reactions in the lung and skin (Primiano et al. J Immunol. 2016 197(6): 2421-33) including contact hypersensitivity (such as bullous pemphigoid (Fang et al. J Dermatol Sci. 2016; 83(2): 116-23)), atopic dermatitis (Niebuhr et al. Allergy 2014 69(8): 1058-67), Hidradenitis suppurativa (Alikhan et al. 2009 J Am Acad Dermatol 60(4): 53961), acne vulgaris (Qin et al. J Invest. Dermatol. 2014 134(2): 381-88), and sarcoidosis (Jager et al. Am J Respir Crit Care Med 2015 191: A5816); inflammatory reactions in the joints (Braddock et al., Nat. Rev. Drug Disc., 3: 1-10, 2004); amyotrophic lateral sclerosis (Gugliandolo et al. Inflammation 2018 41(1): 93-103); cystic fibrosis (Iannitti et al. Nat. Commun. 2016 7: 10791); stroke (Walsh et al., Nature Reviews, 15: 84-97, 2014); chronic kidney disease (Granata et al. PLoS One 2015 10(3): e0122272); and inflammatory bowel diseases including ulcerative colitis and Crohn's disease (Braddock et al., Nat. Rev. Drug Disc., 3: 1-10, 2004, Neudecker et al. J Exp. Med. 2017 214(6): 1737-52, and Lazaridis et al. Dig. Dis. Sci. 2017 62(9): 2348-56). The NLRP3 inflammasome has been found to be activated in response to oxidative stress, and UVB irradiation (Schroder et al., Science, 327: 296-300, 2010). NLRP3 has also been shown to be involved in inflammatory hyperalgesia (Dolunay et al., Inflammation, 40: 366-386, 2017).

The inflammasome, and NLRP3 specifically, has also been proposed as a target for modulation by various pathogens including viruses such as DNA viruses (Amsler et al., Future Virol. (2013) 8(4), 357-370).

NLRP3 has also been implicated in the pathogenesis of many cancers (Menu et al., Clinical and Experimental Immunology 166: 1-15, 2011; and Masters Clin. Immunol. 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al. Lancet, S0140-6736(17)32247-X, 2017). Inhibition of the NLRP3 inflammasome or IL-1β has also been shown to inhibit the proliferation and migration of lung cancer cells in vitro (Wang et al. Oncol Rep. 2016; 35(4): 2053-64). A role for the NLRP3 inflammasome has been suggested in myelodysplastic syndromes (Basiorka et al. Blood. 2016 Dec. 22; 128(25):2960-2975) and also in the carcinogenesis of various other cancers including glioma (Li et al. Am J Cancer Res. 2015; 5(1): 442-449), inflammation-induced tumours (Allen et al. J Exp Med. 2010; 207 (5): 1045-56 and Hu et al. PNAS. 2010; 107(50): 21635-40), multiple myeloma (Li et al. Hematology 2016 21(3): 144-51), and squamous cell carcinoma of the head and neck (Huang et al. J Exp Clin Cancer Res. 2017 2; 36(1): 116). Activation of the NLRP3 inflammasome has also been shown to mediate chemoresistance of tumour cells to 5-Fluorouracil (Feng et al. J Exp Clin Cancer Res. 2017 21; 36(1): 81), and activation of NLRP3 inflammasome in peripheral nerve contributes to chemotherapy-induced neuropathic pain (Jia et al. Mol Pain. 2017; 13: 1-11).

NLRP3 has also been shown to be required for the efficient control of viral, bacterial, fungal, and helminth pathogen infections (Strowig et al., Nature, 481:278-286, 2012). Accordingly, examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;
(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barré syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behçet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler syndrome, macrophage activation syndrome, Blau syndrome, vitiligo or vulvodynia;
(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;
(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr Virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections;
(v) central nervous system diseases such as Parkinson's disease, Alzheimer's disease, dementia, motor neuron disease, Huntington's disease, cerebral malaria, brain injury from pneumococcal meningitis, intracranial aneurysms, traumatic brain injury, and amyotrophic lateral sclerosis;
(vi) metabolic diseases such as type 2 diabetes (T2D), atherosclerosis, obesity, gout, and pseudo-gout;
(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, embolism, aneurysms including abdominal aortic aneurysm, and pericarditis including Dressler's syndrome;
(viii) respiratory diseases including chronic obstructive pulmonary disorder (COPD), asthma such as allergic asthma and steroid-resistant asthma, asbestosis, silicosis, nanoparticle induced inflammation, cystic fibrosis and idiopathic pulmonary fibrosis;
(ix) liver diseases including non-alcoholic fatty liver disease (NAFLD) and non-alcoholic steatohepatitis (NASH) including advanced fibrosis stages F3 and F4, alcoholic fatty liver disease (AFLD), and alcoholic steatohepatitis (ASH);
(x) renal diseases including chronic kidney disease, oxalate nephropathy, nephrocalcinosis, glomerulonephritis, and diabetic nephropathy;
(xi) ocular diseases including those of the ocular epithelium, age-related macular degeneration (AMD) (dry and wet), uveitis, corneal infection, diabetic retinopathy, optic nerve damage, dry eye, and glaucoma;
(xii) skin diseases including dermatitis such as contact dermatitis and atopic dermatitis, contact hypersensitivity, sunburn, skin lesions, hidradenitis suppurativa (HS), other cyst-causing skin diseases, and acne conglobata;
(xiii) lymphatic conditions such as lymphangitis and Castleman's disease;
(xiv) psychological disorders such as depression and psychological stress;
(xv) graft versus host disease;
(xvi) allodynia including mechanical allodynia; and
(xvii) any disease where an individual has been determined to carry a germline or somatic non-silent mutation in NLRP3.

In one embodiment, the disease, disorder or condition is selected from:

(i) cancer;
(ii) an infection;
(iii) a central nervous system disease;
(iv) a cardiovascular disease;
(v) a liver disease;
(vi) an ocular diseases; or
(vii) a skin disease.

More typically, the disease, disorder or condition is selected from:

(i) cancer;
(ii) an infection;
(iii) a central nervous system disease; or
(iv) a cardiovascular disease.

In one embodiment, the disease, disorder or condition is selected from:

(i) acne conglobata;
(ii) atopic dermatitis;
(iii) Alzheimer's disease;
(iv) amyotrophic lateral sclerosis;
(v) age-related macular degeneration (AMD);
(vi) anaplastic thyroid cancer;
(vii) cryopyrin-associated periodic syndromes (CAPS);
(viii) contact dermatitis;
(ix) cystic fibrosis;
(x) congestive heart failure;
(xi) chronic kidney disease;
(xii) Crohn's disease;
(xiii) familial cold autoinflammatory syndrome (FCAS);
(xiv) Huntington's disease;
(xv) heart failure;
(xvi) heart failure with preserved ejection fraction;
(xvii) ischemic reperfusion injury;
(xviii) juvenile idiopathic arthritis;
(xix) myocardial infarction;
(xx) macrophage activation syndrome;
(xxi) myelodysplastic syndrome;
(xxii) multiple myeloma;
(xxiii) motor neuron disease;
(xxiv) multiple sclerosis;
(xxv) Muckle-Wells syndrome;
(xxvi) non-alcoholic steatohepatitis (NASH);
(xxvii) neonatal-onset multisystem inflammatory disease (NOMID);
(xxviii) Parkinson's disease;
(xxix) systemic juvenile idiopathic arthritis;
(xxx) systemic lupus erythematosus;
(xxxi) traumatic brain injury;
(xxxii) transient ischemic attack; and
(xxxiii) ulcerative colitis.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;
(vi) a vascular condition such as atherosclerosis, Behcet's disease, vasculitides, or wegener's granulomatosis;
(vii) an autoimmune condition such as systemic lupus erythematosus, Sjogren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis, Mycobacterium avium intracellulare, Pneumocystis carinii pneumonia, orchitis/epidydimitis, legionella, Lyme disease, influenza A, epstein-barr virus, viral encephalitis/aseptic meningitis, or pelvic inflammatory disease;
(xi) a renal condition such as mesangial proliferative glomerulonephritis, nephrotic syndrome, nephritis, glomerular nephritis, acute renal failure, uremia, or nephritic syndrome;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH) or primary biliary cirrhosis;
(xv) a cancer, including those cancers listed above;
(xvi) a burn, wound, trauma, haemorrhage or stroke;
(xvii) radiation exposure; and/or
(xviii) obesity; and/or
(xix) pain such as inflammatory hyperalgesia.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

In one embodiment of the fifth, sixth, seventh, eighth, ninth or tenth aspect of the present invention, the disease, disorder or condition is not a disease or disorder mediated by NFκB. In one embodiment of the fifth, sixth, seventh, eighth, ninth or tenth aspect of the present invention, the disease, disorder or condition is not rheumatoid arthritis, osteoarthritis, an autoimmune disease, psoriasis, asthma, a cardiovascular disease, an acute coronary syndrome, atherosclerosis, myocardial infarction, unstable angina, congestive heart failure, Alzheimer's disease, multiple sclerosis, cancer, type II diabetes, metabolic syndrome X, inflammatory bowel disease, systemic lupus erythematosus, Grave's disease, myasthenia gravis, insulin resistance, autoimmune hemolytic anemia, scleroderma with anticollagen antibodies, pernicious anemia, or diabetes mellitus. In one embodiment of the fifth, sixth, seventh, eighth, ninth or tenth aspect of the present invention, the disease, disorder or condition is not inflammatory bowel disease.

In one embodiment of the fifth, sixth, seventh, eighth, ninth or tenth aspect of the present invention, the treatment or prevention comprises topically administering a compound of the first or second aspect, or a pharmaceutically acceptable salt, solvate or prodrug of the third aspect, or a pharmaceutical composition of the fourth aspect. For example, the disease, disorder or condition may be a skin disease or condition, wherein the treatment or prevention comprises topically administering a compound of the first or second aspect, or a pharmaceutically acceptable salt, solvate or prodrug of the third aspect, or a pharmaceutical composition of the fourth aspect to the skin. Alternatively, the disease, disorder or condition may be an ocular disease or condition, wherein the treatment or prevention comprises topically administering a compound of the first or second aspect, or a pharmaceutically acceptable salt, solvate or prodrug of the third aspect, or a pharmaceutical composition of the fourth aspect to the eye.

In one embodiment, where the treatment or prevention comprises topically administering a compound of the first or second aspect, or a pharmaceutically acceptable salt, solvate or prodrug of the third aspect, or a pharmaceutical composition of the fourth aspect of the invention, one or more further active agents may be co-administered. The one or more further active agents may also be topically administered, or may be administered via a non-topical route. Typically, the one or more further active agents are also topically administered. For example, where the pharmaceutical composition of the fourth aspect of the invention is a topical pharmaceutical composition, the pharmaceutical composition may further comprise one or more further active agents.

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;
(ii) antibodies;
(iii) alkylating agents;
(iv) anti-metabolites;
(v) anti-angiogenic agents;
(vi) plant alkaloids and/or terpenoids;
(vii) topoisomerase inhibitors;
(viii) mTOR inhibitors;
(ix) stilbenoids;
(x) STING agonists;
(xi) cancer vaccines;
(xii) immunomodulatory agents;
(xiii) antibiotics;
(xiv) anti-fungal agents;
(xv) anti-helminthic agents; and/or
(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

In some embodiments, the one or more chemotherapeutic agents are selected from abiraterone acetate, altretamine, amsacrine, anhydrovinblastine, auristatin, azathioprine, adriamycin, bexarotene, bicalutamide, BMS 184476, bleomycin, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, leucovorin, lonidamine, lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulphonamide, prednimustine, procarbazine, rhizoxin, sertenef, streptozocin, stramustine phosphate, tretinoin, tasonermin, taxol, topotecan, tamoxifen, teniposide, taxane, tegafur/uracil, vincristine, vinblastine, vinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), interferon alpha, interferon beta, interferon gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment), and/or cytokines (including interleukins, such as interleukin-2 (IL-2), or IL-10).

In some embodiments, the one or more antibodies may comprise one or more monoclonal antibodies. In some embodiments, the one or more antibodies are selected from abciximab, adalimumab, alemtuzumab, atlizumab, basiliximab, belimumab, bevacizumab, bretuximab vedotin, canakinumab, cetuximab, ceertolizumab pegol, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumuab, ranibizumab, rituximab, tocilizumab, tositumomab, and/or trastuzumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from endostatin, angiogenin inhibitors, angiostatin, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR⁶⁷) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides, such as cAMP, cGMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃).

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAGS), MHC class I, MHC class II, 4-1BB, 4-1BBL, OX40, OX40L, GITR, GITRL, CD27, CD70, TNFRSF25, TL1A, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR4, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCB024360, galunisertib, ulocuplumab, BKT140, bavituximab, CC-90002, bevacizumab, and/or MNRP1685 A.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents, anti-inflammatory agents (including nonsteroidal anti-inflammatory agents), anti-psoriatic agents (including anthralin and its derivatives), vitamins and vitamin-derivatives (including retinoinds, and VDR receptor ligands), corticosteroids, ion channel blockers (including potassium channel blockers), immune system regulators (including cyclosporin, FK 506, and glucocorticoids), lutenizing hormone releasing hormone agonists (such as leuprolidine, goserelin, triptorelin, histrelin, bicalutamide, flutamide and/or nilutamide), and/or hormones (including estrogen).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disorder, disease or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES—COMPOUND SYNTHESIS

All solvents, reagents and compounds were purchased and used without further purification unless stated otherwise.

### Abbreviations

2-MeTHF 2-methyltetrahydrofuran
Ac₂O acetic anhydride
AcOH acetic acid
aq aqueous
Boc tert-butyloxycarbonyl
br broad
Cbz carboxybenzyl
CDI 1,1-carbonyl-diimidazole
conc concentrated
d doublet
DAB CO 1,4-diazabicyclo[2.2.2]octane
DCE 1,2-dichloroethane, also called ethylene dichloride
DCM dichloromethane
DIPEA N,N-diisopropylethylamine, also called Hünig's base
DMA dimethylacetamide
DMAP 4-dimethylaminopyridine, also called N,N-dimethylpyridin-4-amine
DME dimethoxyethane
DMF N,N-dimethylformamide
DMSO dimethyl sulfoxide
eq or equiv equivalent
(ES+) electrospray ionization, positive mode
Et ethyl
EtOAc ethyl acetate
EtOH ethanol
h hour(s)
HATU 1-[bis(dimethylamino)methylene]-1H-1,2,3-triazolo[4,5-b]pyridinium 3-oxid hexafluorophosphate
HPLC high performance liquid chromatography
LC liquid chromatography
m multiplet
m-CPBA 3-chloroperoxybenzoic acid
Me methyl
MeCN acetonitrile
MeOH methanol
(M+H)+ protonated molecular ion
MHz megahertz
min minute(s)
MS mass spectrometry
Ms mesyl, also called methanesulfonyl
MSCl mesyl chloride, also called methanesulfonyl chloride
MTBE methyl tert-butyl ether, also called tert-butyl methyl ether
m/z mass-to-charge ratio
NaOtBu sodium tert-butoxide
NBS 1-bromopyrrolidine-2,5-dione, also called N-bromosuccinimide
NCS 1-chloropyrrolidine-2,5-dione, also called N-chlorosuccinimide
NMP N-methylpyrrolidine
NMR nuclear magnetic resonance (spectroscopy)
Pd(dba)₃ tris(dibenzylideneacetone)dipalladium(0)
Pd(dppf)Cl₂ [1,1′-bis(diphenylphosphino)ferrocene] dichloropalladium(II)
PE petroleum ether
Ph phenyl
PMB p-methoxybenzyl, also called 4-methoxybenzyl
prep-HPLC preparative high performance liquid chromatography
prep-TLC preparative thin layer chromatography
PTSA p-toluenesulfonic acid
q quartet
RP reversed phase
RT room temperature
s singlet
Sept septuplet
sat saturated
SCX solid supported cation exchange (resin)
t triplet
T3P propylphosphonic anhydride
TBME tert-butyl methyl ether, also called methyl tert-butyl ether
TEA triethylamine
TFA 2,2,2-trifluoroacetic acid
THF tetrahydrofuran
TLC thin layer chromatography
wt % weight percent or percent by weight

### Experimental Methods

### ¹H NMR Spectroscopy

Nuclear magnetic resonance (NMR) spectra were recorded at 300, 400 or 500 MHz unless stated otherwise; the chemical shifts are reported in parts per million. Spectra were measured at 298 K, unless indicated otherwise, and were referenced relative to the solvent resonance. Spectra were recorded using one of the following machines:
A Bruker Advance III spectrometer at 400 MHz fitted with a BBO 5 mm liquid probe.
A Bruker 400 MHz spectrometer using ICON-NMR, under TopSpin program control.
A Bruker Avance III HD spectrometer at 500 MHz, equipped with a Bruker 5 mm SmartProbe™.
An Agilent VNMRS 300 instrument fitted with a 7.05 Tesla magnet from Oxford instruments, indirect detection probe and direct drive console including PFG module.
An Agilent MercuryPlus 300 instrument fitted with a 7.05 Tesla magnet from Oxford instruments, 4 nuclei auto-switchable probe and Mercury plus console.

### LC-MS Methods

Using SHIMADZU LCMS-2020, Agilent 1200 LC/G1956A MSD and Agilent 1200\G6110A, Agilent 1200 LC & Agilent 6110 MSD. Mobile Phase: A: 0.025% NH₃H₂O in water (v/v); B: acetonitrile. Column: Kinetex EVO C18 2.1×30 mm, 5 μm.

### Reversed Phase HPLC Conditions for the LCMS Analytical Methods

Methods 1a and 1b:

Waters Xselect CSH C18 XP column, 2.5 μm (4.6×30 mm) at 40° C.; flow rate 2.5-4.5 mL min⁻¹ eluted with a water-acetonitrile gradient containing either 0.1% v/v formic acid (Method 1a) or 10 mM ammonium bicarbonate in water (Method 1b) over 4 minutes employing UV detection at 254 nm. Gradient information: 0-3.00 min, ramped from 95% water-5% acetonitrile to 5% water-95% acetonitrile; 3.00-3.01 min, held at 5% water-95% acetonitrile, flow rate increased to 4.5 mL min 1; 3.01-3.50 min, held at 5% water-95% acetonitrile; 3.50-3.60 min, returned to 95% water-5% acetonitrile, flow rate reduced to 3.50 mL min⁻¹; 3.60-3.90 min, held at 95% water-5% acetonitrile; 3.90-4.00 min, held at 95% water-5% acetonitrile, flow rate reduced to 2.5 mL min⁻¹.

### Method 1c:

Agilent 1290 series with UV detector and HP 6130 MSD mass detector using Waters XBridge BEH C18 XP column (2.1×50 mm, 2.5 μm) at 35° C.; flow rate 0.6 mL/min; mobile phase A: ammonium acetate (10 mM); water/MeOH/acetonitrile (900:60:40); mobile phase B: ammonium acetate (10 mM); water/MeOH/acetonitrile (100:540:360); over 4 min employing UV detection at 215 and 238 nm. Gradient information: 0-0.5 min, held at 80% A-20% B; 0.5-2.0 min, ramped from 80% A-20% B to 100% B.

### Reversed Phase HPLC Conditions for the UPLC Analytical Methods

### Methods 2a and 2b:

Waters BEH C18, 1.7 μm, (2.1×30 mm) at 40° C.; flow rate 0.77 mL min⁻¹ eluted with a water-acetonitrile gradient containing either 0.1% v/v formic acid (Method 2a) or 10 mM ammonium bicarbonate in water (Method 2b) over 3 minutes employing UV detection at 254 nm. Gradient information: 0-0.11 min, held at 95% water-5% acetonitrile, flow rate 0.77 mL min⁻¹; 0.11-2.15 min, ramped from 95% water-5% acetonitrile to 5% water-95% acetonitrile; 2.15-2.49 min, held at 5% water-95% acetonitrile, flow rate 0.77 mL min⁻¹; 2.49-2.56 min, returned to 95% water-5% acetonitrile; 2.56-3.00 min, held at 95% water-5% acetonitrile, flow rate reduced to 0.77 mL min⁻¹.

### Preparative Reversed Phase High Performance Liquid Chromatography General Methods

### Method 1 (Acidic Preparation):

Waters X-Select CSH column C18, 5 μm (19×50 mm), flow rate 28 mL/min eluting with a water-acetonitrile gradient containing 0.1% v/v formic acid over 6.5 minutes using UV detection at 254 nm. Gradient information: 0.0-0.2 minutes, 20% acetonitrile; 0.2-5.5 minutes, ramped from 20% acetonitrile to 40% acetonitrile; 5.5-5.6 minutes, ramped from 40% acetonitrile to 95% acetonitrile; 5.6-6.5 minutes, held at 95% acetonitrile.

### Method 2 (Basic Preparation):

Waters X-Bridge Prep column C18, 5 μm (19×50 mm), flow rate 28 mL/min eluting with a 10 mM ammonium bicarbonate-acetonitrile gradient over 6.5 minutes using UV detection at 254 nm. Gradient information: 0.0-0.2 minutes, 10% acetonitrile; 0.2-5.5 minutes, ramped from 10% acetonitrile to 40% acetonitrile; 5.5-5.6 minutes, ramped from 40% acetonitrile to 95% acetonitrile; 5.6-6.5 minutes, held at 95% acetonitrile.

### Method 3:

Phenomenex Gemini column, 10 μm (150×25 mm), flow rate=25 mL/min eluting with a water-acetonitrile gradient containing 0.04% NH₃ at pH 10 over 9 minutes using UV detection at 220 and 254 nm. Gradient information: 0-9 minutes, ramped from 8% to 35% acetonitrile; 9-9.2 minutes, ramped from 35% to 100% acetonitrile; 9.2-15.2 minutes, held at 100% acetonitrile.

Method 4: Revelis C18 reversed-phase 12 g cartridge [carbon loading 18%; surface area 568 m²/g; pore diameter 65 Angstrom; pH (5% slurry) 5.1; average particle size 40 μm], flow rate=30 mL/min eluting with a water-methanol gradient over 35 minutes using UV detection at 215, 235, 254 and 280 nm. Gradient information: 0-5 minutes, held at 0% methanol; 5-30 minutes, ramped from 0% to 70% methanol; 30-30.1 minutes, ramped from 70% to 100% methanol; 30.1-35 minutes, held at 100% methanol.

### Synthesis of Intermediates

### Intermediate P1: 5-((Dimethylamino)methyl)-1-methyl-1H-pyrazole-3-sulfonamide

### Step A: N,N-Bis-(4-methoxybenzyl)-1-methyl-1H-pyrazole-3-sulfonamide

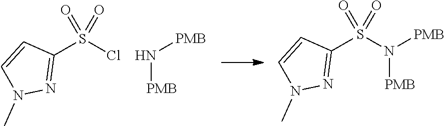

A solution of 1-methyl-1H-pyrazole-3-sulfonyl chloride (13.0 g, 72.0 mmol) in dichloromethane (30 mL) was added slowly to a solution of bis-(4-methoxybenzyl)amine (20 g, 78 mmol) and triethylamine (20 mL, 143 mmol) in dichloromethane (250 mL) cooled in an ice bath. The mixture was stirred for 30 minutes, warmed to room temperature and stirred for 2 hours. The mixture was washed with water (200 mL), hydrochloric acid (aqueous, 1 M, 200 mL) and water (200 mL), then dried (magnesium sulfate), filtered and concentrated in vacua. The residue was triturated with tert-butylmethylether (250 mL), filtered, then purified by chromatography on silica gel (330 g column, 0-60% ethyl acetate/iso-hexane) to afford the title compound (27.66 g, 93%) as a white solid.

¹H NMR (CDCl₃) δ 7.42 (d, 1H), 7.11-7.07 (m, 4H), 6.81-6.77 (m, 4H), 6.65 (d, 1H), 4.33 (s, 4H), 3.99 (s, 3H) and 3.81 (s, 6H).

LCMS m/z 402 (M+H)⁺ (ES⁺).

### Step B: 5-((Dimethylamino)methyl)-N,N-bis(4-methoxybenzyl)-1-methyl-1H-pyrazole-3-sulfonamide

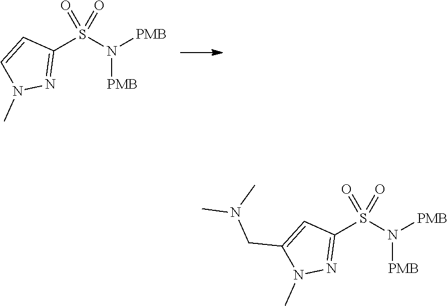

A solution of n-BuLi (2.5 M in hexanes; 4.2 mL, 10.50 mmol) was added drop-wise to a stirred solution of N,N-bis-(4-methoxybenzyl)-1-methyl-1H-pyrazole-3-sulfonamide (4 g, 9.96 mmol) in tetrahydrofuran (60 mL) at −78° C. The reaction was stirred for 1 hour, then N-methyl-N-methylenemethanaminium iodide (4 g, 21.62 mmol) was added. The reaction mixture was left at −78° C. for 2 hours before the reaction was quenched with water (20 mL) and extracted with ethyl acetate (2×20 mL). The organic layer was separated, dried (magnesium sulfate), filtered and concentrated in vacuo. The crude product was purified by chromatography (Companion apparatus, 120 g column, 0-10% methanol/dichloromethane), then loaded onto a further column (SCX, 13 g) in methanol. The column was washed with methanol and then the product was eluted with 0.7 M ammonia in methanol. The resultant mixture was purified further by chromatography on silica (80 g column, 0-5% methanol/dichloromethane) to afford the title compound (1.9 g, 38%) as a colourless oil.

¹H NMR (DMSO-d₆) δ 7.07-7.01 (m, 4H), 6.84-6.78 (m, 4H), 6.58 (s, 1H), 4.21 (s, 4H), 3.89 (s, 3H), 3.72 (s, 6H), 3.47 (s, 2H) and 2.16 (s, 6H).

LCMS m/z 459.8 (M+H)⁺ (ES⁺).

### Step C: 5-((Dimethylamino)methyl)-1-methyl-1H-pyrazole-3-sulfonamide

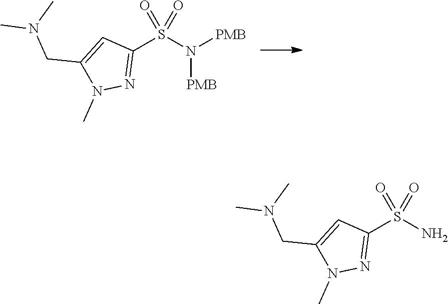

5-((Dimethylamino)methyl)-N,N-bis(4-methoxybenzyl)-1-methyl-1H-pyrazole-3-sulfonamide (891 mg, 1.94 mmol) was dissolved in dichloromethane (3 mL) and trifluoroacetic acid (3 mL) was added. The solution was stirred for 16 hours and then additional trifluoroacetic acid (2 mL) was added. The solution was stirred for another 16 hours before a further aliquot of trifluoroacetic acid (2 mL) was added and the solution stirred for 16 hours. The reaction mixture was concentrated in vacua, suspended in toluene (5 mL) and concentrated again. The crude product was loaded onto a column (SCX; 4 g) in methanol and the column was washed with methanol and then the product was eluted with 0.7 M ammonia in methanol. The resultant mixture was concentrated in vacua to afford the title compound (337 mg, 79%) as a white solid.

¹H NMR (DMSO-d₆) δ 7.36 (br s, 2H), 6.51 (s, 1H), 3.86 (s, 3H), 3.32 (s, 2H) and 2.23 (s, 6H).

LCMS m/z 219.3 (M+H)⁺ (ES⁺).

### Intermediate P2: 5-((Dimethylamino)methyl)-1-isopropyl-1H-pyrazole-3-sulfonamide

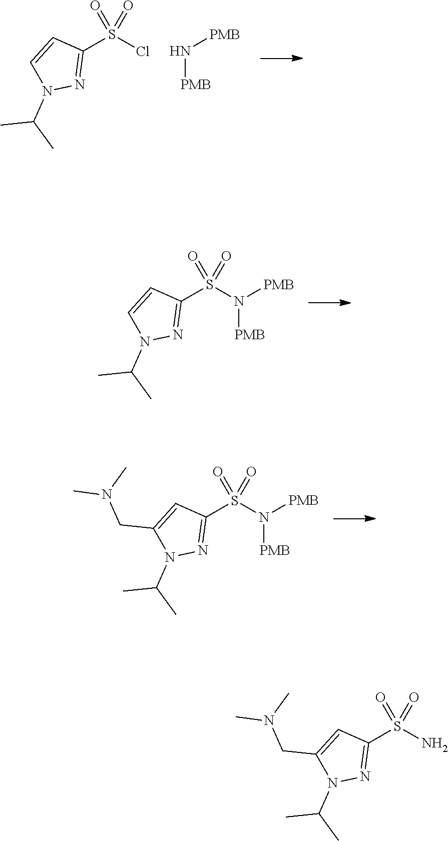

The title compound was prepared according to the procedure for 5-((dimethylamino) methyl)-1-methyl-1H-pyrazole-3-sulfonamide (Intermediate P1) (339 mg, 73%).

¹H NMR (DMSO-d₆) δ 7.35 (s, 2H), 6.45 (s, 1H), 4.78 (sep, 1H), 3.47 (s, 2H), 2.16 (s, 6H) and 1.38 (d, 6H).

### Intermediate P3: (4-(Dimethylamino)pyridin-1-ium-1-carbonyl)((1-isopropyl-1H-pyrazol-3-yl)sulfonyl)amide

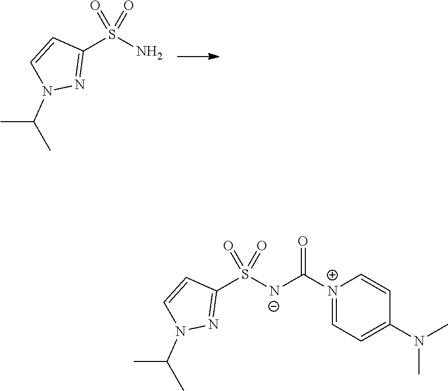 [SMILES: CC(C)n1ccc(S(N)(=O)=O)n1 ; CC(C)n1ccc(S(=O)(=O)[N-]C(=O)[n+]2ccc(N(C)C)cc2)n1]

A solution of 1-isopropyl-1H-pyrazole-3-sulfonamide (712 mg, 3.76 mmol) in acetonitrile (4.4 mL) was treated with N,N-dimethylpyridin-4-amine (919 mg, 7.53 mmol) and the reaction mixture was stirred at room temperature until sulfonamide had dissolved. Diphenyl carbonate (887 mg, 4.14 mmol) was added and the reaction mixture was left for 16 hours at room temperature. The resulting precipitate was separated by filtration, washed with methyl tert-butylether and dried to afford the title compound (776 mg, 61%) as a white solid, which was used without further purification.

¹H NMR (CDCl₃) δ 8.95 (d, J=7.5 Hz, 2H), 7.35 (d, J=2.3 Hz, 1H), 6.83 (d, J=2.3 Hz, 1H), 6.62 (d, J=7.5 Hz, 2H), 4.58-4.43 (m, 1H), 3.24 (s, 6H), 1.42 (d, J=6.7 Hz, 6H).

### Intermediate P4: (4-(Dimethylamino)pyridin-1-ium-1-carbonyl)((5-(dimethylcarbamoyl)-1-methyl-1H-pyrazol-3-yl)sulfonyl)amide

### Step A: 1-Methyl-3-sulfamoyl-1H-pyrazole-5-carboxylic acid, sodium salt

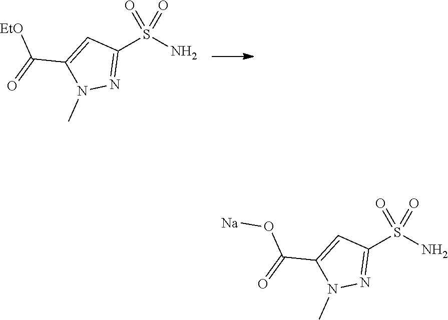

To a suspension of ethyl 1-methyl-3-sulfamoyl-1H-pyrazole-5-carboxylate (3 g, 12.86 mmol) in ethanol (60 mL) was added a solution of sodium hydroxide (2.0 M, 13.5 mL) and the mixture was stirred at room temperature for 2 hours. The resulting precipitate was filtered off, washed with ethanol and dried to afford the title compound (2.92 g, 99%) as a white solid.

¹H NMR (D₂O) δ6.79 (s, 1H) and 4.01 (s, 3H).

### Step B: N,N,1-Trimethyl-3-sulfamoyl-1H-pyrazole-5-carboxamide

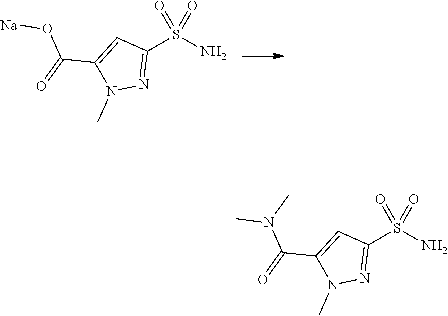

To a mixture of 1-methyl-3-sulfamoyl-1H-pyrazole-5-carboxylic acid, sodium salt (2.38 g, 10.48 mmol) was added T3P (50% in ethyl acetate, 12.47 ml, 20.95 mmol) and N,N-diisopropylethylamine (Hunig's Base, 3.66 ml, 20.95 mmol) in tetrahydrofuran (50 mL). A solution of 2.0 M dimethylamine in THF (15.71 ml, 31.4 mmol) was added and the reaction stirred for 20 hours before being quenched with saturated aqueous ammonium chloride (10 mL) and extracted with ethyl acetate (3×20 ml). The combined extracts were dried (magnesium sulfate), filtered and evaporated in vacuo to afford a yellow gum. The crude product was triturated in dichloromethane (20 mL) and filtered to obtain the title compound (900 mg) as a white solid. The mother layers were evaporated, dissolved in dichloromethane/methanol and purified by chromatography (Companion apparatus, 40 g column, 0-10% methanol/dichloromethane with product eluting at ˜5% methanol) to afford a further batch of the title compound (457 mg) as a white solid. The solids were combined to afford the title compound (1.36 g, 55%).

¹H NMR (DMSO-d₆) δ 7.50 (s, 2H), 6.82 (s, 1H), 3.90 (s, 3H), 3.03 (s, 3H) and 3.01 (s, 3H).

LCMS m/z 233.0 (M+H)⁺ (ES⁺).

### Step C: (4-(Dimethylamino)pyridin-1-ium-1-carbonyl)((5-(dimethylcarbamoyl)-1-methyl-1H-pyrazol-3-yl)sulfonyl)amide

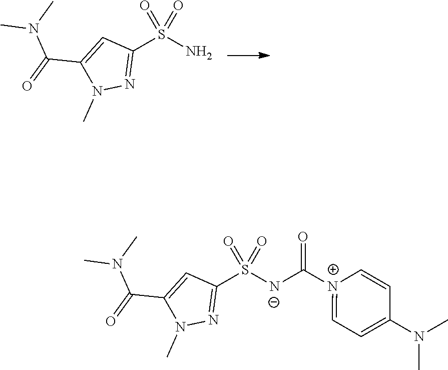

A solution of N,N,1-trimethyl-3-sulfamoyl-1H-pyrazole-5-carboxamide (459 mg, 1.976 mmol) in acetonitrile (2.3 mL) was treated with N,N-dimethylpyridin-4-amine (483 mg, 3.95 mmol) and the reaction mixture was stirred at room temperature until the sulfonamide had dissolved. Diphenyl carbonate (466 mg, 2.174 mmol) was added and the reaction mixture was left for 16 hours at room temperature. The resulting precipitate was separated by filtration, washed with acetonitrile and dried to afford the title compound (578 mg, 77%) which was used in the next step without further purification.

¹H NMR (DMSO-d6) δ 8.77-8.73 (m, 2H), 7.02-6.98 (m, 2H), 6.83 (s, 1H), 3.85 (s, 3H), 3.26 (s, 6H), 3.05 (s, 3H), 3.00 (s, 3H).

### Intermediate P5: (4-(Dimethylamino)pyridin-1-ium-1-carbonyl)((5-(2-methoxypropan-2-yl)-1-methyl-1H-pyrazol-3-yl)sulfonyl)amide

### Step A: Ethyl 3-(N,N-bis(4-methoxybenzyl)sulfamoyl)-1-methyl-1H-pyrazole-5-carboxylate

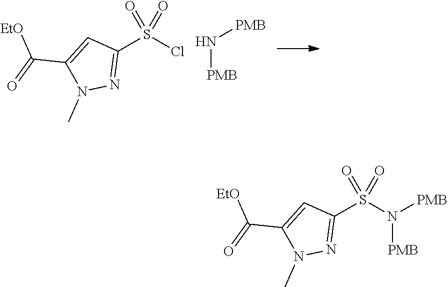

Ethyl 3-(chlorosulfonyl)-1-methyl-1H-pyrazole-5-carboxylate (9.2 g, 36.4 mmol) was added drop-wise to a solution of bis(4-methoxybenzyl)amine (9.4 g, 36.5 mmol) and triethylamine (10 mL, 71.7 mmol) in dichloromethane (200 mL) cooled in an ice bath. The resulting mixture was stirred for 30 minutes, warmed to room temperature and stirred for 90 minutes before being washed with water (200 mL), aqueous hydrochloric acid (1 M, 200 mL), water (200 mL), dried (magnesium sulfate), filtered and evaporated to give a yellow oil. This was purified by chromatography on silica gel (220 g column, 0-60% ethyl acetate/iso-hexane) to afford the title compound (15.9 g, 91%) as a white solid.

¹H NMR (DMSO-d₆) δ 7.19-7.00 (m, 5H), 6.85-6.77 (m, 4H), 4.33 (q, 2H), 4.25 (s, 4H), 4.15 (s, 3H), 3.71 (s, 6H) and 1.33 (t, 3H).

LCMS m/z 496.4 (M+Na)⁺ (ES⁺).

### Step B: 5-(2-Hydroxypropan-2-yl)-N,N-bis(4-methoxybenzyl)-1-methyl-1H-pyrazole-3-sulfonamide

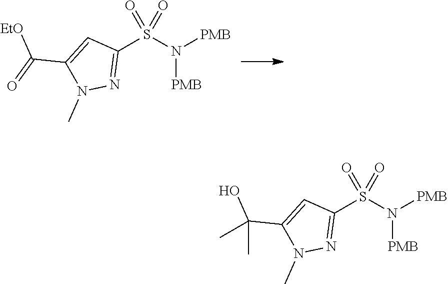

Ethyl 3-(N,N-bis(4-methoxybenzyl)sulfamoyl)-1-methyl-1H-pyrazole-5-carboxylate (1.4 g, 2.96 mmol) was dissolved in dry tetrahydrofuran (50 mL) and cooled to −78° C. in a dry ice/acetone bath. Methylmagnesium chloride (3 M in tetrahydrofuran, 5 ml, 15.00 mmol) was added slowly via syringe over the course of 15 minutes. The reaction mixture was allowed to reach room temperature and stirred overnight before being cooled in an ice bath and then quenched slowly with portions of aqueous ammonium chloride (20 mL). The mixture was extracted into ethyl acetate (3×50 mL) and the combined organic washings were washed with brine (10 to mL), dried (sodium sulfate), filtered and concentrated in vacua to afford a colourless oil. The crude product was purified by chromatography on silica (40 g column, 0-50% ethyl acetate/iso-hexane) to afford the title compound (1.11 g, 67%) as a thick colourless oil.

¹H NMR (DMSO-d₆) δ 7.09-7.03 (m, 4H), 6.85-6.80 (m, 4H), 6.41 (s, 1H), 4.21 (s, 4H), 4.04 (s, 3H), 3.72 (s, 6H) and 1.50 (s, 6H).

LCMS m/z 460 (M+H)⁺ (ES⁺); 458 (M−H)⁻ (ES⁻).

### Step C: N,N-Bis-(4-methoxybenzyl)-5-(2-methoxypropan-2-yl)-1-methyl-1H-pyrazole-3-sulfonamide

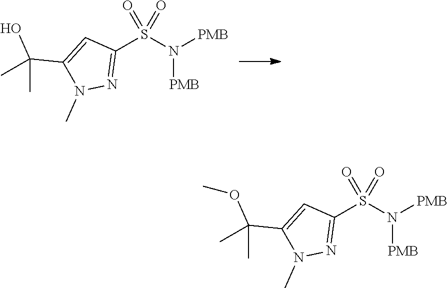

5-(2-Hydroxypropan-2-yl)-N,N-bis(4-methoxybenzyl)-1-methyl-rH-pyrazole-3-sulfonamide (2.5 g, 5.33 mmol) was dissolved in dry N,N-dimethylformamide (50 mL) under nitrogen atmosphere. After cooling in an ice bath, sodium hydride (60% in mineral oil, 0.25 g, 6.25 mmol) was added in a single portion and the cloudy yellow mixture was stirred for 30 minutes. Iodomethane (1.5 ml, 24.09 mmol) was added in a single portion and the mixture was stirred for a further 2 hours while warming to room temperature. The reaction mixture was quenched by slow addition of saturated aqueous ammonium chloride (10 mL) and then partitioned between ethyl acetate (100 mL) and water (50 mL). The aqueous phase was extracted with ethyl acetate (4×50 mL) and the combined organic portions were washed with brine (20 mL), dried (sodium sulfate), filtered and concentrated in vacua to give a yellow oil. The crude product was purified by chromatography on silica (40 g column, 0-100% ethyl acetate/iso-hexane) to afford, after drying in vacua, the title compound (2.41 g, 94%) as a colourless solid.

¹H NMR (DMSO-d₆) δ 7.10-7.04 (m, 4H), 6.85-6.80 (m, 4H), 6.48 (s, 1H), 4.23 (s, 4H), 3.97 (s, 3H), 3.72 (s, 6H), 2.97 (s, 3H) and 1.50 (s, 6H).

LCMS m/z 474 (M+H)⁺ (ES⁺); 472 (M−H)⁻ (ES⁻).

### Step D: 5-(2-Methoxypropan-2-yl)-1-methyl-1H-pyrazole-3-sulfonamide

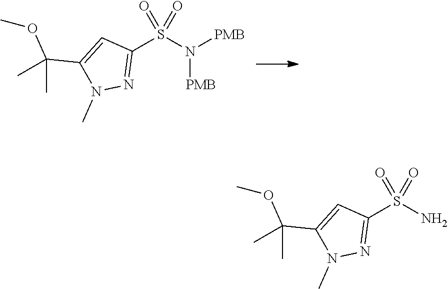

N,N-Bis-(4-methoxybenzyl)-5-(2-methoxypropan-2-yl)-1-methyl-1H-pyrazole-3-sulfonamide (2.4 g, 5.02 mmol) was dissolved in acetonitrile (40 mL). A solution of ceric ammonium nitrate (15 g, 27.4 mmol) in water (10 mL) was added in a single portion and the dark red reaction mixture was stirred at room temperature for 4 hours. Water (10 mL) and dichloromethane (250 mL) were added and the organic phase was separated, dried by passing through a hydrophobic frit and concentrated in vacuo to give an orange oil (˜2.5 g). The crude product was purified by chromatography on silica (40 g column, 0-20% methanol/dichloromethane) to afford an orange oil. Trituration of this material in tert-butylmethylether (10 mL) and iso-hexanes (5 mL) gave a tan precipitate which was further purified by chromatography on silica (24 g, 20-100% ethyl acetate in hexanes) to afford the title compound (383 mg, 31%) as a yellow solid.

¹H NMR (CDCl₃) δ 6.57 (s, 1H), 5.08 (s, 2H), 4.06 (s, 3H), 3.08 (s, 3H) and 1.57 (s, 6H).

### Step E: (4-(Dimethylamino)pyridin-1-ium-1-carbonyl)((5-(2-methoxypropan-2-yl)-1-methyl-1H-pyrazol-3-yl)sulfonyl)amide

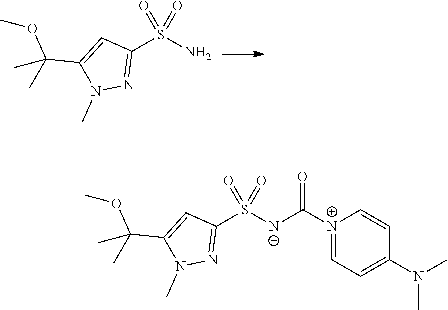

A solution of 5-(2-methoxypropan-2-yl)-1-methyl-1H-pyrazole-3-sulfonamide (160 mg, 0.686 mmol) in acetonitrile (0.8 mL) was treated with N,N-dimethylpyridin-4-amine (168 mg, 1.372 mmol) and the reaction mixture was stirred at room temperature until sulfonamide had dissolved. Diphenyl carbonate (162 mg, 0.754 mmol) was added and the reaction mixture was left for 16 hours at room temperature. The resulting precipitate was filtered, washed with methyl tert-butylether and dried to afford the title compound (46 mg, 18%) as a white solid which was used without further purification.

¹H NMR (CDCl₃) δ 9.03 (d, J=7.9 Hz, 2H), 6.77 (s, 1H), 6.74 (d, J=7.8 Hz, 2H), 4.04 (s, 3H), 3.34 (s, 6H), 3.08 (s, 3H), 1.59 (s, 6H).

### Intermediate P6: (4-(Dimethylamino)pyridin-1-ium-1-carbonyl)((1-isopropyl-1H-imidazol-4-yl)sulfonyl)amide

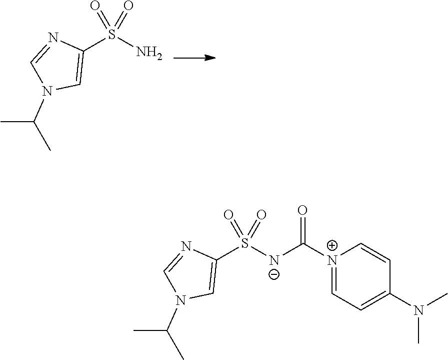 [SMILES: CC(C)n1cnc(S(N)(=O)=O)c1 ; CC(C)n1cnc(S(=O)(=O)[N-]C(=O)[n+]2ccc(N(C)C)cc2)c1]

A solution of 1-iso-propyl-1H-imidazole-4-sulfonamide (161 mg, 0.851 mmol) in acetonitrile (1 mL) was treated with N,N-dimethylpyridin-4-amine (208 mg, 1.702 mmol) and the reaction mixture was stirred at room temperature until sulfonamide had dissolved. Then diphenyl carbonate (200 mg, 0.936 mmol) was added and the reaction mixture was left for 16 hours at room temperature. The resulting precipitate was separated by filtration, washed with methyl tert-butylether and dried to afford the title compound (186 mg, 65%) as a white solid which was used without further purification.

### Intermediate P7: 5-((Dimethylamino)methyl)-1-ethyl-1H-pyrazole-3-sulfonamide

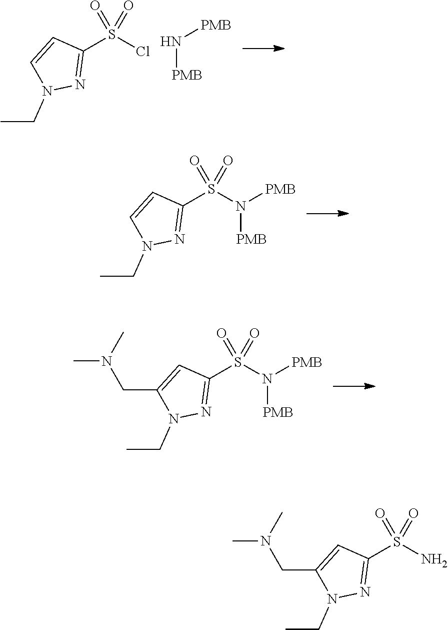

The title compound was prepared according to the procedure for 5-((dimethylamino) methyl)-1-methyl-1H-pyrazole-3-sulfonamide (Intermediate P1) (705 mg, 81%).

¹H NMR (DMSO-d6) δ 7.35 (s, 2H), 6.47 (s, 1H), 4.19 (q, J=7.2 Hz, 2H), 3.47 (s, 2H), 2.17 (s, 6H), 1.35 (t, J=7.2 Hz, 3H).

LCMS m/z 233.4 (M+H)⁺ (ES⁺).

### Intermediate P8: 5-(3-Methoxyoxetan-3-yl)-1-methyl-1H-pyrazole-3-sulfonamide

### Step A: 5-(3-Hydroxyoxetan-3-yl)-N,N-bis(4-methoxybenzyl)-1-methyl-1H-pyrazole-3-sulfonamide

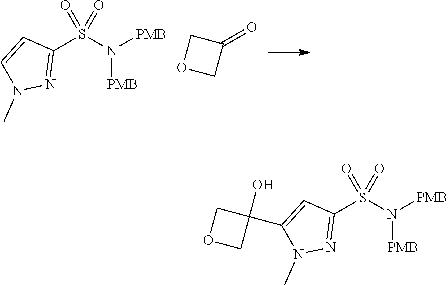

2.5 M n-Butyllithium in hexanes (2.0 mL, 5.00 mmol) was added dropwise to a stirred solution of N,N-bis(4-methoxybenzyl)-1-methyl-1H-pyrazole-3-sulfonamide (2.0 g, 4.98 mmol) in THF (35 mL) cooled to −78° C. and stirred for 1 hour. A solution of oxetan-3-one (0.292 mL, 4.98 mmol) in THF (16 mL) was then added, allowed to warm to room temperature with stirring for a further 1 hour. The reaction was quenched with saturated aq. NH₄Cl solution (20 mL) and extracted with EtOAc (3×50 mL). The combined extracts were washed with brine (20 mL), dried (MgSO₄), filtered and evaporated in vacuo to give an orange oil. The crude product was purified by chromatography on silica gel (80 g column, 0-75% EtOAc/isohexane) to afford the title compound (1.44 g, 61%) as a colourless solid.

¹H NMR (DMSO-d6) δ 7.10-7.00 (m, 4H), 6.90 (s, 1H), 6.85-6.78 (m, 4H), 6.75 (s, 1H), 4.89 (d, J=7.3 Hz, 2H), 4.76 (d, J=7.2 Hz, 2H), 4.23 (s, 4H), 3.81 (s, 3H), 3.71 (s, 6H).

LCMS m/z 496.1 (M+Na)⁺ (ES⁺).

### Step B: N,N-Bis(4-methoxybenzyl)-5-(3-methoxyoxetan-3-yl)-1-methyl-1H-pyrazole-3-sulfonamide

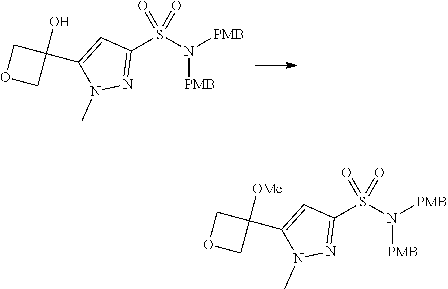

Sodium hydride (60% in mineral oil) (0.193 g, 4.81 mmol) was added portionwise to 5-(3-hydroxyoxetan-3-yl)-N,N-bis(4-methoxybenzyl)-1-methyl-1H-pyrazole-3-sulfonamide (2.00 g, 4.01 mmol) in dry DMF (20 mL) at 0° C. The reaction mixture was stirred for 30 minutes at 0° C., then 2M iodomethane in tert-butyl methyl ether (8.02 mL, 16.05 mmol) was added in a single portion and the mixture was stirred for a further 18 hours while warming to room temperature. The reaction mixture was quenched by slow addition of saturated aq. NH₄Cl (10 mL) and then partitioned between EtOAc (30 mL) and brine (100 mL). The aqueous layer was separated and the organic layer was washed with brine (100 mL). The organic layer was dried (MgSO₄), filtered and concentrated in vacua to give a pale yellow solid. The crude product was purified by chromatography on silica gel (24 g column, 0-70% EtOAc/isohexane) to afford the title compound (1.94 g 92%) as a colourless oil.

¹H NMR (DMSO-d6) δ 7.12-7.03 (m, 4H), 7.00 (s, 1H), 6.87-6.78 (m, 4H), 4.87 (d, J=7.7 Hz, 2H), 4.78 (d, J=7.7 Hz, 2H), 4.24 (s, 4H), 3.74 (s, 3H), 3.71 (s, 6H), 2.96 (s, 3H).

LCMS m/z 488.2 (M+H)⁺ (ES^{+).}

### Step C: 5-(3-Methoxyoxetan-3-yl)-1-methyl-1H-pyrazole-3-sulfonamide

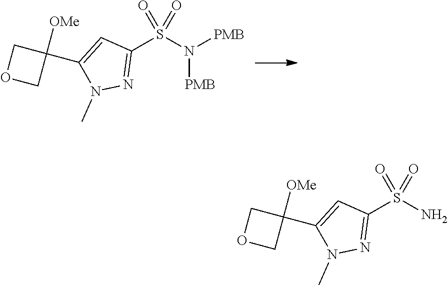

N,N-Bis(4-methoxybenzyl)-5-(3-methoxyoxetan-3-yl)-1-methyl-1H-pyrazole-3-sulfonamide (1.93 g, 3.60 mmol) was dissolved in acetonitrile (25 mL). A solution of ceric ammonium nitrate (9.87 g, 18.01 mmol) in water (16 mL) was added portionwise over 5 minutes. The orange mixture was stirred for 17 hours at room temperature, then concentrated to ˜20 mL and poured onto EtOAc (30 mL). The organic layer was separated and the aqueous layer was extracted with EtOAc (2×30 mL). The combined organic layers were dried (MgSO₄), filtered and concentrated to dryness to give an orange oil. The crude product was purified by chromatography on reversed phase flash column C18 (130 g column, 0-20% acetonitrile/10 mM ammonium bicarbonate, monitored at 215 nm), then purified further by chromatography on silica gel (40 g column, 0-10% methanol/dichloromethane) to afford the title compound (357 mg, 40%) as a tan solid.

¹H NMR (DMSO-d6) δ 7.46 (s, 2H), 6.92 (s, 1H), 4.94-4.82 (m, 2H), 4.83-4.70 (m, 2H), 3.73 (s, 3H), 2.99 (s, 3H).

LCMS m/z 248.3 (M+H)⁺ (ES⁺).

### Intermediate P9: 1-(2-(Dimethylamino)ethyl)-1H-pyrazole-3-sulfonamide

### Step A: Lithium 1-(tetrahydro-2H-pyran-2-yl)-1H-pyrazole-5-sulfinate

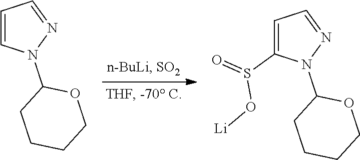

To a solution of 1-(tetrahydro-2H-pyran-2-yl)-1H-pyrazole (100 g, 657.06 mmol, 1 eq) in THF (1380 mL) was added n-BuLi (2.5 M, 276 mL, 1.05 eq) slowly keeping the temperature at −70° C. The reaction mixture was stirred for 1.5 hours, then SO₂ was bubbled into the mixture for 15 minutes. After the reaction temperature was heated to 25° C., a lot of solid was formed. The mixture was concentrated in vacua. The residue was triturated with tert-butyl methyl ether (400 mL) and the mixture was filtered. The filter cake was washed with tert-butyl methyl ether, n-hexane and dried to afford the title compound (142 g, crude) as a white solid.

¹H NMR (DMSO-d₆) δ 7.28 (d, 1H), 6.16 (d, 1H), 5.97 (dd, 1H), 3.92-3.87 (m, 1H), 3.61-3.53 (m, 1H), 2.25-2.18 (m, 1H), 1.98-1.93 (m, 1H), 1.78-1.74 (m, 1H) and 1.52-1.49 (m, 3H).

LCMS: m/z 215 (M-Li)⁻ (ES⁻)

### Step B: 1-(Tetrahydro-2H-pyran-2-yl)-1H-pyrazole-5-sulfonyl chloride

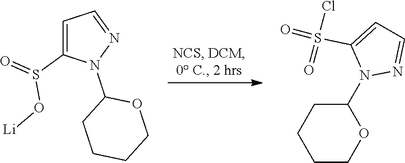

To a suspension of lithium 1-(tetrahydro-2H-pyran-2-yl)-1H-pyrazole-5-sulfinate (20 g, 90.01 mmol, 1 eq) in dichloromethane (250 mL) was added NCS (12.02 g, 90.01 mmol, 1 eq) cooled in an ice bath. The mixture was stirred at 0° C. for 2 hours. The solution was quenched with water (100 mL), then partitioned between dichloromethane (300 mL) and water (200 mL). The organic layer was washed with water (200 mL), dried over anhydrous magnesium sulfate, filtered and concentrated in vacuo to give the title compound (15.8 g, 63.02 mmol, 70%) as a yellow oil which was used directly in next step.

### Step C: N,N-Bis(4-methoxybenzyl)-1-(tetrahydro-2H-pyran-2-yl)-1H-pyrazole-5-sulfonamide

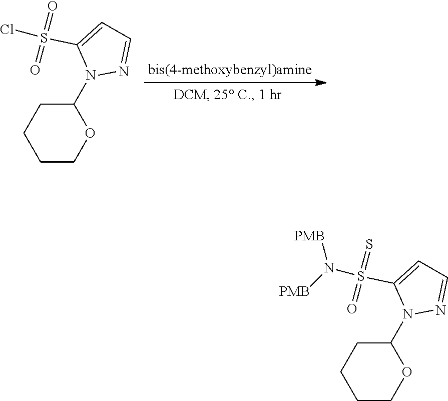

A solution of 1-(tetrahydro-2H-pyran-2-yl)-1H-pyrazole-5-sulfonyl chloride (15 g, 59.83 mmol, 1 eq) in dichloromethane (50 mL) was added to a mixture of bis(4-methoxybenzyl)amine (16.01 g, 62.23 mmol, 1.04 eq) and triethylamine (19.33 g, 190.99 mmol, 26.58 mL, 3.19 eq) in dichloromethane (300 mL) at 0° C. The reaction mixture was stirred at 0° C. for 1 hour and then quenched with water (250 mL). The organic layer was washed with water (250 mL), 1M HCl aqueous solution (2×250 mL), water (250 mL), dried over anhydrous MgSO₄, filtered, and concentrated in vacua to afford the title product (25.5 g, 49.75 mmol, 83% yield, 92% purity) as a brown oil.

LCMS: m/z 494 (M+Na)⁺ (ES⁺).

### Step D: N,N-Bis(4-methoxybenzyl)-1H-pyrazole-5-sulfonamide

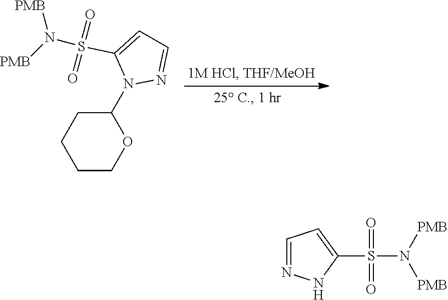

To a solution of N,N-bis(4-methoxybenzyl)-1-(tetrahydro-2H-pyran-2-yl)-1H-pyrazole-5-sulfonamide (25 g, 53.01 mmol, 1 eq) in THF (183 mL) and MeOH (37 mL) was added 1M HCl aqueous solution (18.29 mL, 0.34 eq) and the mixture was stirred at 25° C. for 1 hour. Then the solvent was evaporated and the residue was partitioned between dichloromethane (200 ml) and H₂O (100 mL). The organic layer was washed with brine (100 mL), dried over anhydrous MgSO₄, filtered and concentrated in vacua. The residue was triturated with tert-butyl methyl ether, filtered and dried to afford the title compound (12.2 g, 30.61 mmol, 58% yield, 97% purity) as a white solid.

¹H NMR (chloroform-d) δ 13.82-13.70 (br s, 1H), 7.92 (d, 1H), 7.07-7.01 (m, 4H), 6.78-6.75 (m, 4H), 6.61 (d, 1H), 4.34 (s, 4H) and 3.80 (s, 6H).

LCMS: m/z 410 (M+Na)⁺ (ES⁺).

### Step E: 1-(2-Hydroxyethyl)-N,N-bis(4-methoxybenzyl)-1H-pyrazole-3-sulfonamide

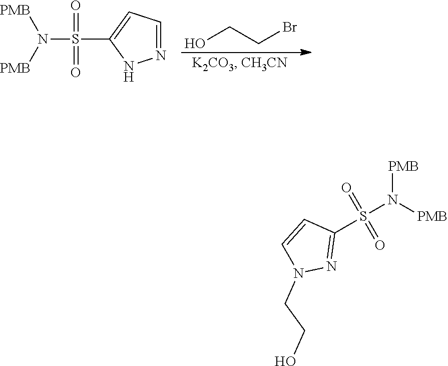

N,N-Bis(4-methoxybenzyl)-1H-pyrazole-5-sulfonamide (12 g, 30.97 mmol, 1 eq) and K₂CO₃ (8.39 g, 60.70 mmol, 1.96 eq) were suspended in acetonitrile (150 mL) under a nitrogen atmosphere. 2-Bromoethanol (5.03 g, 40.26 mmol, 2.86 mL, 1.3 eq) was added to this mixture and then the mixture was heated to 60° C. for 17 hours. To the reaction mixture was added water (500 mL) and dichloromethane (400 mL). The organic layer was separated and washed with brine (300 mL), dried over anhydrous Na₂SO₄, filtered and concentrated in vacua. The crude product was purified by chromatography on silica gel (petroleum ether:ethyl acetate=30:1 to 1:1) to give the title compound (8 g, 17.98 mmol, 58% yield, 97% purity) as a yellow oil.

¹H NMR (chloroform-d) δ 7.55 (d, 1H), 7.04-7.02 (m, 4H), 6.77-6.74 (d, 4H), 6.06 (d, 1H), 4.29 (s, 4H), 4.26-4.23 (t, 2H), 3.93-3.81 (m, 2H) and 3.69 (s, 6H).

LCMS: m/z 454 (M+Na)⁺ (ES⁺).

### Step F: 2-(3-(N,N-Bis(4-methoxybenzyl)sulfamoyl)-1H-pyrazol-1-yl)ethyl methanesulfonate

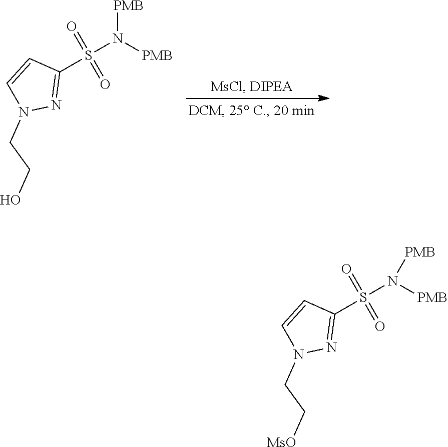

To a solution of 1-(2-hydroxyethyl)-N,N-bis(4-methoxybenzyl)-1H-pyrazole-3-sulfonamide (7 g, 16.22 mmol, 1 eq) and diisopropylethylamine (2.94 g, 22.71 mmol, 3.96 mL, 1.4 eq) in anhydrous dichloromethane (116 mL) was added methanesulfonyl chloride (2.23 g, 19.47 mmol, 1.51 mL, 1.2 eq) under nitrogen. The reaction mixture was stirred at 25° C. for 20 minutes. Then the mixture was quenched with saturated aqueous NaHCO₃ solution (50 mL) and water (30 mL). The organic layer was separated, dried over anhydrous Na₂SO₄, filtered and concentrated in vacua to give the title compound (8.3 g, crude) as a yellow oil which was used directly in the next step.

### Step G: 1-(2-(Dimethylamino)ethyl)-N,N-bis(4-methoxybenzyl)-1H-pyrazole-3-sulfonamide

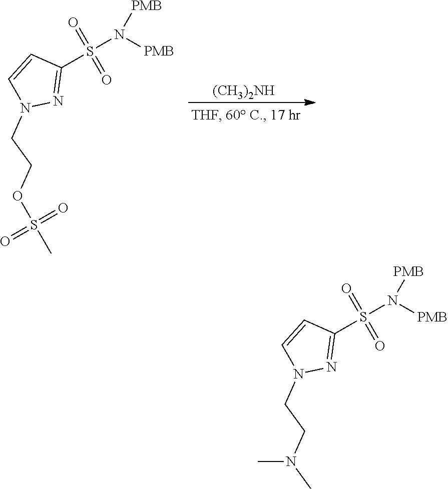

To a solution of dimethylamine in THF (2M, 243 mL, 29.95 eq) was added 2-(3-(N,N-bis(4-methoxybenzyl)sulfamoyl)-1H-pyrazol-1-yl)ethyl methanesulfonate (8.27 g, 16.23 mmol, 1 eq) and then the mixture was heated to 60° C. for 17 hours. The reaction mixture was concentrated in vacuo. The residue was added into EtOAc (150 mL) and the mixture was stirred and filtered. The organic phase was concentrated in vacuo and purified by column chromatography on silica gel (petroleum ether:ethyl acetate=10:1 to 0:1) to give the title compound (6.5 g, 13.47 mmol, 83% yield, 95% purity) as a yellow oil.

¹H NMR (chloroform-d) δ 7.55 (d, 1H), 7.09-7.06 (m, 4H), 6.81-6.78 (m, 4H), 6.65 (d, 1H), 4.31 (s, 4H), 4.31-4.27 (m, 2H), 3.80 (s, 6H), 2.77 (t, 2H) and 2.29 (m, 6H).

LCMS: m/z 459 (M+H)⁺ (ES⁺).

### Step H: 1-(2-(Dimethylamino)ethyl)-1H-pyrazole-3-sulfonamide

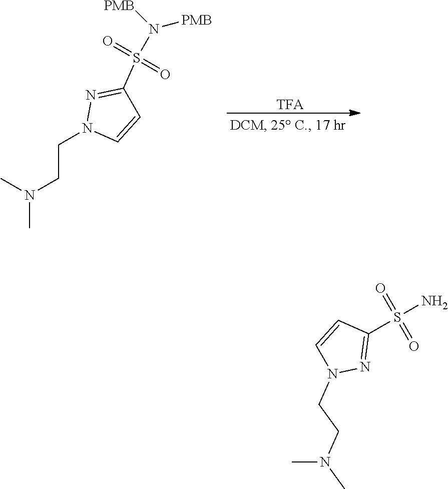

To a solution of 1-(2-(dimethylamino)ethyl)-N,N-bis(4-methoxybenzyl)-1H-pyrazole-3-sulfonamide (5.5 g, 11.99 mmol, 1 eq) in dichloromethane (10 to mL) was added trifluoroacetic acid (77.00 g, 675.32 mmol, 50 mL, 56.31 eq). The mixture was stirred at 25° C. for 17 hours. The reaction mixture was concentrated in vacua. The residue was dissolved in a mixture of dichloromethane (10 mL) and MeOH (200 mL). The resulting mixture was stirred and filtered. Basic resin was added to the solution until pH=8. Then the mixture was stirred at 25° C. for 30 minutes. The mixture was filtered and the organic phase was concentrated in vacua. The residue was recrystallized from dichloromethane (15 mL) to give the title compound (2.2 g, 10.08 mmol, 84% yield, 100% purity)

¹H NMR (DMSO-d₆) δ 7.86 (d, 1H), 7.37 (br s, 2H), 6.55 (d, 1H), 4.24 (t, 2H), 2.65 (t, 1H) and 2.16 (s, 6H).

LCMS: m/z 219 (M+H)⁺ (ES^{+).}

### Intermediate P10: 1-(Prop-2-yn-1-yl)piperidine-4-sulfonamide

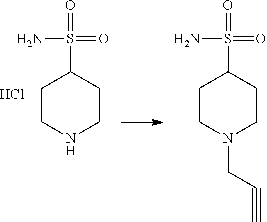

To a mixture of piperidine-4-sulfonamide hydrochloric acid (200 mg, 1.0 mmol, 1.0 equiv.), potassium carbonate (4.0 equiv., 4.0 mmol, 552 mg) and acetonitrile (10 mL) was added propargyl bromide (0.1 mL, 1.0 mmol, 1.0 equiv.). After stirring overnight at room temperature, the reaction mixture was concentrated in vacua and the crude material was suspended in methanol, coated on Agilent hydromatrix (a high purity, inert diatomaceous earth sorbent) and then submitted to normal phase flash chromatography using dichloromethane and a mixture of ammonia (3.5 M) in methanol to afford the title compound (115 mg, 56%).

¹H NMR (CDCl₃): δ 4.42 (br s, 1H), 3.38 (s, 2H), 3.05 (d, 2H), 2.95 (m, 1H), 2.12 (m, 4H) and 1.95 (m, 2H).

### Intermediate P11: 1-Ethylpiperidine-4-sulfonamide

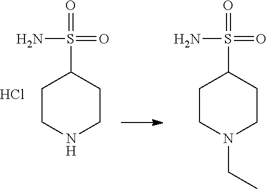

Prepared as described for 1-(prop-2-yn-1-yl)piperidine-4-sulfonamide (Intermediate P10) using ethyliodide instead of propargyl bromide. The crude product was coated on Agilent hydromatrix (a high purity, inert diatomaceous earth sorbent) and was submitted to normal phase flash chromatography using dichloromethane and a mixture of trimethylamine-methanol (ratio 1:1) as eluent to afford the title compound contaminated with triethylamine hydrochloride (50 mg, yield 26%). The crude product was used as such in preparing examples.

¹H NMR (CDCl₃): δ 5.05 (br s, 2H), 3.10 (m, 2H), 2.95 (m, 1H), 2.45 (m, 2H), 2.20 (d, 2H), 1.95 (m, 4H) and 1.08 (t, 3H).

### Intermediate P12: 1-Isopropyl-6-oxo-1,6-dihydropyridine-3-sulfonamide

### Step A: 6-Chloro-N,N-bis(4-methoxybenzyl)pyridine-3-sulfonamide

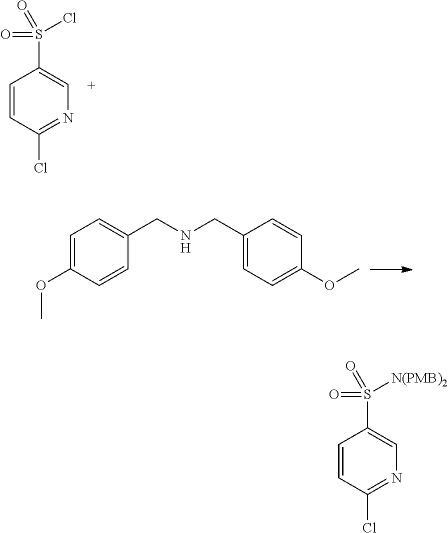

Bis(4-methoxybenzyl)amine (3.71 g, 14.4 mmol) was added to a solution of 2-chloropyridine-5-sulfonyl chloride (3.00 g, 13.7 mmol) and triethylamine (2.49 mL, 17.8 mmol) in DCM (50 mL) at 0° C. The reaction was stirred at 0° C. for 15 minutes and then allowed to warm up to room temperature and stirred for 20 hours. Then the reaction mixture was diluted with DCM (150 mL), washed with a saturated aqueous NH₄Cl solution (3×40 mL) and brine (40 mL), dried over MgSO₄, filtered, and concentrated in vacuo to give the crude product as a cream solid. The crude product was triturated with TBME (70 mL), filtered and rinsed with TBME (2×40 mL) to afford the title compound (4.97 g, 83%) as an off-white solid.

¹H NMR (DMSO-d6) δ 8.76 (dd, J=2.6, 0.7 Hz, 1H), 8.19 (dd, J=8.4, 2.6 Hz, 1H), 7.69 (dd, J=8.4, 0.7 Hz, 1H), 7.08-7.02 (m, 4H), 6.83-6.76 (m, 4H), 4.29 (s, 4H), 3.71 (s, 6H).

LCMS: m/z 433.3 (M+H)⁺ (ES⁺).

### Step B: 6-Hydroxy-N,N-bis(4-methoxybenzyl)pyridine-3-sulfonamide

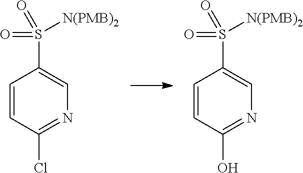

A suspension of 6-chloro-N,N-bis(4-methoxybenzyl)pyridine-3-sulfonamide (0.508 g, 1.17 mmol) in ethane-1,2-diol (10 mL) was treated with 2 M KOH (aq) (2.4 mL, 4.80 mmol). The resultant suspension was stirred at 140° C. for 18 hours. Then the reaction mixture was treated with further 2 M KOH (aq) (0.6 mL, 1.2 mmol, 1 eq) and heated at 140° C. for another 6 hours, and further 2 M KOH (aq) (0.6 mL, 1.2 mmol, 1 eq) and heated at 140° C. for another 18 hours. Then the reaction mixture was diluted with water (40 mL) and DCM (30 mL). Brine (5 mL) was added and the organic layer was collected. The aqueous phase was extracted with DCM (5×30 mL). The combined organic extracts were washed with water (10 mL), dried over MgSO₄, filtered and concentrated in vacuo. The residue was dried under reduced pressure at 50° C. overnight to afford the title compound (542 mg, 100%).

¹H NMR (DMSO-d6) δ 12.17 (s, 1H), 7.86 (d, J=2.8 Hz, 1H), 7.63 (dd, J=9.6, 2.9 Hz, 1H), 7.11-7.02 (m, 4H), 6.87-6.79 (m, 4H), 6.37 (d, J=9.6 Hz, 1H), 4.21 (s, 4H), 3.72 (s, 6H).

LCMS: m/z 415.4 (M+H)⁺ (ES⁺), 413.4 (M−H) (ES⁻).

### Step C: 1-Isopropyl-N,N-bis(4-methoxybenzyl)-6-oxo-1,6-dihydropyridine-3-sulfonamide and 6-isopropoxy-N,N-bis(4-methoxybenzyl)pyridine-3-sulfonamide

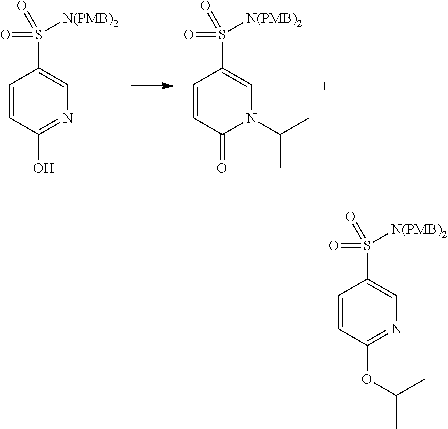

Sodium hydride (60 wt % dispersion in mineral oil) (36 mg, 0.91 mmol) was added to a mixture of 6-hydroxy-N,N-bis(4-methoxybenzyl)pyridine-3-sulfonamide (0.40 g, 0.869 mmol) and lithium bromide (0.154 g, 1.737 mmol) in DME:DMF (5 mL, 4:1) at 0° C. The mixture was stirred at 0° C. for 10 minutes and then at room temperature for a further 10 minutes. Then 2-iodopropane (0.10 mL, 1.04 mmol) was added and the mixture was stirred at room temperature for 46 hours. The reaction mixture was heated to 65° C. for 17 hours, cooled to room temperature and quenched with saturated aqueous NH₄Cl (5 mL) and diluted with EtOAc (100 mL). The organic layer was washed with water (15 mL) and brine (3×15 mL), dried over MgSO₄, filtered, and concentrated in vacuo. The crude product was purified by chromatography on silica gel (24 g column, 0-100% EtOAc/isohexane) to afford 1-isopropyl-N,N-bis(4-methoxybenzyl)-6-oxo-1,6-dihydropyridine-3-sulfonamide (0.28 g, 70%) as a white solid and 6-isopropoxy-N,N-bis(4-methoxybenzyl)pyridine-3-sulfonamide (0.11 g, 27%).

### 1-Isopropyl-N,N-bis(4-methoxybenzyl)-6-oxo-1,6-dihydropyridine-3-sulfonamide

¹H NMR (CDCl₃) δ 7.91 (d, J=2.7 Hz, 1H), 7.41 (dd, J=9.6, 2.6 Hz, 1H), 7.09-7.04 (m, 4H), 6.84-6.79 (m, 4H), 6.54 (dd, J=9.6, 0.5 Hz, 1H), 5.17 (sept, J=6.8 Hz, 1H), 4.26 (s, 4H), 3.79 (s, 6H), 1.34 (d, J=6.8 Hz, 6H).

LCMS: m/z 457.4 (M+H)⁺ (ES⁺).

### 6-Isopropoxy-N,N-bis(4-methoxybenzyl)pyridine-3-sulfonamide

¹H NMR (CDCl₃) δ 8.60-8.55 (m, 1H), 7.84-7.79 (m, 1H), 7.06-6.99 (m, 4H), 6.81-6.75 (m, 4H), 6.72-6.67 (m, 1H), 5.43-5.33 (m, 1H), 4.26 (s, 4H), 3.78 (s, 6H), 1.37 (d, J=6.2 Hz, 6H).

LCMS: m/z 457.4 (M+H)⁺ (ES⁺).

### Step D: 1-Isopropyl-6-oxo-1,6-dihydropyridine-3-sulfonamide

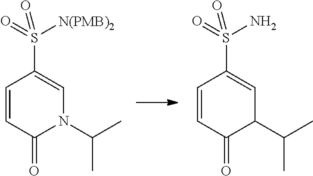

TFA (0.43 ml, 5.64 mmol) was added to a solution of 1-isopropyl-N,N-bis(4-methoxybenzyl)-6-oxo-1,6-dihydropyridine-3-sulfonamide (0.26 g, 0.564 mmol) in DCM (3 mL) at room temperature and the mixture was stirred for 66 hours. Then the reaction was concentrated in vacuo and the residue was redissolved in DCM (5 mL). The product was purified by chromatography on silica gel (12 g column, 0-10% MeOH/DCM) to afford the title compound (60 mg, 49%) as a white solid.

LCMS: m/z 217.3 (M+H)⁺ (ES⁺).

### Intermediate P13: 4-Isopropyl-5-oxo-4,5-dihydropyrazine-2-sulfonamide

### Step A: 2-(Benzylthio)-5-chloropyrazine

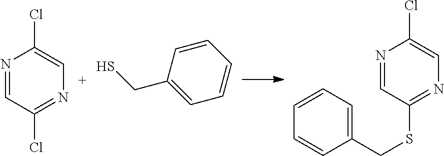 [SMILES: Clc1cnc(Cl)cn1 ; SCc1ccccc1 ; Clc1cnc(SCc2ccccc2)cn1]

To a solution of NaH (0.755 g, 18.88 mmol) in THF (55 mL) was added benzyl mercaptan (1.5 mL, 12.68 mmol) at 0° C. The reaction mixture was diluted with THF (20 mL) and stirred at 0° C. for 10 minutes. Then a solution of 2,5-dichloropyrazine (1.370 mL, 13.42 mmol) in THF (10 mL) was added dropwise. The reaction mixture was stirred at 0° C. for 1 hour, then warmed to room temperature and stirred for 16 hours. The reaction mixture was cooled to 0° C., MeOH (1 mL) was added carefully and stirred for 5 minutes. Water (20 mL), then DCM (150 mL) was added and the biphasic mixture was passed through a phase separator. The organic phase was concentrated in vacua. The crude product was purified by chromatography on silica gel (40 g column, 0-3% EtOAc/isohexane) to afford the title compound (2.373 g, 72%) as a clear yellow oil.

¹H NMR (DMSO-d6) δ 8.68 (d, J=1.5 Hz, 1H), 8.49 (d, J=1.5 Hz, 1H), 7.43-7.39 (m, 2H), 7.34-7.29 (m, 2H), 7.28-7.23 (m, 1H), 4.46 (s, 2H).

### Step B: 5-Chloro-N,N-bis(4-methoxybenzyl)pyrazine-2-sulfonamide

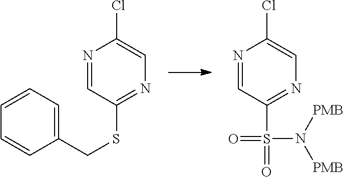

A solution of 2-(benzylthio)-5-chloropyrazine (0.916 g, 3.87 mmol) in DCM (15 mL, 233 mmol) was treated with water (1.5 mL) and the resultant suspension was cooled to between −5 and 0° C. Sulfuryl chloride (2.2 mL, 26.2 mmol) was added and the reaction mixture was stirred for 2 hours maintaining the temperature between −5 and 0° C. A slurry of ice/water (10 mL) was added and the organic phase was collected. The aqueous phase was extracted with DCM (2×10 mL) and the combined organic extracts were dried (MgSO₄) and concentrated in vacuo to afford crude intermediate 5-chloropyrazine-2-sulfonyl chloride as a pale yellow liquid (1.198 g).

A suspension of bis(4-methoxybenzyl)amine hydrochloride (1.198 g, 4.08 mmol) and TEA (1.2 mL, 8.61 mmol) in DCM (15 mL) at 0° C. was treated with a solution of 5-chloropyrazine-2-sulfonyl chloride (0.824 g, 3.87 mmol) in DCM (5 mL) dropwise. The resultant solution was stirred at 0° C. for 15 minutes and then allowed to warm to room temperature for 16 hours. A saturated aqueous NH₄Cl solution (10 mL) was added and the organic phase was collected. The aqueous phase was extracted with DCM (2×10 mL) and the combined organic extracts were dried (MgSO₄) and concentrated in vacua. The crude product was purified by chromatography on silica gel (24 g column, 0-30% EtOAc/isohexane) to afford the title compound (1.312 g, 77%) as a white solid.

¹H NMR (CDCl₃) δ 8.78 (d, J=1.4 Hz, 1H), 8.46 (d, J=1.4 Hz, 1H), 7.11-7.07 (m, 4H), 6.79-6.75 (m, 4H), 4.43 (s, 4H), 3.79 (s, 6H).

### Step C: N,N-Bis(4-methoxybenzyl)-5-oxo-4,5-dihydropyrazine-2-sulfonamide

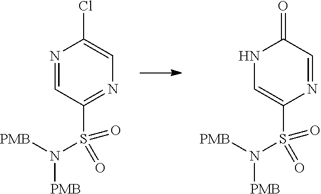

A suspension of 5-chloro-N,N-bis(4-methoxybenzyl)pyrazine-2-sulfonamide (1.31 g, 2.99 mmol) in glycol (15 mL) was treated with 2 M KOH (aq) (7.5 mL, 15 mmol). The resultant suspension was stirred at 140° C. for 18 hours. Then the reaction mixture was allowed to cool to room temperature, diluted with water (100 mL) and neutralised with saturated aqueous NH₄Cl solution (30 mL). The white precipitate was collected by filtration, washed with water and dried at 60° C. under vacuum to afford the title compound (1.094 g, 79%) as a pale yellow solid.

¹H NMR (DMSO-d6) δ 7.94 (d, J=1.2 Hz, 1H), 7.89 (br s, 1H), 7.10-7.06 (m, 4H), 6.84-6.79 (m, 4H), 4.28 (s, 4H), 3.71 (s, 6H). One exchangeable proton not observed.

LCMS: m/z 438.2 (M+Na)⁺ (ES⁺); 414.2 (M−H)⁻ (ES⁻).

### Step D: 4-Isopropyl-N,N-bis(4-methoxybenzyl)-5-oxo-4,5-dihydropyrazine-2-sulfonamide

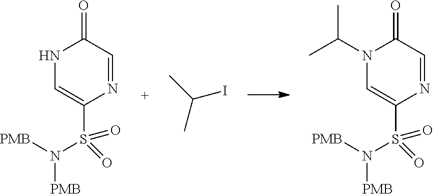

A suspension of N,N-bis(4-methoxybenzyl)-5-oxo-4,5-dihydropyrazine-2-sulfonamide (0.503 g, 1.090 mmol) and lithium bromide (0.192 g, 2.167 mmol) in DME:DMF (6 mL, 4:1) at 0° C. was treated with NaH (0.053 g, 1.325 mmol). The resultant suspension was stirred at 0° C. for 10 minutes, treated with 2-iodopropane (0.218 ml, 2.136 mmol) and then stirred at 65° C. for 64 hours. A saturated aqueous NH₄Cl solution (6 mL) and EtOAc (10 mL) were added and the organic layer was collected. The aqueous layer was extracted with EtOAc (2×10 mL) and the combined organic extracts were washed with water (10 mL) and brine (2×10 mL), dried (MgSO₄) and concentrated in vacua. The crude product was purified by chromatography on silica gel (12 g column, 0-100% EtOAc/isohexane) to afford the title compound (0.293 g, 53%) as a clear yellow oil.

¹H NMR (DMSO-d6) δ 8.07 (d, J=1.0 Hz, 1H), 7.96 (d, J=0.9 Hz, 1H), 7.13-7.09 (m, 4H), 6.83-6.79 (m, 4H), 4.78 (sept, J=6.5 Hz, 1H), 4.33 (s, 4H), 3.71 (s, 6H), 1.34 (d, J=6.8 Hz, 6H).

LCMS: m/z 480.3 (100, [M+Na]+), 458.5 (9, [M+H]⁺) (ES⁺).

### Step E: 4-Isopropyl-5-oxo-4,5-dihydropyrazine-2-sulfonamide

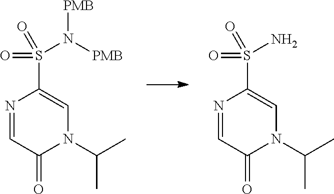

A solution of 4-isopropyl-N,N-bis(4-methoxybenzyl)-5-oxo-4,5-dihydropyrazine-2-sulfonamide (0.287 g, 0.565 mmol) in DCM (1 mL) was treated with TFA (1 mL, 12.98 mmol) at room temperature. The resultant solution was stirred for 28 hours. Then the reaction mixture was concentrated in vacuo and the crude product was purified by chromatography on silica gel (4 g column, 0-10% MeOH/DCM) to afford the title compound (0.116 g, 94%) as a white solid.

¹H NMR (DMSO-d6) δ 8.14 (d, J=1.0 Hz, 1H), 8.08 (d, J=1.0 Hz, 1H), 7.40 (s, 2H), 4.88 (sept, J=6.7 Hz, 1H), 1.36 (d, J=6.8 Hz, 6H).

LCMS: 216.1 (M−H)⁻ (ES⁻).

### Intermediate P14: 1-Isopropylazetidine-3-sulfonamide

### Step A: tert-Butyl 3-hydroxyazetidine-1-carboxylate

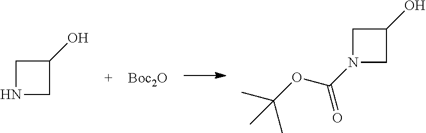

To a solution of azetidin-3-ol hydrochloride (45 g, 410.75 mmol, 1 eq) in MeOH (1.2 L) was added TEA (83.13 g, 821.51 mmol, 2 eq) and di-tert-butyl dicarbonate (89.65 g, 410.75 mmol, 1 eq). The mixture was stirred at 25° C. for 16 hours. Then the reaction mixture was concentrated in vacuo. The residue was re-dissolved in EtOAc (1 L). The mixture was washed with H₂O (3×500 mL) and brine (3×500 mL), dried over anhydrous Na₂SO₄, filtered and concentrated in vacua to give the title compound (65 g, 91%) as a yellow oil, which was used directly in the next step.

¹H NMR (CDCl₃) δ 4.59 (s, 1H), 4.19-4.12 (m, 2H), 3.4-3.79 (m, 2H), 1.45 (s, 9H).

### Step B: tert-Butyl 3-((methylsulfonyl)oxy)azetidine-1-carboxylate

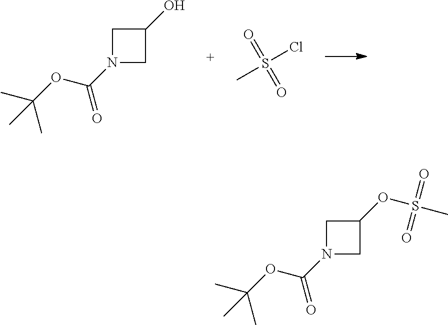

To a solution of tert-butyl 3-hydroxyazetidine-1-carboxylate (65 g, 375.27 mmol, 1 eq) and TEA (113.92 g, 3 eq) in THF (650 mL) was added methanesulfonyl chloride (51.58 g, 450.32 mmol, 1.2 eq) at 0° C. Then the mixture was stirred at 25° C. for 12 hours. The reaction mixture was diluted with EtOAc (2 L), washed with water (3×1.5 L) and brine (3×1.5 L), dried over anhydrous Na₂SO₄, filtered and concentrated in vacuo to give the title compound (90 g, 95%) as a yellow oil, which was used directly in the next step.

¹H NMR (CDCl₃) δ 5.25-5.20 (m, 1H), 4.32-4.27 (m, 2H), 4.14-4.10 (m, 2H), 3.08 (s, 3H) and 1.46 (s, 9H).

### Step C: tert-Butyl 3-(acetylthio)azetidine-1-carboxylate

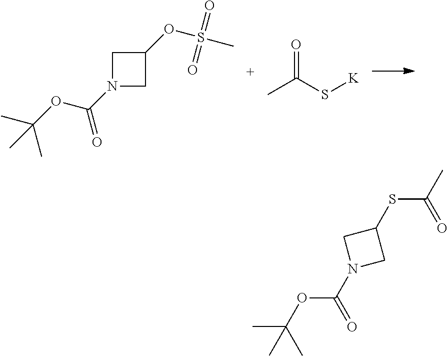

To a solution of tert-butyl 3-((methylsulfonyl)oxy)azetidine-1-carboxylate (90 g, 358.14 mmol, 1 eq) in DMF (1.5 L) was added potassium ethanethioate (49.08 g, 429.77 mmol, 1.2 eq). The mixture was stirred at 80° C. for 12 hours. Then the reaction mixture was diluted with EtOAc (3 L), washed with saturated aqueous NH₄Cl solution (3×2 L) and brine (3×2 L), dried over anhydrous Na₂SO₄, filtered and concentrated in vacua. The residue was purified by silica gel column chromatography (SiO₂, petroleum ether:ethyl acetate, 100:1 to 20:1) to give the title compound (54 g, 65%) as a yellow oil.

¹H NMR (CDCl₃) δ 4.37 (t, 2H), 4.17-4.14 (m, 1H), 3.82 (dd, 2H), 2.34 (s, 3H) and 1.44 (s, 9H).

### Step D: tert-Butyl 3-(chlorosulfonyl)azetidine-1-carboxylate

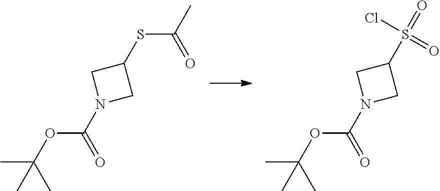

To a solution of tert-butyl 3-(acetylthio)azetidine-1-carboxylate (5 g, 21.62 mmol, 1 eq) in AcOH (200 mL) and H₂O (20 mL) was added NCS (8.66 g, 64.85 mmol, 3 eq). The reaction mixture was stirred at 25° C. for 1 hour. Then the reaction mixture was diluted with DCM (300 mL), washed with water (3×300 mL) and brine (3×300 mL), dried over anhydrous Na₂SO₄ and filtered. The solution was used directly in the next step.

### Step E: tert-Butyl 3-sulfamoylazetidine-1-carboxylate

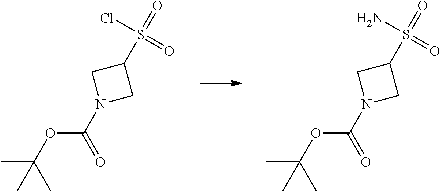

Through a solution of tert-butyl 3-(chlorosulfonyl)azetidine-1-carboxylate (55.28 g, crude) in DCM (1.5 L) was bubbled NH₃ for 30 minutes at 0° C. Then the reaction mixture was filtered and the filtrate was concentrated in vacuo. The residue was triturated with a mixture of petroleum ether and EtOAc (21 mL, 20:1) to give the title compound (27 g, 53%) as a white solid.

¹H NMR (DMSO-d₆) δ 7.16 (br s, 2H), 4.18-4.03 (m, 2H), 4.03-3.90 (m, 3H) and 1.38 (s, 9H).

### Step F: tert-Butyl 3-(N,N-bis(4-methoxybenzyl)sulfamoyl)azetidine-1-carboxylate

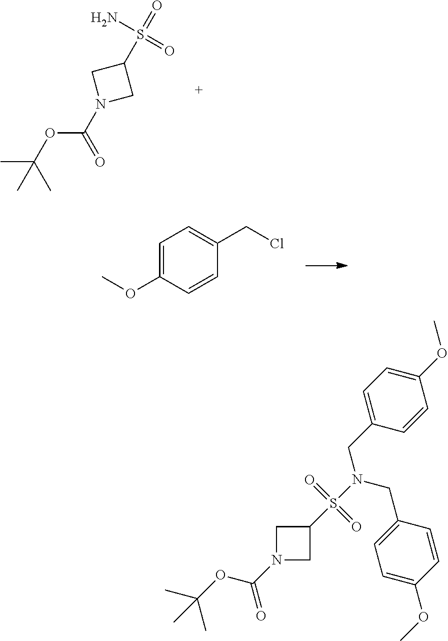

To a solution of tert-butyl 3-sulfamoylazetidine-1-carboxylate (1 g, 4.23 mmol, 1 eq) in DMF (10 mL) was added NaH (507 mg, 12.69 mmol, 60 wt % in mineral oil, 3 eq) at 0° C. The mixture was stirred at 0° C. for 30 minutes. Then 1-(chloromethyl)-4-methoxybenzene (1.99 g, 12.69 mmol, 3 eq) was added. The mixture was stirred at 25° C. for 14 hours. Then the reaction mixture was diluted with EtOAc (50 mL), washed with a saturated aqueous NH₄Cl solution (3×30 mL) and brine (3×30 mL), dried over anhydrous Na₂SO₄, filtered and concentrated in vacua. The residue was triturated with MeOH (10 mL) to give the title compound (1 g, 50%) as a white solid.

¹H NMR (CDCl₃) δ 7.17 (d, 4H), 6.91-6.88 (m, 4H), 4.30 (s, 4H), 4.22 (dd, 2H), 4.01 (t, 2H), 3.83 (s, 6H), 3.75-3.62 (m, 1H) and 1.44 (s, 9H).

LCMS: m/z 499.2 (M+Na)⁺ (ES⁺).

### Step G: N,N-Bis(4-methoxybenzyl)azetidine-3-sulfonamide

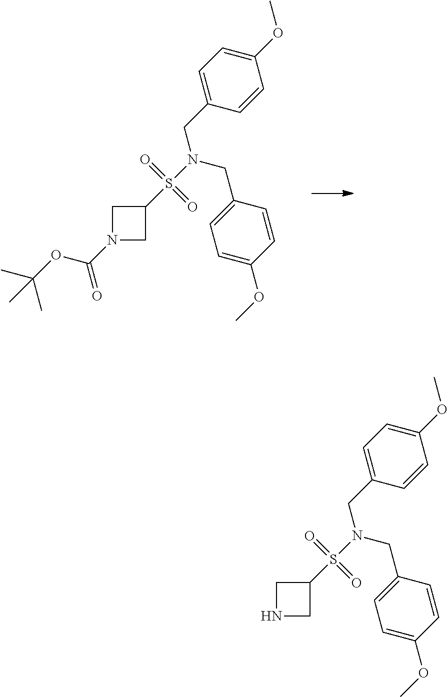 [SMILES: COc1ccc(CN(Cc2ccc(OC)cc2)S(=O)(=O)C2CN(C(=O)OC(C)(C)C)C2)cc1 ; COc1ccc(CN(Cc2ccc(OC)cc2)S(=O)(=O)C2CNC2)cc1]

To a solution of tert-butyl 3-(N,N-bis(4-methoxybenzyl)sulfamoyl)azetidine-1-carboxylate (7 g, 14.69 mmol, 1 eq) and 2,6-lutidine (4.72 g, 44.06 mmol, 3 eq) in DCM (80 mL) was added trimethylsilyl trifluoromethanesulfonate (9.79 g, 44.06 mmol, 3 eq) at 0° C. Then the reaction mixture was stirred at 0° C. for 1 hour. The reaction mixture was quenched with a saturated aqueous NH₄Cl solution (20 mL) and extracted with DCM (3×50 mL). The combined organic layers were dried over anhydrous Na₂SO₄, filtered and concentrated in vacua. The residue was triturated with a mixture of petroleum ether and ethyl acetate (40 mL, 1:1) to give the title compound (4 g, 72%) as a white solid.

¹H NMR (CD₃OD) δ 7.21 (d, 4H), 6.94-6.85 (m, 4H), 4.35 (s, 4H), 4.28-4.11 (m, 5H) and 3.81 (s, 6H).

LCMS: m/z 377.2 (M+H)⁺ (ES⁺).

### Step H: 1-Isopropyl-N,N-bis(4-methoxybenzyl)azetidine-3-sulfonamide

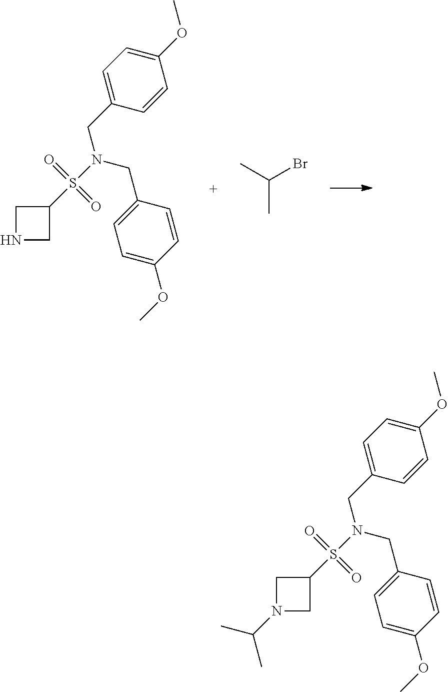

To a solution of N,N-bis(4-methoxybenzyl)azetidine-3-sulfonamide (2.5 g, 6.64 mmol, 1 eq) and K₂CO₃ (1.38 g, 9.96 mmol, 1.5 eq) in MeCN (5 mL) was added 2-bromopropane (1.63 g, 13.28 mmol, 2 eq). The mixture was stirred at 70° C. for 12 hours. Then H₂O (10 mL) was added and the reaction mixture was extracted with EtOAc (3×30 mL). The combined organic layers were dried over anhydrous Na₂SO₄, filtered and concentrated in vacua to give the title compound (2.5 g, 90%).

¹H NMR (CDCl₃) δ 7.12-7.07 (m, 4H), 6.83-6.76 (m, 4H), 4.16 (s, 4H), 3.74 (s, 6H), 3.68-3.64 (m, 1H), 3.43 (t, 2H), 3.28 (t, 2H), 2.38-2.29 (m, 1H) and 0.82 (d, 6H).

LCMS: m/z 419.2 (M+H)⁺ (ES⁺).

### Step I: 1-Isopropylazetidine-3-sulfonamide

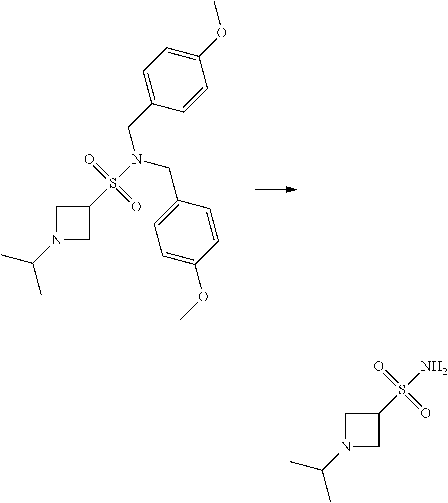 [SMILES: COc1ccc(CN(Cc2ccc(OC)cc2)S(=O)(=O)C2CN(C(C)C)C2)cc1 ; CC(C)N1CC(S(N)(=O)=O)C1]

A solution of 1-isopropyl-N,N-bis(4-methoxybenzyl)azetidine-3-sulfonamide (1 g, 2.39 mmol, 1 eq) in TFA (7.70 g, 67.53 mmol, 28.27 eq) was stirred at 25° C. for 12 hours. Then the reaction mixture was concentrated in vacua. The residue was treated with MeOH (10 mL), filtered and the filtrate was adjusted with NH₃.H₂O (30% of NH₃.H₂O in water) to pH=8-9. The resulting mixture was concentrated in vacuo. The residue was purified by reversed phase flash chromatography (water (0.1% of NH₃.H₂O)-MeCN) to give the title compound (220 mg, 52%) as a white solid.

¹H NMR (CD₃OD) δ 4.05-3.98 (m, 1H), 3.67 (t, 2H), 3.46 (t, 2H), 2.59-2.48 (m, 1H) and 0.97 (d, 6H). Two exchangeable protons not observed.

LCMS: m/z 179.1 (M+H)⁺ (ES⁺).

### Intermediate P15: 1-Cyclobutylazetidine-3-sulfonamide

### Step A: Azetidine-3-sulfonamide

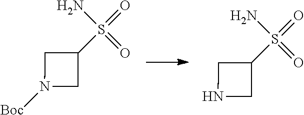 [SMILES: CC(C)(C)OC(=O)N1CC(S(N)(=O)=O)C1 ; NS(=O)(=O)C1CNC1]

To a solution of tert-butyl 3-sulfamoylazetidine-1-carboxylate (3 g, 12.70 mmol, 1 eq, obtained according to Step E of the synthesis of intermediate P14) in DCM (10 mL) was added HCl/EtOAc (12.70 mmol, 20 mL, 1 eq). The mixture was stirred at 25° C. for 1 hour. Then the reaction mixture was concentrated in vacuo. The residue was purified by reversed phase flash chromatography (water (0.05% of NH₃.H₂O)-MeCN) to give the title compound (0.8 g, 46%) as a white solid.

¹H NMR (DMSO-d₆) δ 6.92 (s, 1H), 4.23-4.19 (m, 2H) and 3.77-3.70 (m, 3H). Two exchangeable protons not observed.

LCMS: m/z 137.1 (M+H)⁺ (ES⁺).

### Step B: 1-Cyclobutylazetidine-3-sulfonamide

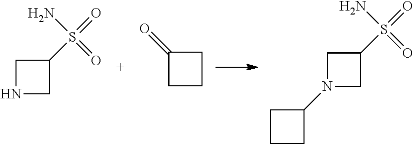 [SMILES: NS(=O)(=O)C1CNC1 ; O=C1CCC1 ; NS(=O)(=O)C1CN(C2CCC2)C1]

To a solution of azetidine-3-sulfonamide (50 mg, 367.18 μmol, 1 eq) in MeOH (1 mL) was added cyclobutanone (31 mg, 440.62 μmol, 1.2 eq) and NaBH(OAc)₃ (97 mg, 458.98 μmol, 1.25 eq). The reaction mixture was stirred at 20° C. for 2 hours. Then the reaction mixture was concentrated in vacuo. The residue was purified by reversed phase flash chromatography (water (0.05% of NH₃.H₂O)-MeCN) to give the title compound (12.25 mg, 18%) as a white solid.

¹H NMR (DMSO-d₆) δ 6.92 (s, 2H), 3.88-3.85 (m, 1H), 3.41-3.33 (m, 2H), 3.32-3.29 (m, 2H), 3.12-3.09 (m, 1H), 1.89-1.86 (m, 2H) and 1.77-1.60 (m, 4H).

LCMS: m/z 191.1 (M+H)⁺ (ES⁺).

### Intermediate P16: 1-Ethylazetidine-3-sulfonamide

### Step A: 1-Ethyl-N,N-bis(4-methoxybenzyl)azetidine-3-sulfonamide

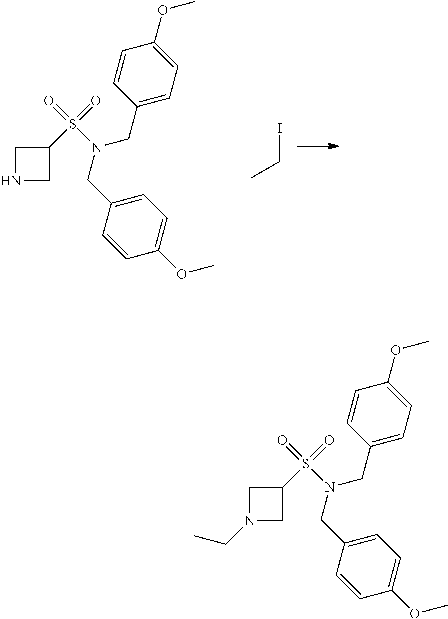

To a solution of N,N-bis(4-methoxybenzyl)azetidine-3-sulfonamide (1 g, 2.66 mmol, 1 eq, obtained according to Step G of the synthesis of intermediate P14) and K₂CO₃ (367 mg, 2.66 mmol, 1 eq) in MeCN (2 mL) was added iodoethane (414 mg, 2.66 mmol, 1 eq). The mixture was stirred at 70° C. for 1 hour. Then the reaction mixture was quenched with water (30 mL) and extracted with EtOAc (3×50 mL). The combined organic layers were dried over anhydrous Na₂SO₄, filtered and concentrated in vacua. The residue was purified by reversed phase flash chromatography (water (0.1% of NH₃.H₂O)-MeCN) to give the title compound (0.7 g, 22% yield, 100% purity on LCMS) as a white solid.

¹H NMR (CD₃OD) δ 7.20 (d, 4H), 6.90 (d, 4H), 4.28 (s, 4H), 4.00-3.93 (m, 1H), 3.81 (s, 6H), 3.51 (t, 2H), 3.40 (t, 2H), 2.53 (q, 2H) and 0.96 (t, 3H).

LCMS: m/z 405.2 (M+H)⁺ (ES⁺).

### Step B: 1-Ethylazetidine-3-sulfonamide

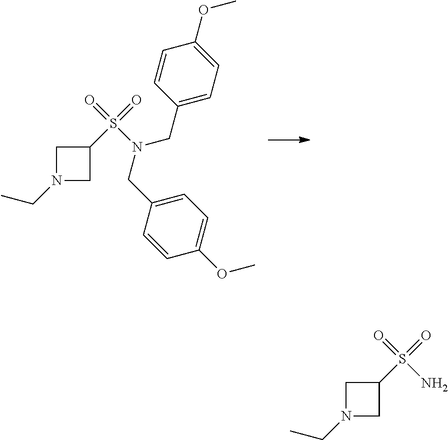

A solution of 1-ethyl-N,N-bis(4-methoxybenzyl)azetidine-3-sulfonamide (800 mg, 1.98 mmol, 1 eq) in TFA (82.13 g, 720.32 mmol, 364 eq) was stirred at 50° C. for 1 hour. Then the reaction mixture was concentrated in vacuo. The residue was purified by reversed phase flash chromatography (water (0.1% of NH₃.H₂O)-MeCN) to give the title compound (160 mg, 47% yield, 95% purity on LCMS) as a white solid.

¹H NMR (DMSO-d₆) δ 6.94 (s, 2H), 3.95-3.86 (m, 1H), 3.47 (t, 2H), 3.31-3.25 (m, 2H), 2.43 (q, 2H) and 0.86 (t, 3H).

LCMS: m/z 165.1 (M+H)⁺ (ES⁺).

### Intermediate P17: 1-(Pyridin-3-ylmethyl)azetidine-3-sulfonamide

### Step A: N,N-Bis(4-methoxybenzyl)-1-(pyridin-3-ylmethyl)azetidine-3-sulfonamide

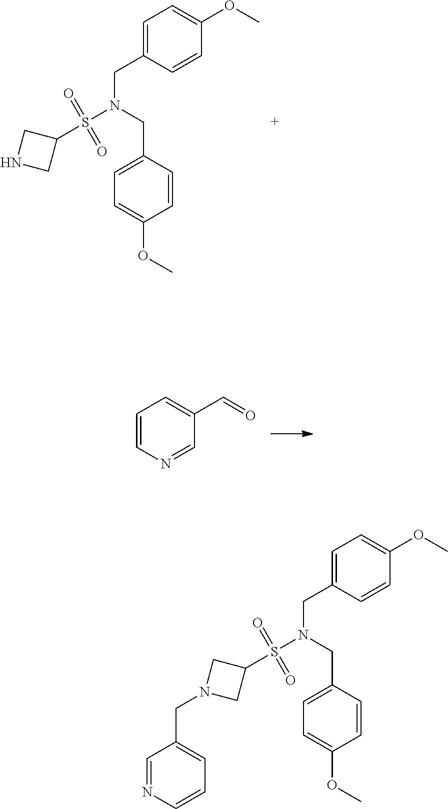 [SMILES: COc1ccc(CN(Cc2ccc(OC)cc2)S(=O)(=O)C2CNC2)cc1 ; O=Cc1cccnc1 ; COc1ccc(CN(Cc2ccc(OC)cc2)S(=O)(=O)C2CN(Cc3cccnc3)C2)cc1]

To a solution of N,N-bis(4-methoxybenzyl)azetidine-3-sulfonamide (1 g, 2.66 mmol, 1 eq, obtained according to Step G of the synthesis of intermediate P14) in MeCN (20 mL) was added nicotinaldehyde (341 mg, 3.19 mmol, 1.2 eq) and NaBH(OAc)₃ (1.13 g, 5.31 mmol, 2 eq). The mixture was stirred at 15° C. for 1 hour. Then the reaction mixture was quenched with water (80 mL) and extracted with EtOAc (6×100 mL). The combined organic layers were dried over Na₂SO₄, filtered and concentrated in vacua. The residue was purified by silica gel column chromatography (SiO₂, petroleum ether:ethyl acetate, 1:1 to 0:1) to give the title compound (1.1 g, 89%) as a yellow oil.

¹H NMR (DMSO-d₆) δ 8.53 (s, 1H), 8.46 (s, 1H), 7.72 (d, 1H), 7.37-7.33 (m, 1H), 7.13 (d, 4H), 6.88 (d, 4H), 4.21-4.17 (m, 5H), 3.73 (s, 6H), 3.61 (s, 2H), 3.47-3.41 (m, 2H) and 3.33-3.31 (m, 2H).

### Step B: 1-(Pyridin-3-ylmethyl)azetidine-3-sulfonamide

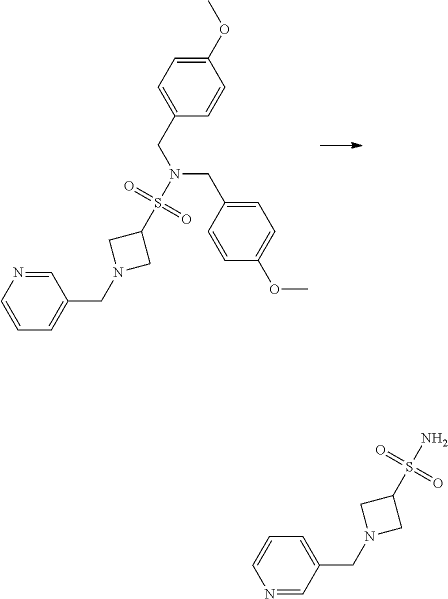 [SMILES: COc1ccc(CN(Cc2ccc(OC)cc2)S(=O)(=O)C2CN(Cc3cccnc3)C2)cc1 ; NS(=O)(=O)C1CN(Cc2cccnc2)C1]

A solution of N,N-bis(4-methoxybenzyl)-1-(pyridin-3-ylmethyl)azetidine-3-sulfonamide (1 g, 2.14 mmol, 1 eq) in TFA (10 mL) was stirred at to ° C. for 36 hours. Then the reaction mixture was concentrated in vacua. The residue was treated with MeOH (80 mL) and the mixture was stirred for another 1 hour. Then the mixture was filtered and the filtrate was concentrated in vacua. The residue was purified by reversed phase flash chromatography (water (0.1% of NH₃.H₂O)-MeCN) to give the title compound (240 mg, 49%) as a white solid.

¹H NMR (DMSO-d₆) δ 8.52-8.45 (m, 2H), 7.67 (d, 1H), 7.35 (dd, 1H), 6.98 (s, 2H), 3.99-3.94 (m, 1H), 3.64 (s, 2H), 3.54-3.49 (m, 2H) and 3.44-3.35 (m, 2H).

LCMS: m/z 228.1 (M+H)⁺ (ES^{+).}

### Intermediate P18: 1-Isopropylpiperidine-4-sulfonamide

### Step A: Benzyl 4-hydroxypiperidine-1-carboxylate

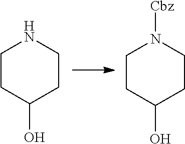 [SMILES: OC1CCNCC1 ; O=C(OCc1ccccc1)N1CCC(O)CC1]

To a solution of piperidin-4-ol (100 g, 988.66 mmol, 1 eq) in DCM (1 L) was added TEA (100.04 g, 988.66 mmol, 1 eq) and benzyl chloroformate (168.66 g, 988.66 mmol, 1 eq) at 0° C. The mixture was warmed to 25° C. and stirred for 12 hours. Then the reaction mixture was diluted with DCM (500 mL), washed with brine (3×500 mL), dried over Na₂SO₄, filtered and concentrated under reduced pressure to give the title compound (220 g, 95%) as a yellow oil, which was used in the next step without further purification.

¹H NMR (CDCl₃) δ 7.36-7.29 (m, 5H), 5.10 (s, 2H), 3.90-3.81 (m, 3H), 3.15-3.08 (m, 2H), 1.83-1.81 (m, 2H) and 1.47-1.45 (m, 2H). One exchangeable proton not observed.

LCMS: m/z 258.1 (M+Na)⁺ (ES^{+).}

### Step B: Benzyl 4-((methylsulfonyl)oxy)piperidine-1-carboxylate

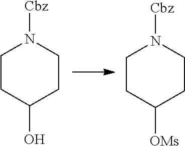 [SMILES: O=C(OCc1ccccc1)N1CCC(O)CC1 ; CS(=O)(=O)OC1CCN(C(=O)OCc2ccccc2)CC1]

To a solution of benzyl 4-hydroxypiperidine-1-carboxylate (220 g, 935.06 mmol, 1 eq) in DCM (1.7 L) was added TEA (189.24 g, 1.87 mol, 2 eq). Then mesyl chloride (128.54 g, 1.12 mol, 1.2 eq) was added dropwise at 0° C. The solution was heated to 25° C. and stirred for 1 hour. Then the reaction mixture was quenched with saturated aqueous NaHCO₃ solution (1.2 L) and the two layers were separated. The organic layer was washed with saturated aqueous NaHCO₃ solution (1.2 L) and brine (2×1 L), dried over anhydrous Na₂SO₄, filtered and concentrated in vacua to give the title compound (293 g, 100%), which was used directly in the next step.

### Step C: Benzyl 4-(acetylthio)piperidine-1-carboxylate

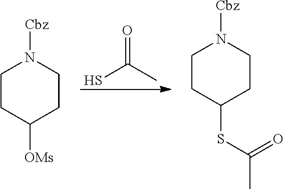

To a solution of benzyl 4-((methylsulfonyl)oxy)piperidine-1-carboxylate (290 g, 925.43 mmol, 1 eq) in DMF (1.4 L) was added Cs₂CO₃ (331.67 g, 1.02 mol, 1.1 eq) and ethanethioic S-acid (77.49 g, 1.02 mol, 1.1 eq). The mixture was stirred at 80° C. for 12 hours. Some solid was precipitated. The reaction mixture was filtered. The filtrate was concentrated in vacuo to remove most of the DMF. The residue was diluted with EtOAc (16 L), washed with H₂O (3×1 L) and brine (2×1 L), dried over anhydrous Na₂SO₄, filtered and concentrated in vacua. The residue was purified by silica gel column chromatography (SiO₂, petroleum ether:ethyl acetate, 50:1 to 40:1) to give the title compound (146 g, crude) as a yellow oil.

¹H NMR (CDCl₃) δ 7.37-7.35 (m, 5H), 5.13 (s, 2H), 4.07-3.93 (m, 2H), 3.66-3.61 (m, 1H), 3.19-3.12 (m, 2H), 2.33 (s, 3H), 1.94-1.91 (m, 2H) and 1.59-1.56 (m, 2H).

LCMS: m/z 294.1 (M+H)⁺ (ES⁺).

### Step D: Benzyl 4-(chlorosulfonyl)piperidine-1-carboxylate

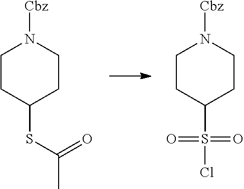 [SMILES: CC(=O)SC1CCN(C(=O)OCc2ccccc2)CC1 ; O=C(OCc1ccccc1)N1CCC(S(=O)(=O)Cl)CC1]

To a solution of benzyl 4-(acetylthio)piperidine-1-carboxylate (30.00 g, 102.26 mmol, 1 eq) in AcOH (1 L) and H₂O (100 mL) was added NCS (40.96 g, 306.77 mmol, 3 eq). The reaction mixture was stirred at 25° C. for 40 minutes. Then the reaction mixture was poured into water (1 L) and extracted with DCM (1 L). The organic layer was washed with water (3×1 L) and brine (1 L), dried over Na₂SO₄, and filtered to give the title compound in DCM (1 L) solution (theoretical amount: 32.4 g, crude), which was used in the next step without further purification.

### Step E: Benzyl 4-sulfamoylpiperidine-1-carboxylate

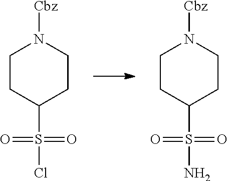 [SMILES: O=C(OCc1ccccc1)N1CCC(S(=O)(=O)Cl)CC1 ; NS(=O)(=O)C1CCN(C(=O)OCc2ccccc2)CC1]

NH₃ was bubbled into a solution of benzyl 4-(chlorosulfonyl)piperidine-1-carboxylate (theoretical amount: 30 g, crude) in DCM (1 L) at 0° C. for 20 minutes. Then the reaction mixture was stirred at 25° C. for 40 minutes. The reaction mixture was filtered and the filtrate was concentrated in vacua. The residue was triturated with a mixture of EtOAc (50 mL) and petroleum ether (40 mL) to give the title compound (21 g, 75%) as a yellow solid.

¹H NMR (DMSO-d₆) δ 7.38-7.32 (m, 5H), 6.79 (br s, 2H), 5.10 (s, 2H), 4.12-4.01 (m, 2H), 3.09-3.02 (m, 1H), 3.01-2.75 (m, 2H), 2.02-1.96 (m, 2H) and 1.51-1.41 (m, 2H).

### Step F: Piperidine-4-sulfonamide

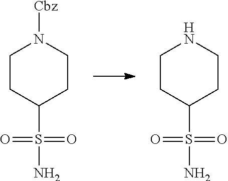 [SMILES: NS(=O)(=O)C1CCN(C(=O)OCc2ccccc2)CC1 ; NS(=O)(=O)C1CCNCC1]

To a solution of benzyl 4-sulfamoylpiperidine-1-carboxylate (21 g, 70.39 mmol, 1 eq) in MeOH (200 mL) was added Pd/C (10 wt % loading on activated carbon, 4 g) under nitrogen. The suspension was degassed in vacuo and purged with hydrogen several times. The mixture was stirred under hydrogen (50 psi) at 25° C. for 30 hours. Then the reaction mixture was filtered and the filtrate was concentrated in vacuo. The residue was triturated with EtOAc (200 mL) to give the title compound (11.2 g, 97% yield, 100% purity on LCMS) as a white solid.

¹H NMR (DMSO-d₆+D₂O) δ 3.06-2.90 (m, 2H), 2.89-2.86 (m, 1H), 2.50-2.46 (m, 2H), 1.95-1.91 (m, 2H) and 1.53-1.46 (m, 2H). Three exchangeable protons not observed.

LCMS: m/z 165.1 (M+H)⁺ (ES⁺).

### Step G: 1-Isopropylpiperidine-4-sulfonamide

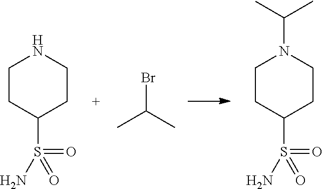

To a solution of piperidine-4-sulfonamide (1.2 g, 7.31 mmol, 1 eq) in acetonitrile (20 mL) was added 2-bromopropane (3.59 g, 29.23 mmol, 4 eq) and NaHCO₃ (1.84 g, 21.92 mmol, 3 eq). Then the reaction mixture was stirred at 70° C. for 18 hours. The hot mixture was filtered and the filtrate was concentrated in vacuo to give the title compound (1.05 g, 69% yield, 98.5% purity on LCMS) as a white solid.

¹H NMR (DMSO-d₆) δ 6.61 (s, 2H), 2.81-2.77 (m, 2H), 2.66-2.61 (m, 2H), 2.05-1.99 (m, 2H), 1.91-1.87 (m, 2H), 1.50-1.45 (m, 2H) and 0.89 (dd, 6H).

LCMS: m/z 207.1 (M+H)⁺ (ES⁺).

### Intermediate P19: (4-(Dimethylamino)pyridin-1-ium-1-carbonyl)((1-isopropyl-2-oxo-1,2-dihydropyrimidin-5-yl)sulfonyl)amide

### Step A: 5-Bromo-1-isopropylpyrimidin-2(1H)-one

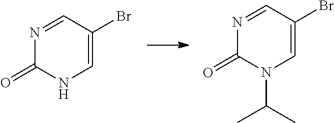

A suspension of 5-bromopyrimidin-2(1H)-one (10.07 g, 57.5 mmol) and K₂CO₃ (8.35 g, 60.4 mmol) in DMF (200 mL) was treated with 2-iodopropane (6.4 ml, 62.7 mmol) under nitrogen. The resultant suspension was stirred at room temperature for 40 hours, concentrated in vacua and the residue was partitioned between EtOAc (100 mL) and water (50 mL). The organic layer was collected and the aqueous layer was extracted with EtOAc (3×50 mL). The combined organic extracts were washed with 20% v/v brine (3×50 mL), brine (50 mL), dried (MgSO₄) and concentrated in vacua to afford crude product as a yellow oil (4.71 g). The crude product was purified by chromatography on silica gel (dry load) (40 g cartridge, 0-5% MeOH/DCM) to afford the title compound (1.34 g, 10%) as a clear yellow oil that solidified on standing.

¹H NMR (CDCl₃) δ 8.52 (dd, J=3.3, 1.6 Hz, 1H), 7.76 (d, J=3.2 Hz, 1H), 4.99 (pd, J=6.8, 1.6 Hz, 1H), 1.40 (dd, J=6.8, 1.0 Hz, 6H).

LCMS: m/z 217.0 (MBr⁷⁹+H)⁺ (ES^{+).}

### Step B: 5-(Benzylthio)-1-isopropylpyrimidin-2(1H)-one

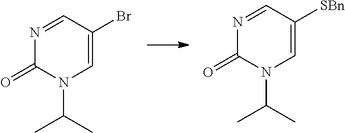

A solution of 5-bromo-1-isopropylpyrimidin-2(1H)-one (1.217 g, 5.05 mmol), DIPEA (1.8 ml, 10.31 mmol) and benzyl mercaptan (0.6 ml, 5.07 mmol) in dioxane (25 mL) was sparged with nitrogen for 15 minutes before Pd₂(dba)₃ (0.233 g, 0.254 mmol) and Xantphos (0.294 g, 0.508 mmol) were added. The reaction mixture was heated at 100° C. for 22 hours and then concentrated in vacua. The residue was partitioned between EtOAc (30 mL) and saturated aqueous NaHCO₃ (20 mL). The aqueous layer was extracted with EtOAc (3×30 mL) and the combined organic extracts were washed with brine (30 mL), dried (MgSO₄) and concentrated in vacuo to afford crude product as a brown oil (2.3 g). The crude product was purified by chromatography on silica gel (dry load) (40 g cartridge, 0-5% MeOH/DCM) to afford the title compound (1.49 g, 99%) as a brown oil.

¹H NMR (CDCl₃) δ 8.46 (d, J=3.1 Hz, 1H), 7.30-7.22 (m, 3H), 7.15 (d, J=3.2 Hz, 1H), 7.09-7.06 (m, 2H), 4.84 (sept, J=6.8 Hz, 1H), 3.80 (s, 2H), 1.13 (d, J=6.8 Hz, 6H).

LCMS; m/z 261.1 (M+H)⁺ (ES^{+).}

### Step C: 1-Isopropyl-N,N-bis(4-methoxybenzyl)-2-oxo-1,2-dihydropyrimidine-5-sulfonamide

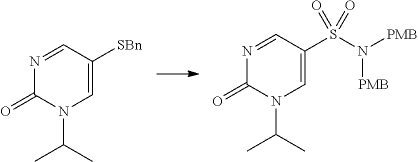

A suspension of 5-(benzylthio)-1-isopropylpyrimidin-2(1H)-one (1.012 g, 3.69 mmol) in DCM (15 mL) and water (1.5 mL) at 0° C. was treated with SO₂Cl₂ (2 ml, 23.86 mmol) dropwise. The resultant yellow suspension was stirred at 0° C. for 1 hour. A slurry of ice/water (20 mL) was added and the organic phase was collected and retained. The aqueous layer was extracted with DCM (2×10 mL) and the combined organic extracts were dried (MgSO₄) and concentrated in vacuo to afford crude sulfonyl chloride intermediate as a pale yellow liquid (1.024 g) which was used without further purification. A solution of bis(4-methoxybenzyl)amine (1.007 g, 3.91 mmol) and Et₃N (0.6 ml, 4.30 mmol) in DCM (20 mL) at 0° C. was treated with a solution of the crude sulfonyl chloride intermediate in DCM (10 mL). The resultant solution was allowed to warm to room temperature, stirred for 1 hour and then diluted with DCM (20 mL) and saturated aqueous NH₄Cl (20 mL). The organic layer was collected and washed with saturated aqueous NH₄Cl (20 mL) and water (20 mL), dried (MgSO₄) and concentrated in vacua to afford crude product as an orange oil (2.0 g). The crude product was triturated with TBME (30 mL), filtered, rinsing with TBME, and dried in vacua to afford crude product which was purified by chromatography on silica gel (24 g cartridge, 0-5% MeOH/DCM) to afford the title compound (0.941 g, 44%) as a sticky orange oil.

¹H NMR (CDCl₃) δ 8.65 (d, J=3.3 Hz, 1H), 7.96 (d, J=3.3 Hz, 1H), 7.15-7.10 (m, 4H), 6.85-6.82 (m, 4H), 4.88 (sept, J=6.8 Hz, 1H), 4.32 (s, 4H), 3.79 (s, 6H), 1.34 (d, J=6.8 Hz, 6H).

LCMS: m/z 458.1 (M+H)⁺ (ES⁺).

### Step D: 1-Isopropyl-2-oxo-1,2-dihydropyrimidine-5-sulfonamide

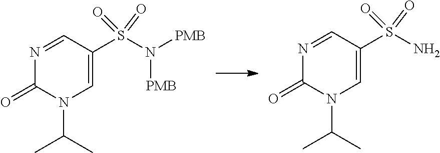

1-Isopropyl-N,N-bis(4-methoxybenzyl)-2-oxo-1,2-dihydropyrimidine-5-sulfonamide (0.941 g, 1.625 mmol) was treated with TFA (15 ml, 195 mmol) and the resultant solution was stirred at room temperature for 64 hours. Then the reaction mixture was concentrated in vacua and the crude product was purified by chromatography on silica gel (dry load) (12 g cartridge, 0-10% MeOH/DCM) to afford the title compound (0.350 g, 94%) as a tan solid.

¹H NMR (DMSO-d6) δ 8.81 (d, J=3.2 Hz, 1H), 8.51 (d, J=3.3 Hz, 1H), 7.45 (s, 2H), 4.77 (sept, J=6.8 Hz, 1H), 1.37 (d, J=6.8 Hz, 6H).

LCMS; m/z 218.1 (M+H)⁺ (ES⁺); 215.8 (M−H)⁻ (ES⁻).

### Step E: (4-(Dimethylamino)pyridin-1-ium-1-carbonyl)((1-isopropyl-2-oxo-1,2-dihydropyrimidin-5-yl)sulfonyl)amide

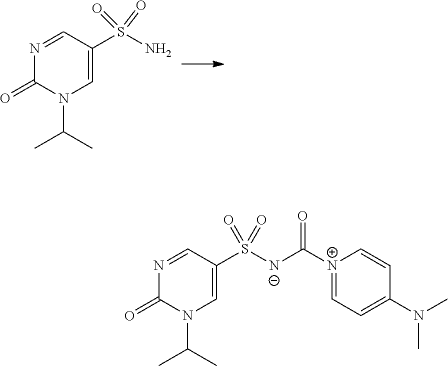 [SMILES: CC(C)n1cc(S(N)(=O)=O)cnc1=O ; CC(C)n1cc(S(=O)(=O)[N-]C(=O)[n+]2ccc(N(C)C)cc2)cnc1=O]

A suspension of 1-isopropyl-2-oxo-1,2-dihydropyrimidine-5-sulfonamide (0.150 g, 0.690 mmol) and DMAP (0.169 g, 1.383 mmol) in dry MeCN (2 mL) was stirred at room temperature for 10 minutes before diphenyl carbonate (0.163 g, 0.761 mmol) was added in one portion. The reaction was stirred for 18 hours, diluted with TBME (20 mL) and DCM (2 mL), and the precipitate was collected by filtration and used crude in the next step.

### Intermediate P20: 1-Isopropyl-2-oxo-1,2-dihydropyridine-4-sulfonamide

### Step A: Lithium 2-chloropyridine-4-sulfinate

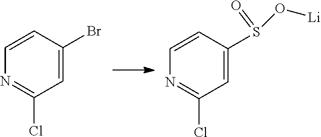

A solution of 4-bromo-2-chloropyridine (5.8 ml, 52.3 mmol) in dry THF (100 mL) at −78° C. was treated with 2.5 M BuLi (in hexanes) (22 ml, 55.0 mmol) dropwise under nitrogen. The resultant solution was stirred at −78° C. for 10 minutes and then SO₂ gas was bubbled through the solution for 20 minutes. The reaction was allowed to warm to room temperature and then concentrated in vacua. The residue was triturated with TBME (100 mL). The resultant solid was filtered, rinsing with TBME, and dried in vacua to afford the title compound (8.80 g, 92%) as a dark purple solid that was used crude in the next step.

### Step B: 2-Chloro-N,N-bis(4-methoxybenzyl)pyridine-4-sulfonamide

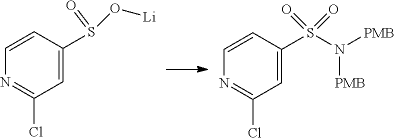

A suspension of lithium 2-chloropyridine-4-sulfinate (6.55 g, 35.7 mmol) in DCM (100 mL) at 0° C. was treated with NCS (4.862 g, 35.7 mmol) in one portion. The resultant suspension was stirred at 0° C. for 2 hours, quenched with water (50 mL) and the organic layer was collected. The aqueous layer was extracted with DCM (2×50 mL) and the combined organic extracts were washed with water (50 mL), dried (MgSO₄) and concentrated in vacuo to afford the crude sulfonyl chloride intermediate. A solution of the sulfonyl chloride intermediate in DCM (10 mL) was added dropwise to a suspension of bis(4-methoxybenzyl)amine (9.42 g, 36.6 mmol) and triethylamine (15.92 ml, 114 mmol) in DCM (100 mL) at 0° C. The reaction mixture was allowed to warm to room temperature, stirred for 16 hours and then water (100 mL) was added. The organic layer was collected and the aqueous layer was extracted with DCM (2×50 mL). The combined organic extracts were washed with water (100 mL), 1 M HCl (aq) (2×100 mL), water (100 mL), dried (MgSO₄) and concentrated in vacua to afford crude product which was purified by chromatography on silica gel (dry load) (80 g cartridge, 0-50% EtOAc/isohexane) to afford the title compound (0.677 g, 4%) as an orange solid.

¹H NMR (CDCl₃) δ 8.51 (dd, J=4.8, 1.9 Hz, 1H), 8.30 (dd, J=7.8, 1.9 Hz, 1H), 7.30 (dd, J=7.8, 4.8 Hz, 1H), 7.04-6.99 (m, 4H), 6.81-6.75 (m, 4H), 4.38 (s, 4H), 3.78 (s, 6H).

LCMS: m/z 433 (MCl³⁵⁺+H)⁺ (ES⁺).

### Step C: N,N-Bis(4-methoxybenzyl)-2-oxo-1,2-dihydropyridine-4-sulfonamide

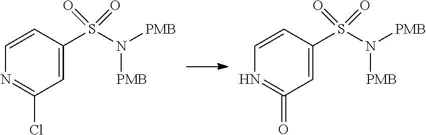

A suspension of 2-chloro-N,N-bis(4-methoxybenzyl)pyridine-4-sulfonamide (0.365 g, 0.759 mmol) in ethane-1,2-diol (5 ml, 0.759 mmol) was treated with 2 M KOH (aq) (1.9 ml, 3.80 mmol). The resultant suspension was stirred at 140° C. for 72 hours, allowed to cool to room temperature and then diluted with saturated aqueous NH₄Cl (30 mL) and EtOAc (20 mL). The organic layer was collected and the aqueous layer was extracted with EtOAc (2×20 mL). The combined organic extracts were dried (MgSO₄) and concentrated in vacua to afford crude product as a yellow solid (510 mg). The crude product was purified by chromatography on silica gel (dry load) (12 g cartridge, 0-100% EtOAc/isohexane) to afford the title compound (0.437 g, 68%) as a pale yellow solid.

LCMS: m/z 437.3 (M+Na)⁺ (ES⁺); 413.1 (M−H)⁻ (ES⁻).

### Step D: 1-Isopropyl-N,N-bis(4-methoxybenzyl)-2-oxo-1,2-dihydropyridine-4-sulfonamide

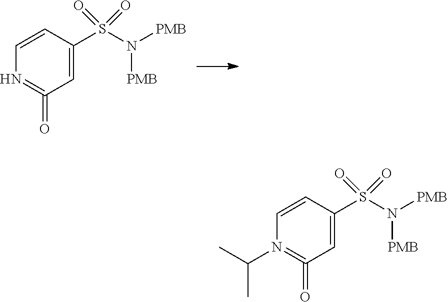

A suspension of N,N-bis(4-methoxybenzyl)-2-oxo-1,2-dihydropyridine-4-sulfonamide (0.437 g, 0.949 mmol) and lithium bromide (0.171 g, 1.930 mmol) in DME:DMF (7.5 mL, 4:1) at 0° C. was treated with NaH in one portion. The resultant suspension was stirred at 0° C. for 15 minutes, treated with 2-iodopropane (0.194 ml, 1.898 mmol) and heated to 65° C. for 65 hours. Further lithium bromide (0.171 g, 1.930 mmol) followed by NaH (0.053 g, 1.328 mmol) were added and the reaction mixture was stirred at 65° C. for 10 minutes. Then further 2-iodopropane (0.194 ml, 1.898 mmol) was added and the reaction mixture was stirred at 65° C. for 18 hours. EtOAc (10 mL) and saturated aqueous NH₄Cl (5 mL) were added and the organic layer was collected. The aqueous layer was extracted with EtOAc (2×10 mL) and the combined organic extracts were washed with 20% v/v brine (3×10 mL) and brine (10 mL), dried (MgSO₄) and concentrated in vacuo to afford crude product as a yellow oil. The crude product was purified by chromatography on silica gel (dry load) (12 g cartridge, 0-100% EtOAc/isohexane) to afford the title compound (0.385 g, 77%) as a pale yellow oil.

¹H NMR (DMSO-d6) δ 8.06 (dd, J=6.8, 2.1 Hz, 1H), 7.99 (dd, J=7.2, 2.0 Hz, 1H), 7.07-7.03 (m, 4H), 6.82-6.78 (m, 4H), 6.39 (t, J=7.0 Hz, 1H), 4.99 (sept, J=6.8 Hz, 1H), 4.34 (s, 4H), 3.71 (s, 6H), 1.28 (d, J=6.8 Hz, 6H).

LCMS; m/z 479.3 (M+Na)⁺ (ES⁺).

### Step E: 1-Isopropyl-2-oxo-1,2-dihydropyridine-4-sulfonamide

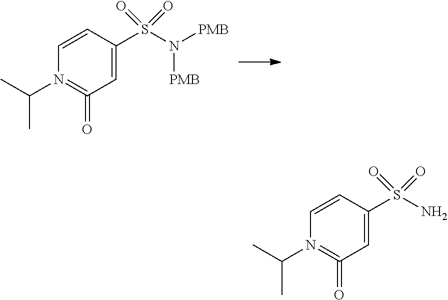

1-Isopropyl-N,N-bis(4-methoxybenzyl)-2-oxo-1,2-dihydropyridine-4-sulfonamide (0.375 g, 0.715 mmol) was treated with TFA (2 ml, 26.0 mmol) and the resultant red solution was stirred at room temperature for 17 hours. The reaction mixture was concentrated in vacuo, azeotroped with DCM (2×5 mL) and the crude product was purified by chromatography on silica gel (dry load) (4 g cartridge, 0-10% MeOH/DCM) to afford the title compound (0.160 g, 100%) as a white solid.

¹H NMR (CDCl₃) δ 8.09 (dd, J=7.1, 2.1 Hz, 1H), 7.61 (dd, J=6.9, 2.1 Hz, 1H), 6.42 (t, J=7.0 Hz, 1H), 5.38 (br s, 2H), 5.32 (sept, J=7.0 Hz, 1H), 1.41 (d, J=6.8 Hz, 6H).

LCMS: m/z 217.3 (M+H)⁺ (ES⁺); 215.1 (M−H)⁻ (ES⁻).

### Intermediate P21: 6-(Dimethylamino)pyrazine-2-sulfonamide

### Step A: 2-(Benzylthio)-6-chloropyrazine

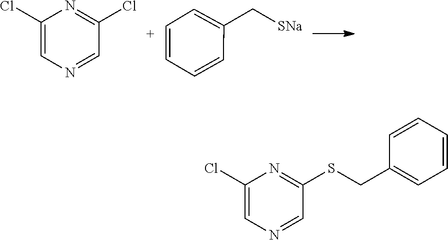

A solution of 2,6-dichloropyrazine (5 g, 33.56 mmol, 1.1 eq) and sodium phenylmethanethiolate (4.46 g, 30.51 mmol, 1 eq) in DMF (50 mL) was stirred at 25° C. for 16 hours. The reaction mixture was diluted with EtOAc (100 mL) and washed with saturated aqueous NH₄Cl solution (3×50 mL) and brine (3×50 mL). The organic layers were dried over anhydrous Na₂SO₄, filtered and concentrated in vacuo. The residue was purified by silica gel column chromatography (SiO₂, petroleum ether:ethyl acetate, 1:0 to 50:1) to give the title compound (2 g, 28%) as a colourless oil.

¹H NMR (CDCl₃): δ 8.33 (d, 1H), 8.23 (s, 1H), 7.46-7.42 (m, 2H), 7.37-7.29 (m, 3H) and 4.43 (s, 2H).

LCMS: m/z 237.0 (M+H)⁺ (ES⁺).

### Step B: 6-Chloropyrazine-2-sulfonyl chloride

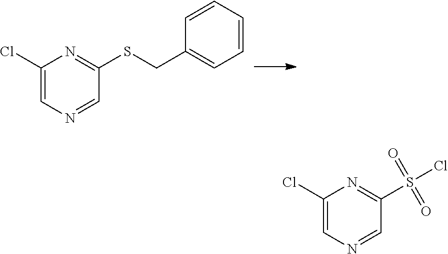 [SMILES: Clc1cncc(SCc2ccccc2)n1 ; O=S(=O)(Cl)c1cncc(Cl)n1]

To a solution of 2-(benzylthio)-6-chloropyrazine (2 g, 8.45 mmol, 1 eq) in CCl₄ (80 mL) and H₂O (20 mL) was bubbled with C12 at 0° C. for 10 minutes. The reaction mixture was filtered and the filtrate was concentrated in vacua to give the title compound (1.8 g, crude), which was used directly in the next step.

### Step C: 6-Chloropyrazine-2-sulfonamide

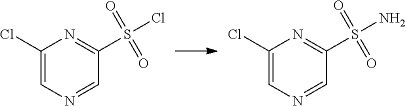 [SMILES: O=S(=O)(Cl)c1cncc(Cl)n1 ; NS(=O)(=O)c1cncc(Cl)n1]

To a solution of 6-chloropyrazine-2-sulfonyl chloride (1.8 g, crude) in THF (50 mL) was bubbled with NH₃ at 0° C. for 10 minutes. The reaction mixture was filtered and the filtrate was concentrated in vacuo. The residue was triturated with a mixture of petroleum ether and ethyl acetate (21 mL, v:v=20:1) to give the title compound (1.2 g, 73%) as a yellow solid.

¹H NMR (DMSO-d₆): δ 9.09 (d, 2H) and 7.96 (s, 2H).

### Step D: 6-(Dimethylamino)pyrazine-2-sulfonamide

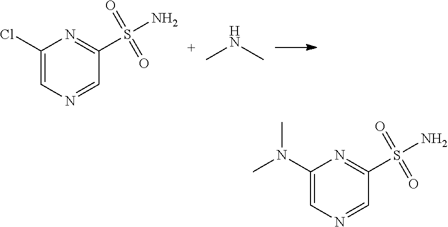

To a solution of 6-chloropyrazine-2-sulfonamide (1 g, 5.16 mmol, 1 eq) in MeCN (10 mL) was added with dimethylamine (2 M in THF, 3.23 mL, 1.25 eq). The mixture was stirred at 25° C. for 3 hours. The reaction mixture was concentrated in vacua. The residue was purified by silica gel column chromatography (SiO₂, petroleum ether:ethyl acetate, 1:1 to 1:10) to give the title compound (210 mg, 20%) as a yellow solid.

¹H NMR (CD₃OD): δ 8.26 (s, 1H), 8.22 (s, 1H) and 3.22 (s, 6H).

LCMS: m/z 203.1 (M+H)⁺ (ES⁺).

### Intermediate P22: 5-(Dimethylamino)pyrazine-2-sulfonamide

### Step A: 2-(Benzylthio)-5-chloropyrazine

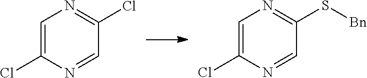 [SMILES: Clc1cnc(Cl)cn1 ; Clc1cnc(SCc2ccccc2)cn1]

To a solution of 2,5-dichloropyrazine (3 g, 20.14 mmol, 1 eq) in MeCN (30 mL) was added phenylmethanethiol (2.25 g, 18.12 mmol, 0.9 eq) and K₂CO₃ (5.57 g, 40.27 mmol, 2 eq). The reaction mixture was stirred at 25° C. for 12 hours. The reaction mixture was poured into water (100 mL) and extracted with EtOAc (2×100 mL). The combined organic layers were dried over Na₂SO₄, filtered and concentrated in vacua. The residue was purified by silica gel column chromatography (SiO₂, petroleum ether:ethyl acetate, 10:1 to 0:1) to give the title compound (4.5 g, 94%) as a yellow oil.

¹H NMR (CDCl₃): δ 8.43 (s, 1H), 8.19 (s, 1H), 7.42-7.38 (m, 2H), 7.35-7.28 (m, 3H) and 4.42 (s, 2H).

### Step B: 5-Chloropyrazine-2-sulfonyl chloride

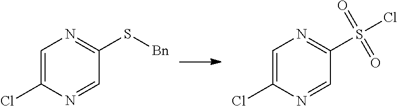 [SMILES: Clc1cnc(SCc2ccccc2)cn1 ; O=S(=O)(Cl)c1cnc(Cl)cn1]

Cl₂ (15 psi) was bubbled into a solution of 2-(benzylthio)-5-chloropyrazine (4.5 g, 19.01 mmol, 1 eq) in CCl₄ (50 mL) and H₂O (10 mL) at −10° C. for 15 minutes. The reaction mixture was used directly in the next step without further work-up and purification.

### Step C: 5-Chloropyrazine-2-sulfonamide

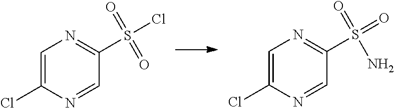 [SMILES: O=S(=O)(Cl)c1cnc(Cl)cn1 ; NS(=O)(=O)c1cnc(Cl)cn1]

A saturated solution of NH₃ in THF (20 mL) was added into a solution of 5-chloropyrazine-2-sulfonyl chloride (theoretical amount: 4 g, crude) in CCl₄ (50 mL) and H₂O (10 mL) at −10° C. for 10 minutes. Then the reaction mixture was warmed to 25° C. and stirred at 25° C. for 50 minutes. The reaction mixture was concentrated in vacuo. The residue was purified by silica gel column chromatography (SiO₂, petroleum ether:ethyl acetate, 30:1 to 1:1) to give the title compound (1.6 g, 44%) as a yellow oil.

¹H NMR (CDCl₃): δ 8.98 (dd, 1H) and 7.88 (s, 1H).

### Step D: 5-(Dimethylamino)pyrazine-2-sulfonamide

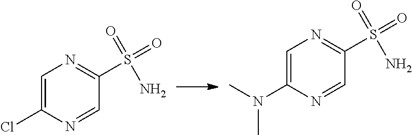

5-Chloropyrazine-2-sulfonamide (800 mg, 4.13 mmol, 1 eq) was added into a solution of dimethylamine in water (2 M, 10.00 mL, 33 wt % in H₂O, 4.84 eq). Then the mixture was stirred at 25° C. for 30 minutes. The reaction mixture was concentrated under reduced pressure. The residue was triturated with EtOAc (30 mL) to give the title compound (800 mg, 96%) as a white solid.

¹H NMR (DMSO-d₆): δ 8.46 (s, 1H), 8.20 (s, 1H), 7.28 (s, 2H) and 3.17 (s, 6H).

### Intermediate P23: 3-(Difluoromethyl)pyrazine-2-sulfonamide

### Step A: 3-Chloropyrazine-2-carbaldehyde

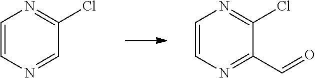 [SMILES: Clc1cnccn1 ; O=Cc1nccnc1Cl]

To a solution of 2,2,6,6-tetramethylpiperidine (27.13 g, 192.08 mmol, 2.2 eq) in THF (200 mL) was added n-BuLi (2.5 M, 73.34 mL, 2.1 eq) at −78° C. The reaction mixture was warmed to 0° C. and stirred for 15 minutes. Then the reaction mixture was cooled down to −78° C. and 2-chloropyrazine (10 g, 87.31 mmol, 1 eq) was added. The resulting mixture was stirred at −78° C. for 30 minutes. To the reaction mixture was added DMF (12.76 g, 174.62 mmol, 2 eq) at −78° C. The mixture was stirred at −78° C. for 30 minutes and then stirred at 0° C. for another 15 minutes. The reaction mixture was quenched with a solution of AcOH (50 mL) in THF (50 mL) at −78° C. Then the reaction mixture was poured into water (300 mL) and extracted with EtOAc (3×300 mL). The combined organic layers were washed with brine, dried over Na₂SO₄, filtered and concentrated in vacuo. The residue was purified by silica gel column chromatography (SiO₂, petroleum ether:ethyl acetate, 10:1 to 5:1) to give the title compound (2.4 g, 19%) as a yellow oil.

¹H NMR (CDCl₃): δ 10.35 (s, 1H), 8.78-8.72 (m, 1H) and 8.62-8.58 (m, 1H).

### Step B: 2-Chloro-3-(difluoromethyl)pyrazine

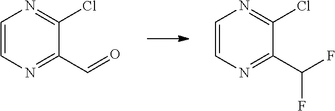 [SMILES: O=Cc1nccnc1Cl ; FC(F)c1nccnc1Cl]

To a solution of 3-chloropyrazine-2-carbaldehyde (1.2 g, 8.42 mmol, 1 eq) in DCM (so mL) was added bis(2-methoxyethyl)aminosulfur trifluoride (2.79 g, 12.63 mmol, 1.5 eq) at −78° C. The mixture was warmed to 25° C. and stirred for 2 hours. The reaction mixture was quenched with water (50 mL) and extracted with DCM (3×80 mL). The combined organic layers were dried over Na₂SO₄, filtered and concentrated in vacua. The residue was purified by silica gel column chromatography (SiO₂, petroleum ether:ethyl acetate, 1:0 to 10:1) to give the title compound (800 mg, 58%) as a yellow oil.

¹H NMR (CDCl₃): δ 8.54 (d, 1H), 8.47 (d, 1H) and 6.85 (t, 1H).

### Step C: 2-(Benzylthio)-3-(difluoromethyl)pyrazine

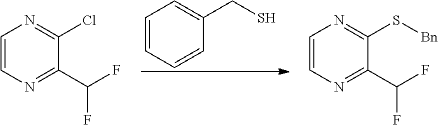

To a solution of 2-chloro-3-(difluoromethyl)pyrazine (800 mg, 4.86 mmol, 1 eq) in MeCN (15 mL) was added phenylmethanethiol (664 mg, 5.35 mmol, 1.1 eq) and K₂CO₃ (874 mg, 6.32 mmol, 1.3 eq). The mixture was stirred at 25° C. for 12 hours. Then the reaction mixture was poured into water (50 mL) and extracted with EtOAc (2×50 mL). The combined organic layers were dried over Na₂SO₄, filtered and concentrated in vacua. The residue was purified by silica gel column chromatography (SiO₂, petroleum ether: ethyl acetate, 1:0 to 10:1) to give the title compound (1.1 g, 90%) as a colourless oil.

¹H NMR (CDCl₃): δ 8.56-8.52 (m, 1H), 8.33 (d, 1H), 7.45-7.42 (m, 2H), 7.36-7.30 (m, 3H), 6.71 (t, 1H) and 4.51 (s, 2H).

### Step D: 3-(Difluoromethyl)pyrazine-2-sulfonyl chloride

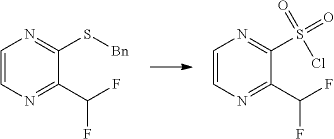 [SMILES: FC(F)c1nccnc1SCc1ccccc1 ; O=S(=O)(Cl)c1nccnc1C(F)F]

Cl₂ (15 psi) was bubbled into a solution of 2-(benzylthio)-3-(difluoromethyl)pyrazine (500 mg, 1.98 mmol, 1 eq) in DCM (20 mL) and H₂O (2 mL) at −10° C. for 5 minutes. The reaction mixture was used directly in the next step without purification.

### Step E: 3-(Difluoromethyl)pyrazine-2-sulfonamide

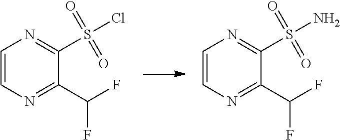 [SMILES: O=S(=O)(Cl)c1nccnc1C(F)F ; NS(=O)(=O)c1nccnc1C(F)F]

To a solution of 3-(difluoromethyl)pyrazine-2-sulfonyl chloride (theoretical amount: 453 mg, crude) in DCM (20 mL) and H₂O (2 mL) was added NH₃.H₂O (15 mL, 25 wt % in water) at 0° C. The reaction mixture was stirred at 0° C. for 5 minutes and then concentrated in vacuo. The residue was treated with water (50 mL) and the mixture was washed with EtOAc (3×80 mL). The aqueous layer was concentrated in vacua. The residue was treated with EtOAc (100 mL) and the mixture was stirred for 10 minutes. The mixture was filtered and the filtrate was concentrated in vacuo to give the title compound (260 mg, 63%) as a yellow oil.

¹H NMR (DMSO-d₆): δ 9.08 (d, 1H), 9.02 (s, 1H), 8.10 (br 5, 2H) and 7.52 (t, 1H).

LCMS: m/z 210.1 (M+H)⁺ (ES⁺).

### Intermediate P24: 4,6-Dimethylpyrimidine-2-sulfonamide

### Step A: 4,6-Dimethylpyrimidine-2-thiol and 1,2-bis(4,6-dimethylpyrimidin-2-yl)disulfane

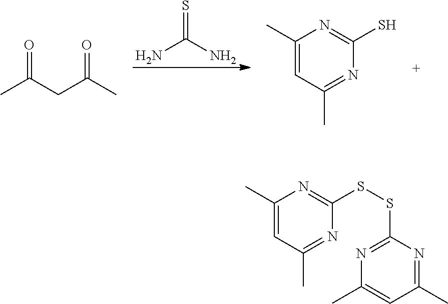

To a solution of pentane-2,4-dione (10.03 g, 100.17 mmol, 1.25 eq) in concentrated HCl solution (12 M, 20 mL, 2.99 eq) and EtOH (100 mL) was added thiourea (6.1 g, 80.14 mmol, 1 eq) at 10° C. The reaction mixture was stirred at 70° C. for 2 hours. The reaction mixture was cooled to 20° C. and a large amount of solid precipitated out. The mixture was filtered and the filter cake was treated with saturated aqueous NaHCO₃ solution (300 mL). The mixture was filtered again and the filter cake was triturated with MeOH (200 mL) to give the title compound (10.3 g, 44% yield, 97.2% purity on LCMS) as a yellow solid.

¹H NMR (DMSO-d₆): δ 6.39 (s, 2H) and 2.13 (s, 12H).

LCMS: m/z 279.1 (M+H)⁺ (ES⁺).

### Step B: 4,6-Dimethylpyrimidine-2-sulfonyl chloride

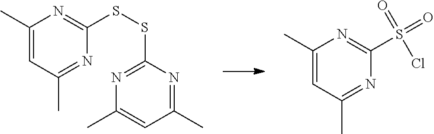

Cl₂ (15 psi) was bubbled into a solution of 1,2-bis(4,6-dimethylpyrimidin-2-yl)disulfane (1 g, 3.59 mmol, 1 eq) in DCM (40 mL) and H₂O (6 mL) at −10° C. for 10 minutes. The reaction mixture was quenched with water (20 mL) and extracted with DCM (2×40 mL). The solution of the title compound (crude) in DCM (80 mL) was used directly in the next step without further purification.

### Step C: 4,6-Dimethylpyrimidine-2-sulfonamide

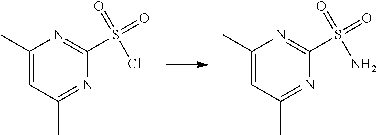

NH₃ (15 psi) was bubbled into a solution of 4,6-dimethylpyrimidine-2-sulfonyl chloride (theoretical amount: 0.74 g, crude) in DCM (80 mL) at 0° C. for 10 minutes. The reaction mixture was quenched with water (20 mL) and washed with DCM (40 mL). Then the aqueous phase was concentrated in vacua. The residue was triturated with EtOAc (300 mL) to give the title compound (0.35 g, 52% yield, 100% purity on LCMS) as a yellow solid.

¹H NMR (DMSO-d₆): δ 7.49-7.47 (m, 3H) and 2.52 (s, 6H).

LCMS: m/z 188.1 (M+H)⁺ (ES⁺).

### Intermediate P25: 5-(Dimethylamino)pyridazine-3-sulfonamide

### Step A: 6-Chloro-N,N-dimethylpyridazin-4-amine

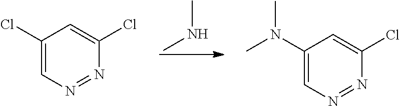

To a mixture of 3,5-dichloropyridazine (13.5 g, 90.62 mmol, 1 eq) in THF (100 mL) was added dimethylamine (270 mL, 543.70 mmol, in THF solution, 6 eq) in one portion at 25° C. Then the reaction mixture was stirred at 25° C. for 12 hours. The reaction mixture was concentrated in vacua. The residue was purified by reversed phase flash chromatography (0.05% of NH₃.H₂O in water/MeCN) to give the title compound (7 g, 49% yield, 99.35% purity on LCMS) as a brown solid.

¹H NMR (CDCl₃): δ 8.63 (d, 1H), 6.53 (d, 1H) and 3.09 (s, 6H).

LCMS: m/z 158.1 (M+H)⁺ (ES⁺).

### Step B: 6-(Benzylthio)-N,N-dimethylpyridazin-4-amine

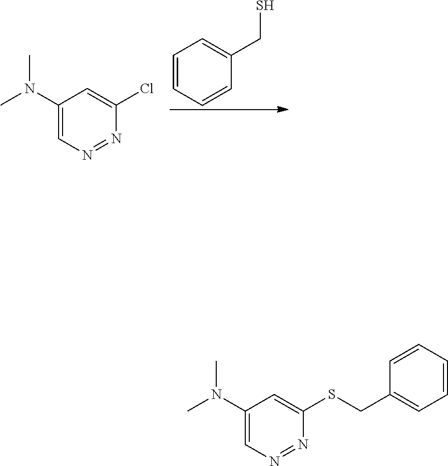

To a mixture of phenylmethanethiol (4.31 g, 34.70 mmol, 1.22 eq) in DMF (100 mL) was added NaH (1.37 g, 34.26 mmol, 60 wt % in mineral oil, 1.2 eq) at 0° C. in one portion under N₂. Then mixture was stirred at 0° C. for 0.5 hour. Then 6-chloro-N,N-dimethylpyridazin-4-amine (4.5 g, 28.55 mmol, 1 eq) was added. The reaction mixture was heated to 70° C. and stirred for 1 hour. Then the reaction mixture was quenched with water (200 mL) and extracted with EtOAc (3×200 mL). The combined organic phases were washed with brine (200 mL), dried over anhydrous Na₂SO₄, filtered and concentrated in vacuo. The residue was purified by silica gel column chromatography (SiO₂, petroleum ether:ethyl acetate, 1:0 to 20:1, then flushed through with EtOAc:EtOH, 50:1 to 10:1) to give the title compound (5.2 g, 74%) as a brown solid.

¹H NMR (CDCl₃): δ 8.53 (d, 1H), 7.45-7.43 (m, 2H), 7.32-7.30 (m, 2H), 7.26-7.23 (m, 1H), 6.34 (d, 1H), 4.58 (s, 2H) and 3.09 (s, 6H).

### Step C: 5-(Dimethylamino) pyridazine-3-sulfonyl chloride

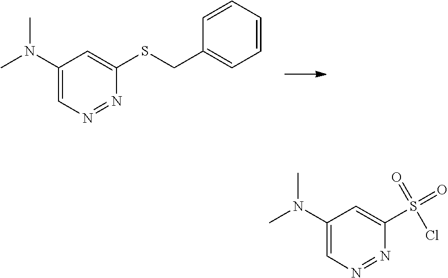 [SMILES: CN(C)c1cnnc(SCc2ccccc2)c1 ; CN(C)c1cnnc(S(=O)(=O)Cl)c1]

To a solution of 6-(benzylthio)-N,N-dimethylpyridazin-4-amine (1 g, 4.08 mmol, 1 eq) in DCM (50 mL) was added a solution of CaCl₂) (4.52 g, 40.76 mmol, 10 eq) in HCl (1 M, 20.38 mL, 5 eq) at −30° C. Then a solution of CaCl₂) (14.70 g, 132.47 mmol, 32.5 eq) in aqueous NaClO solution (19.22 g, 15.49 mmol, 6 wt % in water, 3.8 eq) was added dropwise at −30° C. The resulting mixture was stirred at −30° C. for 30 minutes. The reaction mixture was quenched with water (20 mL) and extracted with DCM (2×50 mL). The combined organic phases were dried over anhydrous Na₂SO₄, filtered and concentrated in vacua to give a solution of the title compound (theoretical amount: 0.9 g, crude) in DCM (100 mL), which was used directly in the next step without further purification.

### Step D: 5-(Dimethylamino) pyridazine-3-sulfonamide

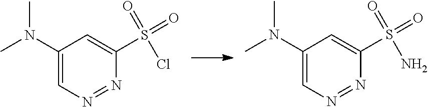

NH₃ (15 psi) was bubbled into a solution of 5-(dimethylamino)pyridazine-3-sulfonyl chloride (theoretical amount: 0.9 g, crude) in DCM (100 mL) at −20° C. for 10 minutes. The mixture was quenched with water (50 mL) and washed with DCM (30 mL). Then the aqueous phase (50 mL) was concentrated in vacua. The residue was purified by trituration with EtOAc (300 mL) to give the title compound (0.23 g, 28%) as a yellow solid.

¹H NMR (DMSO-d₆): δ 8.89 (d, 1H), 7.55 (s, 2H), 7.05 (d, 1H) and 3.09 (s, 6H).

LCMS: m/z 203.1 (M+H)⁺ (ES⁺).

### Intermediate P26: 2-Methylpropane-1-sulfonamide

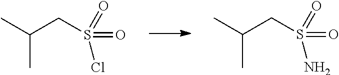

A solution of 2-methylpropane-1-sulfonyl chloride (1.5 g, 9.58 mmol, 1 eq) in THF (20 mL) was cooled to 0° C. Then NH₃ (15 psi) was bubbled into the mixture at 0° C. for 10 minutes. The mixture was stirred at 0° C. for another 10 minutes. The reaction mixture was filtered and the filtrate was concentrated in vacuo to give the title compound (1 g, 76%) as a colourless oil.

¹H NMR (DMSO-d₆): δ 6.72 (s, 2H), 2.86 (d, 2H), 2.19-2.07 (m, 1H) and 1.01 (d, 6H).

### Intermediate P27: 2-Phenylethanesulfonamide

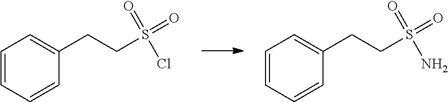 [SMILES: O=S(=O)(Cl)CCc1ccccc1 ; NS(=O)(=O)CCc1ccccc1]

NH₃ was bubbled into THF (10 mL) at −78° C. for 5 minutes. Then a solution of 2-phenylethanesulfonyl chloride (0.5 g, 2.44 mmol, 1 eq) in THF (10 mL) was added to the NH₃/THF solution at 25° C. The resulting mixture was stirred for 12 minutes. The mixture was filtered and the filtrate was concentrated in vacuo to give the title compound (0.38 g, 84%) as a white solid.

¹H NMR (CDCl₃): δ 7.38-7.33 (m, 2H), 7.29-7.24 (m, 3H), 4.42 (br s, 2H), 3.45-3.40 (m, 2H) and 3.22-3.17 (m, 2H).

LCMS: m/z 208.1 (M+Na)⁺ (ES⁺).

### Intermediate P28: 1-Phenylethanesulfonamide

### Step A: N,N-Bis(4-methoxybenzyl)-1-phenylmethanesulfonamide

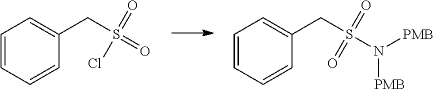

To a solution of bis(4-methoxybenzyl)amine (4.05 g, 15.74 mmol, 1 eq) in DCM (40 mL) was added TEA (3.18 g, 31.47 mmol, 2 eq) and phenylmethanesulfonyl chloride (3 g, 15.74 mmol, eq). The mixture was stirred at 20° C. for 12 hours. The reaction mixture was concentrated in vacuo. The residue was treated with water (50 mL) and extracted with EtOAc (2×50 mL). The organic layers were dried over Na₂SO₄, filtered and concentrated in vacuo. The residue was purified by silica gel column chromatography (SiO₂, petroleum ether:ethyl acetate, 5:1 to 3:1) to give the title compound (4 g, 62%) as a yellow solid.

¹H NMR (CDCl₃): δ 7.24-7.20 (m, 3H), 7.11 (dd, 4H), 7.00-6.95 (m, 2H), 6.80 (dd, 4H), 4.03 (s, 2H), 3.96 (s, 4H) and 3.74 (s, 6H).

### Step B: N,N-Bis(4-methoxybenzyl)-1-phenylethanesulfonamide

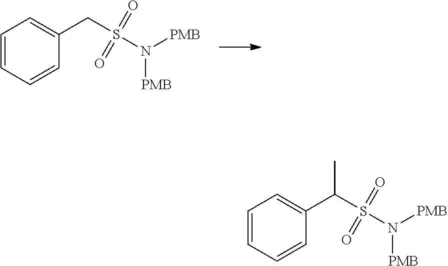

To a solution of N,N-bis(4-methoxybenzyl)-1-phenylmethanesulfonamide (1 g, 2.43 mmol, 1 eq) in THF (10 mL) was added LDA (2 M, 1.34 mL, 1.1 eq) at −78° C. under N₂ atmosphere. The mixture was stirred at −78° C. for 1 hour. Iodomethane (379 mg, 2.67 mmol, 1.1 eq) was added and the resulting mixture was stirred at 20° C. for 2 hours. The reaction mixture was quenched with saturated aqueous NH₄Cl solution (20 mL) and then concentrated in vacuo to remove THF. The mixture was treated with water (10 mL) and extracted with EtOAc (3×15 mL). The combined organic layers were dried over Na₂SO₄, filtered and concentrated in vacuo. The residue was purified by silica gel column chromatography (SiO₂, petroleum ether:ethyl acetate, 1:0 to 5:1) to give the title compound (0.9 g, 87%) as a white solid.

¹H NMR (CDCl₃): δ 7.33-7.28 (m, 3H), 7.14 (d, 4H), 7.10-7.08 (m, 2H), 6.86 (dd, 4H), 4.09 (d, 2H), 4.03-4.01 (m, 1H), 3.83 (s, 6H), 3.76 (d, 2H) and 1.79 (d, 3H).

### Step C: 1-Phenylethanesulfonamide

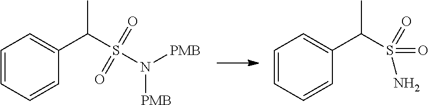

To a solution of N,N-bis(4-methoxybenzyl)-1-phenylethanesulfonamide (900 mg, 2.11 mmol, 1 eq) in DCM (30 mL) was added TFA (46.20 g, 405.19 mmol, 191.58 eq). The mixture was stirred at 20° C. for 12 hours. The reaction mixture was concentrated in vacuo. The residue was treated with MeOH (is mL). The suspension was filtered and the filtrate was concentrated in vacuo. The residue was triturated with a mixture of petroleum ether and ethyl acetate (v:v=20:1, 10 mL) to give the title compound (300 mg, 77%) as a white solid.

¹H NMR (CDCl₃): δ 7.47-7.39 (m, 5H), 4.46 (br s, 2H), 4.29 (q, 1H) and 1.82 (d, 3H).

### Intermediate P29: 1-Cyclopropyl-1H-pyrazole-3-sulfonamide

### Step A: 1-Cyclopropyl-3-nitro-1H-pyrazole

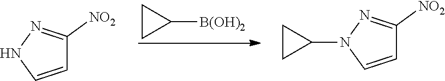 [SMILES: O=[N+]([O-])c1cc[nH]n1 ; OB(O)C1CC1 ; O=[N+]([O-])c1ccn(C2CC2)n1]

To a solution of cyclopropylboronic acid (36.77 g, 428.04 mmol, 1.1 eq) in DCE (500 mL) was added 3-nitro-1H-pyrazole (44 g, 389.12 mmol, 1 eq), 2,2-bipyridine (60.77 g, 389.12 mmol, 1 eq) and Na₂CO₃ (64.59 g, 609.44 mmol, 1.57 eq) at 25° C. The mixture was stirred at 25° C. for 0.5 hour. Then Cu(OAc)₂ (70.68 g, 389.12 mmol, 1 eq) was added and the resulting mixture was warmed to 70° C. and stirred at 70° C. for 15.5 hours. The reaction mixture was concentrated under reduced pressure to remove the solvent. The residue was purified by silica gel column chromatography (SiO₂, petroleum ether: ethyl acetate, 30:1 to 3:1) to give impure product (26.7 g). The impure product was dissolved in pyrrolidine (10 mL) and the resulting mixture was stirred at 70° C. for 2 hours. The reaction mixture was concentrated under reduced pressure to remove pyrrolidine. The residue was diluted with H₂O (33 mL) and the pH was adjusted to 5-6 with aqueous HCl solution (1N). Then the mixture was extracted with EtOAc (3×50 mL). The combined organic layers were washed with brine (2×33 mL), dried over Na₂SO₄, filtered and concentrated under reduced pressure to give the title compound (17.7 g, 30%) as yellow oil.

¹H NMR (CDCl₃): δ 7.54 (d, 1H), 6.84 (d, 1H), 3.73-3.67 (m, 1H), 1.24-1.22 (m, 2H) and 1.13-1.07 (m, 2H).

### Step B: 1-Cyclopropyl-1H-pyrazol-3-amine

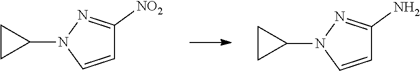 [SMILES: O=[N+]([O-])c1ccn(C2CC2)n1 ; Nc1ccn(C2CC2)n1]

To a solution of 1-cyclopropyl-3-nitro-1H-pyrazole (36 g, 235.08 mmol, 1 eq) in EtOH (400 mL) was added a solution of NH₄Cl (62.87 g, 1.18 mol, 5 eq) in H₂O (150 mL). Then the reaction mixture was warmed to 60° C. and iron power (39.38 g, 705.24 mmol, 3 eq) was added in portions. The reaction mixture was stirred at 60° C. for 16 hours and then concentrated under reduced pressure. The residue was diluted with H₂O (500 mL) and extracted with EtOAc (3×500 mL). The combined organic layers were washed with brine (2×250 mL), dried over Na₂SO₄, filtered and concentrated under reduced pressure. The residue was purified by silica gel column chromatography (SiO₂, petroleum ether:ethyl acetate, 30:1 to 1:1) to give the title compound (20 g, 69%) as yellow oil.

¹H NMR (CDCl₃): δ 7.14 (d, 1H), 5.11 (d, 1H), 3.57 (br s, 2H), 3.38-3.32 (m, 1H), 0.99-0.95 (m, 2H) and 0.90-0.87 (m, 2H).

LCMS: m/z 124.2 (M+H)⁺ (ES^{+).}

### Step C: 1-Cyclopropyl-1H-pyrazole-3-sulfonyl chloride

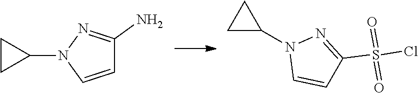 [SMILES: Nc1ccn(C2CC2)n1 ; O=S(=O)(Cl)c1ccn(C2CC2)n1]

To a solution of 1-cyclopropyl-1H-pyrazol-3-amine (19 g, 154.28 mmol, 1 eq) in MeCN (500 mL) and H₂O (50 mL) at 0° C. was added concentrated HCl solution (50 mL). Then an aqueous solution of NaNO₂ (12.77 g, 185.13 mmol, 1.2 eq) in H₂O (50 mL) was added slowly. The resulting solution was stirred at 0° C. for 40 minutes. AcOH (50 mL), CuCl₂ (10.37 g, 77.14 mmol, 0.5 eq) and CuCl (763 mg, 7.71 mmol, 0.05 eq) were added. Then SO₂ gas (15 psi) was bubbled into the resulting mixture for 20 minutes at 0° C. The reaction mixture was stirred at 0° C. for 1 hour and then concentrated under reduced pressure. The residue was diluted with H₂O (250 mL) and extracted with EtOAc (3×250 mL). The combined organic layers were washed with brine (2×150 mL), dried over Na₂SO₄, filtered and concentrated under reduced pressure. The residue was purified by silica gel column chromatography (SiO₂, petroleum ether:ethyl acetate, 100:0 to 1:1) to give the title compound (14 g, 44%) as yellow oil.

¹H NMR (CDCl₃): δ7.62 (d, 1H), 6.83 (d, 1H), 3.78-3.72 (m, 1H), 1.28-1.24 (m, 2H) and 1.16-1.12 (m, 2H).

### Step D: 1-Cyclopropyl-N,N-bis(4-methoxybenzyl)-1H-pyrazole-3-sulfonamide

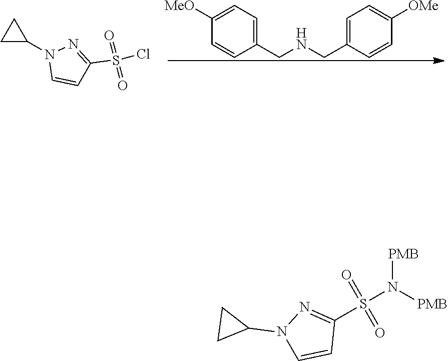

To a solution of 1-cyclopropyl-1H-pyrazole-3-sulfonyl chloride (28 g, 135.49 mmol, 1 eq) in THF (300 mL) was added TEA (27.42 g, 270.99 mmol, 2 eq) and bis(4-methoxybenzyl)amine (34.87 g, 135.49 mmol, 1 eq). The mixture was stirred at 25° C. for 1 hour. The reaction mixture was diluted with H₂O (500 mL) and extracted with EtOAc (3×500 mL). The combined organic layers were washed with brine (2×500 mL), dried over Na₂SO₄, filtered and concentrated under reduced pressure. The residue was purified by reversed phase flash chromatography (0.5% NH₃.H₂O-MeCN) to give the title compound (30 g, 52% yield, 99.8% purity on LCMS).

¹H NMR (CDCl₃): δ 7.49 (d, 1H), 7.08-7.06 (m, 4H), 6.79-6.77 (m, 4H), 6.62 (d, 1H), 4.32 (s, 4H), 3.80 (s, 6H), 3.68-3.64 (m, 1H), 1.15-1.13 (m, 2H) and 1.09-1.06 (m, 2H)

LCMS: m/z 428.2 (M+H)⁺ (ES⁺).

### Step E: 1-Cyclopropyl-1H-pyrazole-3-sulfonamide

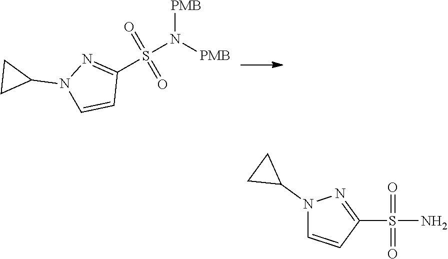

To a solution of 1-cyclopropyl-N,N-bis(4-methoxybenzyl)-1H-pyrazole-3-sulfonamide (1 g, 2.34 mmol, 1 eq) in DCM (10 mL) was added TFA (15.40 g, 135.06 mmol, 57.74 eq). The mixture was stirred at 25° C. for 12 hours. Most of the solvent was evaporated and the residue was re-dissolved in MeOH (30 mL). Solids were formed and the mixture was filtered. The filtrate was concentrated in vacuo and then the crude product was triturated with a mixture of PE and EtOAc (30 mL, 20:1) to give the title compound (430 mg, 88% yield, 90% purity on LCMS) as a white solid.

¹H NMR (DMSO-d₆): δ 7.92 (s, 1H), 7.38 (s, 2H), 6.55 (s, 1H), 3.84-3.78 (m, 1H) and 1.10-0.98 (m, 4H).

### Intermediate P30: 1-Cyclopropyl-1H-pyrazole-4-sulfonamide

### Step A: 4-Iodo-1-(tetrahydro-2H-pyran-2-yl)-1H-pyrazole

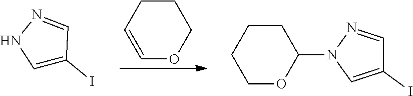 [SMILES: Ic1cn[nH]c1 ; C1=COCCC1 ; Ic1cnn(C2CCCCO2)c1]

To a mixture of 4-iodo-1H-pyrazole (50 g, 257.77 mmol, 1 eq) and pyridin-1-ium 4-methylbenzenesulfonate (32.39 g, 128.88 mmol, 0.5 eq) in DCM (500 mL) at 20° C. was added 3,4-dihydro-2H-pyran (43.4 g, 515.54 mmol, 2 eq). The reaction mixture was stirred at 20° C. for 12 hours and then concentrated in vacua. The residue was purified by silica gel column chromatography (SiO₂, petroleum ether:ethyl acetate, 1:0 to 20:1) to give the title compound (65 g, 91%) as a colourless oil.

¹H NMR (CDCl₃): δ 7.67 (s, 1H), 7.55 (s, 1H), 3.84-3.82 (m, 1H), 4.15-4.01 (m, 1H), 3.72-3.66 (m, 1H), 2.07-2.04 (m, 2H) and 1.69-1.62 (m, 4H).

### Step B: S-(1-(Tetrahydro-2H-pyran-2-yl)-1H-pyrazol-4-yl)benzothioate

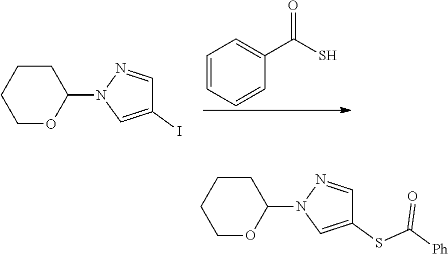 [SMILES: Ic1cnn(C2CCCCO2)c1 ; O=C(S)c1ccccc1 ; O=C(Sc1cnn(C2CCCCO2)c1)c1ccccc1]

CuI (2.05 g, 10.79 mmol, 0.1 eq) was added to the mixture of 4-iodo-1-(tetrahydro-2H-pyran-2-yl)-1H-pyrazole (30 g, 107.88 mmol, 1 eq), benzenecarbothioic S-acid (17.89 g, 129.45 mmol, 1.2 eq), 1,10-phenanthroline (3.89 g, 21.58 mmol, 0.2 eq) and DIPEA (27.89 g, 215.76 mmol, 2 eq) in toluene (300 mL) at 20° C. under N₂. The mixture was stirred for 12 hours at 110° C. under N₂. The residue was poured into 1 M HCl solution (500 mL). The aqueous phase was extracted with ethyl acetate (3×200 mL). The combined organic phases were washed with brine (200 mL), dried over anhydrous Na₂SO₄, filtered and concentrated in vacua. The residue was purified by silica gel column chromatography (SiO₂, petroleum ether:ethyl acetate, 20:1 to 5:1) to give the title compound (28 g, 85% yield, 94% purity on LCMS) as a yellow oil.

¹H NMR (CDCl₃): δ 8.01 (d, 2H), 7.83 (s, 1H), 7.64-7.59 (m, 2H), 7.49 (t, 2H), 5.49 (t, 1H), 4.09-4.05 (m, 1H), 3.76-3.69 (m, 1H), 2.16-2.13 (m, 2H), 1.74-1.62 (m, 4H).

### Step C: 1-(Tetrahydro-2H-pyran-2-yl)-1H-pyrazole-4-sulfonyl chloride

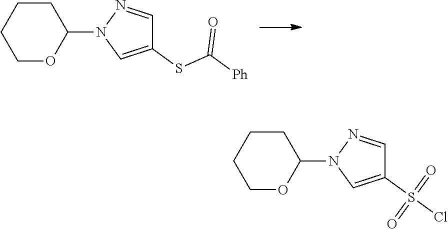 [SMILES: O=C(Sc1cnn(C2CCCCO2)c1)c1ccccc1 ; O=S(=O)(Cl)c1cnn(C2CCCCO2)c1]

1,3,5-Trichloro-1,3,5-triazinane-2,4,6-trione (13.30 g, 57.22 mmol, 1.1 eq) was added into a solution of benzyltrimethylammonium chloride (31.88 g, 171.66 mmol, 29.79 mL, 3.3 eq) in MeCN (300 mL) at 20° C. The mixture was stirred for 30 minutes. The clear yellow solution was added dropwise into a solution of S-(1-(tetrahydro-2H-pyran-2-yl)-1H-pyrazol-4-yl)benzothioate (15 g, 52.02 mmol, 1 eq) in MeCN (150 mL) at 0° C. An aqueous sodium carbonate solution (1 M, 52.02 mL, 1 eq) was added dropwise into the mixture at 0° C. The mixture was stirred for 30 minutes. The reaction solution was diluted with saturated aqueous sodium carbonate solution (100 mL) and extracted with EtOAc (2×100 mL). The combined organic layers were concentrated in vacuo. The residue was purified by silica gel column chromatography (SiO₂, petroleum ether:ethyl acetate, 20:1 to 5:1) to give the title compound (3.5 g, 27%) as a colourless oil.

¹H NMR (CDCl₃): δ 8.29 (s, 1H), 8.00 (s, 1H), 5.45 (q, 1H), 4.16-4.08 (m, 1H), 3.78-3.74 (m, 1H), 2.02-1.96 (m, 2H) and 1.71-1.60 (m, 4H).

### Step D: N,N-Bis(4-methoxybenzyl)-1-(tetrahydro-2H-pyran-2-yl)-1H-pyrazole-4-sulfonamide

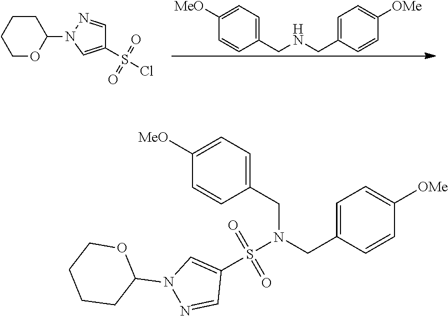 [SMILES: O=S(=O)(Cl)c1cnn(C2CCCCO2)c1 ; COc1ccc(CNCc2ccc(OC)cc2)cc1 ; COc1ccc(CN(Cc2ccc(OC)cc2)S(=O)(=O)c2cnn(C3CCCCO3)c2)cc1]

1-(Tetrahydro-2H-pyran-2-yl)-1H-pyrazole-4-sulfonyl chloride (2.5 g, 9.97 mmol, 1 eq) was added into the solution of bis(4-methoxybenzyl)amine (2.31 g, 8.97 mmol, 0.9 eq) and TEA (3.03 g, 29.92 mmol, 3 eq) in THF (50 mL) at 0° C. The reaction mixture was stirred at 20° C. for 12 hours. The residue was poured into 1 M HCl solution (100 mL). The aqueous phase was extracted with ethyl acetate (2×30 mL). The combined organic phases were washed with brine (20 mL), dried over anhydrous Na₂SO₄, filtered and concentrated in vacuo. The solid was triturated with a mixture of PE and EtOAc (20 mL, v:v=5:1) to give the title compound (3 g, 60% yield, 94.4% purity on LCMS) as a white solid.

¹H NMR (CDCl₃): δ 7.76 (s, 1H), 7.65 (s, 1H), 7.11 (d, 4H), 6.81 (d, 4H), 3.35 (q, 1H), 4.23 (s, 4H), 4.05 (d, 1H), 3.80 (s, 6H), 3.73-3.64 (m, 1H), 2.10-1.97 (m, 2H) and 1.76-1.64 (m, 4H).

LCMS: m/z 472.1 (M+H)⁺ (ES⁺).

### Step E: N,N-Bis(4-methoxybenzyl)-1H-pyrazole-4-sulfonamide

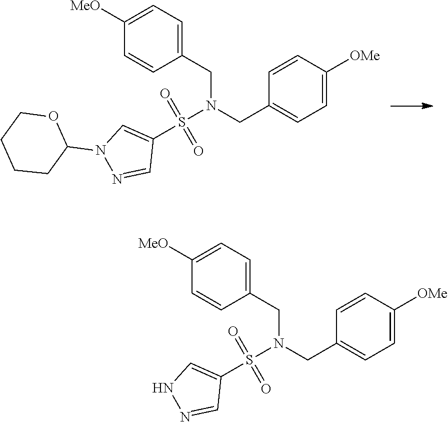 [SMILES: COc1ccc(CN(Cc2ccc(OC)cc2)S(=O)(=O)c2cnn(C3CCCCO3)c2)cc1 ; COc1ccc(CN(Cc2ccc(OC)cc2)S(=O)(=O)c2cn[nH]c2)cc1]

HCl (1 M, 8.48 mL, 2 eq) was added to the mixture of N,N-bis(4-methoxybenzyl)-1-(tetrahydro-2H-pyran-2-yl)-1H-pyrazole-4-sulfonamide (2 g, 4.24 mmol, 1 eq) in EtOH (20 mL) and THF (20 mL) at 20° C. The mixture was stirred at 20° C. for 12 hours. The reaction mixture was poured into saturated aqueous sodium bicarbonate solution (30 mL). The aqueous phase was extracted with ethyl acetate (3×20 mL). The combined organic phases were washed with brine (20 mL), dried over anhydrous Na₂SO₄, filtered and concentrated in vacua to give the title compound (2 g, crude) as a yellow oil, which was used in the next step without further purification.

¹H NMR (CDCl₃): δ 7.78 (s, 2H), 7.10 (d, 4H), 6.81 (d, 4H), 4.24 (s, 4H) and 3.79 (s, 6H).

LCMS: m/z 388.1 (M+H)⁺ (ES⁺).

### Step F: 1-Cyclopropyl-N,N-bis(4-methoxybenzyl)-1H-pyrazole-4-sulfonamide

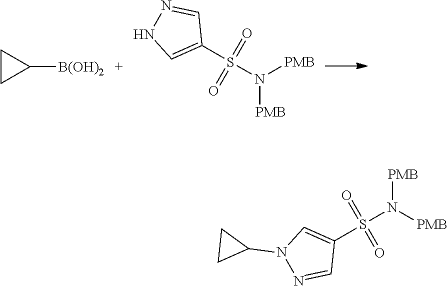

To a solution of cyclopropylboronic acid (109 mg, 1.28 mmol, 1.1 eq) in dioxane (5 mL) was added N,N-bis(4-methoxybenzyl)-1H-pyrazole-4-sulfonamide (450 mg, 1.16 mmol, 1 eq), 2,2-bipyridine (181.39 mg, 1.16 mmol, 1 eq) and Na₂CO₃ (193 mg, 1.82 mmol, 1.57 eq). The reaction mixture was stirred at 25° C. for 0.5 hour. Then Cu(OAc)₂ (211 mg, 1.16 mmol, 1 eq) was added and the resulting mixture was warmed to 70° C. and stirred at 70° C. for 11.5 hours. The reaction mixture was diluted with H₂O (20 mL) and extracted with EtOAc (3×50 mL). The combined organic layers were washed with brine (2×20 mL), dried over Na₂SO₄, filtered and concentrated under reduced pressure to give a residue. The residue was purified by silica gel column chromatography (SiO₂, petroleum ether:ethyl acetate, 20:1 to 1:1) to give the title compound (210 mg, 42%) as a yellow solid.

¹H NMR (DMSO-d₆): δ 8.31 (s, 1H), 7.78 (s, 1H), 7.09-7.05 (m, 4H), 6.83-6.80 (m, 4H), 4.14 (s, 4H), 3.83-3.77 (m, 1H), 3.72 (s, 6H), 1.08-1.03 (m, 2H) and 1.02-1.00 (m, 2H).

LCMS: m/z 428.2 (M+H)⁺ (ES⁺)

### Step G: 1-Cyclopropyl-1H-pyrazole-4-sulfonamide

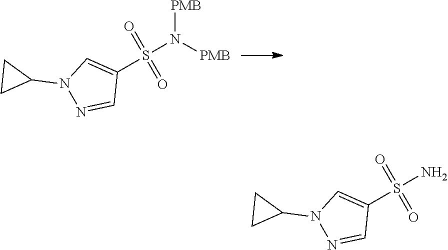

To a solution of 1-cyclopropyl-N,N-bis(4-methoxybenzyl)-1H-pyrazole-4-sulfonamide (170 mg, 397.65 μmol, 1 eq) in DCM (1 mL) was added TFA (5.24 g, 45.92 mmol, 115.48 eq). The mixture was stirred at 25° C. for 2 hours. Most of the solvent was evaporated to give the crude product. The crude product was added into MeOH (3 mL) and solid was formed. The mixture was filtered and the filtrate was concentrated in vacuo to give the title compound (44 mg, 59%) as a red solid.

¹H NMR (DMSO-d₆): δ 8.29 (s, 1H), 7.74 (s, 1H), 7.23 (s, 2H), 3.3-3.79 (m, 1H), 1.08-1.05 (m, 2H) and 1.01-0.98 (m, 2H).

LCMS: m/z 188.1 (M+H)⁺ (ES⁺).

### Intermediate P31: (1-Methylpyrrolidin-3-yl)methanesulfonamide

### Step A: tert-Butyl 3-(((methylsulfonyl)oxy)methyl)pyrrolidine-1-carboxylate

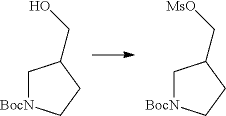 [SMILES: CC(C)(C)OC(=O)N1CCC(CO)C1 ; CC(C)(C)OC(=O)N1CCC(COS(C)(=O)=O)C1]

To a mixture of tert-butyl 3-(hydroxymethyl)pyrrolidine-1-carboxylate (13 g, 64.59 mmol, 1 eq) and TEA (13.07 g, 129.18 mmol, 2.0 eq) in DCM (200 mL) was added dropwise MSCl (8.23 g, 71.85 mmol, 1.1 eq) at 0° C. Then the reaction mixture was warmed to 25° C. and stirred for 1 hour under N₂. The reaction mixture was quenched with water (100 mL) and extracted with DCM (3×100 mL). The combined organic phases were washed with brine (100 mL), dried over anhydrous Na₂SO₄, filtered and concentrated in vacuo to give the title compound (20 g, crude) as brown oil, which was used directly in the next step without further purification.

### Step B: tert-Butyl 3-((acetylthio)methyl)pyrrolidine-1-carboxylate

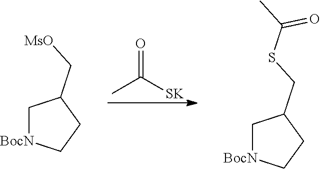

To a mixture of tert-butyl 3-(((methylsulfonyl)oxy)methyl)pyrrolidine-1-carboxylate (20 g, 71.59 mmol, 1 eq) in acetonitrile (300 mL) was added potassium ethanethioate (10 g, 87.56 mmol, 1.22 eq) in one portion. Then the reaction mixture was heated to 50° C. and stirred for 12 hours. The mixture was concentrated in vacua. The residue was treated with water (100 mL) and the mixture was extracted with EtOAc (3×100 mL). The combined organic phases were washed with brine (100 mL), dried over anhydrous Na₂SO₄, filtered and concentrated in vacua. The residue was purified by silica gel chromatography (SiO₂, petroleum ether:ethyl acetate, 50:1 to 5:1) to give the title compound (14.2 g, 76%) as a yellow oil.

¹H NMR (CDCl₃): δ 3.61-3.41 (m, 2H), 3.33-3.23 (m, 1H), 3.05-2.87 (m, 3H), 2.42-2.29 (m, 4H), 2.08-1.99 (m, 1H), 1.64-1.59 (m, 1H) and 1.46 (s, 9H).

### Step C: tert-Butyl 3-((chlorosulfonyl)methyl)pyrrolidine-1-carboxylate

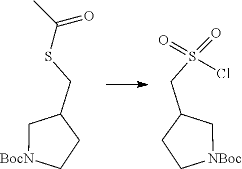 [SMILES: CC(=O)SCC1CCN(C(=O)OC(C)(C)C)C1 ; CC(C)(C)OC(=O)N1CCC(CS(=O)(=O)Cl)C1]

To a mixture of tert-butyl 3-((acetylthio)methyl)pyrrolidine-1-carboxylate (4 g, 15.42 mmol, 1 eq) in AcOH (200 mL) and H₂O (20 mL) was added NCS (6.18 g, 46.27 mmol, 3 eq) in one portion at 25° C. Then the reaction mixture was stirred at 25° C. for 1 hour. The mixture was quenched with water (200 mL) and extracted with DCM (2×100 mL). The combined organic phases were washed with brine (2×100 mL), dried over anhydrous Na₂SO₄, filtered and concentrated in vacua to give a solution of the title compound (4.38 g, crude) in DCM (200 mL), which was used directly in the next step without further purification.

### Step D: tert-Butyl 3-(sulfamoylmethyl)pyrrolidine-1-carboxylate

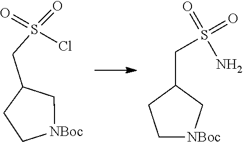 [SMILES: CC(C)(C)OC(=O)N1CCC(CS(=O)(=O)Cl)C1 ; CC(C)(C)OC(=O)N1CCC(CS(N)(=O)=O)C1]

NH₃ (15 psi) was bubbled into a solution of tert-butyl 3-((chlorosulfonyl)methyl)pyrrolidine-1-carboxylate (4.38 g, crude) in DCM (200 mL) at −20° C. for 10 minutes. Then the reaction mixture was filtered and the filtrate was concentrated in vacuo to give the title compound (2 g, crude) as a brown solid.

¹H NMR (CDCl₃): δ 3.78-3.73 (m, 1H), 3.56-3.47 (m, 1H), 3.37-3.31 (m, 1H), 3.25-3.15 (m, 2H), 3.14-3.04 (m, 1H), 2.78-2.72 (m, 1H), 2.26-2.20 (m, 1H), 1.77-1.71 (m, 1H) and 1.47 (s, 9H).

### Step E: Pyrrolidin-3-ylmethanesulfonamide hydrochloride

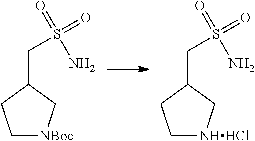

To a mixture of tert-butyl 3-(sulfamoylmethyl)pyrrolidine-1-carboxylate (2 g, 7.57 mmol, 1 eq) in EtOAc (5 mL) was added a solution of HCl in EtOAc (4 M, 30 mL, 15.86 eq) in one portion. Then the reaction mixture was stirred at 25° C. for 0.5 hour. The reaction mixture was concentrated in vacua to give the title compound (2 g, crude, HCl salt) as a brown oil, which was used directly in the next step without further purification.

¹H NMR (DMSO-d₆): δ 9.35-9.23 (m, 2H), 6.99 (s, 2H), 3.39-3.36 (m, 1H), 3.22-3.19 (m, 2H), 3.08-3.05 (m, 1H), 2.93-2.85 (m, 1H), 2.65-2.59 (m, 2H), 2.20-2.13 (m, 1H) and 1.71-1.63 (m, 1H).

### Step F: (1-Methylpyrrolidin-3-yl)methanesulfonamide

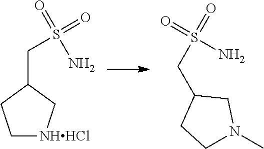

To a solution of pyrrolidin-3-ylmethanesulfonamide hydrochloride (2 g, 9.97 mmol, 1 eq), TEA (1.21 g, 11.96 mmol, 1.2 eq) and HCHO (849 mg, 10.46 mmol, 1.05 eq) in MeCN (20 mL) was added NaBH(OAc)₃ (2.64 g, 12.46 mmol, 1.25 eq) in one portion. Then the reaction mixture was stirred at 25° C. for 12 hours. The mixture was concentrated in vacuo. The residue was purified by reversed phase flash (0.05% NH₃.H₂O in water/MeCN) and then further purified by silica gel chromatography (0.1% NH₃.H₂O, EtOAc: EtOH, 1:0 to 1:1) to give the title compound (1.5 g, 84%) as a yellow solid.

¹H NMR (DMSO-d₆): δ 5.60 (br s, 2H), 3.04-3.01 (m, 2H), 2.70-2.65 (m, 1H), 2.45-2.37 (m, 2H), 2.30-2.21 (m, 5H), 2.08-1.95 (m, 1H) and 1.56-1.50 (m, 1H).

LCMS: m/z 179.1 (M+H)⁺ (ES⁺).

### Intermediate P32: 3-(Diethylamino)propane-1-sulfonamide

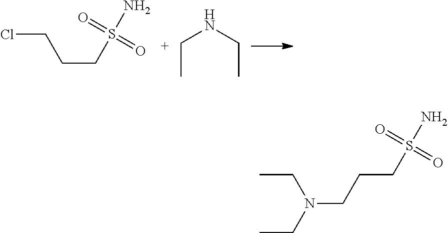

To a solution of 3-chloropropane-1-sulfonamide (203 mg, 1.29 mmol) in acetonitrile (10 mL) was added triethylamine (214 μL, 1.55 mmol, 1.2 equiv.), N,N-diethylamine (159 μL, 1.55 mmol, 1.2 equiv.) and potassium iodide (43 mg, 0.26 mmol) and the reaction mixture was irradiated in the microwave at 100° C. for 90 minutes. Additional potassium iodide (150 mg) was added and the resulting mixture was heated conventionally for another 2 hours at 100° C. Upon cooling to room temperature the mixture was concentrated in vacua to afford the crude title compound (>100% yield); the material still contained salts and impurities but was used without further purification.

¹H NMR (CD₃OD) δ 2.86 (m, 6H), 2.47 (m, 2H), 2.23 (m, 2H) and 1.18 (t, 6H).

LCMS: m/z 195.1 (M+H)⁺ (ES⁺).

### Intermediate P33: 3-(Benzyl(ethyl)amino)propane-1-sulfonamide

### Step A: 3-(Benzyl(ethyl)amino)propane-1-sulfonic acid

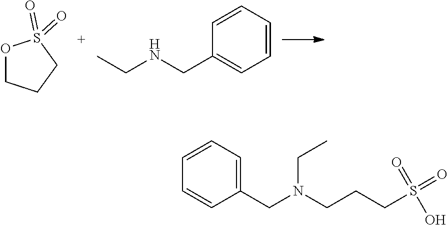

To a solution of 1,2-oxathiolane 2,2-dioxide (1 g, 8.19 mmol, 719.42 μL,1 eq) in DCM (5 mL) was added N-benzylethanamine (3.94 g, 29.15 mmol, 3.56 eq) at 0° C. Then the resulting mixture was stirred at 25° C. for 2.5 hours. The mixture was concentrated in vacua. The residue was triturated with EtOAc (40 mL) to give the title compound (2.4 g, crude) as a white solid.

¹H NMR (DMSO-d₆): δ 7.37-7.23 (m, 5H), 4.08 (s, 2H), 2.91 (q, 2H), 2.50-2.40 (m, 4H), 1.81-1.73 (m, 2H) and 0.98 (t, 3H).

LCMS: m/z 258.1 (M+H)⁺ (ES⁺).

### Step B: 3-(Benzyl(ethyl)amino)propane-1-sulfonyl chloride

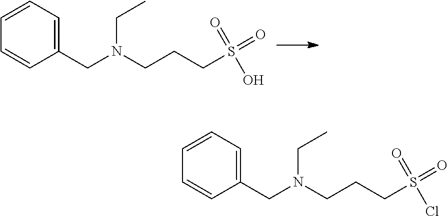 [SMILES: CCN(CCCS(=O)(=O)O)Cc1ccccc1 ; CCN(CCCS(=O)(=O)Cl)Cc1ccccc1]

A solution of 3-(benzyl(ethyl)amino)propane-1-sulfonic acid (2.1 g, 8.16 mmol, 1 eq) in SOCl₂ (17.22 g, 144.74 mmol, 17.74 eq) was stirred at 80° C. for 6 hours. The mixture was concentrated in vacua to give the title compound (2 g, crude) as a yellow oil, which was used directly in the next step.

### Step C: 3-(Benzyl(ethyl)amino)propane-1-sulfonamide

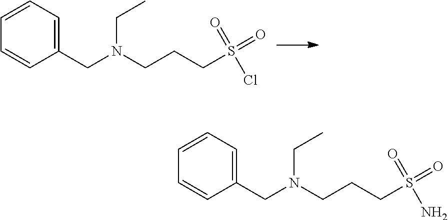 [SMILES: CCN(CCCS(=O)(=O)Cl)Cc1ccccc1 ; CCN(CCCS(N)(=O)=O)Cc1ccccc1]

To a solution of 3-(benzyl(ethyl)amino)propane-1-sulfonyl chloride (2 g, crude) in THF (3 mL) was added to a saturated solution of NH₃ in THF (100 mL) at 0° C. Then the mixture was stirred at 20° C. for 14 hours. The mixture was filtered and the filtrate was concentrated in vacuo. The residue was purified by reversed phase flash chromatography (0.1% NH₃.H₂O-MeCN) to give the title compound (1.15 g, 62% yield, 100% purity on LCMS) as a white solid.

¹H NMR (CDCl₃): δ 7.37-7.28 (m, 5H), 4.98 (br s, 2H), 3.57 (s, 2H), 3.15 (t, 2H), 2.61-2.52 (m, 4H), 2.06-2.00 (m, 2H) and 1.07 (t, 3H).

### Intermediate P34: 3-Methoxypropane-1-sulfonamide

### Step A: Sodium 3-methoxypropane-1-sulfonate

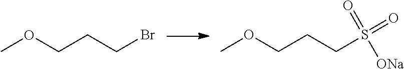

A mixture of 1-bromo-3-methoxypropane (2 g, 13.07 mmol, 1 eq) and Na₂SO₃ (1.65 g, 13.07 mmol, 1 eq) in H₂O (20 mL) was heated to 100° C. and stirred for 16 hours. Then the reaction mixture was cooled and lyophilized to give the title compound (2.25 g, 97% yield, Na salt) as a white solid.

¹H NMR (D₂O): δ 3.56 (t, 2H), 3.34 (s, 3H), 2.95-2.92 (m, 2H) and 2.02-1.94 (m, 2H).

LCMS: m/z 155.1 (M-Na+H)⁺ (ES^{+).}

### Step B: 3-Methoxypropane-1-sulfonyl chloride

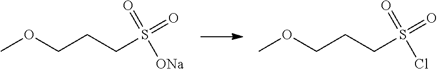

A solution of sodium 3-methoxypropane-1-sylfonate (0.7 g, 4.54 mmol, 1 eq) in POCl₃ (8.25 g, 53.80 mmol, 11.85 eq) was stirred at 80° C. for 5 hours. Then the mixture was stirred at 100° C. for 2 hours. The mixture was diluted with DCM (80 mL) and filtered. The filtrate was concentrated in vacuo to give the title compound (600 mg, crude) as a yellow oil, which was used directly in the next step.

### Step C: 3-Methoxypropane-1-sulfonamide

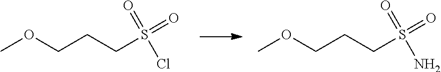

NH₃ (15 psi) was bubbled into THF (20 mL) at 0° C. for 5 minutes. A solution of 3-methoxypropane-1-sulfonyl chloride (600 mg, crude) in THF (2 mL) was added to the NH₃/THF solution (20 mL). Then the mixture was stirred at 20° C. for 14 hours. The reaction mixture was filtered and the filtrate was concentrated in vacua to give the crude compound (300 mg, crude) as a yellow oil.

¹H NMR (CDCl₃): δ 4.94 (br s, 2H), 3.53 (t, 2H), 3.35 (s, 3H), 3.25 (t, 2H) and 2.17-2.10 (m, 2H).

### Intermediate P35: N,N-Bis(2-methoxyethyl)-1-methyl-3-sulfamoyl-1H-pyrazole-5-carboxamide

### Step A: 1-Methyl-1H-pyrazole-3-sulfonyl chloride

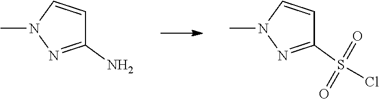

A solution of 1-methyl-1H-pyrazol-3-amine (25 g, 257.42 mmol, 1 eq) in MeCN (boo mL) at 0° C. was treated with concentrated HCl (60 mL) and H₂O (60 mL). Then an aqueous solution of NaNO₂ (21.31 g, 308.90 mmol, 1.2 eq) in H₂O (60 mL) was added slowly. The resulting mixture was stirred at 0° C. for 40 minutes. AcOH (60 mL), CuCl₂ (17.31 g, 128.71 mmol, 0.5 eq) and CuCl (1.27 g, 12.87 mmol, 307.78 μL, 0.05 eq) were added, then SO₂ gas (15 psi) was bubbled into the mixture for 15 minutes at 0° C. The reaction mixture was concentrated in vacuo to remove most of the MeCN. Then the reaction mixture was treated with H₂O (2.5 L) and extracted with EtOAc (2×1.2 L). The combined organic layers were washed with brine (3×2 L), dried over anhydrous Na₂SO₄, filtered and concentrated under reduced pressure. The residue was purified by silica gel column chromatography (petroleum ether:ethyl acetate=15:1 to 5:1) to give the title compound (19 g, 41%) as a yellow oil.

¹H NMR (CDCl₃): δ 7.52 (d, 1H), 6.89 (d, 1H) and 4.07 (s, 3H).

### Step B: N,N-Bis(4-methoxybenzyl)-1-methyl-1H-pyrazole-3-sulfonamide

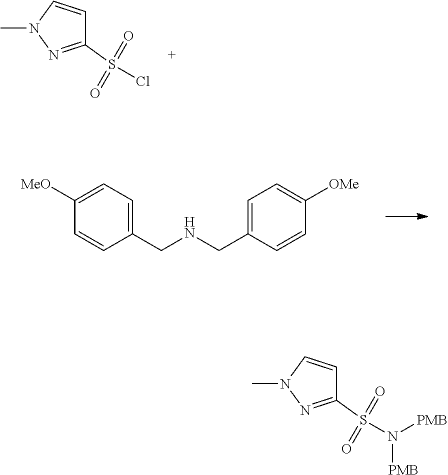

To a solution of bis(4-methoxybenzyl)amine (99.83 g, 387.96 mmol, 0.91 eq) in THF (1 L) was added TEA (86.28 g, 852.65 mmol, 118.68 mL, 2 eq), followed by 1-methyl-1H-pyrazole-3-sulfonyl chloride (77 g, 426.33 mmol, 1 eq). Then the reaction mixture was stirred at 25° C. for 12 hours. The reaction mixture was concentrated in vacua to remove most of the THF. The reaction mixture was quenched by addition of aqueous HCl (1 M, 500 mL) and then extracted with EtOAc (2×500 mL). The combined organic layers were washed with brine (2×600 mL), dried over anhydrous Na₂SO₄, filtered and concentrated under reduced pressure. The residue was triturated with a mixture of petroleum ether and ethyl acetate (70 mL, v:v=5:1) to give the title compound (138 g, 81%) as a white solid.

¹H NMR (CDCl₃): δ 7.40 (d, 1H), 7.08 (d, 4H), 6.78 (d, 4H), 6.65-6.63 (m, 1H), 4.32 (s, 4H), 3.98 (s, 3H) and 3.79 (s, 6H).

LCMS: m/z 402.2 (M+H)⁺ (ES⁺).

### Step C: 3-(N,N-Bis(4-methoxybenzyl)sulfamoyl)-1-methyl-1H-pyrazole-5-carboxylic acid

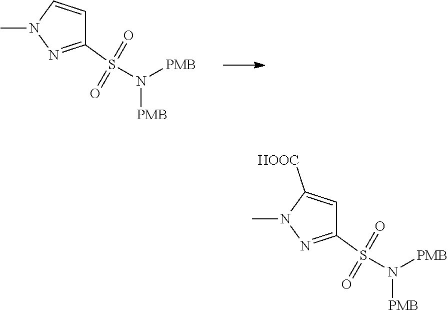

A solution of N,N-bis(4-methoxybenzyl)-1-methyl-1H-pyrazole-3-sulfonamide (100 g, 249.08 mmol, 1 eq) in THF (1.35 L) was cooled to −70° C. Then n-BuLi (2.5 M, 104.61 mL, 1.05 eq) was added dropwise. The reaction mixture was stirred at −70° C. for 1 hour, then CO₂ (15 psi) was bubbled into the mixture for 15 minutes. The reaction mixture was stirred at −70° C. for another 1 hour. The reaction mixture was quenched with H₂O (1.2 L) and adjusted with aqueous HCl (1 M) to pH=3. Then the mixture was extracted with EtOAc (2×1 L). The combined organic layers were washed with brine (2×1 L), dried over Na₂SO₄, filtered and concentrated in vacua. The residue was triturated with a mixture of petroleum ether and ethyl acetate (300 mL, v:v=1:1) to give the title compound (94 g, 84% yield, 99% purity on LCMS) as a white solid.

¹H NMR (DMSO-d₆): δ 6.98-7.16 (m, 5H), 6.82 (d, 4H), 4.25 (s, 4H), 4.15 (s, 3H) and 3.72 (s, 6H).

LCMS: m/z 468.2 (M+Na)+ (ES+).

### Step D: 3-(N,N-Bis(4-methoxybenzyl)sulfamoyl)-N,N-bis(2-methoxyethyl)-1-methyl-1H-pyrazole-5-carboxamide

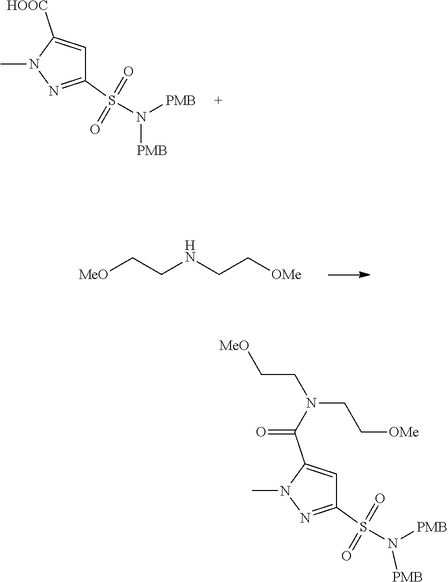

To a solution of 3-(N,N-bis(4-methoxybenzyl)sulfamoyl)-1-methyl-1H-pyrazole-5-carboxylic acid (8 g, 17.96 mmol, 1 eq) in DMF (100 mL) was added with HATU (10.24 g, 26.94 mmol, 1.5 eq), DIPEA (6.96 g, 53.87 mmol, 3 eq) and bis(2-methoxyethyl)amine (2.87 g, 21.55 mmol, 1.2 eq). The reaction mixture was stirred at 25° C. for 1 hour. Then the reaction mixture was diluted with EtOAc (50 mL), washed with saturated aqueous NH₄Cl solution (3×50 mL) and brine (3×50 mL). The organic layer was dried over anhydrous Na₂SO₄, filtered and concentrated in vacua. The residue was purified by reversed phase flash chromatography (0.05% NH₃.H₂O-MeCN) to give the title compound (8 g, 79%) as a red oil.

¹H NMR (CD₃OD): δ 7.05 (d, 4H), 6.81-6.77 (m, 5H), 4.29 (s, 4H), 3.90 (s, 3H), 3.79-3.72 (m, 8H), 3.68-3.57 (m, 4H), 3.48-3.46 (m, 2H), 3.38 (s, 3H) and 3.27 (s, 3H).

LCMS: m/z 561.3 (M+H)⁺ (ES⁺).

### Step E: N,N-Bis(2-methoxyethyl)-1-methyl-3-sulfamoyl-1H-pyrazole-5-carboxamide

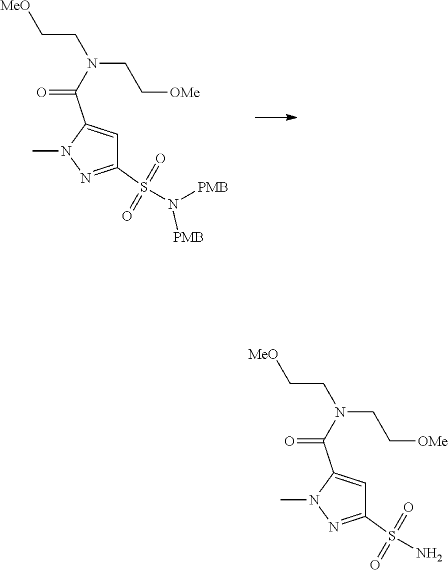

To a solution of 3-(N,N-bis(4-methoxybenzyl)sulfamoyl)-N,N-bis(2-methoxyethyl)-1-methyl-1H-pyrazole-5-carboxamide (8 g, 14.27 mmol, 1 eq) in DCM (50 mL) was added TFA (56 g, 491.13 mmol, 34.42 eq). The reaction mixture was stirred at 25° C. for 12 hours and then concentrated in vacuo. The residue was triturated with a mixture of EtOAc and PE (50 mL, v:v=3:2) to give the title compound (4.0 g, 88%) as a white solid.

¹H NMR (DMSO-d₆): δ 7.50 (s, 2H), 6.74 (s, 1H), 3.84 (s, 3H), 3.63 (t, 4H), 3.43-3.40 (m, 4H), 3.28 (s, 3H) and 3.18 (s, 3H).

### Intermediate P36: N,N,1-Trimethyl-3-sulfamoyl-1H-pyrazole-5-carboxamide

### Step A: 1-Methyl-1H-pyrazole-3-sulfonyl chloride

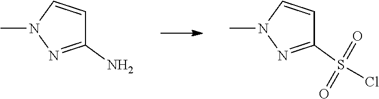

A solution of 1-methyl-1H-pyrazol-3-amine (25 g, 257.42 mmol, 1 eq) in MeCN (boo mL) at 0° C. was treated with concentrated HCl (60 mL) and H₂O (60 mL). Then an aqueous solution of NaNO₂ (21.31 g, 308.90 mmol, 1.2 eq) in H₂O (60 mL) was added slowly. The resulting mixture was stirred at 0° C. for 40 minutes. AcOH (60 mL), CuCl₂ (17.31 g, 128.71 mmol, 0.5 eq) and CuCl (1.27 g, 12.87 mmol, 307.78 μL, 0.05 eq) were added, then SO₂ gas (15 psi) was bubbled into the mixture for 15 minutes at 0° C. The reaction mixture was concentrated in vacuo to remove most of the MeCN. Then the reaction mixture was treated with H₂O (2.5 L) and extracted with EtOAc (2×1.2 L). The combined organic layers were washed with brine (3×2 L), dried over anhydrous Na₂SO₄, filtered and concentrated under reduced pressure. The residue was purified by silica gel column chromatography (petroleum ether:ethyl acetate=15:1 to 5:1) to give the title compound (19 g, 41%) as a yellow oil.

¹H NMR (CDCl₃): δ 7.52 (d, 1H), 6.89 (d, 1H) and 4.07 (s, 3H).

### Step B: N,N-Bis(4-methoxybenzyl)-1-methyl-1H-pyrazole-3-sulfonamide

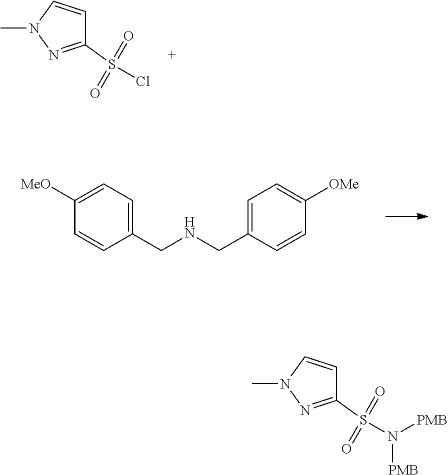

To a solution of bis(4-methoxybenzyl)amine (99.83 g, 387.96 mmol, 0.91 eq) in THF (1 L) was added TEA (86.28 g, 852.65 mmol, 118.68 mL, 2 eq), followed by 1-methyl-1H-pyrazole-3-sulfonyl chloride (77 g, 426.33 mmol, 1 eq). Then the reaction mixture was stirred at 25° C. for 12 hours. The reaction mixture was concentrated in vacua to remove most of the THF. The reaction mixture was quenched by addition of aqueous HCl (1 M, 500 mL) and then extracted with EtOAc (2×500 mL). The combined organic layers were washed with brine (2×600 mL), dried over anhydrous Na₂SO₄, filtered and concentrated under reduced pressure. The residue was triturated with a mixture of petroleum ether and ethyl acetate (70 mL, v:v=5:1) to give the title compound (138 g, 81%) as a white solid.

¹H NMR (CDCl₃): δ 7.40 (d, 1H), 7.08 (d, 4H), 6.78 (d, 4H), 6.65-6.63 (m, 1H), 4.32 (s, 4H), 3.98 (s, 3H) and 3.79 (s, 6H).

LCMS: m/z 402.2 (M+H)⁺ (ES⁺).

### Step C: 3-(N,N-Bis(4-methoxybenzyl)sulfamoyl)-1-methyl-1H-pyrazole-5-carboxylic acid

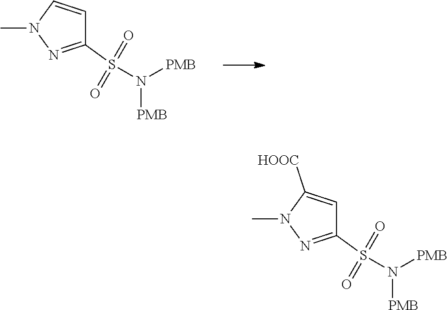

A solution of N,N-bis(4-methoxybenzyl)-1-methyl-1H-pyrazole-3-sulfonamide (100 g, 249.08 mmol, 1 eq) in THF (1.35 L) was cooled to −70° C. Then n-BuLi (2.5 M, 104.61 mL, 1.05 eq) was added dropwise. The reaction mixture was stirred at −70° C. for 1 hour, then CO₂ (15 psi) was bubbled into the mixture for 15 minutes. The reaction mixture was stirred at −70° C. for another 1 hour. The reaction mixture was quenched with H₂O (1.2 L) and adjusted with aqueous HCl (1 M) to pH=3. Then the mixture was extracted with EtOAc (2×1 L). The combined organic layers were washed with brine (2×1 L), dried over Na₂SO₄, filtered and concentrated in vacua. The residue was triturated with a mixture of petroleum ether and ethyl acetate (300 mL, v:v=1:1) to give the title compound (94 g, 84% yield, 99% purity on LCMS) as a white solid.

¹H NMR (DMSO-d₆): δ 6.98-7.16 (m, 5H), 6.82 (d, 4H), 4.25 (s, 4H), 4.15 (s, 3H) and 3.72 (s, 6H).

LCMS: m/z 468.2 (M+Na)+ (ES+).

### Step D: 3-(N,N-Bis(4-methoxybenzyl)sulfamoyl)-N,N,1-trimethyl-1H-pyrazole-5-carboxamide

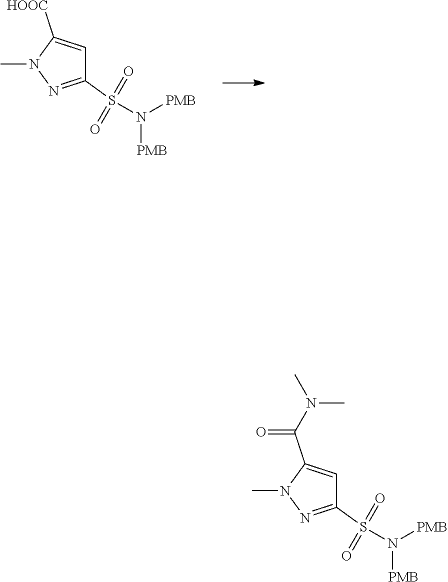

To a solution of 3-(N,N-bis(4-methoxybenzyl)sulfamoyl)-1-methyl-1H-pyrazole-5-carboxylic acid (100 g, 224.47 mmol, 1 eq), DIPEA (58.02 g, 448.95 mmol, 78.20 mL, 2 eq) and dimethylamine (2 M, 448.95 mL, 4 eq) in DMF (1 L) was added a solution of propylphosphonic anhydride in EtOAc (285.69 g, 448.95 mmol, 267.00 mL, 50% in EtOAc, 2 eq) at 25° C. Then the reaction mixture was stirred for 30 minutes. The reaction mixture was quenched by addition of H₂O (2 L) and then extracted with EtOAc (2×1.1 L). The combined organic layers were washed with brine (2×1.2 L), dried over anhydrous Na₂SO₄, filtered and concentrated under reduced pressure. The residue was triturated with a mixture of EtOAc and petroleum ether (v:v=5:1, 150 mL) to give the title compound (92.7 g, 87% yield, 100% purity on LCMS).

¹H NMR (CDCl₃): δ 7.09 (d, 4H), 6.78 (d, 4H), 6.63-6.70 (m, 1H), 4.32 (s, 4H), 4.02 (s, 3H), 3.79 (s, 6H) and 3.11 (d, 6H).

LCMS: m/z 473.3 (M+H)⁺ (ES⁺).

### Step E: N,N,1-Trimethyl-3-sulfamoyl-1H-pyrazole-5-carboxamide

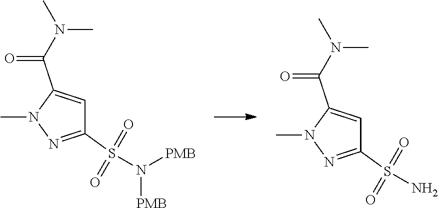

To a solution of 3-(N,N-bis(4-methoxybenzyl)sulfamoyl)-N,N,1-trimethyl-1H-pyrazole-5-carboxamide (80 g, 169.29 mmol, 1 eq) in DCM (180 mL) was added TFA (381.33 g, 3.34 mol, 247.62 mL, 19.75 eq). The reaction mixture was stirred at 15° C. for 15 hours and then concentrated in vacuo. The residue was re-dissolved in dichloromethane (200 mL). The resulting solution was added into MeOH (1.2 L) and a solid precipitated. The suspension was filtered and the filtrate was concentrated in vacua. The residue was re-dissolved in dichloromethane (150 mL). Then the resulting solution was added into tert-butyl methyl ether (700 mL) and a solid precipitated. The suspension was filtered and the filter cake was dried to give the title compound (32 g, 81%) as a white solid.

¹H NMR (DMSO-d₆): δ 7.50 (s, 2H), 6.81 (s, 1H), 3.89 (s, 3H) and 3.02 (d, 6H).

LCMS: m/z 233.2 (M+H)⁺ (ES⁺).

### Intermediate P37: ((1-Cyclopropyl-1H-pyrazol-3-yl)sulfonyl)(4-(dimethylamino) pyridin-1-ium-1-carbonyl)amide

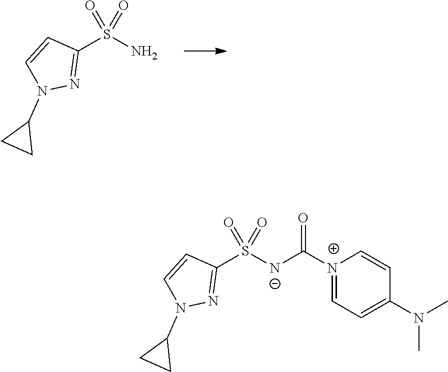 [SMILES: NS(=O)(=O)c1ccn(C2CC2)n1 ; CN(C)c1cc[n+](C(=O)[N-]S(=O)(=O)c2ccn(C3CC3)n2)cc1]

A mixture of 1-cyclopropyl-1H-pyrazole-3-sulfonamide (1.35 g, 7.21 mmol) and N,N-dimethylpyridin-4-amine (1.762 g, 14.42 mmol) in anhydrous MeCN (15 mL) was stirred at room temperature for 10 minutes. Then diphenyl carbonate (1.70 g, 7.93 mmol) was added and the reaction was stirred for 16 hours. The solid obtained was collected by filtration and rinsed with MTBE (5 mL) to afford the title compound as a solid (1.57 g, 55%).

¹H NMR (DMSO-d₆) δ 8.82-8.63 (m, 2H), 7.81 (d, J=2.3 Hz, 1H), 7.04-6.86 (m, 2H), 6.57 (d, J=2.4 Hz, 1H), 3.76 (m, 1H), 3.25 (s, 6H), 1.07-1.01 (m, 2H), 1.00-0.95 (m, 2H).

### Intermediate P38: 1-Cyclobutyl-1H-pyrazole-3-sulfonamide

### Step A: Lithium 1-(tetrahydro-2H-pyran-2-yl)-1H-pyrazole-5-sulfinate

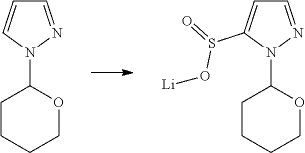

A solution of n-BuLi (100 mL, 250 mmol, 2.5M in hexanes) was added slowly to a solution of 1-(tetrahydro-2H-pyran-2-yl)-1H-pyrazole (36.2 g, 238 mmol) in THF (500 mL), keeping the temperature below −65° C. The mixture was stirred for 1.5 hours, then sulfur dioxide was bubbled through for 10 minutes. The mixture was allowed to warm to room temperature, the solvent evaporated and the residue triturated with TBME (300 mL) and filtered. The solid was washed with TBME and isohexane and dried to afford the crude title compound (54.89 g, 99%).

¹H NMR (DMSO-d6) δ 7.26 (d, J=1.6 Hz, 1H), 6.10 (d, J=1.7 Hz, 1H), 5.99 (dd, J=10.0, 2.5 Hz, 1H), 3.92-3.87 (m, 1H), 3.56-3.49 (m, 1H), 2.25-2.15 (m, 1H), 2.00-1.91 (m, 1H), 1.75-1.69 (m, 1H), 1.66-1.46 (m, 3H).

LCMS; m/z 215 (M−H)⁻ (ES).

### Step B: N,N-Bis(4-methoxybenzyl)-1-(tetrahydro-2H-pyran-2-yl)-1H-pyrazole-5-sulfonamide

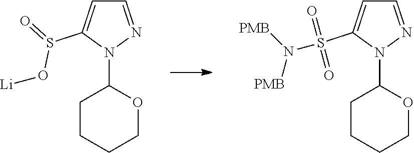

NCS (12.0 g, 90 mmol) was added to a suspension of lithium 1-(tetrahydro-2H-pyran-2-yl)-1H-pyrazole-5-sulfinate (20 g, 90 mmol) in DCM (250 mL) cooled in an ice bath. The mixture was stirred for 4 hours, quenched with water (100 mL), and then partitioned between DCM (300 mL) and water (200 mL). The organic phase was washed with water (200 mL), dried (MgSO₄), filtered and evaporated to ˜50 mL. The solution was added to a mixture of bis(4-methoxybenzyl)amine (24 g, 93 mmol) and triethylamine (40 mL, 287 mmol) in DCM (300 mL) cooled in an ice bath. After stirring for 1 hour, the mixture was warmed to room temperature, and then partitioned between DCM (300 mL) and water (250 mL). The organic layer was washed with water (250 mL), aq 1M HCl (2×250 mL), water (250 mL), dried (MgSO₄), filtered, and evaporated to afford the crude title compound (41.02 g, 97%) as a brown oil.

LCMS; m/z 494.2 (M+Na)⁺ (ES⁺).

### Step C: N,N-Bis(4-methoxybenzyl)-1H-pyrazole-3-sulfonamide

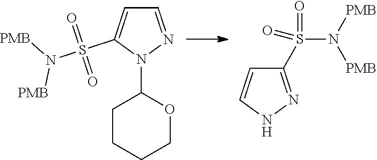

A mixture of N,N-bis(4-methoxybenzyl)-1-(tetrahydro-2H-pyran-2-yl)-1H-pyrazole-5-sulfonamide (41 g, 87 mmol) and aq 1M HCl (30 mL) in THF (300 mL) and MeOH (50 mL) was stirred at room temperature for 18 hours. The solvent was evaporated and the residue partitioned between EtOAc (400 mL) and aq 1M HCl (200 mL). The organic layer was washed with 10% brine (200 mL), dried (MgSO₄), filtered and evaporated. The residue was triturated with TBME, filtered and dried to afford the title compound (24.87 g, 69%) as an off white solid.

¹H NMR (CDCl₃) δ 7.88 (d, J=2.4 Hz, 1H), 7.06-7.02 (m, 4H), 6.79-6.75 (m, 4H), 6.63 (d, J=2.4 Hz, 1H), 4.31 (s, 4H), 3.78 (s, 6H). Exchangeable proton not visible.

LCMS; m/z 388 (M+H)⁺ (ES⁺); 386 (M−H)⁻ (ES⁻).

### Step D: 1-Cyclobutyl-N,N-bis(4-methoxybenzyl)-1H-pyrazole-3-sulfonamide

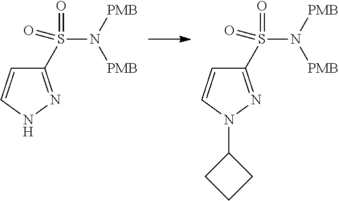

A solution of N,N-bis(4-methoxybenzyl)-1H-pyrazole-3-sulfonamide (5 g, 12.90 mmol) in DMF (60 mL) was cooled to 0° C., before sodium hydride (0.671 g, 16.78 mmol) was added. The mixture was warmed to room temperature and stirred for 30 minutes, before bromocyclobutane (1.3 ml, 13.81 mmol) was added slowly via syringe. The resulting mixture was stirred at 50° C. over the weekend. The mixture was diluted with EtOAc (100 mL). H₂O (100 mL) was added and the layers were separated. The aqueous layer was extracted with EtOAc (2×100 mL) and the combined organic extracts were washed with brine (3×80 mL), passed through a phase separator and concentrated in vacua. The residue was loaded onto silica and purified by chromatography (80 g column, 0-100% EtOAc/isohexane) to afford the title compound (4.72 g, 75%) as a pale yellow oil.

¹H NMR (DMSO-d6) δ 8.03 (d, J=2.4 Hz, 1H), 7.04 (d, J=8.6 Hz, 4H), 6.81 (d, J=8.6 Hz, 4H), 6.71 (d, J=2.3 Hz, 1H), 4.94 (p, J=8.4 Hz, 1H), 4.22 (s, 4H), 3.72 (s, 6H), 2.49-2.38 (m, 4H), 1.87-1.77 (m, 2H).

LCMS; m/z 464.2 (M+Na)⁺ (ES⁺).

### Step E: 1-Cyclobutyl-1H-pyrazole-3-sulfonamide

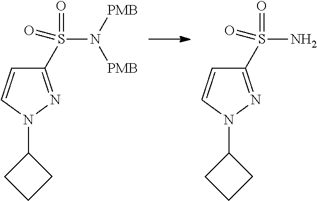

1-Cyclobutyl-N,N-bis(4-methoxybenzyl)-1H-pyrazole-3-sulfonamide (4.72 g, 10.69 mmol) was dissolved in TFA (5 mL) and DCM (5 mL) and stirred overnight at room temperature. The reaction mixture was concentrated in vacua and the residue was purified by chromatography on silica gel (40 g cartridge, 0-10% MeOH/DCM) to afford the title compound (1.5 g, 66%) as a pale white solid.

¹H NMR (DMSO-d6) δ 7.96 (d, J=2.4 Hz, 1H), 7.39 (s, 2H), 6.59 (d, J=2.4 Hz, 1H), 4.96-4.86 (m, 1H), 2.50-2.44 (m, 2H), 2.44-2.36 (m, 2H), 1.85-1.77 (m, 2H).

LCMS; m/z 202.0 (M+H)⁺ (ES⁺).

### Intermediate P39: 1-(1-(Azetidin-1-yl)-2-methylpropan-2-yl)-1H-pyrazole-3-sulfonamide

### Step A: Methyl 2-(3-(N,N-bis(4-methoxybenzyl)sulfamoyl)-1H-pyrazol-1-yl)-2-methylpropanoate

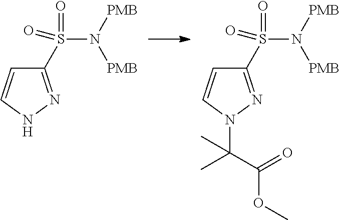

N,N-Bis(4-methoxybenzyl)-1H-pyrazole-3-sulfonamide (2.00 g, 5.16 mmol) (Intermediate P38, Step C) and potassium carbonate (2.140 g, 15.49 mmol) were suspended in dry DMF (30 mL). Methyl 2-bromo-2-methylpropanoate (1.002 mL, 7.74 mmol) was added and the mixture was heated to 80° C. overnight. The reaction mixture was cooled to room temperature, diluted with water (20 mL), poured into brine (200 mL) and extracted with MTBE (2×50 mL). The combined organic layers were dried (MgSO₄), filtered and evaporated to dryness to give a yellow oil. The crude product was purified by chromatography on silica gel (80 g column, 0-70% EtOAc/isohexane) to afford the title compound (2.45 g, 94%) as a clear colourless oil.

¹H NMR (DMSO-d₆) δ 8.18 (d, J=2.5 Hz, 1H), 7.05-6.95 (m, 4H), 6.85-6.78 (m, 4H), 6.78 (d, J=2.5 Hz, 1H), 4.18 (s, 4H), 3.72 (s, 6H), 3.65 (s, 3H), 1.81 (s, 6H).

LCMS; m/z 511 (M+Na)+ (ES+).

### Step B: 2-(3-(N,N-Bis(4-methoxybenzyl)sulfamoyl)-1H-pyrazol-1-yl)-2-methylpropanoic acid

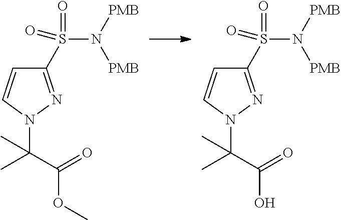

A mixture of methyl 2-(3-(N,N-bis(4-methoxybenzyl)sulfamoyl)-1H-pyrazol-1-yl)-2-methylpropanoate (2.4 g, 4.92 mmol) and aq 2 M NaOH (5 mL, 10.00 mmol) in THF (5 mL) and MeOH (3 mL) was stirred at room temperature for 20 hours. The mixture was partitioned between EtOAc (100 mL) and aq 1 M HCl (100 mL). The organic layer was washed with brine (50 mL), dried (MgSO₄), filtered and evaporated to afford the title compound (2.38 g, 95%) as a gum that solidified on standing.

¹H NMR (CDCl₃) δ 7.64 (d, J=2.5 Hz, 1H), 7.09-7.05 (m, 4H), 6.80-6.77 (m, 4H), 6.73 (d, J=2.5 Hz, 1H), 4.32 (s, 4H), 3.80 (s, 6H), 1.91 (s, 6H). Exchangeable proton not visible.

LCMS; m/z 472 (M−H)− (ES−).

### Step C: 1-(1-(Azetidin-1-yl)-2-methyl-1-oxopropan-2-yl)-N,N-bis(4-methoxy benzyl)-1H-pyrazole-3-sulfonamide

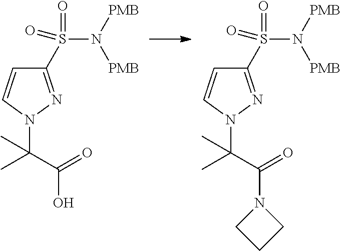

A mixture of 2-(3-(N,N-bis(4-methoxybenzyl)sulfamoyl)-1H-pyrazol-1-yl)-2-methylpropanoic acid (1.15 g, 2.234 mmol), Hunig's base (1.557 ml, 8.91 mmol) and HATU (0.921 g, 2.422 mmol) in DMF (6.5 ml) was stirred at 0-5° C. for 10 minutes. Then azetidine HCl (0.272 g, 2.90 mmol) was added. The mixture was allowed to warm to room temperature and stirred for 20 hours. Additional HATU (0.263 g, 1.117 mmol) was added, followed by Hunig's base (0.390 ml, 2.234 mmol). The mixture was cooled to 0-5° C. for 10 minutes. Then additional azetidine HCl (0.064 g, 1.117 mmol) was added. The mixture was allowed to warm to room temperature, stirred for a further hour, and then partitioned between TBME (75 ml) and water (40 ml). The organic layer was washed with aq 1M HCl (40 ml), water (25 ml), dried (MgSO₄), filtered, evaporated, and then purified by chromatography on silica gel (120 g column, 0-100% TBME/isohexane) to afford the title compound (615 mg, 51%) as a clear gum.

¹H NMR (CDCl₃) δ 7.56 (d, J=2.4 Hz, 1H), 7.13-7.09 (m, 4H), 6.80-6.76 (m, 5H), 4.32 (s, 4H), 3.99 (t, J=7.8 Hz, 2H), 3.79 (s, 6H), 3.23 (t, J=7.7 Hz, 2H), 2.08-2.01 (m, 2H), 1.78 (s, 6H).

LCMS; m/z 513.1 (M+H)⁺ (ES⁺).

### Step D: 1-(1-(Azetidin-1-yl)-2-methylpropan-2-yl)-N,N-bis(4-methoxybenzyl)-1H-pyrazole-3-sulfonamide

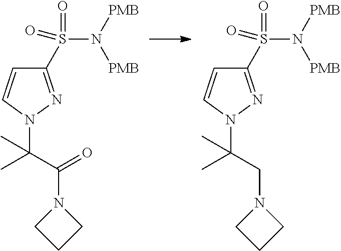

BH₃.THF (1 M in THF) (21.53 ml, 21.53 mmol) was added to a solution of 1-(1-(azetidin-1-yl)-2-methyl-1-oxopropan-2-yl)-N,N-bis(4-methoxybenzyl)-1H-pyrazole-3-sulfonamide (3.1537 g, 6.15 mmol) in THF (26.3 mL). The mixture was stirred for 3 minutes, and then heated to reflux over the weekend. The reaction was allowed to cool to room temperature, before being placed in an ice-bath. MeOH (50 mL) was added dropwise and the mixture was heated at 60° C. for 3 hours, and then allowed to cool to room temperature overnight. The mixture was concentrated under reduced pressure and loaded onto a column of SCX (30 g) in MeOH (50 mL). The column was washed with MeOH (100 mL), 0.7 M ammonia in MeOH (100 mL), and then the product was eluted with 7 M ammonia in MeOH (100 mL). The resultant mixture was concentrated in vacuo to afford the title compound (2.89 g, 85%) as a colourless viscous oil.

¹H NMR (DMSO-d6) δ=7.98 (d, J=2.5 Hz, 1H), 7.07-7.02 (m, 4H), 6.84-6.79 (m, 4H), 6.69 (d, J=2.4 Hz, 1H), 4.19 (s, 4H), 3.72 (s, 6H), 2.92 (t, J=7.0 Hz, 4H), 2.68 (s, 2H), 1.84 (p, J=7.0 Hz, 2H), 1.48 (s, 6H).

LCMS; m/z 499.2 (M+H)⁺ (ES⁺).

### Step E: 1-(1-(Azetidin-1-yl)-2-methylpropan-2-yl)-1H-pyrazole-3-sulfonamide

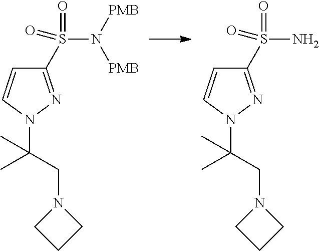

1-(1-(Azetidin-1-yl)-2-methylpropan-2-yl)-N,N-bis(4-methoxybenzyl)-1H-pyrazole-3-sulfonamide (2.89 g, 5.80 mmol) was dissolved in TFA (15 mL) and DCM (15 mL) and allowed to stir overnight. Additional TFA (5 ml, 5.80 mmol) was added and the reaction stirred at room temperature for 3 hours. The reaction mixture was concentrated in vacua, MeOH (50 mL) was added, the precipitate was filtered off and the filtrate loaded onto a column of SCX (30 g). The column was washed with MeOH (100 mL). The product was then eluted with 7N NH₃ in MeOH (100 mL) and concentrated in vacua. The product was purified by chromatography on silica gel (40 g column, 0-10% MeOH/DCM) to afford the title compound (1.06 g, 69%) as a white solid.

¹H NMR (DMSO-d6) δ 7.89 (d, J=2.5 Hz, 1H), 7.34 (s, 2H), 6.54 (d, J=2.4 Hz, 1H), 2.94 (t, J=7.0 Hz, 4H), 2.68 (s, 2H), 1.84 (p, J=7.0 Hz, 2H), 1.47 (s, 6H).

LCMS; m/z 259.1 (M+H)⁺ (ES⁺).

### Intermediate A1: 4-Fluoro-2-isopropyl-6-(pyridin-3-yl)aniline

### Step A: 2-Bromo-4-fluoro-6-iso-propylaniline

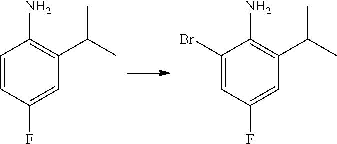

N-Bromosuccinimide (5.64 g, 31.7 mmol) was added portionwise to 4-fluoro-2-isopropylaniline (4.62 g, 30.2 mmol) in dichloromethane (72 mL) at 0° C. The resulting mixture was stirred at 0° C. for 1 hour and then left to warm to room temperature over 21 hours. The reaction mixture was washed with a solution of aqueous sodium hydroxide (2 M, 2×50 mL), dried (magnesium sulfate), filtered and concentrated in vacua to give a brown residue. The crude product was then filtered through a plug of silica (50 g) and washed through with 50% dichloromethane in iso-hexane (500 mL). The red filtrate was concentrated to dryness and the crude product was purified by chromatography on silica gel (120 g column, 0-10% dichloromethane/iso-hexane) to afford the title compound (4.99 g, 70%) as a red oil.

¹H NMR (CDCl₃) δ 7.07 (dd, 1H), 6.86 (dd, 1H), 4.14 (s, 2H), 2.93 (sep, 1H) and 1.25 (d, 6H).

LCMS m/z 232.2/234.3 (M+H)⁺ (ES⁺).

### Step B: 4-Fluoro-2-isopropyl-6-(pyridin-3-yl)aniline

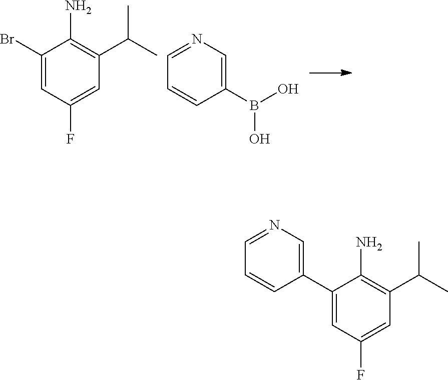

To a stirred, nitrogen-degassed mixture of 2-bromo-4-fluoro-6-iso-propylaniline (1.00 g, 4.27 mmol) was added pyridin-3-ylboronic acid (0.577 g, 4.69 mmol), [1,1′bis(diphenylphosphino)ferrocene] dichloropalladium(II) (Pd(dppf)Cl₂, 0.156 g, 0.213 mmol) and potassium carbonate (1.769 g, 12.80 mmol) in a 10:1 mixture of 1,4-dioxane:water (33 mL). The reaction mixture was then heated to 80° ° C. under a nitrogen atmosphere for 2 days, left to cool to room temperature, filtered through a pad of Celite (10 g) and the filter cake washed with ethyl acetate (2×30 mL). The filtrate was poured onto water (50 mL) and the organic layer collected. The aqueous layer was extracted with ethyl acetate (2×20 mL) and the combined organic layers were dried (magnesium sulfate), filtered and evaporated to dryness. The crude product was purified by chromatography on silica gel (80 g column, 0-60% ethyl acetate/iso-hexane) to afford the title compound (273 mg, 27%) as a brown gum.

¹H NMR (CDCl₃) δ 8.70 (dd, 1H), 8.63 (dd, 1H), 7.82 (ddd, 1H), 7.48-7.34 (m, 1H), 6.94 (dd, 1H), 6.70 (dd, 1H), 2.93 (sept, 1H), 3.98-2.44 (br s, 2H) and 1.29 (d, 6H).

LCMS m/z 231.1 (M+H)⁺ (ES⁺).

The following intermediates were synthesised following the general procedure for Intermediate A1:

| Intermediate | Structure | Analytical data |
|---|---|---|
| A2 | 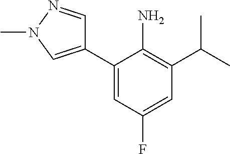 [SMILES: CC(C)c1cc(F)cc(-c2cnn(C)c2)c1N] | ¹H NMR (CDCl₃) δ 7.68 (d, 1 H), 7.58 (d, 1 H), 6.86 (dd, 1 H), 6.78 (dd, 1 H), 3.99 (s, 3 H), 3.74 (br s, 2 H), 2.94 (sept, 1 H) and 1.29 (d, 6 H). (85 mg, 22%) |
| | 4-Fluoro-2-isopropyl-6-(1- | |
| | methyl-1H-pyrazol-4- | |
| | yl)aniline | |
| A3 | 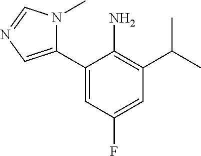 [SMILES: CC(C)c1cc(F)cc(-c2cncn2C)c1N] | ¹H NMR (CDCl₃) δ 7.68 (s, 1 H), 7.20 (s, 1 H), 6.94 (dd, 1 H), 6.67 (dd, 1 H), 3.53 (s, 3 H), 2.94-2.82 (m, 1 H), 2.47 (s, 2 H) and 1.27 (d, 6H). LCMS m/z 234.1 (M + H)⁺ (ES⁺). (56 mg, 13%) |
| | 4-Fluoro-2-isopropyl-6-(1- | |
| | methyl-1H-imidazol-5- | |
| | yl)aniline | |
| A4 | 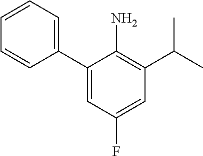 [SMILES: CC(C)c1cc(F)cc(-c2ccccc2)c1N] | ¹H NMR (CDCl₃) δ 7.50-7.32 (m, 5 H), 6.90 (dd, 1 H), 6.74 (dd, 1 H), 4.11 (br s, 2 H), 3.15- 2.80 (m, 1 H) and 1.29 (d, 6 H). LCMS m/z 230.1 (M + H)⁺ (ES⁺). (161 mg, 82%) |
| | 5-Fluoro-3-isopropyl-[1,1′- | |
| | biphenyl]-2-amine | |
| A5 | 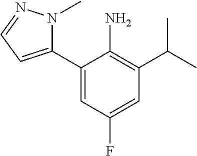 [SMILES: CC(C)c1cc(F)cc(-c2ccnn2C)c1N] | ¹H NMR (CDCl₃) δ 7.69 (d, 1 H), 7.01 (dd, 1 H), 6.71 (dd, 1 H), 6.42 (d, 1 H), 3.85 (s, 3 H), 2.94 (sept, 1 H) and 1.29 (d, 6 H). LCMS m/z 234.1 (M + H)⁺ (ES⁺). (125 mg, 57%) |
| | 4-Fluoro-2-isopropyl-6-(1- | |
| | methyl-1H-pyrazol-5- | |
| | yl)aniline | |
| A6 | 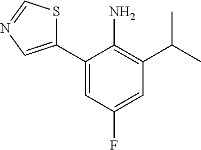 [SMILES: CC(C)c1cc(F)cc(-c2cncs2)c1N] | ¹H NMR (CDCl₃) δ 8.87 (s, 1 H), 7.97 (s, 1 H), 6.93 (dd, 1 H), 6.83 (dd, 1 H), 3.80 (s, 2 H), 2.92 (sept, 1 H) and 1.28 (d, 6 H). LCMS m/z 237.1 (M + H)⁺ (ES⁺). (23 mg, 7%) |
| | 4-Fluoro-2-isopropyl-6- | |
| | (thiazol-5-yl)aniline | |
| A7 | 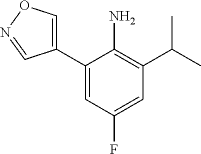 [SMILES: CC(C)c1cc(F)cc(-c2cnoc2)c1N] | ¹H NMR (CDCl₃) δ 8.69 (s, 1 H), 8.52 (s, 1 H), 6.95 (dd, 1 H), 6.78 (dd, 1 H), 3.09-2.90 (m, 1 H), 1.48 (s, 2 H) and 1.29 (d, 6 H). LCMS m/z 221.1 (M + H)⁺ (ES⁺). (40 mg, 20%) |
| | 4-Fluoro-2-isopropyl-6- | |
| | (isoxazol-4-yl)aniline | |
| A8 | 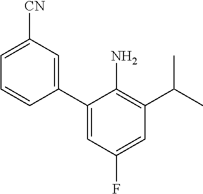 [SMILES: CC(C)c1cc(F)cc(-c2cccc(C#N)c2)c1N] | ¹H NMR (CDCl₃) δ 7.82-7.74 (m, 1 H), 7.73- 7.66 (m, 1 H), 7.66-7.60 (m, 1 H), 7.59-7.49 (m, 1 H), 6.96 (dd 1 H), 6.69 (dd, 1 H), 3.10- 2.84 (m, 1 H) and 1.29 (d, 6 H). LCMS m/z 255.1 (M + H)⁺ (ES⁺). (182 mg, 81%) |
| | 2′-Amino-5′-fluoro-3′- | |
| | isopropyl-[1,1′-biphenyl]-3- | |
| | carbonitrile | |
| A9 | 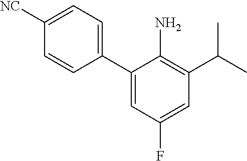 [SMILES: CC(C)c1cc(F)cc(-c2ccc(C#N)cc2)c1N] | ¹H NMR (CDCl₃) δ 7.97-7.86 (m, 2 H), 7.74- 7.52 (m, 2 H), 6.94 (dd, 1 H), 6.73 (dd, 1 H), 4.46 (s, 2 H), 3.19-2.97 (m, 1 H) and 1.19 (d, 6H). LCMS m/z 255.1 (M + H)⁺ (ES⁺). (189 mg, 83%) |
| | 2′-Amino-5′-fluoro-3′- | |
| | isopropyl-[1,1′-biphenyl]-4- | |
| | carbonitrile | |
| A10 | 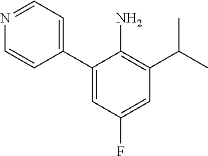 [SMILES: CC(C)c1cc(F)cc(-c2ccncc2)c1N] | ¹H NMR (CDCl₃) δ 8.72-8.65 (m, 2 H), 7.50- 7.42 (m, 2 H), 6.95 (dd, 1 H), 6.72 (dd, 1 H), 3.39 (br s, 2 H), 3.00-2.85 (m, 1 H) and 1.29 (d, 6 H). LCMS m/z 231.1 (M + H)⁺ (ES⁺). (148 mg, 75%) |
| | 4-Fluoro-2-isopropyl-6- | |
| | (pyridin-4-yl)aniline | |
| A11 | 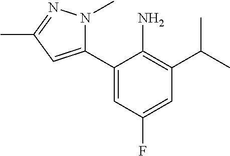 [SMILES: Cc1cc(-c2cc(F)cc(C(C)C)c2N)n(C)n1] | ¹H NMR (CDCl₃) δ 6.95 (dd, 1 H), 6.68 (dd, 1 H), 6.09 (s, 1 H), 3.69 (s, 3 H), 2.98-2.81 (m, 1 H), 2.33 (s, 3 H) and 1.28 (d, 6 H). LCMS m/z 248.1 (M + H)⁺ (ES⁺). (72 mg, 34%) |
| | 2-(1,3-Dimethyl-1H- | |
| | pyrazol-5-yl)-4-fluoro-6- | |
| | isopropylaniline | |
| A12 | 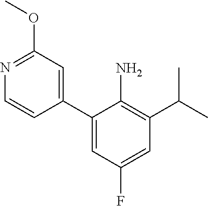 [SMILES: COc1cc(-c2cc(F)cc(C(C)C)c2N)ccn1] | ¹H NMR (CDCl₃) δ 8.25 (d, 1 H), 7.00 (dd, 1 H), 6.93 (dd, 1 H), 6.85 (s, 1 H), 6.71 (dd, 1 H), 4.01 (s, 3 H), 2.92 (sept, 1 H) and 1.28 (d, 6 H). Exchangeable NH₂ observed as broad signal from 4.5-0.5 ppm. LCMS m/z 261.1 (M + H)⁺ (ES⁺). (174 mg, 78%) |
| | 4-Fluoro-2-isopropyl-6-(2- | |
| | methoxypyridin-4- | |
| | yl)aniline | |
| A13 | 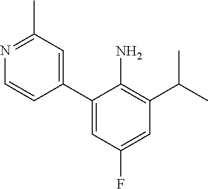 [SMILES: Cc1cc(-c2cc(F)cc(C(C)C)c2N)ccn1] | ¹H NMR (CDCl₃) δ 8.57 (dd, 1 H), 7.29 (d, 1 H), 7.25-7.22 (m, 1H), 6.93 (dd, 1 H), 6.70 (dd, 1 H), 3.62 (br s, 2 H), 2.92 (sept, 1 H), 2.64 (s, 3 H) and 1.29 (d, 6 H). LCMS m/z 245.1 (M + H)⁺ (ES⁺). (130 mg, 62%) |
| | 4-Fluoro-2-isopropyl-6-(2- | |
| | methylpyridin-4-yl)aniline | |
| A14 | 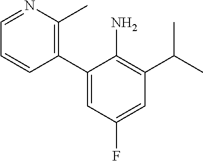 [SMILES: Cc1ncccc1-c1cc(F)cc(C(C)C)c1N] | ¹H NMR (CDCl₃) δ 8.57 (s, 1 H), 7.64 (d, 1 H), 7.31 (s, 1 H), 6.94 (dd, 1 H), 6.60 (dd, 1 H), 3.33 (s, 2 H), 2.92 (sept, 1 H), 2.48 (s, 3 H) and 1.29 (dd, 6 H). LCMS m/z 245.1 (M + H)⁺ (ES⁺). (104 mg, 44%) |
| | 4-Fluoro-2-isopropyl-6-(2- | |
| | methylpyridin-3-yl)aniline | |
| A15 | 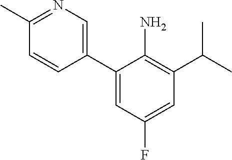 [SMILES: Cc1ccc(-c2cc(F)cc(C(C)C)c2N)cn1] | ¹H NMR (CDCl₃) δ 8.58 (d, 1 H), 7.73 (dd, 1 H), 7.29 (d, 1 H), 6.92 (dd, 1 H), 6.69 (dd, 1 H), 3.54 (br s, 2 H), 3.00-2.85 (m, 1 H), 2.65 (s, 3 H) and 1.29 (d, 6 H). LCMS m/z 245.1 (M + H)⁺ (ES⁺). (211 mg, 95%) |
| | 4-Fluoro-2-isopropyl-6-(6- | |
| | methylpyridin-3-yl)aniline | |
| A16 | 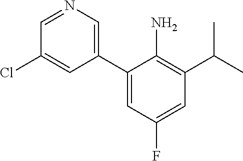 [SMILES: CC(C)c1cc(F)cc(-c2cncc(Cl)c2)c1N] | ¹H NMR (CDCl₃) δ 8.62-8.56 (m, 2 H), 7.83 (t, 1 H), 6.96 (dd, 1 H), 6.69 (dd, 1 H), 3.46- 3.02 (br s, 2 H), 2.93 (sept, 1 H) and 1.29 (d, 6 H). LCMS m/z 265.1/267.1 (M + H)⁺ (ES⁺). (150 mg, 53%) |
| | 2-(5-Chloropyridin-3-yl)-4- | |
| | fluoro-6-isopropylaniline | |
| A17 | 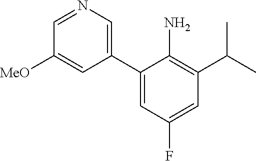 [SMILES: COc1cncc(-c2cc(F)cc(C(C)C)c2N)c1] | ¹H NMR (CDCl₃) δ 8.34 (d, 1 H), 8.33 (d, 1 H), 7.45 (dd, 1 H), 6.96 (dd, 1 H), 6.71 (dd, 1 H), 3.93 (s, 3 H), 2.92 (sept, 1 H), 1.29 (d, 6 H). Exchangeable NH₂ signal not observed LCMS m/z 261.2 (M + H)⁺ (ES⁺). (146 mg, 61%) |
| | 4-Fluoro-2-isopropyl-6-(5- | |
| | methoxypyridin-3-yl)aniline | |
| A18 | 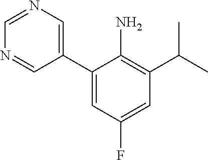 [SMILES: CC(C)c1cc(F)cc(-c2cncnc2)c1N] | ¹H NMR (CDCl₃) δ 9.23 (s, 1 H), 8.86 (s, 2 H), 6.98 (dd, 1 H), 6.69 (dd, 1 H), 3.55 (br s, 2 H), 2.92 (sept, 1 H) and 1.29 (d, 6 H). (126 mg, 60%) |
| | 4-Fluoro-2-isopropyl-6- | |
| | (pyrimidin-5-yl)aniline | |
| A19 | 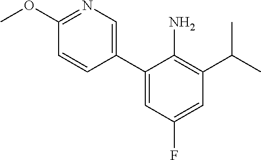 [SMILES: COc1ccc(-c2cc(F)cc(C(C)C)c2N)cn1] | ¹H NMR (CDCl₃) δ 8.25 (d, 1 H), 7.71 (dd, 1 H), 6.93 (dd, 1 H), 6.87 (d, 1 H), 6.69 (dd, 1 H), 4.01 (s, 3 H), 3.08-2.90 (m, 1 H) and 1.29 (d, 6 H). Exchangeable NH₂ signal not observed LCMS m/z 261.4 (M + H)⁺ (ES⁺). (62 mg, 26%) |
| | 4-Fluoro-2-isopropyl-6-(6- | |
| | methoxypyridin-3-yl)aniline | |
| A20 | 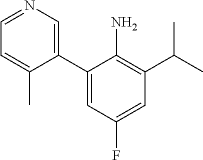 [SMILES: Cc1ccncc1-c1cc(F)cc(C(C)C)c1N] | ¹H NMR (CDCl₃) δ 8.54 (s, 1 H), 8.48 (s, 1 H), 7.75 (s, 1 H), 6.95 (dd, 1 H), 6.69 (dd, 1 H), 2.92 (sept, 1 H), 2.45 (s, 3 H) and 1.29 (d, 6 H). Exchangeable NH₂ signal not observed. LCMS m/z 245.4 (M + H)⁺ (ES⁺). (70 mg, 29%) |
| | 4-Fluoro-2-isopropyl-6-(4- | |
| | methylpyridin-3-yl)aniline | |
| A21 | 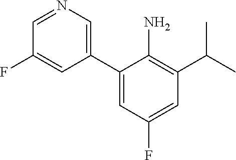 [SMILES: CC(C)c1cc(F)cc(-c2cncc(F)c2)c1N] | ¹H NMR (CDCl₃) δ 8.56 (t, 1 H), 8.50 (d, 1 H), 7.60 (ddd, 1 H), 6.97 (dd, 1 H), 6.71 (dd, 1 H), 2.95 (sept, 1 H), 3.26-2.49 (br s, 2 H) and 1.29 (d, 6 H). LCMS m/z 249.2 (M + H)⁺ (ES⁺). (58 mg, 26%) |
| | 4-Fluoro-2-(5- | |
| | fluoropyridin-3-yl)-6- | |
| | isopropylaniline | |
| A22 | 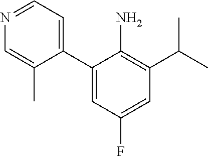 [SMILES: Cc1cnccc1-c1cc(F)cc(C(C)C)c1N] | ¹H NMR (CDCl₃) δ 8.56 (s, 1 H), 8.51 (d, 1 H), 7.33 (d, 1 H), 6.94 (dd, 1 H), 6.55 (dd, 1 H), 2.88 (sept, 1 H), 2.22 (s, 3 H) and 1.26 (d, 6 H). Exchangeable NH₂ signal not observed. LCMS m/z 245 (M + H)⁺ (ES⁺). (225 mg, 54%) |
| | 4-Fluoro-2-isopropyl-6-(3- | |
| | methylpyridin-4-yl)aniline | |
| A23 | 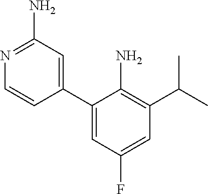 [SMILES: CC(C)c1cc(F)cc(-c2ccnc(N)c2)c1N] | ¹H NMR (CDCl₃) δ 8.10-7.90 (m, 1 H), 6.94 (dd, 1 H), 6.85-6.78 (m, 1 H), 6.76-6.66 (m, 2 H), 5.55 (br s, 2 H), 3.68 (br s, 2 H), 2.91 (sept, 1 H) and 1.28 (d, 6 H). LCMS m/z 246.4 (M + H)⁺ (ES⁺). (70 mg, 26%) |
| | 4-(2-Amino-5-fluoro-3- | |
| | isopropylphenyl)pyridin-2- | |
| | amine | |
| A24 | 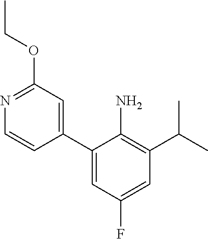 [SMILES: CCOc1cc(-c2cc(F)cc(C(C)C)c2N)ccn1] | ¹H NMR (CDCl₃) δ 8.22 (d, 1 H), 6.97 (dd, 1 H), 6.92 (dd, 1 H), 6.83 (s, 1 H), 6.71 (dd, 1 H), 4.42 (q, 2 H), 2.92 (sept, 1 H), 1.43 (t, 3 H) and 1.28 (d, 6 H). LCMS m/z 275.4 (M + H)⁺ (ES⁺). (203 mg, 80%) |
| | 2-(2-Ethoxypyridin-4-yl)-4- | |
| | fluoro-6-isopropylaniline | |
| A25 | 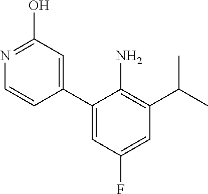 [SMILES: CC(C)c1cc(F)cc(-c2ccnc(O)c2)c1N] | ¹H NMR (DMSO-d₆) δ 11.55 (s, 1 H), 7.45- 7.38 (m, 1 H), 6.92 (dd, 1 H), 6.71 (dd, 1 H), 6.30-6.27 (m, 1 H), 6.20 (dd, 1 H), 4.50 (s, 2 H), 3.06 (sept, 1 H) and 1.17 (d, 6 H). LCMS m/z 247 (M + H)⁺ (ES⁺); 245 (M − H)⁻ (ES⁻). (40 mg, 11%) |
| | 4-(2-Amino-5-fluoro-3- | |
| | isopropylphenyl)pyridin-2- | |
| | ol | |
| A26 | 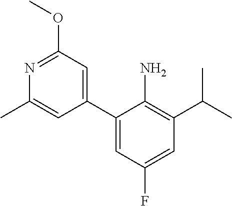 [SMILES: COc1cc(-c2cc(F)cc(C(C)C)c2N)cc(C)n1] | ¹H NMR (CDCl₃) δ 6.91 (dd, 1 H), 6.81 (dd, 1 H), 6.69 (dd, 1 H), 6.62 (dd, 1 H), 4.08-3.39 (br s, 2 H), 3.96 (s, 3 H), 2.91 (sept, 1 H), 2.50 (d, 3 H) and 1.28 (d, 6 H). LCMS m/z 275.4 (M + H)⁺ (ES⁺). (228 mg, 85%) |
| | 4-Fluoro-2-isopropyl-6-(2- | |
| | methoxy-6-methylpyridin- | |
| | 4-yl)aniline | |
| A27 | 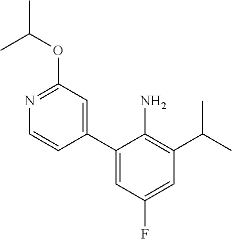 [SMILES: CC(C)Oc1cc(-c2cc(F)cc(C(C)C)c2N)ccn1] | ¹H NMR (CDCl₃) δ 8.21 (dd, 1 H), 6.99-6.83 (m, 2 H), 6.76 (dd, 1 H), 6.71 (dd, 1 H), 5.34 (sept, 1 H), 3.75 (s, 2 H), 2.92 (sept, 1 H), 1.38 (d, 6 H) and 1.28 (d, 6 H). LCMS m/z 289.4 (M + H)⁺ (ES⁺). (214 mg, 85%) |
| | 4-Fluoro-2-(2-isopropoxy- | |
| | pyridin-4-yl)-6-isopropyl- | |
| | aniline | |
| A28 | 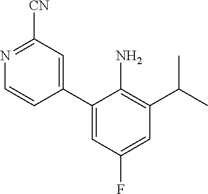 [SMILES: CC(C)c1cc(F)cc(-c2ccnc(C#N)c2)c1N] | ¹H NMR (CDCl₃) δ 8.78 (dd, 1 H), 7.86 (dd, 1 H), 7.65 (dd, 1 H), 6.99 (dd, 1 H), 6.69 (dd, 1 H), 3.49 (br s, 2 H), 2.93 (sept, 1 H) and 1.29 (d, 6 H). LCMS m/z 256.5 (M + H)⁺ (ES⁺). (89 mg, 29%) |
| | 4-(2-Amino-5-fluoro-3- | |
| | isopropylphenyl)picolino- | |
| | nitrile | |
| A29 | 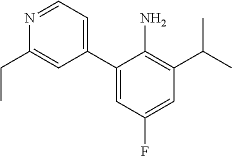 [SMILES: CCc1cc(-c2cc(F)cc(C(C)C)c2N)ccn1] | ¹H NMR (CDCl₃) δ 8.64 (dd, 1 H), 7.46-7.41 (m, 2 H), 6.99 (dd, 1 H), 6.75 (dd, 1 H), 3.68 (br s, 2 H), 3.04 (q, 2 H), 2.95 (sept, 1 H), 1.42 (t, 3 H) and 1.32 (d, 6 H). LCMS m/z 259 (M + H)⁺ (ES⁺); 257 (M − H)⁻ (ES⁻). (234 mg, 70%) |
| | 2-(2-Ethylpyridin-4-yl)-4- | |
| | fluoro-6-isopropylaniline | |
| A31 | 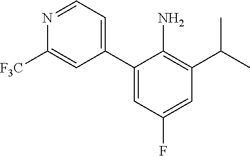 [SMILES: CC(C)c1cc(F)cc(-c2ccnc(C(F)(F)F)c2)c1N] | ¹H NMR (CDCl₃) δ 8.75 (s, 1H), 7.81 (d, J = 1.4 Hz, 1H), 7.64 (dd, J = 5.0, 1.5 Hz, 1H), 7.06 (dd, J = 9.9, 2.9 Hz, 1H), 6.77 (dd, J = 8.2, 2.9 Hz, 1H), 3.20-1.20 (br s, 2H), 3.05 (s, 1H), 1.32 (d, J = 6.7 Hz, 6H). LCMS m/z 299 (M + H)⁺ (ES⁺). (308 mg, 75%) |
| | 4-Fluoro-2-isopropyl-6-(2- | |
| | (trifluoromethyl)pyridin-4- | |
| | yl)aniline | |

### Intermediate A30: 4-Fluoro-2-isopropyl-6-(tetrahydro-2H-pyran-4-yl)aniline

### Step A: 2-Bromo-4-fluoro-6-(prop-1-en-2-yl)aniline

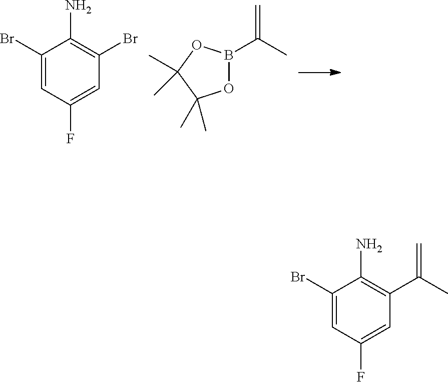

Nitrogen gas was bubbled through a mixture of 2,6-dibromo-4-fluoroaniline (5 g, 18.59 mmol), 4,4,5,5-tetramethyl-2-(prop-1-en-2-yl)-1,3,2-dioxaborolane (4.2 ml, 22.34 mmol) and potassium triphosphate (7.9 g, 37.2 mmol) in dioxane (50 mL) and water (8 mL) for 15 minutes, then (2-dicyclohexylphosphino-2′,4′,6′-triisopropyl-1,1′-biphenyl)[2-(2′-amino-1,1′-biphenyl)]palladium(II) methanesulfonate [XPhos G3 Pd cat (500 mg, 0.591 mmol)] was added. The mixture was heated at 90° C. for 8 hours and then partitioned between hexane (200 mL) and water (100 mL). The organic layer was dried (magnesium sulfate), filtered, evaporated in vacua and the residue purified by chromatography on silica gel (120 g column, 0-2% ethyl acetate/iso-hexane) to afford the title compound (1.95 g, 43%) as an oil.

¹H NMR (CDCl₃) δ 7.13 (dd, 1H), 6.77 (dd, 1H), 5.37-5.35 (m, 1H), 5.12-5.10 (m, 1H), 3.52 (br s, 2H) and 2.08-2.06 (m, 3H).

LCMS m/z 230.2 (M+H)⁺ (ES⁺).

### Step B: 2-(3,6-Dihydro-2H-pyran-4-yl)-4-fluoro-6-(prop-1-en-2-yl)aniline

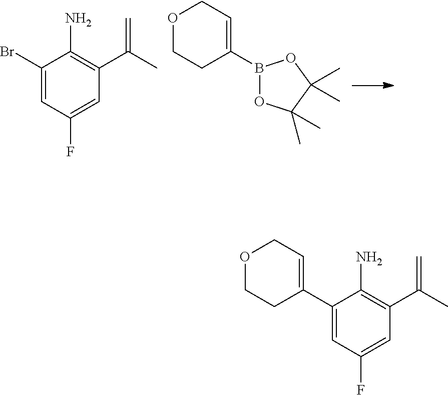

2-(3,6-Dihydro-2H-pyran-4-yl)-4,4,5,5-tetramethyl-1,3,2-dioxaborolane (457 mg, 2.176 mmol), tetrakis(triphenylphosphine)palladium(0) (251 mg, 0.218 mmol), sodium carbonate (923 mg, 8.70 mmol) and water (4 mL) were added to a sealed vialed containing a solution of 2-bromo-4-fluoro-6-(prop-1-en-2-yl)aniline (500 mg, 2.173 mmol) in N,N-dimethylformamide (22 mL). The reaction mixture was heated under nitrogen at 100° C. overnight and allowed to cool before the residue was diluted with ethyl acetate (50 mL), washed with brine (50 mL), dried (sodium sulfate) and concentrated in vacuo. The crude product was purified by chromatography on silica (40 g column, 0-20% ethyl acetate/iso-hexanes) to afford the title compound (355 mg, 65%) as a brownish oil.

¹H NMR (CDCl₃) δ 6.71 (dd, 1H), 6.67 (dd, 1H), 5.88 (m, 1H), 5.35-5.31 (m, 1H), 5.09 (m, 1H), 4.32 (m, 2H), 3.95 (t, 2H), 3.82 (br s, 2H), 2.42 (m, 2H) and 2.09-2.07 (m, 3H).

### Step C: 4-Fluoro-2-isopropyl-6-(tetrahydro-2H-pyran-4-yl)aniline

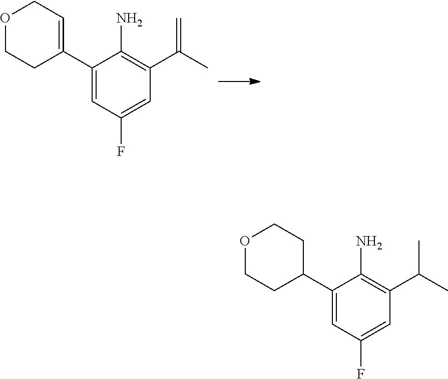 [SMILES: C=C(C)c1cc(F)cc(C2=CCOCC2)c1N ; CC(C)c1cc(F)cc(C2CCOCC2)c1N]

A mixture of 2-(3,6-dihydro-2H-pyran-4-yl)-4-fluoro-6-(prop-1-en-2-yl)aniline (355 mg, 1.522 mmol) and 5% palladium on carbon [156 mg, 0.03 mmol; type 87L (58.5% moisture)] in ethyl acetate (3.8 mL) was hydrogenated at 5 Bar for 1 hour. The mixture was filtered through Celite and evaporated to afford the title compound (340 mg, 91%).

¹H NMR (CDCl₃) δ 6.80 (dd, 1H), 6.75 (dd, 1H), 4.16-4.14 (m, 1H), 4.13-4.10 (m, 1H), 3.65-3.51 (m, 4l H), 3.01-2.89 (m, 1H), 2.85-2.74 (m, 1H), 1.86-1.78 (m, 4H) and 1.28 (d, 6H).

LCMS m/z 238.1 (M+H)⁺ (ES^{+).}

### Intermediate A32: 4-(2-Amino-5-fluoro-3-isopropylphenyl)-N,N-dimethylpyridin-2-amine

### Step A: 4-Fluoro-2-isopropyl-6-(4,4,5,5-tetramethyl-1,3,2-dioxaborolan-2-yl)aniline

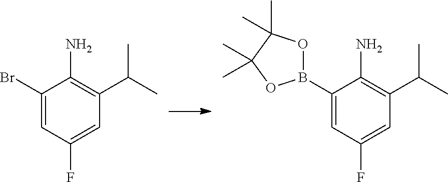 [SMILES: CC(C)c1cc(F)cc(Br)c1N ; CC(C)c1cc(F)cc(B2OC(C)(C)C(C)(C)O2)c1N]

In an oven dried round bottom flask, 2-bromo-4-fluoro-6-isopropylaniline (3.0 g, 12.93 mmol), 4,4,4′,4,5,5,5,5-octamethyl-2,2′-bi(1,3,2-dioxaborolane) (8.21 g, 32.3 mmol), KOAc (4.44 g, 45.2 mmol) and Pd(dppf)Cl₂. CH₂Cl₂ (2.11 g, 2.59 mmol) were added and the vessel was purged with nitrogen. Anhydrous 1,4-dioxane (86 mL) was added and the reaction was stirred at 110° C. for 2 hours. Upon completion, the reaction mixture was diluted with water, extracted with EtOAc (2×50 mL) and the combined organic extracts washed with brine (50 mL), dried and concentrated in vacuo. The crude product was purified by chromatography on silica (80 g column, 0-10% EtOAc/isohexane) then loaded onto a column of SCX (10 g) in acetonitrile. The column was washed with acetonitrile and then the product was eluted with 0.7 M ammonia in methanol. The resultant mixture was concentrated in vacua to afford the title compound (1.18 g, 32%) as a light yellow oil.

¹H NMR (CDCl₃) δ 7.21 (dd, J=8.7, 3.1 Hz, 1H), 6.96 (dd, J=10.0, 3.1 Hz, 1H), 4.72 (bs, 2H), 2.93-2.82 (m, 1H), 1.37 (s, 12H), 1.26 (d, J=6.8 Hz, 6H).

### Step B: 4-(2-Amino-5-fluoro-3-isopropylphenyl)-N,N-dimethylpyridin-2-amine

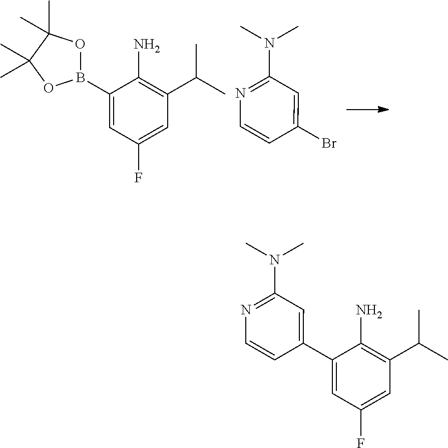

4-Fluoro-2-isopropyl-6-(4,4,5,5-tetramethyl-1,3,2-dioxaborolan-2-yl)aniline (0.379 g, 1.356 mmol), 4-bromo-N,N-dimethylpyridin-2-amine (0.3 g, 1.49 mmol) and potassium carbonate (0.6 g, 4.34 mmol) were suspended in a mixture of dioxane (10 mL) and water (1 mL). After degassing with nitrogen for 15 minutes, Pd(dppf)Cl₂. CH₂Cl₂ (0.055 g, 0.068 mmol) was added and the mixture was heated to 75° C. for 1 hour. The mixture was cooled to room temperature, and diluted with EtOAc (10 mL) and water (5 mL). The organic phase was separated, dried (MgSO₄), filtered and concentrated in vacuo to give a brown oil. The crude product was purified by chromatography on silica (24 g column, 0-60% EtOAc/isohexane) to afford the title compound (201 mg, 49%) as an orange oil.

¹H NMR (CDCl₃) δ 8.27 (d, J=5.6 Hz, 1H), 6.96 (dd, J=9.9, 3.0 Hz, 1H), 6.79-6.72 (m, 2H), 6.69 (s, 1H), 3.70 (s, 2H), 3.26 (s, 6H), 2.94 (sept, J=7.0 Hz, 1H), 1.31 (d, J=6.8 Hz, 6H).

LCMS m/z 274.4 (M+H)⁺ (ES⁺); 272.8 (M−H)⁻ (ES⁻).

### Intermediate A33: 4-Fluoro-2-isopropyl-6-(2-(prop-1-yn-1-yl)pyridin-4-yl)aniline

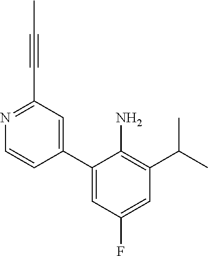 [SMILES: CC#Cc1cc(-c2cc(F)cc(C(C)C)c2N)ccn1]

The title compound was prepared according to the procedure for 4-(2-amino-5-fluoro-3-isopropylphenyl)-N,N-dimethylpyridin-2-amine (Intermediate A32) (218 mg, 57%).

¹H NMR (CDCl₃) δ 8.63 (d, J=5.3 Hz, 1H), 7.56 (s, 1H), 7.41 (d, J=5.3 Hz, 1H), 6.97 (dd, J=9.9, 2.9 Hz, 1H), 6.72 (dd, J=8.5, 3.0 Hz, 1H), 4.30-2.50 (br s, 2H), 2.93 (sept, J=6.6 Hz, 1H), 2.14 (s, 3H), 1.31 (d, J=6.8 Hz, 6H).

LCMS m/z 269.3 (M+H)⁺ (ES⁺); 267.2 (M−H)⁻ (ES⁻).

### Intermediate A34: 7-Fluoro-5-(2-methoxypyridin-4-yl)-2,3-dihydro-1H-inden-4-amine

### Step A: N-(7-Fluoro-2,3-dihydro-1H-inden-4-yl)pivalamide

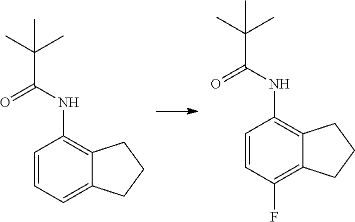 [SMILES: CC(C)(C)C(=O)Nc1cccc2c1CCC2 ; CC(C)(C)C(=O)Nc1ccc(F)c2c1CCC2]

To an ice-cooled solution of N-(2,3-dihydro-1H-inden-4-yl)pivalamide (2.5 g, 11.50 mmol) in dry dichloromethane (50 mL) was added pyridine hydrofluoride (9 ml, 69.9 mmol). The pale yellow mixture was stirred for 30 minutes at 0° C. A solution of bis(tert-butylcarbonyloxy)iodobenzene (7.5 g, 17.91 mmol) in dichloromethane (10 mL) was then slowly added over 10 minutes to the mixture. The reaction was slowly allowed to reach room temperature and stirred overnight. It was then quenched with triethylamine (0.5 ml, 3.58 mmol) and the whole mixture was absorbed onto silica gel and purified by chromatography on silica gel (120 g column, 0-30% EtOAc/isohexane) to afford the title compound (0.635 g, 22%) as a yellow crystalline solid.

¹H NMR (CDCl₃) δ 7.68 (dd, J=8.8, 4.5 Hz, 1H), 7.14 (s, 1H), 6.87 (t, J=8.6 Hz, 1H), 3.01 (t, J=7.5 Hz, 2H), 2.85 (t, J=7.5 Hz, 2H), 2.18 (p, J=7.5 Hz, 2H), 1.34 (s, 9H).

LCMS m/z 236.3 (M+H)⁺ (ES⁺); 234.2 (M−H)⁻ (ES⁻).

### Step B: 7-Fluoro-2,3-dihydro-1H-inden-4-amine

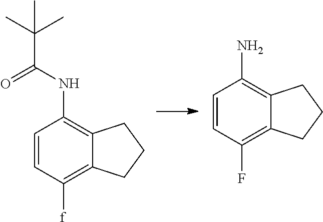

N-(7-Fluoro-2,3-dihydro-1H-inden-4-yl)pivalamide (0.632 g, 2.69 mmol) was dissolved in ethanol (5 mL) and stirred at room temperature. H₂SO₄ (95% aq.) (5 ml, 89 mmol) was slowly added to water (5 mL) and this mixture was then added to the reaction mixture. The slurry was heated to 100° C. (bath temperature) over the weekend. The reaction mixture was cooled to room temperature, diluted with water (10 mL) and then basified with 2M aq. NaOH. The mixture was extracted with dichloromethane (3×100 mL). The combined organics were washed, dried by passing through a hydrophobic frit and concentrated in vacua. The crude product was purified by chromatography on silica gel (24 g column, 0-30% EtOAc/isohexane) to afford the title compound (350 mg, 82%) as a pale pink oil that solidified on standing. ¹H NMR (CDCl₃) δ 6.71 (dd, J=9.0, 8.2 Hz, 1H), 6.46 (dd, J=8.5, 3.9 Hz, 1H), 3.45 (s, 2H), 2.96 (t, J=7.6 Hz, 2H), 2.77 (t, J=7.5 Hz, 2H), 2.16 (p, J=7.6 Hz, 2H).

LCMS m/z 152.3 (M+H)⁺ (ES⁺).

### Step C: 5-Bromo-7-fluoro-2,3-dihydro-1H-inden-4-amine

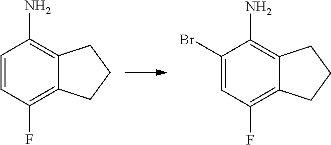 [SMILES: Nc1ccc(F)c2c1CCC2 ; Nc1c(Br)cc(F)c2c1CCC2]

7-Fluoro-2,3-dihydro-1H-inden-4-amine (345 mg, 2.282 mmol) was dissolved in dichloromethane (10 mL). NBS (450 mg, 2.53 mmol) was added at room temperature in a single portion. The mixture turned dark brown immediately and was stirred for 15 minutes at room temperature. The reaction mixture was partitioned between dichloromethane and 1M aq. NaOH (20 mL) and stirred for 15 minutes. The organic phase was separated and washed with brine (10 mL), and then dried by passing through a hydrophobic frit. The solvent was removed in vacua to give a dark brown oil. The crude product was purified by chromatography on silica gel (24 g column, 0-20% EtOAc/isohexane) to afford the title compound (323 mg, 55%) as a dark purple oil.

¹H NMR (CDCl₃) δ 7.08 (d, J=7.8 Hz, 1H), 3.06 (t, J=7.5 Hz, 2H), 2.95 (t, J=7.5 Hz, 2H), 2.20 (p, J=7.6 Hz, 2H), NH₂ not observed.

### Step D: 7-Fluoro-5-(2-methoxypyridin-4-yl)-2,3-dihydro-1H-inden-4-amine

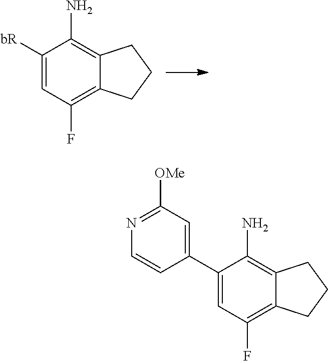

5-Bromo-7-fluoro-2,3-dihydro-1H-inden-4-amine (320 mg, 1.391 mmol) was dissolved in dioxane (5 mL). A solution of potassium carbonate (600 mg, 4.34 mmol) in water (1 mL) and solid (2-methoxypyridin-4-yl)boronic acid (250 mg, 1.635 mmol) were added. The mixture was degassed with nitrogen for 15 minutes before Pd(dppf)Cl₂.CH₂Cl₂ (60 mg, 0.073 mmol) was added. The reaction mixture was heated to 80° C. (bath temperature) for 24 hours. The mixture was cooled to room temperature and partitioned between dichloromethane (30 mL) and water (20 mL). The organic phase was dried by passing through a hydrophobic frit and concentrated in vacua to give a brown oil. The crude product was purified by chromatography on silica gel (12 g column, 0-50% EtOAc/isohexane) to afford the title compound (0.185 g, 49%) as a pale brown oil that crystallized on standing.

¹H NMR (CDCl₃) δ 8.27 (d, J=5.4 Hz, 1H), 7.06 (d, J=5.3 Hz, 1H), 6.95 (s, 1H), 6.73 (d, J=9.0 Hz, 1H), 4.03 (s, 3H), 3.00 (t, J=7.5 Hz, 2H), 2.85 (t, J=7.4 Hz, 2H), 2.23 (p, J=7.5 Hz, 2H), NH₂ not observed.

LCMS m/z 259.3 (M+H)⁺ (ES⁺).

### Intermediate A35: 5-(2-Methoxypyridin-4-yl)-2,3-dihydro-1H-inden-4-amine

### Step A: N-(5-Bromo-2,3-dihydro-1H-inden-4-yl)pivalamide

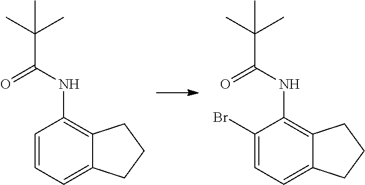 [SMILES: CC(C)(C)C(=O)Nc1cccc2c1CCC2 ; CC(C)(C)C(=O)Nc1c(Br)ccc2c1CCC2]

N-(2,3-Dihydro-1H-inden-4-yl)pivalamide (1 g, 4.60 mmol), p-toluenesulfonic acid monohydrate (0.45 g, 2.366 mmol), Pd(OAc)₂ (0.05 g, 0.223 mmol), and NBS (0.9 g, 5.06 mmol) were suspended in toluene (20 mL) and stirred under air for 16 hours. The dark green mixture was diluted with EtOAc (20 mL), and then washed with saturated aq. NaHCO₃ (2×10 mL), water (2×10 mL) and brine (10 mL). The organic phase was dried (Na₂SO₄), filtered and concentrated in vacua to give a dark green amorphous solid. The crude product was purified by chromatography on silica gel (40 g column, 0-30% EtOAc/isohexane) to afford the title compound (1.662 g, 100%) as a colourless crystalline solid that was contaminated with a small amount of reaction byproducts.

LCMS m/z 296.3/298.3 (M+H)⁺ (ES⁺).

### Step B: 5-Bromo-2,3-dihydro-1H-inden-4-amine

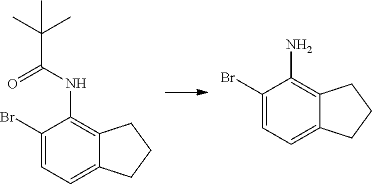 [SMILES: CC(C)(C)C(=O)Nc1c(Br)ccc2c1CCC2 ; Nc1c(Br)ccc2c1CCC2]

N-(5-Bromo-2,3-dihydro-1H-inden-4-yl)pivalamide (0.632 g, 2.134 mmol) was dissolved in ethanol (5 mL) and stirred at room temperature. H₂SO₄ (95% aq.) (5 ml, 89 mmol) was slowly added to water (5 mL) and this mixture was then added to the reaction mixture. The slurry was heated to 100° C. (bath temperature) at which point the mixture became homogeneous and it was stirred at this temperature over the weekend. The mixture was cooled to room temperature and then basified with 2M aq. NaOH. The mixture was extracted with dichloromethane (3×20 mL). The organic phase was dried by passing through a hydrophobic frit, and then concentrated in vacuo. The crude product was purified by chromatography on silica gel (40 g column, 0-50% EtOAc/isohexane) to afford the title compound (0.138 g, 29%).

¹H NMR (CDCl₃) δ 7.23 (d, J=7.9 Hz, 1H), 6.57 (d, J=8.0 Hz, 1H), 3.92 (s, 2H), 2.89 (t, J=7.6 Hz, 2H), 2.77 (t, J=7.4 Hz, 2H), 2.15 (p, J=7.5 Hz, 2H).

### Step C: 5-(2-Methoxypyridin-4-yl)-2,3-dihydro-1H-inden-4-amine

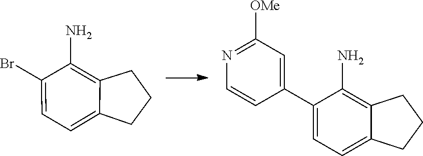 [SMILES: Nc1c(Br)ccc2c1CCC2 ; COc1cc(-c2ccc3c(c2N)CCC3)ccn1]

5-Bromo-2,3-dihydro-1H-inden-4-amine (280 mg, 1.320 mmol) was dissolved in dioxane (5 mL). A solution of potassium carbonate (boo mg, 4.34 mmol) in water (1 mL) and (2-methoxypyridin-4-yl)boronic acid (250 mg, 1.635 mmol) were added. The mixture was degassed with nitrogen for 15 minutes before Pd(dppf)Cl₂. CH₂Cl₂ (60 mg, 0.073 mmol) was added. The reaction mixture was heated to 80° C. (bath temperature) for 2 hours. The mixture was cooled to room temperature and partitioned between dichloromethane (30 mL) and water (20 mL). The organic phase was dried by passing through a hydrophobic frit and concentrated in vacua to give a brown oil. The crude product was purified by chromatography on silica gel (12 g column, 0-50% EtOAc/isohexane) to afford the title compound (0.289 g, 87%) as a pale yellow crystalline solid.

¹H NMR (CDCl₃) δ 8.26 (d, J=5.4 Hz, 1H), 7.11 (d, J=5.0 Hz, 1H), 7.01 (d, J=7.7 Hz, 1H), 6.97 (s, 1H), 6.80 (d, J=7.6 Hz, 1H), 4.06 (s, 3H), 2.98 (t, J=7.6 Hz, 2H), 2.80 (t, J=7.4 Hz, 2H), 2.19 (p, J=7.5 Hz, 2H), NH₂ not observed.

LCMS m/z 241.3 (M+H)⁺ (ES⁺).

### Intermediate A36: 4-(4-Amino-2,3-dihydro-1H-inden-5-yl)picolinonitrile

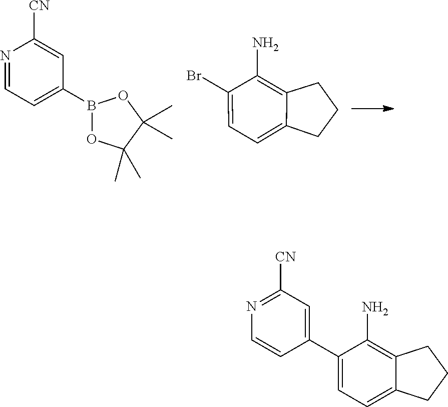

Prepared according to the general procedure of 5-(2-methoxypyridin-4-yl)-2,3-dihydro-1H-inden-4-amine (Intermediate A35, Step C) from 5-bromo-2,3-dihydro-1H-inden-4-amine (Intermediate A35, Step B) and 4-(4,4,5,5-tetramethyl-1,3,2-dioxaborolan-2-yl)picolinonitrile to afford the title compound (215 mg, 61%) as a pale yellow solid.

1H (DMSO-d6) δ 8.72 (dd, J=5.1, 0.8 Hz, 1H), 8.03 (dd, J=1.8, 0.8 Hz, 1H), 7.74 (dd, J=5.1, 1.8 Hz, 1H), 6.91 (d, J=7.7 Hz, 1H), 6.61 (d, J=7.7 Hz, 1H), 4.94 (s, 2H), 2.83 (t, J=7.4 Hz, 2H), 2.71 (t, J=7.4 Hz, 2H), 2.03 (p, J=7.4 Hz, 2H).

LCMS: m/z 236.3 (M+H)⁺ (ES⁺).

### Intermediate A37: 4-(5-Fluoro-2-isocyanato-3-isopropylphenyl)picolinonitrile

### Step A: 4-Fluoro-2-(prop-1-en-2-yl)aniline

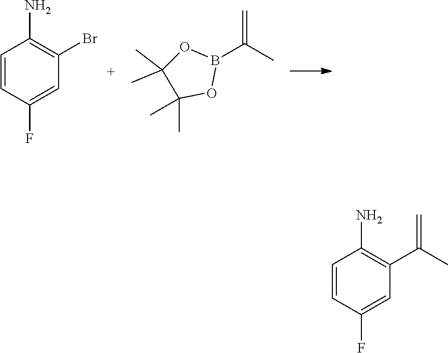

To a mixture of 2-bromo-4-fluoroaniline (39 g, 205.25 mmol, 1 eq), 4,4,5,5-tetramethyl-2-(prop-1-en-2-yl)-1,3,2-dioxaborolane (36.21 g, 215.51 mmol, 1.05 eq) and K₂CO₃ (70.92 g, 513.12 mmol, 2.5 eq) in dioxane (200 mL) and H₂O (40 mL) was added Pd(dppf)Cl₂ (7.51 g, 10.26 mmol, 0.05 eq) under a nitrogen atmosphere. Then the reaction mixture was stirred at 80° C. for 5 hours. The reaction mixture was quenched by addition of H₂O (600 mL) and extracted with EtOAc (2×500 mL). The combined organic layers were washed with brine (2×600 mL), dried over anhydrous Na₂SO₄, filtered and concentrated under reduced pressure. The residue was purified by silica gel column chromatography (SiO₂, petroleum ether:ethyl acetate 1:0 to 100:1) to give the title compound (27 g, 77% yield, 89% purity on LCMS) as a yellow oil.

¹H NMR (CDCl₃) δ 6.81-6.76 (m, 2H), 6.66-6.62 (m, 1H), 5.38 (s, 1H), 5.08 (s, 1H), 3.69 (br s, 2H) and 1.25 (s, 3H).

LCMS: m/z 152.2 (M+H)⁺ (ES⁺).

### Step B: 4-Fluoro-2-isopropylaniline

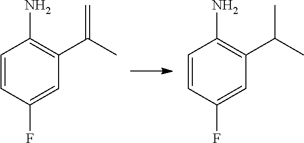

To a solution of 4-fluoro-2-(prop-1-en-2-yl)aniline (21 g, 138.91 mmol, 1 eq) in MeOH (300 mL) was added Pd/C (2.1 g, 178.59 mmol, 10 wt % loading on activated carbon) under a nitrogen atmosphere. The reaction mixture was degassed in vacua and purged with hydrogen several times. The reaction mixture was stirred at 25° C. for 12 hours under hydrogen (50 psi). The reaction mixture was filtered and the filtrate was concentrated in vacuo to give the title compound (20 g, crude) as a yellow oil.

¹H NMR (CDCl₃) δ 6.86 (dd, 1H), 6.75-6.72 (m, 1H), 6.63-6.61 (m, 1H), 3.50 (br s, 2H), 2.95-2.84 (m, 1H) and 1.25 (d, 6H).

LCMS: m/z 154.2 (M+H)⁺ (ES⁺).

### Step C: 2-Bromo-4-fluoro-6-isopropylaniline

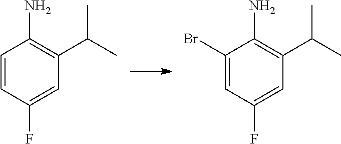

To a solution of 4-fluoro-2-isopropylaniline (20 g, 130.55 mmol, 1 eq) in toluene (250 mL) was added NBS (23.24 g, 130.55 mmol, 1 eq) at 25° C. The reaction mixture was stirred at 25° C. for 10 minutes. The reaction mixture was poured into H₂O (300 mL) and extracted with EtOAc (2×250 mL). The combined organic phases were washed with brine (2×400 mL), dried over anhydrous Na₂SO₄, filtered and concentrated in vacua. The residue was purified by silica gel column chromatography (SiO₂, eluting only by using petroleum ether) to give the title compound (30 g, 99%) as a black brown oil.

¹H NMR (CDCl₃) δ 6.99 (dd, 1H), 6.78 (dd, 1H), 3.91 (br s, 2H), 2.88-2.71 (m, 1H) and 1.17 (d, 6H).

LCMS: m/z 232.1 (M+H)⁺ (ES⁺).

### Step D: 4-(2-Amino-5-fluoro-3-isopropylphenyl)picolinonitrile

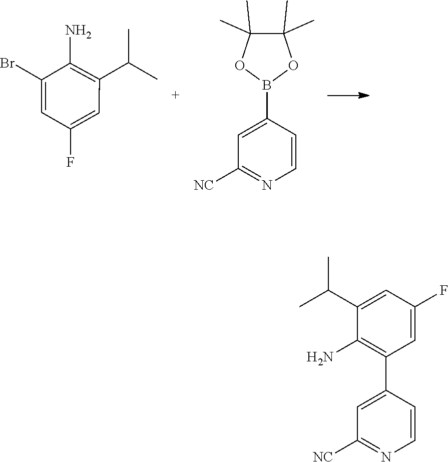 [SMILES: CC(C)c1cc(F)cc(Br)c1N ; CC1(C)OB(c2ccnc(C#N)c2)OC1(C)C ; CC(C)c1cc(F)cc(-c2ccnc(C#N)c2)c1N]

To a solution of 2-bromo-4-fluoro-6-isopropylaniline (3.6 g, 15.51 mmol, 1 eq) and 4-(4,4,5,5-tetramethyl-1,3,2-dioxaborolan-2-yl)picolinonitrile (3.60 g, 15.67 mmol, 1.01 eq) in dioxane (90 mL) and H₂O (9 mL) was added Na₂CO₃ (4.11 g, 38.78 mmol, 2.5 eq). Then Pd(dppf)Cl₂ (1.13 g, 1.55 mmol, 0.1 eq) was added to the mixture under a nitrogen atmosphere. The resulting mixture was stirred at 80° C. for 2 hours under nitrogen. Then the mixture was concentrated in vacua. The residue was purified by silica gel column chromatography (SiO₂, petroleum ether:ethyl acetate, 20:1 to 5:1) and then triturated with petroleum ether (10 mL) to give the title compound (2.65 g, 65% yield, 97% purity on LCMS) as a yellow solid.

1HNMR (CDCl₃) δ 8.79 (d, 1H), 7.86 (d, 1H), 7.65 (dd, 1H), 6.99 (dd, 1H), 6.70 (dd, 1H), 3.63 (br s, 2H), 2.98-2.87 (m, 1H) and 1.30 (d, 6H).

LCMS: m/z 256.2 (M+H)⁺ (ES⁺).

### Step E: 4-(5-Fluoro-2-isocyanato-3-isopropylphenyl)picolinonitrile

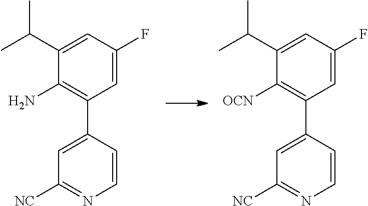

To a solution of 4-(2-amino-5-fluoro-3-isopropylphenyl)picolinonitrile (1 g, 3.92 mmol, 1 eq) in THF (40 mL) was added TEA (793 mg, 7.83 mmol, 2 eq). To the above mixture was added triphosgene (465 mg, 1.57 mmol, 0.4 eq) in portions at 5° C. Then the mixture was stirred at 70° C. for 1 hour. The mixture was diluted with EtOAc (200 mL) and then filtered through silica gel. The filtrate was concentrated in vacuo to give the title compound (1.2 g, crude) as a yellow solid, which was used directly in the next step.

### Intermediate A38: 4-(5-Fluoro-2-isocyanato-3-isopropylphenyl)-2-methoxypyridine

### Step A: 4-Fluoro-2-isopropyl-6-(2-methoxypyridin-4-yl)aniline

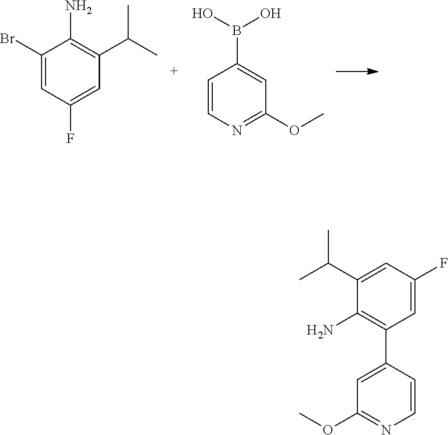 [SMILES: CC(C)c1cc(F)cc(Br)c1N ; COc1cc(B(O)O)ccn1 ; COc1cc(-c2cc(F)cc(C(C)C)c2N)ccn1]

To a solution of 2-bromo-4-fluoro-6-isopropylaniline (12 g, 51.70 mmol, 1 eq) in dioxane (240 mL) and H₂O (48 mL) was added (2-methoxypyridin-4-yl)boronic acid (9.49 g, 62.04 mmol, 1.2 eq) and Na₂CO₃ (13.70 g, 129.26 mmol, 2.5 eq). The reaction mixture was purged with nitrogen three times. Then Pd(dppf)Cl₂ (3.78 g, 5.17 mmol, 0.1 eq) was added to the mixture under a nitrogen atmosphere. The resulting mixture was heated at 80° C. for 2 hours. The reaction mixture was quenched with H₂O (800 mL) and extracted with EtOAc (2×600 mL). The combined organic layers were washed with brine (2×800 mL), dried over anhydrous Na₂SO₄, filtered and concentrated under reduced pressure. The residue was purified by silica gel column chromatography (SiO₂, petroleum ether:ethyl acetate, 70:1 to 10:1) and then triturated with hexane (100 mL) to give the title compound (10.05 g, 72% yield, 96% purity on LCMS).

¹H NMR (CDCl₃) δ 8.24 (d, 1H), 6.97 (d, 1H), 6.93 (d, 1H), 6.83 (s, 1H), 6.73-6.70 (m, 1H), 3.99 (s, 3H), 3.66 (br s, 2H), 2.97-2.89 (m, 1H) and 1.29 (dd, 6H).

LCMS: m/z 261.1 (M+H)⁺ (ES⁺).

### Step B: 4-(5-Fluoro-2-isocyanato-3-isopropylphenyl)-2-methoxypyridine

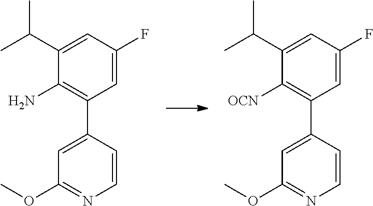

To a solution of 4-fluoro-2-isopropyl-6-(2-methoxypyridin-4-yl)aniline (1 g, 3.84 mmol, 1 eq) in THF (40 mL) was added TEA (777 mg, 7.68 mmol, 2 eq). Then triphosgene (456 mg, 1.54 mmol, 0.4 eq) was added in portions at 5° C. The mixture was stirred at 70° C. for 1 hour. The mixture was diluted with EtOAc (200 mL) and filtered through silica gel. The filtrate was concentrated in vacuo to give the title compound (1.1 g, crude) as a yellow oil, which was used directly in the next step.

### Intermediate A39: 4-(4-Isocyanato-2,3-dihydro-1H-inden-5-yl)-2-methoxypyridine

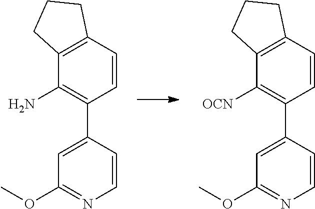

To a solution of 5-(2-methoxypyridin-4-yl)-2,3-dihydro-1H-inden-4-amine (Intermediate A35) (11 g, 45.78 mmol, 1 eq) and TEA (5.10 g, 50.35 mmol, 1.1 eq) in THF (275 mL) was added in portions bis(trichloromethyl) carbonate (4.93 g, 16.61 mmol, 0.36 eq) at 0° C. Then the reaction mixture was stirred at 16° C. for 0.5 hour. The reaction mixture was filtered and the filter cake was washed with THF (2 L). The filtrate was concentrated in vacua to give the title compound (9.04 g, 74%) as a light yellow solid.

¹H NMR (CDCl₃) δ 8.28 (d, 1H), 7.20-7.16 (m, 3H), 7.02 (s, 1H), 4.16 (s, 3H), 3.04-2.99 (m, 4H) and 2.23-2.15 (m, 2H).

### Intermediate A40: 4-(7-Fluoro-4-isocyanato-2,3-dihydro-1H-inden-5-yl)pyridine

### Step A: 7-Fluoro-4-nitro-2,3-dihydro-1H-inden-1-one

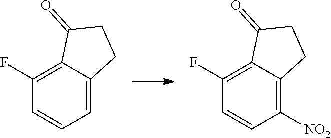 [SMILES: O=C1CCc2cccc(F)c21 ; O=C1CCc2c([N+](=O)[O-])ccc(F)c21]

To a mixture of 7-fluoro-2,3-dihydro-1H-inden-1-one (9.5 g, 63.27 mmol, 1 eq) in concentrated H₂SO₄ (100 mL) was added dropwise a solution of HNO₃ (5.37 mL, 82.25 mmol, 69 wt % in water, 1.3 eq) in concentrated H₂SO₄ (20 mL) at −15° C. Then the reaction mixture was stirred at 0° C. for 0.5 hour. The mixture was quenched with water (500 mL) at 0° C., and then extracted with EtOAc (3×300 mL). The combined organic phases were dried over anhydrous Na₂SO₄, filtered and concentrated in vacua. The residue was purified by silica gel column chromatography (SiO₂, petroleum ether:ethyl acetate, 10:1 to 3:1) to give the title compound (11.4 g, 92%) as a yellow solid.

¹H NMR (CDCl₃) δ 8.51 (dd, 1H), 7.22 (t, 1H), 3.69-3.65 (m, 2H) and 2.88-2.82 (m, 2H).

### Step B: 7-Fluoro-4-nitro-2,3-dihydro-1H-inden-1-ol

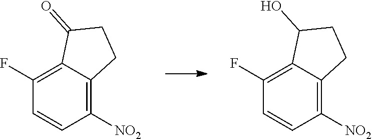 [SMILES: O=C1CCc2c([N+](=O)[O-])ccc(F)c21 ; O=[N+]([O-])c1ccc(F)c2c1CCC2O]

To a mixture of 7-fluoro-4-nitro-2,3-dihydro-1H-inden-1-one (30 g, 153.73 mmol, 1 eq) in EtOH (450 mL) was added NaBH₄ (11.63 g, 307.46 mmol, 2 eq) in portions. The reaction mixture was stirred at 15° C. for 1 hour. Then the mixture was poured into water (500 mL) and extracted with DCM (2×200 mL). The combined organic phases were washed with brine (200 mL), dried over anhydrous Na₂SO₄, filtered and concentrated in vacuo to give the title compound (30 g, crude) as brown oil.

¹H NMR (CDCl₃) δ 8.21 (dd, 1H), 7.08 (t, 1H), 5.59-5.56 (m, 1H), 3.66-3.59 (m, 1H), 3.44-3.39 (m, 1H), 2.56-2.51 (m, 1H) and 2.22-2.17 (m, 2H).

### Step C: 4-Fluoro-7-nitro-2,3-dihydro-1H-indene

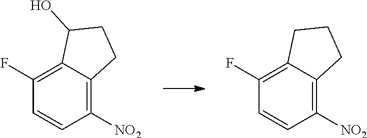 [SMILES: O=[N+]([O-])c1ccc(F)c2c1CCC2O ; O=[N+]([O-])c1ccc(F)c2c1CCC2]

To a mixture of 7-fluoro-4-nitro-2,3-dihydro-1H-inden-1-ol (4.5 g, 22.82 mmol, 1 eq) in TFA (20 mL) was added Et₃SiH (7.96 g, 68.47 mmol, 3 eq) in one portion. The reaction mixture was stirred at 25° C. for 12 hours. Then the mixture was quenched with water (100 mL) and extracted with EtOAc (3×100 mL). The combined organic layers were washed with saturated aqueous NaHCO₃ solution (2×100 mL), dried over anhydrous Na₂SO₄, filtered and concentrated in vacua to give the title compound (5 g, crude) as brown oil.

¹H NMR (CDCl₃) δ 8.06 (dd, 1H), 7.01 (t, 1H), 3.46 (t, 2H), 3.04 (t, 2H) and 2.25-2.20 (m, 2H).

### Step D: 7-Fluoro-2,3-dihydro-1H-inden-4-amine

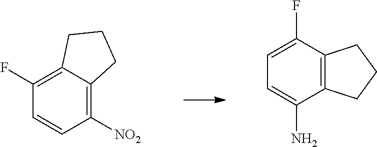 [SMILES: O=[N+]([O-])c1ccc(F)c2c1CCC2 ; Nc1ccc(F)c2c1CCC2]

To a mixture of 4-fluoro-7-nitro-2,3-dihydro-1H-indene (5 g, 27.60 mmol, 1 eq) in MeOH (50 mL) was added Pd/C (0.5 g, 10 wt % loading on activated carbon) at 25° C. under a nitrogen atmosphere. Then the reaction mixture was stirred at 25° C. for 12 hours under hydrogen (15 psi). The mixture was filtered and the filtrate was concentrated in vacuo. The residue was purified by silica gel column chromatography (SiO₂, petroleum ether:ethyl acetate, 50:1 to 10:1) to give the title compound (1.8 g, 43%) as a brown solid.

¹H NMR (CDCl₃) δ 6.69 (t, 1H), 6.44 (dd, 1H), 3.47 (br s, 2H), 2.95 (t, 2H), 2.75 (t, 2H) and 2.19-2.11 (m, 2H).

### Step E: 5-Bromo-7-fluoro-2,3-dihydro-1H-inden-4-amine

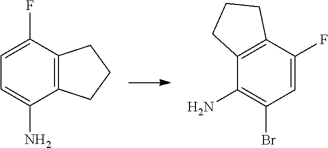 [SMILES: Nc1ccc(F)c2c1CCC2 ; Nc1c(Br)cc(F)c2c1CCC2]

To a solution of 7-fluoro-2,3-dihydro-1H-inden-4-amine (8.3 g, 54.90 mmol, 1 eq) in toluene (100 mL) was added NBS (10.26 g, 57.65 mmol, 1.05 eq) in one portion at 25° C. The reaction mixture turned dark brown immediately and then the mixture was stirred at 25° C. for 30 minutes. The reaction mixture was quenched with saturated aqueous Na₂SO₃ solution (200 mL) and extracted with EtOAc (2×100 mL). The combined organic phases were washed with brine (100 mL), dried over anhydrous Na₂SO₄, filtered and concentrated in vacua. The residue was purified by silica gel column chromatography (SiO₂, petroleum ether:ethyl acetate, 1:0 to 20:1) to give the title compound (8.51 g, 67%) as a brown solid.

¹H NMR (CDCl₃) δ 6.99 (d, 1H), 3.81 (br s, 2H), 2.92 (t, 2H), 2.78 (t, 2H) and 2.21-2.13 (m, 2H).

### Step F: 7-Fluoro-5-(pyridin-4-yl)-2,3-dihydro-1H-inden-4-amine

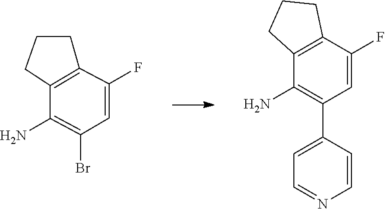 [SMILES: Nc1c(Br)cc(F)c2c1CCC2 ; Nc1c(-c2ccncc2)cc(F)c2c1CCC2]

To a mixture of 5-bromo-7-fluoro-2,3-dihydro-1H-inden-4-amine (3.5 g, 15.21 mmol, 1 eq) and pyridin-4-ylboronic acid (1.96 g, 15.97 mmol, 1.05 eq) in dioxane (50 mL) and H₂O (5 mL) was added K₂CO₃ (6.31 g, 45.64 mmol, 3 eq) and Pd(dppf)Cl₂ (1.11 g, 1.52 mmol, 0.1 eq) in one portion under a nitrogen atmosphere. Then the reaction mixture was heated to 80° C. for 12 hours. The reaction mixture was filtered. The filtrate was diluted with water (50 mL) and extracted with EtOAc (3×100 mL). The combined organic phases were washed with brine (100 mL), dried over anhydrous Na₂SO₄, filtered and concentrated in vacua. The residue was purified by silica gel column chromatography (SiO₂, petroleum ether:ethyl acetate, 10:1 to 2:1) to give the title compound (1.7 g, 45% yield, 90.98% purity on HPLC) as a brown solid.

¹H NMR (CDCl₃) δ 8.68 (dd, 2H), 7.40 (dd, 2H), 6.72 (d, 1H), 3.76 (br s, 2H), 3.01 (t, 2H), 2.80 (t, 2H) and 2.26-2.18 (m, 2H).

### Step G: 4-(7-Fluoro-4-isocyanato-2,3-dihydro-1H-inden-5-yl)pyridine

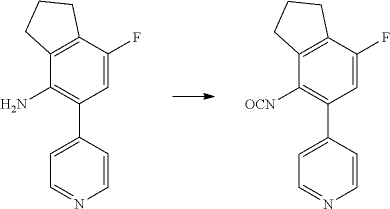

To a solution of 7-fluoro-5-(pyridin-4-yl)-2,3-dihydro-1H-inden-4-amine (400 mg, 1.75 mmol, 1 eq) and TEA (355 mg, 3.50 mmol, 2 eq) in THF (30 mL) was added bis(trichloromethyl) carbonate (208 mg, 700.94 μmol, 0.4 eq) at 0° C. The reaction mixture was stirred at 70° C. for 30 minutes. Then the reaction mixture was filtered through a pad of silica gel and the filter cake was washed with THF (20 mL). The filtrate was concentrated in vacuo to reduce to 10 mL, which was used directly in the next step.

### Intermediate A41: 3-(5-Fluoro-2-isocyanato-3-isopropylphenyl)pyridine

### Step A: 4-Fluoro-2-isopropyl-6-(pyridin-3-yl)aniline

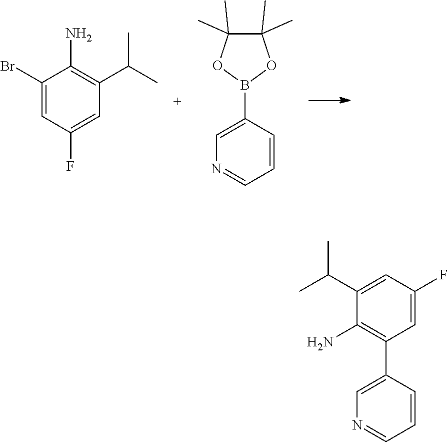

To a solution of 2-bromo-4-fluoro-6-isopropylaniline (21 g, 90.48 mmol, 1 eq) in dioxane (450 mL) and H₂O (90 mL) was added 3-(4,4,5,5-tetramethyl-1,3,2-dioxaborolan-2-yl)pyridine (22.26 g, 108.58 mmol, 1.2 eq) and Na₂CO₃ (23.98 g, 226.20 mmol, 2.5 eq). The reaction mixture was purged with nitrogen three times. Then Pd(dppf)Cl₂ (5.10 g, 6.97 mmol, 0.077 eq) was added under a nitrogen atmosphere. The resulting mixture was heated to 80° C. and stirred for 2 hours. The reaction mixture was quenched by addition of H₂O (800 mL) and extracted with EtOAc (2×600 mL). The combined organic layers were washed with brine (2×800 mL), dried over anhydrous Na₂SO₄, filtered and concentrated under reduced pressure. The residue was purified by silica gel column chromatography (SiO₂, petroleum ether:ethyl acetate, 50:1 to 1:1) and then triturated with hexane (40 mL) to give the title compound (17 g, 82%) as a grey solid.

¹H NMR (CDCl₃) δ 8.70 (d, 1H), 8.63 (dd, 1H), 7.79 (dd, 1H), 7.41-7.38 (m, 1H), 6.94 (dd, 1H), 6.71 (dd, 1H), 3.57 (s, 2H), 2.97-2.88 (m, 1H) and 1.30 (d, 6H).

LCMS: m/z 231.2 (M+H)⁺ (ES⁺).

### Step B: 3-(5-Fluoro-2-isocyanato-3-isopropylphenyl)pyridine

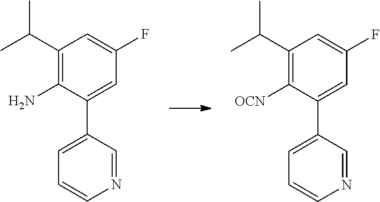

To a solution of 4-fluoro-2-isopropyl-6-(pyridin-3-yl)aniline (0.5 g, 2.17 mmol, 1 eq) and TEA (439 mg, 4.34 mmol, 2 eq) in THF (10 mL) was added triphosgene (257 mg, 868.51 μmol, 0.4 eq) in portions at 5° C. Then the reaction mixture was heated to 70° C. and stirred for 1 hour. The reaction mixture was concentrated in vacua. The residue was treated with EtOAc (100 mL) and filtered. The filtrate was concentrated in vacuo to give the title compound (0.2 g, crude) as a yellow oil, which was used directly in the next step.

### Intermediate A42: 4-(7-Fluoro-4-isocyanato-2,3-dihydro-1H-inden-5-yl)-2-methoxypyridine

### Step A: 7-Fluoro-5-(2-methoxypyridin-4-yl)-2,3-dihydro-1H-inden-4-amine

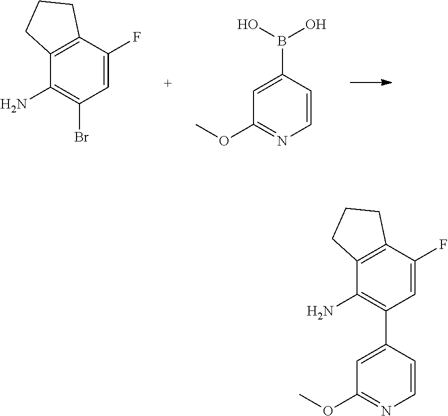 [SMILES: Nc1c(Br)cc(F)c2c1CCC2 ; COc1cc(B(O)O)ccn1 ; COc1cc(-c2cc(F)c3c(c2N)CCC3)ccn1]

To a mixture of 5-bromo-7-fluoro-2,3-dihydro-1H-inden-4-amine (Intermediate A4.0, Step E) (8.5 g, 36.94 mmol, 1 eq) and (2-methoxypyridin-4-yl)boronic acid (5.93 g, 38.79 mmol, 1.05 eq) in dioxane (150 mL) and water (15 mL) were added K₂CO₃ (15.32 g, 110.83 mmol, 3 eq) and Pd(dppf)Cl₂ (2.70 g, 3.69 mmol, 0.1 eq) in one portion under nitrogen. Then the reaction mixture was heated to 80° C. and stirred for 12 hours. The reaction mixture was quenched with water (300 mL) and extracted with EtOAc (3×300 mL). The combined organic layers were washed with brine (100 mL), dried with anhydrous Na₂SO₄, filtered and concentrated in vacua. The residue was purified by silica gel chromatography (petroleum ether:EtOAc, 1:0 to 10:1) and then purified by trituration with a mixture of TBME and n-hexane (50 mL, 1:20) to give the title compound (5.06 g, 52% yield, 97.44% purity on LCMS) as an off-white solid.

¹H NMR (CDCl₃) δ 8.23 (d, 1H), 6.99 (dd, 1H), 6.86 (s, 1H), 6.71 (d, 1H), 3.99 (s, 3H), 3.67 (br s, 2H), 3.00 (t, 2H), 2.79 (t, 2H) and 2.25-2.17 (m, 2H).

### Step B: 4-(7-Fluoro-4-isocyanato-2,3-dihydro-1H-inden-5-yl)-2-methoxypyridine

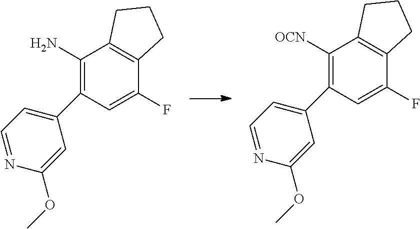

To a solution of phosgene (1.5 mL, 20 wt % in toluene, 2.9 mmol) in toluene (40 mL) was added dropwise a solution of 7-fluoro-5-(2-methoxypyridin-4-yl)-2,3-dihydro-1H-inden-4-amine (300 mg, 1.16 mmol) in toluene (20 mL) at ambient temperature. The resulting reaction mixture was then heated to reflux for 70 minutes and upon cooling was concentrated in vacua to afford the title compound as a brown oil (325 mg, 98%). The crude product was used directly in the next step without further purification.

¹H NMR (CDCl₃) δ 8.24 (d, 1H), 6.95 (dd, 1H), 6.88 (s, 1H), 6.85-6.75 (m, 1H), 4.00 (s, 3H), 3.15-2.95 (m, 4H), 2.32-2.12 (m, 2H).

### Intermediate A43: 4-(4-Isocyanato-2,3-dihydro-1H-inden-5-yl)picolinonitrile

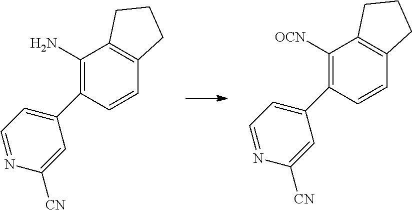

To a solution of phosgene (1.7 mL, 20 wt % in toluene, 3.2 mmol) in toluene (40 mL) was added dropwise a solution of 4-(4-amino-2,3-dihydro-1H-inden-5-yl)picolinonitrile (Intermediate A36) (300 mg, 1.3 mmol) in toluene (20 mL) at ambient temperature. The resulting reaction mixture was then heated to reflux for 70 minutes and upon cooling was concentrated in vacua to afford the title compound as a brown oil (333 mg, 100%). The crude product was used directly in the next step without further purification.

¹H NMR (CDCl₃) δ 8.75 (dd, 1H), 7.81 (dd, 1H), 7.63 (dd, 1H), 7.22-7.08 (m, 2H), 3.04 (m, 4H), 2.23 (m, 2H).

### Intermediate A44: 4-(4-Isocyanato-2,3-dihydro-1H-inden-5-yl)pyridine

### Step A: 5-(Pyridin-4-yl)-2,3-dihydro-1H-inden-4-amine

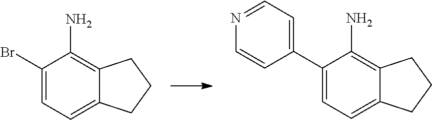 [SMILES: Nc1c(Br)ccc2c1CCC2 ; Nc1c(-c2ccncc2)ccc2c1CCC2]

5-Bromo-2,3-dihydro-1H-inden-4-amine (1.2 g, 5.7 mmol) was dissolved in dioxane (25 mL). A solution of potassium carbonate (3.1 g, 23 mmol) in water (6 mL) and pyridin-4-ylboronic acid (0.83 g, 6.8 mmol) were added. The mixture was degassed with nitrogen for 20 minutes before Pd(dppf)Cl₂.DCM (0.74 g, 0.91 mmol) was added. The reaction mixture was heated to 77° C. for 2 hours. Then the mixture was cooled to room temperature and filtered over Celite with DCM (100 mL) and water (25 mL). The organic phase was dried (Na₂SO₄), filtered and concentrated in vacuo to give a brown oil (3.3 g). The crude product was purified by chromatography on silica gel (80 g column, 0-100% EtOAc/heptane) to afford the title compound (0.75 g, 63%) as a pale yellow crystalline solid.

¹H NMR (CDCl₃) δ 8.72-8.54 (m, 2H), 7.50-7.37 (m, 2H), 6.97 (d, 1H), 6.78 (d, 1H), 3.72 (s, 2H), 2.96 (t, 2H), 2.77 (t, 2H), 2.18 (m, 2H).

### Step B: 4-(4-Isocyanato-2,3-dihydro-1H-inden-5-yl)pyridine

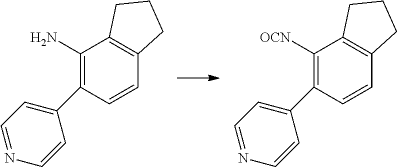

To a solution of phosgene (1.1 mL, 20 wt % in toluene, 2.06 mmol) in toluene (40 mL) was added dropwise a solution of 5-(pyridin-4-yl)-2,3-dihydro-1H-inden-4-amine (175 mg, 0.83 mmol) in toluene (20 mL) at ambient temperature. The resulting reaction mixture was then heated to reflux for 70 minutes and upon cooling to room temperature a yellow precipitate was formed. The solid was filtered and dried in vacua to afford the title compound as a yellow solid (145 mg, 74%). The crude product was used directly in the next step without further purification.

¹H NMR (CDCl₃) δ 8.76 (d, 2H), 8.04 (d, 2H), 7.26-7.08 (m, 2H), 3.08 (t, 4H), 2.26 (m, 2H).

### Intermediate A45: 4-(6-Isocyanato-2,3-dihydro-1H-inden-5-yl)-2-methoxypyridine

### Step A: 6-Bromo-2,3-dihydro-1H-inden-5-amine

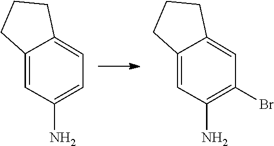 [SMILES: Nc1ccc2c(c1)CCC2 ; Nc1cc2c(cc1Br)CCC2]

To a solution of 2,3-dihydro-1H-inden-5-amine (10.6 g, 79.59 mmol, 1 eq) in toluene (150 mL) was added NBS (17.00 g, 95.50 mmol, 1.2 eq) in portions, and then the mixture was stirred at 25° C. for 12 hours. The reaction mixture was quenched with saturated aqueous Na₂SO₃ solution (100 mL) and then extracted with EtOAc (3×150 mL). The combined organic layers were dried over Na₂SO₄, filtered and concentrated in vacua. The residue was purified by silica gel column chromatography (SiO₂, petroleum ether: ethyl acetate, 1:0 to 20:1) to give the title compound (9.5 g, 56%) as a brown solid.

¹H NMR (CDCl₃): δ 7.15 (s, 1H), 6.56 (s, 1H), 3.72 (br s, 2H), 2.70-2.61 (m, 4H) and 1.95-1.85 (m, 2H).

### Step B: 6-(2-Methoxypyridin-4-yl)-2,3-dihydro-1H-inden-5-amine

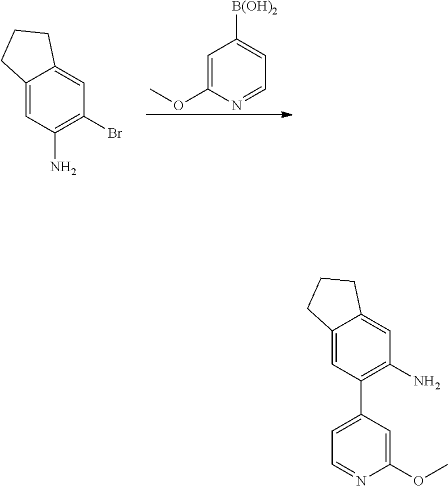 [SMILES: Nc1cc2c(cc1Br)CCC2 ; COc1cc(B(O)O)ccn1 ; COc1cc(-c2cc3c(cc2N)CCC3)ccn1]

To a solution of 6-bromo-2,3-dihydro-1H-inden-5-amine (1 g, 4.72 mmol, 1 eq) and (2-methoxypyridin-4-yl)boronic acid (793 mg, 5.19 mmol, 1.1 eq) in dioxane (15 mL) and H₂O (2 mL) was added K₂CO₃ (1.95 g, 14.15 mmol, 3 eq) and Pd(dppf)Cl₂ (345 mg, 471.51 μmol, 0.1 eq) in one portion under N₂. Then the reaction mixture was heated to 80° C. and stirred for 2 hours. The reaction mixture was washed with water (20 mL) and extracted with EtOAc (3×20 mL). The combined organic layers were washed with brine (20 mL), dried over Na₂SO₄, filtered and concentrated in vacua. The residue was purified by silica gel column chromatography (SiO₂, petroleum ether:ethyl acetate, 15:1 to 10:1) to give the title compound (556.4 mg, 49%) as a yellow solid.

¹H NMR (CDCl₃): δ 8.24 (d, 1H), 7.05 (d, 1H), 7.03 (s, 1H), 6.85 (s, 1H), 6.71 (s, 1H), 3.96 (s, 3H), 2.92-2.76 (m, 4H) and 2.15-2.05 (m, 2H).

### Step C: 4-(6-Isocyanato-2,3-dihydro-1H-inden-5-yl)-2-methoxypyridine

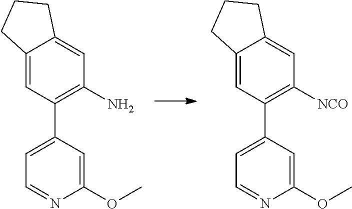

To a solution of 6-(2-methoxypyridin-4-yl)-2,3-dihydro-1H-inden-5-amine (200 mg, 832.29 μmol, 1 eq) and TEA (168 mg, 1.66 mmol, 2 eq) in THF (2 mL) was added triphosgene (99 g, 332.92 μmol, 0.4 eq) at 0° C. Then the reaction mixture was heated to 70° C. for 1 hour. The reaction mixture was filtered by silica gel and washed with THF (50 mL). Then the filtrate was concentrated in vacua to give the title compound (246 mg, crude) as a light yellow solid, which was used directly in the next step.

### Intermediate A46: 4-(5-Fluoro-2-isocyanato-3-isopropylphenyl)-2-isopropoxypyridine

### Step A: 4-Fluoro-2-(prop-1-en-2-yl)aniline

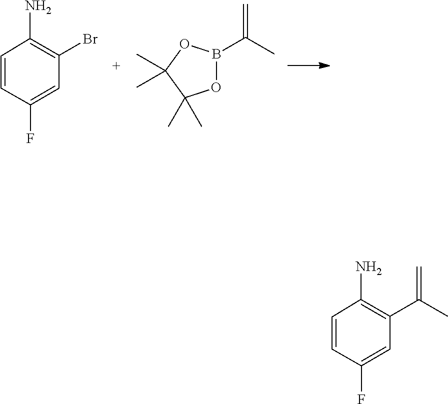

To a mixture of 2-bromo-4-fluoroaniline (39 g, 205.25 mmol, 1 eq), 4,4,5,5-tetramethyl-2-(prop-1-en-2-yl)-1,3,2-dioxaborolane (36.21 g, 215.51 mmol, 1.05 eq) and K₂CO₃ (70.92 g, 513.12 mmol, 2.5 eq) in dioxane (200 mL) and H₂O (40 mL) was added Pd(dppf)Cl₂ (7.51 g, 10.26 mmol, 0.05 eq) under N₂ atmosphere. Then the reaction mixture was stirred at 80° C. for 5 hours. The reaction mixture was quenched by addition of H₂O (600 mL) and extracted with EtOAc (2×500 mL). The combined organic layers were washed with brine (2×600 mL), dried over anhydrous Na₂SO₄, filtered and concentrated under reduced pressure. The residue was purified by silica gel column chromatography (petroleum ether:ethyl acetate=1:0 to 100:1) to give the title compound (27 g, 77% yield, 89% purity on LCMS) as a yellow oil.

¹H NMR (CDCl₃): δ 6.81-6.76 (m, 2H), 6.66-6.62 (m, 1H), 5.38 (s, 1H), 5.08 (s, 1H), 3.69 (br s, 2H) and 1.25 (s, 3H).

LCMS: m/z 152.2 (M+H)⁺ (ES⁺).

### Step B: 4-Fluoro-2-isopropylaniline

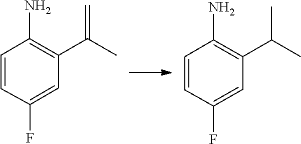

To a solution of 4-fluoro-2-(prop-1-en-2-yl)aniline (21 g, 138.91 mmol, 1 eq) in MeOH (300 mL) was added Pd/C (2.1 g, 178.59 mmol, 10 wt % loading on activated carbon) under N₂ atmosphere. The reaction mixture was degassed in vacuo and purged with H2 several times. The reaction mixture was stirred at 25° C. for 12 hours under H2 (50 psi). The reaction mixture was filtered and the filtrate was concentrated in vacuo to give the title compound (20 g, crude) as a yellow oil.

¹H NMR (CDCl₃): δ 6.86 (dd, 1H), 6.75-6.72 (m, 1H), 6.63-6.61 (m, 1H), 3.50 (br s, 2H), 2.95-2.84 (m, 1H) and 1.25 (d, 6H).

LCMS: m/z 154.2 (M+H)⁺ (ES⁺).

### Step C: 2-Bromo-4-fluoro-6-isopropylaniline

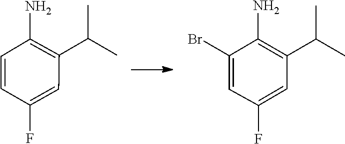

To a solution of 4-fluoro-2-isopropylaniline (20 g, 130.55 mmol, 1 eq) in toluene (250 mL) was added NBS (23.24 g, 130.55 mmol, 1 eq) at 25° C. The reaction mixture was stirred at 25° C. for 10 minutes. Then the reaction mixture was poured into H₂O (300 mL) and extracted with EtOAc (2×250 mL). The organic phases were washed with brine (2×400 mL), dried over anhydrous Na₂SO₄, filtered and concentrated in vacua. The residue was purified by silica gel column chromatography (eluting only by using petroleum ether) to give the title compound (30 g, 99%) as a black brown oil.

¹H NMR (CDCl₃): δ 6.99 (dd, 1H), 6.78 (dd, 1H), 3.91 (br s, 2H), 2.88-2.71 (m, 1H) and 1.17 (d, 6H).

LCMS: m/z 232.1 (M+H)⁺ (ES^{+).}

### Step D: 4-Bromo-2-isopropoxypyridine

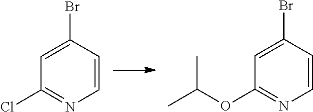

To a solution of 4-bromo-2-chloropyridine (20 g, 103.93 mmol, 1 eq) in THF (400 mL) was added NaH (6.24 g, 155.89 mmol, 60% purity, 1.5 eq) at 0° C. Then the mixture was stirred for 0.5 hour. Propan-2-ol (6.87 g, 114.32 mmol, 8.75 mL, 1.1 eq) was added and the resulting mixture was warmed to 50° C. and stirred for 12 hours. The reaction mixture was quenched with H₂O (1 L) at 25° C. and extracted with EtOAc (2×200 mL). The combined organic layers were washed with brine (200 mL), dried over Na₂SO₄, filtered and concentrated under reduced pressure. The residue was purified by column chromatography (SiO₂, petroleum ether:ethyl acetate=50:1 to 40:1) to give the title compound (22 g, 98%) as a light yellow oil.

¹H NMR (CDCl₃): δ 7.96 (d, 1H), 6.98 (dd, 1H), 6.89 (d, 1H), 5.44-5.24 (m, 1H) and 1.34 (d, 6H).

### Step E: 2-Isopropoxy-4-(4,4,5,5-tetramethyl-1,3,2-dioxaborolan-2-yl)pyridine

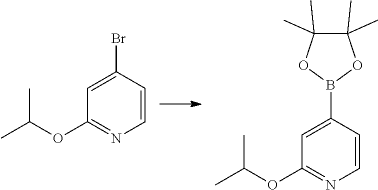 [SMILES: CC(C)Oc1cc(Br)ccn1 ; CC(C)Oc1cc(B2OC(C)(C)C(C)(C)O2)ccn1]

To a solution of 4-bromo-2-isopropoxypyridine (19 g, 87.93 mmol, 1 eq) and 4,4,4′,4′,5,5,5′,5′-octamethyl-2,2′-bi(1,3,2-dioxaborolane) (22.33 g, 87.93 mmol, 1 eq) in 1,4-dioxane (300 mL) was added KOAc (25.89 g, 263.80 mmol, 3 eq) followed by Pd(dppf)Cl₂ (1.93 g, 2.64 mmol, 0.03 eq) under nitrogen. Then the reaction mixture was heated to 80° C. and stirred for 12 hours. The mixture was concentrated in vacuo.

The residue was purified by silica gel column chromatography (SiO₂, petroleum ether:ethyl acetate=50:1 to 20:1) to give the title compound (22 g, 95%) as a light yellow oil.

¹H NMR (CDCl₃): δ 8.16 (d, 1H), 7.13 (d, 1H), 7.08 (s, 1H), 5.32-5.24 (m, 1H), 1.34 (s, 12H) and 1.27 (s, 6H).

LCMS: m/z 264.2 (M+H)⁺ (ES⁺).

### Step F: 4-Fluoro-2-(2-isopropoxypyridin-4-yl)-6-isopropylaniline

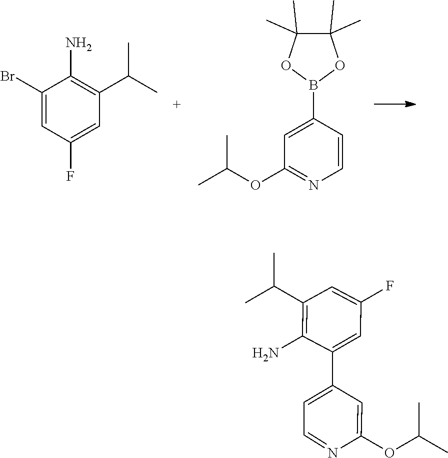 [SMILES: CC(C)c1cc(F)cc(Br)c1N ; CC(C)Oc1cc(B2OC(C)(C)C(C)(C)O2)ccn1 ; CC(C)Oc1cc(-c2cc(F)cc(C(C)C)c2N)ccn1]

To a solution of 2-bromo-4-fluoro-6-isopropylaniline (10.94 g, 47.12 mmol, 1 eq) and 2-isopropoxy-4-(4,4,5,5-tetramethyl-1,3,2-dioxaborolan-2-yl)pyridine (12.4 g, 47.12 mmol, 1 eq) in 1,4-dioxane (200 mL) and H₂O (20 mL) was added Pd(dppf)Cl₂ (1.72 g, 2.36 mmol, 0.05 eq) followed by K₂CO₃ (19.54 g, 141.37 mmol, 3 eq) at 25° C. Then the reaction mixture was heated to 80° C. and stirred for 2 hours. The mixture was filtered and the filtrate was concentrated in vacua. The residue was purified by silica gel column chromatography (SiO₂, petroleum ether:ethyl acetate=50:1 to 20:1) to give the title compound (10.3 g, 69% yield, 91% purity on LCMS) as a brown oil.

¹H NMR (CDCl₃): δ 8.21 (d, 1H), 6.94-6.91 (m, 2H), 6.76 (s, 1H), 6.72 (dd, 1H), 5.38-5.29 (m, 1H), 3.64 (br s, 2H), 2.98-2.89 (m, 1H), 1.38 (d, 6H) and 1.30-1.27 (m, 6H).

LCMS: m/z 289.2 (M+H)⁺ (ES⁺).

### Step G: 4-(5-Fluoro-2-isocyanato-3-isopropylphenyl)-2-isopropoxypyridine

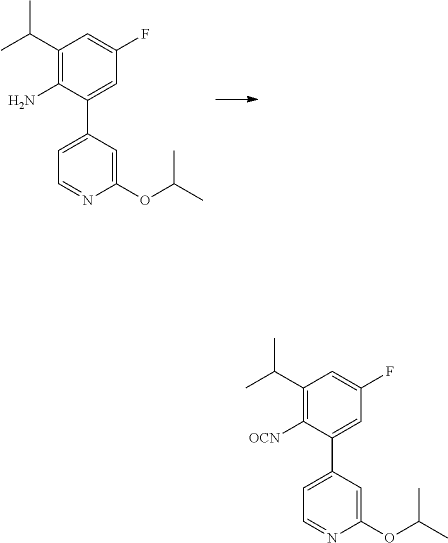

To a solution of 4-fluoro-2-(2-isopropoxypyridin-4-yl)-6-isopropylaniline (4 g, 13.87 mmol, 1 eq) in THF (80 mL) was added TEA (2.81 g, 27.74 mmol, 3.86 mL, 2 eq). The mixture was cooled to 0° C. and then triphosgene (1.65 g, 5.55 mmol, 0.4 eq) was added to the mixture. The resulting mixture was heated to 70° C. and stirred for 1 hour. The mixture was filtered and the filtrate was concentrated in vacuo. The residue was purified by silica gel column chromatography (SiO₂, petroleum ether:ethyl acetate=100:1 to 30:1) to give the title compound (1.9 g, 44% yield) as a yellow oil, which was used directly in the next step.

### Intermediate A47: 7-Cyclopropyl-5-(2-methoxypyridin-4-yl)-2,3-dihydro-1H-inden-4-amine

### Step A: 7-Bromo-5-(2-methoxypyridin-4-yl)-2,3-dihydro-1H-inden-4-amine

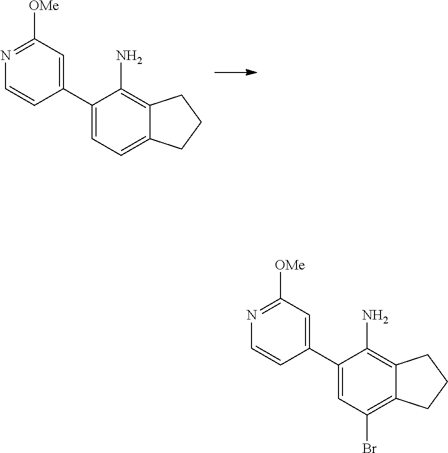 [SMILES: COc1cc(-c2ccc3c(c2N)CCC3)ccn1 ; COc1cc(-c2cc(Br)c3c(c2N)CCC3)ccn1]

NBS (389 mg, 2.185 mmol) was added to a mixture of 5-(2-methoxypyridin-4-yl)-2,3-dihydro-1H-inden-4-amine (Intermediate A35) (500 mg, 2.081 mmol) in CHCl₃ (5 ml) with cooling in an ice bath. The resultant solution was stirred at room temperature for 16 hours, washed with 10% sodium thiosulfate solution (20 ml), brine (10 ml), dried over MgSO₄ and concentrated in vacua. The crude product was purified by chromatography on silica gel (40 g cartridge, 0-30% EtOAc/isohexane) to afford the title compound (400 mg, 57%) as a tan solid.

¹H NMR (DMSO-d6) δ 8.20 (d, J=5.3 Hz, 1H), 7.04-6.97 (m, 2H), 6.80 (d, J=1.3 Hz, 1H), 4.84 (s, 2H), 3.89 (s, 3H), 2.83 (q, J=7.1 Hz, 4H), 2.06 (p, J=7.6 Hz, 2H).

LCMS; m/z 318.9/320.9 (M+H)+ (ES+).

### Step B: 7-Cyclopropyl-5-(2-methoxypyridin-4-yl)-2,3-dihydro-1H-inden-4-amine

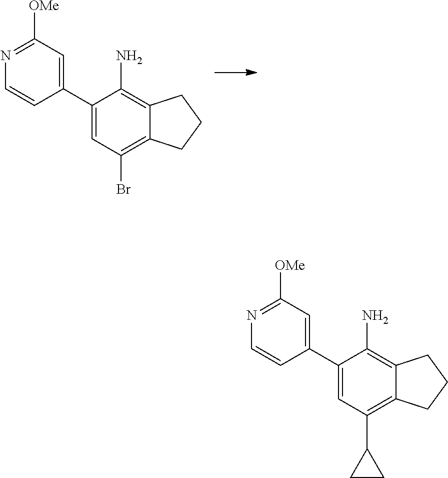 [SMILES: COc1cc(-c2cc(Br)c3c(c2N)CCC3)ccn1 ; COc1cc(-c2cc(C3CC3)c3c(c2N)CCC3)ccn1]

A stirred mixture of 7-bromo-5-(2-methoxypyridin-4-yl)-2,3-dihydro-1H-inden-4-amine (100 mg, 0.313 mmol), K₂CO₃ (87 mg, 0.627 mmol), tricyclohexylphosphine (11.42 mg, 0.041 mmol), and cyclopropylboronic acid (29.6 mg, 0.345 mmol) in toluene (10 ml) and water (2 ml) at room temperature was degassed with nitrogen for 15 minutes. After this time palladium (II) acetate (7.03 mg, 0.031 mmol) was added and the reaction mixture was left to stir at 90° C. for 24 hours. The reaction mixture was cooled and concentrated in vacua. The crude product was purified by chromatography on silica gel (12 g cartridge, 0-30% EtOAc/isohexane) to afford the title compound (56 mg, 54%) as a colourless solid on standing.

¹H NMR (DMSO-d6) δ 8.17 (d, J=5.2 Hz, 1H), 7.00 (dd, J=5.3, 1.5 Hz, 1H), 6.78 (d, J=1.4 Hz, 1H), 6.43 (s, 1H), 4.48 (s, 2H), 3.88 (s, 3H), 2.91 (t, J=7.5 Hz, 2H), 2.72 (t, J=7.4 Hz, 2H), 2.04 (q, =7.3 Hz, 2H), 1.78-1.71 (m, 1H), 0.81-0.75 (m, 2H), 0.55-0.48 (m, 2H).

LCMS; m/z 281.5 (M+H)⁺ (ES⁺).

### Intermediate A48: 4-(4-Isocyanato-2,3-dihydro-1H-inden-5-yl)-2-methoxypyridine

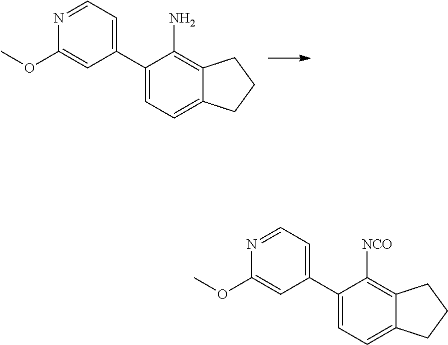

5-(2-Methoxypyridin-4-yl)-2,3-dihydro-1H-inden-4-amine (Intermediate A35) (500 mg, 2.081 mmol) was dissolved in DCM (10 to mL) and sat aq NaHCO₃ (5 mL) was added. A solution of triphosgene (250 mg, 0.842 mmol) in DCM (5 mL) was added and the mixture stirred at room temperature for 1 hour. The organic phase was separated, dried by passing through a hydrophobic frit and concentrated in vacuo to afford the title compound (523 mg, 94%) as a pale yellow oil that was used without further purification.

¹H NMR (CDCl₃) δ 8.25 (d, J=5.2 Hz, 1H), 7.18-7.13 (m, 2H), 7.01 (dd, J=5.3, 1.5 Hz, 1H), 6.86 (s, 1H), 4.03 (s, 3H), 3.04 (t, J=7.5 Hz, 4H), 2.21 (p, J=7.5 Hz, 2H).

### Intermediate A49: 4-(4-Isocyanato-2,3-dihydrobenzofuran-5-yl)-2-methoxypyridine

### Step A: N-(5-Bromo-2,3-dihydrobenzofuran-4-yl)acetamide

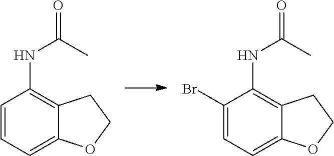

N-(2,3-dihydrobenzofuran-4-yl)acetamide (13.1 g, 73.9 mmol), 4-methylbenzenesulfonic acid hydrate (7.73 g, 4.07 mmol) and diacetoxypalladium (0.830 g, 3.70 mmol) were suspended in toluene (250 mL) and stirred for 20 minutes. NBS (14.47 g, 81 mmol) was added and the mixture was stirred for 30 minutes, diluted with EtOAc (150 mL), and washed with aq NaHCO₃ (100 mL) and aq Na₂S₂O₃ (10 wt %, 100 mL). The aqueous phases were further extracted with DCM (150 mL). The organic phases were combined, dried (MgSO₄), filtered and concentrated under reduced pressure to afford the title compound (22.27 g, quant., purity 85% by LCMS) which was used crude in the next step.

LCMS; m/z 255.9, 257.9 (M+H)⁺ (ES⁺).

### Step B: 5-Bromo-2,3-dihydrobenzofuran-4-amine

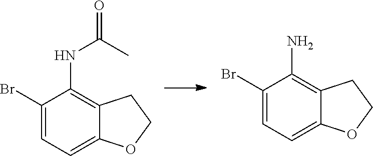

A solution of N-(5-bromo-2,3-dihydrobenzofuran-4-yl)acetamide (22.27 g, 73.9 mmol) in MeOH (400 mL) and cone H₂SO₄ (40 mL) was stirred at reflux for 18 hours. The volatiles were removed under reduced pressure, the residue taken up in DCM (300 mL) and basified with aq NaOH 1 M (100 mL). The organic phase was separated, dried (Na₂SO₄), filtered and concentrated under reduced pressure. The crude product was purified by chromatography on silica gel (220 g cartridge, 0-100% EtOAc/isohexane) to afford the title compound (9.17 g, 57%) as an off white solid.

¹H NMR (CDCl₃) δ 7.16 (dt, J=8.4, 0.9 Hz, 1H), 6.17 (d, J=8.4 Hz, 1H), 4.61 (t, J=8.7 Hz, 2H), 3.99 (br. s, 2H), 3.05 (t, J=8.7 Hz, 2H).

### Step C: 5-(2-Methoxypyridin-4-yl)-2,3-dihydrobenzofuran-4-amine

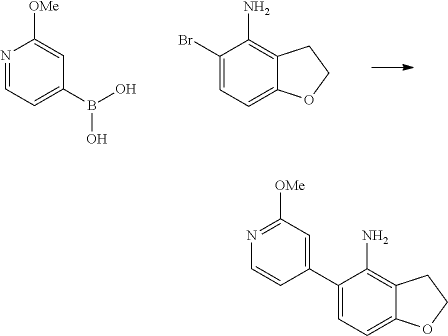 [SMILES: COc1cc(B(O)O)ccn1 ; Nc1c(Br)ccc2c1CCO2 ; COc1cc(-c2ccc3c(c2N)CCO3)ccn1]

Prepared according to the general procedure of 5-(2-methoxypyridin-4-yl)-2,3-dihydro-1H-inden-4-amine (Intermediate A35) from 5-bromo-2,3-dihydrobenzofuran-4-amine and (2-methoxypyridin-4-yl)boronic acid to afford the title compound (2.25 g, 79%) as an off white solid.

¹H NMR (DMSO-d₆) δ 8.15 (d, J=5.2 Hz, 1H), 6.99 (dd, J=5.3, 1.5 Hz, 1H), 6.84 (d, J=8.2 Hz, 1H), 6.78 (s, 1H), 6.14 (d, J=8.1 Hz, 1H), 4.91 (s, 2H), 4.54 (t, J=8.7 Hz, 2H), 3.87 (s, 3H), 3.01 (t, J=8.7 Hz, 2H).

LCMS; m/z 243.1 (M+H)⁺ (ES⁺).

### Step D: 4-(4-Isocyanato-2,3-dihydrobenzofuran-5-yl)-2-methoxypyridine

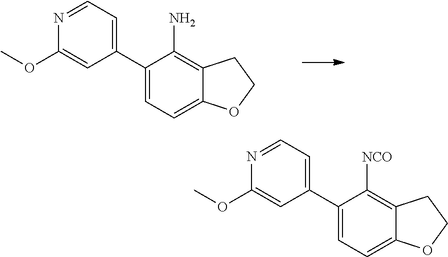

Prepared according to the general procedure of 4-(4-isocyanato-2,3-dihydro-1H-inden-5-yl)-2-methoxypyridine (Intermediate A48) from 5-(2-methoxypyridin-4-yl)-2,3-dihydrobenzofuran-4-amine to afford the title compound (926 mg, 79%) as a pale yellow solid.

¹H NMR (CDCl₃) δ 8.23 (d, J=5.3 Hz, 1H), 7.13 (d, J=8.3 Hz, 1H), 6.98 (dd, J=5.3, 1.4 Hz, 1H), 6.83 (s, 1H), 6.74 (d, J=8.3 Hz, 1H), 4.72 (t, J=8.7 Hz, 2H), 4.02 (s, 3H), 3.33 (t, J=8.7 Hz, 2H).

### PREPARATION OF EXAMPLES

### Example 1: N-((4-Fluoro-2-iso-propyl-6-(pyridin-3-yl)phenyl)carbamoyl)-1-iso-propyl-1H-pyrazole-3-sulfonamide

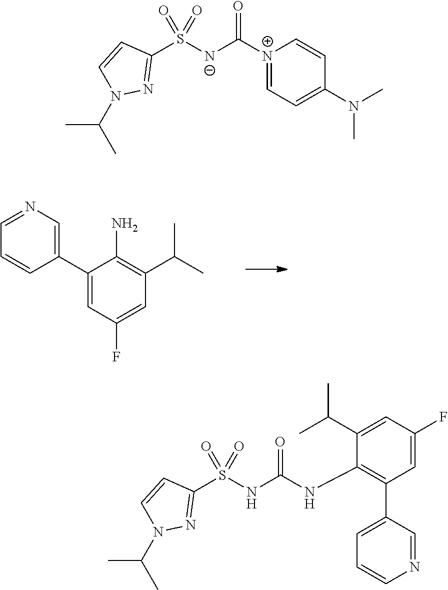

4-Fluoro-2-isopropyl-6-(pyridin-3-yl)aniline (Intermediate A1; 50 mg, 0.213 mmol) in acetonitrile (2 mL) was added to (4-(dimethylamino)pyridin-1-ium-1-carbonyl)((1-isopropyl-1H-pyrazol-3-yl)sulfonyl)amide (Intermediate P3; 71.8 mg, 0.213 mmol) and the mixture was stirred at 50° C. for 10 minutes*, then at room temperature for 2 hours. The reaction mixture was purified by preparative HPLC (basic method, 10-40% acetonitrile in 10 mM aqueous ammonium bicarbonate, 6.5 minute run) to afford the title compound (41 mg, 42%) as a white solid.

(*The reaction was usually performed for between 10 minutes and 1 hour heating.)

¹H NMR (DMSO-d₆) δ 11.34 (s, 1H), 8.96 (dd, 1H), 8.93 (d, 1H), 8.35 (d, 1H), 8.29 (s, 1H), 8.14 (dt, 1H), 7.78 (dd, 1H), 7.62 (dd, 1H), 7.48 (dd, 1H), 7.05-6.85 (m, 1H), 5.02 (sept, 1H), 3.48-3.34 (m, 1H), 1.86 (d, 6H) and 1.51 (d, 6H).

LCMS m/z 446.4 (M+H)⁺ (ES⁺); 444.3 (M−H)⁻ (ES⁻).

The following examples 2-35 were synthesised following the general procedure for N-((4-fluoro-2-iso-propyl-6-(pyridin-3-yl)phenyl)carbamoyl)-1-iso-propyl-1H-pyrazole-3-sulfonamide (Example 1) above. Sodium salts were synthesised using sodium tert-butoxide where stated.

### Example 2: N-((4-Fluoro-2-iso-propyl-6-(1-methyl-1H-pyrazol-4-yl)phenyl) carbamoyl)-1-iso-propyl-1H-pyrazole-3-sulfonamide

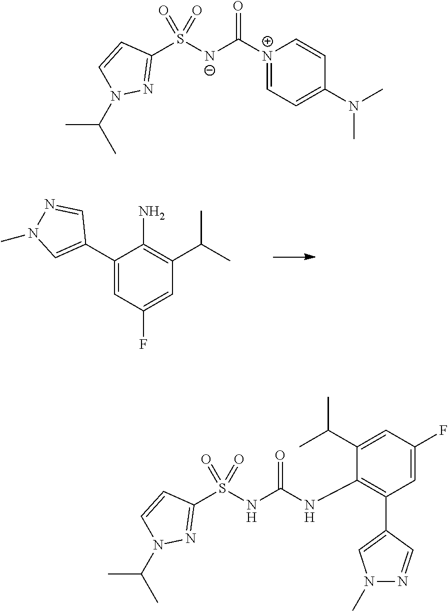

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(1-methyl-1H-pyrazol-4-yl)aniline (Intermediate A2) to afford the title compound (50 mg, 53%). ¹H NMR (DMSO-d₆) δ 11.07 (br s, 1H), 7.95 (d, 1H), 7.93 (s, 1H), 7.89 (s, 1H), 7.64 (br s, 1H), 7.14 (dd, 1H), 6.99 (dd, 1H), 6.65 (d, 1H), 4.60 (sept, 1H), 3.85 (s, 3H), 3.02-2.88 (m, 1H), 1.43 (d, 6H) and 1.06 (d, 6H).

LCMS m/z 449.4 (M+H)⁺ (ES⁺).

### Example 3: N-((4-Fluoro-2-iso-propyl-6-(1-methyl-1H-imidazol-5-yl) phenyl)carbamoyl)-1-iso-propyl-1H-pyrazole-3-sulfonamide

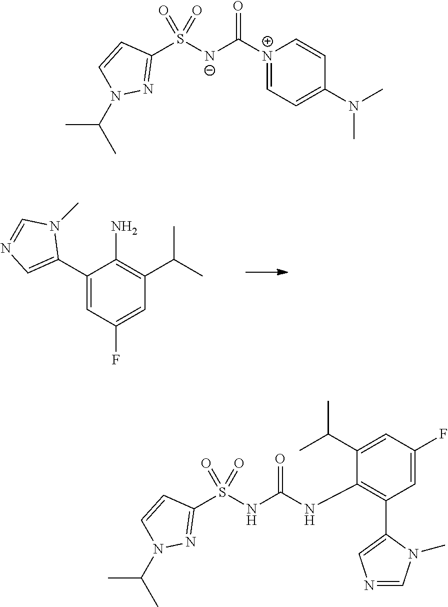

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(1-methyl-1H-imidazol-5-yl)aniline (Intermediate A3) to afford the title compound (20.1 mg, 42%) as an off-white solid.

¹H NMR (DMSO-d₆) δ 10.96 (s, 1H), 7.92 (s, 1H), 7.65 (s, 2H), 7.18 (dd, 1H), 7.04 (dd, 1H), 6.77 (s, 1H), 6.53 (s, 1H), 4.61 (sept, 1H), 3.40 (s, 3H), 3.06-2.87 (m, 1H), 1.45 (d, 6H) and 1.08 (d, 6H).

LCMS m/z 449.4 (M+H)⁺ (ES⁺); 447.1 (M−H)⁻ (ES⁻).

### Example 4: N-((5-Fluoro-3-iso-propyl-[1,1′-biphenyl]-2-yl)carbamoyl)-1-iso-propyl-1H-pyrazole-3-sulfonamide

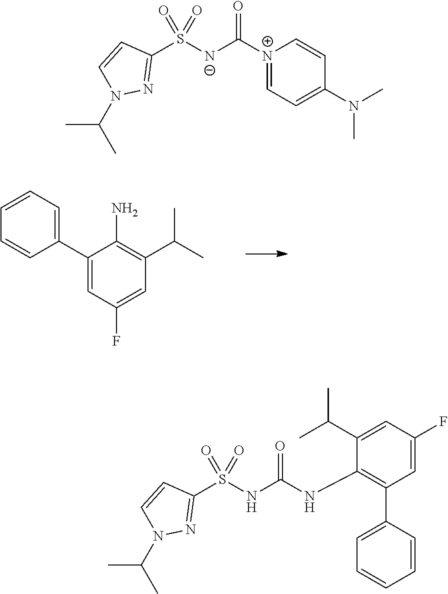

Prepared from (4-(dimethylamino)pyridin-1-ium-1-carbonyl)((1-isopropyl-1H-pyrazol-3-yl)sulfonyl)amide (Intermediate P3) and 5-fluoro-3-isopropyl-[1,1′-biphenyl]-2-amine (Intermediate A4) to afford the title compound (26 mg, 38%) as a white solid.

¹H NMR (DMSO-d₆) δ 10.79 (br s, 1H), 7.97 (d, 1H), 7.68 (s, 1H), 7.43-7.21 (m, 5H), 7.15 (dd, 1H), 6.96 (dd, 1H), 6.57 (d, 1H), 4.60 (sept, 1H), 3.02-2.87 (m, 1H), 1.44 (d, 6H) and 1.08 (d, 6H).

LCMS m/z 445.4 (M+H)⁺ (ES⁺); 443.4 (M−H)⁻ (ES⁻).

### Example 5: N-((4-Fluoro-2-iso-propyl-6-(1-methyl-1H-pyrazol-5-yl)phenyl) carbamoyl)-1-iso-propyl-1H-pyrazole-3-sulfonamide

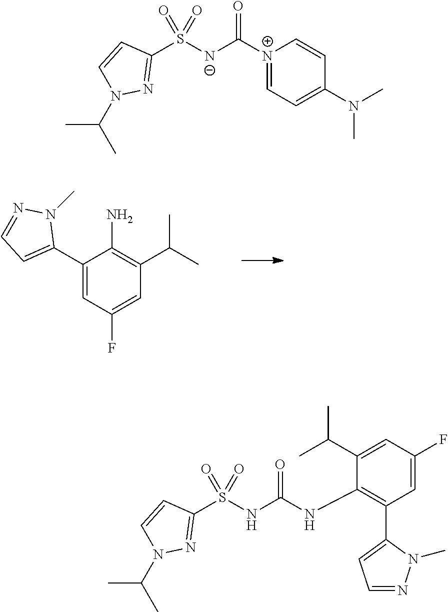

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(1-methyl-1H-pyrazol-5-yl)aniline (Intermediate A5) to afford the title compound (44 mg, 64%) as a white solid.

¹H NMR (DMSO-d₆) δ 10.90 (s, 1H), 7.96 (s, 1H), 7.73 (s, 1H), 7.38 (d, 1H), 7.25 (dd, 1H), 7.09 (d, 1H), 6.58 (s, 1H), 6.11 (d, 1H), 4.61 (sept, 1H), 3.55 (s, 3H), 3.08-2.86 (m, 1H), 1.45 (d, 6H) and 1.09 (d, 6H).

LCMS m/z 449.5 (M+H)⁺ (ES⁺); 447.4 (M−H)⁻ (ES⁻).

### Example 6: N-((4-Fluoro-2-iso-propyl-6-(thiazol-5-yl)phenyl)carbamoyl)-1-iso-propyl-1H-pyrazole-3-sulfonamide

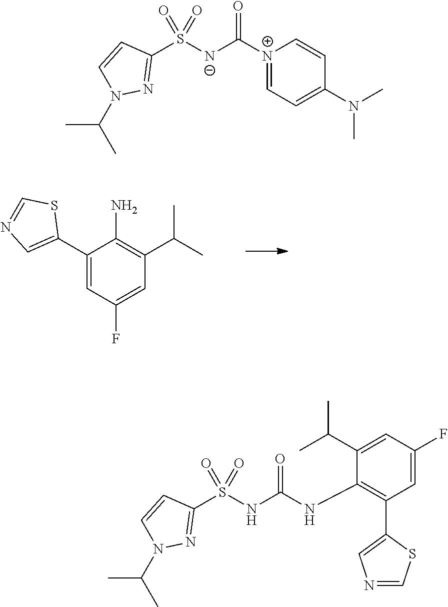

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(thiazol-5-yl)aniline (Intermediate A6) to afford the title compound (10 mg, 34%) as a white solid.

¹H NMR (DMSO-d₆) δ 11.20 (br s, 1H), 9.08 (s, 1H), 8.19 (s, 1H), 7.86 (s, 2H), 7.40 (dd, 1H), 7.21-7.08 (m, 1H), 6.56 (s, 1H), 4.77-4.29 (m, 1H), 3.10-2.88 (m, 1H), 1.42 (d, 6H) and 1.06 (s, 6H).

LCMS m/z 452.4 (M+H)⁺ (ES⁺); 450.2 (M−H)⁻ (ES⁻).

### Example 7: N-((4-Fluoro-2-iso-propyl-6-(isoxazol-4-yl)phenyl)carbamoyl)-1-iso-propyl-1H-pyrazole-3-sulfonamide

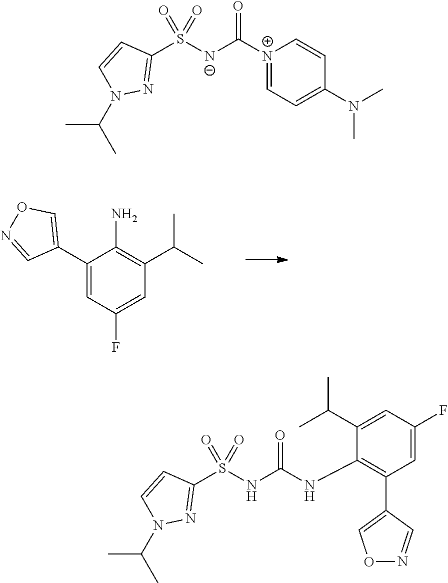

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(isoxazol-4-yl)aniline (Intermediate A7) to afford the title compound (23 mg, 57%) as a white solid.

¹H NMR (DMSO-d₆) δ 11.26 (s, 1H), 9.05 (s, 1H), 8.83 (s, 1H), 8.14 (s, 1H), 7.94 (d, 1H), 7.32 (dd, 1H), 7.15 (dd, 1H), 6.64 (d, 1H), 4.60 (sept, 1H), 3.06-2.95 (m, 1H), 1.43 (d, 6H) and 1.08 (br s, 6H).

LCMS 436.5 (M+H)⁺ (ES⁺); 434.3 (M−H)⁻ (ES⁻).

### Example 8: N-((3′-Cyano-5-fluoro-3-iso-propyl-[1,1′-biphenyl]-2-yl) carbamoyl)-1-iso-propyl-1H-pyrazole-3-sulfonamide, sodium salt

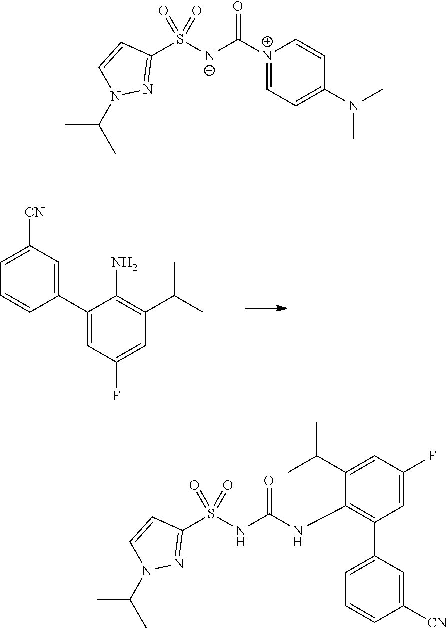

Prepared from (4-(dimethylamino)pyridin-1-ium-1-carbonyl)((1-isopropyl-1H-pyrazol-3-yl)sulfonyl)amide (Intermediate P3) and 2′-amino-5′-fluoro-3′-isopropyl-[1,1′-biphenyl]-3-carbonitrile (Intermediate A8) to afford the title compound (14.6 mg, 14%) as a colourless powder.

¹H NMR (DMSO-d₆) δ 7.78 (s, 1H), 7.75 (d, 1H), 7.66 (d, 1H), 7.64 (s, 1H), 7.45 (s, 1H), 7.42 (t, 1H), 7.09 (dd, 1H), 6.96 (dd, 1H), 6.16 (d, 1H), 4.48 (sept, 1H), 3.23-3.11 (m, 1H), 1.40 (d, 6H) and 1.08 (d, 6H).

LCMS m/z 470 (M+H)⁺ (ES⁺); 468 (M−H)⁻ (ES⁻).

### Example 9: N-((4′-Cyano-5-fluoro-3-iso-propyl-[1,1′-biphenyl]-2-yl) carbamoyl)-1-iso-propyl-1H-pyrazole-3-sulfonamide, sodium salt

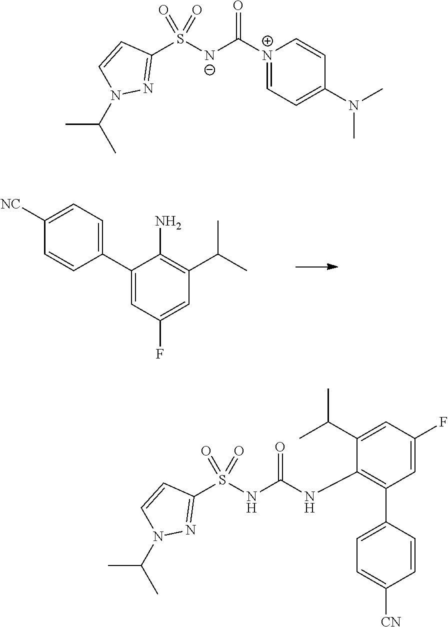

Prepared from (4-(dimethylamino)pyridin-1-ium-1-carbonyl)((1-isopropyl-1H-pyrazol-3-yl)sulfonyl)amide (Intermediate P3) and 2′-amino-5′-fluoro-3′-isopropyl-[1,1′-biphenyl]-4-carbonitrile (Intermediate A9) to afford the title compound (47.4 mg, 48%) as a colourless powder.

¹H NMR (DMSO-d₆) δ 7.72 (s, 1H), 7.67 (d, 2H), 7.52 (d, 2H), 7.39 (s, 1H), 7.11 (dd, 1H), 6.93 (dd, 1H), 6.24 (d, 1H), 4.51 (sept, 1H), 3.19 (br s, 1H), 1.42 (d, 6H) and 1.09 (d, 6H).

LCMS m/z 470 (M+H)⁺ (ES⁺); 468 (M−H)⁻ (ES⁻).

### Example 10: N-((4-Fluoro-2-iso-propyl-6-(pyridin-4-yl)phenyl)carbamoyl)-1-iso-propyl-1H-pyrazole-3-sulfonamide, partial ammonium salt

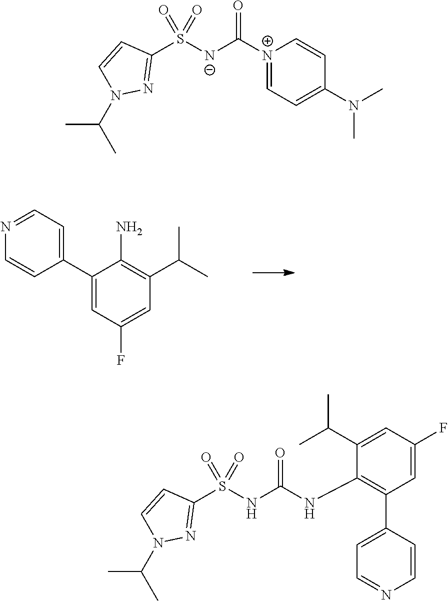

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(pyridin-4-yl)aniline (Intermediate A10) to afford the title compound (24 mg, 36%) as a white solid.

¹H NMR (DMSO-d₆) δ 8.55-8.34 (m, 2H), 7.89 (s, 1H), 7.79 (s, 1H), 7.31 (d, 2H), 7.21 (dd, 1H), 7.03 (dd, 1H), 6.49 (s, 1H), 4.57 (sept, 1H), 3.12-2.95 (m, 1H), 1.43 (d, 6H) and 1.09 (d, 6H). One exchangeable signal as a very broad singlet 11.25-10.00 ppm.

LCMS m/z 446.4 (M+H)⁺ (ES⁺); 444.1 (M−H)⁻ (ES⁻).

### Example 11: N-((2-(1,3-Dimethyl-1H-pyrazol-5-yl)-4-fluoro-6-iso-propylphenyl)carbamoyl)-1-iso-propyl-1H-pyrazole-3-sulfonamide, partial ammonium salt

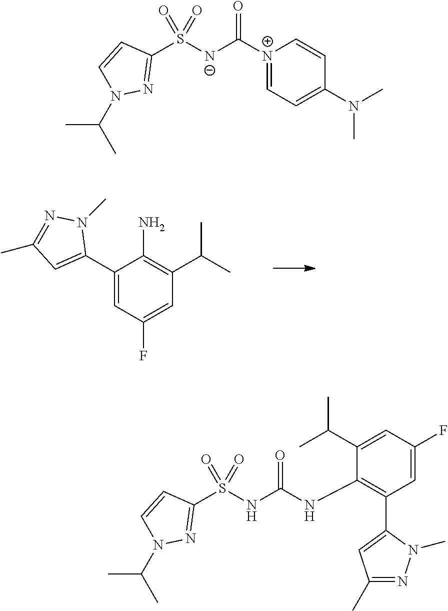

Prepared from (4-(dimethylamino)pyridin-1-ium-1-carbonyl)((1-isopropyl-1H-pyrazol-3-yl)sulfonyl)amide (Intermediate P3) and 2-(1,3-dimethyl-1H-pyrazol-5-yl)-4-fluoro-6-isopropylaniline (Intermediate A11) to afford the title compound (41 mg, 57%) as a white solid.

¹H NMR (DMSO-d₆) δ 7.84 (s, 1H), 7.53 (s, 1H), 7.19 (dd, 1H), 6.97 (dd, 1H), 6.45 (s, 1H), 5.94 (s, 1H), 4.55 (sept, 1H), 3.45 (s, 3H), 3.10-2.95 (m, 1H), 2.13 (s, 3H), 1.43 (d, 6H) and 1.08 (d, J=6.8 Hz, 6H); one exchangeable signal not observed.

LCMS m/z 463.4 (M+H)⁺ (ES⁺); 461.3 (M−H)⁻ (ES⁻).

### Example 12: N-((4-Fluoro-2-iso-propyl-6-(2-methoxypyridin-4-yl)phenyl) carbamoyl)-1-iso-propyl-1H-pyrazole-3-sulfonamide, partial ammonium salt

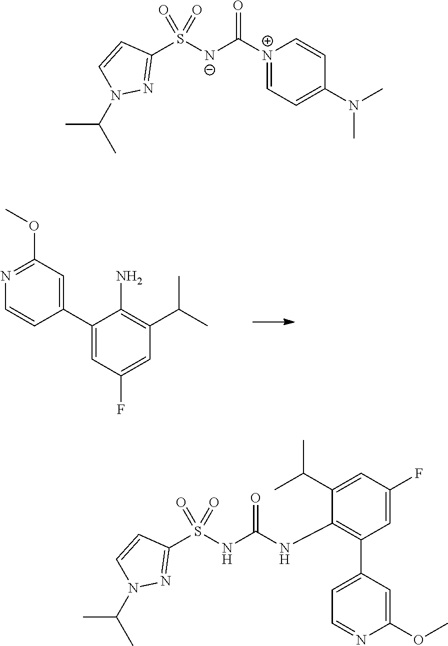

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(2-methoxy-pyridin-4-yl)aniline (Intermediate A12) to afford the title compound (32 mg, 44%) as a white solid.

¹H NMR (DMSO-d₆) δ 7.99 (d, 1H), 7.78 (s, 1H), 7.69 (s, 1H), 7.11 (dd, 1H), 6.93 (dd, 1H), 6.83 (d, 1H), 6.70 (s, 1H), 6.40 (s, 1H), 4.48 (sept, 1H), 3.80 (s, 3H), 3.02-2.82 (m, 1H), 1.35 (d, 6H) and 1.00 (d, 6H); one exchangeable signal not observed.

LCMS m/z 476.4 (M+H)⁺ (ES⁺); 474.3 (M−H)⁻ (ES⁻).

### Example 13: N-((4-Fluoro-2-iso-propyl-6-(2-methylpyridin-4-yl)phenyl) carbamoyl)-1-iso-propyl-1H-pyrazole-3-sulfonamide, partial ammonium salt

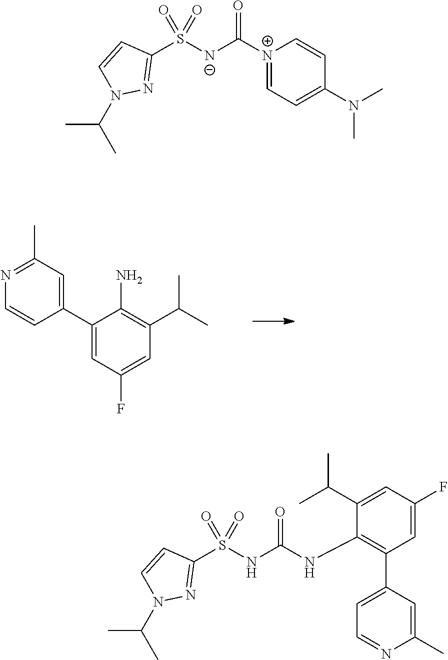

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(2-methylpyridin-4-yl)aniline (Intermediate A13) to afford the title compound (37 mg, 53%) as a white solid.

¹H NMR (DMSO-d₆) δ 8.33 (d, 1H), 7.83 (s, 1H), 7.69 (s, 1H), 7.21 (s, 1H), 7.17 (dd, 1H), 7.11 (d, 1H), 7.05-6.89 (m, 1H), 6.44 (s, 1H), 4.54 (sept, 1H), 3.15-2.96 (m, 1H), 2.45 (s, 3H), 1.42 (d, 6H) and 1.08 (d, 6H); one exchangeable signal not observed.

LCMS m/z 460.5 (M+H)⁺ (ES⁺); 458.4 (M−H)⁻ (ES⁻).

### Example 14: N-((4-Fluoro-2-iso-propyl-6-(2-methylpyridin-3-yl)phenyl) carbamoyl)-1-iso-propyl-1H-pyrazole-3-sulfonamide, partial ammonium salt

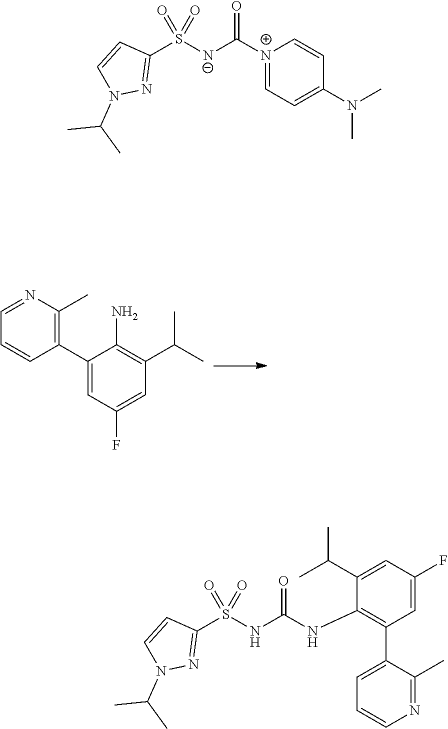

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(2-methylpyridin-3-yl)aniline (Intermediate A14) to afford the title compound (8 mg, 11%) as a white solid.

¹H NMR (DMSO-d₆) δ 8.39 (dd, 1H), 7.83 (s, 1H), 7.54 (s, 1H), 7.46-7.32 (m, 1H), 7.21-7.03 (m, 2H), 7.02-6.79 (m, 1H), 6.34 (s, 1H), 4.54 (sept, 1H), 3.16-2.93 (m, 1H), 2.19 (s, 3H), 1.43 (d, 6H) and 1.17-1.04 (m, 6H); one exchangeable signal not observed.

LCMS m/z 460.5 (M+H)⁺ (ES⁺); 458.4 (M−H)⁻ (ES⁻).

### Example 15: N-((4-Fluoro-2-iso-propyl-6-(6-methylpyridin-3-yl)phenyl) carbamoyl)-1-iso-propyl-1H-pyrazole-3-sulfonamide, partial ammonium salt

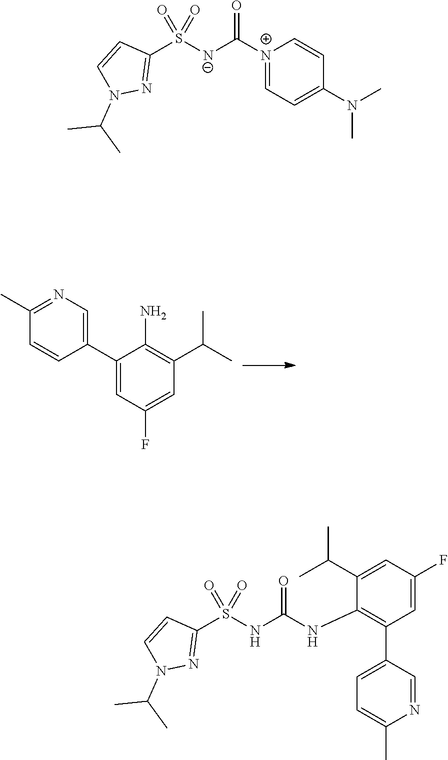

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(6-methylpyridin-3-yl)aniline (Intermediate A15) to afford the title compound (21 mg, 30%) as a white solid.

¹H NMR (DMSO-d₆) δ 8.38 (s, 1H), 7.84 (s, 1H), 7.66 (s, 1H), 7.61 (d, 1H), 7.19 (d, 1H), 7.13 (dd, 1H), 6.99 (dd, 1H), 6.44 (s, 1H), 4.56 (sept, 1H), 3.14-2.88 (m, 1H), 2.50 (s, 3H), 1.42 (d, 6H) and 1.07 (d, 6H); one exchangeable signal not observed.

LCMS m/z 460.5 (M+H)⁺ (ES⁺); 458.3 (M−H)⁻ (ES⁻).

### Example 16: N-((2-(5-Chloropyridin-3-yl)-4-fluoro-6-iso-propylphenyl) carbamoyl)-1-iso-propyl-1H-pyrazole-3-sulfonamide, partial ammonium salt

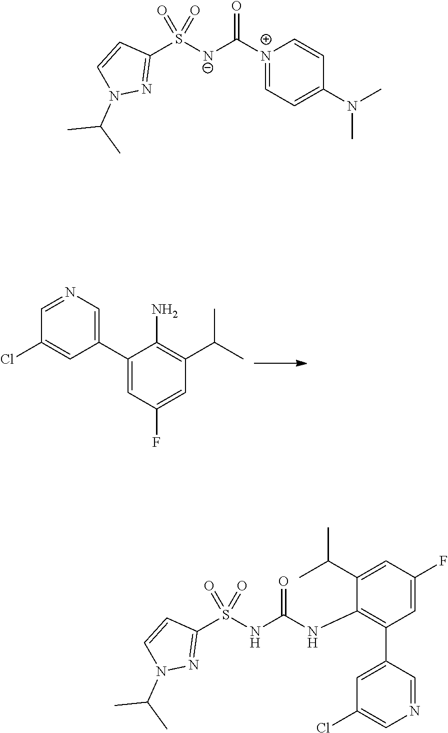

Prepared from (4-(dimethylamino)pyridin-1-ium-1-carbonyl)((1-isopropyl-1H-pyrazol-3-yl)sulfonyl)amide (Intermediate P3) and 2-(5-chloropyridin-3-yl)-4-fluoro-6-isopropylaniline (Intermediate A16) to afford the title compound (43.2 mg, 58%) as a white solid.

¹H NMR (DMSO-d₆) δ 8.57 (d, 1H), 8.46 (s, 1H), 7.98-7.79 (m, 3H), 7.21 (dd, 1H), 7.11 (dd, 1H), 6.45 (d, 1H), 4.56 (sept, 1H), 3.07-2.92 (m, 1H), 1.42 (d, 6H) and 1.09 (d, 6H).

LCMS m/z 480.4/482.4 (M+H)⁺ (ES⁺); 478.3/480.3 (M−H)⁻ (ES⁻).

### Example 17: N-((4-Fluoro-2-iso-propyl-6-(5-methoxypyridin-3-yl)phenyl) carbamoyl)-1-iso-propyl-1H-pyrazole-3-sulfonamide, partial ammonium salt

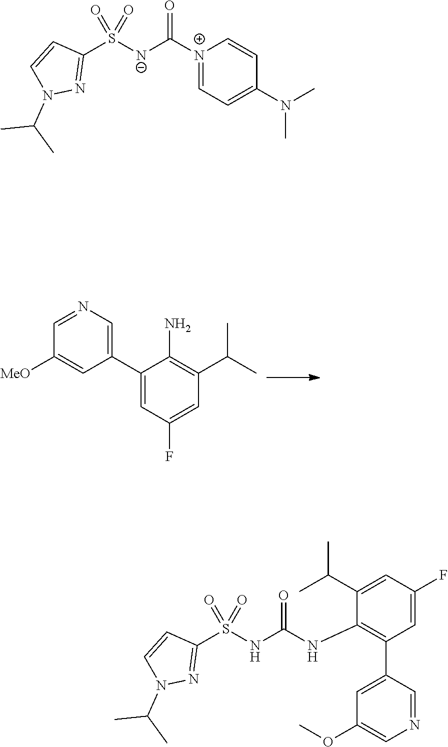

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(5-methoxy-pyridin-3-yl)aniline (Intermediate A17) to afford the title compound (44.6 mg, 60%) as a white solid.

¹H NMR (DMSO-d₆) δ 8.24 (d, 1H), 8.09 (d, 1H), 7.93-7.75 (m, 2H), 7.38 (s, 1H), 7.18 (dd, 1H), 7.06 (dd, 1H), 6.50 (s, 1H), 4.57 (sept, 1H), 3.82 (s, 3H), 3.06-2.89 (m, 1H), 1.43 (d, 6H) and 1.08 (d, 6H).

LCMS m/z 476.4 (M+H)⁺ (ES⁺); 474.5 (M−H)⁻ (ES⁻).

### Example 18: N-((4-Fluoro-2-iso-propyl-6-(pyrimidin-5-yl)phenyl) carbamoyl)-1-iso-propyl-1H-pyrazole-3-sulfonamide

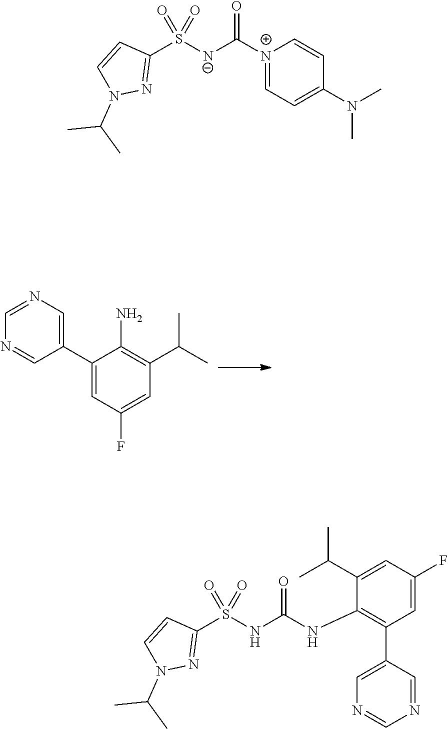

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(pyrimidin-5-yl)aniline (Intermediate A18) to afford the title compound (24.7 mg, 25%) as a colourless solid.

¹H NMR (DMSO-d₆) δ 11.06 (s, 1H), 9.13 (s, 1H), 8.75 (s, 2H), 8.01 (s, 1H), 7.90 (s, 1H), 7.25 (dd, 1H), 7.18 (dd, 1H), 6.49 (s, 1H), 4.59 (sept, 1H), 3.04 (sept, 1H), 1.44 (d, 6H) and 1.10 (d, 6H).

LCMS m/z 447 (M+H)⁺ (ES⁺); 445 (M−H)⁻ (ES⁻).

### Example 19: N-((4-Fluoro-2-iso-propyl-6-(6-methoxypyridin-3-yl)phenyl) carbamoyl)-1-iso-propyl-1H-pyrazole-3-sulfonamide

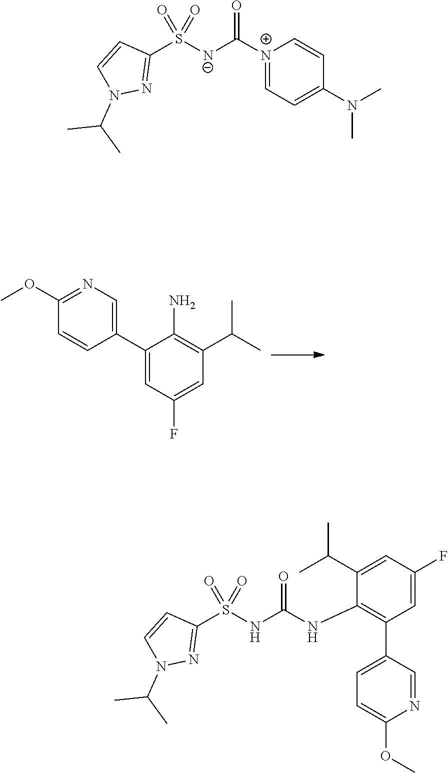

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(6-methoxy-pyridin-3-yl)aniline (Intermediate A19) to afford the title compound (29 mg, 53%) as a white solid.

¹H NMR (DMSO-d₆) δ 10.90 (s, 1H), 8.10 (d, 1H), 7.91 (s, 1H), 7.80 (s, 1H), 7.63 (dd, 1H), 7.15 (dd, 1H), 7.01 (dd, 1H), 6.74 (d, 1H), 6.55 (s, 1H), 4.59 (sept, 1H), 3.89 (s, 3H), 3.07-2.86 (m, 1H), 1.43 (d, 6H) and 1.08 (d, 6H).

LCMS m/z 476.5 (M+H)⁺ (ES⁺); 474.4 (M−H)⁻ (ES⁻).

### Example 20: N-((4-Fluoro-2-iso-propyl-6-(4-methylpyridin-3-yl)phenyl) carbamoyl)-1-iso-propyl-1H-pyrazole-3-sulfonamide, partial ammonium salt

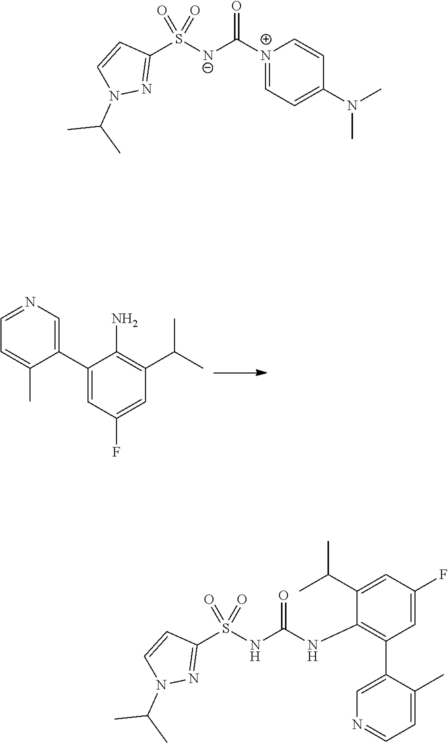

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(4-methylpyridin-3-yl)aniline (Intermediate Ago) to afford the title compound (23 mg, 40%) as a white solid.

¹H NMR (DMSO-d₆) δ 8.40-8.34 (m, 1H), 8.32 (s, 1H), 7.85 (s, 1H), 7.74 (s, 1H), 7.57 (s, 1H), 7.16 (dd, 1H), 7.02 (dd, 1H), 6.45 (s, 1H), 4.55 (sept, 1H), 3.12-2.93 (m, 1H), 2.29 (s, 3H), 1.42 (d, 6H) and 1.08 (d, 6H); one exchangeable signal not observed.

LCMS m/z 460.6 (M+H)⁺ (ES⁺); 458.4 (M−H)⁻ (ES⁻).

### Example 21: N-((4-Fluoro-2-(5-fluoropyridin-3-yl)-6-isopropylphenyl) carbamoyl)-1-iso-propyl-1H-pyrazole-3-sulfonamide, partial ammonium salt

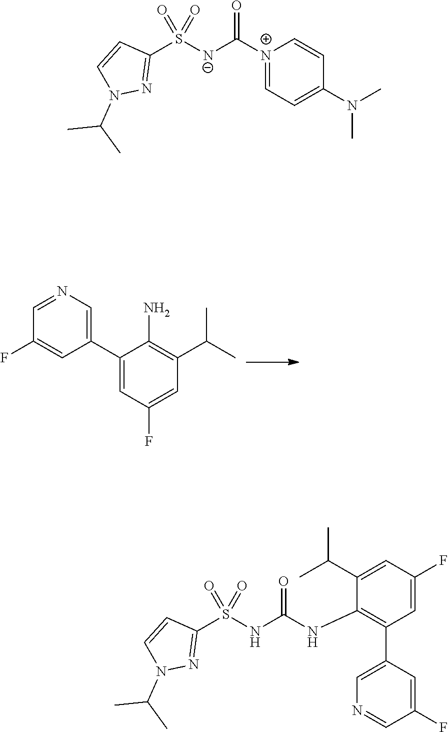

Prepared from (4-(dimethylamino)pyridin-1-ium-1-carbonyl)((1-isopropyl-1H-pyrazol-3-yl)sulfonyl)amide (Intermediate P3) and 4-fluoro-2-(5-fluoropyridin-3-yl)-6-isopropylaniline (Intermediate A21) to afford the title compound (14.1 mg, 21%) as a colourless solid.

¹H NMR (DMSO-d₆) δ 8.51 (d, 1H), 8.41 (s, 1H), 7.81-7.63 (m, 3H), 7.17 (dd, 1H), 7.07 (dd, 1H), 6.31 (s, 1H), 4.51 (sept, 1H), 3.21-3.04 (m, 1H), 1.41 (d, 6H) and 1.09 (d, 6H); one exchangeable signal not observed.

LCMS m/z 464 (M+H)⁺ (ES⁺); 462 (M−H)⁻ (ES⁻).

### Example 22: N-((4-Fluoro-2-iso-propyl-6-(3-methylpyridin-4-yl)phenyl) carbamoyl)-1-iso-propyl-1H-pyrazole-3-sulfonamide

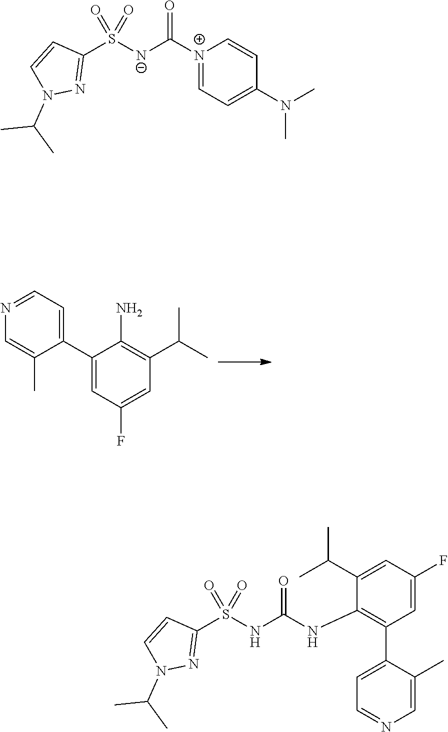

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(3-methylpyridin-4-yl)aniline (Intermediate A22) to afford the title compound (27.9 mg, 41%) as a colourless powder.

¹H NMR (DMSO-d₆) δ 10.75 (s, 1H), 8.42 (s, 1H), 8.28 (d, 1H), 7.96 (s, 1H), 7.70 (s, 1H), 7.22 (dd, 1H), 7.02 (s, 1H), 6.93 (dd, 1H), 6.49 (s, 1H), 4.60 (sept, 1H), 2.98 (sept, 1H), 2.00 (s, 3H), 1.45 (d, 6H) and 1.11 (d, 6H).

LCMS m/z 460 (M+H)⁺ (ES⁺); 458 (M−H)⁻ (ES⁻).

### Example 23: N-((2-(2-Aminopyridin-4-yl)-4-fluoro-6-isopropylphenyl) carbamoyl)-1-iso-propyl-1H-pyrazole-3-sulfonamide

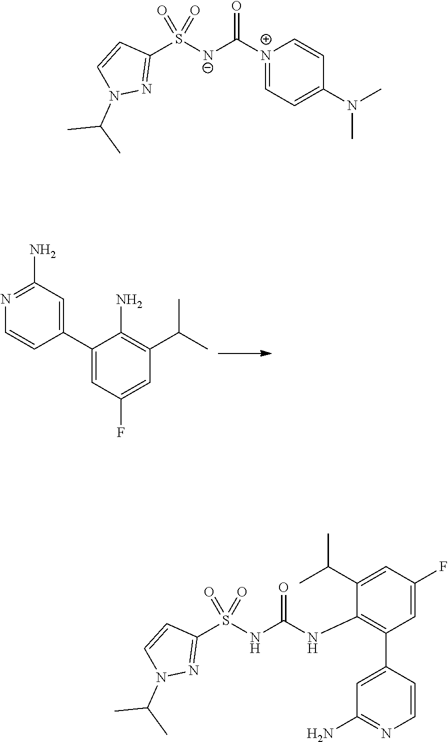

Prepared from (4-(dimethylamino)pyridin-1-ium-1-carbonyl)((1-isopropyl-1H-pyrazol-3-yl)sulfonyl)amide (Intermediate P3) and 4-(2-amino-5-fluoro-3-isopropylphenyl) pyridin-2-amine (Intermediate A23) to afford the title compound (19 mg, 27%) as a white solid.

¹H NMR (DMSO-d₆) δ 10.90 (br s, 1H), 7.95 (d, 1H), 7.78 (dd, 1H), 7.70 (s, 1H), 7.18 (dd, 1H), 6.93 (dd, 1H), 6.58 (d, 1H), 6.41-6.35 (m, 1H), 6.32 (s, 1H), 5.95 (br s, 2H), 4.60 (sept, 1H), 3.06-2.83 (m, 1H), 1.44 (d, 6H) and 1.07 (d, 6H).

LCMS m/z 461.5 (M+H)⁺ (ES⁺); 459.3 (M−H)⁻ (ES⁻).

### Example 24: N-((2-(2-Ethoxypyridin-4-yl)-4-fluoro-6-iso-propylphenyl) carbamoyl)-1-iso-propyl-1H-pyrazole-3-sulfonamide

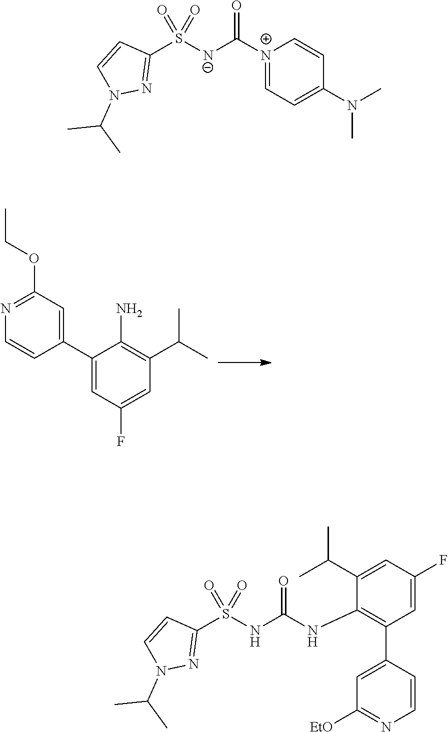

Prepared from (4-(dimethylamino)pyridin-1-ium-1-carbonyl)((1-isopropyl-1H-pyrazol-3-yl)sulfonyl)amide (Intermediate P3) and 2-(2-ethoxypyridin-4-yl)-4-fluoro-6-isopropylaniline (Intermediate A24) to afford the title compound (20 mg, 27%) as a white solid.

¹H NMR (DMSO-d₆) δ 10.94 (s, 1H), 8.06 (d, 1H), 7.92 (s, 1H), 7.85 (s, 1H), 7.20 (dd, 1H), 7.02 (dd, 1H), 6.93-6.79 (m, 1H), 6.73 (d, 1H), 6.55 (s, 1H), 4.59 (sept, 1H), 4.32 (q, 2H), 3.07-2.88 (m, 1H), 1.43 (d, 6H), 1.34 (t, 3H) and 1.20-0.88 (m, 6H).

LCMS m/z 490.5 (M+H)⁺ (ES⁺); 488.3 (M−H)⁻ (ES⁻).

### Example 25: N-((4-Fluoro-2-(2-hydroxypyridin-4-yl)-6-iso-propylphenyl) carbamoyl)-1-iso-propyl-1H-pyrazole-3-sulfonamide

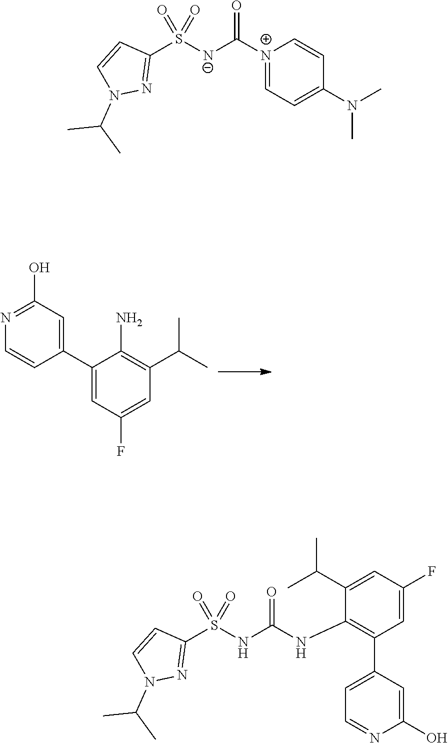

Prepared from (4-(dimethylamino)pyridin-1-ium-1-carbonyl)((1-isopropyl-1H-pyrazol-3-yl)sulfonyl)amide (Intermediate P3) and 4-(2-amino-5-fluoro-3-isopropylphenyl) pyridin-2-ol (Intermediate A25) to afford the title compound (10.5 mg, 15%) as a colourless powder.

¹H NMR (DMSO-d₆) δ 11.89 (s, 1H), 7.75 (br s, 2H), 7.23 (d, 1H), 7.13 (dd, 1H), 6.92 (dd, 1H), 6.45 (s, 1H), 6.18 (s, 1H), 6.07 (d, 1H), 4.54 (sept, 1H), 3.21-3.02 (m, 1H), 1.40 (d, 6H) and 1.08 (d, 6H); one exchangeable signal not observed.

LCMS m/z 462 (M+H)⁺ (ES⁺); 460 (M−H)⁻ (ES⁻).

### Example 26: N-((4-Fluoro-2-iso-propyl-6-(2-methoxy-6-methylpyridin-4-yl)phenyl)carbamoyl)-1-iso-propyl-1H-pyrazole-3-sulfonamide

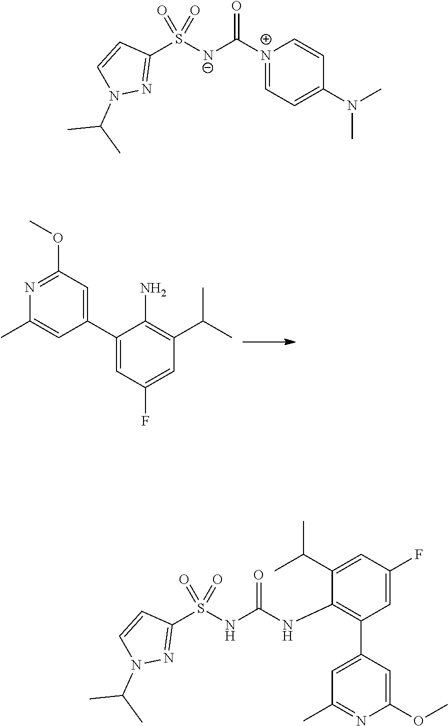

Prepared from (4-(dimethylamino)pyridin-1-ium-1-carbonyl)((1-isopropyl-1H-pyrazol-3-yl)sulfonyl)amide (Intermediate P3) and 4-fluoro-2-isopropyl-6-(2-methoxy-6-methylpyridin-4-yl)aniline (Intermediate A26) to afford the title compound (16.7 mg, 23%) as a colourless powder.

¹H NMR (DMSO-d₆) δ 7.74 (s, 1H), 7.57 (s, 1H), 7.13 (dd, 1H), 6.94 (dd, 1H), 6.82 (s, 1H), 6.60 (s, 1H), 6.35 (s, 1H), 4.51 (sept, 1H), 3.85 (s, 3H), 3.19-3.02 (m, 1H), 2.36 (s, 3H), 1.41 (d, 6H) and 1.08 (d, 6H).

LCMS m/z 490 (M+H)⁺ (ES⁺); 488 (M−H)⁻ (ES⁻).

### Example 27: N-((4-Fluoro-2-(2-isopropoxypyridin-4-yl)-6-iso-propyl-phenyl)carbamoyl)-1-iso-propyl-1H-pyrazole-3-sulfonamide

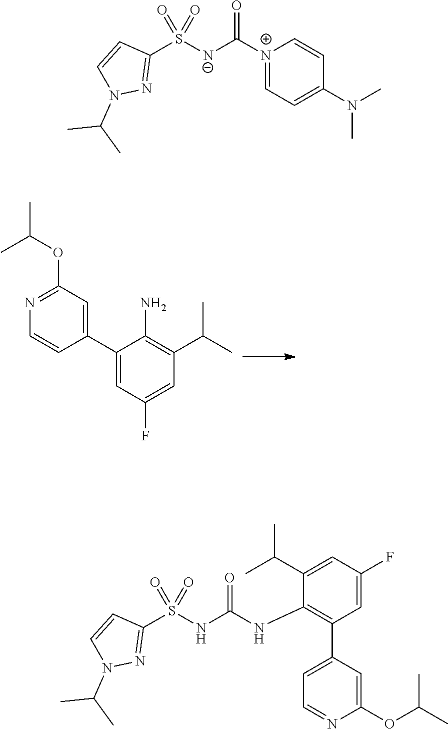

Prepared from (4-(dimethylamino)pyridin-1-ium-1-carbonyl)((1-isopropyl-1H-pyrazol-3-yl)sulfonyl)amide (Intermediate P3) and 4-fluoro-2-(2-isopropoxy-pyridin-4-yl)-6-isopropylaniline (Intermediate A27) to afford the title compound (31.6 mg, 42%) as a colourless powder.

¹H NMR (DMSO-d₆) δ 8.04 (d, 1H), 7.87 (s, 1H), 7.75 (s, 1H), 7.18 (dd, 1H), 7.01 (dd, 1H), 6.86 (d, 1H), 6.70 (s, 1H), 6.50 (s, 1H), 5.27 (sept, 1H), 4.57 (sept, 1H), 3.14-2.89 (m, 1H), 1.43 (d, 6H), 1.32 (d, 6H) and 1.08 (d, 6H).

LCMS m/z 504 (M+H)⁺ (ES⁺); 502 (M−H)⁻ (ES⁻).

### Example 28: N-((2-(2-Cyanopyridin-4-yl)-4-fluoro-6-iso-propylphenyl) carbamoyl)-1-iso-propyl-1H-pyrazole-3-sulfonamide

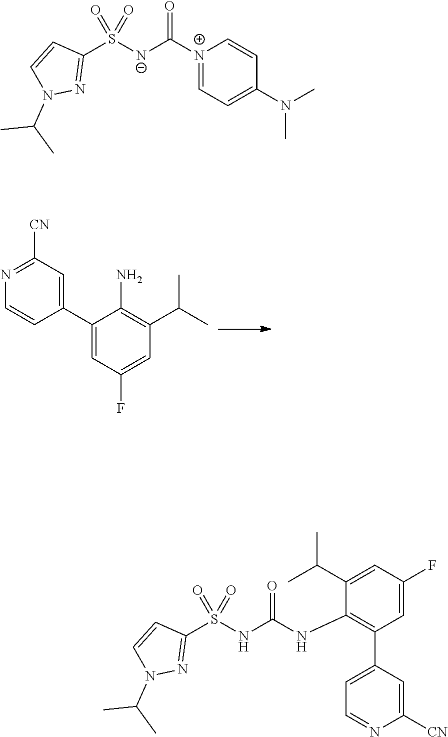

Prepared from (4-(dimethylamino)pyridin-1-ium-1-carbonyl)((1-isopropyl-1H-pyrazol-3-yl)sulfonyl)amide (Intermediate P3) and 4-(2-amino-5-fluoro-3-isopropylphenyl)-picolinonitrile (Intermediate A28) to afford the title compound (18.5 mg, 26%) as a colourless powder.

¹H NMR (DMSO-d₆) δ 11.07 (s, 1H), 8.67 (d, 1H), 8.06-8.01 (m, 2H), 7.85 (d, 1H), 7.67 (dd, 1H), 7.27 (dd, 1H), 7.15 (dd, 1H), 6.43 (s, 1H), 4.56 (sept, 1H), 3.18-2.96 (m, 1H), 1.43 (d, 6H) and 1.11 (d, 6H).

LCMS m/z 471 (M+H)⁺ (ES+); 469 (M−H)− (ES−).

### Example 29: N-((2-(2-Ethylpyridin-4-yl)-4-fluoro-6-iso-propylphenyl) carbamoyl)-1-iso-propyl-1H-pyrazole-3-sulfonamide

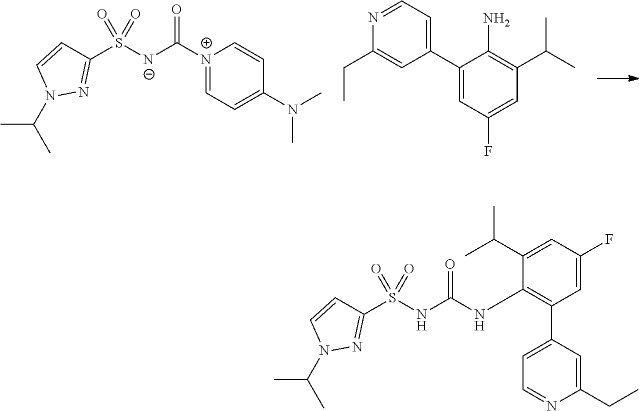

Prepared from (4-(dimethylamino)pyridin-1-ium-1-carbonyl)((1-isopropyl-1H-pyrazol-3-yl)sulfonyl)amide (Intermediate P3) and 2-(2-ethylpyridin-4-yl)-4-fluoro-6-isopropylaniline (Intermediate A29) to afford the title compound (27.8 mg, 39%) as a colourless powder.

¹H NMR (DMSO-d₆) δ 10.92 (s, 1H), 8.41 (dd, 1H), 7.95 (d, 1H), 7.88 (s, 1H), 7.26-7.20 (m, 2H), 7.11 (dd, 1H), 7.06 (dd, 1H), 6.58 (d, 1H), 4.60 (sept, 1H), 2.97 (sept, 1H), 2.75 (q, 2H), 1.44 (d, 6H), 1.25 (t, 3H) and 1.09 (br s, 6H).

LCMS m/z 474 (M+H)⁺ (ES⁺); 472 (M−H)⁻ (ES⁻).

### Example 30: 3-(N-((4-Fluoro-2-iso-propyl-6-(tetrahydro-2H-pyran-4-yl)phenyl)carbamoyl)sulfamoyl)-N,N,1-trimethyl-1H-pyrazole-5-carboxamide, sodium salt

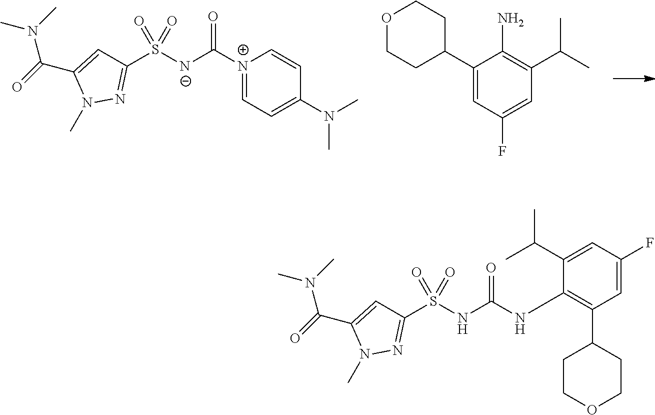

Prepared from (4-(dimethylamino)pyridin-1-ium-1-carbonyl)((5-(dimethylcarbamoyl)-1-methyl-1H-pyrazol-3-yl)sulfonyl)amide (Intermediate P4) and 4-fluoro-2-isopropyl-6-(tetrahydro-2H-pyran-4-yl)aniline (Intermediate A30) to afford the title compound (5 mg, 5%) as a solid.

¹H NMR (DMSO-d₆) δ 7.39 (s, 1H), 6.81 (td, 2H), 6.61 (s, 1H), 3.90-3.81 (m, 5H), 3.28-3.11 (m, 3H), 3.04-2.97 (m, 7H), 1.57-1.43 (m, 4H) and 1.04 (d, 6H).

LCMS m/z 496.5 (M+H)⁺ (ES⁺); 494.3 (M−H)⁻ (ES⁻).

### Example 31: N-((4-Fluoro-2-iso-propyl-6-(1-methyl-1H-pyrazol-4-yl) phenyl)carbamoyl)-1-iso-propyl-1H-imidazole-4-sulfonamide

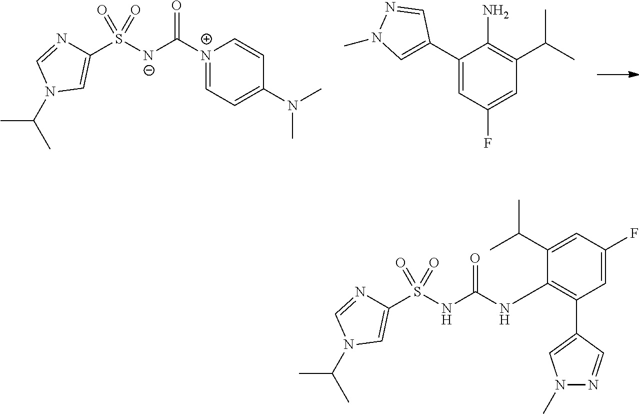 [SMILES: CC(C)n1cnc(S(=O)(=O)[N-]C(=O)[n+]2ccc(N(C)C)cc2)c1 ; CC(C)c1cc(F)cc(-c2cnn(C)c2)c1N ; CC(C)c1cc(F)cc(-c2cnn(C)c2)c1NC(=O)NS(=O)(=O)c1cn(C(C)C)cn1]

Prepared from (4-(dimethylamino)pyridin-1-ium-1-carbonyl)((1-isopropyl-1H-imidazol-4-yl)sulfonyl)amide (Intermediate P6) and 4-fluoro-2-isopropyl-6-(1-methyl-1H-pyrazol-4-yl)aniline (Intermediate A2) to afford the title compound (24.9 mg, 37%) as a white solid.

¹H NMR (DMSO-d₆) δ 7.95 (s, 1H), 7.90 (s, 1H), 7.81 (s, 1H), 7.68 (s, 1H), 7.68-7.64 (m, 1H), 7.14 (dd, 1H), 6.94 (dd, 1H), 4.44 (sept, 1H), 3.87 (s, 3H), 3.14-2.87 (m, 1H), 1.38 (d, 6H) and 1.04 (d, 6H); one exchangeable signal not observed.

LCMS m/z 449.4 (M+H)⁺ (ES⁺); 447.2 (M−H)⁻ (ES⁻).

### Example 32: 3-(N-((4-Fluoro-2-iso-propyl-6-(pyrimidin-5-yl)phenyl) carbamoyl)sulfamoyl)-N,N,1-trimethyl-1H-pyrazole-5-carboxamide, sodium salt

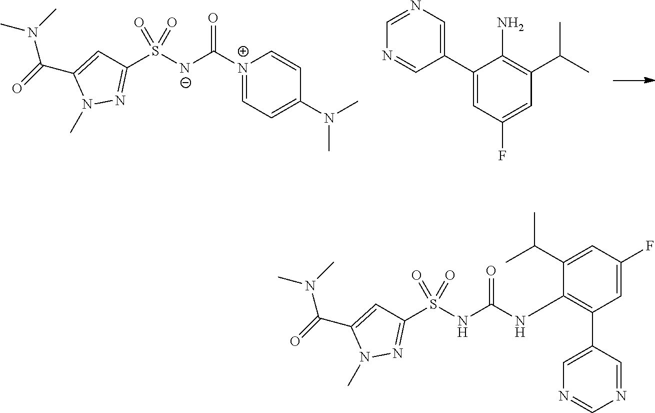

Prepared from (4-(dimethylamino)pyridin-1-ium-1-carbonyl)((5-(dimethylcarbamoyl)-1-methyl-1H-pyrazol-3-yl)sulfonyl)amide (Intermediate P4) and 4-fluoro-2-isopropyl-6-(pyrimidin-5-yl)aniline (Intermediate A18) to afford the title compound (31 mg, ii %) as a white solid.

¹H NMR (DMSO-d₆) δ 9.03 (s, 1H), 8.76 (s, 2H), 7.30 (br s, 1H), 7.11 (dd, 1H), 7.03 (dd, 1H), 6.43 (s, 1H), 3.85 (s, 3H), 3.26 (sept, 1H), 3.04 (s, 6H) and 1.14 (d, 6H).

LCMS m/z 490.4 (M+H)⁺ (ES⁺).

### Example 33: 3-(N-((4-Fluoro-2-iso-propyl-6-(pyridin-3-yl)phenyl) carbamoyl)sulfamoyl)-N,N,1-trimethyl-1H-pyrazole-5-carboxamide, sodium salt

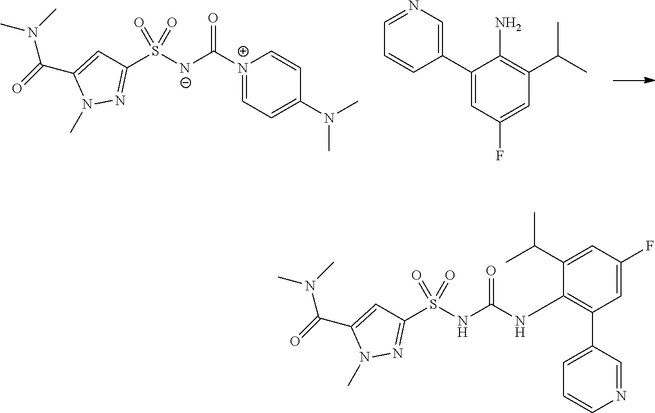

Prepared from (4-(dimethylamino)pyridin-1-ium-1-carbonyl)((5-(dimethylcarbamoyl)-1-methyl-1H-pyrazol-3-yl)sulfonyl)amide (Intermediate P4) and 4-fluoro-2-isopropyl-6-(pyridin-3-yl)aniline (Intermediate A1) to afford the title compound (23 mg, 9%) as a white solid.

¹H NMR (DMSO-d₆) δ 8.55 (m, 1H), 8.45 (dd, 1H), 7.77 (dt, 1H), 7.25 (ddd, 1H), 7.06 (dd, 1H), 6.91 (dd, 1H), 6.44 (s, 1H), 3.84 (s, 3H), 3.26 (sept, 1H), 3.04 (s, 6H) and 1.13 (d, 6H).

LCMS m/z 489.4 (M+H)⁺ (ES+).

### Example 34: 3-(N-((4-Fluoro-2-iso-propyl-6-(1-methyl-1H-pyrazol-4-yl) phenyl)carbamoyl)sulfamoyl)-N,N,1-trimethyl-1H-pyrazole-5-carboxamide, sodium salt

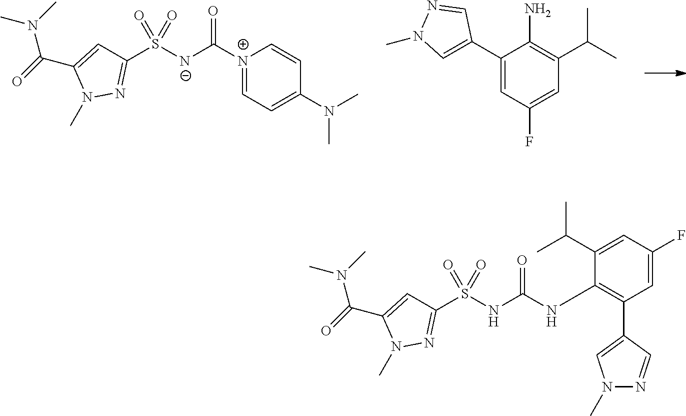

Prepared from (4-(dimethylamino)pyridin-1-ium-1-carbonyl)((5-(dimethylcarbamoyl)-1-methyl-1H-pyrazol-3-yl)sulfonyl)amide (Intermediate P4) and 4-fluoro-2-isopropyl-6-(1-methyl-1H-pyrazol-4-yl)aniline (Intermediate A2) to afford the title compound (40 mg, 21%) as a white solid.

¹H NMR (DMSO-d⁶) δ 7.95 (s, 1H), 7.76 (s, 1H), 7.25 (s, 1H), 7.10 (dd, 1H), 6.86 (dd, 1H), 6.58 (s, 1H), 3.82 (s, 3H), 3.80 (s, 3H), 3.20 (m, 1H), 2.99 (s, 6H) and 1.06 (d, 6H).

LCMS m/z 492.4 (M+H)⁺ (ES⁺); 490.3 (M−H)⁻ (ES⁻).

### Example 35: N-((4-Fluoro-2-iso-propyl-6-(pyridin-3-yl)phenyl)carbamoyl)-5-(2-methoxypropan-2-yl)-1-methyl-1H-pyrazole-3-sulfonamide

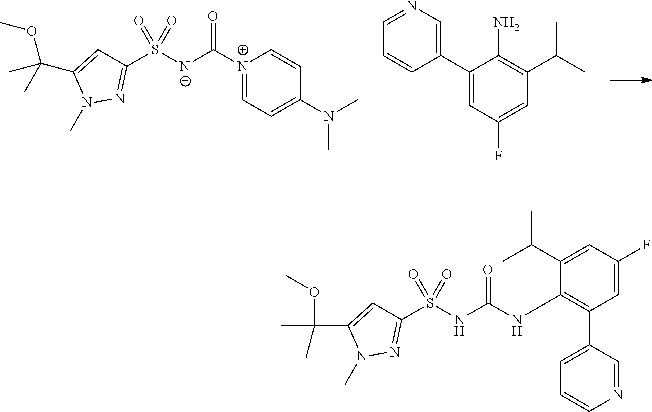

Prepared from (4-(dimethylamino)pyridin-1-ium-1-carbonyl)((5-(2-methoxypropan-2-yl)-1-methyl-1H-pyrazol-3-yl)sulfonyl)amide (Intermediate P5) and 4-fluoro-2-isopropyl-6-(pyridin-3-yl)aniline (Intermediate A1) to afford the title compound (7 mg, 13%) as a white solid.

¹H NMR (DMSO-d₆) δ 10.93 (br s, 1H), 8.55 (dd, 1H), 8.49 (d, 1H), 7.89 (s, 1H), 7.73 (dt, 1H), 7.38 (ddd. 1H), 7.22 (dd, 1H), 7.07 (dd, 1H), 6.56 (s, 1H), 4.00 (s, 3H), 3.11-2.99 (m, 1H), 2.99 (s, 3H), 1.51 (s, 6H) and 1.19-1.00 (br s, 6H).

LCMS m/z 490.4 (M+H)⁺ (ES⁺).

### Example 36: 5-((Dimethylamino)methyl)-N-((4-fluoro-2-iso-propyl-6-(2-methoxypyridin-4-yl)phenyl)carbamoyl)-1-methyl-1H-pyrazole-3-sulfonamide

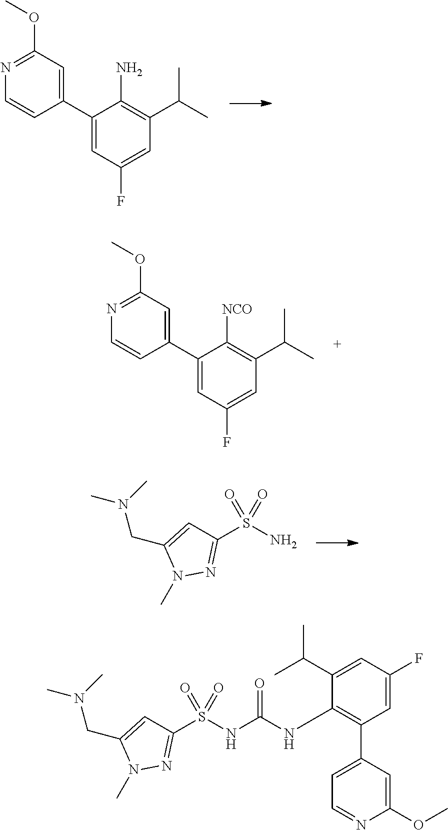

4-Fluoro-2-isopropyl-6-(2-methoxypyridin-4-yl)aniline (Intermediate A12; 0.1 g, 0.384 mmol) was dissolved in dry tetrahydrofuran (2 mL). Triethylamine (0.06 ml, 0.430 mmol) and a solution of triphosgene (0.108 g, 0.365 mmol) in tetrahydrofuran (1 mL) was added. The thick, opaque mixture was stirred overnight and then filtered through a phase cartridge washing with toluene (30 mL). After concentration in vacuo, 4-(5-fluoro-2-isocyanato-3-isopropylphenyl)-2-methoxypyridine was isolated as an oil. 5-((Dimethylamino)methyl)-1-methyl-1H-pyrazole-3-sulfonamide (Intermediate P1; 0.042 g, 0.192 mmol) was dissolved in dry tetrahydrofuran (1 mL). Sodium tert-butoxide (2 M in tetrahydrofuran; 0.1 ml, 0.200 mmol) was added and the mixture was stirred at room temperature for 1 hour. A solution of the previously prepared isocyanate (0.192 mmol) in tetrahydrofuran (1 mL) was added via syringe and the mixture was stirred overnight. The volatiles were removed in vacua and the residue was dissolved in dimethylsulfoxide (1 mL) and then purified by preparative HPLC (basic 6.5 minutes run, 10-40% acetonitrile in 10 mM aqueous ammonium bicarbonate) to afford the title compound (15.2 mg, 16%) as a colourless powder.

¹H NMR (DMSO-d₆) δ 10.87 (s, 1H), 8.09 (dd, 1H), 7.85 (s, 1H), 7.22 (dd, 1H), 7.04 (dd, 1H), 6.89 (dd, 1H), 6.77 (s, 1H), 6.51 (s, 1H), 3.88 (s, 6H), 3.49 (s, 2H), 3.02 (sept, 1H), 2.17 (s, 6H) and 1.09 (d, 6H).

LCMS m/z 505 (M+H)⁺ (ES⁺); 503 (M−H)⁻ (ES⁻).

### Example 37: 5-((Dimethylamino)methyl)-N-((4-fluoro-2-iso-propyl-6-(2-methoxypyridin-4-yl)phenyl)carbamoyl)-1-iso-propyl-1H-pyrazole-3-sulfonamide

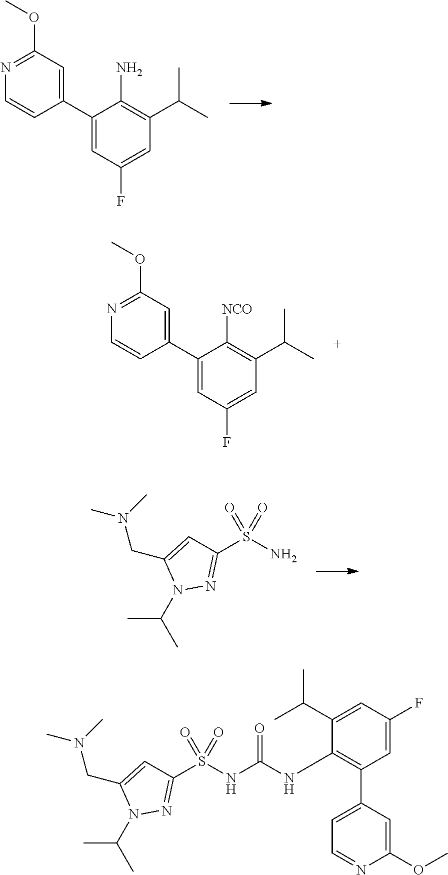

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-isopropyl-1H-pyrazole-3-sulfonamide (Intermediate P2) and 4-fluoro-2-isopropyl-6-(2-methoxypyridin-4-yl)aniline (Intermediate A12) to afford the title compound (44.1 mg, 43%) as a colourless powder.

¹H NMR (DMSO-d₆) δ 10.92 (s, 1H), 8.09 (dd, 1H), 7.87 (s, 1H), 7.22 (dd, 1H), 7.04 (dd, 1H), 6.92 (dd, 1H), 6.79 (s, 1H), 6.48 (s, 1H), 4.81 (sept, 1H), 3.88 (s, 3H), 3.48 (s, 2H), 2.98 (sept, 1H), 2.15 (s, 6H), 1.37 (d, 6H) and 1.08 (d, 6H).

LCMS m/z 533 (M+H)⁺ (ES⁺); 531 (M−H) (ES⁻).

### Example 38: N-((3′-Cyano-5-fluoro-3-iso-propyl-[1,1′-biphenyl]-2-yl)carbamoyl)-5-((dimethylamino)methyl)-1-methyl-1H-pyrazole-3-sulfonamide

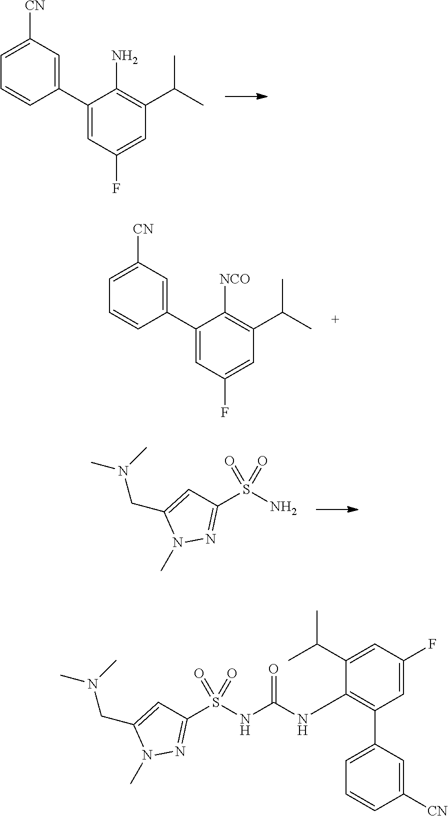

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-methyl-1H-pyrazole-3-sulfonamide (Intermediates P1) and 2′-amino-5′-fluoro-3′-isopropyl-[1,1′-biphenyl]-3-carbonitrile (Intermediate A8) to afford the title compound (37.3 mg, 34%) as a colourless powder.

¹H NMR (DMSO-d₆) δ 10.86 (s, 1H), 7.90 (s, 1H), 7.84-7.78 (m, 2H), 7.65-7.60 (m, 1H), 7.53 (t, 1H), 7.21 (dd, 1H), 7.06 (dd, 1H), 6.45 (s, 1H), 3.87 (s, 3H), 3.49 (s, 2H), 3.04 (sept, 1H), 2.17 (s, 6H) and 1.10 (br s, 6H).

LCMS m/z 499 (M+H)⁺ (ES⁺); 497 (M−H)⁻ (ES⁻).

### Example 39: N-((3′-Cyano-5-fluoro-3-iso-propyl-[1,1′-biphenyl]-2-yl)carbamoyl)-5-((dimethylamino)methyl)-1-isopropyl-1H-pyrazole-3-sulfonamide

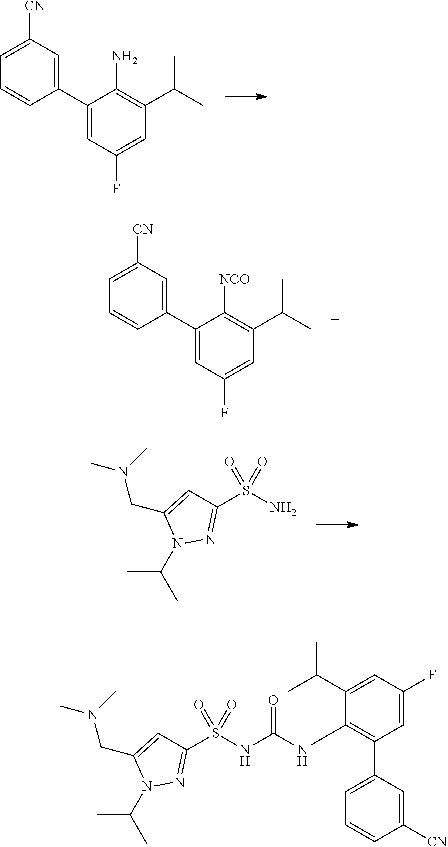

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-isopropyl-1H-pyrazole-3-sulfonamide (Intermediate P2) and 2′-amino-5′-fluoro-3′-isopropyl-[1,1′-biphenyl]-3-carbonitrile (Intermediate A8) to afford the title compound (27.6 mg, 24%) as a colourless powder.

¹H NMR (DMSO-d₆) δ 10.92 (s, 1H), 7.93 (s, 1H), 7.82 (dt, 2H), 7.66 (dt, 1H), 7.57-7.51 (m, 1H), 7.21 (dd, 1H), 7.07 (dd, 1H), 6.42 (s, 1H), 4.79 (sept, 1H), 3.48 (s, 2H), 3.00 (sept, 1H), 2.15 (s, 6H), 1.37 (d, 6H) and 1.09 (s, 6H).

LCMS m/z 527 (M+H)⁺ (ES⁺); 525 (M−H) (ES⁻).

### Example 40: 5-((Dimethylamino)methyl)-N-((5-fluoro-3-iso-propyl-[1,1′-biphenyl]-2-yl)carbamoyl)-1-methyl-1H-pyrazole-3-sulfonamide

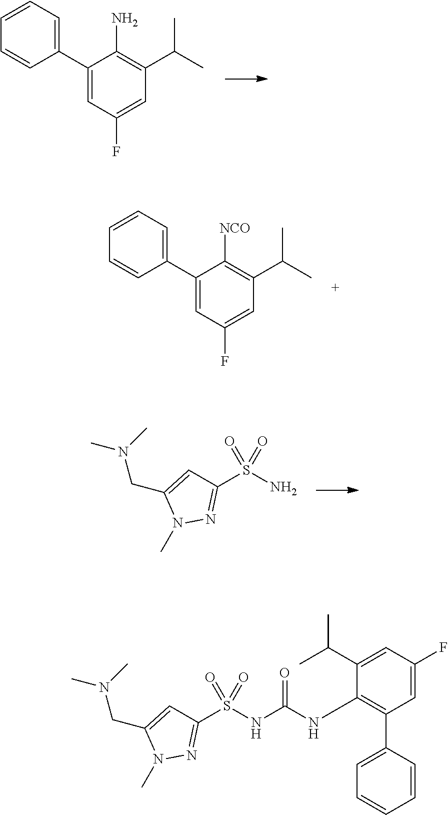

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-methyl-1H-pyrazole-3-sulfonamide (Intermediate P1) and 5-fluoro-3-isopropyl-[1,1′-biphenyl]-2-amine (Intermediate A4) to afford the title compound (14.2 mg, 14%) as a colourless solid.

¹H NMR (DMSO-d₆) δ 10.73 (s, 1H), 7.68 (s, 1H), 7.42-7.30 (m, 3H), 7.31-7.24 (m, 2H), 7.16 (dd, 1H), 6.96 (dd, 1H), 6.54 (s, 1H), 3.90 (s, 3H), 3.50 (s, 2H), 2.99 (sept, 1H), 2.17 (s, 6H) and 1.09 (d, 6H).

LCMS m/z 474 (M+H)⁺ (ES⁺); 472 (M−H)⁻ (ES⁻).

### Example 41: 5-((Dimethylamino)methyl)-N-((4-fluoro-2-iso-propyl-6-(pyridin-3-yl)phenyl)carbamoyl)-1-iso-propyl-1H-pyrazole-3-sulfonamide

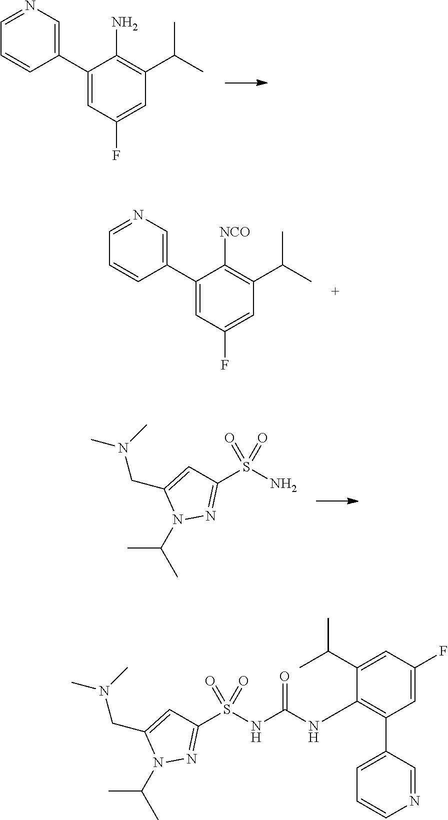

5-((Dimethylamino)methyl)-1-isopropyl-1H-pyrazole-3-sulfonamide (Intermediate P2; 0.020 g, 0.081 mmol) and N,N-dimethylaminopyridine (0.030 g, 0.244 mmol) were dissolved in dry acetonitrile (1 mL) at room temperature and stirred for 10 minutes, after which time the mixture had become homogeneous. Diphenyl carbonate (0.019 g, 0.089 mmol) was then added as a solid and the slightly turbid reaction mixture was stirred at room temperature overnight. This was repeated 4 times at different temperatures. The crude reaction mixtures were combined and added to 4-fluoro-2-isopropyl-6-(pyridin-3-yl)aniline (Intermediate A1; 36.4 mg, 0.158 mmol). The mixture was then heated to 70° C. for 2 hours, evaporated to dryness in vacuo and the brown residue obtained triturated with 1:4 ethyl acetate:dichloromethane (4 mL). The filtrate was then purified by preparative HPLC [Gilson apparatus, basic procedure (0.1% ammonium bicarbonate), basic Waters X-Bridge Prep-C18, 5 μm, 19×50 mm column, 5-95% acetonitrile in water with 10 mM ammonium bicarbonate) to afford the title compound (26 mg, 30%) as a white solid.

¹H NMR (DMSO-d₆) δ 10.91 (br s, 1H), 8.60-8.39 (m, 2H), 7.86 (s, 1H), 7.73 (dt, 1H), 7.36 (ddd, 1H), 7.21 (dd, 1H), 7.07 (dd, 1H), 6.44 (s, 1H), 4.80 (sept, 1H), 3.48 (s, 2H), 3.04-2.93 (m, 1H), 2.15 (s, 6H), 1.38 (d, 6H) and 1.09 (d, 6H).

LCMS m/z 503.6 (M+H)⁺ (ES⁺); 501.4 (M−H)⁻ (ES⁻).

### Example 42: 5-((Dimethylamino)methyl)-N-((4-fluoro-2-isopropyl-6-(pyrimidin-5-yl)phenyl)carbamoyl)-1-methyl-1H-pyrazole-3-sulfonamide

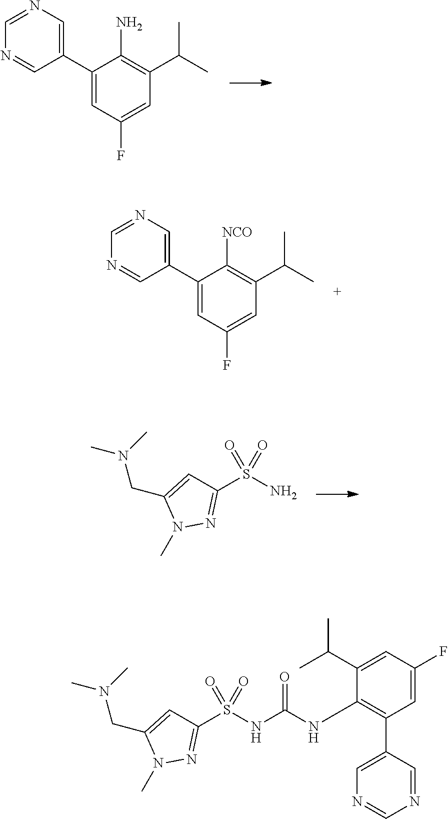

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-methyl-1H-pyrazole-3-sulfonamide (Intermediate P1) and 4-fluoro-2-isopropyl-6-(pyrimidin-5-yl)aniline (Intermediate A18) to afford the title compound (13.7 mg, 10%) as a colourless powder.

¹H NMR (DMSO-d6) δ 10.93 (s, 1H), 9.15 (s, 1H), 8.73 (s, 2H), 8.02 (s, 1H), 7.27 (dd, J=10.0, 3.0 Hz, 1H), 7.19 (dd, J=8.8, 3.0 Hz, 1H), 6.48 (s, 1H), 3.90 (s, 3H), 3.53 (s, 2H), 3.06 (hept, J=6.9 Hz, 1H), 2.19 (s, 6H), 1.11 (d, J=6.7 Hz, 6H).

LCMS m/z 476 (M+H)⁺ (ES⁺); 474 (M−H)⁻ (ES⁻).

### Example 43: 5-((Dimethylamino)methyl)-N-((4-fluoro-2-isopropyl-6-(pyrimidin-5-yl)phenyl)carbamoyl)-1-isopropyl-1H-pyrazole-3-sulfonamide

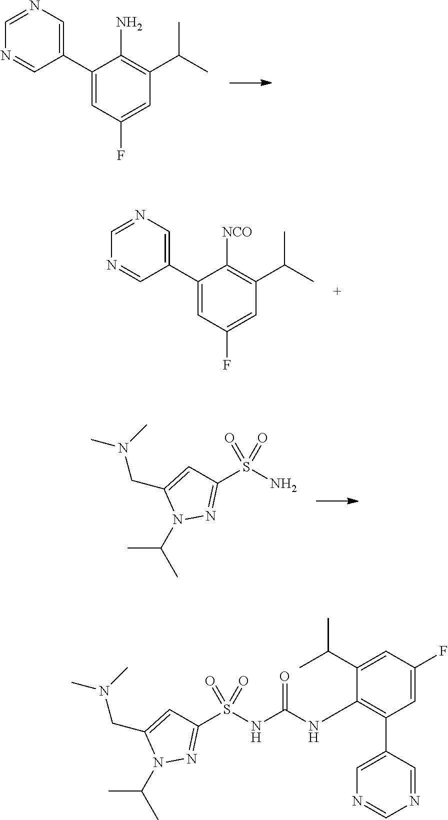

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-isopropyl-1H-pyrazole-3-sulfonamide (Intermediate P2) and 4-fluoro-2-isopropyl-6-(pyrimidin-5-yl)aniline (Intermediate A18) to afford the title compound (17.4 mg, 12%) as a colourless powder.

¹H NMR (DMSO-d6) δ 11.02 (s, 1H), 9.13 (s, 1H), 8.76 (s, 2H), 8.04 (s, 1H), 7.26 (dd, J=10.0, 3.0 Hz, 1H), 7.20 (dd, J=8.8, 3.0 Hz, 1H), 6.44 (s, 1H), 4.81 (sept, J=6.6 Hz, 1H), 3.51 (s, 2H), 3.03 (sept, J=7.0 Hz, 1H), 2.17 (s, 6H), 1.38 (d, J=6.6 Hz, 6H), 1.10 (d, J=6.8 Hz, 6H).

LCMS m/z 504 (M+H)⁺ (ES⁺); 502 (M−H)⁻ (ES⁻).

### Example 44: 5-((Dimethylamino)methyl)-N-((4-fluoro-2-isopropyl-6-(pyridin-3-yl)phenyl)carbamoyl)-1-methyl-1H-pyrazole-3-sulfonamide

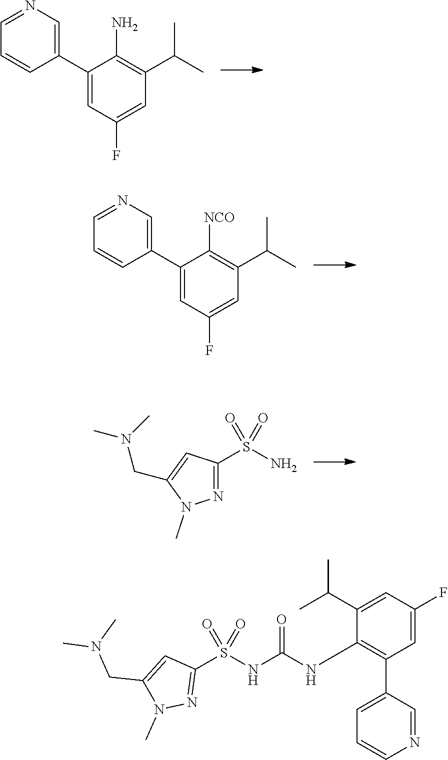

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-methyl-1H-pyrazole-3-sulfonamide (Intermediate P1) and 4-fluoro-2-isopropyl-6-(pyridin-3-yl)aniline (Intermediate A1) to afford the title compound (35.8 mg, 34%) as a colourless powder.

¹H NMR (DMSO-d6) δ 10.84 (s, 1H), 8.57-8.52 (m, 1H), 8.49 (s, 1H), 7.83 (s, 1H), 7.73-7.67 (m, 1H), 7.35 (dd, J=8.0, 4.9 Hz, 1H), 7.21 (dd, J=10.1, 3.0 Hz, 1H), 7.06 (dd, J=8.9, 3.0 Hz, 1H), 6.47 (s, 1H), 3.89 (s, 3H), 3.49 (s, 2H), 3.04 (sept, J=6.4 Hz, 1H), 2.17 (s, 6H), 1.10 (d, J=6.6 Hz, 6H).

LCMS m/z 475 (M+H)⁺ (ES⁺); 473 (M−H)⁻ (ES⁻).

### Example 45: N-((1,3-Dimethyl-1H-pyrazol-5-yl)-4-fluoro-6-isopropylphenyl)carbamoyl)-5-((dimethylamino)methyl)-1-methyl-1H-pyrazole-3-sulfonamide

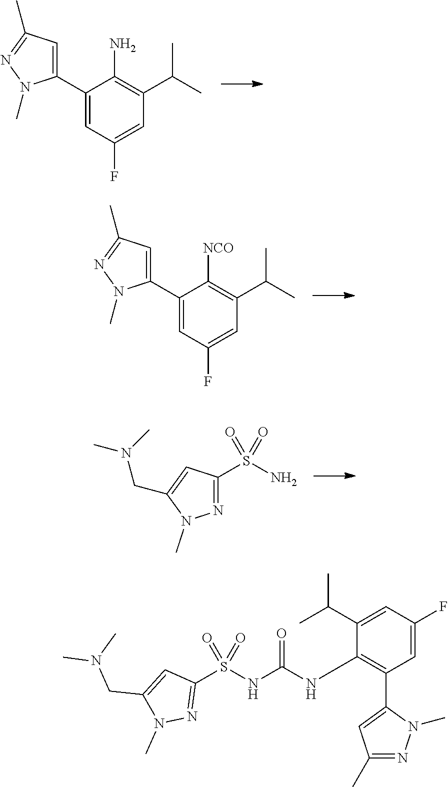

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-methyl-1H-Pyrazole-3-sulfonamide (Intermediate P1) and 2-(1,3-dimethyl-1H-pyrazol-5-yl)-4-fluoro-6-isopropylaniline (Intermediate A11) to afford the title compound (15.9 mg, 22%) as a colourless powder.

¹H NMR (DMSO-d6) δ 10.84 (s, 1H), 7.68 (s, 1H), 7.24 (dd, J=10.1, 3.0 Hz, 1H), 7.04 (dd, J=8.7, 3.0 Hz, 1H), 6.52 (s, 1H), 5.93 (s, 1H), 3.89 (s, 3H), 3.50 (s, 2H), 3.45 (s, 3H), 3.08-2.92 (m, 1H), 2.17 (s, 6H), 2.14 (s, 3H), 1.09 (d, J=6.8 Hz, 6H).

LCMS m/z 492 (M+H)⁺ (ES⁺); 490 (M−H)⁻ (ES⁻).

### Example 46: 5-((Dimethylamino)methyl)-N-((5-fluoro-3-isopropyl-[1,1′-biphenyl]-2-yl)carbamoyl)-1-isopropyl-1H-pyrazole-3-sulfonamide

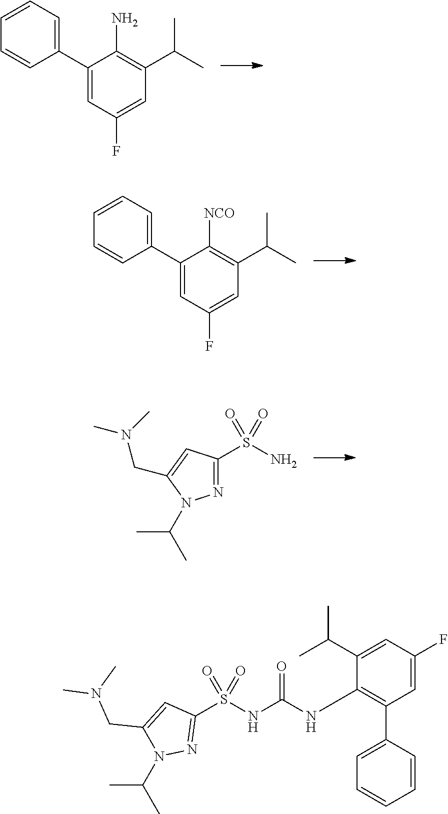

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-isopropyl-1H-pyrazole-3-sulfonamide (Intermediate P2) and 5-fluoro-3-isopropyl-[1,1′-biphenyl]-2-amine (Intermediate A4) to afford the title compound (35.8 mg, 32%) as a colourless powder.

¹H NMR (DMSO-d6) δ 10.77 (s, 1H), 7.70 (s, 1H), 7.39-7.27 (m, 5H), 7.16 (dd, J=10.1, 3.0 Hz, 1H), 6.97 (dd, J=8.9, 3.0 Hz, 1H), 6.51 (s, 1H), 4.83 (hept, J=6.6 Hz, 1H), 3.50 (s, 2H), 2.95 (hept, J=7.9 Hz, 1H), 2.17 (s, 6H), 1.39 (d, J=6.5 Hz, 6H), 1.09 (d, J=6.8 Hz, 6H).

LCMS m/z 502 (M+H)⁺ (ES⁺); 500 (M−H)⁻ (ES⁻).

### Example 47: N-((4-Fluoro-2-isopropyl-6-(2-(trifluoromethyl)pyridin-4-yl)phenyl)carbamoyl)-1-isopropyl-1H-pyrazole-3-sulfonamide

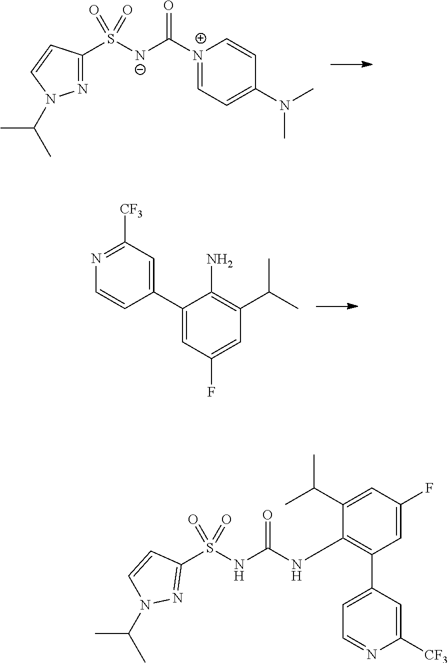

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl) ((1-isopropyl-1H-pyrazol-3-yl)sulfonyl)amide (Intermediate P3) and 4-fluoro-2-isopropyl-6-(2-(trifluoromethyl)pyridin-4-yl)aniline (Intermediate A31) to afford the title compound (21.9 mg, 28%) as a colourless powder.

¹H NMR (DMSO-d6) δ 11.04 (s, 1H), 8.70 (d, J=5.0 Hz, 1H), 8.04 (s, 1H), 7.89 (s, 1H), 7.88 (d, J=2.3 Hz, 1H), 7.64 (d, J=4.6 Hz, 1H), 7.28 (dd, J=9.9, 3.0 Hz, 1H), 7.19 (dd, J=8.8, 3.0 Hz, 1H), 6.48 (s, 1H), 4.57 (sept, J=6.5 Hz, 1H), 3.06 (sept, J=6.4 Hz, 1H), 1.42 (d, J=6.7 Hz, 6H), 1.10 (d, J=6.8 Hz, 6H).

LCMS m/z 514 (M+H)⁺ (ES⁺); 512 (M−H)⁻ (ES⁻).

### Example 48: N-((2-(2-Cyanopyridin-4-yl)-4-fluoro-6-isopropylphenyl) carbamoyl)-5-((dimethylamino)methyl)-1-methyl-1H-pyrazole-3-sulfonamide

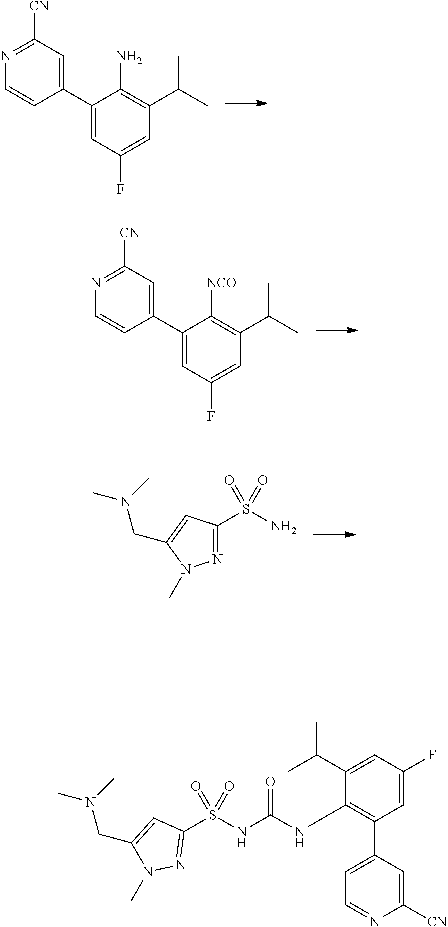

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-methyl-1H-pyrazole-3-sulfonamide (Intermediate P1) and 4-(2-amino-5-fluoro-3-isopropylphenyl)picolino-nitrile (Intermediate A28) to afford the title compound (7.9 mg, 7%) as a colourless powder.

¹H NMR (DMSO-d6) δ 10.91 (s, 1H), 8.66 (d, J=5.1 Hz, 1H), 8.07-7.97 (m, 2H), 7.73-7.61 (m, 1H), 7.27 (dd, J=9.9, 3.0 Hz, 1H), 7.15 (dd, J=8.8, 2.9 Hz, 1H), 6.33 (s, 1H), 3.86 (s, 3H), 3.48 (s, 2H), 3.12 (sept, J=6.5 Hz, 1H), 2.17 (s, 6H), 1.12 (d, J=6.8 Hz, 6H).

LCMS m/z 500.5 (M+H)⁺ (ES⁺); 498.4 (M−H)⁻ (ES⁻).

### Example 49: N-((2-(2-Cyanopyridin-4-yl)-4-fluoro-6-isopropylphenyl) carbamoyl)-5-((dimethylamino)methyl)-1-ethyl-1H-pyrazole-3-sulfonamide

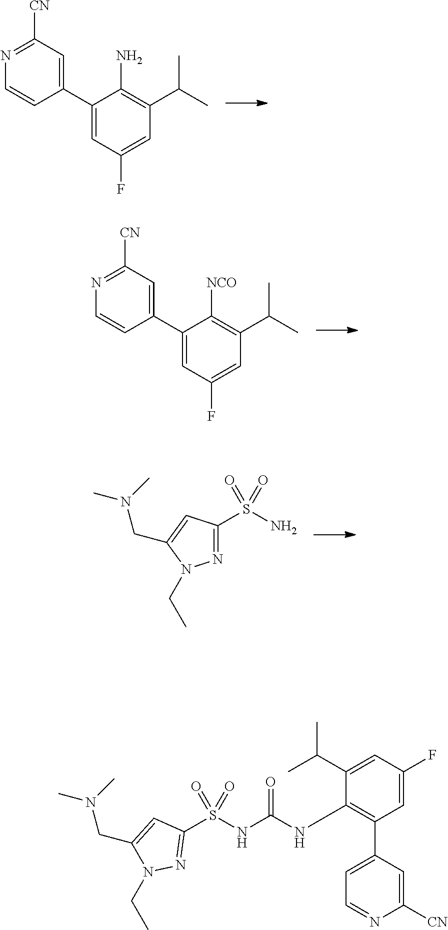

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-((dimethylamino)methyl)-1-ethyl-1H-pyrazole-3-sulfonamide (Intermediate P7) and 4-(2-amino-5-fluoro-3-isopropylphenyl)picolino-nitrile (Intermediate A28) to afford the title compound (6.9 mg, 6%) as a colourless powder.

¹H NMR (DMSO-d6) δ 8.65 (d, J=5.0 Hz, 1H), 8.03 (d, J=1.7 Hz, 1H), 7.97 (s, 1H), 7.73-7.65 (m, 1H), 7.26 (dd, J=10.0, 3.0 Hz, 1H), 7.15 (dd, J=8.8, 3.0 Hz, 1H), 6.29 (s, 1H), 4.17 (q, J=7.2 Hz, 2H), 3.46 (s, 2H), 3.21-3.02 (m, 1H), 2.16 (s, 6H), 1.33 (t, J=7.2 Hz, 3H), 1.11 (d, J=6.8 Hz, 6H).

LCMS m/z 514.6 (M+H)⁺ (ES⁺); 512.4 (M−H)⁻ (ES⁻).

### Example 50: N-((2-(2-(Dimethylamino)pyridin-4-yl)-4-fluoro-6-isopropylphenyl)carbamoyl)-1-isopropyl-1H-pyrazole-3-sulfonamide

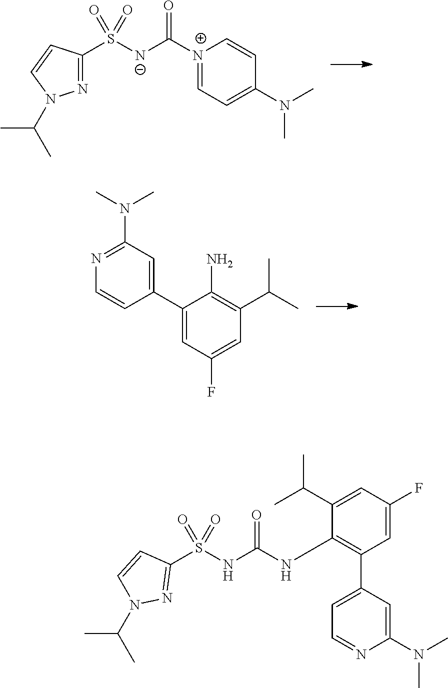

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl) ((1-isopropyl-1H-pyrazol-3-yl)sulfonyl)amide (Intermediate P3) and 4-(2-amino-5-fluoro-3-isopropylphenyl)-N,N-dimethylpyridin-2-amine (Intermediate A32) to afford the title compound (23.7 mg, 32%) as a colourless powder.

¹H NMR (DMSO-d6) δ 8.01 (d, J=5.1 Hz, 1H), 7.85 (s, 1H), 7.62 (s, 1H), 7.15 (dd, J=10.1, 3.0 Hz, 1H), 6.98 (dd, J=9.0, 2.9 Hz, 1H), 6.58 (s, 1H), 6.54-6.43 (m, 2H), 4.56 (sept, J=6.7 Hz, 1H), 3.02 (s, 6H), 3.02 (m, 1H), 1.43 (d, J=6.7 Hz, 6H), 1.07 (d, J=6.8 Hz, 6H), one exchangeable proton not visible.

LCMS m/z 489.6 (M+H)⁺ (ES⁺); 487.5 (M−H)⁻ (ES⁻).

### Example 51: N-((4-Fluoro-2-isopropyl-6-(2-(prop-1-yn-1-yl)pyridin-4-yl)phenyl)carbamoyl)-1-isopropyl-1H-pyrazole-3-sulfonamide

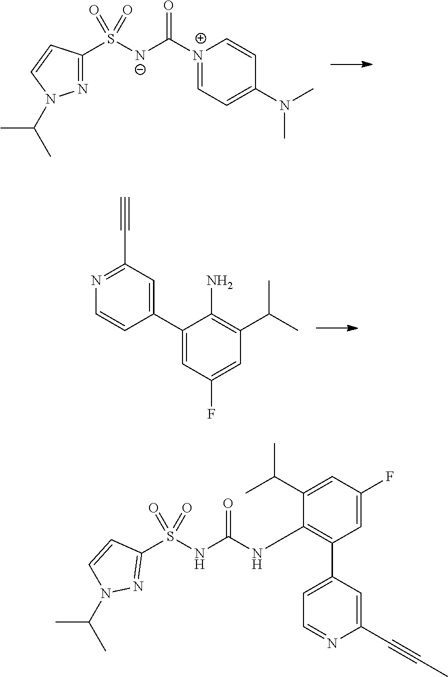

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl) ((1-isopropyl-1H-pyrazol-3-yl)sulfonyl)amide (Intermediate P3) and 4-fluoro-2-isopropyl-6-(2-(prop-1-yn-1-yl)pyridin-4-yl)aniline (Intermediate A33) to afford the title compound (21.2 mg, 29%) as a colourless powder.

¹H NMR (DMSO-d6) δ 10.94 (br s, 1H), 8.41 (d, J=5.1 Hz, 1H), 7.86 (s, 2H), 7.41 (s, 1H), 7.28-7.23 (m, 1H), 7.21 (dd, J=10.0, 3.0 Hz, 1H), 7.05 (dd, J=8.8, 2.9 Hz, 1H), 6.46 (s, 1H), 4.56 (sept, J=6.7 Hz, 1H), 3.12-2.95 (m, 1H), 2.09 (s, 3H), 1.43 (d, J=6.7 Hz, 6H), 1.09 (d, J=6.8 Hz, 6H).

LCMS m/z 484.4 (M+H)⁺ (ES⁺); 482.3 (M−H)⁻ (ES⁻).

### Example 52: N-((4-Fluoro-2-isopropyl-6-(2-methoxypyridin-4-yl)phenyl)carbamoyl)-5-(3-methoxyoxetan-3-yl)-1-methyl-1H-pyrazole-3-sulfonamide

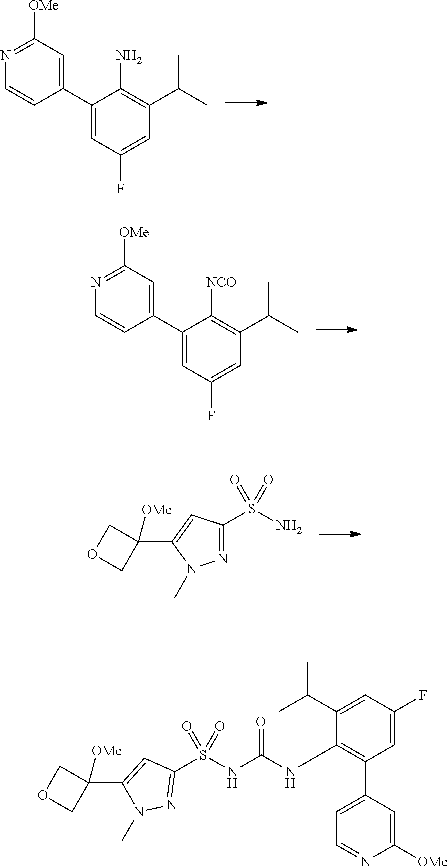

Prepared according to the general procedure of 5-((dimethylamino)methyl)-N-((4-fluoro-2-iso-propyl-6-(2-methoxypyridin-4-yl)phenyl)carbamoyl)-1-methyl-1H-pyrazole-3-sulfonamide (Example 36) from 5-(3-methoxyoxetan-3-yl)-1-methyl-1H-pyrazole-3-sulfonamide (Intermediate P8) and 4-fluoro-2-isopropyl-6-(2-methoxypyridin-4-yl)aniline (Intermediate A12) to afford the title compound (22.5 mg, 22%) as a colourless powder.

¹H NMR (DMSO-d6) δ 11.08 (s, 1H), 8.12 (d, J=5.3 Hz, 1H), 7.89 (s, 1H), 7.23 (dd, J=10.1, 2.9 Hz, 1H), 7.05 (dd, J=8.8, 2.9 Hz, 1H), 6.99 (s, 1H), 6.92 (dd, J=5.4, 1.5 Hz, 1H), 6.79 (s, 1H), 4.86 (d, J=7.4 Hz, 2H), 4.79 (d, J=7.3 Hz, 2H), 3.75 (s, 3H), 3.34 (s, 3H), 3.04 (sept, J=7.0 Hz, 1H), 2.95 (s, 3H), 1.09 (br s, 6H).

LCMS m/z 534.4 (M+H)⁺ (ES⁺); 532.2 (M−H) (ES⁻).

### Example 53: N-((4-Fluoro-2-isopropyl-6-(2-methoxypyridin-4-yl)phenyl) carbamoyl)-1-isopropyl-1H-imidazole-4-sulfonamide

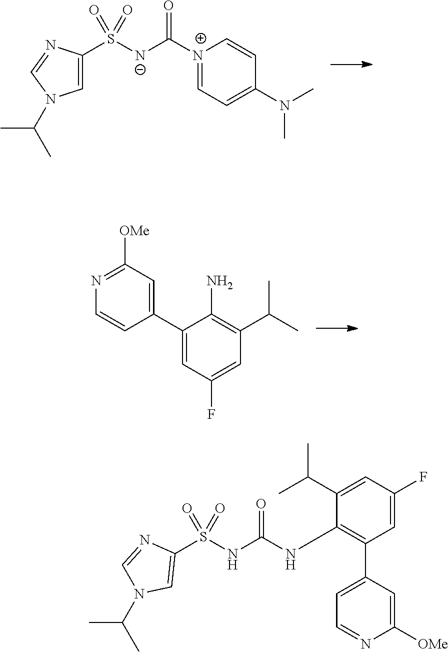

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl)((1-isopropyl-1H-imidazol-4-yl)sulfonyl)amide (Intermediate P6) and 4-fluoro-2-isopropyl-6-(2-methoxypyridin-4-yl)aniline (Intermediate A12) to afford the title compound (20 mg, 28%) as a white solid.

¹H NMR (DMSO-d6) δ 10.55 (bs, 1H), 8.09 (d, J=5.3 Hz, 1H), 7.95 (s, 1H), 7.90 (s, 1H), 7.80 (s, 1H), 7.21 (dd, J=10.0, 3.0 Hz, 1H), 7.03 (dd, J=8.9, 3.0 Hz, 1H), 6.83 (d, J=5.3 Hz, 1H), 6.74 (s, 1H), 4.48 (sept, J=6.1 Hz, 1H), 3.88 (s, 3H), 3.02-2.93 (m, 1H), 1.41 (d, J=6.7 Hz, 6H), 1.16-0.95 (m, 6H).

LCMS m/z 476.6 (M+H)⁺ (ES⁺).

### Example 54: N-((2-(2-Cyanopyridin-4-yl)-4-fluoro-6-isopropylphenyl) carbamoyl)-1-isopropyl-1H-imidazole-4-sulfonamide

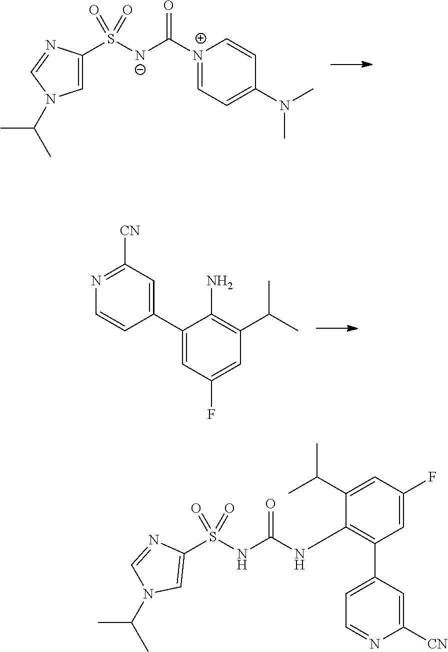

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl) ((1-isopropyl-1H-imidazol-4-yl)sulfonyl)amide (Intermediate P6) and 4-(2-amino-5-fluoro-3-isopropylphenyl)picolino-nitrile (Intermediate A28) to afford the title compound (19 mg, 27%) as a white solid.

¹H NMR (DMSO-d6) δ 10.78 (bs, 1H), 8.68 (d, J=5.1 Hz, 1H), 8.02 (s, 2H), 7.89 (s, 1H), 7.82 (s, 1H), 7.63 (d, J=5.0 Hz, 1H), 7.28 (dd, J=10.1, 3.0 Hz, 1H), 7.16 (dd, J=8.8, 3.0 Hz, 1H), 4.46 (sept, J=6.9 Hz, 1H), 3.13-3.01 (m, 1H), 1.41 (d, J=6.7 Hz, 6H), 1.10 (d, J=6.2 Hz, 6H).

LCMS m/z 471.2 (M+H)⁺ (ES⁺).

### Example 55: N-((7-Fluoro-5-(2-methoxypyridin-4-yl)-2,3-dihydro-1H-inden-4-yl)carbamoyl)-1-isopropyl-1H-pyrazole-3-sulfonamide

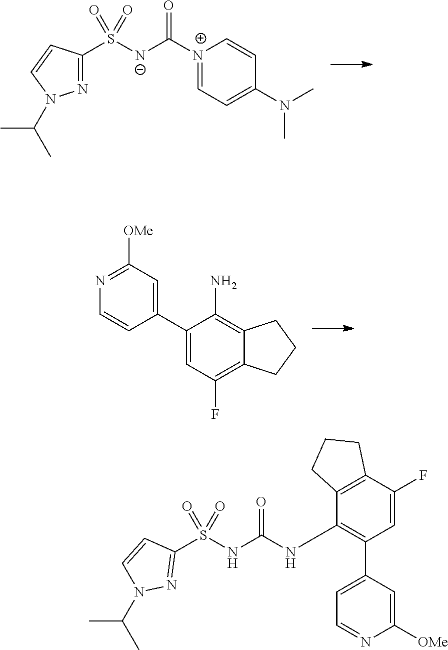

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl)((1-isopropyl-1H-pyrazol-3-yl)sulfonyl)amide (Intermediate P3) and 7-fluoro-5-(2-methoxypyridin-4-yl)-2,3-dihydro-1H-inden-4-amine (Intermediate A34) to afford the title compound (23.7 mg, 34%) as a colourless powder.

¹H NMR (DMSO-d6) δ 10.92 (s, 1H), 8.14 (d, J=5.3 Hz, 1H), 7.94 (d, J=2.4 Hz, 1H), 7.89 (s, 1H), 7.01 (d, J=9.2 Hz, 1H), 6.89 (dd, J=5.3, 1.5 Hz, 1H), 6.75 (s, 1H), 6.61 (s, 1H), 4.60 (sept, J=6.7 Hz, 1H), 3.89 (s, 3H), 2.94 (t, J=7.4 Hz, 2H), 2.66 (t, J=7.5 Hz, 2H), 2.03 (p, J=7.5 Hz, 2H), 1.44 (d, J=6.7 Hz, 6H).

LCMS m/z 474.4 (M+H)⁺ (ES⁺); 472.3 (M−H)⁻ (ES⁻).

### Example 56: 1-Isopropyl-N-((5-(2-methoxypyridin-4-yl)-2,3-dihydro-1H-inden-4-yl)carbamoyl)-1H-pyrazole-3-sulfonamide

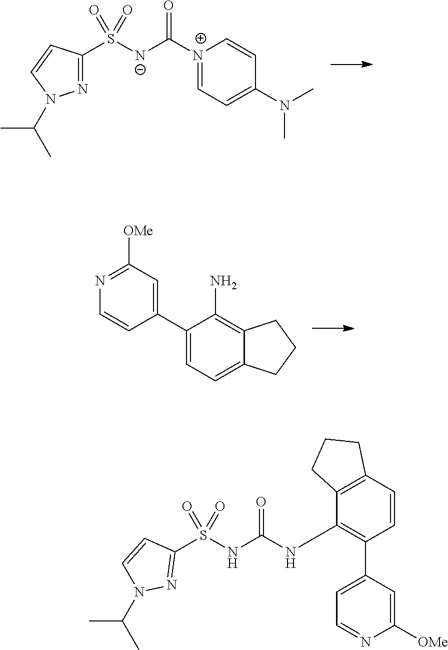

Prepared according to the general procedure for N-((4-fluoro-2-iso-propyl-6-(pyridin-3-yl)phenyl)carbamoyl)-1-iso-propyl-1H-pyrazole-3-sulfonamide (Example 1) from (4-(dimethylamino)pyridin-1-ium-1-carbonyl) ((1-isopropyl-1H-pyrazol-3-yl)sulfonyl)amide (Intermediate P3) and 5-(2-methoxypyridin-4-yl)-2,3-dihydro-1H-inden-4-amine (Intermediate A35) to afford the title compound (20.7 mg, 30%) as a colourless powder.

¹H NMR (DMSO-d6) δ 10.86 (s, 1H), 8.12 (d, J=5.4 Hz, 1H), 7.94 (d, J=2.3 Hz, 1H), 7.90 (s, 1H), 7.21 (d, J=7.7 Hz, 1H), 7.11 (d, J=7.6 Hz, 1H), 6.87 (dd, J=5.3, 1.4 Hz, 1H), 6.72 (s, 1H), 6.62 (s, 1H), 4.60 (sept, J=6.3 Hz, 1H), 3.88 (s, 3H), 20.91 (t, J=7.4 Hz, 2H), 2.62 (t, J=7.4 Hz, 2H), 1.97 (p, J=7.4 Hz, 2H), 1.44 (d, J=6.7 Hz, 6H).

LCMS m/z 456.4 (M+H)⁺ (ES⁺); 454.3 (M−H)⁻ (ES⁻).

### Example 57: 1-(2-(Dimethylamino)ethyl)-N-((4-fluoro-2-isopropyl-6-(pyridin-3-yl)phenyl)carbamoyl)-1H-pyrazole-3-sulfonamide

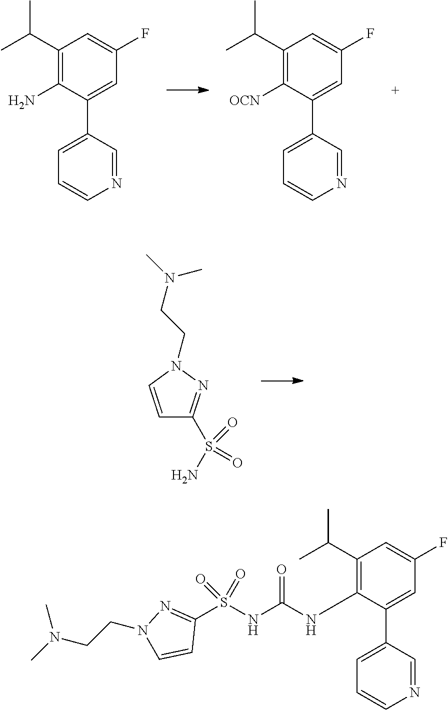

To a solution of 4-fluoro-2-isopropyl-6-(pyridin-3-yl)aniline (Intermediate A1) (0.5 g, 2.17 mmol, 1 eq) and triethylamine (439 mg, 4.34 mmol, 604.43 μL, 2 eq) in THF (10 mL) was added triphosgene (257 mg, 868.51 μmol, 0.4 eq) in portions at 5° C. Then the reaction mixture was heated to 70° C. and stirred for 1 hour. The reaction mixture was concentrated in vacuo. The residue was dissolved in EtOAc (100 mL) and the resulting mixture was filtered. The filtrate was concentrated in vacuo to give 3-(5-fluoro-2-isocyanato-3-isopropylphenyl)pyridine (0.2 g, crude) as a yellow oil. To a solution of 1-(2-(dimethylamino)ethyl)-1H-pyrazole-3-sulfonamide (Intermediate P9) (100 mg, 458.14 μmol, 1 eq) in THF (10 mL) was added MeONa (29 mg, 549.76 μmol, 1.2 eq) and the previously prepared 3-(5-fluoro-2-isocyanato-3-isopropylphenyl)pyridine (129 mg, 503.95 μmol, 1.1 eq). Then the solution was stirred at 70° C. for 20 minutes. The reaction mixture was concentrated in vacuo. The residue was purified by preparative reversed phase HPLC (see “Experimental Methods”, preparative reversed phase HPLC method 3) to give the title compound (19.52 mg, 40.72 μmol, 9% yield, 99% purity) as a yellow solid.

¹H NMR (DMSO-d₆) δ 8.51-8.48 (m, 2H), 7.70 (s, 2H), 7.49 (s, 1H), 7.28-7.26 (m, 1H), 7.10 (dd, 1H), 6.97 (dd, 1H), 6.28 (s, 1H), 4.20 (t, 2H), 3.14-3.12 (m, 1H), 2.67-2.62 (m, 2H), 2.18 (s, 6H) and 1.08 (dd, 6H).

LCMS: m/z 475 (M+H)⁺ (ES⁺).

### Example 58: 3-(Diethylamino)-N-((4-fluoro-2-isopropyl-6-(pyridin-3-yl) phenyl)carbamoyl)propane-1-sulfonamide

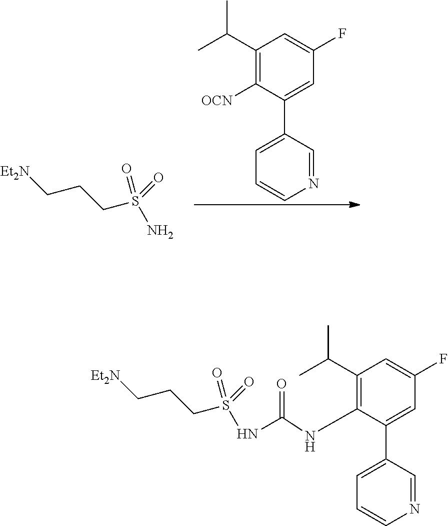

To a solution of 3-(diethylamino)propane-1-sulfonamide (Intermediate P32) (200 mg, 1.03 mmol, 1 eq) in THF (5 mL) was added NaOMe (56 mg, 1.03 mmol, 1 eq) and 3-(5-fluoro-2-isocyanato-3-isopropylphenyl)pyridine (Intermediate A41) (263.80 mg, 1.03 mmol, 1 eq). The reaction mixture was stirred at 70° C. for 30 minutes. The reaction mixture was filtered and the filtrate was concentrated in vacuo. The residue was purified by prep-HPLC (column: Phenomenex Gemini, 250 mm*25 mm*5 μm; mobile phase: [A: water (0.04% ammonium hydroxide v/v); B: MeCN]; B %: 18%-39%, 10 min) to give the title compound (58.2 mg, 11% yield, 100% purity on LCMS) as a brown solid.

¹H NMR (DMSO-d₆): δ 8.59 (br s, 1H), 8.50 (dd, 1H), 7.83-7.81 (m, 1H), 7.38 (dd 2H), 7.12 (dd, 1H), 6.97 (d, 1H), 3.29-3.25 (m, 1H), 2.75-2.73 (m, 2H), 2.49-2.43 (m, 6H), 1.64-1.60 (m, 2H), 1.16 (d, 6H) and 0.97 (t, 6H).

LCMS: m/z 451.2 (M+H)⁺ (ES⁺).

### Example 61: 1-(2-(Dimethylamino)ethyl)-N-((5-(2-methoxypyridin-4-yl)-2,3-dihydro-1H-inden-4-yl)carbamoyl)-1H-pyrazole-3-sulfonamide

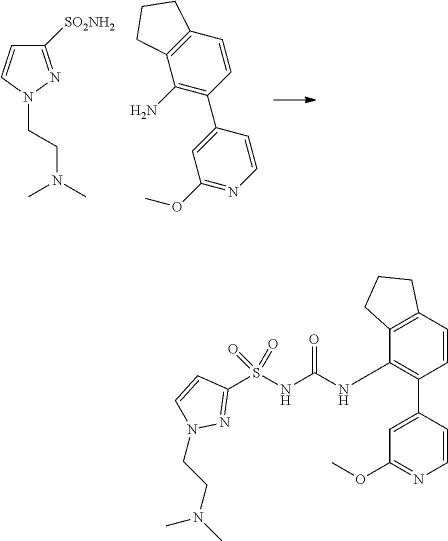 [SMILES: CN(C)CCn1ccc(S(N)(=O)=O)n1 ; COc1cc(-c2ccc3c(c2N)CCC3)ccn1 ; COc1cc(-c2ccc3c(c2NC(=O)NS(=O)(=O)c2ccn(CCN(C)C)n2)CCC3)ccn1]

5-(2-Methoxypyridin-4-yl)-2,3-dihydro-1H-inden-4-amine (Intermediate A35) (100 mg, 0.416 mmol) was dissolved in dry THF (5 mL). Triethylamine (70 μL, 0.502 mmol) was added, followed by a solution of bis(trichloromethyl) carbonate (123 mg, 0.416 mmol) in THF (1 mL). The slurry was stirred at room temperature for two hours before being filtered. The solid was washed with THF (5 mL) and DCM (5 mL) and then the filtrate was concentrated in vacua to give 4-(4-isocyanato-2,3-dihydro-1H-inden-5-yl)-2-methoxypyridine as a pale yellow solid that was used without further purification. 1-(2-(Dimethylamino)ethyl)-1H-pyrazole-3-sulfonamide (45 mg, 0.206 mmol) (Intermediate P9) was dissolved in dry THF (2 mL). Sodium tert-butoxide (2 M in THF) (104 μL, 0.208 mmol) was added and the mixture was stirred at room temperature for 30 minutes. A solution of 4-(4-isocyanato-2,3-dihydro-1H-inden-5-yl)-2-methoxypyridine (55 mg, 0.205 mmol) in DMF (2 mL) was added and the mixture was stirred overnight. The THF was removed in vacua. DMSO (1 mL) was added and the resulting solution was purified by reversed phase prep-HPLC (General Methods, basic prep) to afford the title compound (16 mg, 16%) as a colourless powder.

¹H NMR (DMSO-d6) δ 10.70 (br s, 1H), 8.12 (dd, J=5.3, 0.7 Hz, 1H), 7.88 (d, J=2.4 Hz, 1H), 7.86 (s, 1H), 7.20 (d, J=7.7 Hz, 1H), 7.11 (d, J=7.6 Hz, 1H), 6.87 (dd, J=5.3, 1.5 Hz, 1H), 6.73-6.71 (m, 1H), 6.58 (d, J=2.4 Hz, 1H), 4.31 (t, J=6.5 Hz, 2H), 3.89 (s, 3H), 2.91 (t, J=7.5 Hz, 2H), 2.75 (t, J=6.7 Hz, 2H), 2.67 (t, J=7.5 Hz, 2H), 2.23 (s, 6H), 1.99 (p, J=7.5 Hz, 2H).

LCMS; m/z 485.4 (M+H)+ (ES+); 483.3 (M−H)− (ES−).

### Example 64: 5-((Dimethylamino)methyl)-1-ethyl-N-((5-(2-methoxypyridin-4-yl)-2,3-dihydro-1H-inden-4-yl)carbamoyl)-1H-pyrazole-3-sulfonamide

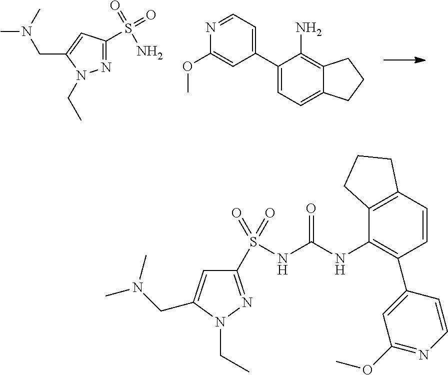 [SMILES: CCn1nc(S(N)(=O)=O)cc1CN(C)C ; COc1cc(-c2ccc3c(c2N)CCC3)ccn1 ; CCn1nc(S(=O)(=O)NC(=O)Nc2c(-c3ccnc(OC)c3)ccc3c2CCC3)cc1CN(C)C]

Prepared according to the general procedure of 1-(2-(dimethylamino)ethyl)-N-((5-(2-methoxypyridin-4-yl)-2,3-dihydro-1H-inden-4-yl)carbamoyl)-1H-pyrazole-3-sulfonamide (Example 61) from 5-((dimethylamino)methyl)-1-ethyl-1H-pyrazole-3-sulfonamide (Intermediate P7) and 5-(2-methoxypyridin-4-yl)-2,3-dihydro-1H-inden-4-amine (Intermediate A35) to afford the title compound (29 mg, 28%) as a colourless powder.

¹H NMR (DMSO-d6) δ 10.81 (s, 1H), 8.13 (dd, J=5.3, 0.7 Hz, 1H), 7.92 (s, 1H), 7.22 (d, J=7.7 Hz, 1H), 7.12 (d, J=7.6 Hz, 1H), 6.87 (dd, J=5.3, 1.5 Hz, 1H), 6.73-6.71 (m, 1H), 6.56 (s, 1H), 4.22 (q, J=7.2 Hz, 2H), 3.89 (s, 3H), 3.50 (s, 2H), 2.91 (t, J=7.5 Hz, 2H), 2.62 (t, J=7.5 Hz, 2H), 2.17 (s, 6H), 1.96 (p, J=7.5 Hz, 2H), 1.36 (t, J=7.2 Hz, 3H).

LCMS; m/z 499.4 (M+H)+ (ES+); 497.3 (M−H)− (ES−).

The compounds of examples 59, 60, 62, 63 and 65-69 were synthesised by methods analogous to those outlined above and below.

TABLE 1
¹H NMR and MS data

| Ex | Structure and Name | 1H NMR spectrum | MS | MW |
|---|---|---|---|---|
| 59 | 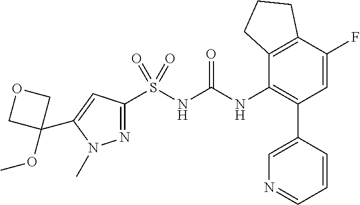 [SMILES: COC1(c2cc(S(=O)(=O)NC(=O)Nc3c(-c4cccnc4)cc(F)c4c3CCC4)nn2C)COC1] N-((7-Fluoro-5-(pyridin-3-yl)-2,3- | 1H NMR (400 MHz, DMSO-d6) δ 10.94 (s, 1H), 8.56 (dd, J = 4.8, 1.7 Hz, 1H), 8.48 (s, 1H), 7.84 (s, 1H), 7.75-7.70 (m, 1H), 7.43 (dd, J = 7.9, 4.9 Hz, 1H), 7.02 (d, J = 9.2 Hz, 1H), 6.96 (s, 1H), 4.89 (d, J = 7.3 Hz, 2H), 4.80 (d, J = 7.4 Hz, 2H), 3.76 (s, 3H), 2.98 (s, 3H) 2.95 (t, J = 7.5 Hz, 2H), 2.71 (t, J = 7.5 Hz, 2H), 2.04 (p, J = 7.6 Hz, 2H). | m/z 502.4 (M + H)+ (ES+); 500.3 (M − H)− (ES−) | 501.53 |
| | dihydro-1H-inden-4-yl)carbamoyl)-5- | | | |
| | (3-methoxyoxetan-3-yl)-1-methyl-1H- | | | |
| | pyrazole-3-sulfonamide | | | |
| 60 | 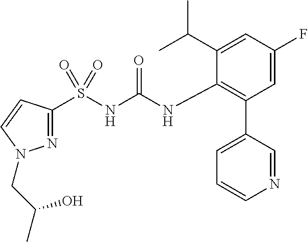 [SMILES: CC(C)c1cc(F)cc(-c2cccnc2)c1NC(=O)NS(=O)(=O)c1ccn(C[C@@H](C)O)n1] | 1H NMR (400 MHz, DMSO-d6) δ 8.52-8.48 (m, 2H), 7.75-7.70 (m, 3H), 7.34-7.32 (m, 1H), 7.18-7.15 (m, 1H), 7.03- 7.02 (m, 1H), 6.43 (s, 1H), 5.05 (s, 1H), 4.11-3.96 (m, 3H), 3.07-3.05 (m, 1H), 1.09 (d, J = 6.4 Hz, 6H), 1.04 (d, J = 6.4 Hz, 3H). | m/z 462.2 (M + H)+ | 461.51 |
| | (R)-N-((4-Fluoro-2-isopropyl-6- | | | |
| | (pyridin-3-yl)phenyl)carbamoyl)-1-(2- | | | |
| | hydroxypropyl)-1H-pyrazole-3- | | | |
| | sulfonamide | | | |
| 61 | 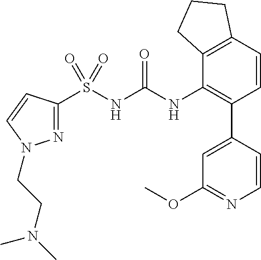 [SMILES: COc1cc(-c2ccc3c(c2NC(=O)NS(=O)(=O)c2ccn(CCN(C)C)n2)CCC3)ccn1] 1-(2-(Dimethylamino)ethyl)-N-((5-(2- | 1H NMR (500 MHz, DMSO-d6) δ 10.70 (br s, 1H), 8.12 (dd, J = 5.3, 0.7 Hz, 1H), 7.88 (d, J = 2.4 Hz, 1H), 7.86 (s, 1H), 7.20 (d, J = 7.7 Hz, 1H), 7.11 (d, J = 7.6 Hz, 1H), 6.87 (dd, J = 5.3, 1.5 Hz, 1H), 6.73-6.71 (m, 1H), 6.58 (d, J = 2.4 Hz, 1H), 4.31 (t, J = 6.5 Hz, 2H), 3.89 (s, 3H), 2.91 (t, J = 7.5 Hz, 2H), 2.75 (t, J = 6.7 Hz, 2H), 2.67 (t, J = 7.5 Hz, 2H), 2.23 (s, 6H), 1.99 (p, J = 7.5 Hz, 2H). | m/z 485.4 (M + H)+ (ES+); 483.3 (M − H)− (ES−) | 484.57 |
| | methoxypyridin-4-yl)-2,3-dihydro-1H- | | | |
| | inden-4-yl)carbamoyl)-1H-pyrazole-3- | | | |
| | sulfonamide | | | |
| 62 | 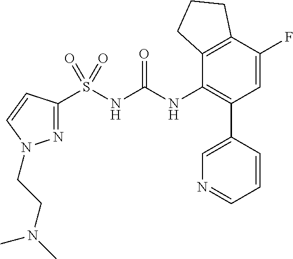 [SMILES: CN(C)CCn1ccc(S(=O)(=O)NC(=O)Nc2c(-c3cccnc3)cc(F)c3c2CCC3)n1] 1-(2-(Dimethylamino)ethyl)-N-((7- | 1H NMR (400 MHz, DMSO-d6) δ 8.55 (dd, J = 4.8, 1.7 Hz, 1H), 8.50 (d, J = 2.3 Hz, 1H), 7.88 (d, J = 2.3 Hz, 1H), 7.83 (s, 1H), 7.73-7.68 (m, 1H), 7.40 (dd, J = 7.9, 4.9 Hz, 1H), 7.02 (d, J = 9.2 Hz, 1H), 6.55 (d, J = 2.3 Hz, 1H), 4.33 (t, J = 6.5 Hz, 2H), 2.96 (t, J = 7.4 Hz, 2H), 2.79 (t, J = 6.4 Hz, 2H), 2.73 (t, J = 7.5 Hz, 2H), 2.26 (s, 6H), 2.06 (p, J = 7.4 Hz, 2H); NH not observed. | m/z 473.4 (M + H)+ (ES+); 471.0 (M − H)− (ES−) | 472.54 |
| | fluoro-5-(pyridin-3-yl)-2,3-dihydro-1H- | | | |
| | inden-4-yl)carbamoyl)-1H-pyrazole-3- | | | |
| | sulfonamide | | | |
| 63 | 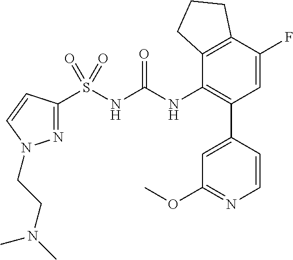 [SMILES: COc1cc(-c2cc(F)c3c(c2NC(=O)NS(=O)(=O)c2ccn(CCN(C)C)n2)CCC3)ccn1] | 1H NMR (400 MHz, DMSO-d6) δ 10.60 (s, 1H), 8.13 (d, J = 5.3 Hz, 1H), 7.88 (d, J = 2.3 Hz, 1H), 7.85 (s, 1H), 7.00 (d, J = 9.2 Hz, 1H), 6.90 (dd, J = 5.3, 1.5 Hz, 1H), 6.76 (d, J = 1.4 Hz, 1H), 6.56 (d, J = 2.3 Hz, 1H), 4.32 (t, J = 6.5 Hz, 2H), 3.89 (s, 3H), 2.95 (t, J = 7.4 Hz, 2H), 2.80 (t, J = 6.5 Hz, 2H), 2.71 (t, J = 7.5 Hz, 2H), 2.26 (s, 6H), 2.05 (p, J = 7.6 Hz, 2H). | m/z 503.5 (M + H)+ (ES+); 501.2 (M − H)− (ES−) | 502.56 |
| | 1-(2-(Dimethylamino)ethyl)-N-((7- | | | |
| | fluoro-5-(2-methoxypyridin-4-yl)-2,3- | | | |
| | dihydro-1H-inden-4-yl)carbamoyl)-1H- | | | |
| | pyrazole-3-sulfonamide | | | |
| 64 | 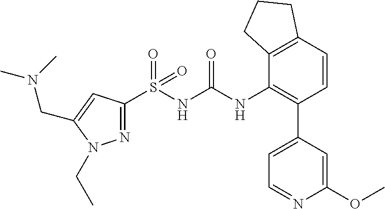 5-((Dimethylamino)methyl)-1-ethyl-N- | 1H NMR (500 MHz, DMSO-d6) δ 10.81 (s, 1H), 8.13 (dd, J = 5.3, 0.7 Hz, 1H), 7.92 (s, 1H), 7.22 (d, J = 7.7 Hz, 1H), 7.12 (d, J = 7.6 Hz, 1H), 6.87 (dd, J = 5.3, 1.5 Hz, 1H), 6.73-6.71 (m, 1H), 6.56 (s, 1H), 4.22 (q, J = 7.2 Hz, 2H), 3.89 (s, 3H), 3.50 (s, 2H), 2.91 (t, J = 7.5 Hz, 2H), 2.62 (t, J = 7.5 Hz, 2H), 2.17 (s, 6H), 1.96 (p, J = 7.5 Hz, 2H), 1.36 (t, J = 7.2 Hz, 3H). | m/z 499.4 (M + H)+ (ES+); 497.3 (M − H)− (ES−) | 498.6 |
| | ((5-(2-methoxypyridin-4-yl)-2,3- | | | |
| | dihydro-1H-inden-4-yl)carbamoyl)-1H- | | | |
| | pyrazole-3-sulfonamide | | | |
| 65 | 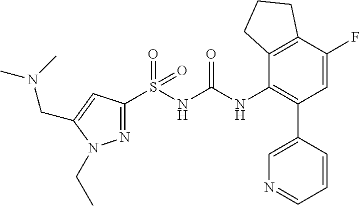 [SMILES: CCn1nc(S(=O)(=O)NC(=O)Nc2c(-c3cccnc3)cc(F)c3c2CCC3)cc1CN(C)C] 5-((Dimethylamino)methyl)-1-ethyl-N- | 1H NMR (400 MHz, DMSO-d6) δ 10.83 (s, 1H), 8.56 (dd, J = 4.8, 1.6 Hz, 1H), 8.50 (d, J = 2.2 Hz, 1H), 7.90 (s, 1H), 7.72- 7.68 (m, 1H), 7.43-7.39 (m, 1H), 7.03 (d, J = 9.2 Hz, 1H), 6.54 (s, 1H), 4.23 (q, J = 7.2 Hz, 2H), 3.51 (s, 2H), 2.95 (t, J = 7.4 Hz, 2H), 2.68 (t, J = 7.3 Hz, 2H), 2.18 (s, 6H), 2.03 (p, J = 7.6 Hz, 2H), 1.36 (t, J = 7.2 Hz, 3H). | m/z, 487.5 (M + H)+ (ES+); 485.3 (M − H)− (ES−) | 486.56 |
| | ((7-fluoro-5-(pyridin-3-yl)-2,3-dihydro- | | | |
| | 1H-inden-4-yl)carbamoyl)-1H-pyrazole- | | | |
| | 3-sulfonamide | | | |
| 66 | 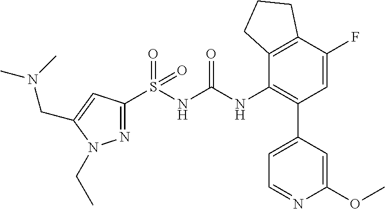 5-((Dimethylamino)methyl)-1-ethyl-N- | 1H NMR (400 MHz, DMSO-d6) δ 10.85 (s, 1H), 8.14 (d, J = 5.3 Hz, 1H), 7.90 (s, 1H), 7.02 (d, J = 9.2 Hz, 1H), 6.89 (dd, J = 5.3, 1.5 Hz, 1H), 6.75 (s, 1H), 6.56 (s, 1H), 4.22 (q, J = 7.2 Hz, 2H), 3.89 (s, 3H), 3.51 (s, 2H), 2.94 (t, J = 7.5 Hz, 2H), 2.66 (t, J = 7.1 Hz, 2H), 2.18 (s, 6H), 2.02 (p, J = 7.6 Hz, 2H), 1.35 (t, J = 7.2 Hz, 3H). | m/z 517.4 (M + H)+ (ES+); 515.3 (M − H)− (ES−) | 516.59 |
| | ((7-fluoro-5-(2-methoxypyridin-4-yl)- | | | |
| | 2,3-dihydro-1H-inden-4-yl)carbamoyl)- | | | |
| | 1H-pyrazole-3-sulfonamide | | | |
| 67 | 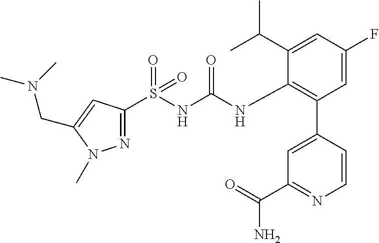 [SMILES: CC(C)c1cc(F)cc(-c2ccnc(C(N)=O)c2)c1NC(=O)NS(=O)(=O)c1cc(CN(C)C)n(C)n1] | 1H NMR (400 MHz, DMSO-d6) δ 10.86 (s, 1H), 8.53 (d, J = 5.0 Hz, 1H), 8.16 (d, J = 2.7 Hz, 1H), 7.96 (s, 2H), 7.69 (s, 1H), 7.49 (dd, J = 5.1, 1.8 Hz, 1H), 7.25 (dd, J = 10.1, 3.0 Hz, 1H), 7.10 (dd, J = 8.8, 2.9 Hz, 1H), 6.41 (s, 1H), 3.86 (s, 3H), 3.47 (s, 2H), 3.17-2.96 (m, 1H), 2.16 (s, 6H), 1.11 (br s, 6H). | m/z 518.4 (M + H)+ (ES+); 516.3 (M − H)− (ES−) | 517.58 |
| | 4-(2-(3-((5-((Dimethylamino)methyl)-1- | | | |
| | methyl-1H-pyrazol-3- | | | |
| | yl)sulfonyl)ureido)-5-fluoro-3- | | | |
| | isopropylphenyl)picolinamide | | | |
| 68 | 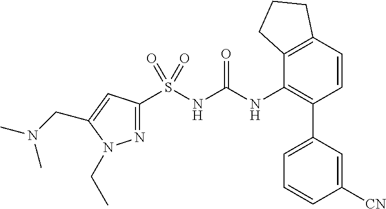 [SMILES: CCn1nc(S(=O)(=O)NC(=O)Nc2c(-c3cccc(C#N)c3)ccc3c2CCC3)cc1CN(C)C] N-((5-(3-Cyanophenyl)-2,3-dihydro-1H- | ¹H NMR (DMSO-d6) δ 10.82 (s, 1H), 7.94 (s, 1H), 7.82-7.79 (m, 1H), 7.73- 7.72 (m, 1H), 7.63-7.53 (m, 2H), 7.23 (d, J = 7.7 Hz, 1H), 7.13 (d, J = 7.7 Hz, 1H), 6.52 (s, 1H), 4.21 (q, J = 7.2 Hz, 2H), 3.49 (s, 2H), 2.92 (t, J = 7.4 Hz, 2H), 2.64 (t, J = 7.4 Hz, 2H), 2.17 (s, 6H), 1.98 (p, J = 7.5 Hz, 2H), 1.35 (t, J = 7.2 Hz, 3H). | m/z 493.0 (M + H)+ (ES+); 491.3 (M − H)− (ES−). | 492.6 |
| | inden-4-yl)carbamoyl)-5- | | | |
| | ((dimethylamino)methyl)-1-ethyl-1H- | | | |
| | pyrazole-3-sulfonamide | | | |
| 69 | 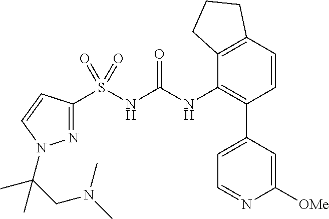 [SMILES: COc1cc(-c2ccc3c(c2NC(=O)NS(=O)(=O)c2ccn(C(C)(C)CN(C)C)n2)CCC3)ccn1] 1-(1-(Dimethylamino)-2-methylpropan- | 1H NMR (DMSO-d6) δ 10.84 (s, 1H), 8.15 (dd, J = 5.3, 0.7 Hz, 1H), 7.95 (d, J = 2.4 Hz, 1H), 7.92 (s, 1H), 7.22 (d, J = 7.7 Hz, 1H), 7.12 (d, J = 7.7 Hz, 1H), 6.89 (dd, J = 5.3, 1.5 Hz, 1H), 6.73-6.72 (m, 1H), 6.64 (d, J = 2.4 Hz, 1H), 3.89 (s, 3H), 2.91 (t, J = 7.5 Hz, 2H), 2.63 (t, J = 7.3 Hz, 2H), 2.58 (s, 2H), 1.97 (p, J = 7.6 Hz, 2H), 1.92 (s, 6H), 1.53 (s, 6H). | m/z 513.5 (M + H)+ (ES+) | 512.62 |
| | 2-yl)-N-((5-(2-methoxypyridin-4-yl)- | | | |
| | 2,3-dihydro-1H-inden-4-yl)carbamoyl)- | | | |
| | 1H-pyrazole-3-sulfonamide | | | |

### Example 70: 1-Isopropyl-N-((5-(2-methoxypyridin-4-yl)-2,3-dihydro-1H-inden-4-yl)carbamoyl)-6-oxo-1,6-dihydropyridine-3-sulfonamide

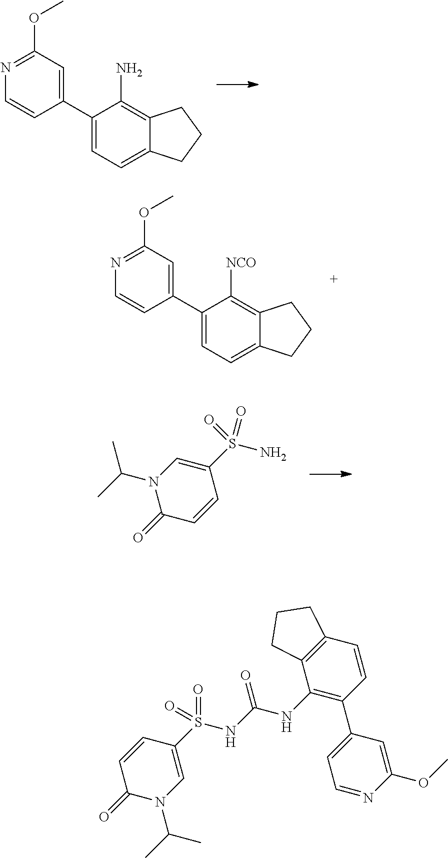

5-(2-Methoxypyridin-4-yl)-2,3-dihydro-1H-inden-4-amine (Intermediate A35) (0.30 g, 1.25 mmol) was dissolved in THF (10 mL). TEA (0.20 mL, 1.43 mmol) was added, followed by a solution of bis(trichloromethyl) carbonate (0.35 g, 1.18 mmol) in THF (2 mL). The mixture was stirred at room temperature for 1 hour, then concentrated in vacua and dried for 30 minutes to afford the intermediate 4-(4-isocyanato-2,3-dihydro-1H-inden-5-yl)-2-methoxypyridine as a pale yellow solid which was used without further purification.

1-Isopropyl-6-oxo-1,6-dihydropyridine-3-sulfonamide (Intermediate P12) (45 mg, 0.21 mmol) was dissolved in dry THF (2 mL). NaOtBu (2 M in THF) (0.125 ml, 0.250 mmol) was added and the mixture was stirred at room temperature for 1 hour. A solution of 4-(4-isocyanato-2,3-dihydro-1H-inden-5-yl)-2-methoxypyridine (prepared above) (55 mg) in THF (2 mL) was added and the mixture was stirred at room temperature overnight. The solvent was removed in vacuo and the residue was dissolved in DMSO (2 mL) and purified by basic prep-HPLC to afford the title compound (41 mg, 40%) as a colourless powder. ¹H NMR (DMSO-d6) δ 10.76 (s, 1H), 8.13 (d, J=2.6 Hz, 1H), 8.03 (dd, J=5.3, 0.7 Hz, 1H), 7.91 (s, 1H), 7.60 (dd, J=9.5, 2.6 Hz, 1H), 7.20 (d, J=7.7 Hz, 1H), 7.10 (d, J=7.6 Hz, 1H), 6.83 (dd, J=5.3, 1.5 Hz, 1H), 6.65 (s, 1H), 6.47 (d, J=9.6 Hz, 1H), 4.99 (sept, J=6.8 Hz, 1H), 3.84 (s, 3H), 2.91 (t, J=7.5 Hz, 2H), 2.67 (t, J=7.5 Hz, 2H), 1.98 (p, J=7.4 Hz, 2H), 1.29 (d, J=6.8 Hz, 6H).

LCMS: m/z 483.3 (M+H)⁺ (ES⁺); 481.5 (M−H)⁻ (ES⁻).

### Example 71: N-((5-(2-Cyanopyridin-4-yl)-2,3-dihydro-1H-inden-4-yl) carbamoyl)-1-isopropyl-6-oxo-1,6-dihydropyridine-3-sulfonamide, sodium salt

### Step A: N-((5-(2-Cyanopyridin-4-yl)-2,3-dihydro-1H-inden-4-yl) carbamoyl)-1-isopropyl-6-oxo-1,6-dihydropyridine-3-sulfonamide

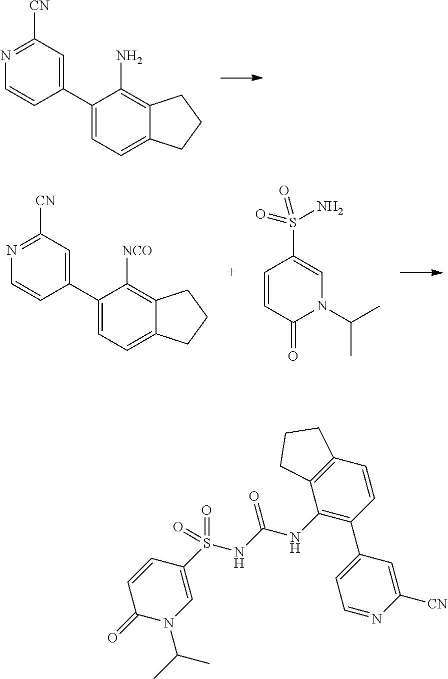

Prepared according to the general procedure of 1-isopropyl-N-((5-(2-methoxypyridin-4-yl)-2,3-dihydro-1H-inden-4-yl)carbamoyl)-6-oxo-1,6-dihydropyridine-3-sulfonamide (Example 70) from 4-(4-amino-2,3-dihydro-1H-inden-5-yl)picolinonitrile (Intermediate A36) (0.03 g, 0.123 mmol) and 1-isopropyl-6-oxo-1,6-dihydropyridine-3-sulfonamide (Intermediate P12) (0.027 g, 0.123 mmol) and purified by reversed phase flash C18 chromatography (12 g column, 0-60% MeCN/10 mM ammonium bicarbonate) to afford the title compound (35 mg, 30%) as a flocculent white solid.

¹H NMR (DMSO-d6) δ 8.56 (d, J=50.1 Hz, 1H), 7.93 (d, J=2.6 Hz, 1H), 7.89 (d, J=1.6 Hz, 1H), 7.75 (br s, 1H), 7.59 (dd, J=5.1, 1.8 Hz, 1H), 7.51 (dd, J=9.5, 2.5 Hz, 1H), 7.17-7.12 (m, 2H), 6.32 (d, J=9.4 Hz, 1H), 4.96 (sept, J=6.7 Hz, 1H), 2.91 (t, J=7.5 Hz, 2H), 2.74 (t, J=7.4 Hz, 2H), 1.98 (p, J=7.5 Hz, 2H), 1.25 (d, J=6.8 Hz, 6H). One exchangeable proton not observed.

LCMS: m/z 478.3 (M+H)⁺ (ES⁺); 476.2 (M−H)⁻ (ES⁻).

### Step B: N-((5-(2-Cyanopyridin-4-yl)-2,3-dihydro-1H-inden-4-yl) carbamoyl)-1-isopropyl-6-oxo-1,6-dihydropyridine-3-sulfonamide, sodium salt

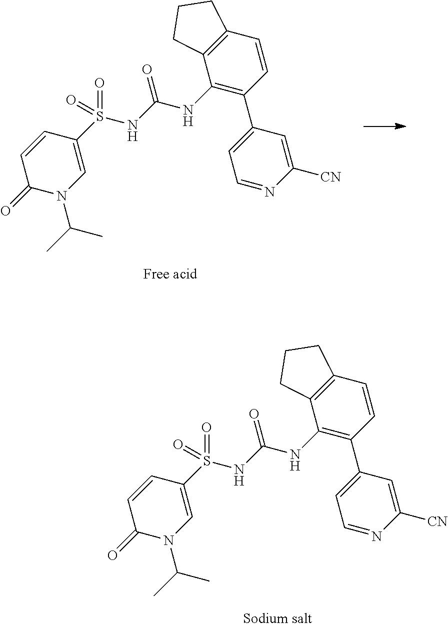 [SMILES: CC(C)n1cc(S(=O)(=O)NC(=O)Nc2c(-c3ccnc(C#N)c3)ccc3c2CCC3)ccc1=O ; CC(C)n1cc(S(=O)(=O)NC(=O)Nc2c(-c3ccnc(C#N)c3)ccc3c2CCC3)ccc1=O]

N-((5-(2-Cyanopyridin-4-yl)-2,3-dihydro-1H-inden-4-yl)carbamoyl)-1-isopropyl-6-oxo-1,6-dihydropyridine-3-sulfonamide (0.025 g, 0.052 mmol) was treated with 0.1 M NaOH solution (520 μL) and the resultant solution was freeze-dried to afford the title compound (26 mg, 99%) as a white solid.

¹H NMR (DMSO-d6) δ 8.54 (dd, J=5.1, 0.8 Hz, 1H), 7.91-7.89 (m, 1H), 7.87 (d, J=2.5 Hz, 1H), 7.60 (dd, J=5.1, 1.8 Hz, 1H), 7.54-7.46 (m, 2H), 7.13-7.09 (m, 2H), 6.27 (d, J=9.4 Hz, 1H), 4.97 (sept, J=6.7 Hz, 1H), 2.89 (t, J=7.5 Hz, 2H), 2.75 (t, J=7.4 Hz, 2H), 1.96 (p, J=7.5 Hz, 2H), 1.25 (d, J=6.8 Hz, 6H).

LCMS: m/z 478.3 (M+H)⁺ (ES⁺); 476.2 (M−H)⁻ (ES⁻).

### Example 72: N-((5-(2-Cyanopyridin-4-yl)-2,3-dihydro-1H-inden-4-yl) carbamoyl)-4-isopropyl-5-oxo-4,5-dihydropyrazine-2-sulfonamide, sodium salt

### Step A: N-((5-(2-Cyanopyridin-4-yl)-2,3-dihydro-1H-inden-4-yl) carbamoyl)-4-isopropyl-5-oxo-4,5-dihydropyrazine-2-sulfonamide

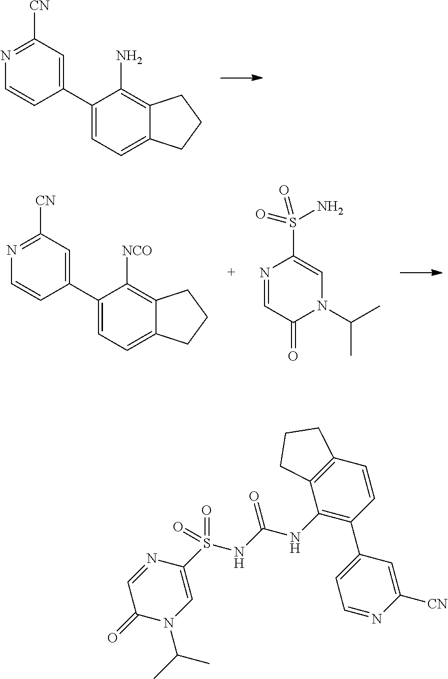

Prepared according to the general procedure of 1-isopropyl-N-((5-(2-methoxypyridin-4-yl)-2,3-dihydro-1H-inden-4-yl)carbamoyl)-6-oxo-1,6-dihydropyridine-3-sulfonamide (Example 70) from 4-(4-amino-2,3-dihydro-1H-inden-5-yl)picolinonitrile (Intermediate A36) (0.03 g, 0.123 mmol) and 4-isopropyl-5-oxo-4,5-dihydropyrazine-2-sulfonamide (Intermediate P13) (0.027 g, 0.123 mmol) and purified by reversed phase flash C18 chromatography (12 g column, 0-60% MeCN/10 mM ammonium bicarbonate) to afford the title compound (0.023 g, 19%) as a flocculent yellow solid.

¹H NMR (DMSO-d6) δ 8.58 (d, J=5.1 Hz, 1H), 7.93 (s, 2H), 7.89 (d, J=1.7 Hz, 1H), 7.76 (br s, 1H), 7.59 (dd, J=5.2, 1.7 Hz, 1H), 7.19-7.12 (m, 2H), 4.84 (p, J=6.8 Hz, 1H), 2.91 (t, J=7.5 Hz, 2H), 2.75 (t, J=7.4 Hz, 2H), 1.99 (p, J=7.5 Hz, 2H), 1.28 (d, J=6.8 Hz, 6H).

LCMS: m/z 479.3 (M+H)⁺ (ES⁺); 477.2 (M−H)⁻ (ES⁻).

### Step B: N-((5-(2-Cyanopyridin-4-yl)-2,3-dihydro-1H-inden-4-yl) carbamoyl)-4-isopropyl-5-oxo-4,5-dihydropyrazine-2-sulfonamide, sodium salt

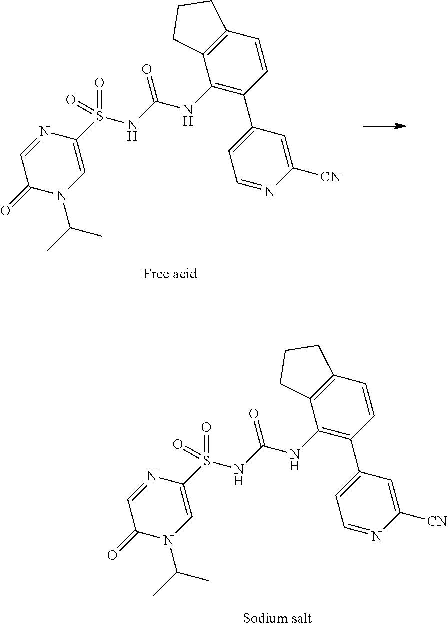 [SMILES: CC(C)n1cc(S(=O)(=O)NC(=O)Nc2c(-c3ccnc(C#N)c3)ccc3c2CCC3)ncc1=O ; CC(C)n1cc(S(=O)(=O)NC(=O)Nc2c(-c3ccnc(C#N)c3)ccc3c2CCC3)ncc1=O]

N-((5-(2-Cyanopyridin-4-yl)-2,3-dihydro-1H-inden-4-yl)carbamoyl)-4-isopropyl-5-oxo-4,5-dihydropyrazine-2-sulfonamide (0.015 g, 0.031 mmol) was treated with 0.1 M NaOH solution (310 μL) and the resultant solution was freeze-dried to afford the title compound (16 mg, quant. yield) as a yellow solid.

¹H NMR (DMSO-d6) δ 8.56 (d, J=5.1 Hz, 1H), 7.89 (t, J=1.6 Hz, 2H), 7.84 (d, J=1.1 Hz, 1H), 7.67-7.56 (m, 2H), 7.13-7.09 (m, 2H), 4.85 (sept, J=6.8 Hz, 1H), 2.90 (t, J=7.5 Hz, 2H), 2.77 (t, J=7.3 Hz, 2H), 1.98 (p, J=7.5 Hz, 2H), 1.28 (d, J=6.8 Hz, 6H).

LCMS: m/z 479.3 (M+H)⁺ (ES⁺); 477.1 (M−H)⁻ (ES⁻).

### Example 73: 4-Isopropyl-N-((5-(2-methoxypyridin-4-yl)-2,3-dihydro-1H-inden-4-yl)carbamoyl)-5-oxo-4,5-dihydropyrazine-2-sulfonamide, partial ammonium salt

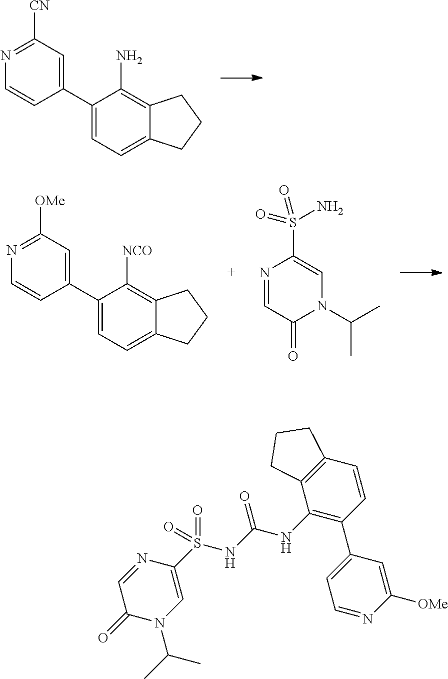

Prepared according to the general procedure of 1-isopropyl-N-((5-(2-methoxypyridin-4-yl)-2,3-dihydro-1H-inden-4-yl)carbamoyl)-6-oxo-1,6-dihydropyridine-3-sulfonamide (Example 70) from 4-isopropyl-5-oxo-4,5-dihydropyrazine-2-sulfonamide (Intermediate P13) (26 mg, 0.12 mmol) and 5-(2-methoxypyridin-4-yl)-2,3-dihydro-1H-inden-4-amine (Intermediate A35) (50 mg, 0.21 mmol) to afford the title compound (13.2 mg, 23%).

¹H NMR (DMSO-d6) δ 8.09 (s, 1H), 8.05 (d, J=5.3 Hz, 1H), 7.98 (s, 1H), 7.71 (s, 1H), 7.16 (d, J=7.7 Hz, 1H), 7.07 (d, J=7.6 Hz, 1H), 6.86 (d, J=5.3 Hz, 1H), 6.65 (s, 1H), 4.86 (sept, J=7.2, 6.7 Hz, 1H), 3.86 (s, 3H), 2.90 (t, J=7.4 Hz, 2H), 2.69 (t, J=7.5 Hz, 2H), 1.98 (p, J=7.4 Hz, 2H), 1.30 (d, J=6.7 Hz, 6H).

LCMS: m/z 484.3 (M+H)⁺ (ES⁺).

### Example 74: N-((2-(2-Cyanopyridin-4-yl)-4-fluoro-6-isopropylphenyl) carbamoyl)-1-isopropylazetidine-3-sulfonamide

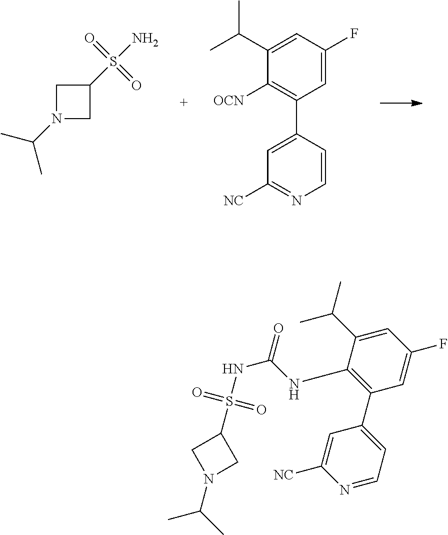

To a solution of 1-isopropylazetidine-3-sulfonamide (Intermediate P14) (70 mg, 392.70 μmol, 1 eq) in THF (2 mL) was added t-BuONa (37 mg, 392.70 μmol, 1 eq). The mixture was stirred at 25° C. for 30 minutes. Then 4-(5-fluoro-2-isocyanato-3-isopropylphenyl)picolinonitrile (Intermediate A37) (110 mg, 392.70 μmol, 1 eq) was added. The reaction mixture was stirred at 70° C. for 30 minutes. Then the reaction mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 12%-42%, 11.5 min) to give the title compound (80.02 mg, 43% yield, 96% purity on LCMS) as a white solid.

¹H NMR (DMSO-d₆) δ 8.75 (d, 1H), 8.06 (s, 1H), 7.77-7.66 (m, 2H), 7.21 (dd, 1H), 7.12 (dd, 1H), 3.78-3.49 (m, 4H), 3.26-3.22 (d, 2H), 2.83-2.79 (m, 1H), 1.15 (d, 6H) and 0.95 (d, 6H). One exchangeable proton not observed.

LCMS: m/z 460.2 (M+H)⁺ (ES⁺).

### Example 75: N-((4-Fluoro-2-isopropyl-6-(2-methoxypyridin-4-yl)phenyl) carbamoyl)-1-isopropylazetidine-3-sulfonamide

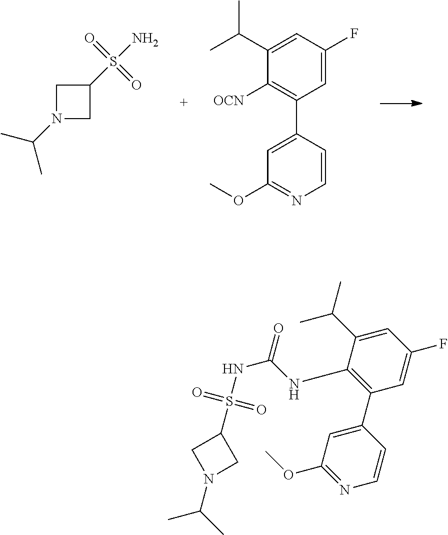

To a solution of 1-isopropylazetidine-3-sulfonamide (Intermediate P14) (70 mg, 392.70 mmol, 1 eq) in THF (2 mL) was added t-BuONa (38 mg, 392.70 μmol, 1 eq). The mixture was stirred at 25° C. for 30 minutes. Then 4-(5-fluoro-2-isocyanato-3-isopropylphenyl)-2-methoxypyridine (Intermediate A38) (112 mg, 392.70 μmol, 1 eq) was added. The mixture was stirred at 70° C. for 30 minutes. Then the reaction mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 12%-42%, 11.5 min) to give the title compound (87.88 mg, 48% yield, 99% purity on LCMS) as a white solid.

¹H NMR (DMSO-d₆) δ 8.11 (d, 1H), 7.17 (br s, 1H), 7.11 (d, 1H), 7.01 (s, 1H), 6.93 (d, 1H), 6.85 (s, 1H), 3.86 (s, 3H), 3.81-3.77 (m, 1H), 3.26-3.22 (m, 1H), 3.18-3.15 (m, 2H), 3.03-3.00 (m, 2H), 2.22-1.98 (m, 1H), 1.16-1.12 (m, 6H) and 0.80 (d, 6H). One exchangeable proton not observed.

LCMS: m/z 465.2 (M+H)⁺ (ES⁺).

### Example 76: 1-Isopropyl-N-((5-(2-methoxypyridin-4-yl)-2,3-dihydro-1H-inden-4-yl)carbamoyl)azetidine-3-sulfonamide

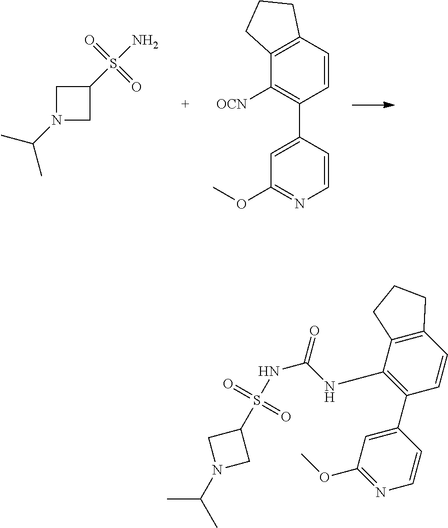

To a solution of 1-isopropylazetidine-3-sulfonamide (Intermediate P14) (70 mg, 392.70 μmol, 1 eq) in THF (2 mL) was added t-BuONa (38 mg, 392.70 μmol, 1 eq). The mixture was stirred at 25° C. for 30 minutes. Then 4-(4-isocyanato-2,3-dihydro-1H-inden-5-yl)-2-methoxypyridine (Intermediate A39) (104 mg, 392.70 μmol, 1 eq) was added. The mixture was stirred at 70° C. for 30 minutes. Then the reaction mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 8%-38%, 11.5 min) to give the title compound (56.2 mg, 32% yield, 100% purity on LCMS) as a white solid.

¹H NMR (DMSO-d₆) δ 8.13 (d, 1H), 7.49 (br s, 1H), 7.12 (d, 1H), 7.07 (d, 1H), 6.98 (d, 1H), 6.79 (s, 1H), 4.00-3.94 (m, 1H), 3.87 (s, 3H), 3.70-3.64 (m, 2H), 3.58-3.54 (m, 2H), 2.91 (t, 2H), 2.83 (t, 2H), 2.76-2.73 (m, 1H), 2.04-1-97 (m, 2H) and 0.94 (d, 6H). One exchangeable proton not observed.

LCMS: m/z 445.2 (M+H)⁺ (ES⁺).

### Example 77: N-((7-Fluoro-5-(pyridin-4-yl)-2,3-dihydro-1H-inden-4-yl) carbamoyl)-1-isopropylazetidine-3-sulfonamide

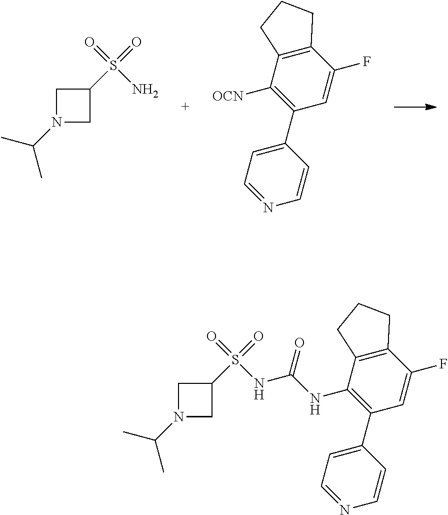

A mixture of 1-isopropylazetidine-3-sulfonamide (Intermediate P14) (50 mg, 280.50 μmol, 1 eq) and t-BuONa (27 mg, 280.50 μmol, 1 eq) in THF (2 mL) was stirred at 25° C. for 10 minutes. 4-(7-Fluoro-4-isocyanato-2,3-dihydro-1H-inden-5-yl)pyridine (Intermediate A40) (71 mg, 280.50 μmol, 1 eq) was added and the resulting mixture was stirred at 70° C. for 30 minutes. Then the reaction mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 12%-42%, 10 min) to give the title compound (7.96 mg, 7% yield, 100% purity on LCMS) as a white solid.

¹H NMR (DMSO-d₆) δ 8.55 (d, 2H), 7.4-7.38 (m, 3H), 6.95 (d, 1H), 3.94-3.88 (m, 1H), 3.70-3.67 (m, 2H), 3.61-3.58 (m, 2H), 2.95 (t, 2H), 2.86 (t, 2H), 2.82-2.75 (m, 1H), 2.10-2.02 (m, 2H) and 0.96 (d, 6H). One exchangeable proton not observed.

LCMS: m/z 433.2 (M+H)⁺ (ES^{+).}

### Example 78: N-((2-(2-Cyanopyridin-4-yl)-4-fluoro-6-isopropylphenyl) carbamoyl)-1-cyclobutylazetidine-3-sulfonamide

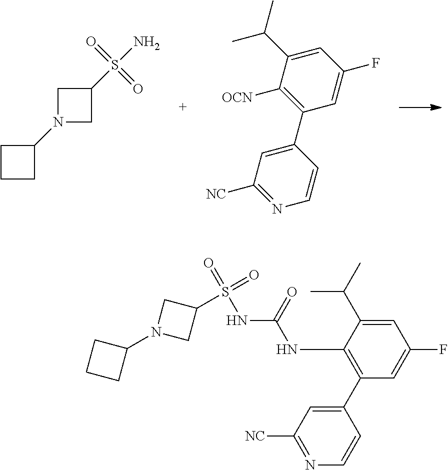

A solution of 1-cyclobutylazetidine-3-sulfonamide (Intermediate P15) (30 mg, 157.68 μmol, 1 eq) and t-BuONa (15 mg, 157.68 μmol, 1 eq) in THF (1 mL) was stirred at 25° C. for 10 minutes. 4-(5-Fluoro-2-isocyanato-3-isopropylphenyl)picolinonitrile (Intermediate A37) (44 mg, 157.68 μmol, 1 eq) was added and the resulting mixture was stirred at 25° C. for 10 minutes. Then the reaction mixture was concentrated in vacua. The residue was purified by prep-HPLC (Column: Waters Xbridge C18, 150 mm*25 mm*5% μm; mobile phase: [A: water (0.05% ammonium hydroxide v/v); B: MeCN]; B %: 5%-35%, 10 min) to give the title compound (6.35 mg, 8% yield, 97% purity on LCMS) as a white solid.

¹H NMR (DMSO-d₆) δ 8.75 (d, 1H), 8.05 (s, 1H), 7.77-7.75 (m, 1H), 7.67-7.65 (m, 1H), 7.23-7.18 (m, 1H), 7.12 (d, 1H), 3.95-3.68 (m, 2H), 3.67-3.56 (m, 2H), 3.55-3.42 (m, 2H), 3.25-3.21 (m, 1H), 1.99-1.97 (m, 2H), 1.86-1.84 (m, 2H), 1.71-1.62 (m, 2H) and 1.16 (d, 6H). One exchangeable proton not observed.

LCMS: m/z 472.2 (M+H)⁺ (ES⁺).

### Example 79: 1-Cyclobutyl-N-((4-fluoro-2-isopropyl-6-(2-methoxypyridin-4-yl)phenyl)carbamoyl)azetidine-3-sulfonamide

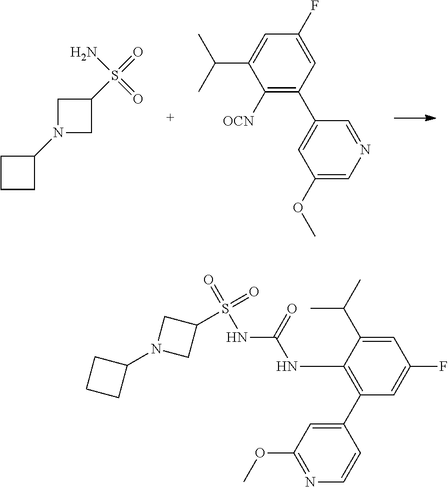

To a solution of 1-cyclobutylazetidine-3-sulfonamide (Intermediate P15) (25 mg, 131.40 μmol, 1 eq) in THF (1 mL) was added t-BuONa (13 mg, 131.40 μmol, 1 eq). The reaction mixture was stirred at 20° C. for 10 minutes. Then 4-(5-fluoro-2-isocyanato-3-isopropylphenyl)-2-methoxypyridine (Intermediate A38) (38 mg, 131.40 μmol, 1 eq) was added and the resulting mixture was stirred at 20° C. for 20 minutes. Then the reaction mixture was concentrated in vacuo. The residue was purified by prep-HPLC (column: Phenomenex Gemini C18, 150 mm*25 mm*10 μm; mobile phase: [A: water (10 mM NH₄HCO₃); B: MeCN]; B %: 15%-45%, 10 min) to give the title compound (41.16 mg, 66% yield, 100% purity on LCMS) as a white solid.

¹H NMR (DMSO-d₆) δ 8.16 (d, 1H), 7.61 (br s, 1H), 7.16 (d, 1H), 7.03-6.96 (m, 2H), 6.83 (s, 1H), 4.02-3.92 (m, 1H), 3.88 (s, 3H), 3.75-3.48 (m, 4H), 3.22-3.02 (m, 2H), 2.15-1.95 (m, 2H), 1.94-1.76 (m, 2H), 1.74-1.56 (m, 2H) and 1.14 (d, 6H). One exchangeable proton not observed.

LCMS: m/z 477.2 (M+H)⁺ (ES^{+).}

### Example 80: 1-Cyclobutyl-N-((5-(2-methoxypyridin-4-yl)-2,3-dihydro-1H-inden-4-yl)carbamoyl)azetidine-3-sulfonamide

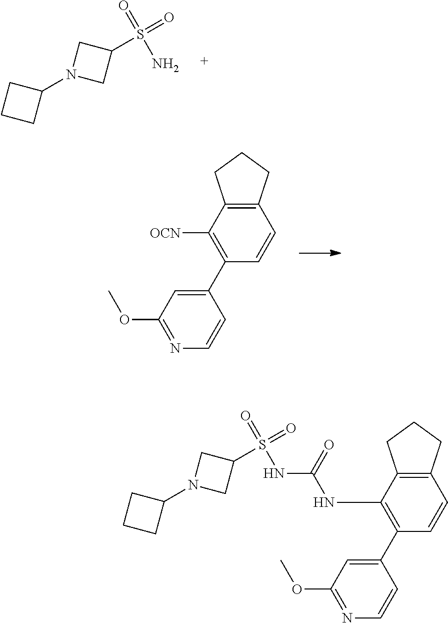

A mixture of 1-cyclobutylazetidine-3-sulfonamide (Intermediate P15) (40 mg, 210.24 μmol, 1 eq) and t-BuONa (20 mg, 210.24 μmol, 1 eq) in THF (2 mL) was stirred at 25° C. for 10 minutes. Then 4-(4-isocyanato-2,3-dihydro-1H-inden-5-yl)-2-methoxypyridine (Intermediate A39) (56 mg, 210.24 μmol, 1 eq) was added and the resulting mixture was stirred at 70° C. for 30 minutes. Then the reaction mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 10%-40%, 10 min) to give the title compound (20.06 mg, 21% yield, 100% purity on LCMS) as a white solid.

¹H NMR (DMSO-d₆) δ 8.13 (d, 1H), 7.40 (br s, 1H), 7.12 (d, 1H), 7.06 (d, 1H), 6.96 (d, 1H), 6.77 (s, 1H), 4.06-3.98 (m, 1H), 3.87 (s, 3H), 3.49-3.44 (m, 3H), 3.38-3.35 (m, 2H), 2.91 (t, 2H), 2.82 (t, 2H), 2.03-1.99 (m, 2H), 1.98-1.94 (m, 2H), 1.85-1.81 (m, 2H) and 1.71-1.62 (m, 2H). One exchangeable proton not observed.

LCMS: m/z 457.3 (M+H)⁺ (ES^{+).}

### Example 81: 1-Cyclobutyl-N-((7-fluoro-5-(pyridin-4-yl)-2,3-dihydro-1H-inden-4-yl)carbamoyl)azetidine-3-sulfonamide

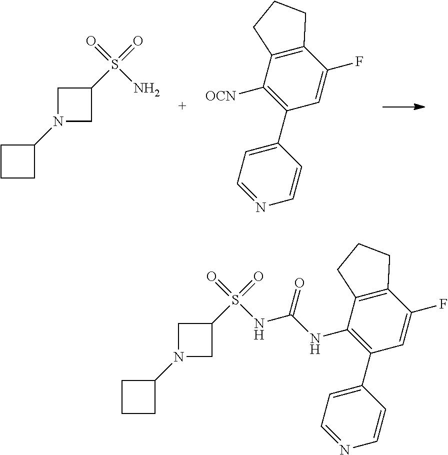

A mixture of 1-cyclobutylazetidine-3-sulfonamide (Intermediate P15) (37 mg, 194.47 μmol, 1 eq) and t-BuONa (19 mg, 194.47 μmol, 1 eq) in THF (2 mL) was stirred at 25° C. for 10 minutes. Then 4-(7-fluoro-4-isocyanato-2,3-dihydro-1H-inden-5-yl)pyridine (Intermediate A40) (49 mg, 194.47 μmol, 1 eq) was added and the resulting mixture was stirred at 25° C. for 10 minutes. Then the reaction mixture was concentrated in vacua. The residue was purified by prep-HPLC (column: Xtimate C18, 250 mm*50 mm*10 μm; mobile phase: [A: water (0.05% ammonium hydroxide v/v); B: MeCN]; B %: 0%-30%, 10 min) to give the title compound (18.09 mg, 20% yield, 97% purity on LCMS) as a yellow solid.

¹H NMR (DMSO-d₆) δ 8.57 (d, 2H), 7.57 (br s, 1H), 7.39 (d, 2H), 6.97 (d, 1H), 4.02-3.95 (m, 1H), 3.70-3.66 (m, 3H), 3.57-3.54 (m, 1H), 3.37-3.27 (m, 1H), 2.96 (t, 2H), 2.86 (t, 2H), 2.11-2.00 (m, 4H), 1.92-1.87 (m, 2H) and 1.72-1.65 (m, 2H). One exchangeable proton not observed.

LCMS: m/z 445.2 (M+H)⁺ (ES⁺).

### Example 82: N-((2-(2-Cyanopyridin-4-yl)-4-fluoro-6-isopropylphenyl) carbamoyl)-1-ethylazetidine-3-sulfonamide

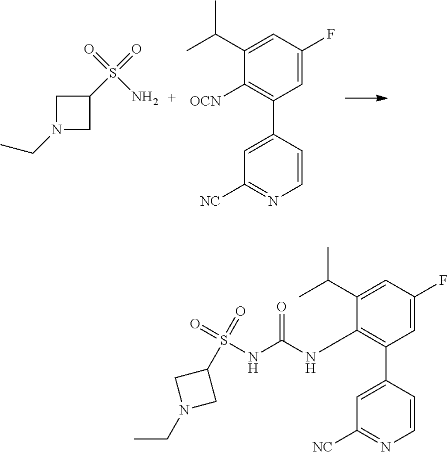

To a solution of 1-ethylazetidine-3-sulfonamide (Intermediate P16) (40 mg, 243.57 μmol, 1 eq) in THF (1 mL) was added t-BuONa (23 mg, 243.57 μmol, 1 eq). The mixture was stirred at 25° C. for 10 minutes. Then 4-(5-fluoro-2-isocyanato-3-isopropylphenyl)-picolinonitrile (Intermediate A37) (68 mg, 243.57 μmol, 1 eq) was added and the mixture was stirred at 70° C. for 10 minutes. Then the reaction mixture was concentrated in vacuo. The residue was purified by prep-HPLC (column: Waters Xbridge C18, 150 mm*50 mm*10 μm; mobile phase: [A: water (0.05% ammonium hydroxide v/v); B: MeCN]; B %: 8%-38%, 11.5 min) to give the title compound (48.97 mg, 45% yield, 100% purity on LCMS) as a white solid.

¹H NMR (DMSO-d₆) δ 8.75 (d, 1H), 8.05 (s, 1H), 7.76 (s, 1H), 7.66 (s, 1H), 7.22-7.18 (m, 1H), 7.12-7.09 (m, 1H), 3.83-3.76 (m, 5H), 3.24-3.20 (m, 1H), 2.93-2.88 (m, 2H), 1.16 (d, 6H) and 0.99 (t, 3H). One exchangeable proton not observed.

LCMS: m/z 446.2 (M+H)⁺ (ES⁺).

### Example 83: 1-Ethyl-N-((4-fluoro-2-isopropyl-6-(2-methoxypyridin-4-yl) phenyl)carbamoyl)azetidine-3-sulfonamide

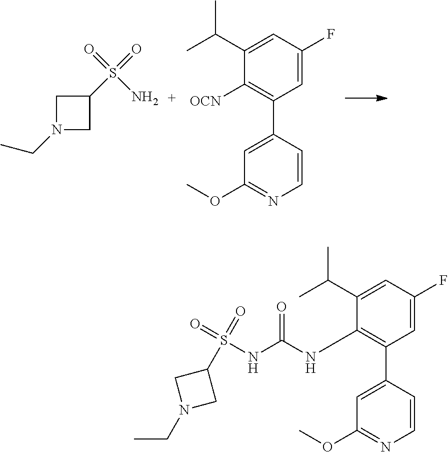

To a solution of 1-ethylazetidine-3-sulfonamide (Intermediate P16) (40 mg, 243.57 μmol, 1 eq) in THF (1 mL) was added t-BuONa (23 mg, 243.57 μmol, 1 eq). The mixture was stirred at 25° C. for 10 minutes. Then 4-(5-fluoro-2-isocyanato-3-isopropylphenyl)-2-methoxypyridine (Intermediate A38) (69 mg, 243.57 μmol, 1 eq) was added and the mixture was stirred at 75° C. for another 10 minutes. Then the reaction mixture was concentrated in vacuo. The residue was purified by prep-HPLC (column: Waters Xbridge C18, 150 mm*50 mm*10 μm; mobile phase: [A: water (0.05% ammonium hydroxide v/v); B: MeCN]; B %: 8%-38%, 11.5 min) to give the title compound (46.05 mg, 42% yield, 100% purity on LCMS) as a white solid.

¹H NMR (DMSO-d₆) δ 8.15 (d, 1H), 7.48 (s, 1H), 7.17-7.12 (m, 1H), 7.03-6.94 (m, 2H), 6.84 (s, 1H), 3.99-3.77 (m, 8H), 3.24-3.20 (m, 1H), 2.95-2.92 (m, 2H), 1.15 (d, 6H) and 1.00 (t, 3H). One exchangeable proton not observed.

LCMS: m/z 451.2 (M+H)⁺ (ES⁺).

### Example 84: 1-Ethyl-N-((5-(2-methoxypyridin-4-yl)-2,3-dihydro-1H-inden-4-yl)carbamoyl)azetidine-3-sulfonamide

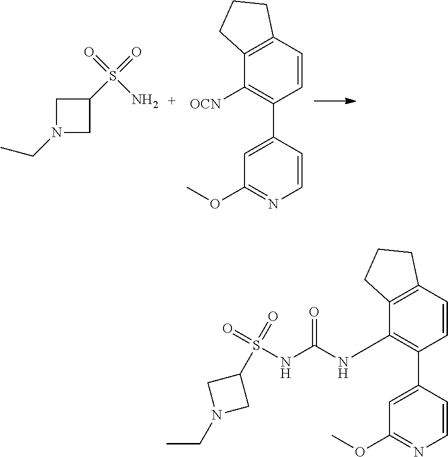

To a solution of 1-ethylazetidine-3-sulfonamide (Intermediate P16) (40 mg, 243.57 μmol, 1 eq) in THF (1 mL) was added t-BuONa (23 mg, 243.57 μmol, 1 eq). The mixture was stirred at 25° C. for 10 minutes. Then 4-(4-isocyanato-2,3-dihydro-1H-inden-5-yl)-2-methoxypyridine (Intermediate A39) (64 mg, 243.57 μmol, 1 eq) was added and the mixture was stirred at 70° C. for 10 minutes. Then the reaction mixture was concentrated in vacuo. The residue was purified by prep-HPLC (column: Waters Xbridge C18, 150 mm*50 mm*10 μm; mobile phase: [A: water (0.05% ammonium hydroxide v/v); B: MeCN]; B %: 8%-38%, 11.5 min) to give the title compound (52.99 mg, 51% yield, 100% purity on LCMS) as a white solid.

¹H NMR (DMSO-d₆) δ 8.13 (d, 1H), 7.43 (br s, 1H), 7.12 (d, 1H), 7.06 (d, 1H), 6.97 (dd, 1H), 6.79 (s, 1H), 4.08-4.00 (m, 1H), 3.88 (s, 3H), 3.85-3.80 (m, 2H), 3.77-3.72 (m, 2H), 2.91 (t, 2H), 2.87-2.80 (m, 4H), 2.04-1.96 (m, 2H) and 0.98 (t, 3H). One exchangeable proton not observed.

LCMS: m/z 431.2 (M+H)⁺ (ES⁺).

### Example 85: 1-Ethyl-N-((7-fluoro-5(pyridin-4-yl)-2,3-dihydro-1H-inden-4-yl)carbamoyl)azetidine-3-sulfonamide

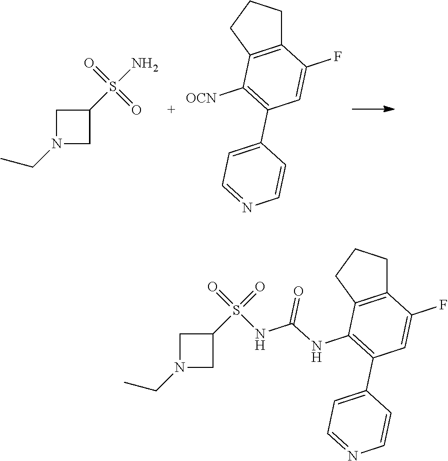

A solution of 1-ethylazetidine-3-sulfonamide (Intermediate P16) (50 mg, 304.46 μmol, 1 eq) and t-BuONa (29 mg, 304.46 μmol, 1 eq) in THF (1 mL) was stirred at 25° C. for 10 minutes. Then a solution of 4-(7-fluoro-4-isocyanato-2,3-dihydro-1H-inden-5-yl) pyridine (Intermediate A40) (77 mg, 304.46 μmol, 1 eq) in THF (2 mL) was added and the reaction mixture was stirred at 25° C. for 10 minutes. Then the reaction mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 10 min) to give the title compound (9.59 mg, 8% yield, 100% purity on LCMS) as a white solid.

¹H NMR (DMSO-d₆) δ 8.57 (d, 2H), 7.43 (br s, 1H), 7.40 (d, 2H), 6.96 (d, 1H), 4.01-3.88 (m, 5H), 2.98-2.93 (m, 4H), 2.86 (t, 2H), 2.11-2.03 (m, 2H) and 1.01 (t, 3H). One exchangeable proton not observed.

LCMS: m/z 419.2 (M+H)⁺ (ES⁺).

### Example 86: N-((2-(2-Cyanopyridin-4-yl)-4-fluoro-6-isopropylphenyl) carbamoyl)-1-(pyridin-3-ylmethyl)azetidine-3-sulfonamide

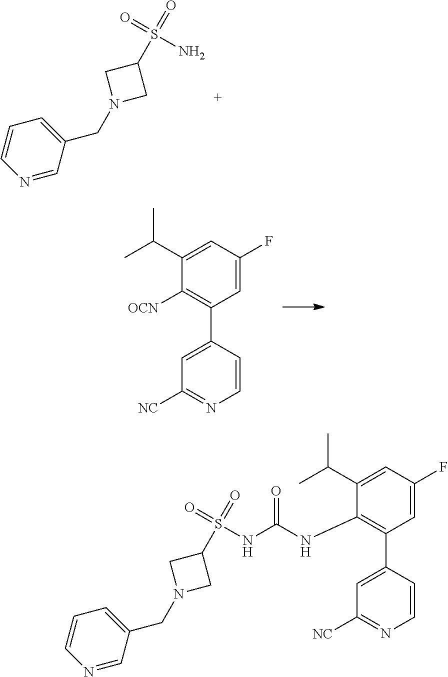

A solution of 1-(pyridin-3-ylmethyl)azetidine-3-sulfonamide (Intermediate P17) (50 mg, 219.99 μmol, 1 eq), 4-(5-fluoro-2-isocyanato-3-isopropylphenyl)picolinonitrile (Intermediate A37) (68 mg, 241.99 μmol, 1.1 eq) and t-BuONa (25 mg, 263.99 μmol, 1.2 eq) in THF (1.5 mL) was stirred at 16° C. for 0.5 hour. Then the reaction mixture was concentrated in vacuo. The residue was purified by prep-HPLC (column: Phenomenex Gemini C18, 150 mm*25 mm*10 μm; mobile phase: [A: water (0.05% NH₄HCO₃ v/v); B: MeCN]; B %: 15%-45%, 12 min) to give the title compound (10 mg, 9%) as a white solid.

¹H NMR (DMSO-d₆) δ 8.74 (d, 1H), 8.50-8.47 (m, 2H), 8.05 (s, 1H), 8.00 (br s, 1H), 7.73 (d, 1H), 7.68 (d, 1H), 7.39-7.35 (m, 1H), 7.29-7.25 (m, 1H), 7.16 (d, 1H), 4.03-3.97 (m, 1H), 3.73-3.68 (m, 2H), 3.45-3.38 (m, 4H), 3.19-3.15 (m, 1H) and 1.14 (d, 6H). One exchangeable proton not observed.

LCMS: m/z 509.3 (M+H)⁺ (ES^{+).}

### Example 87: N-((5-(2-Methoxypyridin-4-yl)-2,3-dihydro-1H-inden-4-yl) carbamoyl)-1-(pyridin-3-ylmethyl)azetidine-3-sulfonamide

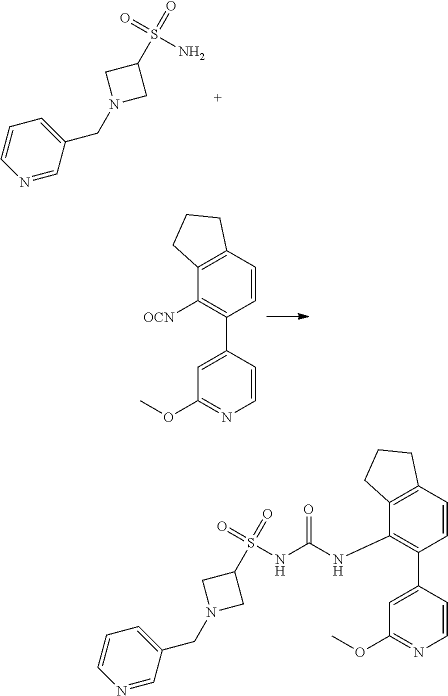

A solution of 1-(pyridin-3-ylmethyl)azetidine-3-sulfonamide (Intermediate P17) (50 mg, 219.99 μmol, 1 eq), 4-(4-isocyanato-2,3-dihydro-1H-inden-5-yl)-2-methoxypyridine (Intermediate A39) (64 mg, 241.99 μmol, 1.1 eq) and t-BuONa (25 mg, 263.99 μmol, 1.2 eq) in THF (1.5 mL) was stirred at 16° C. for 0.5 hour. Then the reaction mixture was concentrated in vacuo. The residue was purified by prep-HPLC (column: Phenomenex Gemini C18, 150 mm*25 mm*10 μm; mobile phase: [A: water (0.05% NH₄HCO₃ v/v); B: MeCN]; B %: 15%-45%, 12 min) to give the title compound (37 mg, 34%) as a white solid.

¹H NMR (DMSO-d₆) δ 8.49-8.45 (m, 2H), 8.12 (d, 1H), 7.79 (br s, 1H), 7.67 (d, 1H), 7.38-7.33 (m, 1H), 7.18 (d, 1H), 7.09 (d, 1H), 6.92 (d, 1H), 6.73 (s, 1H), 4.19-4.15 (m, 1H), 3.80 (s, 3H), 3.66 (s, 2H), 3.50-3.43 (m, 2H), 3.38-3.34 (m, 2H), 2.91 (t, 2H), 2.78 (t, 2H) and 2.04-1.98 (m, 2H). One exchangeable proton not observed.

LCMS: m/z 494.2 (M+H)⁺ (ES⁺).

### Example 88: N-((7-Fluoro-5-(pyridin-4-yl)-2,3-dihydro-1H-inden-4-yl) carbamoyl)-1-(pyridin-3-ylmethyl)azetidine-3-sulfonamide

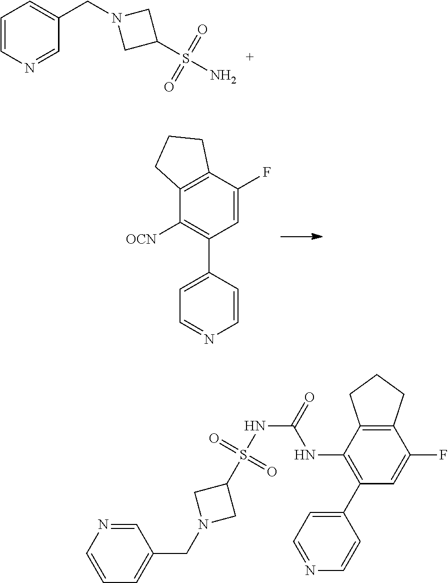

To a solution of 1-(pyridin-3-ylmethyl)azetidine-3-sulfonamide (Intermediate P17) (54 mg, 235.98 μmol, 1 eq) in THF (5 mL) was added t-BuONa (27 mg, 283.18 μmol, 1.2 eq) and a solution of 4-(7-fluoro-4-isocyanato-2,3-dihydro-1H-inden-5-yl)pyridine (Intermediate A40) (60 mg, 235.98 μmol, 1 eq) in THF (5 mL) and DCM (5 mL). The reaction mixture was stirred at 16° C. for 0.5 hour. Then the reaction mixture was concentrated in vacuo. The residue was purified by prep-HPLC (column: Phenomenex Gemini C18, 150 mm*25 mm*10 μm; mobile phase: [A: water (0.05% NH₄HCO₃ v/v); B: MeCN]; B %: 5%-50%, 10 min) to give the title compound (35.53 mg, 31% yield, 99.4% purity on LCMS) as a white solid.

¹H NMR (DMSO-do) δ 8.56-8.54 (m, 2H), 8.49-8.47 (m, 2H), 7.76 (br s, 1H), 7.68 (d, 1H), 7.36 (dd, 3H), 7.00 (d, 1H), 4.17-4.12 (m, 1H), 3.68 (s, 2H), 3.47 (t, 2H), 3.40 (t, 2H), 2.96 (t, 2H), 2.84 (t, 2H) and 2.11-2.03 (m, 2H). One exchangeable proton not observed.

LCMS: m/z 482.2 (M+H)⁺ (ES⁺).

### Example 89: N-((2-(2-cyanopyridin-4-yl)-4-fluoro-6-isopropylphenyl) carbamoyl)-1-isopropyl-6-oxo-1,6-dihydropyridine-3-sulfonamide

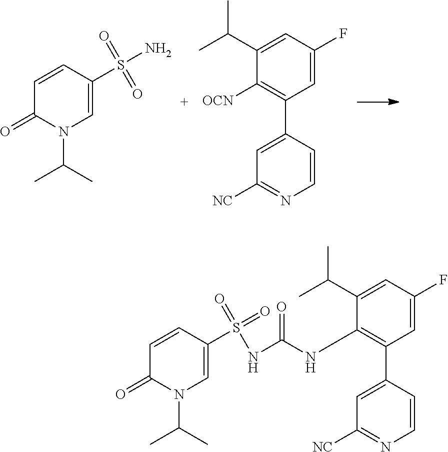

A solution of 1-isopropyl-6-oxo-1,6-dihydropyridine-3-sulfonamide (Intermediate P12) (60 mg, 225.09 μmol, 1 eq), 4-(5-fluoro-2-isocyanato-3-isopropylphenyl)-picolinonitrile (Intermediate A37) (70 mg, 247.60 μmol, 1.1 eq) and t-BuONa (26 mg, 270.11 μmol, 1.2 eq) in THF (1.5 mL) was stirred at 16° C. for 0.5 hour. Then the reaction mixture was concentrated in vacuo. The residue was purified by prep-HPLC (column: Phenomenex Gemini C18, 150 mm*25 mm*10 μm; mobile phase: [A: water (0.05% NH₄HCO₃ v/v); B: MeCN]; B %: 15%-45%, 12 min) to give the title compound (30 mg, 26%) as a white solid.

¹H NMR (DMSO-d₆) δ 8.57 (d, 1H), 7.99-7.92 (m, 3H), 7.64-7.62 (m, 1H), 7.47-7.45 (m, 1H), 7.25-7.22 (m, 1H), 7.14-7.11 (m, 1H), 6.36 (d, 1H), 4.99-4.91 (m, 1H), 3.10-3.05 (m, 1H), 1.25 (d, 6H) and 1.09 (d, 6H). One exchangeable proton not observed.

LCMS: m/z 498.3 (M+H)⁺ (ES⁺).

### Example 90: N-((4-Fluoro-2-isopropyl-6-(2-methoxypyridin-4-yl)phenyl) carbamoyl)-1-isopropyl-6-oxo-1,6-dihydropyridine-3-sulfonamide

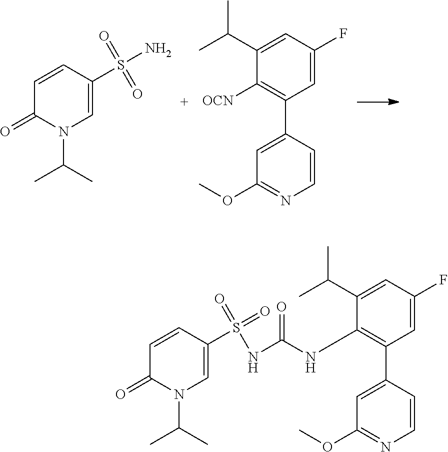

A solution of 1-isopropyl-6-oxo-1,6-dihydropyridine-3-sulfonamide (Intermediate P12) (60 mg, 225.09 μmol, 1 eq), 4-(5-fluoro-2-isocyanato-3-isopropylphenyl)-2-methoxypyridine (Intermediate A38) (71 mg, 247.60 μmol, 1.1 eq) and t-BuONa (26 mg, 270.11 μmol, 1.2 eq) in THF (1.5 mL) was stirred at 16° C. for 0.5 hour. Then the reaction mixture was concentrated in vacuo. The residue was purified by prep-HPLC (column: Xtimate C18, 250 mm*50 mm*10 μm; mobile phase: [A: water (0.05% ammonium hydroxide v/v); B: MeCN]; B %: 2%-32%, 10 min) to give the title compound (61 mg, 54%) as a white solid.

¹H NMR (DMSO-d₆) δ 7.97 (d, 2H), 7.51 (d, 2H), 7.13 (dd, 1H), 6.96-6.89 (m, 2H), 6.73 (s, 1H), 6.35 (d, 1H), 5.00-4.95 (m, 1H), 3.83 (s, 3H), 3.09-3.04 (m, 1H), 1.25 (d, 6H) and 1.05 (d, 6H). One exchangeable proton not observed.

LCMS: m/z 503.2 (M+H)⁺ (ES⁺).

### Example 91: N-((7-Fluoro-5-(pyridin-4-yl)-2,3-dihydro-1H-inden-4-yl) carbamoyl)-1-isopropyl-6-oxo-1,6-dihydropyridine-3-sulfonamide

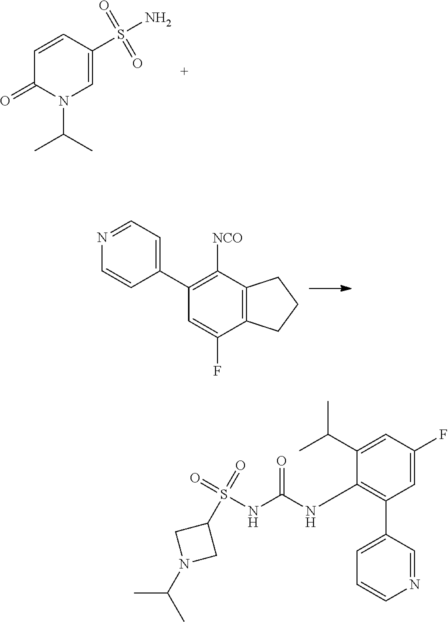

A solution of 1-isopropyl-6-oxo-1,6-dihydropyridine-3-sulfonamide (Intermediate P12) (50 mg, 187.58 μmol, 1 eq), 4-(7-fluoro-4-isocyanato-2,3-dihydro-1H-inden-5-yl)pyridine (Intermediate A40) (52 mg, 206.34 μmol, 1.1 eq) and t-BuONa (22 mg, 225.10 μmol, 1.2 eq) in THF (1.5 mL) was stirred at 16° C. for 0.5 hour. Then the reaction mixture was concentrated in vacuo. The residue was purified by prep-HPLC (column: Phenomenex Gemini C18, 150 mm*25 mm*10 μm; mobile phase: [A: water (0.05% NH₄HCO₃ v/v); B: MeCN]; B %:12%-42%, 12 min) to give the title compound (6 mg, 7% yield, 99.17% purity on LCMS) as a white solid.

¹H NMR (DMSO-d₆) δ 8.46 (d, 2H), 8.08 (s, 1H), 7.83 (br s, 1H), 7.58 (dd, 1H), 7.26 (d, 2H), 6.99 (d, 1H), 6.45 (d, 1H), 5.02-4.94 (m, 1H), 2.94 (t, 2H), 2.71 (t, 2H), 2.07-2.01 (m, 2H) and 1.28 (d, 6H). One exchangeable proton not observed.

LCMS: m/z 471.2 (M+H)⁺ (ES⁺).

### Example 92: N-((4-Fluoro-2-isopropyl-6-(pyridin-3-yl)phenyl)carbamoyl)-1-isopropylazetidine-3-sulfonamide

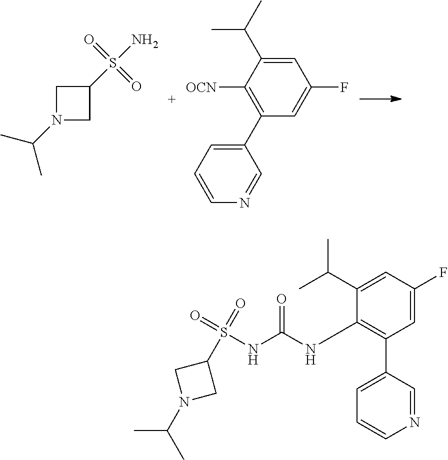

To a solution of 1-isopropylazetidine-3-sulfonamide (Intermediate P14) (200 mg, 1.12 mmol, 1 eq) in THF (5 mL) was added MeONa (60 mg, 1.12 mmol, 1 eq). The reaction mixture was stirred at 25° C. for 30 minutes. Then 3-(5-fluoro-2-isocyanato-3-isopropylphenyl)pyridine (Intermediate A41) (431 mg, 1.68 mmol, 1.5 eq) was added and the resulting mixture was stirred at 70° C. for 30 minutes. Then the reaction mixture was concentrated in vacuo. The residue was purified by reversed phase flash chromatography (column: Welch Ultimate XB_C18, 35 mm*235 mm*20/35 μm, mobile phase: [A: water (0.05% ammonium hydroxide); B: MeCN]; B %: 0%-40%, 10 min) to give the title compound (33 mg, 7% yield, 100% purity on LCMS) as a white solid.

¹H NMR (DMSO-d₆) δ 8.60-8.51 (m, 2H), 7.92-7.77 (m, 1H), 7.57 (s, 1H), 7.44-7.40 (m, 1H), 7.14 (d, 1H), 7.00 (d, 1H), 3.92-3.74 (m, 3H), 3.29-2.95 (m, 4H), 1.26-1.10 (m, 6H) and 1.02 (d, 6H). One exchangeable proton not observed.

LCMS: m/z 435.2 (M+H)⁺ (ES⁺).

### Example 93: N-((4-Fluoro-2-isopropyl-6-(pyridin-3-yl)phenyl)carbamoyl)-1-isopropylpiperidine-4-sulfonamide

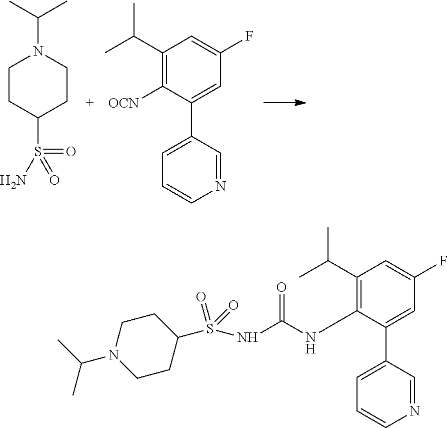

To a solution of 1-isopropylpiperidine-4-sulfonamide (Intermediate P18) (720 mg, 3.49 mmol, 1 eq) in THF (10 mL) was added NaOMe (226 mg, 4.19 mmol, 1.2 eq) and 3-(5-fluoro-2-isocyanato-3-isopropylphenyl)pyridine (Intermediate A41) (805 mg, 3.14 mmol, 0.9 eq). Then the reaction mixture was stirred at 70° C. for 20 minutes. The reaction mixture was concentrated in vacuo. The residue was purified by prep-HPLC (column: Phenomenex Gemini C18, 250 mm*50 mm*10 μm; mobile phase: [A: water (10 mM NH₄HCO₃); B: MeCN]; B %: 15%-45%, 10 min) to give the title compound (69.36 mg, 4% yield, 100% purity on LCMS) as a white solid.

¹H NMR (DMSO-d₆) δ 8.57 (s, 1H), 8.48 (d, 1H), 7.87-7.80 (m, 1H), 7.36-7.32 (m, 1H), 7.25 (s, 1H), 7.10 (d, 1H), 6.95 (d, 1H), 6.09 (s, 1H), 2.95-2.85 (m, 1H), 2.79-2.76 (m, 2H), 2.70-2.63 (m, 2H), 1.98-1.85 (m, 2H), 1.65-1.61 (m, 2H), 1.42-1.38 (m, 2H), 1.14 (d, 6H) and 0.94 (d, 6H). One exchangeable proton not observed.

LCMS: m/z 463.4 (M+H)⁺ (ES⁺).

### Example 94: N-((4-Fluoro-2-isopropyl-6-(2-methoxypyridin-4-yl)phenyl) carbamoyl)-1-(pyridin-3-ylmethyl)azetidine-3-sulfonamide

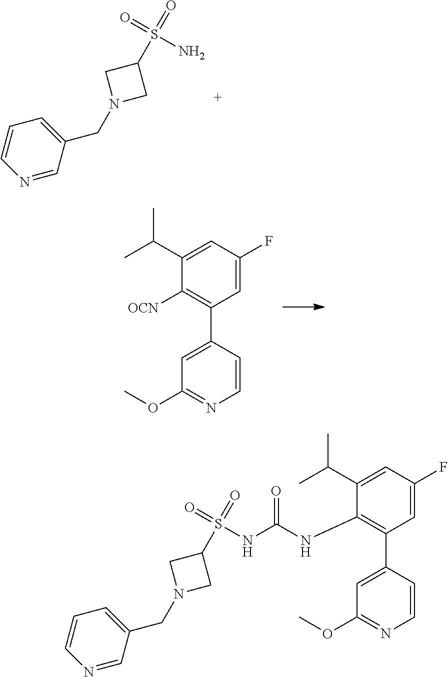

A solution of 1-(pyridin-3-ylmethyl)azetidine-3-sulfonamide (Intermediate P17) (50 mg, 219.99 μmol, 1 eq), 4-(5-fluoro-2-isocyanato-3-isopropylphenyl)-2-methoxypyridine (Intermediate A38) (69 mg, 241.99 μmol, 1.1 eq) and t-BuONa (25 mg, 263.99 μmol, 1.2 eq) in THF (1.5 mL) was stirred at 16° C. for 0.5 hour. Then the reaction mixture was concentrated in vacuo. The residue was purified by prep-HPLC (column: Waters Xbridge C18, 150 mm*50 mm*10 μm, mobile phase: [A: water (0.05% ammonium hydroxide v/v); B: MeCN]; B %: 8%-38%, 11.5 min) to give the title compound (44 mg, 38%) as a white solid.

¹H NMR (DMSO-d₆) δ 8.47 (s, 2H), 8.12 (d, 1H), 7.67 (d, 2H), 7.35 (dd, 1H), 7.19 (d, 1H), 7.01-6.95 (m, 2H), 6.80 (s, 1H), 4.04-3.98 (m, 1H), 3.78 (s, 3H), 3.64 (s, 2H), 3.43-3.36 (m, 4H), 3.16-3.12 (m, 1H) and 1.12 (d, 6H). One exchangeable proton not observed.

LCMS: m/z 514.3 (M+H)⁺ (ES⁺).

### Example 95: 1-Isopropyl-N-((5-(2-methoxypyridin-4-yl)-2,3-dihydro-1H-inden-4-yl)carbamoyl)-2-oxo-1,2-dihydropyrimidine-5-sulfonamide, sodium salt

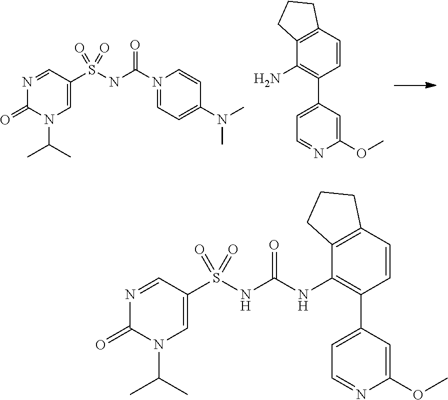

A suspension of 5-(2-methoxypyridin-4-yl)-2,3-dihydro-1H-inden-4-amine (0.033 g, 0.137 mmol) (Intermediate A35) and (4-(dimethylamino)pyridin-1-ium-1-carbonyl)((1-isopropyl-2-oxo-1,2-dihydropyrimidin-5-yl)sulfonyl)amide (Intermediate P19) (0.069 g, 0.123 mmol) in dry MeCN (2 mL) was stirred at 50° C. for 2 hours. Then the reaction mixture was concentrated in vacua and the crude product was purified by prep-HPLC (column: Waters Xbridge C18, 19 mm*15 mm*5 μm; mobile phase: [A: water (0.1% NH₄HCO₃); B: MeCN]; B %: 10%-40%) to afford 1-isopropyl-N-((5-(2-methoxypyridin-4-yl)-2,3-dihydro-1H-inden-4-yl)carbamoyl)-2-oxo-1,2-dihydropyrimidine-5-sulfonamide (0.031 g, 52%) as a flocculent white solid. The free acid (0.024 g, 0.050 mmol) was treated with 0.1 M NaOH (aq) (0.500 ml, 0.05 mmol) and the resultant solution was freeze-dried to afford the title compound (0.025 g, 99%) as a white solid.

¹H NMR (DMSO-d6) δ 8.65 (d, J=3.0 Hz, 1H), 8.35 (d, J=3.1 Hz, 1H), 7.98 (d, J=5.2 Hz, 1H), 7.24 (br s, 1H), 7.08 (d, J=7.7 Hz, 1H), 7.03 (d, J=7.6 Hz, 1H), 6.88 (dd, J=5.3, 1.4 Hz, 1H), 6.70 (t, J=1.0 Hz, 1H), 4.76 (sept, J=6.7 Hz, 1H), 3.82 (s, 3H), 2.88 (t, J=7.4 Hz, 2H), 2.70 (t, J=7.4 Hz, 2H), 1.94 (1), J=7.5 Hz, 2H), 1.30 (d, J=6.8 Hz, 6H).

LCMS: m/z 484.1 (M+H)⁺ (ES⁺); 482.1 (M−H)⁻ (ES⁻).

### Example 96: 1-Isopropyl-N-((5-(2-methoxypyridin-4-yl)-2,3-dihydro-1H-inden-4-yl)carbamoyl)-2-oxo-1,2-dihydropyridine-4-sulfonamide, sodium salt

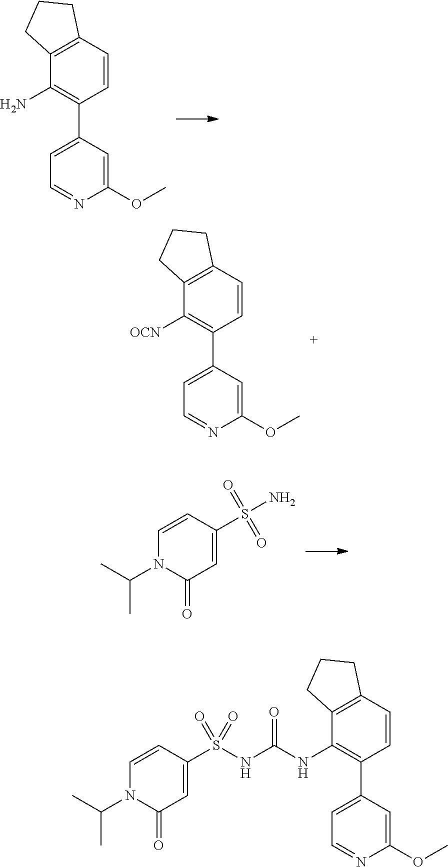

To a solution of 5-(2-methoxypyridin-4-yl)-2,3-dihydro-1H-inden-4-amine (Intermediate A35) (0.156 g, 0.65 mmol) in DCM (5 mL) and saturated aqueous NaHCO₃ (5 mL) was added a solution of bis(trichloromethyl) carbonate (0.079 g, 0.264 mmol) in toluene (1 mL) to the DCM layer without stirring. The reaction mixture was stirred for 1 hour, passed through a phase separator, dried (MgSO₄), filtered and concentrated in vacuo to afford crude isocyanate intermediate as an orange oil which was used without further purification. The crude isocyanate intermediate was dissolved in dry THF (11 mL).

A solution of 1-isopropyl-2-oxo-1,2-dihydropyridine-4-sulfonamide (Intermediate P20) (0.050 g, 0.224 mmol) in dry THF (3 mL) was treated with sodium tert-butoxide (2 M in THF) (0.120 ml, 0.24 mmol). The reaction mixture was stirred at room temperature for 1 hour, treated with a solution of the crude isocyanate intermediate in dry THF (4 mL) and then stirred at room temperature for 22 hours. The reaction mixture was concentrated in vacuo and the residue purified by reversed phase flash C18 chromatography (liquid load) (12 g cartridge, 5-50% MeCN/10 mM ammonium bicarbonate) to afford 1-isopropyl-N-((5-(2-methoxypyridin-4-yl)-2,3-dihydro-1H-inden-4-yl)carbamoyl)-2-oxo-1,2-dihydropyridine-4-sulfonamide (0.079 g, 70%) as a flocculent white solid. The free acid (0.071 g, 0.141 mmol) was treated with 0.1 M NaOH (aq) (1.410 ml, 0.141 mmol) and the mixture was freeze-dried to afford the title compound (0.073 g, 102%) as a white solid.

¹H NMR (DMSO-d6) δ 8.06 (dd, J=5.3, 0.7 Hz, 1H), 7.87 (dd, J=6.9, 2.1 Hz, 1H), 7.76 (dd, J=7.0, 2.1 Hz, 1H), 7.30 (br s, 1H), 7.06 (d, J=7.7 Hz, 1H), 7.03 (d, J=7.7 Hz, 1H), 6.94 (dd, J=5.3, 1.5 Hz, 1H), 6.76 (t, J=1.0 Hz, 1H), 6.30 (t, J=6.9 Hz, 1H), 5.14 (sept, J=6.8 Hz, 1H), 3.85 (s, 3H), 2.85 (t, J=7.4 Hz, 2H), 2.67 (t, J=7.4 Hz, 2H), 1.90 (p, J=7.5 Hz, 2H), 1.30 (d, J=6.8 Hz, 6H).

LCMS: m/z 483.1 (M+H)⁺ (ES⁺); 481.0 (M−H)⁻ (ES⁻).

### Example 97: 1-Ethyl-N-((7-fluoro-5-(2-methoxypyridin-4-yl)-2,3-dihydro-1H-inden-4-yl)carbamoyl)piperidine-4-sulfonamide, potassium salt

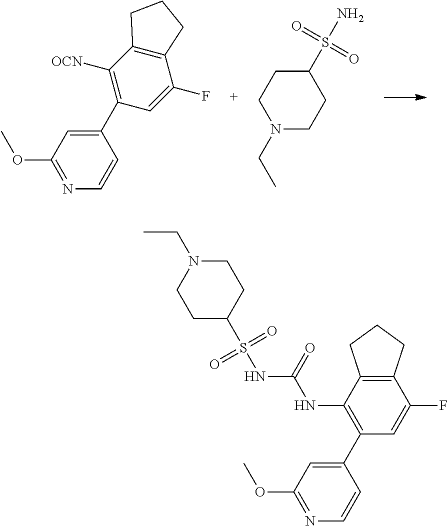

To a solution of 1-ethylpiperidine-4-sulfonamide (Intermediate P11; 90 mg, 0.37 mmol) in THF (5 mL) was added potassium tert-butoxide (49 mg, 0.44 mmol). The mixture was stirred at room temperature for 45 minutes. Then 4-(7-fluoro-4-isocyanato-2,3-dihydro-1H-inden-5-yl)-2-methoxypyridine (Intermediate A42; 90 mg, 0.32 mmol) was added and the mixture was stirred for 2 hours room temperature. The reaction mixture was concentrated in vacua and DMSO (0.5-1 mL) was added. The mixture (filtered over cotton wool when solids were present) was submitted for purification by reversed phase column chromatography (see “Experimental Methods”, preparative reversed phase HPLC method 4) to afford the title compound (18 mg, 10%) as a white solid.

¹H NMR (methanol-d₄) δ 8.10 (d, 1H), 7.03 (d, 1H), 6.87 (s, 1H), 6.84 (s, 1H), 3.92 (s, 3H), 3.23 (m, 2H), 3.07 (m, 1H), 3.00 (m, 4H), 2.68 (m, 2H), 2.32-2.08 (m, 4H), 2.03 (m, 2H), 1.86 (m, 2H), 1.18 (t, 3H).

LCMS: m/z 477 (M+H)⁺ (ES⁺); 475 (M−H)⁻ (ES⁻).

### Example 98: 1-Ethyl-N-((5(2-methoxypyridin-4-yl)-2,3-dihydro-1H-inden-4-yl)carbamoyl)piperidine-4-sulfonamide, potassium salt

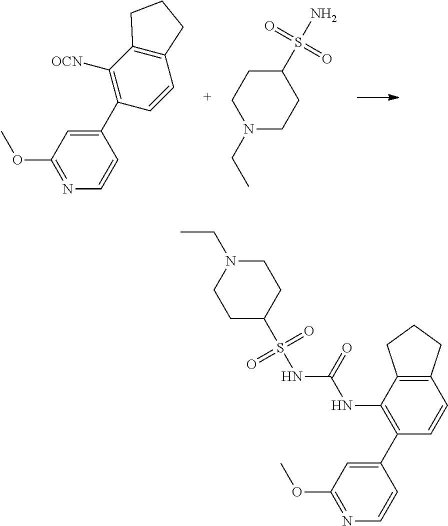

Prepared as described for 1-ethyl-N-((7-fluoro-5-(2-methoxypyridin-4-yl)-2,3-dihydro-1H-inden-4-yl)carbamoyl)piperidine-4-sulfonamide, potassium salt (Example 97) using 4-(4-isocyanato-2,3-dihydro-1H-inden-5-yl)-2-methoxypyridine (Intermediate A39) and 1-ethylpiperidine-4-sulfonamide (Intermediate Pu) to afford the title compound (54 mg, 30%) as a white solid.

¹H NMR (methanol-d₄) δ 8.08 (d, 1H), 7.25-7.08 (m, 2H), 7.03 (dd, 1H), 6.86 (s, 1H), 3.92 (s, 3H), 3.39-3.17 (m, 3H), 2.95 (m, 4H), 2.71 (q, 2H), 2.33 (t, 2H), 2.22-1.97 (m, 4H), 1.97-1.72 (m, 2H), 1.18 (t, 3H).

LCMS: m/z 459 (M+H)⁺ (ES⁺); 457 (M−H)⁻ (ES⁻).

### Example 99: N-((5-(2-Cyanopyridin-4-yl)-2,3-dihydro-1H-inden-4-yl) carbamoyl)-1-ethylpiperidine-4-sulfonamide, potassium salt

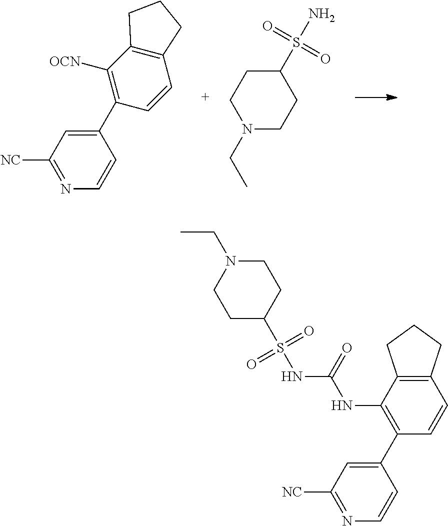

Prepared as described for 1-ethyl-N-((7-fluoro-5-(2-methoxypyridin-4-yl)-2,3-dihydro-1H-inden-4-yl)carbamoyl)piperidine-4-sulfonamide, potassium salt (Example 97) using 4-(4-isocyanato-2,3-dihydro-1H-inden-5-yl)picolinonitrile (Intermediate A43) and 1-ethylpiperidine-4-sulfonamide (Intermediate P11) to afford the title compound (18 mg, 18%) as a white solid.

¹H NMR (methanol-d₄) δ 8.66 (dd, 1H), 7.95 (d, 1H), 7.73 (dd, 1H), 7.20 (q, 2H), 3.55 (m, 1H), 3.09 (q, 2H), 2.98 (m, 4H), 2.85 (m, 4H), 2.13 (m, 2H), 2.1-1.97 (m, 4H), 1.31 (t, 3H).

LCMS: m/z 454 (M+H)⁺ (ES⁺); 452 (M−H)⁻ (ES⁻).

### Example 100 1-Ethyl-N-((4-fluoro-2-isopropyl-6-(2-methoxypyridin-4-yl) phenyl)carbamoyl)piperidine-4-sulfonamide, potassium salt

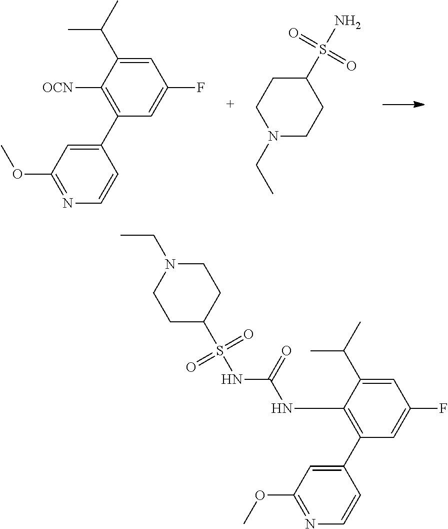

Prepared as described for 1-ethyl-N-((7-fluoro-5-(2-methoxypyridin-4-yl)-2,3-dihydro-1H-inden-4-yl)carbamoyl)piperidine-4-sulfonamide, potassium salt (Example 97) using 4-(5-fluoro-2-isocyanato-3-isopropylphenyl)-2-methoxypyridine (Intermediate A38) and 1-ethylpiperidine-4-sulfonamide (Intermediate P11) to afford the title compound (23 mg, 14%) as a white solid.

¹H NMR (methanol-d₄) δ 8.09 (d, 1H), 7.06 (dd, 2H), 6.88 (m, 2H), 3.92 (s, 3H), 3.72 (m, 1H), 3.19 (m, 1H), 3.08 (m, 2H), 2.49 (d, 2H), 1.87 (m, 6H), 1.23 (d, 6H), 1.12 (t, 3H).

LCMS: m/z 479 (M+H)⁺ (ES⁺); 477 (M−H)⁻ (ES⁻).

### Example 101: 1-Ethyl-N-((5-(pyridin-4-yl)-2,3-dihydro-1H-inden-4-yl) carbamoyl)piperidine-4-sulfonamide, potassium salt

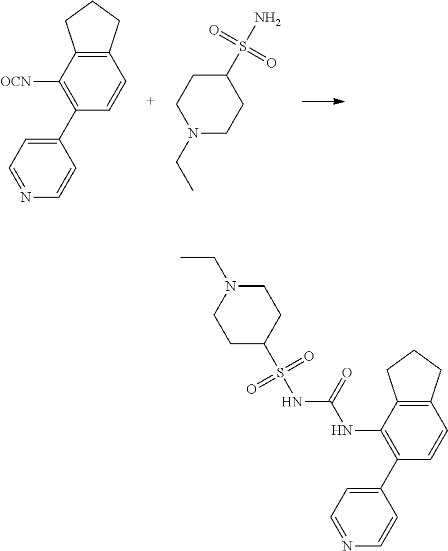

Prepared as described for 1-ethyl-N-((7-fluoro-5-(2-methoxypyridin-4-yl)-2,3-dihydro-1H-inden-4-yl)carbamoyl)piperidine-4-sulfonamide, potassium salt (Example 97) using 4-(4-isocyanato-2,3-dihydro-1H-inden-5-yl)pyridine (Intermediate A44) and 1-ethylpiperidine-4-sulfonamide (Intermediate P11) to afford the title compound (11 mg, 13%) as a white solid.

¹H NMR (methanol-d₄) δ 8.55-8.42 (m, 2H), 7.58-7.44 (m, 2H), 7.24-7.05 (m, 2H), 3.22 (d, 2H), 3.07 (m, 1H), 2.97 (m, 4H), 2.65 (t, 2H), 2.23 (t, 2H), 2.10 (m, 2H), 2.04-1.67 (m, 4H), 1.18 (t, 3H).

LCMS: m/z 429 (M+H)⁺ (ES⁺); 427 (M−H)⁻ (ES⁻).

### Example 102: N-((2-(2-Cyanopyridin-4-yl)-4-fluoro-6-isopropylphenyl)carbamoyl)-6-(dimethylamino)pyrazine-2-sulfonamide

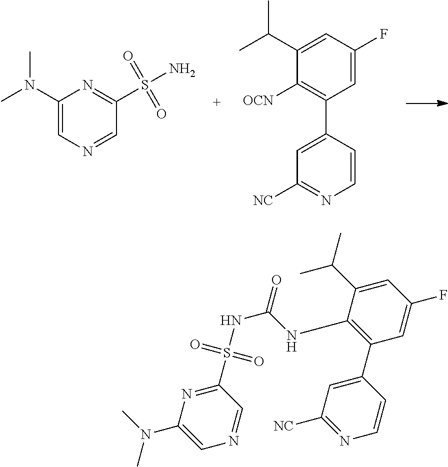

To a solution of 6-(dimethylamino)pyrazine-2-sulfonamide (intermediate P21) (65 mg, 321.41 μmol, 1 eq) in THF (2 mL) was added with t-BuONa (30 mg, 321.41 μmol, 1 eq). The mixture was stirred at 25° C. for 30 minutes. Then 4-(5-fluoro-2-isocyanato-3-isopropylphenyl)picolinonitrile (intermediate A37) (90 mg, 321.41 μmol, 1 eq) was added and the resulting mixture was stirred at 70° C. for 10 minutes. The reaction mixture was concentrated in vacuo. The residue was purified by prep-HPLC (column: Waters Xbridge C18, 150 mm*50 mm*10 μm; mobile phase: [A: water (0.05% ammonium hydroxide v/v); B: MeCN]; B %: 18%-48%, 11.5 min) to give the title compound (75.35 mg, 48% yield, 100% purity on LCMS) as a white solid.

¹H NMR (DMSO-d₆): δ 8.57 (d, 1H), 8.05 (s, 1H), 7.96 (s, 1H), 7.64 (br s, 1H), 7.20-7.14 (m, 4H), 3.19-3.15 (m, 1H), 3.07 (s, 6H) and 1.08 (d, 6H).

LCMS: m/z 484.2 (M+H)⁺ (ES⁺).

### Example 103: 6-(Dimethylamino)-N-((4-fluoro-2-isopropyl-6-(2-methoxypyridin-4-yl)phenyl)carbamoyl)pyrazine-2-sulfonamide

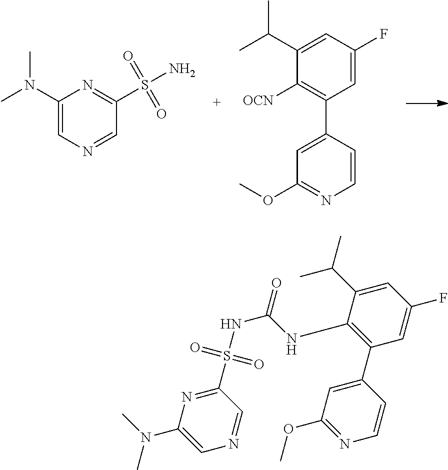

To a solution of 6-(dimethylamino) pyrazine-2-sulfonamide (intermediate P21) (65 mg, 321.41 μmol, 1 eq) in THF (2 mL) was added with t-BuONa (30 mg, 321.41 μmol, 1 eq). The mixture was stirred at 25° C. for 30 minutes. Then 4-(5-fluoro-2-isocyanato-3-isopropylphenyl)-2-methoxypyridine (intermediate A38) (92 mg, 321.41 μmol, 1 eq) was added. The mixture was stirred at 70° C. for 10 minutes and then concentrated in vacua. The residue was purified by prep-HPLC (column: Waters Xbridge C18, 150 mm*50 mm*10 μm; mobile phase: [A: water (10 mM NH₄HCO₃); B: MeCN]; B %: 20%-50%, 11.5 min) to give the title compound (41.48 mg, 26% yield, 100% purity on LCMS) as a white solid.

¹H NMR (DMSO-d₆): δ 8.27 (s, 1H), 8.10 (s, 1H), 8.05 (d, 1H), 7.74 (br s, 1H), 7.14 (d, 1H), 6.97 (d, 1H), 6.91 (s, 1H), 6.76 (s, 1H), 3.87 (s, 3H), 3.11 (s, 6H), 3.04-2.95 (m, 1H) and 1.25-1.02 (m, 6H).

LCMS: m/z 489.2 (M+H)⁺ (ES⁺).

### Example 104: 6-(Dimethylamino)-N-((5-(2-methoxypyridin-4-yl)-2,3-dihydro-1H-inden-4-yl)carbamoyl)pyrazine-2-sulfonamide

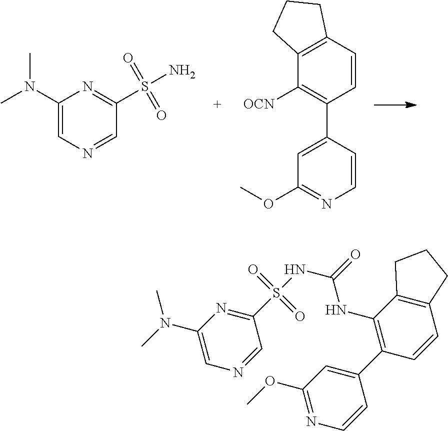

To a solution of 6-(dimethylamino) pyrazine-2-sulfonamide (intermediate P21) (65 mg, 321.41 μmol, 1 eq) in THF (2 mL) was added with t-BuONa (30 mg, 321.41 μmol, 1 eq). The mixture was stirred at 25° C. for 30 minutes. Then 4-(4-isocyanato-2,3-dihydro-1H-inden-5-yl)-2-methoxypyridine (intermediate A39) (85 mg, 321.41 μmol, 1 eq) was added. The mixture was stirred at 70° C. for 10 minutes and then concentrated in vacuo. The residue was purified by prep-HPLC (column: Waters Xbridge C18, 150 mm*50 mm*10 μm; mobile phase: [A: water (10 mM NH₄HCO₃); B: MeCN]; B %: 18%-48%, 11.5 min) to give the title compound (96.47 mg, 64% yield, 100% purity on LCMS) as a white solid.

¹H NMR (DMSO-d₆): δ 8.23 (s, 1H), 8.14 (s, 1H), 8.06 (d, 1H), 7.65 (br s, 1H), 7.13 (d, 1H), 7.06 (d, 1H), 6.90 (d, 1H), 6.74 (s, 1H), 3.87 (s, 3H), 3.09 (s, 6H), 2.89 (t, 2H), 2.71-2.67 (m, 2H) and 2.00-1.91 (m, 2H).

LCMS: m/z 469.2 (M+H)⁺ (ES⁺).

### Example 105: 6-(Dimethylamino)-N-((7-fluoro-5-(pyridin-4-yl)-2,3-dihydro-1H-inden-4-yl)carbamoyl)pyrazine-2-sulfonamide

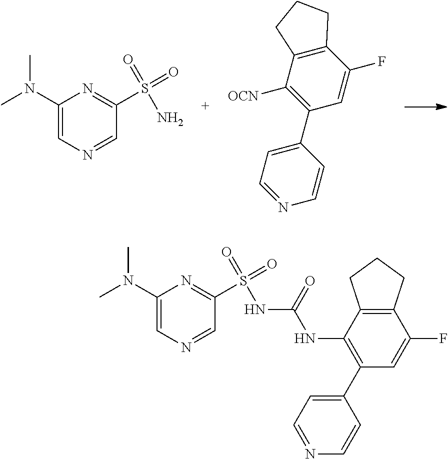

A mixture of 6-(dimethylamino)pyrazine-2-sulfonamide (intermediate P21) (60 mg, 296.69 μmol, 1 eq) and t-BuONa (29 mg, 296.69 μmol, 1 eq) in THF (2 mL) was stirred at 25° C. for 10 minutes. Then 4-(7-fluoro-4-isocyanato-2,3-dihydro-1H-inden-5-yl)pyridine (intermediate A40) (75 mg, 296.69 μmol, 1 eq) was added. The mixture was stirred at 25° C. for 10 minutes and then concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 10 min) to give the title compound (10 mg, 7% yield, 100% purity on LCMS) as a white solid.

¹H NMR (DMSO-d₆): δ 11.13 (br s, 1H), 8.50 (d, 2H), 8.30 (s, 1H), 8.15 (s, 1H), 7.83 (br s, 7.30 (d, 2H), 6.98 (d, 1H), 3.11 (s, 6H), 2.94 (t, 2H), 2.73-2.69 (m, 2H) and 2.08-2.00 (m, 2H).

LCMS: m/z 457.2 (M+H)⁺ (ES⁺).

### Example 106: N-((2-(2-Cyanopyridin-4-yl)-4-fluoro-6-isopropylphenyl)carbamoyl)-5-(dimethylamino)pyrazine-2-sulfonamide

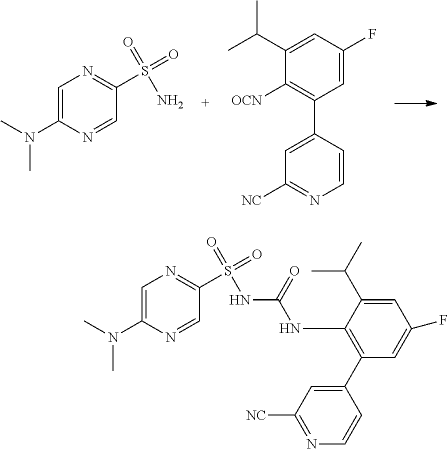

To a solution of 5-(dimethylamino)pyrazine-2-sulfonamide (intermediate P22) (60 mg, 296.69 μmol, 1 eq) in THF (4 mL) was added t-BuONa (29 mg, 296.69 μmol, 1 eq) and 4-(5-fluoro-2-isocyanato-3-isopropylphenyl)picolinonitrile (intermediate A37) (83 mg, 296.69 μmol, 1 eq). The mixture was stirred at 25° C. for 30 minutes and then concentrated under reduced pressure. The residue was purified by prep-HPLC (column: Phenomenex Gemini C18, 150 mm*25 mm*10 μm; mobile phase: [A: water (0.05% ammonium hydroxide v/v); B: MeCN]; B %: 5%-35%, 11.5 min) to give the title compound (49 mg, 34% yield, 100% purity on LCMS) as a white solid.

¹H NMR (DMSO-d₆): δ 8.58 (d, 1H), 8.24 (s, 7.99 (s, 7.92 (s, 7.78 (br s, 1H), 7.60 (s, 1H), 7.20 (dd, 1H), 7.06 (dd, 1H), 3.18 (s, 6H), 3.14-1.09 (m, 1H) and 1.10 (d, 6H).

LCMS: m/z 484.2 (M+H)⁺ (ES⁺).

### Example 107: 5-(Dimethylamino)-N-((4-fluoro-2-isopropyl-6-(2-methoxypyridin-4-yl)phenyl)carbamoyl)pyrazine-2-sulfonamide

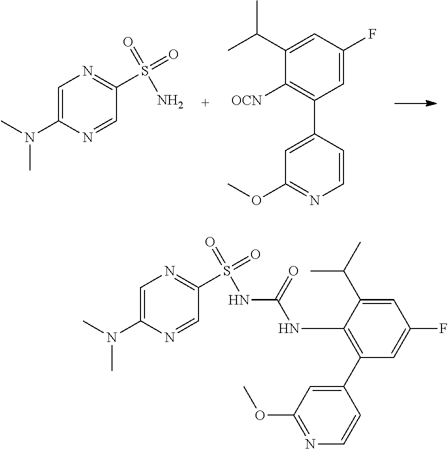

To a solution of 5-(dimethylamino)pyrazine-2-sulfonamide (intermediate P22) (71 mg, 349.28 μmol, 1 eq) in THF (5 mL) was added t-BuONa (34 mg, 349.28 μmol, 1 eq) and 4-(5-fluoro-2-isocyanato-3-isopropylphenyl)-2-methoxypyridine (intermediate A38) (100 mg, 349.28 μmol, 1 eq). The mixture was stirred at 25° C. for 30 minutes and then concentrated under reduced pressure. The residue was purified by prep-HPLC (column: Phenomenex Gemini C18, 150 mm*25 mm*5 μm; mobile phase: [A: water (0.05% ammonium hydroxide v/v); B: MeCN]; B %: 0%-30%, 10 min) to give the title compound (30 mg, 18% yield, 100% purity on LCMS) as a white solid.

¹H NMR (DMSO-d₆): δ 8.40 (s, 1H), 8.12 (s, 1H), 8.06 (d, 1H), 7.73 (br s, 1H), 7.16 (dd, 1H), 6.99-6.96 (m, 1H), 6.82 (d, 1H), 6.72 (s, 1H), 3.87 (s, 3H), 3.18 (s, 6H), 2.95-2.91 (m, 1H) and 1.12-0.95 (m, 6H).

LCMS: m/z 489.3 (M+H)⁺ (ES⁺).

### Example 108: 5-(Dimethylamino)-N-((5-(2-methoxypyridin-4-yl)-2,3-dihydro-1H-inden-4-yl)carbamoyl)pyrazine-2-sulfonamide

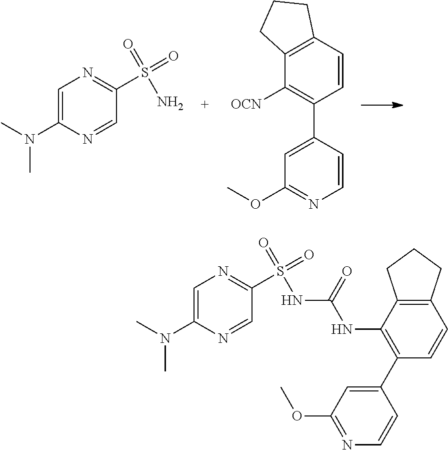

To a solution of 5-(dimethylamino)pyrazine-2-sulfonamide (intermediate P22) (70 mg, 346.13 μmol, 1 eq) in THF (5 mL) was added t-BuONa (33 mg, 346.13 μmol, 1 eq) and 4-(4-isocyanato-2,3-dihydro-1H-inden-5-yl)-2-methoxypyridine (intermediate A39) (92 mg, 346.13 μmol, 1 eq). The mixture was stirred at 25° C. for 30 minutes and then concentrated under reduced pressure. The residue was purified by prep-HPLC (column: Phenomenex Gemini C18, 150 mm*25 mm*10 μm; mobile phase: [A: water (0.05% ammonium hydroxide v/v); B: MeCN]; B %: 2%-32%, 11.5 min) to give the title compound (40 mg, 24% yield, 98.92% purity on LCMS) as a white solid.

¹H NMR (DMSO-d₆): δ 8.46-8.41 (m, 1H), 8.09-8.07 (t, 2H), 7.60 (br s, 1H), 7.13 (d, 1H), 7.05 (d, 1H), 6.82 (d, 1H), 6.68 (s, 1H), 3.86 (s, 3H), 3.16 (s, 6H), 2.88 (t, 2H), 2.65 (t, 2H) and 1.99-1.91 (m, 2H).

LCMS: m/z 469.3 (M+H)⁺ (ES⁺).

### Example 109: 5-(Dimethylamino)-N-((7-fluoro-5-(pyridin-4-yl)-2,3-dihydro-1H-inden-4-yl)carbamoyl)pyrazine-2-sulfonamide

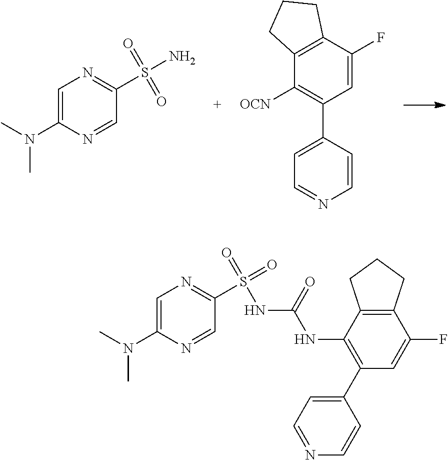

To a mixture of 5-(dimethylamino)pyrazine-2-sulfonamide (intermediate P22) (80 mg, 393.30 μmol, 1 eq) in THF (5 mL) was added t-BuONa (41 mg, 432.63 μmol, 1.1 eq) in one portion at 15° C. Then the reaction mixture was stirred for 15 minutes. Then a solution of 4-(7-fluoro-4-isocyanato-2,3-dihydro-1H-inden-5-yl)pyridine (intermediate A40) (100 mg, 393.30 μmol, 1 eq) in THF (2 mL) was added. The resulting mixture was stirred at 15° C. for 30 minutes and then concentrated under reduced pressure. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, to min) to give the title compound (72.57 mg, 40%) as an off-white solid.

¹H NMR (DMSO-d₆): δ 8.49 (d, 2H), 8.40 (s, 1H), 8.07 (s, 1H), 7.54 (br s, 1H), 7.28 (d, 2H), 6.93 (d, 1H), 3.16 (s, 6H), 2.93 (t, 2H), 2.74 (t, 2H) and 2.07-1.99 (m, 2H).

LCMS: m/z 457.2 (M+H)⁺ (ES⁺).

### Example 110: N-((2-(2-Cyanopyridin-4-yl)-4-fluoro-6-isopropylphenyl)carbamoyl)-3-(difluoromethyl)pyrazine-2-sulfonamide

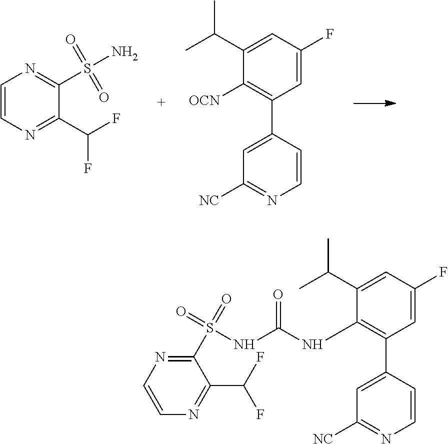

To a solution of 3-(difluoromethyl)pyrazine-2-sulfonamide (intermediate P23) (74 mg, 355.5 μmol, 1 eq) in THF (4 mL) was added t-BuONa (34 mg, 355.51 μmol, 1 eq) and 4-(5-fluoro-2-isocyanato-3-isopropylphenyl)picolinonitrile (intermediate A37) (0.1 g, 355.51 μmol, 1 eq). The mixture was stirred at 25° C. for 10 minutes and then concentrated in vacuo. The residue was purified by prep-HPLC (column: Phenomenex Gemini C18, 150 mm*25 mm*10 μm; mobile phase: [A: water (0.05% NH₄HCO₃); B: MeCN]; B %: 20%-50%, 12 min) to give the title compound (13.20 mg, 7% yield, 98.3% purity on LCMS) as a white solid.

¹H NMR (DMSO-d₆+D₂O): δ 8.75-8.61 (m, 2H), 8.45 (d, 1H), 7.95-7.59 (m, 2H), 7.48 (d, 1H), 7.19-7.13 (m, 1H), 7.12-6.95 (m, 1H), 3.20-3.04 (m, 1H) and 1.19-0.93 (m, 6H).

LCMS: m/z 491.2 (M+H)⁺ (ES⁺).

### Example 111: 3-(Difluoromethyl)-N-((4-fluoro-2-isopropyl-6-(2-methoxypyridin-4-yl)phenyl)carbamoyl)pyrazine-2-sulfonamide

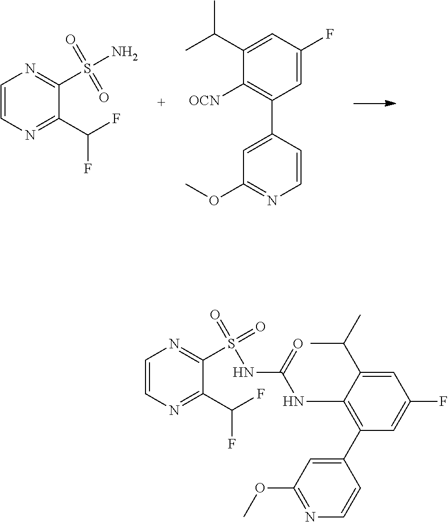

To a solution of 3-(difluoromethyl)pyrazine-2-sulfonamide (intermediate P23) (73 mg, 349.28 μmol, 1 eq) in THF (4 mL) was added t-BuONa (34 mg, 349.28 μmol, 1 eq) and 4-(5-fluoro-2-isocyanato-3-isopropylphenyl)-2-methoxypyridine (intermediate A38) (100 mg, 349.28 μmol, 1 eq). The mixture was stirred at 25° C. for 10 minutes and then concentrated in vacuo. The residue was purified by prep-HPLC (column: Phenomenex Gemini C18, 150 mm*25 mm*10 μm; mobile phase: [A: water (0.05% NH₄HCO₃); B: MeCN]; B %: 17%-47%, 12 min) to give the title compound (14.57 mg, 8% yield, 98.6% purity on LCMS) as a white solid.

¹H NMR (DMSO-d₆+D₂O): δ 8.82-8.76 (m, 2H), 7.98-7.65 (m, 2H), 7.15-7.00 (m, 1H), 6.88-6.86 (m, 1H), 6.79 (d, 1H), 6.61 (s, 1H), 3.82-3.79 (m, 3H), 3.19-2.93 (m, 1H) and 1.21-0.97 (m, 6H).

LCMS: m/z 496.2 (M+H)⁺ (ES⁺).

### Example 112: 3-(Difluoromethyl)-N-((5-(2-methoxypyridin-4-yl)-2,3-dihydro-1H-inden-4-yl)carbamoyl)pyrazine-2-sulfonamide

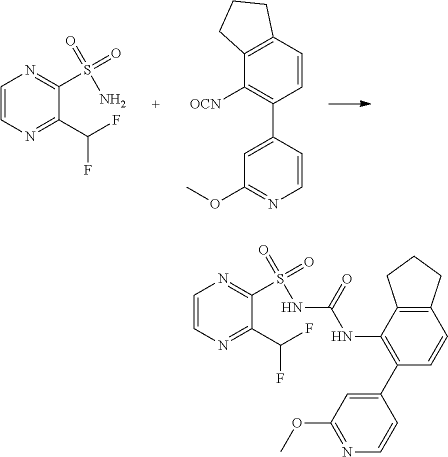

To a solution of 3-(difluoromethyl)pyrazine-2-sulfonamide (intermediate P23) (75 mg, 358.55 μmol, 1 eq) in THF (5 mL) was added t-BuONa (34 mg, 358.55 μmol, 1 eq) and 4-(4-isocyanato-2,3-dihydro-1H-inden-5-yl)-2-methoxypyridine (intermediate A39) (95 mg, 358.55 μmol, 1 eq). The mixture was stirred at 10° C. for 1 hour and then concentrated in vacuo. The residue was purified by prep-HPLC (column: Phenomenex Gemini C18, 150 mm*25 mm*10 μm; mobile phase: [A: water (10 mM NH₄HCO₃); B: MeCN]; B %: 15%-45%, 12 Min) to give the title compound (24.17 mg, 14% yield, 100% purity on LCMS) as a white solid.

¹H NMR (DMSO-d₆): δ 8.78 (s, 2H), 8.15-7.87 (m, 2H), 7.07 (d, 1H), 7.00 (d, 1H), 6.85-6.83 (m, 1H), 6.67 (s, 1H), 6.06 (br s, 1H), 3.85 (s, 3H), 2.88-2.84 (m, 2H), 2.68-2.63 (m, 2H) and 1.96-1.90 (m, 2H).

LCMS: m/z 476.2 (M+H)⁺ (ES^{+).}

### Example 113: 3-(Difluoromethyl)-N-((7-fluoro-5-(pyridin-4-yl)-2,3-dihydro-1H-inden-4-yl)carbamoyl)pyrazine-2-sulfonamide

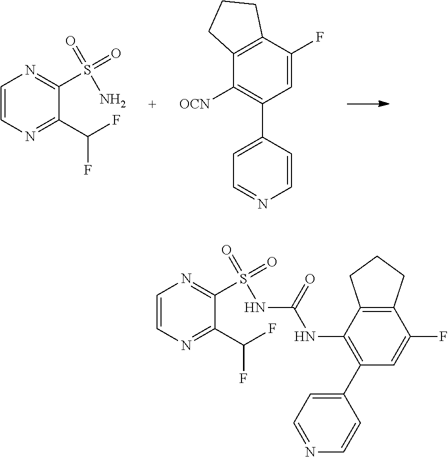

To a solution of 3-(difluoromethyl)pyrazine-2-sulfonamide (intermediate P23) (82.27 mg, 393.30 μmol, 1 eq) in THF (5 mL) was added t-BuONa (42 mg, 432.63 μmol, 1.1 eq) and a solution of 4-(7-fluoro-4-isocyanato-2,3-dihydro-1H-inden-5-yl)pyridine (intermediate A40) (100 mg, 393.30 μmol, 1 eq) in THF (5 mL) and DCM (5 mL). The reaction mixture was stirred at 16° C. for 0.5 hour and then concentrated in vacuo. The residue was purified by prep-HPLC (column: Phenomenex Gemini C18, 150 mm*25 mm*10 μm; mobile phase: [A: water (0.05% NH₄HCO₃); B: MeCN]; B %: 15%-45%, 10 min) to give the title compound (25.31 mg, 14%) as a light yellow solid.

¹H NMR (DMSO-d6+D₂O): δ 8.89 (s, 1H), 8.85 (d, 1H), 8.49 (d, 2H), 7.76 (t, 1H), 7.45-7.25 (m, 2H), 6.96 (d, 1H), 2.92 (t, 2H), 2.72-2.67 (m, 2H) and 2.05-2.01 (m, 2H).

LCMS: m/z 464.1 (M+H)⁺ (ES⁺).

### Example 114: N-((2-(2-Cyanopyridin-4-yl)-4-fluoro-6-isopropylphenyl)carbamoyl)-4,6-dimethylpyrimidine-2-sulfonamide

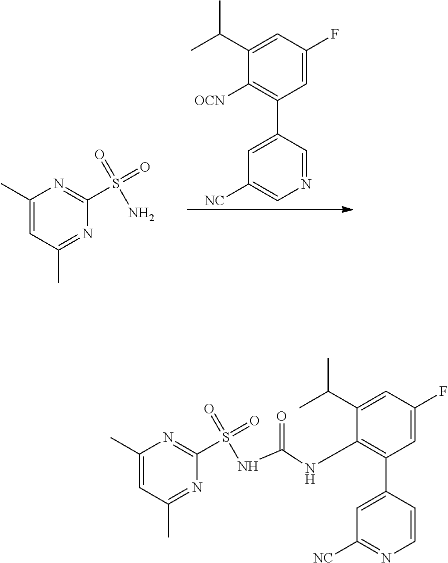

To a mixture of 4,6-dimethylpyrimidine-2-sulfonamide (intermediate P24) (65 mg, 347.19 μmol, 1 eq) in THF (5 mL) was added t-BuONa (33 mg, 347.19 μmol, 1 eq) in one portion at 25° C. under N₂. Then the reaction mixture was stirred for 10 minutes. Then 4-(5-fluoro-2-isocyanato-3-isopropylphenyl)picolinonitrile (intermediate A37) (98 mg, 347.19 μmol, 1 eq) was added. The resulting mixture was heated to 70° C. and stirred for 10 minutes. The reaction mixture was concentrated in vacua. The residue was purified by prep-HPLC (column: Phenomenex Gemini C18, 150 mm*25 mm*10 μm; mobile phase: [A: water (10 mM NH₄HCO₃); B: MeCN]; B %: 12%-42%, 10 min) to give the title compound (19.94 mg, 12% yield, 100% purity on LCMS) as a white solid.

¹H NMR (DMSO-d₆): δ 8.69-8.68 (m, 1H), 8.02 (s, 1H), 7.71-7.69 (m, 1H), 7.35-7.33 (m, 1H), 7.25-7.20 (m, 1H), 7.13-7.09 (m, 2H), 3.33-3.16 (m, 1H), 2.43 (s, 6H) and 1.10 (d, 6H).

LCMS: m/z 469.2 (M+H)⁺ (ES⁺).

### Example 115: N-((4-Fluoro-2-isopropyl-6-(2-methoxypyridin-4-yl)phenyl)carbamoyl)-4,6-dimethylpyrimidine-2-sulfonamide

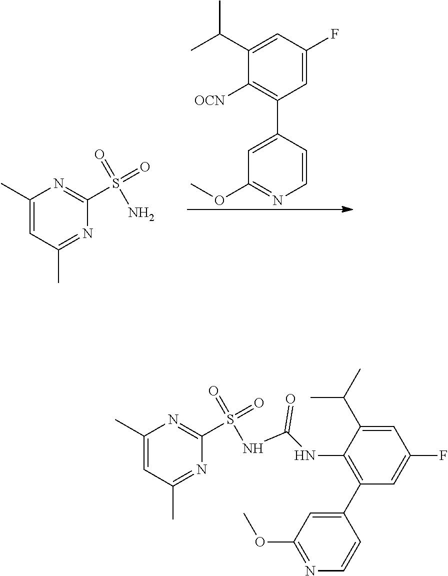

To a mixture of 4,6-dimethylpyrimidine-2-sulfonamide (intermediate P24) (65 mg, 349.28 μmol, 1 eq) in THF (5 mL) was added t-BuONa (34 mg, 349.28 μmol, 1 eq) in one portion at 25° C. under N₂. Then the reaction mixture was stirred for 10 minutes. Then 4-(5-fluoro-2-isocyanato-3-isopropylphenyl)-2-methoxypyridine (intermediate A38) (100 mg, 349.28 μmol, 1 eq) was added. The reaction mixture was heated to 70° C. and stirred for 10 minutes. The reaction mixture was concentrated in vacuo. The residue was purified by prep-HPLC (column: Waters Xbridge C18, 150 mm*50 mm*10 μm; mobile phase: [A: water (0.05% ammonium hydroxide v/v); B: MeCN]; B %: 5%-35%, 11.5 min) to give the title compound (60.47 mg, 37% yield, 100% purity on LCMS) as a white solid.

¹H NMR (DMSO-d₆): δ 8.11-8.07 (m, 1H), 7.85 (br s, 1H), 7.42-7.39 (m, 1H), 7.18-7.12 (m, 1H), 7.05-6.94 (m, 2H), 6.76 (s, 1H), 3.90 (s, 3H), 3.12-3.08 (m, 1H), 2.46 (s, 6H) and 1.14-1.07 (m, 6H).

LCMS: m/z 474.2 (M+H)⁺ (ES⁺).

### Example 116: N-((5-(2-Methoxypyridin-4-yl)-2,3-dihydro-1H-inden-4-yl)carbamoyl)-4,6-dimethylpyrimidine-2-sulfonamide

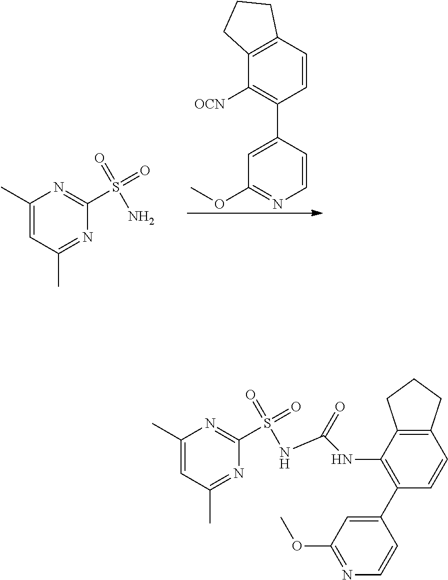

To a mixture of 4,6-dimethylpyrimidine-2-sulfonamide (intermediate P24) (70 mg, 375.52 μmol, 1 eq) in THF (5 mL) was added t-BuONa (36 mg, 375.52 μmol, 1 eq) in one portion at 25° C. under N₂. Then the reaction mixture was stirred for 10 minutes. Then 4-(4-isocyanato-2,3-dihydro-1H-inden-5-yl)-2-methoxypyridine (intermediate A39) (100 mg, 375.52 μmol, 1 eq) was added. The reaction mixture was heated to 70° C. and stirred for 10 minutes. The reaction mixture was concentrated in vacuo. The residue was purified by prep-HPLC (column: Waters Xbridge C18, 150 mm*50 mm*10 μm; mobile phase: [A: water (0.05% ammonium hydroxide v/v); B: MeCN]; B %: 2%-32%, 11.5 min) to give the title compound (41.33 mg, 24% yield, 98.29% purity on LCMS) as a white solid.

¹H NMR (DMSO-d₆): δ 8.10 (d, 1H), 7.32-7.30 (m, 1H), 7.11 (d, 1H), 7.05 (d, 1H), 6.98 (d, 1H), 6.76 (s, 1H), 3.86 (s, 3H), 2.87 (t, 2H), 2.76-2.73 (m, 2H), 2.49 (s, 6H) and 1.98-1.93 (m, 2H).

LCMS: m/z 454.2 (M+H)⁺ (ES⁺).

### Example 117: N-((7-Fluoro-5-(pyridin-4-yl)-2,3-dihydro-1H-inden-4-yl)carbamoyl)-4,6-dimethylpyrimidine-2-sulfonamide

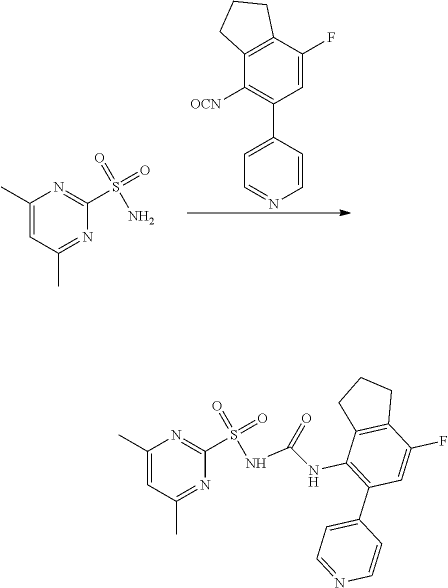

To a mixture of 4,6-dimethylpyrimidine-2-sulfonamide (intermediate P24) (50 mg, 267.07 μmol, 1 eq) in THF (3 mL) was added t-BuONa (26 mg, 267.07 μmol, 1 eq) in one portion at 25° C. under N₂. Then the reaction mixture was stirred for 10 minutes. Then 4-(7-fluoro-4-isocyanato-2,3-dihydro-1H-inden-5-yl)pyridine (intermediate A40) (68 mg, 267.07 μmol, 1 eq) was added. The reaction mixture was stirred at 25° C. for 10 minutes and then concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 10 min) to give the title compound (22.84 mg, 19% yield, 97.11% purity on LCMS) as a white solid.

¹H NMR (DMSO-d₆): δ 8.56 (d, 2H), 7.75 (br s, 1H), 7.39-7.36 (m, 3H), 6.98 (d, 1H), 2.93 (t, 2H), 2.85-2.75 (m, 2H), 2.49 (s, 6H) and 2.06-2.02 (m, 2H).

LCMS: m/z 442.1 (M+H)⁺ (ES⁺).

### Example 118: N-((2-(2-Cyanopyridin-4-yl)-4-fluoro-6-isopropylphenyl)carbamoyl)-5-(dimethylamino)pyridazine-3-sulfonamide

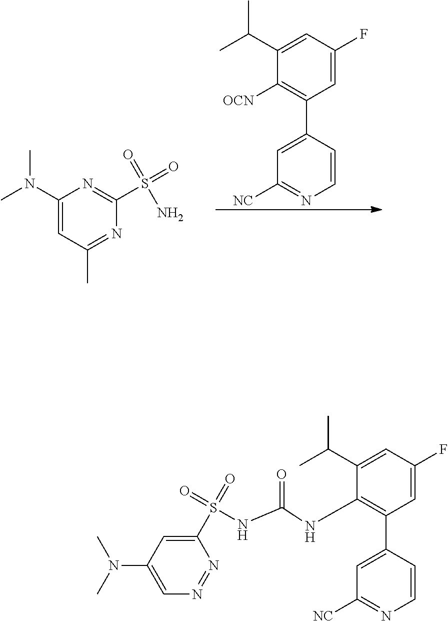

To a mixture of 5-(dimethylamino)pyridazine-3-sulfonamide (intermediate P25) (70 mg, 346.13 μmol, 1 eq) in THF (2 mL) was added t-BuONa (33 mg, 346.13 μmol, 1 eq) in one portion at 25° C. under N₂. Then the reaction mixture was stirred for 10 minutes. Then 4-(5-fluoro-2-isocyanato-3-isopropylphenyl)picolinonitrile (intermediate A37) (97 mg, 346.13 μmol, 1 eq) was added. The reaction mixture was stirred at 25° C. for 10 minutes and then concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 10 min) to give the title compound (65.88 mg, 39% yield, 99.38% purity on LCMS) as a white solid.

¹H NMR (DMSO-d₆): δ 8.77 (d, 1H), 8.61-8.59 (m, 1H), 7.94 (s, 1H), 7.87-7.84 (m, 1H), 7.59-7.58 (m, 1H), 7.20-7.17 (m, 1H), 7.07 (dd, 1H), 6.96 (s, 1H), 3.21-3.17 (m, 1H), 3.09 (s, 6H) and 1.15-1.08 (m, 6H).

LCMS: m/z 484.2 (M+H)⁺ (ES⁺).

### Example 119: 5-(Dimethylamino)-N-((4-fluoro-2-isopropyl-6-(2-methoxypyridin-4-yl)phenyl) carbamoyl) pyridazine-3-sulfonamide

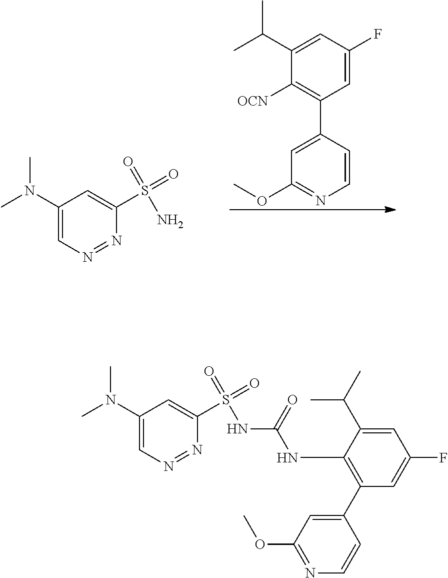

To a mixture of 5-(dimethylamino)pyridazine-3-sulfonamide (intermediate P25) (40 mg, 197.79 μmol, 1 eq) in THF (5 mL) was added t-BuONa (19 mg, 197.79 μmol, 1 eq) in one portion at 25° C. under N₂. Then the reaction mixture was stirred for 10 minutes. Then 4-(5-fluoro-2-isocyanato-3-isopropylphenyl)-2-methoxypyridine (intermediate A38) (57 mg, 197.79 μmol, 1 eq) was added. The resulting mixture was heated to 70° C. and stirred for 10 minutes. The reaction mixture was concentrated in vacuo. The residue was purified by prep-HPLC (column: Phenomenex Gemini C18, 150 mm*25 mm*10 μm; mobile phase: [A: water (10 mM NH₄HCO₃); B: MeCN]; B %: 13%-43%, 10 min) to give the title compound (49.52 mg, 51% yield, 98.93% purity on LCMS) as a white solid.

¹H NMR (DMSO-d₆): δ 8.90-8.85 (m, 1H), 8.09-8.05 (m, 1H), 7.92-7.87 (m, 1H), 7.18-7.15 (m, 1H), 7.07 (d, 1H), 6.98 (d, 1H), 6.84 (d, 1H), 6.73 (s, 1H), 3.85 (s, 3H), 3.07 (s, 6H), 3.06-3.01 (m, 1H) and 1.09-0.94 (m, 6H).

LCMS: m/z 489.2 (M+H)⁺ (ES⁺).

### Example 120: 5-(Dimethylamino)-N-((5-(2-methoxypyridin-4-yl)-2,3-dihydro-1H-inden-4-yl) carbamoyl) pyridazine-3-sulfonamide

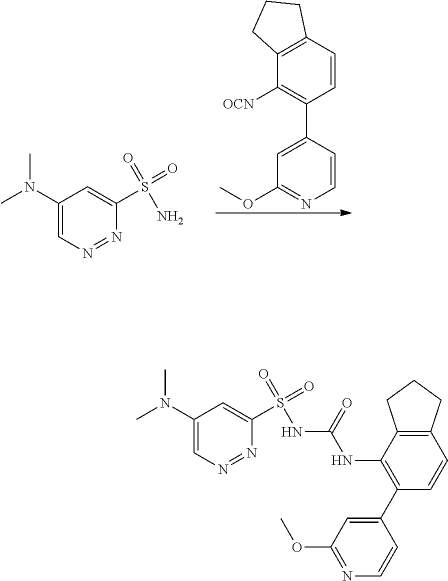

To a mixture of 5-(dimethylamino)pyridazine-3-sulfonamide (intermediate P25) (35 mg, 173.07 μmol, 1 eq) in THF (2 mL) was added t-BuONa (17 mg, 173.07 μmol, 1 eq) in one portion at 25° C. under N₂. Then the reaction mixture was stirred for 10 minutes. Then 4-(4-isocyanato-2,3-dihydro-1H-inden-5-yl)-2-methoxypyridine (intermediate A39) (46 mg, 173.07 μmol, 1 eq) was added. The reaction mixture was heated to 25° C. and stirred for 20 minutes. The reaction mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 10 min) to give the title compound (21.73 mg, 27% yield, 99.14% purity on LCMS) as a white solid.

¹H NMR (DMSO-d₆): δ 8.83 (d, 1H), 8.06 (d, 1H), 7.75-7.74 (m, 1H), 7.13 (d, 1H), 7.07-7.05 (m, 2H), 6.86 (d, 1H), 6.71 (s, 1H), 3.88 (s, 3H), 3.06 (s, 6H), 2.86 (t, 2H), 2.68 (t, 2H) and 1.99-1.93 (m, 2H).

LCMS: m/z 469.2 (M+H)⁺ (ES⁺).

### Example 121: 5-(Dimethylamino)-N-((7-fluoro-5-(pyridin-4-yl)-2,3-dihydro-1H-inden-4-yl) carbamoyl) pyridazine-3-sulfonamide

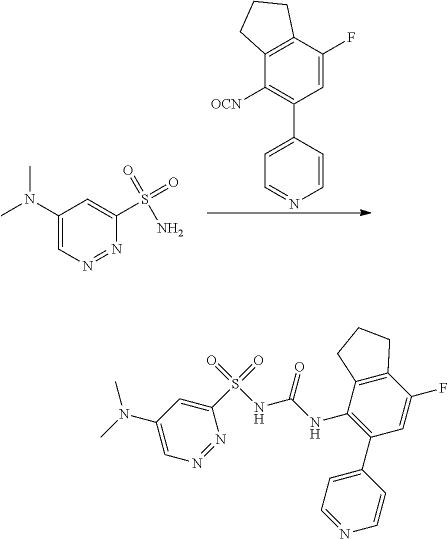

To a mixture of 5-(dimethylamino)pyridazine-3-sulfonamide (intermediate P25) (50 mg, 247.24 μmol, 1 eq) in THF (3 mL) was added t-BuONa (24 mg, 247.24 μmol, 1 eq) in one portion at 25° C. under N₂. Then the reaction mixture was stirred for 10 minutes. Then 4-(7-fluoro-4-isocyanato-2,3-dihydro-1H-inden-5-yl)pyridine (intermediate A40) (63 mg, 247.24 μmol, 1 eq) was added. The reaction mixture was stirred at 25° C. for 10 minutes. The reaction mixture was concentrated in vacua. The residue was purified by prep-HPLC (column: Phenomenex Gemini C18, 150 mm*25 mm*5 μm; mobile phase: [A: water (0.05% ammonium hydroxide v/v); B: MeCN]; B %: 5%-35%, 10 min) to give the title compound (22.81 mg, 20% yield, 98.41% purity on LCMS) as a white solid.

¹H NMR (DMSO-d₆): δ 8.83 (d, 1H), 8.51 (d, 2H), 7.71 (br s, 1H), 7.31-7.30 (m, 2H), 7.04 (d, 1H), 6.95 (d, 1H), 3.06 (s, 6H), 2.92 (t, 2H), 2.78-2.75 (m, 2H) and 2.05-2.00 (m, 2H).

LCMS: m/z 457.0 (M+H)⁺ (ES⁺).

### Example 122: 3-(N-Methyl-N-(1-methylpyrrolidin-3-yl)sulfamoyl)-1-(5-(2-methoxypyridin-4-yl)-2,3-dihydro-1H-inden-4-yl)urea

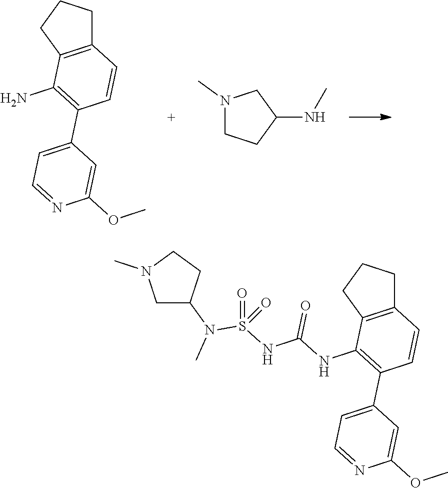

To a cooled (0° C.) solution of chlorosulfonyl isocyanate (59 mg, 0.41 mmol) in DCM (5 mL) was added 5-(2-methoxypyridin-4-yl)-2,3-dihydro-1H-inden-4-amine (Intermediate A35; 100 mg, 0.41 mmol). The mixture was stirred for 10 minutes at 0° C. N,1-dimethylpyrrolidin-3-amine (95 mg, 0.83 mmol) in DCM (5 mL) was added and the reaction was allowed to reach room temperature over 30 minutes. The mixture was evaporated to dryness in vacua and purified by reversed phase chromatography to afford the title compound (9 mg; 5%) as a white solid.

¹H NMR (CD₃OD) δ 8.12 (d, 1H), 7.19 (d, 1H), 7.13 (d, 1H), 6.99 (d, 1H), 6.83 (s, 1H), 4.48 (m, 1H), 3.92 (s, 3H), 2.92 (m, 6H), 2.82 (m, 2H), 2.71 (s, 3H), 2.50 (s, 3H), 2.10 (m, 3H) and 1.92 (m, 1H).

LCMS: m/z 460 (M+H)⁺ (ES⁺); 458 (M−H)⁻ (ES⁻).

### Example 123: 3-(N-Methyl-N-((1-methylpyrrolidin-2-yl)methyl) sulfamoyl)-1-(5-(2-methoxypyridin-4-yl)-2,3-dihydro-1H-inden-4-yl)urea

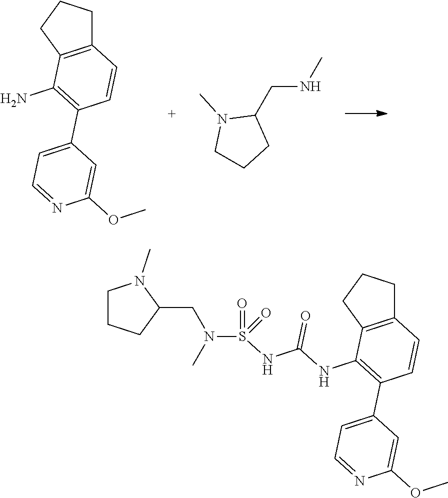

Prepared as described for 3-(N-methyl-N-(1-methylpyrrolidin-3-yl)sulfamoyl)-1-(5-(2-methoxypyridin-4-yl)-2,3-dihydro-1H-inden-4-yl)urea (Example 122), using chlorosulfonyl isocyanate (59 mg, 0.41 mmol), 5-(2-methoxypyridin-4-yl)-2,3-dihydro-1H-inden-4-amine (Intermediate A35; 100 mg, 0.41 mmol) and N-methyl-1-(1-methylpyrrolidin-2-yl)methanamine (107 mg, 0.83 mmol) to afford the title compound (2 mg; 1%) as a white solid.

¹H NMR (CD₃OD) δ 8.12 (d, 1H), 7.19 (m, 2H), 7.09 (d, 1H), 6.93 (s, 1H), 3.92 (s, 3H), 3.88 (m, 1H), 3.65 (m, 1H), 3.09 (m, 1H), 2.98 (m, 6H), 2.79 (s, 3H), 2.69 (s, 3H), 2.10 (m, 3H), 1.97 (m, 2H) and 1.60 (m, 1H).

LCMS: m/z 474 (M+H)⁺ (ES⁺).

### Example 124: 3-(N-Methyl-N-((1-methylpyrrolidin-2-yl)methyl) sulfamoyl)-1-(7-fluoro-5-(2-methoxypyridin-4-yl)-2,3-dihydro-1H-inden-4-yl)urea

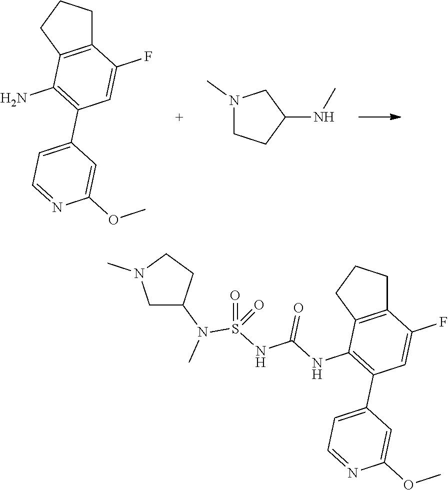

Prepared as described for 3-(N-methyl-N-(1-methylpyrrolidin-3-yl)sulfamoyl)-1-(5-(2-methoxypyridin-4-yl)-2,3-dihydro-1H-inden-4-yl)urea (Example 122), using chlorosulfonyl isocyanate (55 mg, 0.38 mmol), 7-fluoro-5-(2-methoxypyridin-4-yl)-2,3-dihydro-1H-inden-4-amine (Intermediate A34; 100 mg, 0.38 mmol) and N,1-dimethylpyrrolidin-3-amine (95 mg, 0.83 mmol) to afford the title compound (12 mg; 10%) as a white solid.

¹H NMR (CD₃OD) δ 8.14 (d, 1H), 7.08 (d, 1H), 6.98 (m, 2H), 4.48 (m, 1H), 3.92 (s, 3H), 2.98 (m, 8H), 2.71 (s, 3H), 2.60 (s, 3H), 2.10 (m, 3H) and 1.92 (m, 1H).

LCMS: m/z 479 (M+H)⁺ (ES^{+).}

### Example 125: 3-(N-Methyl-N-((1-methylpyrrolidin-2-yl)methyl) sulfamoyl)-1-(7-fluoro-5-(2-methoxypyridin-4-yl)-2,3-dihydro-1H-inden-4-yl)urea

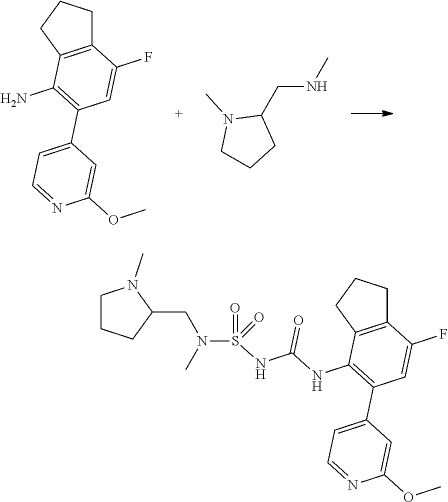

Prepared as described for 3-(N-methyl-N-(1-methylpyrrolidin-3-yl)sulfamoyl)-1-(5-(2-methoxypyridin-4-yl)-2,3-dihydro-1H-inden-4-yl)urea (Example 122), using chlorosulfonyl isocyanate (55 mg, 0.38 mmol), 7-fluoro-5-(2-methoxypyridin-4-yl)-2,3-dihydro-1H-inden-4-amine (Intermediate A34; 100 mg, 0.38 mmol) and N-methyl-1-(1-methylpyrrolidin-2-yl)methanamine (139 mg, 1.16 mmol) to afford the title compound (23 mg; 12%) as a white solid.

¹H NMR (CD₃OD) δ 8.12 (d, 1H), 7.00 (d, 1H), 6.90 (d, 1H), 6.83 (s, 1H), 3.92 (s, 3H), 3.78 (m, 1H), 3.55 (m, 1H), 3.00 (m, 7H), 2.79 (s, 3H), 2.67 (s, 3H), 2.19 (m, 3H), 2.01 (m, 2H) and 1.62 (m, 1H).

LCMS: m/z 492 (M+H)⁺ (ES⁺); 490 (M−H)⁻ (ES⁻).

### Example 126: (1R,4R)—N-((7-Fluoro-5-(2-methoxypyridin-4-yl)-2,3-dihydro-1H-inden-4-yl)carbamoyl)-5-methyl-2,5-diazabicyclo[2.2.1]heptane-2-sulfonamide

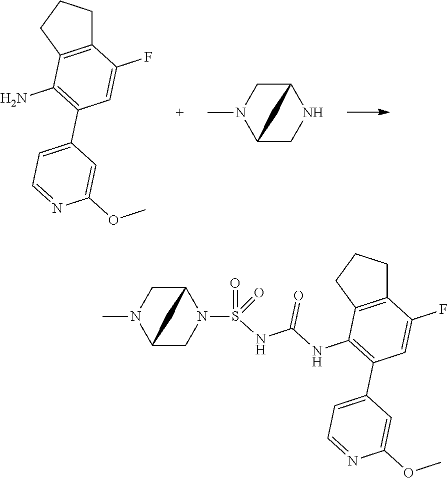

(1R,4R)-2-Methyl-2,5-diazabicyclo[2.2.1]heptane dihydrobromide (50 mg, 0.18 mmol) and sodium hydride (60%) (150 mg, 3.7 mmol) were refluxed for 1 hour in THF (10 mL). The mixture was cooled to room temperature and filtered over Celite. The filtrate was evaporated to dryness in vacua and the residue was dissolved in DCM (10 mL), after which DABCO was added (20 mg, 0.18 mmol).

Meanwhile, to a cooled (0° C.) solution of chlorosulfonyl isocyanate (35 mg, 0.25 mmol) in DCM (5 mL) was added 7-fluoro-5-(2-methoxypyridin-4-yl)-2,3-dihydro-1H-inden-4-amine (Intermediate A34; 66 mg, 0.26 mmol). The mixture was stirred for 10 minutes at 0° C.

Both DCM mixtures were combined and allowed to reach room temperature after 1 hour. The mixture was evaporated to dryness in vacua and purified by reversed phase chromatography to afford the title compound (4 mg; 5%) as a white solid.

¹H NMR (CD₃OD) δ 8.12 (d, 1H), 7.02 (d, 1H), 6.90 (m, 2H), 4.54 (m, 1H), 4.24 (m, 1H), 3.92 (s, 3H), 3.39 (m, 2H), 2.98 (m, 4H), 0.75 (s, 3H), 2.20 (m, 2H), and 1.64 (m, 2H).

LCMS: m/z 476 (M+H)⁺ (ES⁺); 474 (M−H)⁻ (ES⁻).

### Example 127: N-((2-(2-Cyanopyridin-4-yl)-4-fluoro-6-isopropylphenyl) carbamoyl)-1-phenylmethanesulfonamide

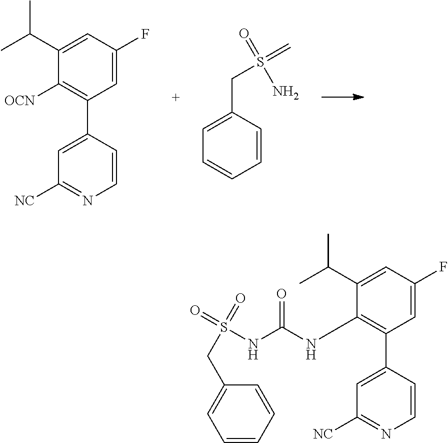

To a solution of phenylmethanesulfonamide (61 mg, 355.51 μmol, 1 eq) in THF (2 mL) was added t-BuONa (34 mg, 355.51 μmol, 1 eq) and the mixture was stirred at 25° C. for 0.5 hour. Then 4-(5-fluoro-2-isocyanato-3-isopropylphenyl)picolinonitrile (intermediate A37) (0.1 g, 355.51 μmol, 1 eq) was added and the resulting mixture was heated to 70° C. and stirred for 0.1 hour. The mixture was concentrated in vacuo. The residue was purified by prep-HPLC (column: Waters Xbridge C18, 150 mm*50 mm*10 μm; mobile phase: [A: water (0.05% NH₃.H₂O); B: MeCN]; B %: 15%-45%, 11.5 min) to give the title compound (0.038 g, 23% yield, 99% purity on LCMS) as a white solid.

¹H NMR (DMSO-d₆): δ 10.59 (br s, 1H), 8.77 (d, 1H), 8.12 (s, 1H), 7.80 (dd, 1H), 7.30-7.10 (m, 7H), 4.30 (s, 2H), 3.24-3.20 (m, 1H) and 1.20 (d, 6H).

LCMS: m/z 453.3 (M+H)⁺ (ES⁺).

### Example 128: N-((4-Fluoro-2-isopropyl-6-(2-methoxypyridin-4-yl)phenyl) carbamoyl)-1-phenylmethanesulfonamide

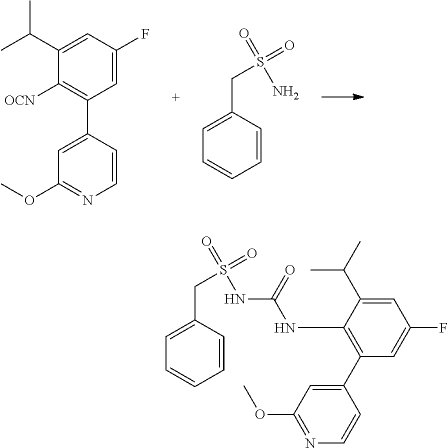

To a solution of phenylmethanesulfonamide (60 mg, 349.28 μmol, 1 eq) in THF (2 mL) was added t-BuONa (34 mg, 349.28 μmol, 1 eq) and the mixture was stirred at 25° C. for 0.5 hour. Then 4-(5-fluoro-2-isocyanato-3-isopropylphenyl)-2-methoxypyridine (intermediate A38) (0.1 g, 349.28 μmol, 1 eq) was added and the resulting mixture was heated to 70° C. and stirred for 0.1 hour. The mixture was concentrated in vacuo. The residue was purified by prep-HPLC (column: Waters Xbridge C18, 150 mm*50 mm*10 μm; mobile phase: [A: water (0.05% NH₃.H₂O); B: MeCN]; B %: 10%-40%, 11.5 min) to give the title compound (0.04 g, 25% yield, 99% purity on LCMS) as a white solid.

¹H NMR (DMSO-d₆): δ 8.15 (d, 1H), 7.52 (br s, 1H), 7.34-7.11 (m, 6H), 7.10-6.95 (m, 2H), 6.87 (s, 1H), 4.27 (s, 2H), 3.85 (s, 3H), 3.25-3.19 (m, 1H) and 1.18 (d, 6H).

LCMS: m/z 458.3 (M+H)⁺ (ES⁺).

### Example 129: N-((5-(2-Methoxypyridin-4-yl)-2,3-dihydro-1H-inden-4-yl) carbamoyl)-1-phenylmethanesulfonamide

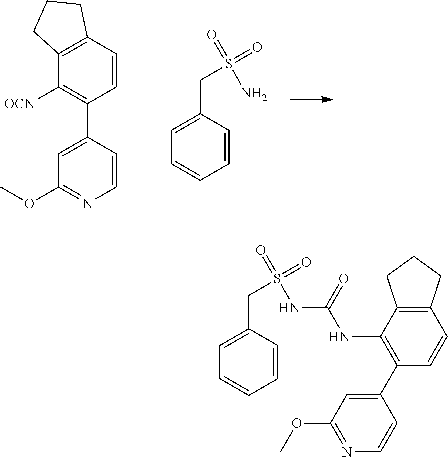

To a solution of phenylmethanesulfonamide (64 mg, 375.52 μmol, 1 eq) in THF (2 mL) was added t-BuONa (36 mg, 375.52 μmol, 1 eq) and the mixture was stirred at 25° C. for 0.5 hour. Then 4-(4-isocyanato-2,3-dihydro-1H-inden-5-yl)-2-methoxypyridine (intermediate A39) (0.1 g, 375.52 μmol, 1 eq) was added and the resulting mixture was heated to 70° C. and stirred for 0.1 hour. The mixture was concentrated in vacua.

The residue was purified by prep-HPLC (column: Waters Xbridge C18, 150 mm*50 mm*10 μm; mobile phase: [A: water (0.05% NH₃.H₂O); B: MeCN]; B %: 8%-38%, 11.5 min) to give the title compound (90.80 mg, 55% yield, 99% purity on LCMS) as a white solid.

¹H NMR (DMSO-d₆): δ 8.14 (d, 1H), 7.50 (br s, 1H), 7.32-7.30 (m, 3H), 7.25-7.24 (m, 2H), 7.17 (d, 1H), 7.09 (d, 1H), 6.97 (dd, 1H), 6.80 (s, 1H), 4.37 (s, 2H), 3.87 (s, 3H), 2.94 (t, 2H), 2.85 (t, 2H) and 2.09-1.97 (m, 2H).

LCMS: m/z 438.2 (M+H)⁺ (ES⁺).

### Example 130: N-((7-Fluoro-5-(pyridin-4-yl)-2,3-dihydro-1H-inden-4-yl) carbamoyl)-1-phenylmethanesulfonamide

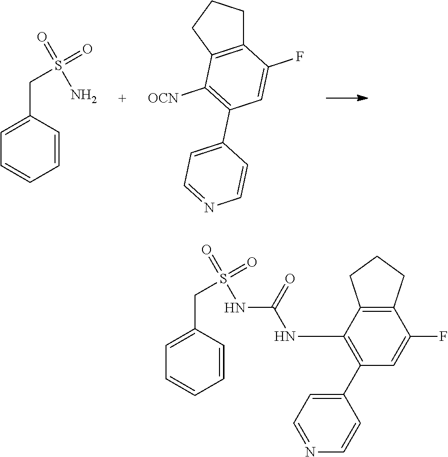

A mixture of phenylmethanesulfonamide (70 mg, 408.84 μmol, 1 eq) and t-BuONa (39 mg, 408.84 μmol, 1 eq) in THF (2 mL) was stirred at 25° C. for 10 minutes. Then 4-(7-fluoro-4-isocyanato-2,3-dihydro-1H-inden-5-yl)pyridine (intermediate A40) (104 mg, 408.84 μmol, 1 eq) was added. The mixture was stirred at 70° C. for 10 minutes. The reaction mixture was concentrated in vacua. The residue was purified by prep-HPLC (column: Xtimate C18, 250 mm*50 mm*10 μm; mobile phase: [A: water (0.05% ammonium hydroxide v/v); B: MeCN]; B %: 5%-35%, 10 min) to give the title compound (16.61 mg, 10% yield, 100% purity on LCMS) as a white solid.

¹H NMR (DMSO-d₆): δ 8.54 (d, 2H), 7.41 (d, 2H), 7.26-7.22 (m, 4H), 7.18-7.02 (m, 2H), 6.95 (d, 1H), 4.21 (s, 2H), 2.96 (t, 2H), 2.89 (t, 2H) and 2.12-2.03 (m, 2H).

LCMS: m/z 426.2 (M+H)⁺ (ES⁺).

### Example 131: N-((2-(2-Cyanopyridin-4-yl)-4-fluoro-6-isopropylphenyl) carbamoyl)-2-methylpropane-1-sulfonamide

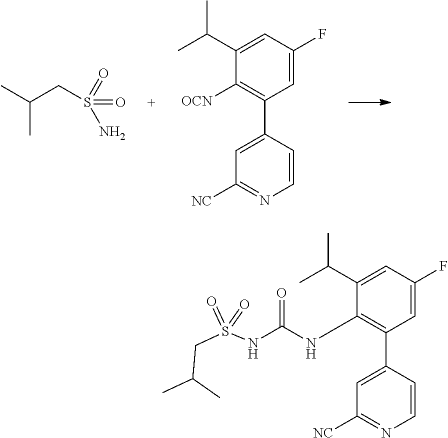

To a solution of 2-methylpropane-1-sulfonamide (49 mg, 355.51 μmol, 1 eq) (intermediate P26) in THF (2 mL) were added t-BuONa (34 mg, 355.51 μmol, 1 eq) and 4-(5-fluoro-2-isocyanato-3-isopropylphenyl)picolinonitrile (intermediate A37) (100 mg, 355.51 μmol, 1 eq). The reaction mixture was stirred at 20° C. for 20 minutes and then concentrated in vacuo. The residue was purified by prep-HPLC (column: Phenomenex Gemini C18, 150 mm*25 mm*5 μm, mobile phase: [A: water (0.05% ammonium hydroxide v/v); B: MeCN], B %: 3%-33%, 12.0 min) to give the title compound (48.16 mg, 32%) as a white solid.

¹H NMR (DMSO-d₆): δ 8.72 (d, 1H), 8.07 (s, 1H), 7.77 (s, 1H), 7.67 (s, 1H), 7.21 (d, 1H), 7.11 (d, 1H), 3.26-3.23 (m, 1H), 2.67-2.63 (m, 2H), 1.77-1.66 (m, 1H), 1.15 (d, 6H) and 0.84 (d, 6H).

LCMS: m/z 419.2 (M+H)⁺ (ES⁺).

### Example 132: N-((4-Fluoro-2-isopropyl-6-(2-methoxypyridin-4-yl)phenyl) carbamoyl)-2-methylpropane-1-sulfonamide

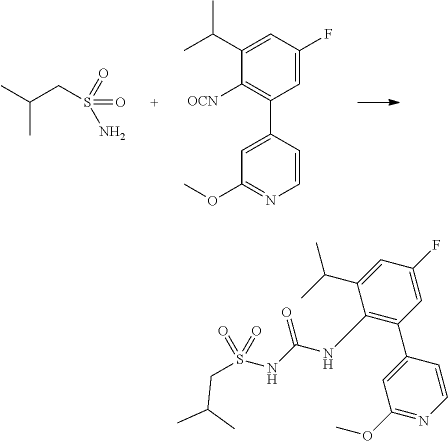

To a solution of 2-methylpropane-1-sulfonamide (intermediate P26) (48 mg, 349.28 μmol, 1 eq) in THF (2 mL) were added t-BuONa (34 mg, 349.28 μmol, 1 eq) and 4-(5-fluoro-2-isocyanato-3-isopropylphenyl)-2-methoxypyridine (intermediate A38) (100 mg, 349.28 μmol, 1 eq). The reaction mixture was stirred at 25° C. for 10 minutes and then was concentrated in vacua. The residue was purified by prep-HPLC (column: Phenomenex Gemini C18, 150 mm*25 mm*5 μm, mobile phase: [A: water (0.05% ammonium hydroxide v/v); B: MeCN], B %: 15%-45%, 11.5 min) to give the title compound (101.64 mg, 69% yield, 100% purity on LCMS) as a white solid. ¹H NMR (DMSO-d₆): δ 8.17 (d, 1H), 7.91 (s, 1H), 7.27-7.24 (m, 1H), 7.06 (dd, 1H), 6.99 (d, 1H), 6.82 (s, 1H), 3.87 (s, 3H), 3.16-3.09 (m, 1H), 3.00 (d, 2H), 1.91-1.81 (m, 1H), 1.16 (d, 6H) and 0.91 (d, 6H).

LCMS: m/z 424.2 (M+H)⁺ (ES^{+).}

### Example 133: N-((5-(2-Methoxypyridin-4-yl)-2,3-dihydro-1H-inden-4-yl) carbamoyl)-2-methylpropane-1-sulfonamide

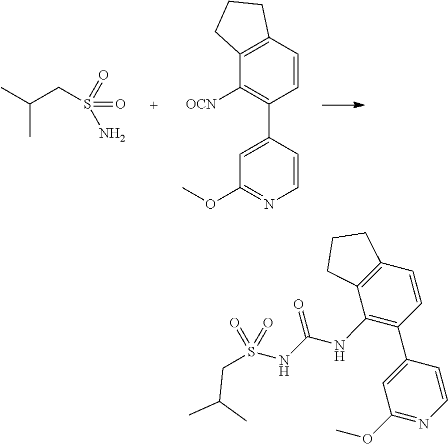

To a solution of 2-methylpropane-1-sulfonamide (intermediate P26) (55 mg, 401.36 μmol, 1 eq) in THF (2 mL) were added t-BuONa (39 mg, 401.36 μmol, 1 eq) and 4-(4-isocyanato-2,3-dihydro-1H-inden-5-yl)-2-methoxypyridine (intermediate A39) (167 mg, 401.36 μmol, 1 eq). The reaction mixture was stirred at 25° C. for 20 minutes and then concentrated in vacuo. The residue was purified by prep-HPLC (column: Phenomenex Gemini C18, 150 mm*25 mm*10 μm, mobile phase: [A: water (10 mM NH₄HCO₃); B: MeCN], B %: 18%-48%, 10 min) to give the title compound (16.29 mg, 10%) as a white solid.

¹H NMR (DMSO-d₆): δ 8.15 (d, 1H), 7.93 (br s, 1H), 7.22 (d, 1H), 7.12 (d, 1H), 6.94-6.91 (m, 1H), 6.74 (s, 1H), 3.86 (s, 3H), 3.10 (d, 2H), 2.93 (t, 2H), 2.79 (t, 2H), 2.05-1.95 (m, 3H) and 0.95 (d, 6H).

LCMS: m/z 404.2 (M+H)⁺ (ES^{+).}

### Example 134: N-((7-Fluoro-5-(pyridin-4-yl)-2,3-dihydro-1H-inden-4-yl) carbamoyl)-2-methylpropane-1-sulfonamide

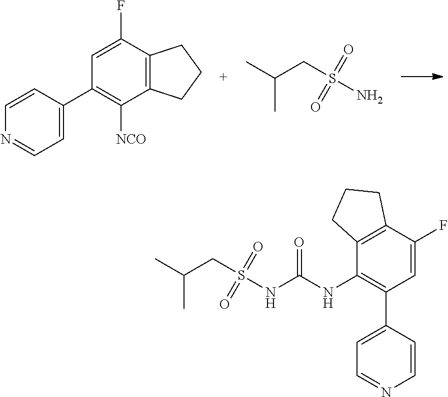

To a solution of 2-methylpropane-1-sulfonamide (intermediate P26) (54 mg, 393.30 μmol, 1 eq) in THF (2 mL) was added t-BuONa (38 mg, 393.30 μmol, 1 eq). Then the mixture was stirred at 25° C. for 10 minutes. A solution of 4-(7-fluoro-4-isocyanato-2,3-dihydro-1H-inden-5-yl)pyridine (intermediate A40) (100 mg, 393.30 μmol, 1 eq) in THF (2.5 mL) was added. The resulting mixture was stirred at 25° C. for 30 minutes and then concentrated in vacuo. The residue was purified by prep-HPLC (Column: Xtimate C18, 250 mm*50 mm*10 μm, mobile phase: [A: water (0.05% ammonium hydroxide v/v); B: MeCN], B %: 1%-31%, 10.0 min) to give the title compound (45.33 mg, 29% yield, 100% purity on LCMS) as a white solid.

¹H NMR (DMSO-d₆): δ 8.54 (d, 2H), 7.40 (d, 2H), 6.96 (d, 1H), 2.95 (t, 2H), 2.89-2.83 (m, 4H), 2.09-2.03 (m, 2H), 1.96-1.91 (m, 1H) and 0.93 (d, 6H).

LCMS: m/z 392.2 (M+H)⁺ (ES^{+).}

### Example 135: N-((2-(2-Cyanopyridin-4-yl)-4-fluoro-6-isopropylphenyl) carbamoyl)-2-phenylethanesulfonamide

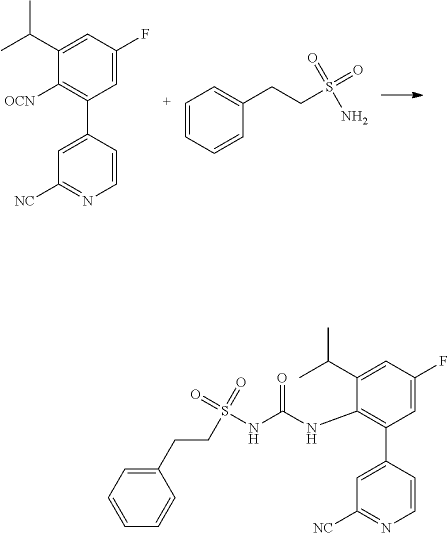

To a solution of 2-phenylethanesulfonamide (intermediate P27) (66 mg, 355.51 μmol, 1 eq) in THF (2 mL) was added t-BuONa (34 mg, 355.51 μmol, 1 eq) and the mixture was stirred at 25° C. for 0.5 hour. Then 4-(5-fluoro-2-isocyanato-3-isopropylphenyl)picolinonitrile (intermediate A37) (0.1 g, 355.51 μmol, 1 eq) was added and the resulting mixture was heated to 70° C. and stirred for 0.1 hour. The mixture was concentrated in vacuo. The residue was purified by prep-HPLC (column: Waters Xbridge C18, 150 mm*50 mm*10 μm; mobile phase: [A: water (0.05% NH₃.H₂O); B: MeCN]; B %: 12%-42%, 11.5 min) to give the title compound (0.07 g, 42% yield, 99% purity on LCMS) as a white solid.

¹H NMR (DMSO-d₆): δ 10.77 (br s, 1H), 8.67 (d, 1H), 8.11 (s, 1H), 7.92 (br s, 1H), 7.80 (d, 1H), 7.31-7.18 (m, 5H), 7.09 (d, 2H), 3.25-3.19 (m, 3H), 2.70-2.51 (m, 2H) and 1.17 (d, 6H).

LCMS: m/z 467.3 (M+H)⁺ (ES^{+).}

### Example 136: N-((4-Fluoro-2-isopropyl-6-(2-methoxypyridin-4-yl)phenyl) carbamoyl)-2-phenylethanesulfonamide

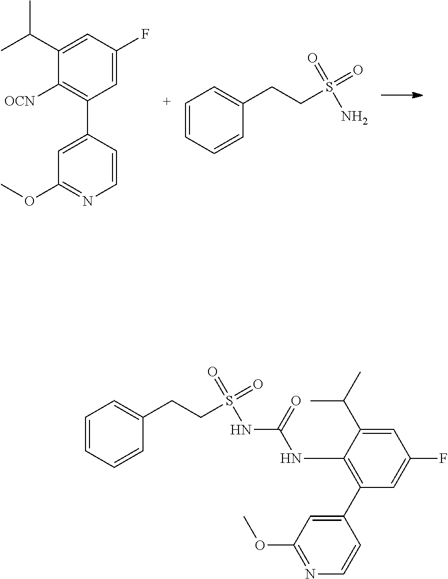

To a solution of 2-phenylethanesulfonamide (intermediate P27) (65 mg, 349.28 μmol, 1 eq) in THF (2 mL) was added t-BuONa (34 mg, 349.28 μmol, 1 eq) and the mixture was stirred at 25° C. for 0.5 hour. Then 4-(5-fluoro-2-isocyanato-3-isopropylphenyl)-2-methoxypyridine (intermediate A38) (0.1 g, 349.28 μmol, 1 eq) was added and the resulting mixture was heated to 70° C. and stirred for 0.1 hour. The mixture was concentrated in vacuo. The residue was purified by prep-HPLC (column: Phenomenex Gemini C18, 150 mm*25 mm*10 μm; mobile phase: [A: water (0.05% NH₃.H₂O); B: MeCN]; B %: 22%-52%, ii min) to give the title compound (0.0317 g, 19% yield, 99% purity on LCMS) as a white solid.

¹H NMR (DMSO-d₆): δ 8.10 (d, 1H), 8.00 (br s, 1H), 7.34-7.22 (m, 4H), 7.16-6.99 (m, 4H), 6.84 (s, 1H), 3.73 (s, 3H), 3.44-3.40 (m, 2H), 3.18-3.13 (m, 1H), 2.80-2.76 (m, 2H) and 1.16 (d, 6H).

LCMS: m/z 472.2 (M+H)⁺ (ES⁺).

### Example 137: N-((5-(2-Methoxypyridin-4-yl)-2,3-dihydro-1H-inden-4-yl) carbamoyl)-2-phenylethanesulfonamide

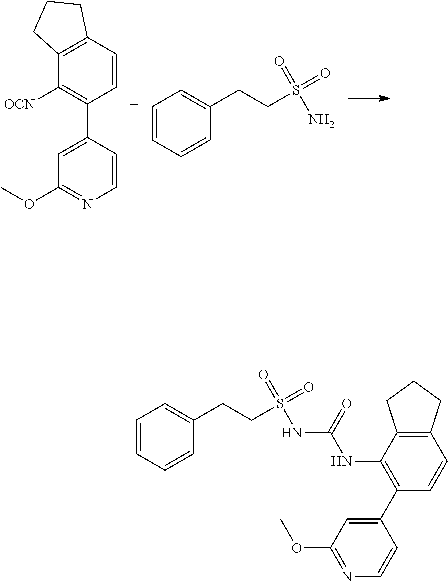

To a solution of 2-phenylethanesulfonamide (intermediate P27) (70 mg, 375.52 μmol, 1 eq) in THF (2 mL) was added t-BuONa (36 mg, 375.52 μmol, 1 eq) and the mixture was stirred at 25° C. for 0.5 hour. Then 4-(4-isocyanato-2,3-dihydro-1H-inden-5-yl)-2-methoxypyridine (intermediate A39) (0.1 g, 375.52 μmol, 1 eq) was added and the resulting mixture was heated to 70° C. and stirred for 0.1 hour. The mixture was concentrated in vacuo. The residue was purified by prep-HPLC (column: Phenomenex Gemini C18, 150 mm*25 mm*10 μm; mobile phase: [A: water (10 mM NH₄HCO₃); B: MeCN]; B %: 17%-47%, 11 min) to give the title compound (0.021 g, 12% yield, 100% purity on LCMS) as a white solid.

¹H NMR (DMSO-d₆): δ 8.07 (d, 1H), 7.50 (br s, 1H), 7.33-7.26 (m, 2H), 7.19-7.13 (m, 4H), 7.10-7.08 (m, 1H), 6.99 (d, 1H), 6.81 (s, 1H), 3.77 (s, 3H), 3.30-3.23 (m, 2H), 2.92 (t, 2H), 2.86-2.80 (m, 4H) and 2.07-1.98 (m, 2H).

LCMS: m/z 452.2 (M+H)⁺ (ES⁺).

### Example 138: N-((7-Fluoro-5-(pyridin-4-yl)-2,3-dihydro-1H-inden-4-yl) carbamoyl)-2-phenylethanesulfonamide

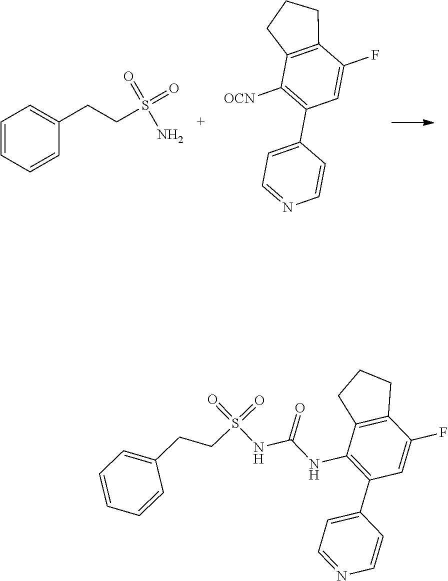

A mixture of 2-phenylethanesulfonamide (intermediate P27) (75 mg, 404.87 μmol, 1 eq) and t-BuONa (39 mg, 404.87 μmol, 1 eq) in THF (2 mL) was stirred at 25° C. for 10 minutes. Then 4-(7-fluoro-4-isocyanato-2,3-dihydro-1H-inden-5-yl)pyridine (intermediate A40) (103 mg, 404.87 μmol, 1 eq) was added. The resulting mixture was stirred at 25° C. for 10 minutes, then warmed to 70° C. and stirred for 10 minutes. The reaction mixture was concentrated in vacua. The residue was purified by prep-HPLC (column: Xtimate C18, 250 mm*50 mm*10 μm; mobile phase: [A: water (0.05% ammonium hydroxide v/v); B: MeCN]; B %: 5%-35%, 10 min) to give the title compound (15.1 mg, 8% yield, 100% purity on LCMS) as a white solid.

¹H NMR (DMSO-d₆): δ 8.53 (d, 2H), 7.63 (br s, 1H), 7.42 (d, 2H), 7.31 (t, 2H), 7.23-7.16 (m, 3H), 7.00 (d, 1H), 3.39-3.35 (m, 2H), 2.99 (t, 2H), 2.90-2.82 (m, 4H) and 2.10-2.06 (m, 2H).

LCMS: m/z 440.2 (M+H)⁺ (ES⁺).

### Example 139: N-((2-(2-Cyanopyridin-4-yl)-4-fluoro-6-isopropylphenyl) carbamoyl)-1-phenylethanesulfonamide

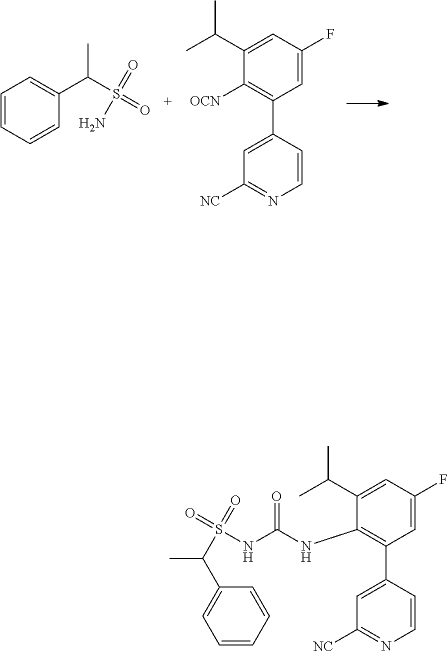

To a solution of 1-phenylethanesulfonamide (intermediate P28) (50 mg, 269.92 μmol, 1 eq) in THF (2 mL) was added t-BuONa (26 mg, 269.92 μmol, 1 eq). After stirring at 20° C. for 10 minutes, 4-(5-fluoro-2-isocyanato-3-isopropylphenyl)picolinonitrile (intermediate A37) (76 mg, 269.92 μmol, 1 eq) was added. The reaction mixture was stirred at 20° C. for 20 minutes and then concentrated in vacuo. The residue was purified by prep-HPLC (column: Phenomenex Gemini c18, 150 mm*25 mm*10 μm; mobile phase: [A: water (10 mM NH₄HCO₃); B: MeCN]; B %: 22%-52%, 12 min) to give the title compound (14.74 mg, ii % yield, 98% purity on LCMS) as a white solid.

¹H NMR (DMSO-d₆): δ 10.53 (br s, 1H), 8.77 (d, 1H), 8.10 (s, 1H), 7.97-7.93 (m, 1H), 7.77 (d, 1H), 7.32-7.24 (m, 4H), 7.23-7.19 (m, 3H), 4.57-4.54 (m, 1H), 3.15-3.12 (m, 1H), 1.46-1.40 (m, 3H) and 1.20-1.08 (m, 6H).

LCMS: m/z 467.2 (M+H)⁺ (ES⁺).

### Example 140: N-((4-Fluoro-2-isopropyl-6-(2-methoxypyridin-4-yl)phenyl) carbamoyl)-1-phenylethanesulfonamide

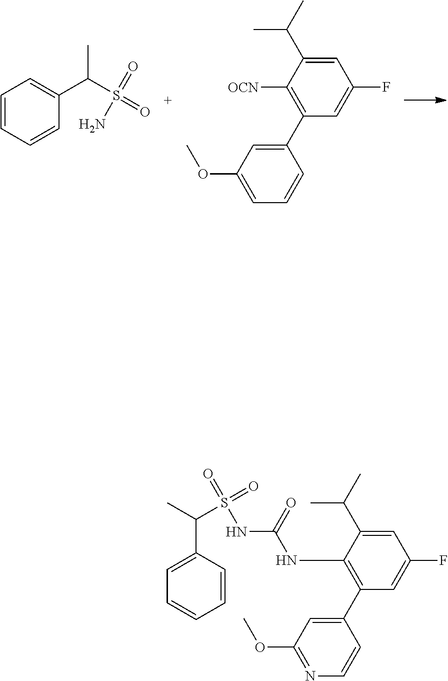

To a solution of 1-phenylethanesulfonamide (intermediate P28) (50 mg, 269.92 μmol, 1 eq) in THF (2 mL) was added t-BuONa (26 mg, 269.92 μmol, 1 eq). The mixture was stirred at 20° C. for 10 minutes. Then 4-(5-fluoro-2-isocyanato-3-isopropylphenyl)-2-methoxypyridine (intermediate A38) (77 mg, 269.92 μmol, 1 eq) was added. The reaction mixture was stirred at 20° C. for 20 minutes and then concentrated in vacua. The residue was purified by prep-HPLC (column: Xtimate C18, 150 mm*25 mm*5 μm; mobile phase: [A: water (0.05% ammonium hydroxide v/v); B: MeCN]; B %: 10%-40%, 12 min) to give the title compound (12.98 mg, 10% yield, 100% purity on LCMS) as a white solid.

¹H NMR (DMSO-d₆): δ 10.40 (br s, 1H), 8.15 (d, 1H), 7.70 (br s, 1H), 7.32-7.20 (m, 6H), 7.05-7.00 (m, 2H), 6.85 (s, 1H), 4.60-4.56 (m, 1H), 3.86 (s, 3H), 3.16-3.11 (m, 1H), 1.45 (d, 3H) and 1.18 (dd, 6H).

LCMS: m/z 472.2 (M+H)⁺ (ES⁺).

### Example 141: N-((5-(2-Methoxypyridin-4-yl)-2,3-dihydro-1H-inden-4-yl) carbamoyl)-1-phenylethanesulfonamide

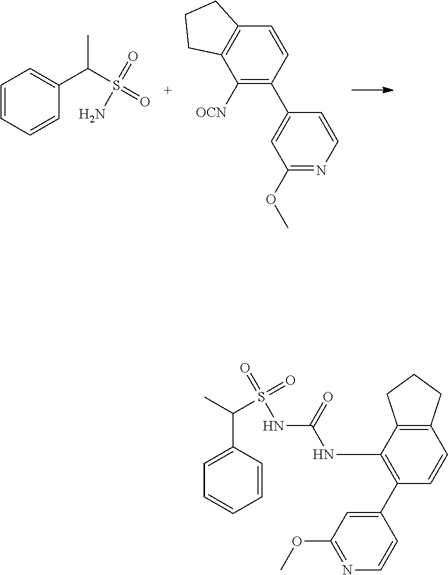

To a solution of 1-phenylethanesulfonamide (intermediate P28) (50 mg, 269.92 μmol, 1 eq) in THF (2 mL) was added t-BuONa (26 mg, 269.92 μmol, 1 eq). The mixture was stirred at 20° C. for 10 minutes. Then 4-(4-isocyanato-2,3-dihydro-1H-inden-5-yl)-2-methoxypyridine (intermediate A39) (72 mg, 269.92 μmol, 1 eq) was added and then the resulting mixture was stirred at 20° C. for 20 minutes. The reaction mixture was concentrated in vacua. The residue was purified by prep-HPLC (column: Xtimate C18, 150 mm*25 mm*5 μm; mobile phase: [A: water (0.05% ammonium hydroxide v/v); B: MeCN]; B %: 5%-35%, 12 min) to give the title compound (34.56 mg, 28% yield, 99.8% purity on LCMS) as a white solid.

¹H NMR (DMSO-d₆): δ 8.12 (d, 1H), 7.60 (br s, 1H), 7.33-7.30 (m, 5H), 7.19 (d, 1H), 7.09 (d, 1H), 6.94-6.92 (m, 1H), 6.77 (s, 1H), 4.69-4.66 (m, 1H), 3.86 (s, 3H), 2.93 (t, 2H), 2.81 (t, 2H), 2.07-2.01 (m, 2H) and 1.54 (d, 3H).

LCMS: m/z 452.2 (M+H)⁺ (ES⁺).

### Example 142: N-((7-Fluoro-5-(pyridin-4-yl)-2,3-dihydro-1H-inden-4-yl) carbamoyl)-1-phenylethanesulfonamide

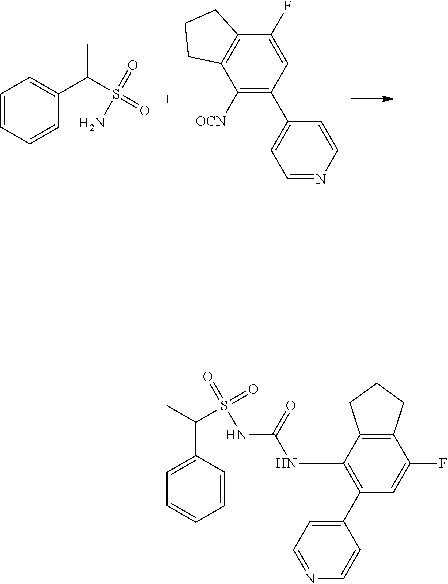

To a solution of 1-phenylethanesulfonamide (intermediate P28) (75 mg, 404.87 μmol, 1 eq) in THF (2 mL) was added t-BuONa (39 mg, 404.87 μmol, 1 eq). Then the reaction mixture was stirred at 20° C. for 10 minutes. A solution of 4-(7-fluoro-4-isocyanato-2,3-dihydro-1H-inden-5-yl)pyridine (intermediate A40) (103 mg, 404.87 μmol, 1 eq) in THF (2 mL) was added. The resulting mixture was stirred at 20° C. for 20 minutes and then concentrated in vacua. The residue was purified by prep-HPLC (column: Phenomenex Gemini C18, 150 mm*25 mm*10 μm; mobile phase: [A: water (10 mM NH₄HCO₃); B: MeCN]; B %: 13%-43%, 10 min) to give the title compound (63.22 mg, 35% yield, 99% purity on LCMS) as a light red solid.

¹H NMR (DMSO-d₆): δ 8.57 (d, 2H), 7.69 (br s, 1H), 7.37-7.30 (m, 7H), 7.02 (d, 1H), 4.75-4.67 (m, 1H), 2.98 (t, 2H), 2.84 (t, 2H), 2.14-2.08 (m, 2H) and 1.55 (d, 3H).

LCMS: m/z 440.2 (M+H)⁺ (ES^{+).}

### Example 143: N-((5-(2-Methoxypyridin-4-yl)-2,3-dihydro-1H-inden-4-yl) carbamoyl)methanesulfonamide

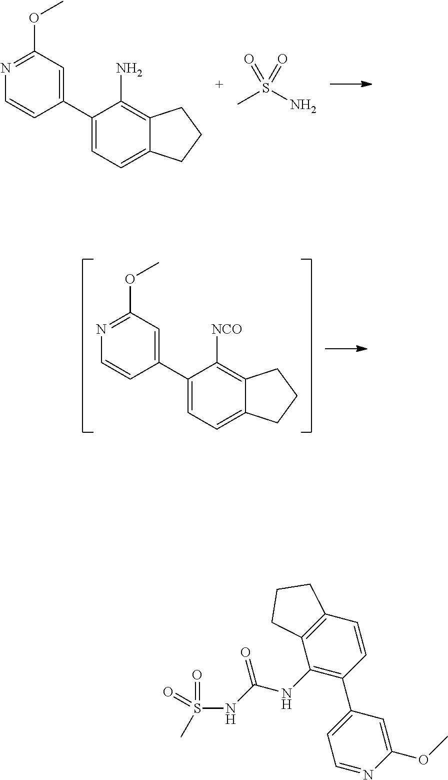

5-(2-Methoxypyridin-4-yl)-2,3-dihydro-1H-inden-4-amine (326 mg, 1.36 mmol) (Intermediate A35) was dissolved in THF (5 mL). Triethylamine (208 μl, 1.49 mmol) was added, followed by a solution of bis(trichloromethyl) carbonate (382 mg, 1.29 mmol) in THF (2 mL). The thick reaction mixture was stirred at room temperature for 1 hour. The solvent was removed in vacua and the solid formed was dried under high vacuum for 1 hour. The solid, 4-(4-isocyanato-2,3-dihydro-1H-inden-5-yl)-2-methoxypyridine, was suspended in THF (8 mL) of which 2 mL were used later. Methanesulfonamide (30 mg, 0.315 mmol) was suspended in THF (2 mL), sodium tert-butoxide (2 M in THF) (175 μl, 0.351 mmol) was added, and the mixture was stirred for 30 minutes at room temperature. Then a solution of 4-(4-isocyanato-2,3-dihydro-1H-inden-5-yl)-2-methoxypyridine (78 mg, 0.292 mmol) in THF (2 mL), prepared earlier, was added and the mixture was stirred overnight at room temperature. The THF was removed in vacuo and the residue was dissolved in DMSO (2 mL) and then purified by basic prep HPLC to afford the title compound (23.5 mg, 21%) as a colourless solid.

¹H NMR (DMSO-d₆): δ 8.17 (d, J=5.3 Hz, 1H), 7.86 (s, 1H), 7.22 (d, J=7.9 Hz, 1H), 7.14 (d, J=7.7 Hz, 1H), 6.95 (dd, J=5.3, 1.3 Hz, 1H), 6.77 (s, 1H), 3.88 (s, 3H), 3.01 (s, 3H), 2.94 (t, J=7.4 Hz, 2H), 2.82 (t, J=7.4 Hz, 2H), 2.04 (p, J=7.5 Hz, 2H), NH not observed.

LCMS; m/z 362.2 (M+H)⁺ (ES⁺); 360.0 (M−H)⁻ (ES⁻).

### Example 144: 1-Cyclopropyl-N-((6-(2-methoxypyridin-4-yl)-2,3-dihydro-1H-inden-5-yl)carbamoyl)-1H-pyrazole-3-sulfonamide

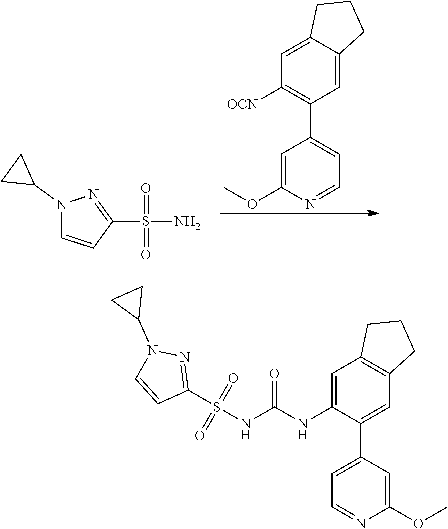

To a solution of 1-cyclopropyl-1H-pyrazole-3-sulfonamide (Intermediate P29) (50 mg, 267.07 μmol, 0.7 eq) in THF (1.5 mL) was added t-BuONa (36 mg, 375.52 μmol, 1 eq) and 4-(6-isocyanato-2,3-dihydro-1H-inden-5-yl)-2-methoxypyridine (Intermediate A45) (100 mg, 375.52 μmol, 1 eq). The mixture was stirred at 25° C. for 0.5 hour. Most of the solvent was concentrated to give crude product. The residue was purified by prep-HPLC (column: Xtimate C18, 150 mm*25 mm*5 μm; mobile phase: [A: water (0.05% ammonium hydroxide v/v); B: MeCN]; B %: 9%-39%, 8 min) to give the title compound (22.39 mg, 13% yield, 98% purity on LCMS) as a white solid.

¹H NMR (DMSO-d₆): δ 8.19 (d, 1H), 7.80-7.74 (m, 2H), 7.24 (br s, 1H), 7.01 (s, 1H), 6.91 (d, 1H), 6.72 (s, 1H), 6.42 (s, 1H), 3.89 (s, 3H), 3.76-3.73 (m, 1H), 2.84-2.78 (m, 4H), 2.04-1.98 (m, 2H), and 1.03-0.95 (d, 4H).

LCMS: m/z 454.3 (M+H)⁺ (ES⁺).

### Example 145: 1-Cyclopropyl-N-((5-(2-methoxypyridin-4-yl)-2,3-dihydro-1H-inden-4-yl)carbamoyl)-1H-pyrazole-4-sulfonamide

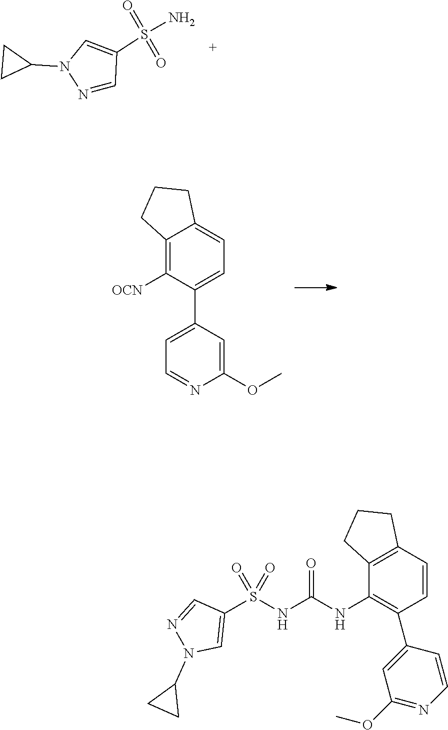

To a solution of 4-(4-isocyanato-2,3-dihydro-1H-inden-5-yl)-2-methoxypyridine (intermediate A39) (71 mg, 267.07 μmol, 1 eq) in THF (1 mL) was added t-BuONa (26 mg, 267.07 μmol, 1 eq) and 1-cyclopropyl-1H-pyrazole-4-sulfonamide (intermediate P30) (50 mg, 267.07 μmol, 1 eq). The mixture was stirred at 25° C. for 20 minutes. Most of the solvent was evaporated to give crude product. The crude product was purified by prep-HPLC (column: Waters Xbridge C18, 150 mm*25 mm*5 μm; mobile phase: [A: water (0.05% ammonium hydroxide v/v); B: MeCN]; B %: 12%-42%, 10 min) to give the title compound (12.82 mg, 11% yield, 100% purity on LCMS) as a white solid.

¹H NMR (DMSO-d₆): δ 8.16 (s, 1H), 8.04-8.03 (d, 1H), 7.69 (s, 1H), 7.63 (s, 1H), 7.18-7.16 (d, 1H), 7.09-7.07 (d, 1H), 6.82-6.80 (d, 1H), 6.68 (s, 1H), 3.87 (s, 3H), 3.85-3.78 (m, 1H), 2.92-2.89 (m, 2H), 2.68-2.64 (m, 2H), 2.011.94 (m, 2H), 1.06-1.03 (m, 2H) and 1.01-0.96 (m, 2H).

LCMS: m/z 454.4 (M+H)⁺ (ES⁺).

### Example 146: N-((2-(2-Cyanopyridin-4-yl)-4-fluoro-6-isopropylphenyl) carbamoyl)-3-(diethylamino)propane-1-sulfonamide

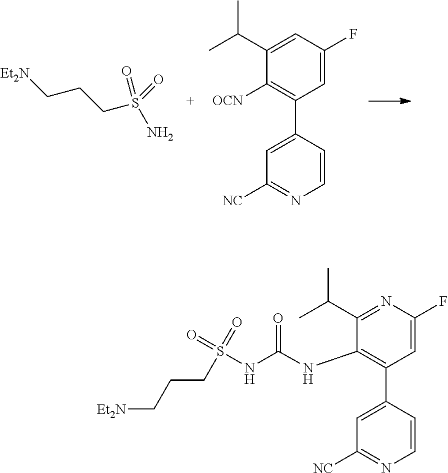

To a solution of 3-(diethylamino)propane-1-sulfonamide (Intermediate P32) (80 mg, 411.75 μmol, 1 eq) in THF (1 mL) was added t-BuONa (40 mg, 411.75 μmol, 1 eq) and the mixture was stirred at 25° C. for 10 minutes. Then 4-(5-fluoro-2-isocyanato-3-isopropylphenyl)picolinonitrile (Intermediate A37) (116 mg, 411.75 μmol, 1 eq) was added. The resulting mixture was stirred at 70° C. for 10 minutes. The mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 12%-42%, 11.5 min) to give the title compound (105.29 mg, 55% yield, 100% purity on LCMS) as a white solid.

¹H NMR (DMSO-d₆): δ 8.75 (d, 1H), 8.08 (s, 1H), 7.79-7.73 (m, 2H), 7.23 (d, 1H), 7.13 (d, 1H), 3.09-3.06 (m, 1H), 3.03-2.88 (m, 8H), 1.75-1.72 (m, 2H), 1.16 (d, 6H) and 1.09 (t, 6H).

LCMS: m/z 476.3 (M+H)⁺ (ES⁺).

### Example 147: 3-(Diethylamino)-N-((4-fluoro-2-isopropyl-6-(2-methoxypyridin-4-yl)phenyl)carbamoyl)propane-1-sulfonamide

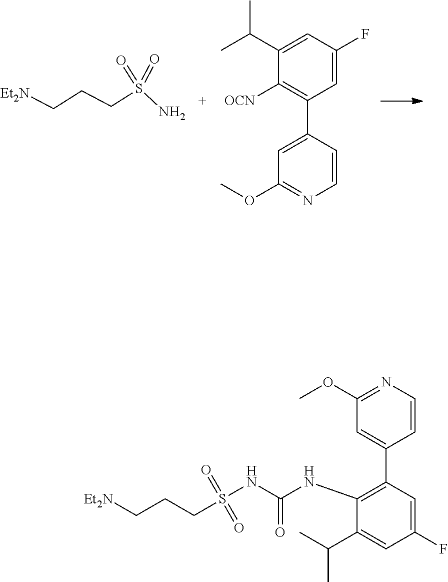

To a solution of 3-(diethylamino)propane-1-sulfonamide (Intermediate P32) (80 mg, 411.75 μmol, 1 eq) in THF (1 mL) was added t-BuONa (40 mg, 411.75 μmol, 1 eq) and the mixture was stirred at 25° C. for 10 minutes. Then 4-(5-fluoro-2-isocyanato-3-isopropylphenyl)-2-methoxypyridine (Intermediate A38) (118 mg, 411.75 μmol, 1 eq) was added. The resulting mixture was stirred at 70° C. for 10 minutes. The mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 18%-48%, 11.5 min) to give the title compound (59.65 mg, 30% yield, 100% purity on LCMS) as a white solid.

¹H NMR (DMSO-d₆): δ 8.15 (d, 1H), 7.64 (s, 1H), 7.19 (d, 1H), 7.09-6.95 (m, 2H), 6.85 (s, 1H), 3.87 (s, 3H), 3.23-3.20 (m, 1H), 3.04-2.75 (m, 8H), 1.77-1.72 (m, 2H), 1.16 (d, 6H) and 1.09-1.04 (m, 6H).

LCMS: m/z 481.3 (M+H)⁺ (ES⁺).

### Example 148: 3-(Diethylamino)-N-((5-(2-methoxypyridin-4-yl)-2,3-dihydro-1H-inden-4-yl)carbamoyl)propane-1-sulfonamide

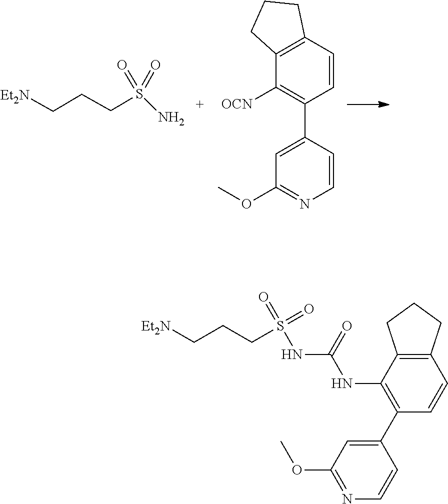

To a solution of 3-(diethylamino)propane-1-sulfonamide (Intermediate P32) (80 mg, 411.75 μmol, 1 eq) in THF (1 mL) was added t-BuONa (40 mg, 411.75 μmol, 1 eq) and the mixture was stirred at 25° C. for 10 minutes. Then 4-(4-isocyanato-2,3-dihydro-1H-inden-5-yl)-2-methoxypyridine (Intermediate A39) (171 mg, 411.75 μmol, purity: 64% on LCMS, 1 eq) was added. The resulting mixture was stirred at 70° C. for 10 minutes. The mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 15%-45%, 11.5 min) to give the title compound (53.15 mg, 28% yield, 100% purity on LCMS) as a pink solid.

¹H NMR (DMSO-d₆): δ 8.13 (d, 1H), 7.64 (br s, 1H), 7.15 (d, 1H), 7.09 (d, 1H), 6.97 (dd, 1H), 6.78 (s, 1H), 3.86 (s, 3H), 3.08 (t, 2H), 2.91 (t, 2H), 2.85-2.76 (m, 8H), 2.03-2.00 (m, 2H), 1.82-1.78 (m, 2H) and 1.05 (t, 6H).

LCMS: m/z 461.3 (M+H)⁺ (ES⁺).

### Example 149: 3-(Diethylamino)-N-((7-fluoro-5-(pyridin-4-yl)-2,3-dihydro-1H-inden-4-yl)carbamoyl)propane-1-sulfonamide

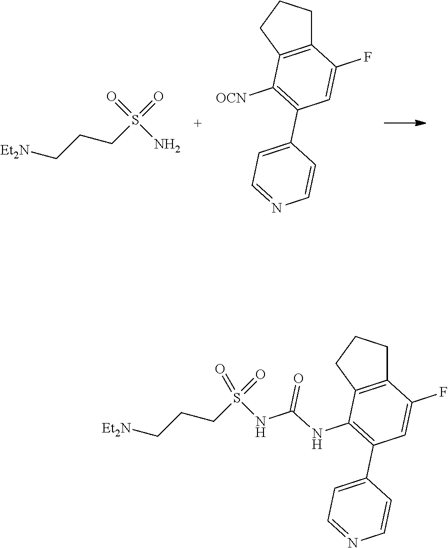

A mixture of 3-(diethylamino)propane-1-sulfonamide (Intermediate P32) (60 mg, 308.81 μmol, 1 eq) and t-BuONa (30 mg, 308.81 μmol, 1 eq) in THF (2 mL) was stirred at 25° C. for 10 minutes. Then 4-(7-fluoro-4-isocyanato-2,3-dihydro-1H-inden-5-yl)pyridine (Intermediate A40) (78 mg, 308.81 μmol, 1 eq) was added. The resulting mixture was stirred at 25° C. for 10 minutes. The reaction mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 10%-40%, 10 minutes) to give the title compound (18.1 mg, 13% yield, 100% purity on LCMS) as a white solid.

¹H NMR (DMSO-d₆): δ 8.58-8.56 (m, 2H), 7.61 (br s, 1H), 7.41 (d, 2H), 6.99 (d, 1H), 3.03 (t, 2H), 2.96 (t, 2H), 2.90-2.78 (m, 8H), 2.11-2.04 (m, 2H), 1.82-1.75 (m, 2H) and 1.07 (t, 6H).

LCMS: m/z 449.2 (M+H)⁺ (ES⁺).

### Example 150: 3-(Benzyl(ethyl)amino)-N-((4-fluoro-2-isopropyl-6-(2-methoxypyridin-4-yl)phenyl)carbamoyl)propane-1-sulfonamide

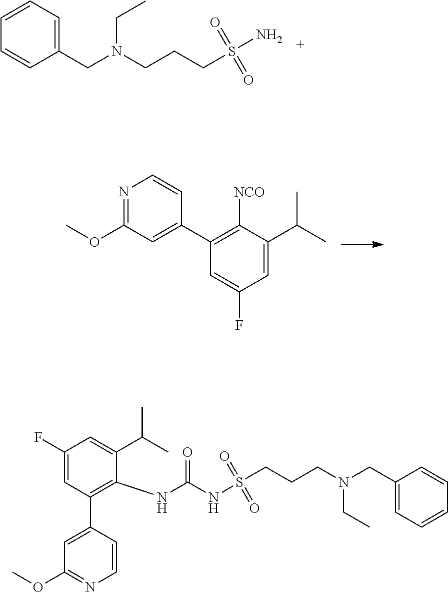

To a solution of 3-(benzyl(ethyl)amino)propane-1-sulfonamide (Intermediate P33) (90 mg, 351.06 μmol, 1 eq) in THF (1 mL) was added t-BuONa (34 mg, 351.06 μmol, 1 eq) and the mixture was stirred at 25° C. for 10 minutes. Then 4-(5-fluoro-2-isocyanato-3-isopropylphenyl)-2-methoxypyridine (Intermediate A38) (101 mg, 351.06 μmol, 1 eq) was added. The resulting mixture was stirred at 70° C. for 10 minutes. The mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 20%-50%, 11.5 min) to give the title compound (66.21 mg, 35% yield, 100% purity on LCMS) as a white solid.

¹H NMR (DMSO-d₆): δ 8.13 (d, 1H), 7.72 (s, 1H), 7.32-7.20 (m, 6H), 7.06-7.01 (m, 2H), 6.83 (s, 1H), 3.85 (s, 3H), 3.53 (s, 2H), 3.19-3.15 (m, 1H), 3.04-3.01 (m, 2H), 2.44-2.40 (m, 4H), 1.68-1.64 (m, 2H), 1.15 (d, 6H) and 0.96 (t, 3H).

LCMS: m/z 543.4 (M+H)⁺ (ES⁺).

### Example 151: 3-(Benzyl(ethyl)amino)-N-((2-(2-cyanopyridin-4-yl)-4-fluoro-6-isopropylphenyl)carbamoyl)propane-1-sulfonamide

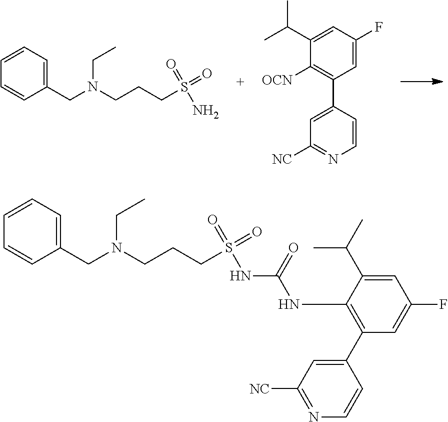

To a solution of 3-(benzyl(ethyl)amino)propane-1-sulfonamide (Intermediate P33) (100 mg, 390.07 μmol, 1 eq) in THF (1 mL) was added t-BuONa (37 mg, 390.07 μmol, 1 eq) and the mixture was stirred at 25° C. for 10 minutes. Then 4-(5-fluoro-2-isocyanato-3-isopropylphenyl)picolinonitrile (Intermediate A37) (no mg, 390.07 μmol, 1 eq) was added. The resulting mixture was stirred at 70° C. for 10 minutes. The mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 28%-58%, 11.5 min) to give the title compound (37.69 mg, 18% yield, 100% purity on LCMS) as a white solid.

¹H NMR (DMSO-d₆): δ 10.49 (br s, 1H), 8.76 (d, 1H), 8.14 (s, 1H), 8.09 (s, 1H), 7.76 (dd, 1H), 7.34-7.20 (m, 7H), 3.74 (s, 2H), 3.18-3.09 (m, 3H), 2.47-2.42 (m, 4H), 1.65-1.62 (m, 2H), 1.17 (d, 6H) and 0.96 (t, 3H).

LCMS: m/z 538.4 (M+H)⁺ (ES⁺).

### Example 152: 3-(Benzyl(ethyl)amino)-N-((5-(2-methoxypyridin-4-yl)-2,3-dihydro-1H-inden-4-yl)carbamoyl)propane-1-sulfonamide

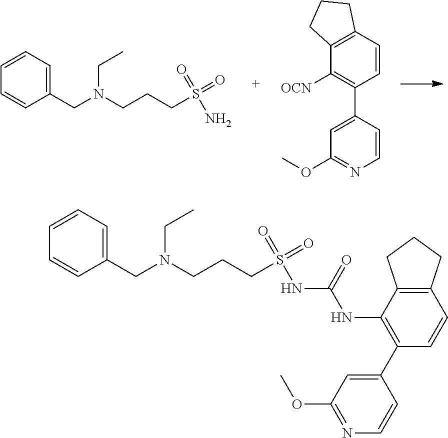

To a solution of 3-(benzyl(ethyl)amino)propane-1-sulfonamide (Intermediate P33) (100 mg, 390.07 μmol, 1 eq) in THF (1 mL) was added t-BuONa (37 mg, 390.07 μmol, 1 eq) and the mixture was stirred at 25° C. for 10 minutes. Then 4-(4-isocyanato-2,3-dihydro-1H-inden-5-yl)-2-methoxypyridine (Intermediate A39) (146 mg, 390.07 μmol, 1 eq) was added. The resulting mixture was stirred at 70° C. for 10 minutes. The mixture was concentrated in vacuo. The residue was purified by prep-HPLC (column: Waters Xbridge C18, 150 mm*50 mm*10 μm; mobile phase: [A: water (10 mM NH₄HCO₃); B: MeCN]; B %: 18%-48%, 11.5 min) to give the title compound (35.98 mg, 17% yield, 98% purity on LCMS) as a white solid.

¹H NMR (DMSO-d₆): δ 10.31 (br s, 1H), 8.16 (d, 1H), 8.00 (s, 1H), 7.32-7.26 (m, 4H), 7.24 (d, 2H), 7.14 (d, 1H), 6.93 (d, 1H), 6.75 (s, 1H), 3.86 (s, 3H), 3.56 (s, 2H), 3.26-3.22 (m, 2H), 2.93 (t, 2H), 2.79 (t, 2H), 2.47-2.40 (m, 4H), 2.02-1.97 (m, 2H), 1.81-1.76 (m, 2H) and 0.96 (t, 3H).

LCMS: m/z 523.3 (M+H)⁺ (ES⁺).

### Example 153: 3-(Benzyl(ethyl)amino)-N-((7-fluoro-5-(pyridin-4-yl)-2,3-dihydro-1H-inden-4-yl)carbamoyl)propane-1-sulfonamide

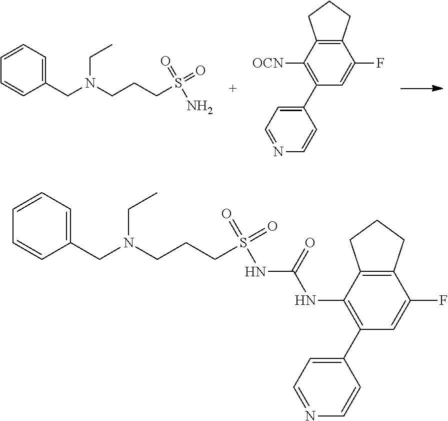

To a solution of 3-(benzyl(ethyl)amino)propane-1-sulfonamide (Intermediate P33) (101 mg, 393.30 μmol, 1 eq) in THF (1 mL) was added t-BuONa (38 mg, 393.30 μmol, 1 eq). The reaction mixture was stirred at 15° C. for 10 minutes. Then 4-(7-fluoro-4-isocyanato-2,3-dihydro-1H-inden-5-yl)pyridine (Intermediate A40) (100 mg, 393.30 μmol, 1 eq) was added. The resulting mixture was stirred at 15° C. for 10 minutes. The reaction mixture was concentrated in vacuo. The residue was purified by prep-HPLC (column: Phenomenex Gemini C18, 150 mm*25 mm*10 μm, mobile phase: [A: water (0.05% ammonium hydroxide v/v); B: MeCN]; B %: 5%-35%, 10 min) to give the title compound (67.23 mg, 33% yield, 100% purity on LCMS) as a pink solid.

¹H NMR (DMSO-d₆): δ 8.57 (d, 2H), 7.86 (br s, 1H), 7.38 (d, 2H), 7.31 (d, 4H), 7.26-7.23 (m, 1H), 7.03 (d, 1H), 3.56 (s, 2H), 3.18-3.15 (m, 2H), 2.96 (t, 2H), 2.85 (t, 2H), 2.47-2.42 (m, 4H), 2.10-2.03 (m, 2H), 1.76-1.70 (m, 2H) and 0.96 (t, 3H).

LCMS: m/z 511.3 (M+H)⁺ (ES⁺).

### Example 154: N-((2-(2-Cyanopyridin-4-yl)-4-fluoro-6-isopropylphenyl) carbamoyl)-3-methoxypropane-1-sulfonamide

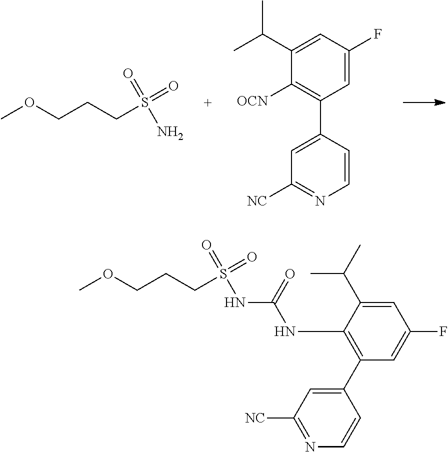

To a solution of 3-methoxypropane-1-sulfonamide (Intermediate P34) (65 mg, 426.62 μmol, 1.2 eq) in THF (1 mL) was added t-BuONa (34 mg, 355.51 μmol, 1 eq) and the mixture was stirred at 25° C. for m minutes. Then 4-(5-fluoro-2-isocyanato-3-isopropylphenyl)picolinonitrile (Intermediate A37) (100 mg, 355.51 μmol, 1 eq) was added. The resulting mixture was stirred at 70° C. for m minutes. The mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 11.5 min) to give the title compound (113.93 mg, 74% yield, m0% purity on LCMS) as a white solid.

¹HNMR (DMSO-d₆): δ 8.71 (d, 1H), 8.06 (s, 1H), 7.77 (s, 1H), 7.58 (s, 1H), 7.23-7.18 (m, 1H), 7.10 (d, 1H), 3.29-3.24 (m, 3H), 3.21 (s, 3H), 2.76-2.73 (m, 2H), 1.60-1.57 (m, 2H) and 1.16 (d, 6H).

LCMS: m/z 435.2 (M+H)⁺ (ES⁺).

### Example 155: N-((4-Fluoro-2-isopropyl-6-(2-methoxypyridin-4-yl)phenyl) carbamoyl)-3-methoxypropane-1-sulfonamide

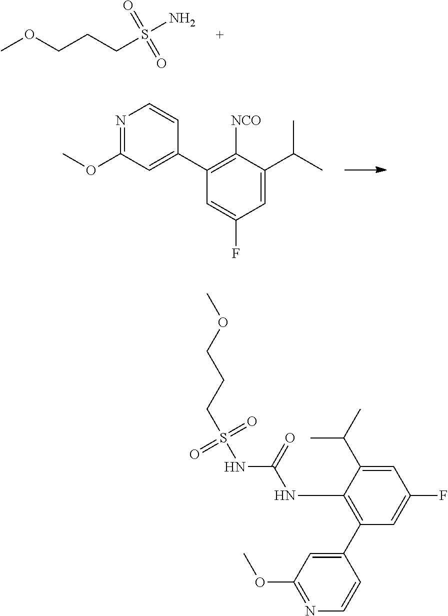

To a solution of 3-methoxypropane-1-sulfonamide (Intermediate P34) (64 mg, 419.14 μmol, 1.2 eq) in THF (1 mL) was added t-BuONa (34 mg, 349.28 μmol, 1 eq) and the mixture was stirred at 25° C. for m minutes. Then 4-(5-fluoro-2-isocyanato-3-isopropylphenyl)-2-methoxypyridine (Intermediate A38) (100 mg, 349.28 μmol, 1 eq) was added. The resulting mixture was stirred at 70° C. for m minutes. The mixture was concentrated in vacua. The residue was purified by prep-HPLC (column: Waters Xbridge C18, 150 mm*25 mm*5 μm; mobile phase: [A: water (0.05% ammonium hydroxide v/v); B: MeCN]; B %: 8%-38%, 11.5 min) to give the title compound (130.95 mg, 85% yield, 99.7% purity on LCMS) as a white solid.

¹H NMR (DMSO-d₆): δ 8.11 (d, 1H), 7.51 (s, 1H), 7.16 (d, 1H), 7.02-6.95 (m, 2H), 6.84 (s, 1H), 3.86 (s, 3H), 3.34-3.27 (m, 3H), 3.21 (s, 3H), 2.90-2.86 (m, 2H), 1.72-1.61 (m, 2H) and 1.15 (d, 6H).

LCMS: m/z 440.2 (M+H)⁺ (ES⁺).

### Example 156: 3-Methoxy-N-((5-(2-methoxypyridin-4-yl)-2,3-dihydro-1H-inden-4-yl)carbamoyl)propane-1-sulfonamide

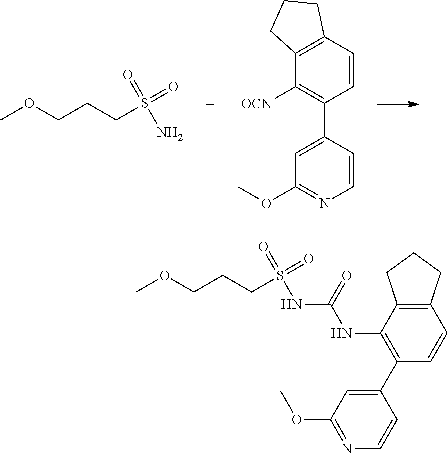

To a solution of 3-methoxypropane-1-sulfonamide (Intermediate P34) (72 mg, 469.98 μmol, 1.2 eq) in THF (1 mL) was added t-BuONa (38 mg, 391.65 μmol, 1 eq) and the mixture was stirred at 25° C. for 10 minutes. Then 4-(4-isocyanato-2,3-dihydro-1H-inden-5-yl)-2-methoxypyridine (Intermediate A39) (163 mg, 391.65 μmol, 1 eq) was added. The resulting mixture was stirred at 70° C. for 10 minutes. The mixture was concentrated in vacuo. The residue was purified by prep-HPLC (column: Waters Xbridge C18, 150 mm*25 mm*5 μm; mobile phase: [A: water (0.05% ammonium hydroxide v/v); B: MeCN]; B %: 5%-35%, 11.5 min) to give the title compound (15.13 mg, 9% yield, 100% purity on LCMS) as a white solid.

¹H NMR (DMSO-d₆): δ 10.34 (br s, 1H), 8.17 (d, 1H), 7.97 (br s, 1H), 7.24 (d, 1H), 7.14 (d, 1H), 6.94 (d, 1H), 6.76 (s, 1H), 3.88 (s, 3H), 3.37 (t, 2H), 3.26-3.20 (m, 5H), 2.95 (t, 2H), 2.81 (t, 2H), 2.06-2.02 (m, 2H) and 1.84-1.78 (m, 2H).

LCMS: m/z 420.2 (M+H)⁺ (ES⁺).

### Example 157: N-((7-Fluoro-5-(pyridin-4-yl)-2,3-dihydro-1H-inden-4-yl) carbamoyl)-3-methoxypropane-1-sulfonamide

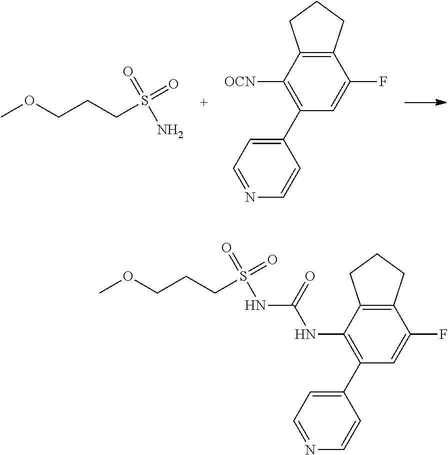

To a solution of 3-methoxypropane-1-sulfonamide (Intermediate P34) (60 mg, 393.30 μmol, 1 eq) in THF (1 mL) was added t-BuONa (38 mg, 393.30 μmol, 1 eq). The reaction mixture was stirred at 15° C. for 10 minutes. Then 4-(7-fluoro-4-isocyanato-2,3-dihydro-1H-inden-5-yl)pyridine (Intermediate A40) (100 mg, 393.30 μmol, 1 eq) was added. The resulting mixture was stirred at 15° C. for 20 minutes. The reaction mixture was concentrated in vacuo. The residue was purified by prep-HPLC (column: Phenomenex Gemini C18, 150 mm*25 mm*10 μm; mobile phase: [A: water (0.05% ammonium hydroxide v/v); B: MeCN]; B %: 0%-30%, 10 min) to give the title compound (62.33 mg, 38% yield, 100% purity on LCMS) as a pink solid.

¹H NMR (DMSO-d₆): δ 8.57 (d, 2H), 7.63 (br s, 1H), 7.40 (d, 2H), 7.00 (d, 1H), 3.37-3.34 (m, 2H), 3.23 (s, 3H), 3.07-3.04 (m, 2H), 2.97 (t, 2H), 2.87 (t, 2H), 2.11-2.05 (m, 2H) and 1.79-1.72 (m, 2H).

LCMS: m/z 408.2 (M+H)⁺ (ES⁺).

### Example 158: N-((2-(2-Cyanopyridin-4-yl)-4-fluoro-6-isopropylphenyl) carbamoyl)-1-(pyridin-3-yl)methanesulfonamide

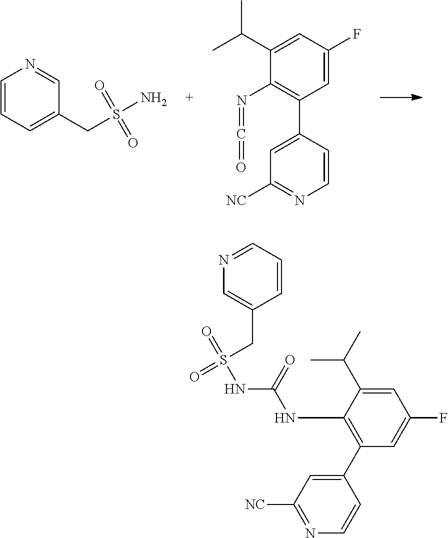

To a solution of pyridin-3-ylmethanesulfonamide (70 mg, 406.49 μmol, 1 eq) in THF (5 mL) was added t-BuONa (39 mg, 406.49 μmol, 1 eq) and 4-(5-fluoro-2-isocyanato-3-isopropylphenyl)picolinonitrile (Intermediate A37) (114 mg, 406.49 μmol, 1 eq). The mixture was stirred at 25° C. for 30 minutes. The reaction mixture was concentrated under reduced pressure. The residue was purified by prep-HPLC (column: Phenomenex Gemini C18, 150 mm*25 mm*10 μm; mobile phase: [A: water (0.05% ammonium hydroxide v/v); B: MeCN]; B %: 5%-35%, 11.5 min) to give the title compound (68 mg, 37% yield, 100% purity on LCMS) as a white solid.

¹H NMR (DMSO-d₆): δ 8.77 (d, 1H), 8.50 (d, 1H), 8.37 (s, 1H), 8.10 (s, 1H), 7.86 (br s, 1H), 7.79 (d, 1H), 7.61-7.45 (m, 1H), 7.33-7.27 (m, 2H), 7.19-7.02 (m, 1H), 4.31 (s, 2H), 3.24-3.18 (m, 1H) and 1.20-1.06 (m, 6H).

LCMS: m/z 454.3 (M+H)⁺ (ES⁺).

### Example 159: N-((4-Fluoro-2-isopropyl-6-(2-methoxypyridin-4-yl)phenyl) carbamoyl)-1-(pyridin-3-yl)methanesulfonamide

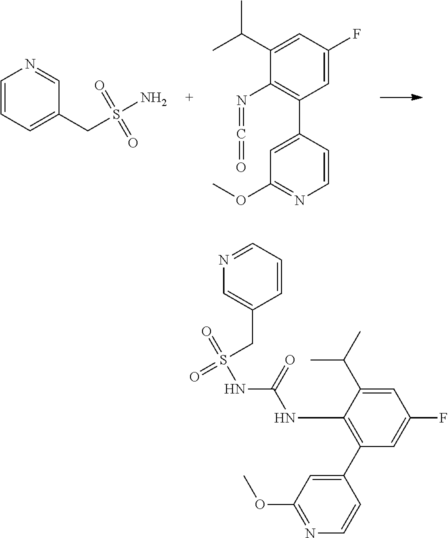

To a solution of pyridin-3-ylmethanesulfonamide (60 mg, 348.42 μmol, 1 eq) in THF (5 mL) was added t-BuONa (33 mg, 348.42 μmol, 1 eq) and 4-(5-fluoro-2-isocyanato-3-isopropylphenyl)-2-methoxypyridine (Intermediate A38) (100 mg, 348.42 μmol, 1 eq). The mixture was stirred at 25° C. for 30 minutes. The reaction mixture was concentrated under reduced pressure. The residue was purified by prep-HPLC (column: Phenomenex Gemini C18, 150 mm*25 mm*10 μm; mobile phase: [A: water (0.05% ammonium hydroxide v/v); B: MeCN]; B %: 8%-38%, 11.5 min) to give the title compound (70 mg, 44% yield, 100% purity on LCMS) as a white solid.

¹H NMR (DMSO-d₆): δ 8.50 (d, 1H), 8.41 (s, 1H), 8.16 (d, 1H), 7.61 (br s, 1H), 7.50 (d, 1H), 7.33-7.30 (m, 1H), 7.21 (d, 1H), 7.06-7.00 (m, 2H), 6.87 (s, 1H), 4.33 (s, 2H), 3.85 (s, 3H), 3.22-3.17 (t, 1H) and 1.20-1.04 (m, 6H).

LCMS: m/z 459.3 (M+H)⁺ (ES⁺).

### Example 160: N-((5-(2-Methoxypyridin-4-yl)-2,3-dihydro-1H-inden-4-yl) carbamoyl)-1-(pyridin-3-yl)methanesulfonamide

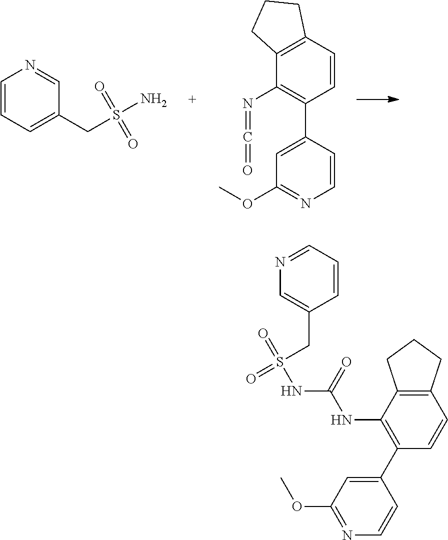

To a solution of pyridin-3-ylmethanesulfonamide (70 mg, 406.49 μmol, 1 eq) in THF (5 mL) was added t-BuONa (39 mg, 406.49 μmol, 1 eq) and 4-(4-isocyanato-2,3-dihydro-1H-inden-5-yl)-2-methoxypyridine (Intermediate A39) (108 mg, 406.49 μmol, 1 eq). The mixture was stirred at 25° C. for 30 minutes. The reaction mixture was concentrated under reduced pressure. The residue was purified by prep-HPLC (column: Phenomenex Gemini C18, 150 mm*25 mm*10 μm; mobile phase: [A: water (0.05% ammonium hydroxide v/v); B: MeCN]; B %: 5%-35%, 11.5 min) to give the title compound (65 mg, 36% yield, 100% purity on LCMS) as a white solid.

¹H NMR (DMSO-d₆): δ 8.52 (d, 1H), 8.46 (d, 1H), 8.16 (d, 1H), 7.61 (d, 1H), 7.37-7.34 (m, 1H), 7.17 (d, 1H), 7.10 (d, 1H), 6.97-6.95 (m, 1H), 6.78 (s, 1H), 4.45 (s, 2H), 3.86 (s, 3H), 2.93 (t, 2H), 2.83 (t, 2H) and 2.07-1.98 (m, 2H).

LCMS: m/z 439.3 (M+H)⁺ (ES⁺).

### Example 161: N-((7-Fluoro-5-(pyridin-4-yl)-2,3-dihydro-1H-inden-4-yl) carbamoyl)-1-(pyridin-3-yl)methanesulfonamide

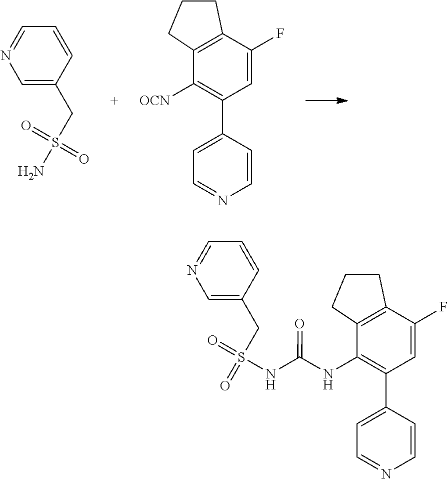

To a solution of pyridin-3-ylmethanesulfonamide (68 mg, 393.30 μmol, 1 eq) in THF (2 mL) was added t-BuONa (38 mg, 393.30 μmol, 1 eq). Then the reaction mixture was stirred at 25° C. for 10 minutes. A solution of 4-(7-fluoro-4-isocyanato-2,3-dihydro-1H-inden-5-yl)pyridine (Intermediate A40) (100 mg, 393.30 μmol, 1 eq) in THF (2.5 mL) was added. The resulting mixture was stirred at 25° C. for 30 minutes. The reaction mixture was concentrated under reduced pressure. The residue was purified by prep-HPLC (column: Xtimate C18, 250 mm*50 mm*10 μm; mobile phase: [A: water (0.05% ammonium hydroxide v/v); B: MeCN]; B %: 1%-31%, 10 min) to give the title compound (22.34 mg, 13%) as a white solid.

¹H NMR (DMSO-d₆): δ 8.57 (d, 2H), 8.49-8.45 (m, 2H), 7.59 (d, 1H), 7.39 (d, 2H), 7.34-7.30 (m, 1H), 6.96 (d, 1H), 4.34 (s, 2H), 2.95 (t, 2H), 2.87 (t, 2H) and 210-2.05 (m, 2H).

LCMS: m/z 427.2 (M+H)⁺ (ES⁺).

### Example 162: N-((2-(2-Cyanopyridin-4-yl)-4-fluoro-6-isopropylphenyl) carbamoyl)-1-(1-methylpyrrolidin-3-yl) methanesulfonamide

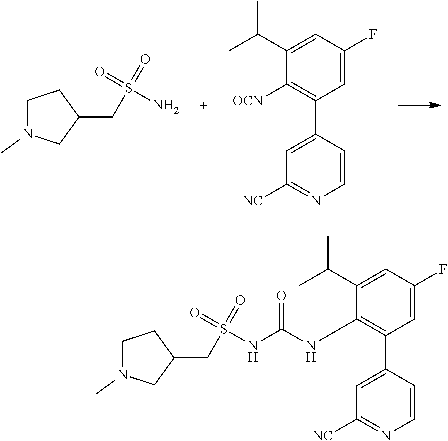

A solution of (1-methylpyrrolidin-3-yl)methanesulfonamide (Intermediate P31) (180 mg, crude) and t-BuONa (97 mg, 1.01 mmol, 1 eq) in THF (3 mL) was stirred at 25° C. for 10 minutes. Then 4-(5-fluoro-2-isocyanato-3-isopropylphenyl)picolinonitrile (Intermediate A37) (57 mg, 201.96 μmol, 0.2 eq) was added. The resulting mixture was stirred at 25° C. for 30 minutes. The reaction mixture was concentrated in vacuo. The residue was purified by prep-HPLC (column: Phenomenex Gemini C18, 150 mm*25 mm*5 μm, mobile phase: [A: water (0.05% ammonium hydroxide v/v); B: MeCN], B %: 10%-40%, 10.0 min) to give the title compound (17.51 mg, 4% yield, 100% purity on LCMS) as a white solid.

¹H NMR (DMSO-d₆+D₂O): δ 8.70 (d, 1H), 8.00 (s, 1H), 7.74 (s, 1H), 7.17 (dd, 1H), 7.06 (dd, 1H), 3.26-3.15 (m, 2H), 3.10-3.01 (m, 2H), 2.95-2.80 (m, 2H), 2.77-2.72 (m, 1H), 2.67 (s, 3H), 2.45-2.40 (m, 1H), 2.10-1.98 (m, 1H), 1.62-1.51 (m, 1H) and 1.13 (d, 6H).

LCMS: m/z 460.2 (M+H)⁺ (ES⁺).

### Example 163: N-((4-Fluoro-2-isopropyl-6-(2-methoxypyridin-4-yl)phenyl) carbamoyl)-1-(1-methylpyrrolidin-3-yl) methanesulfonamide

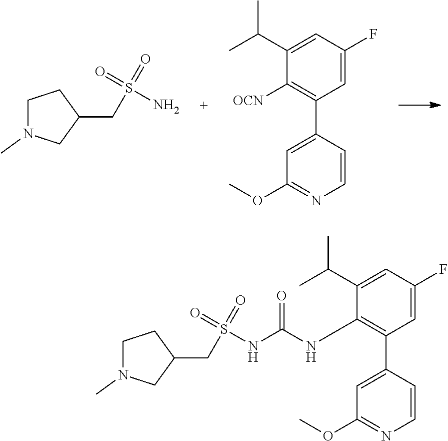

A solution of (1-methylpyrrolidin-3-yl)methanesulfonamide (Intermediate P31) (180 mg, crude) and t-BuONa (97 mg, 1.01 mmol, 1 eq) in THF (3 mL) was stirred at 25° C. for 10 minutes. Then 4-(5-fluoro-2-isocyanato-3-isopropylphenyl)-2-methoxypyridine (Intermediate A38) (58 mg, 201.96 μmol, 0.2 eq) was added. The resulting mixture was stirred at 25° C. for 30 minutes and then concentrated in vacua. The residue was purified by prep-HPLC (column: Phenomenex Gemini C18, 150 mm*25 mm*5 μm, mobile phase: [A: water (0.05% ammonium hydroxide v/v); B: MeCN], B %: 12%-42%, 10.0 min) to give the title compound (4.92 mg, 1% yield, 100% purity on LCSM) as a white solid.

¹H NMR (DMSO-d6+D20): δ 8.12 (d, 1H), 7.14-7.11 (m, 1H), 7.04-7.02 (m, 1H), 6.96-6.93 (m, 1H), 6.85-6.83 (m, 1H), 3.86 (s, 3H), 3.30-3.14 (m, 2H), 3.05-2.98 (m, 3H), 2.92-2.83 (m, 2H), 2.63 (s, 3H), 2.60-2.57 (m, 1H), 2.04-2.00 (m, 1H), 1.61-1.57 (m, 1H) and 1.14 (d, 6H).

LCMS: m/z 465.2 (M+H)⁺ (ES⁺).

### Example 164: N-((5-(2-Methoxypyridin-4-yl)-2,3-dihydro-1H-inden-4-yl) carbamoyl)-1-(1-methylpyrrolidin-3-yl) methanesulfonamide

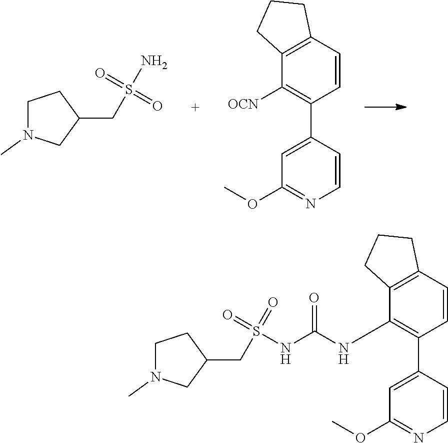

A solution of (1-methylpyrrolidin-3-yl)methanesulfonamide (Intermediate P31) (180 mg, crude) and t-BuONa (97 mg, 1.01 mmol, 1 eq) in THF (3 mL) was stirred at 25° C. for 10 minutes. Then 4-(4-isocyanato-2,3-dihydro-1H-inden-5-yl)-2-methoxypyridine (Intermediate A39) (54 mg, 201.96 μmol, 0.2 eq) was added. The resulting mixture was stirred at 25° C. for 30 minutes and then concentrated in vacua. The residue was purified by prep-HPLC (column: Phenomenex Gemini C18, 150 mm*25 mm*5 μm, mobile phase: [A: water (0.05% ammonium hydroxide v/v); B: MeCN], B %: 10%-40%, 10.0 min) to give the title compound (5.47 mg, 1% yield, 100% purity on LCMS) as a white solid.

¹H NMR (DMSO-d₆+D₂O): δ 8.09 (d, 1H), 7.11 (d, 1H), 7.04 (d, 1H), 6.98 (d, 1H), 6.78 (s, 1H), 3.84 (s, 3H), 3.28-3.21 (m, 1H), 3.15-3.01 (m, 3H), 2.95-2.90 (m, 1H), 2.89-2.86 (m, 3H), 2.84-2.78 (m, 2H), 2.64 (s, 3H), 2.61-2.55 (m, 1H), 2.11-1.96 (m, 3H) and 1.66-1.55 (m, 1H).

LCMS: m/z 445.2 (M+H)⁺ (ES⁺).

### Example 165: N-((7-Fluoro-5-(pyridin-4-yl)-2,3-dihydro-1H-inden-4-yl) carbamoyl)-1-(1-methylpyrrolidin-3-yl) methanesulfonamide

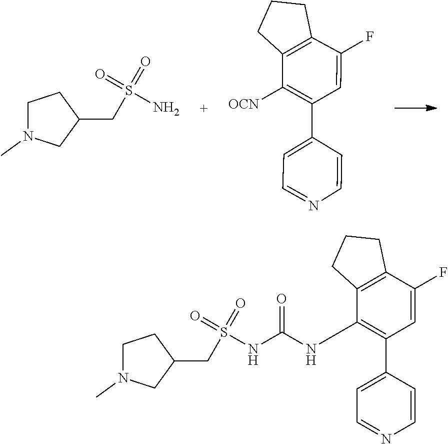

To a solution of (1-methylpyrrolidin-3-yl)methanesulfonamide (Intermediate P31) (180 mg, 1.01 mmol, 5 eq) in THF (2 mL) was added t-BuONa (97 mg, 1.01 mmol, 5 eq) and the mixture was stirred at 25° C. for 10 minutes. Then 4-(7-fluoro-4-isocyanato-2,3-dihydro-1H-inden-5-yl)pyridine (Intermediate A40) (51 mg, 201.96 μmol, 1 eq) in THF (1.5 mL) was added. The reaction mixture was stirred at 25° C. for 30 minutes. Most of the solvent was evaporated under reduced pressure. The residue was purified by prep-HPLC (Phenomenex Gemini C18, 150 mm*25 mm*5 μm, mobile phase: [A: water (0.05% ammonium hydroxide v/v); B: MeCN), B %: 8%-38%, 10 min) to give the title compound (5.52 mg, 6% yield, 100% purity on LCMS) as a white solid.

¹H NMR (DMSO-d₆): δ 8.55 (d, 2H), 7.41 (d, 2H), 7.40 (br s, 1H), 6.95 (d, 1H), 3.12-3.08 (m, 2H), 2.97-2.85 (m, 7H), 2.75-2.71 (m, 1H), 2.58 (s, 3H), 2.53-2.50 (m, 1H), 2.09-2.00 (m, 3H) and 1.59-1.57 (m, 1H).

LCMS: m/z 433.2 (M+H)⁺ (ES⁺).

### Example 166: 3-(N-((4-Fluoro-2-(2-isopropoxypyridin-4-yl)-6-isopropyl-phenyl)carbamoyl)sulfamoyl)-N,N-bis(2-methoxyethyl)-1-methyl-1H-pyrazole-5-carboxamide, sodium salt

### Step A: 3-(N-((4-Fluoro-2-(2-isopropoxypyridin-4-yl)-6-isopropylphenyl)carbamoyl) sulfamoyl)-N,N-bis(2-methoxyethyl)-1-methyl-1H-pyrazole-5-carboxamide

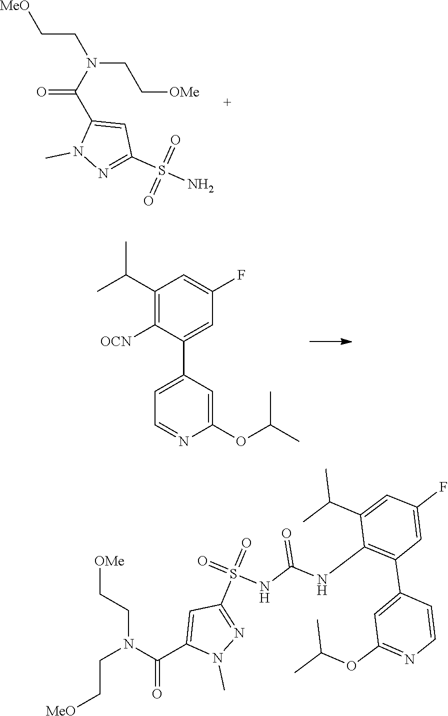

A solution of N,N-bis(2-methoxyethyl)-1-methyl-3-sulfamoyl-1H-pyrazole-5-carboxamide (Intermediate P35) (2.2 g, 6.87 mmol, 1 eq), 4-(5-fluoro-2-isocyanato-3-isopropylphenyl)-2-isopropoxypyridine (Intermediate A46) (2.16 g, 6.87 mmol, 1 eq) and t-BuONa (659 mg, 6.87 mmol, 1 eq) in THF (100 mL) was stirred at 25° C. for 30 minutes. The reaction mixture was concentrated in vacuo. The residue was purified by reversed phase flash chromatography (column: Welch Ultimate XB_C18, 41 mm*235 mm*20/40 μm, mobile phase: [A: water (10 mM NH₄HCO₃); B: MeCN]; B %: 0%-30%, 35 min) to give the title compound (2.5 g, 56% yield, 98% purity on LCMS) as a white solid.

¹H NMR (DMSO-d₆): δ 11.10 (br s, 1H), 8.06 (d, 1H), 7.79 (br s, 1H), 7.18 (d, 1H), 7.02 (d, 1H), 6.83-6.72 (m, 2H), 6.70 (s, 1H), 5.29-5.23 (m, 1H), 3.83 (s, 3H), 3.64-3.61 (m, 2H), 3.55-3.50 (m, 4H), 3.45-3.40 (m, 2H), 3.28 (s, 3H), 3.14 (s, 3H), 3.03-3.00 (m, 1H), 1.30 (d, 6H) and 1.09-1.05 (m, 6H).

LCMS: m/z 635.4 (M+H)⁺ (ES⁺).

### Step B: 3-(N-((4-Fluoro-2-(2-isopropoxypyridin-4-yl)-6-isopropylphenyl)carbamoyl) sulfamoyl)-N,N-bis(2-methoxyethyl)-1-methyl-1H-pyrazole-5-carboxamide, sodium salt

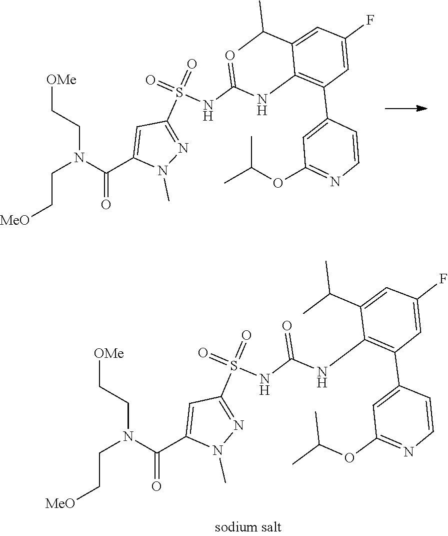 [SMILES: COCCN(CCOC)C(=O)c1cc(S(=O)(=O)NC(=O)Nc2c(-c3ccnc(OC(C)C)c3)cc(F)cc2C(C)C)nn1C ; COCCN(CCOC)C(=O)c1cc(S(=O)(=O)NC(=O)Nc2c(-c3ccnc(OC(C)C)c3)cc(F)cc2C(C)C)nn1C]

To a solution of 3-(N-((4-fluoro-2-(2-isopropoxypyridin-4-yl)-6-isopropylphenyl) carbamoyl)sulfamoyl)-N,N-bis(2-methoxyethyl)-1-methyl-1H-pyrazole-5-carboxamide (2.5 g, 3.94 mmol, 1 eq, free form) in THF (100 mL) was added with t-BuONa (378 mg, 3.94 mmol, 1 eq). The reaction mixture was stirred at 25° C. for 1 hour and then concentrated in vacuo. The residue was triturated with isopropyl ether (20 mL) to give the title compound (2.2 g, 85% yield, 99% purity on LCMS, sodium salt) as a white solid.

¹H NMR (DMSO-d₆): δ 7.99-7.88 (m, 1H), 7.53-7.40 (m, 1H), 7.15-7.08 (m, 1H), 6.94-6.82 (m, 2H), 6.68 (s, 1H), 6.51-6.44 (m, 1H), 5.28-5.22 (m, 1H), 3.75 (s, 3H), 3.74-3.56 (m, 6H), 3.45-3.38 (m, 2H), 3.29 (s, 3H), 3.17 (s, 3H), 3.12-3.07 (m, 1H), 1.29 (d, 6H) and 1.20-1.04 (m, 6H).

LCMS: m/z 635.1 (M+H)⁺ (ES⁺).

### Example 167: N,N-Bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide, sodium salt

### Step A: N,N-Bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide

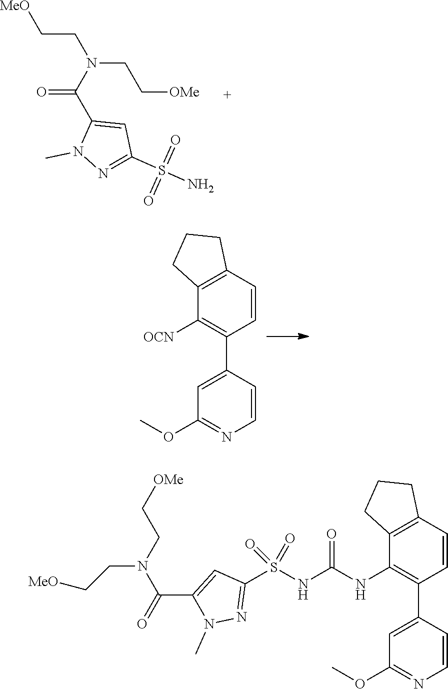

A solution of N,N-bis(2-methoxyethyl)-1-methyl-3-sulfamoyl-1H-pyrazole-5-carboxamide (Intermediate P35) (2.56 g, 7.99 mmol, 1 eq) and t-BuONa (768 mg, 7.99 mmol, 1 eq) in THF (200 mL) was stirred at 25° C. for 30 minutes. Then 4-(4-isocyanato-2,3-dihydro-1H-inden-5-yl)-2-methoxypyridine (Intermediate A39) (3.34 g, 8.79 mmol, 1.1 eq) was added. The reaction mixture was stirred at 70° C. for 2 hours and then concentrated in vacuo. The residue was purified by reversed phase flash chromatography (column: Welch Ultimate XB_C18, 41 mm*235 mm*20/40 μm, mobile phase: [A: water (0.05% ammonium hydroxide); B: MeCN]; B %: 0%-30%, 35 min) to give the title compound (1.35 g, 29% yield, 99% purity on LCMS) as a white solid.

¹H NMR (DMSO-d₆): δ 8.08 (d, 1H), 7.14-7.11 (m, 1H), 7.07-7.05 (m, 1H), 6.91 (d, 1H), 6.74 (s, 1H), 6.60 (s, 1H), 3.86 (s, 3H), 3.78 (s, 3H), 3.64-3.62 (m, 2H), 3.56-3.54 (m, 4H), 3.39-3.37 (m, 2H), 3.28 (s, 3H), 3.14 (s, 3H), 2.89 (t, 2H), 2.71 (t, 2H) and 1.99-1.94 (m, 2H).

LCMS: m/z 587.3 (M+H)⁺ (ES⁺).

### Step B: N,N-Bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide, sodium salt

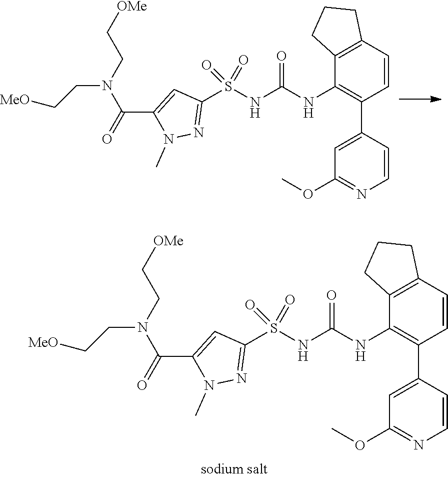 [SMILES: COCCN(CCOC)C(=O)c1cc(S(=O)(=O)NC(=O)Nc2c(-c3ccnc(OC)c3)ccc3c2CCC3)nn1C ; COCCN(CCOC)C(=O)c1cc(S(=O)(=O)NC(=O)Nc2c(-c3ccnc(OC)c3)ccc3c2CCC3)nn1C]

To a solution of N,N-bis(2-methoxyethyl)-3-(N-((5-(2-methoxypyridin-4-yl)-2,3-dihydro-1H-inden-4-yl)carbamoyl)sulfamoyl)-1-methyl-1H-pyrazole-5-carboxamide (1.35 g, 2.30 mmol, 1 eq, free form) in THF (20 mL) was added with t-BuONa (221 mg, 2.30 mmol, 1 eq). The reaction mixture was stirred at 25° C. for 1 hour and then concentrated in vacuo. The residue was triturated with isopropyl ether (20 mL) to give the title compound (1.2 g, 85% yield, 99% purity on HPLC) as a white solid.

¹H NMR (DMSO-d₆): δ 8.05 (d, 1H), 7.30 (br s, 1H), 7.04 (dd, 2H), 6.92 (d, 1H), 6.76 (s, 1H), 6.48 (d, 1H), 3.85 (s, 3H), 3.75 (s, 3H), 3.64-3.62 (m, 2H), 3.56-3.53 (m, 4H), 3.39-3.37 (m, 2H), 3.29 (s, 3H), 3.15 (s, 3H), 2.87 (t, 2H), 2.73-2.70 (m, 2H) and 1.98-1.91 (m, 2H).

LCMS: m/z 587.1 (M+H)⁺ (ES⁺).

### Example 168: 3-(N-((4-Fluoro-2-(2-isopropoxypyridin-4-yl)-6-isopropyl-phenyl)carbamoyl)sulfamoyl)-N,N,1-trimethyl-1H-pyrazole-5-carboxamide, sodium salt

### Step A: 3-(N-((4-Fluoro-2-(2-isopropoxypyridin-4-yl)-6-isopropylphenyl)carbamoyl) sulfamoyl)-N,N,1-trimethyl-1H-pyrazole-5-carboxamide

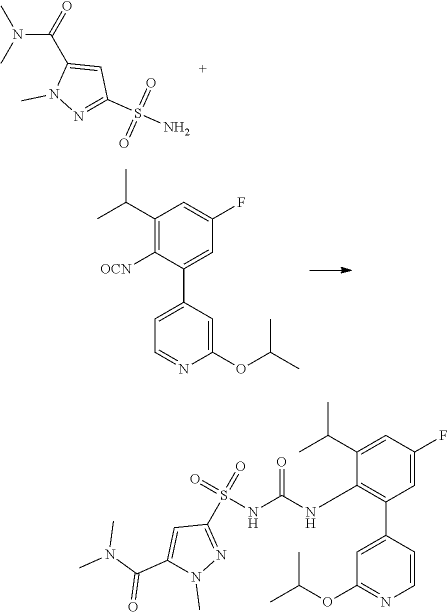

To a solution of N,N,1-trimethyl-3-sulfamoyl-1H-pyrazole-5-carboxamide (Intermediate P36) (1.7 g, 7.32 mmol, 1 eq) in THF (20 mL) was added t-BuONa (703 mg, 7.32 mmol, 1 eq) at 25° C. and stirred for 0.5 hour. Then 4-(5-fluoro-2-isocyanato-3-isopropylphenyl)-2-isopropoxypyridine (Intermediate A46) (2.30 g, 7.32 mmol, 1 eq) was added and the resulting mixture was stirred for 0.5 hour. The mixture was concentrated in vacuo. The residue was purified by prep-HPLC (column: Welch Ultimate XB_C18, 41 mm*235 mm*20/40 μm; mobile phase: [water (10 mM NH₄HCO₃)-ACN]; B %: 0%-30%, 35 min) to give the title compound (2.34 g, 59% yield, 98% purity on HPLC) as a white solid.

¹H NMR (DMSO-d₆): δ 8.03 (d, 1H), 7.65 (br s, 1H), 7.16 (d, 1H), 6.98 (d, 1H), 6.85 (d, 1H), 6.74 (s, 1H), 6.70 (s, 1H), 5.30-5.21 (m, 1H), 3.89 (s, 3H), 3.09-3.03 (m, 1H), 3.00 (s, 6H), 1.30 (d, 6H) and 1.07 (d, 6H).

LCMS: m/z 547.4 (M+H)⁺ (ES⁺).

### Step B: 3-(N-((4-Fluoro-2-(2-isopropoxypyridin-4-yl)-6-isopropylphenyl)carbamoyl) sulfamoyl)-N,N,1-trimethyl-1H-pyrazole-5-carboxamide, sodium salt

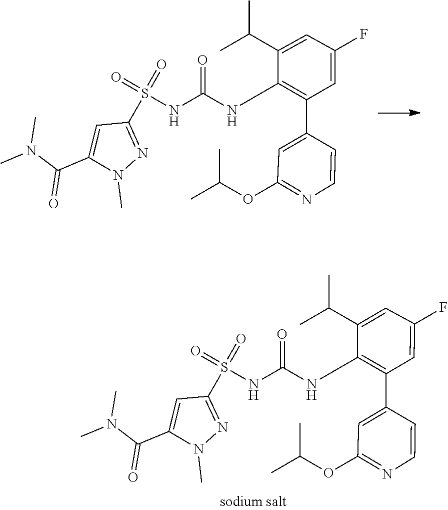 [SMILES: CC(C)Oc1cc(-c2cc(F)cc(C(C)C)c2NC(=O)NS(=O)(=O)c2cc(C(=O)N(C)C)n(C)n2)ccn1 ; CC(C)Oc1cc(-c2cc(F)cc(C(C)C)c2NC(=O)NS(=O)(=O)c2cc(C(=O)N(C)C)n(C)n2)ccn1]

To a solution of 3-(N-((4-fluoro-2-(2-isopropoxypyridin-4-yl)-6-isopropylphenyl) carbamoyl)sulfamoyl)-N,N,1-trimethyl-1H-pyrazole-5-carboxamide (1.71 g, 3.13 mmol, 1 eq, free form) in THF (40 mL) was added t-BuONa (300 mg, 3.13 mmol, 1 eq) at 25° C. Then the mixture was stirred for 1 hour. The mixture was concentrated in vacuo. The residue was triturated with MTBE (100 mL). The solid was dissolved in water (100 mL) and then lyophilized to give the title compound (1.60 g, 90% yield, 99.9% purity on HPLC) as a white solid.

¹H NMR (DMSO-d₆): δ 7.95 (d, 1H), 7.37 (br s, 1H), 7.09 (d, 1H), 6.93-6.90 (m, 2H), 6.69 (s, 1H), 6.53 (s, 1H), 5.29-5.22 (m, 1H), 3.83 (s, 3H), 3.15-3.09 (m, 1H), 3.01 (d, 6H), 1.29 (d, 6H) and 1.05 (d, 6H).

LCMS: m/z 547.3 (M+H)⁺ (ES⁺).

### Example 169: 3-(N-((5-(2-Methoxypyridin-4-yl)-2,3-dihydro-1H-inden-4-yl) carbamoyl)sulfamoyl)-N,N,1-trimethyl-1H-pyrazole-5-carboxamide, sodium salt

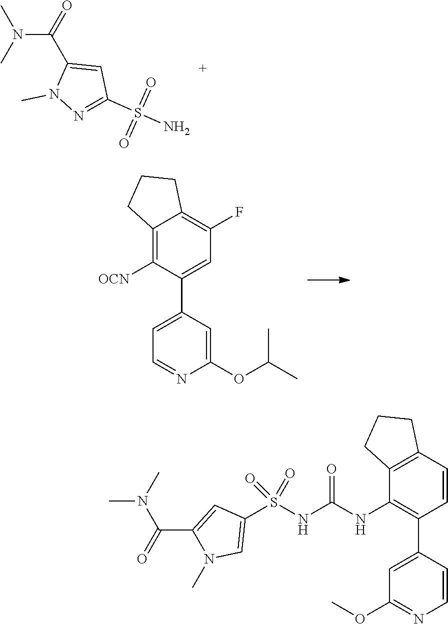

A solution of N,N,1-trimethyl-3-sulfamoyl-1H-pyrazole-5-carboxamide (Intermediate P36) (6.59 g, 28.39 mmol, 0.9 eq) and t-BuONa (3.33 g, 34.70 mmol, 1.1 eq) in THF (200 mL) was stirred at 16° C. for 0.5 hour. Then 4-(4-isocyanato-2,3-dihydro-1H-inden-5-yl)-2-methoxypyridine (Intermediate A39) (8.4 g, 31.54 mmol, 1 eq) was added. The reaction mixture was stirred at 16° C. for 0.5 hour and then filtered. The filter cake was washed with MeCN (125 mL). Then the solid was dissolved in H₂O (100 mL) and filtered. The filtrate was lyophilized to give the title compound (8.02 g, 49% yield, 99.54% purity on LCMS, Na salt) as a white solid.

¹H NMR (DMSO-d₆): δ 8.02 (d, 1H), 7.42 (br s, 1H), 7.10-7.02 (m, 2H), 6.89 (dd, 1H), 6.74 (s, 1H), 6.59 (s, 1H), 3.84 (d, 6H), 3.02 (d, 6H), 2.87 (t, 2H), 2.72 (t, 2H) and 1.97-1.90 (m, 2H).

LCMS: m/z 499.3 (M+H)⁺ (ES⁺).

### Example 170: 1-Cyclopropyl-N-((7-fluoro-5-(2-methoxypyridin-4-yl)-2,3-dihydro-1H-inden-4-yl)carbamoyl)-1H-pyrazole-3-sulfonamide, sodium salt

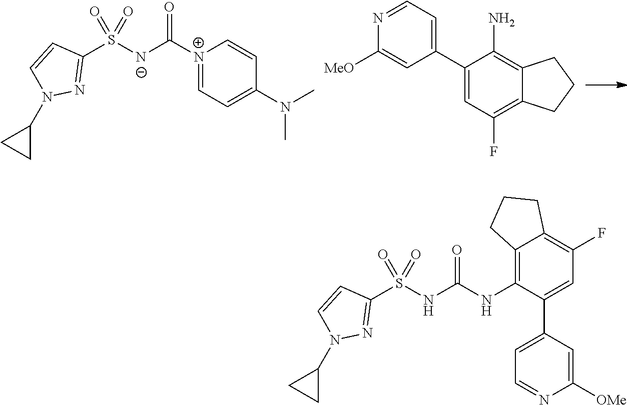

7-Fluoro-5-(2-methoxypyridin-4-yl)-2,3-dihydro-1H-inden-4-amine (Intermediate A34) (60 mg, 0.232 mmol) and ((1-cyclopropyl-1H-pyrazol-3-yl)sulfonyl)(4-(dimethylamino)pyridin-1-ium-1-carbonyl)amide (Intermediate P37) (80 mg, 0.239 mmol) were suspended in MeCN (2 mL) and the mixture was heated to 50° C. for 1 hour. The MeCN was removed in vacuo. The residue was dissolved in DMSO (2 mL) and purified by basic prep-HPLC. After concentration of product containing fractions, the free acid (55 mg, 50%) was isolated as a colourless solid. This solid was dissolved in 0.1 M aq NaOH (1.17 mL, 1 eq) and freeze dried overnight to afford the title compound (50 mg, 43%) as a colourless solid.

¹H NMR (DMSO-d6) δ 8.09-8.03 (m, 1H), 7.70 (d, J=9.9 Hz, 1H), 7.32 (s, 1H), 6.94 (s, 1H), 6.90 (d, J=9.3 Hz, 1H), 6.79 (s, 1H), 6.31-6.24 (m, 1H), 3.87 (s, 3H), 3.76-3.66 (m, 1H), 2.91 (t, J=7.5 Hz, 2H), 2.77 (t, J=7.5 Hz, 2H), 2.02 (p, J=7.5 Hz, 2H), 1.08-1.00 (m, 2H), 0.99-0.90 (m, 2H).

LCMS; m/z 472.2 (M+H)⁺ (ES⁺); 470.0 (M−H)⁻ (ES⁻).

### Example 171: 1-Cyclopropyl-N-((7-cyclopropyl-5-(2-methoxypyridin-4-yl)-2,3-dihydro-1H-inden-4-yl)carbamoyl)-1H-pyrazole-3-sulfonamide, sodium salt

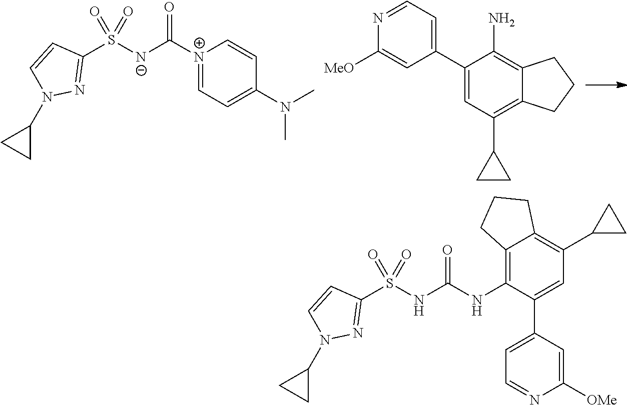

Prepared according to the general procedure of 1-cyclopropyl-N-((7-fluoro-5-(2-methoxypyridin-4-yl)-2,3-dihydro-1H-inden-4-yl)carbamoyl)-1H-pyrazole-3-sulfonamide, sodium salt (Example 170) from ((1-cyclopropyl-1H-pyrazol-3-yl)sulfonyl)(4-(dimethylamino)pyridin-1-ium-1-carbonyl)amide (Intermediate P37) and 7-cyclopropyl-5-(2-methoxypyridin-4-yl)-2,3-dihydro-1H-inden-4-amine (Intermediate A47) to afford the title compound (36 mg, 39%) as a white solid.

¹H NMR (DMSO-d6) δ 8.01 (d, J=5.3 Hz, 1H), 7.70 (d, J=2.3 Hz, 1H), 7.24 (s, 1H), 6.90 (dd, J=5.3, 1.5 Hz, 1H), 6.74 (d, J=1.3 Hz, 1H), 6.54 (s, 1H), 6.28 (d, J=2.3 Hz, 1H), 3.85 (s, 3H), 3.76-3.67 (m, 1H), 2.95 (t, J=7.5 Hz, 2H), 2.73 (t, J=7.5 Hz, 2H), 1.98 (p, J=7.6 Hz, 2H), 1.90-1.80 (m, 1H), 1.08-1.01 (m, 2H), 0.98-0.92 (m, 2H), 0.90-0.84 (m, 2H), 0.67-0.59 (m, 2H).

LCMS; m/z 494.1 (M+H)⁺ (ES⁺).

### Example 172: 1-Cyclobutyl-N-((7-fluoro-5-(2-methoxypyridin-4-yl)-2,3-dihydro-1H-inden-4-yl)carbamoyl)-1H-pyrazole-3-sulfonamide, sodium salt

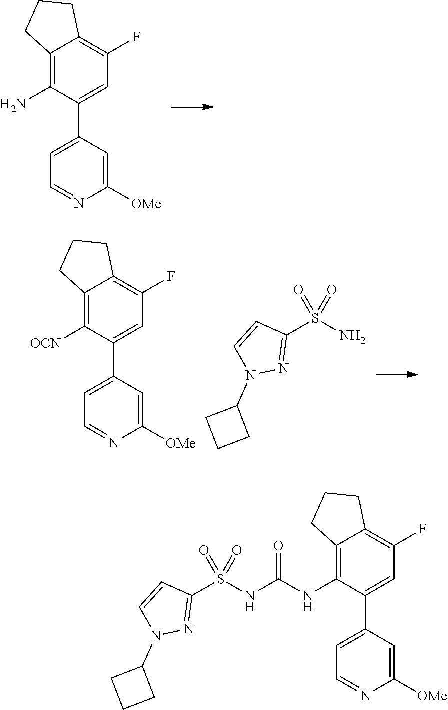

7-Fluoro-5-(2-methoxypyridin-4-yl)-2,3-dihydro-1H-inden-4-amine (Intermediate A34) (154 mg, 0.596 mmol) was dissolved in DCM (5 mL). Saturated aqueous NaHCO₃ (3 mL) was added, followed by a solution of triphosgene (70 mg, 0.236 mmol) in DCM (1 mL). The biphasic mixture was stirred at room temperature for 1 hour. Then the organic phase was dried by passing through a hydrophobic frit and concentrated in vacua to afford crude 4-(7-fluoro-4-isocyanato-2,3-dihydro-1H-inden-5-yl)-2-methoxypyridine (85 mg, 50%) as a yellow solid that was used without further purification.

1-Cyclobutyl-1H-pyrazole-3-sulfonamide (Intermediate P38) (60 mg, 0.298 mmol) was dissolved in dry THF (2 mL) and sodium tert-butoxide (2 M in THF) (160 μl, 0.320 mmol) was added. The mixture was stirred at room temperature for 1 hour, before a solution of 4-(7-fluoro-4-isocyanato-2,3-dihydro-1H-inden-5-yl)-2-methoxypyridine (85 mg, 0.298 mmol) in THF (1 mL) was added. The mixture was stirred at room temperature overnight. Then the solvent removed in vacuo, the residue dissolved in DMSO (2 mL) and purified by basic prep-HPLC. The free acid was isolated as a colourless solid which was dissolved in 0.1 M aq NaOH (0.8 mL, 0.08 mmol, 1 eq) and the solution freeze dried to afford the title compound (37 mg, 24%) as a colourless solid.

¹H NMR (DMSO-d6) δ 8.04 (d, J=5.1 Hz, 1H), 7.77-7.72 (m, 1H), 7.33 (s, 1H), 6.94 (d, J=4.6 Hz, 1H), 6.90 (d, J=9.3 Hz, 1H), 6.80 (s, 1H), 6.32-6.29 (m, 1H), 4.82 (p, J=8.3 Hz, 1H), 3.86 (s, 3H), 2.91 (t, J=7.4 Hz, 2H), 2.76 (t, J=7.7 Hz, 2H), 2.49-2.41 (m, 2H), 2.39-2.31 (m, 2H), 2.00 (p, J=7.6 Hz, 2H), 1.83-1.70 (m, 2H).

LCMS; m/z 486.1 (M+H)⁺ (ES⁺); 484.3 (M−H)⁻ (ES⁻).

### Example 173: 1-(1-(Azetidin-1-yl)-2-methylpropan-2-yl)-N-((7-fluoro-5-(2-methoxypyridin-4-yl)-2,3-dihydro-1H-inden-4-yl)carbamoyl)-1H-pyrazole-3-sulfonamide, sodium salt

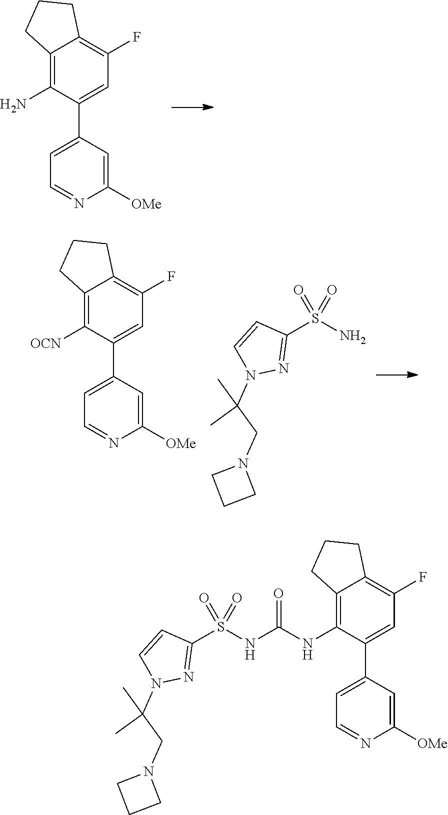

Prepared according to the general procedure of 1-cyclobutyl-N-((7-fluoro-5-(2-methoxypyridin-4-yl)-2,3-dihydro-1H-inden-4-yl)carbamoyl)-1H-pyrazole-3-sulfonamide, sodium salt (Example 172) from 1-(1-(azetidin-1-yl)-2-methylpropan-2-yl)-1H-pyrazole-3-sulfonamide (Intermediate P39) and 7-fluoro-5-(2-methoxypyridin-4-yl)-2,3-dihydro-1H-inden-4-amine (Intermediate A34) to afford the title compound (60 mg, 37%) as a colourless solid.

¹H NMR (DMSO-d6) δ 8.07 (d, J=5.5 Hz, 1H), 7.70-7.66 (m, 1H), 7.34 (s, 1H), 6.96 (d, J=4.6 Hz, 1H), 6.90 (d, J=9.3 Hz, 1H), 6.81 (s, 1H), 6.30 (q, J=2.1 Hz, 1H), 3.87 (s, 3H), 2.95 (t, J=7.0 Hz, 4H), 2.91 (t, J=7.5 Hz, 2H), 2.75 (t, J=7.4 Hz, 2H), 2.64 (s, 2H), 1.99 (p, J=7.6 Hz, 2H), 1.82 (p, J=7.0 Hz, 2H), 1.44 (s, 6H).

LCMS; m/z 543.1 (M+H)⁺ (ES⁺); 541.0 (M−H)⁻ (ES⁻).

### Example 174: 2-Isopropoxy-N-((5-(2-methoxypyridin-4-yl)-2,3-dihydro-1H-inden-4-yl)carbamoyl)ethanesulfonamide, sodium salt

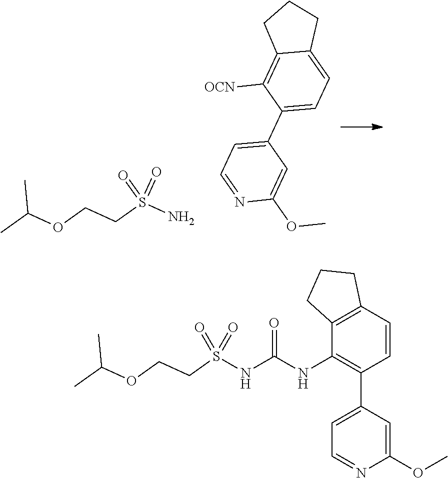

2-Isopropoxyethanesulfonamide (50 mg, 0.299 mmol) was dissolved in dry THF (2 mL). Sodium tert-butoxide (2M in THF) (160 μl, 0.320 mmol) was added and the mixture was stirred at room temperature for 30 minutes. A solution of 4-(4-isocyanato-2,3-dihydro-1H-inden-5-yl)-2-methoxypyridine (Intermediate A48) (80 mg, 0.299 mmol) in THF (1 mL) was added and the mixture was stirred for 2 hours at room temperature. The THF was removed in vacuo. The residue was dissolved in DMSO (2 mL) and then purified by basic prep-HPLC to afford to afford 2-isopropoxy-N-((5-(2-methoxypyridin-4-yl)-2,3-dihydro-1H-inden-4-yl)carbamoyl)ethanesulfonamide as a colourless solid. The solid was dissolved in aq NaOH (0.1 M, 0.74 mL, 1 eq) and the solution was freeze dried overnight to afford the title compound (30 mg, 22%) as a colourless solid.

¹H NMR (DMSO-d6) δ 8.10 (d, J=5.3 Hz, 1H), 7.13-7.02 (m, 3H), 7.00 (d, J=5.3 Hz, 1H), 6.81 (s, 1H), 3.86 (s, 3H), 3.57-3.48 (m, 3H), 3.14-3.06 (m, 2H), 2.90 (t, J=7.4 Hz, 2H), 2.85 (t, J=7.5 Hz, 2H), 1.99 (p, J=7.5 Hz, 2H), 1.07 (d, J=6.1 Hz, 6H).

LCMS; m/z 434.2 (M+H)⁺ (ES⁺); 432.1 (M−H)⁻ (ES⁻).

### Example 175: 2-Isopropoxy-N-((5-(2-methoxypyridin-4-yl)-2,3-dihydrobenzofuran-4-yl)carbamoyl)ethanesulfonamide, sodium salt

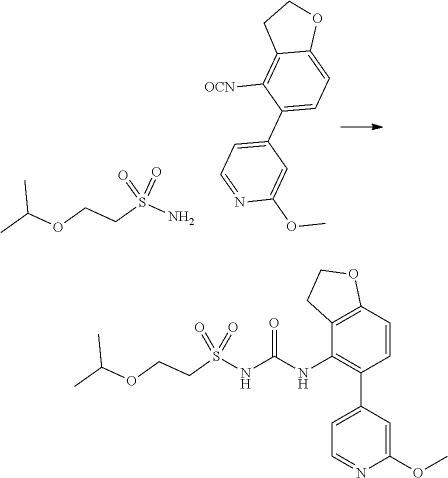

Prepared according to the general procedure of 2-isopropoxy-N-((5-(2-methoxypyridin-4-yl)-2,3-dihydro-1H-inden-4-yl)carbamoyl)ethanesulfonamide, Sodium (Example 174) from 2-isopropoxyethanesulfonamide and 4-(4-isocyanato-2,3-dihydrobenzofuran-5-yl)-2-methoxypyridine (Intermediate A49) and 2-isopropoxyethanesulfonamide to afford the title compound (22 mg, 16%) as a white solid.

¹H NMR (DMSO-d6) δ 8.09 (d, J=5.3 Hz, 1H), 7.20 (s, 1H), 7.03 (d, J=8.2 Hz, 1H), 6.96 (dd, J=5.3, 1.4 Hz, 1H), 6.77 (d, J=1.3 Hz, 1H), 6.62 (d, J=8.2 Hz, 1H), 4.54 (t, J=8.7 Hz, 2H), 3.86 (s, 3H), 3.65-3.47 (m, 3H), 3.20-3.09 (m, 4H), 1.07 (d, J=6.1 Hz, 6H).

LCMS; m/z 436.1 (M+H)⁺ (ES⁺); 434.4 (M−H)⁻ (ES⁻).

### Example 221: 1-Cyclopropyl-N-((5-(2-methoxypyridin-4-yl)-2,3-dihydro-1H-inden-4-yl)carbamoyl)-1H-pyrazole-3-sulfonamide, sodium salt

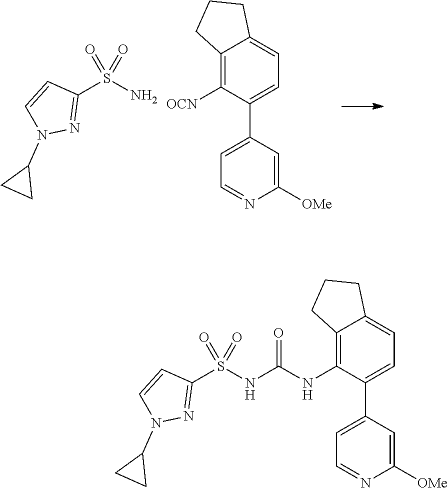

1-Cyclopropyl-1H-pyrazole-3-sulfonamide (Intermediate P29) (516 mg, 2.76 mmol) was dissolved in THF (20 mL), and 2 M sodium tert-butoxide in THF (1.52 mL, 3.04 mmol) was added. After 1 hour, 4-(4-isocyanato-2,3-dihydro-1H-inden-5-yl)-2-methoxypyridine (Intermediate A39) (810 mg, 3.04 mmol) was added and the reaction mixture was stirred at room temperature for 18 hours. Then the reaction mixture was evaporated to dryness, redissolved in DMSO (5 mL) and purified by chromatography on RP Flash C18 (40 g cartridge, 5-50% MeCN/10 mM ammonium bicarbonate) to afford 1-cyclopropyl-N-((5-(2-methoxypyridin-4-yl)-2,3-dihydro-1H-inden-4-yl)carbamoyl)-1H-pyrazole-3-sulfonamide (830 mg, 1.83 mmol). The solid was dissolved with 0.1 M aqueous sodium hydroxide (18.30 mL, 1.83 mmol) and the solution obtained was freeze-dried to afford the title compound (837 mg, 63%) as a white solid.

¹H NMR (DMSO-d6) δ 8.04 (d, J=5.3 Hz, 1H), 7.70 (d, J=2.4 Hz, 1H), 7.34 (s, 1H), 7.07 (d, J=7.7 Hz, 1H), 7.03 (d, J=7.6 Hz, 1H), 6.92 (dd, J=5.2, 1.5 Hz, 1H), 6.75 (s, 1H), 6.28 (d, J=2.3 Hz, 1H), 3.86 (s, 3H), 3.71 (tt, J=7.6, 3.9 Hz, 1H), 2.88 (t, J=7.5 Hz, 2H), 2.73 (t, J=7.4 Hz, 2H), 1.95 (p, J=7.5 Hz, 2H), 1.08-0.91 (m, 4H).

LCMS; m/z 454.3 (M+H)⁺ (ES⁺); 452.1 (M−H)⁻ (ES⁻).

The compounds of examples 176-220 and 222-323 were synthesised by methods analogous to those outlined above.

TABLE 2
¹H NMR and MS data

| Ex | Structure and Name | 1H NMR spectrum | MS | MW |
|---|---|---|---|---|
| 176 | 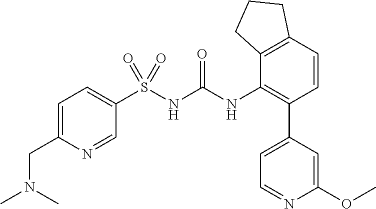 6-((Dimethylamino)methyl)-N-((5-(2- | 1H NMR (DMSO-d6) δ 10.25 (s broad, 1H), 8.88 (d, J = 2.1 Hz, 1H), 8.11- 8.05 (m, 1H), 8.02 (d, J = 5.2 Hz, 1H), 7.52 (d, J = 8.1 Hz, 1H), 7.43 (s, 1H), 7.10 (d, J = 7.7 Hz, 1H), 7.03 (d, J = 7.6 Hz, 1H), 6.81 (d, J = 5.3 Hz, 1H), 6.69 (s, 1H), 4.21 (s, 2H), 3.86 (s, 3H), 2.88 (t, J = 7.4 Hz, 2H), 2.67 (t, J = 7.6 Hz, 2H), 2.62 (s, 6H), 1.94 (p, J = 7.5 Hz, 2H). | m/z 482.2 (M + H)⁺ (ES⁺). | 481.6 |
| | methoxypyridin-4-yl)-2,3-dihydro-1H- | | | |
| | inden-4-yl)carbamoyl)pyridine-3- | | | |
| | sulfonamide | | | |
| 177 | 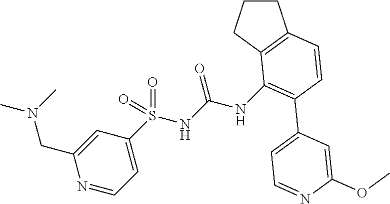 2-((Dimethylamino)methyl)-N-((5-(2- methoxypyridin-4-yl)-2,3-dihydro-1H- | 1H NMR (DMSO-d6) δ 8.46 (dd, J = 5.1, 0.8 Hz, 1H), 8.03 (dd, J = 5.3, 0.7 Hz, 1H), 7.69-7.66 (m, 1H), 7.41 (dd, J = 5.1, 1.6 Hz, 1H), 7.28 (br s, 1H), 7.06 (d, J = 7.7 Hz, 1H), 7.01 (d, J = 7.6 Hz, 1H), 6.89 (dd, J = 5.3, 1.5 Hz, 1H), 6.73 (d, J = 1.2 Hz, 1H), 3.85 (s, 3H), 3.52 (s, 2H), 2.86 (t, J = 7.4 Hz, 2H), 2.70 (t, J = 7.4 Hz, 2H), 2.19 (s, 6H), 1.93 (p, J = 7.5 Hz, 2H). | m/z 482.1 (M + H)⁺ (ES⁺); 480.1 (M − H)⁻ (ES⁻). | 481.6 |
| | inden-4-yl)carbamoyl)pyridine-4- | | | |
| | sulfonamide, sodium salt | | | |
| 178 | 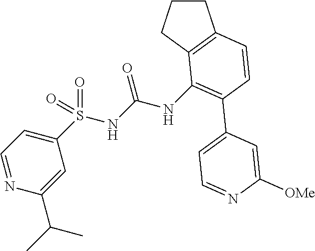 [SMILES: COc1cc(-c2ccc3c(c2NC(=O)NS(=O)(=O)c2ccnc(C(C)C)c2)CCC3)ccn1] | 1H NMR (DMSO-d6) δ 8.48 (dd, J = 5.0, 0.8 Hz, 1H), 8.02 (d, J = 5.4 Hz, 1H), 7.48 (s, 1H), 7.36 (dd, J = 5.0, 1.6 Hz, 1H), 7.31 (br s, 1H), 7.07 (d, J = 7.7 Hz, 1H), 7.02 (d, J = 7.6 Hz, 1H), 6.89 (dd, J = 5.3, 1.4 Hz, 1H), 6.74 (s, 1H), 3.86 (s, 3H), 3.03 (sept, J = 6.9 Hz, 1H), 2.87 (t, J = 7.4 Hz, 2H), 2.70 (t, J = 7.5 Hz, 2H), 1.93 (p, J = 7.5 Hz, 2H), 1.23 (d, J = 6.9 Hz, 6H). | m/z 467.3 (M + H)⁺ (ES⁺); 465.2 (M − H)⁻ (ES⁻). | 466.6 |
| | 2-Isopropyl-N-((5-(2-methoxypyridin-4- | | | |
| | yl)-2,3-dihydro-1H-inden-4- | | | |
| | yl)carbamoyl)pyridine-4-sulfonamide, | | | |
| | sodium salt | | | |
| 179 | 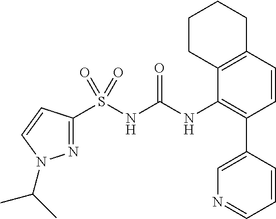 [SMILES: CC(C)n1ccc(S(=O)(=O)NC(=O)Nc2c(-c3cccnc3)ccc3c2CCCC3)n1] | ¹H NMR (DMSO-d6) δ 10.9 (br s, 1H), 8.50 (dd, J = 4.8, 1.7 Hz, 1H), 8.45 (d, J = 2.3 Hz, 1H), 7.94 (br s, 1H), 7.77 (br s, 1H), 7.64 (d, J = 7.9 Hz, 1H), 7.35 (ddd, J = 7.8, 4.9, 0.9 Hz, 1H), 7.12-7.07 (m, 2H), 6.54 (br s, 1H), 4.60 (app p, J = 6.7 Hz, 1H), 2.75 (br s, 2H), 2.47 (br s, 2H), 1.68 (br s, 4H), 1.44 (d, J = 6.7 Hz, 6H). | m/z 440.4 (M + H)⁺ (ES⁺). | 439.5 |
| | 1-Isopropyl-N-((2-(pyridin-3-yl)-5,6,7,8- | | | |
| | tetrahydronaphthalen-1-yl)carbamoyl)- | | | |
| | 1H-pyrazole-3-sulfonamide | | | |
| 180 | 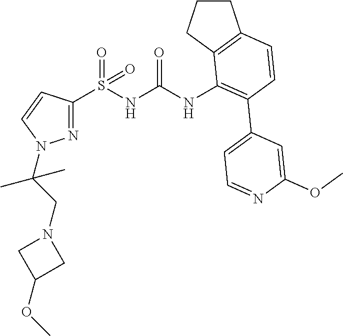 [SMILES: COc1cc(-c2ccc3c(c2NC(=O)NS(=O)(=O)c2ccn(C(C)(C)CN4CC(OC)C4)n2)CCC3)ccn1] | 1H NMR (DMSO-d6) δ 10.87 (br s, 1H), 8.15 (d, J = 5.3 Hz, 1H), 7.95-7.86 (m, 2H), 7.21 (d, J = 7.2 Hz, 1H), 7.11 (d, J = 7.6 Hz, 1H), 6.91-6.87 (m, 1H), 6.73 (br s, 1H), 6.62 (br s, 1H), 3.89 (s, 3H), 3.75 (p, J = 5.8 Hz, 1H), 3.19 (t, J = 6.8 Hz, 2H), 3.09 (s, 3H), 2.91 (t, J = 7.3 Hz, 2H), 2.75 (s, 2H), 2.72-2.62 (m, 4H), 1.98 (p, J = 7.4 Hz, 2H), 1.49 (s, 6H). | m/z 555.2 (M + H)+ (ES+) | 554.66 |
| | 1-(1-(3-Methoxyazetidin-1-yl)-2- | | | |
| | methylpropan-2-yl)-N-((5-(2- | | | |
| | methoxypyridin-4-yl)-2,3-dihydro-1H- | | | |
| | inden-4-yl)carbamoyl)-1H-pyrazole-3- | | | |
| | sulfonamide | | | |
| 181 | 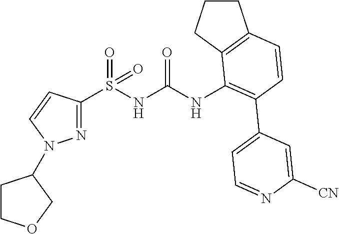 [SMILES: N#Cc1cc(-c2ccc3c(c2NC(=O)NS(=O)(=O)c2ccn(C4CCOC4)n2)CCC3)ccn1] N-((5-(2-Cyanopyridin-4-yl)-2,3-dihydro- 1H-inden-4-yl)carbamoyl)-1- | 1H NMR (DMSO-d6) δ 8.57 (d, J = 5.1 Hz, 1H), 7.90 (d, J = 1.6 Hz, 1H), 7.66 (d, J = 2.3 Hz, 1H), 7.63 (br s, 1H), 7.61 (dd, J = 5.1, 1.8 Hz, 1H), 7.12 (s, 2H), 6.25 (d, J = 2.4 Hz, 1H), 5.00 (ddt, J = 7.9, 6.1, 3.8 Hz, 1H), 3.99- 3.91 (m, 2H), 3.86 (dd, J = 9.4, 3.7 Hz, 1H), 3.79 (td, J = 8.4, 5.6 Hz, 1H), 2.90 (t, J = 7.4 Hz, 2H), 2.77 (t, J = 7.5 Hz, 2H), 2.39-2.33 (m, 1H), 2.22 (m,, 1H), 1.97 (p, J = 7.5 Hz, 2H). | m/z 479.3 (M + H)+ (ES+) | 478.52 |
| | (tetrahydrofuran-3-yl)-1H-pyrazole-3- | | | |
| | sulfonamide, sodium salt | | | |
| 182 | 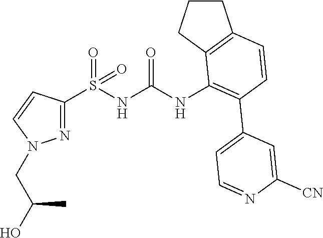 [SMILES: C[C@@H](O)Cn1ccc(S(=O)(=O)NC(=O)Nc2c(-c3ccnc(C#N)c3)ccc3c2CCC3)n1] | 1H NMR (DMSO-d6) δ 8.56 (d, J = 5.1 Hz, 1H), 7.89 (d, J = 1.6 Hz, 1H), 7.64 (br s, 1H), 7.60 (dd, J = 5.1, 1.7 Hz, 1H), 7.57 (d, J = 2.3 Hz, 1H), 7.11 (s, 2H), 6.23 (d, J = 2.2 Hz, 1H), 4.90 (s, 1H), 4.04- 3.90 (m, 3H), 2.90 (t, J = 7.4 Hz, 2H), 2.78 (t, J = 7.5 Hz, 2H), 1.98 (p, J = 7.6 Hz, 2H), 1.01 (d, J = 5.9 Hz, 3H). | m/z 467.3 (M + H)+ (ES+) | 466.51 |
| | (R)-N-((5-(2-Cyanopyridin-4-yl)-2,3- | | | |
| | dihydro-1H-inden-4-yl)carbamoyl)-1-(2- | | | |
| | hydroxypropyl)-1H-pyrazole-3- | | | |
| | sulfonamide, sodium salt | | | |
| 183 | 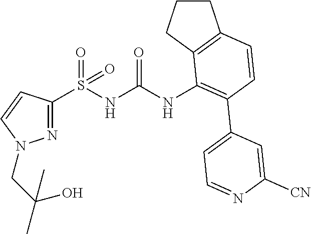 [SMILES: CC(C)(O)Cn1ccc(S(=O)(=O)NC(=O)Nc2c(-c3ccnc(C#N)c3)ccc3c2CCC3)n1] | 1H NMR (DMSO-d6) δ 8.58 (d, J = 5.1 Hz, 1H), 7.90 (d, J = 1.6 Hz, 1H), 7.61 (dd, J = 5.1, 1.7 Hz, 1H), 7.58 (br s, 1H), 7.55 (d, J = 2.3 Hz, 1H), 7.11 (s, 2H), 6.24 (d, J = 2.2 Hz, 1H), 4.68 (s, 1H), 3.99 (s, 2H), 2.89 (t, J = 7.5 Hz, 2H), 2.77 (t, J = 7.4 Hz, 2H), 1.97 (p, J = 7.5 Hz, 2H), 1.03 (s, 6H). | m/z 481.3 (M + H)+ (ES+) | 480.53 |
| | N-((5-(2-Cyanopyridin-4-yl)-2,3-dihydro- | | | |
| | 1H-inden-4-yl)carbamoyl)-1-(2-hydroxy- | | | |
| | 2-methylpropyl)-1H-pyrazole-3- | | | |
| | sulfonamide, sodium salt | | | |
| 184 | 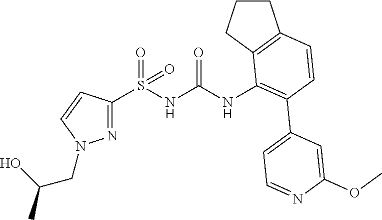 [SMILES: COc1cc(-c2ccc3c(c2NC(=O)NS(=O)(=O)c2ccn(C[C@@H](C)O)n2)CCC3)ccn1] (R)-1-(2-Hydroxypropyl)-N-((5-(2- methoxypyridin-4-yl)-2,3-dihydro-1H- | 1H NMR (DMSO-d6) δ 8.11 (d, J = 5.3 Hz, 1H), 7.80-7.64 (m, 2H), 7.16 (s, 1H), 7.09 (d, J = 7.7 Hz, 1H), 6.89 (d, J = 5.3 Hz, 1H), 6.73 (s, 1H), 6.55- 6.43 (m, 1H), 4.99 (d, J = 5.0 Hz, 1H), 4.09-4.01 (m, 2H), 3.97 (p, J = 5.9 Hz, 1H), 3.88 (s, 3H), 2.90 (t, J = 7.4 Hz, 2H), 2.69 (t, J = 7.2 Hz, 2H), 1.98 (p, J = 7.5 Hz, 2H), 1.04 (d, J = 6.2 Hz, 3H). One exchangeable proton not observed. | m/z 472.3 (M + H)+ (ES+) | 471.53 |
| | inden-4-yl)carbamoyl)-1H-pyrazole-3- | | | |
| | sulfonamide | | | |
| 185 | 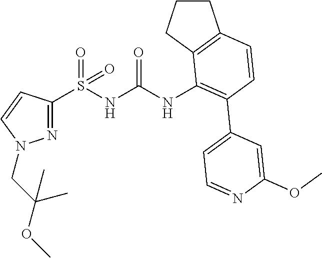 [SMILES: COc1cc(-c2ccc3c(c2NC(=O)NS(=O)(=O)c2ccn(CC(C)(C)OC)n2)CCC3)ccn1] | 1H NMR (DMSO-d6) δ 8.11 (d, J = 5.2 Hz, 1H), 7.74 (s, 1H), 7.67 (s, 1H), 7.17 (d, J = 7.7 Hz, 1H), 7.09 (d, J = 7.6 Hz, 1H), 6.88 (dd, J = 5.3, 1.5 Hz, 1H), 6.73 (s, 1H), 6.53 (s, 1H), 4.17 (s, 2H), 3.88 (s, 3H), 3.16 (s, 3H), 2.90 (t, J = 7.4 Hz, 2H), 2.66 (t, J = 7.5 Hz, 2H), 1.96 (p, J = 7.5 Hz, 2H), 1.06 (s, 6H). One exchangeable proton not observed. | m/z 500.3 (M + H)+ (ES+) | 499.58 |
| | 1-(2-Methoxy-2-methylpropyl)-N-((5-(2- | | | |
| | methoxypyridin-4-yl)-2,3-dihydro-1H- | | | |
| | inden-4-yl)carbamoyl)-1H-pyrazole-3- | | | |
| | sulfonamide | | | |
| 186 | 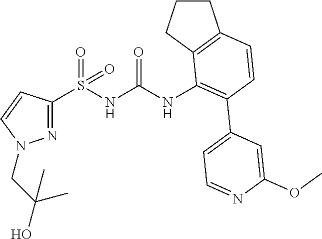 [SMILES: COc1cc(-c2ccc3c(c2NC(=O)NS(=O)(=O)c2ccn(CC(C)(C)O)n2)CCC3)ccn1] | 1H NMR (DMSO-d6) δ 8.11 (d, J = 5.3 Hz, 1H), 7.84-7.61 (m, 2H), 7.16 (d, J = 7.7 Hz, 1H), 7.08 (d, J = 7.6 Hz, 1H), 6.89 (dd, J = 5.3, 1.5 Hz, 1H), 6.73 (s, 1H), 6.51 (s, 1H), 4.76 (s, 1H), 4.06 (s, 2H), 3.88 (s, 3H), 2.90 (t, J = 7.4 Hz, 2H), 2.67 (t, J = 7.5 Hz, 2H), 1.96 (p, J = 7.5 Hz, 2H), 1.05 (s, 6H). One exchangeable proton not observed sulfonamide | m/z 486.3 (M + H)+ (ES+) | 485.56 |
| | 1-(2-Hydroxy-2-methylpropyl)-N-((5-(2- | | | |
| | methoxypyridin-4-yl)-2,3-dihydro-1H- | | | |
| | inden-4-yl)carbamoyl)-1H-pyrazole-3- | | | |
| | sulfonamide | | | |
| 187 | 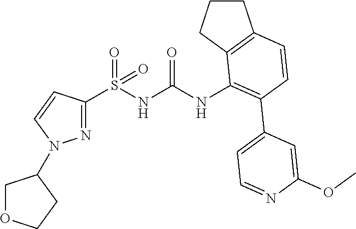 [SMILES: COc1cc(-c2ccc3c(c2NC(=O)NS(=O)(=O)c2ccn(C4CCOC4)n2)CCC3)ccn1] N-((5-(2-Methoxypyridin-4-yl)-2,3- dihydro-1H-inden-4-yl)carbamoyl)-1- (tetrahydrofuran-3-yl)-1H-pyrazole-3- | 1H NMR (DMSO-d6) δ 8.10 (d, J = 5.3 Hz, 1H), 7.87 (s, 1H), 7.76 (s, 1H), 7.17 (d, J = 7.7 Hz, 1H), 7.09 (d, J = 7.6 Hz, 1H), 6.88 (dd, J = 5.3, 1.5 Hz, 1H), 6.73 (s, 1H), 6.55 (s, 1H), 5.13-5.06 (m, 1H), 4.00-3.95 (m, 2H), 3.91- 3.86 (m, 4H), 3.82 (td, J = 8.4, 5.6 Hz, 1H), 2.90 (t, J = 7.4 Hz, 2H), 2.66 (t, J = 7.4 Hz, 2H), 2.49-2.35 (m, 1H), 2.29-2.19 (m, 1H), 1.97 (p, J = 7.5 Hz, 2H). One exchangeable proton not observed. | m/z 484.2 (M + H)+ (ES+) | 483.54 |
| | sulfonamide | | | |
| 188 | 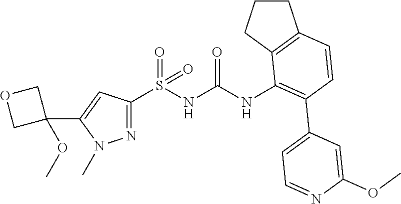 [SMILES: COc1cc(-c2ccc3c(c2NC(=O)NS(=O)(=O)c2cc(C4(OC)COC4)n(C)n2)CCC3)ccn1] 5-(3-Methoxyoxetan-3-yl)-N-((5-(2- | 1H NMR (DMSO-d6) δ 10.90 (s, 1H), 8.14 (d, J = 5.2 Hz, 1H), 7.82 (s, 1H), 7.19 (d, J = 7.7 Hz, 1H), 7.11 (d, J = 7.7 Hz, 1H), 6.96 (s, 1H), 6.90 (dd, J = 5.3, 1.5 Hz, 1H), 6.74- 6.72 (m, 1H), 4.87 (d, J = 7.3 Hz, 2H), 4.80 (d, J = 7.3 Hz, 2H), 3.73 (s, 3H), 3.32 (s, 3H), 2.98 (s, 3H), 2.90 (t, J = 7.4 Hz, 2H), 2.65 (t, J = 7.2 Hz, 2H), 1.96 (p, J = 7.5 Hz, 2H). | m/z 514.3 (M + H)+ (ES+) | 513.57 |
| | methoxypyridin-4-yl)-2,3-dihydro-1H- | | | |
| | inden-4-yl)carbamoyl)-1-methyl-1H- | | | |
| | pyrazole-3-sulfonamide | | | |
| 189 | 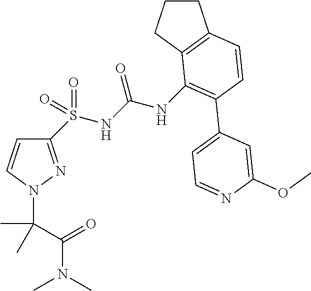 [SMILES: COc1cc(-c2ccc3c(c2NC(=O)NS(=O)(=O)c2ccn(C(C)(C)C(=O)N(C)C)n2)CCC3)ccn1] | ¹H NMR (DMSO-d6) δ 11.03 (s, 1H), 8.14 (d, J = 5.3 Hz, 1H), 7.99 (s, 1H), 7.86 (s, 1H), 7.19 (d, J = 7.7 Hz, 1H), 7.10 (d, J = 7.6 Hz, 1H), 6.90 (dd, J = 5.3, 1.5 Hz, 1H), 6.73 (s, 1H), 6.68 (s, 1H), 3.89 (s, 3H), 2.91 (t, J = 7.4 Hz, 2H), 2.78 (s, 3H), 2.65 (t, J = 7.3 Hz, 2H), 2.22 (s, 3H), 1.97 (p, J = 7.5 Hz, 2H), 1.70 (s, 6H). | m/z 527.3 (M + H)⁺ (ES⁺); 525.3 (M − H)⁻ (ES⁻). | 526.6 |
| | 2-(3-(N-((5-(2-Methoxypyridin-4-yl)-2,3- | | | |
| | dihydro-1H-inden-4- | | | |
| | yl)carbamoyl)sulfamoyl)-1H-pyrazol-1- | | | |
| | yl)-N,N,2-trimethylpropanamide | | | |
| 190 | 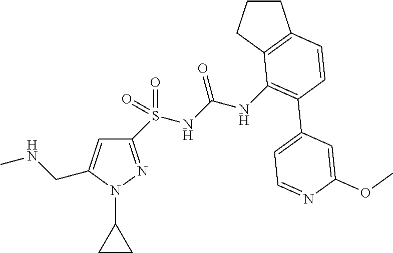 | ¹H NMR (DMSO-d6) δ 8.07-8.04 (m, 1H), 7.36 (s, 1H), 7.07 (d, J = 8.1 Hz, 1H), 7.04 (d, J = 7.6 Hz, 1H), 6.96-6.92 (m, 1H), 6.76 (s, 1H), 6.28- 6.24 (m, 1H), 3.86 (s, 3H), 3.78-3.74 (m, 2H), 3.63- 3.57 (m, 1H), 2.88 (t, J = 7.4 Hz, 2H), 2.72 (t, J = 7.4 Hz, 2H), 2.30 (d, J = 6.2 Hz, 3H), 2.08-1.99 (m, 1H), 1.94 (p, J = 7.5 Hz, 2H), 1.10-1.04 (m, 2H), 0.97-0.92 (m, 2H). | m/z 497.2 (M + H)⁺ (ES⁺); 495.0 (M − H)⁻ (ES⁺). | 496.6 |
| | 1-Cyclopropyl-N-((5-(2-methoxypyridin- | | | |
| | 4-yl)-2,3-dihydro-1H-inden-4- | | | |
| | yl)carbamoyl)-5-((methylamino)methyl)- | | | |
| | 1H-pyrazole-3-sulfonamide, sodium salt | | | |
| 191 | 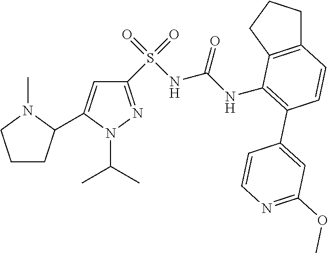 [SMILES: COc1cc(-c2ccc3c(c2NC(=O)NS(=O)(=O)c2cc(C4CCCN4C)n(C(C)C)n2)CCC3)ccn1] 1-Isopropyl-N-((5-(2-methoxypyridin-4- | ¹H NMR (DMSO-d6) δ 8.06 (d, J = 5.3 Hz, 1H), 7.45 (s, 1H), 7.08 (d, J = 7.7 Hz, 1H), 7.04 (d, J = 7.6 Hz, 1H), 6.95 (d, J = 5.3 Hz, 1H), 6.78 (s, 1H), 6.24 (s, 1H), 4.75 (sept, J = 6.7 Hz, 1H), 3.87 (s, 3H), 3.14-3.08 (m, 1H), 2.86 (t, J = 7.4 Hz, 2H), 2.63 (t, J = 7.5 Hz, 2H), 2.25-2.19 (m, 2H), 2.17 (s, 3H), 1.89 (p, J = 7.4 Hz, 2H), 1.85-1.58 (m, 4H), 1.38 (d, J = 6.6 Hz, 3H), 1.34 (d, J = 6.5 Hz, 3H). | m/z 539.3 (M + H)⁺ (ES⁺); 537.1 (M − H)⁻ (ES⁻). | 538.7 |
| | yl)-2,3-dihydro-1H-inden-4- | | | |
| | yl)carbamoyl)-5-(1-methylpyrrolidin-2- | | | |
| | yl)-1H-pyrazole-3-sulfonamide, sodium | | | |
| | salt | | | |
| 192 | 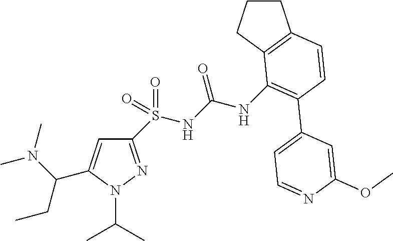 5-(1-(Dimethylamino)propyl)-1-isopropyl- N-((5-(2-methoxypyridin-4-yl)-2,3- dihydro-1H-inden-4-yl)carbamoyl)-1H- pyrazole-3-sulfonamide | ¹H NMR (DMSO-d6) δ 10.84 (s, 1H), 8.15 (dd, J = 5.2, 0.6 Hz, 1H), 7.90 (s, 1H), 7.22 (d, J = 7.7 Hz, 1H), 7.12 (d, J = 7.6 Hz, 1H), 6.88 (dd, J = 5.3, 1.4 Hz, 1H), 6.72 (d, J = 1.4 Hz, 1H), 6.57 (s, 1H), 4.86 (sept, J = 6.6 Hz, 1H), 3.88 (s, 3H), 3.75-3.67 (m, 1H), 2.90 (t, J = 7.4 Hz, 2H), 2.56 (t, J = 7.7 Hz, 2H), 2.14 (s, 6H), 1.92 (p, J = 7.4 Hz, 2H), 1.89- 1.80 (m, 1H), 1.71-1.60 (m, 1H), 1.41 (d, J = 6.5 Hz, 3H), 1.34 (d, J = 6.5 Hz, 3H), 0.77 (t, J = 7.3 Hz, 3H). | m/z 541.3 (M + H)⁺ (ES⁺); 539.3 (M − H)⁻ (ES⁻). | 540.7 |
| 193 | 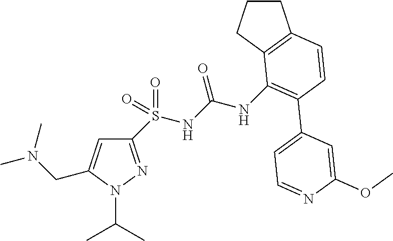 5-((Dimethylamino)methyl)-1-isopropyl- | ¹H NMR (DMSO-d6) δ 10.85 (s, 1H), 8.14 (d, J = 5.3 Hz, 1H), 7.91 (s, 1H), 7.22 (d, J = 7.6 Hz, 1H), 7.12 (d, J = 7.6 Hz, 1H), 6.88 (dd, J = 5.3, 1.5 Hz, 1H), 6.73-6.71 (m, 1H), 6.55 (s, 1H), 4.81 (sept, J = 6.6 Hz, 1H), 3.88 (s, 3H), 3.50 (s, 2H), 2.90 (t, J = 7.5 Hz, 2H), 2.59 (t, J = 7.4 Hz, 2H), 2.16 (s, 6H), 1.95 (p, J = 7.6 Hz, 2H), 1.38 (d, J = 6.6 Hz, 6H). | m/z 513.3 (M + H)⁺ (ES⁺); 511.3 (M − H)⁻ (ES⁻). | 512.6 |
| | N-((5-(2-methoxypyridin-4-yl)-2,3- | | | |
| | dihydro-1H-inden-4-yl)carbamoyl)-1H- | | | |
| | pyrazole-3-sulfonamide | | | |
| 194 | 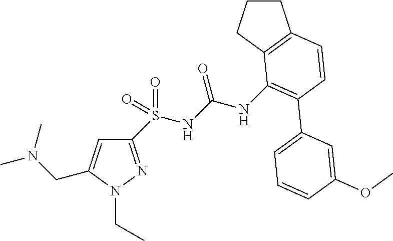 | ¹H NMR (DMSO-d6) δ 10.74 (s, 1H), 7.79 (s, 1H), 7.33-7.28 (m, 1H), 7.19 (d, J = 7.7 Hz, 1H), 7.09 (d, J = 7.6 Hz, 1H), 6.95- 6.91 (m, 1H), 6.87-6.79 (m, 2H), 6.61 (s, 1H), 4.23 (q, J = 7.2 Hz, 2H), 3.70 (s, 3H), 3.51 (s, 2H), 2.90 (t, J = 7.4 Hz, 2H), 2.58 (t, J = 7.4 Hz, 2H), 2.18 (s, 6H), 1.95 (p, J = 7.5 Hz, 2H), 1.36 (t, J = 7.2 Hz, 3H). | m/z 498.4 (M + H)⁺ (ES⁺); 496.1 (M − H)⁻ (ES⁻). | 497.6 |
| | 5-((Dimethylamino)methyl)-1-ethyl-N- | | | |
| | ((5-(3-methoxyphenyl)-2,3-dihydro-1H- | | | |
| | inden-4-yl)carbamoyl)-1H-pyrazole-3- | | | |
| | sulfonamide | | | |
| 195 | 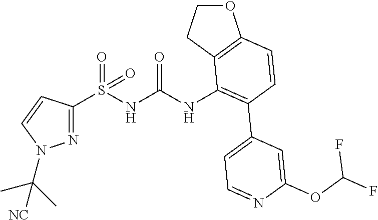 [SMILES: CC(C)(C#N)n1ccc(S(=O)(=O)NC(=O)Nc2c(-c3ccnc(OC(F)F)c3)ccc3c2CCO3)n1] 1-(2-Cyanopropan-2-yl)-N-((5-(2- | 1H NMR (DMSO-d6) δ 8.15 (d, J = 5.3 Hz, 1H), 7.95 (d, J = 2.8 Hz, 1H), 7.82 (d, J = 73.2 Hz, 1H), 7.62 (br s, 1H), 7.20 (dd, J = 5.3, 1.5 Hz, 1H), 7.08 (d, J = 8.2 Hz, 1H), 6.99 (d, J = 1.5 Hz, 1H), 6.65 (d, J = 8.2 Hz, 1H), 6.45 (d, J = 2.8 Hz, 1H), 4.51 (t, J = 8.2 Hz, 2H), 3.08 (t, J = 8.2 Hz, 2H), 1.96 (s, 6H). One exchangeable proton not observed. | m/z 519.1 (M + H)+ (ES+) | 518.49 |
| | (difluoromethoxy)pyridin-4-yl)-2,3- | | | |
| | dihydrobenzofuran-4-yl)carbamoyl)-1H- | | | |
| | pyrazole-3-sulfonamide | | | |
| 196 | 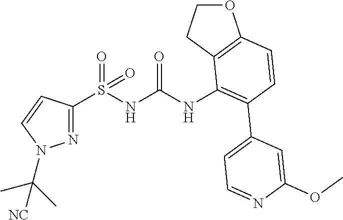 [SMILES: COc1cc(-c2ccc3c(c2NC(=O)NS(=O)(=O)c2ccn(C(C)(C)C#N)n2)CCO3)ccn1] | 1H NMR (DMSO-d6) δ 11.17 (br s, 1H), 8.16 (d, J = 2.8 Hz, 1H), 8.12 (d, J = 5.3 Hz, 1H), 7.95 (br s, 1H), 7.09 (d, J = 8.2 Hz, 1H), 6.87 (dd, J = 5.3, 1.4 Hz, 1H), 6.77-6.66 (m, 3H), 4.53 (t, J = 8.7 Hz, 2H), 3.88 (s, 3H), 3.00 (t, J = 8.7 Hz, 2H), 2.00 (s, 6H). | m/z 483.2 (M + H)+ (ES+) | 482.51 |
| | 1-(2-Cyanopropan-2-yl)-N-((5-(2- | | | |
| | methoxypyridin-4-yl)-2,3- | | | |
| | dihydrobenzofuran-4-yl)carbamoyl)-1H- | | | |
| | pyrazole-3-sulfonamide | | | |
| 197 | 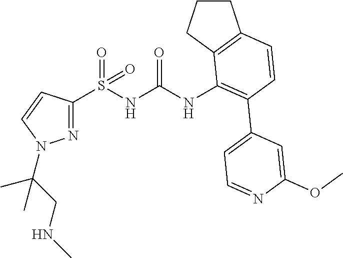 [SMILES: CNCC(C)(C)n1ccc(S(=O)(=O)NC(=O)Nc2c(-c3ccnc(OC)c3)ccc3c2CCC3)n1] N-((5-(2-Methoxypyridin-4-yl)-2,3- | 1H NMR (DMSO-d6) δ 8.05 (dd, J = 5.3, 0.7 Hz, 1H), 7.73 (d, J = 2 .4 Hz, 1H), 7.37 (s, 1H), 7.07 (d, J = 7.7 Hz, 1H), 7.03 (d, J = 7.7 Hz, 1H), 6.92 (dd, J = 5.3, 1.5 Hz, 1H), 6.77- 6.75 (m, 1H), 6.32 (d, J = 2.3 Hz, 1H), 3.87 (s, 3H), 2.87 (t, J = 7.4 Hz, 2H), 2.74 (d, J = 7.9 Hz, 2H), 2.70 (t, J = 7.5 Hz, 2H), 2.17 (d, J = 6.1 Hz, 3H), 1.93 (p, J = 7.5 Hz, 2H), 1.48 (s, 6H), 1.35-1.24 (m, 1H). | m/z 499.2 (M + H)+ (ES+) | 498.60 |
| | dihydro-1H-inden-4-yl)carbamoyl)-1-(2- | | | |
| | methyl-1-(methylamino)propan-2-yl)-1H- | | | |
| | pyrazole-3-sulfonamide, sodium salt | | | |
| 198 | 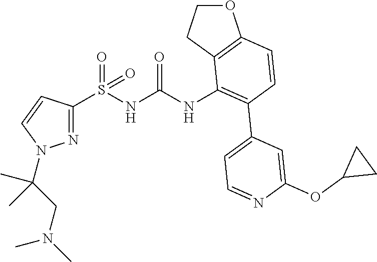 [SMILES: CN(C)CC(C)(C)n1ccc(S(=O)(=O)NC(=O)Nc2c(-c3ccnc(OC4CC4)c3)ccc3c2CCO3)n1] | 1H NMR (DMSO-d6) δ 8.12-8.06 (m, 1H), 7.72- 7.65 (m, 1H), 7.47-7.37 (m, 1H), 7.02 (d, J = 8.2 Hz, 1H), 6.96-6.93 (m, 1H), 6.83 (s, 1H), 6.61 (d, J = 8.4, 3.4 Hz, 1H), 6.35- 6.30 (m, 1H), 4.49 (t, J = 8.8 Hz, 2H), 4.22-4.17 (m, 1H), 3.04 (t, J = 8.8 Hz, 2H), 1.92 (s, 6H), 1.48 (s, 6H), 0.81-0.64 (m, 4H). One CH2 obscured by DMSO peak. | m/z 541.2 (M + H)+ (ES+) | 540.63 |
| | N-((5-(2-Cyclopropoxypyridin-4-yl)-2,3- | | | |
| | dihydrobenzofuran-4-yl)carbamoyl)-1-(1- | | | |
| | (dimethylamino)-2-methylpropan-2-yl)- | | | |
| | 1H-pyrazole-3-sulfonamide, sodium salt | | | |
| 199 | 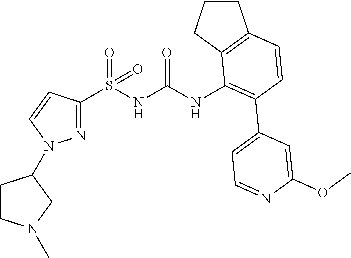 [SMILES: COc1cc(-c2ccc3c(c2NC(=O)NS(=O)(=O)c2ccn(C4CCN(C)C4)n2)CCC3)ccn1] | 1H NMR (DMSO-d6) δ 8.03 (d, J = 5.2 Hz, 1H), 7.74-7.70 (m, 1H), 7.36 (s, 1H), 7.07 (d, J = 7.7 Hz, 1H), 7.03 (d, J = 7.6 Hz, 1H), 6.91 (d, J = 5.9 Hz, 1H), 6.75 (s, 1H), 6.32- 6.29 (m, 1H), 4.92-4.85 (m, 1H), 3.86 (s, 3H), 2.88 (t, J = 7.4 Hz, 2H), 2.83-2.67 (m, 5H), 2.45- 2.39 (m, 1H), 2.39-2.30 (m, 1H), 2.28 (s, 3H), 2.08-1.99 (m, 1H), 1.94 (p, J = 7.6 Hz, 2H). | m/z 497.1 (M + H)+ (ES+) | 496.58 |
| | N-((5-(2-Methoxypyridin-4-yl)-2,3- | | | |
| | dihydro-1H-inden-4-yl)carbamoyl)-1-(1- | | | |
| | methylpyrrolidin-3-yl)-1H-pyrazole-3- | | | |
| | sulfonamide, sodium salt | | | |
| 200 | 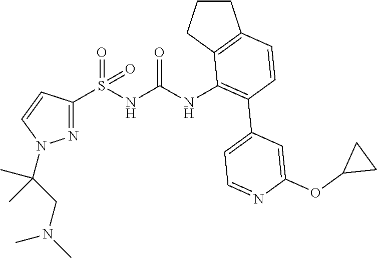 [SMILES: CN(C)CC(C)(C)n1ccc(S(=O)(=O)NC(=O)Nc2c(-c3ccnc(OC4CC4)c3)ccc3c2CCC3)n1] | 1H NMR (DMSO-d6) δ 8.09 (d, J = 5.3 Hz, 1H), 7.70 (d, J = 2.4 Hz, 1H), 7.37 (s, 1H), 7.08 (d, J = 7.7 Hz, 1H), 7.04 (d, J = 7.6 Hz, 1H), 6.97 (dd, J = 5.2, 1.4 Hz, 1H), 6.86 (d, J = 1.4 Hz, 1H), 6.32 (d, J = 2.3 Hz, 1H), 4.20 (tt, J = 6.2, 3.1 Hz, 1H), 2.87 (t, J = 7.4 Hz, 2H), 2.71 (t, J = 7.4 Hz, 2H), 2.53 (s, 2H), 1.98-1.89 (m, 8H), 1.49 (s, 6H), 0.81-0.74 (m, 2H), 0.70-0.64 (m, 2H). | m/z 539.1 (M + H)+ (ES+) | 538.66 |
| | N-((5-(2-Cyclopropoxypyridin-4-yl)-2,3- | | | |
| | dihydro-1H-inden-4-yl)carbamoyl)-1-(1- | | | |
| | (dimethylamino)-2-methylpropan-2-yl)- | | | |
| | 1H-pyrazole-3-sulfonamide, sodium salt | | | |
| 201 | 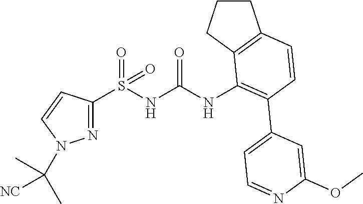 [SMILES: COc1cc(-c2ccc3c(c2NC(=O)NS(=O)(=O)c2ccn(C(C)(C)C#N)n2)CCC3)ccn1] 1-(2-Cyanopropan-2-yl)-N-((5-(2- | 1H NMR (DMSO-d6) δ 8.07 (d, J = 5.3, 1H), 7.97 (d, J = 2.5 Hz, 1H), 7.38 (br s, 1H), 7.08 (d, J = 7.6 Hz, 1H), 7.04 (d, J = 7.6 Hz, 1H), 6.94 (dd, J = 5.3, 1.4 Hz, 1H), 6.77 (d, J = 1.4 Hz, 1H), 6.45 (d, J = 2.5 Hz, 1H), 3.87 (s, 3H), 2.88 (t, J = 7.5 Hz, 2H), 2.73 (t, J = 7.5 Hz, 2H), 1.97 (s, 6H), 2.73 (p, J = 7.5 Hz, 2H). | m/z 481.3 (M + H)+ (ES+) | 480.54 |
| | methoxypyridin-4-yl)-2,3-dihydro-1H- | | | |
| | inden-4-yl)carbamoyl)-1H-pyrazole-3- | | | |
| | sulfonamide, sodium salt | | | |
| 202 | 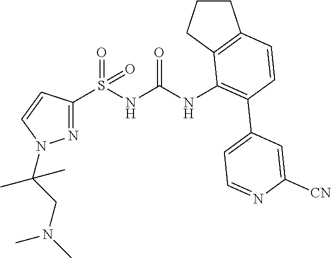 [SMILES: CN(C)CC(C)(C)n1ccc(S(=O)(=O)NC(=O)Nc2c(-c3ccnc(C#N)c3)ccc3c2CCC3)n1] | 1H NMR (DMSO-d6) δ 10.93 (br. s, 1H), 8.70 (dd, J = 5.1, 0.8 Hz, 1H), 8.15 (br. s, 1H), 7.96 (dd, J = 1.8, 0.8 Hz, 1H), 7.89 (d, J = 2.5 Hz, 1H), 7.64 (dd, J = 5.1, 1.8 Hz, 1H), 7.27 (d, J = 7.7 Hz, 1H), 7.21 (d, J = 7.7 Hz, 1H), 6.55 (d, J = 2.4 Hz, 1H), 2.94 (t, J = 7.5 Hz, 2H), 2.70 (t, J = 7.5 Hz, 2H), 2.59 (s, 2H), 2.01 (p, J = 7.6 Hz, 2H), 1.92 (s, 6H), 1.52 (s, 6H). | m/z 508.4 (M + H)+ (ES+) | 507.61 |
| | N-((5-(2-Cyanopyridin-4-yl)-2,3-dihydro- | | | |
| | 1H-inden-4-yl)carbamoyl)-1-(1- | | | |
| | (dimethylamino)-2-methylpropan-2-yl)- | | | |
| | 1H-pyrazole-3-sulfonamide | | | |
| 203 | 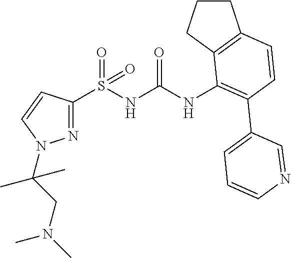 [SMILES: CN(C)CC(C)(C)n1ccc(S(=O)(=O)NC(=O)Nc2c(-c3cccnc3)ccc3c2CCC3)n1] | 1H NMR (DMSO-d6) δ 10.81 (s, 1H), 8.55 (dd, J = 4.8, 1.7 Hz, 1H), 8.49 (dd, J = 2.3, 0.8 Hz, 1H), 7.96 (d, J = 2.5 Hz, 1H), 7.92 (s, 1H), 7.70-7.66 (m, 1H), 7.44-7.40 (m, 1H), 7.23 (d, J = 7.6 Hz, 1H), 7.13 (d, J = 7.6 Hz, 1H), 6.62 (d, J = 2.4 Hz, 1H), 2.92 (t, J = 7.4 Hz, 2H), 2.68-2.61 (m, 2H), 2.59 (s, 2H), 1.98 (p, J = 7.5 Hz, 2H), 1.93 (s, 6H), 1.53 (s, 6H). | m/z 483.5 (M + H)+ (ES+) | 482.60 |
| | 1-(1-(Dimethylamino)-2-methylpropan-2- | | | |
| | yl)-N-((5-(pyridin-3-yl)-2,3-dihydro-1H- | | | |
| | inden-4-yl)carbamoyl)-1H-pyrazole-3- | | | |
| | sulfonamide | | | |
| 204 | 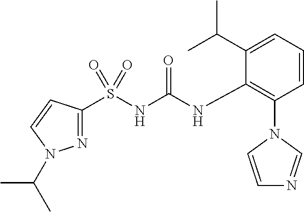 [SMILES: CC(C)c1cccc(-n2ccnc2)c1NC(=O)NS(=O)(=O)c1ccn(C(C)C)n1] | 1H NMR (DMSO-d6) δ 11.25 (s, 1H), 7.99-7.81 (m, 2H), 7.68 (s, 1H), 7.45- 7.34 (m, 2H), 7.26-7.13 (m, 2H), 6.98 (s, 1H), 6.59 (s, 1H), 4.61 (hept, J = 6.8 Hz, 1H), 3.09-2.91 (m, 1H), 1.44 (d, J = 6.7 Hz, 6H), 1.09 (d, J = 6.9 Hz, 6H). | m/z 417.4 (M + H)⁺ (ES⁺); 415.1 (M − H)⁻ (ES⁻) | 416.50 |
| | N-((2-(1H-Imidazol-1-yl)-6- | | | |
| | isopropylphenyl)carbamoyl)-1-isopropyl- | | | |
| | 1H-pyrazole-3-sulfonamide | | | |
| 205 | 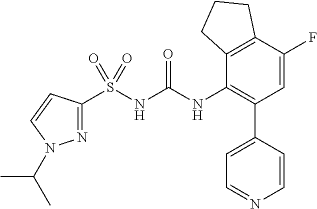 [SMILES: CC(C)n1ccc(S(=O)(=O)NC(=O)Nc2c(-c3ccncc3)cc(F)c3c2CCC3)n1] | 1H NMR (DMSO-d6) δ 10.92 (br s, 1H), 8.62- 8.46 (m, 2H), 7.93 (d, J = 2.3 Hz, 1H), 7.88 (br s, 1H), 7.37-7.25 (m, 2H), 7.03 (d, J = 9.2 Hz, 1H), 6.58 (s, 1H), 4.60 (sept, J = 6.7 Hz, 1H), 2.95 (t, J = 7.4 Hz, 2H), 2.69 (t, J = 7.1 Hz, 2H), 2.04 (p, J = 7.6 Hz, 2H), 1.44 (d, J = 6.7 Hz, 6H). | m/z 444.3 (M + H)⁺ (ES⁺); 442.4 (M − H)⁻ (ES⁻) | 443.49 |
| | N-((7-Fluoro-5-(pyridin-4-yl)-2,3- | | | |
| | dihydro-1H-inden-4-yl)carbamoyl)-1- | | | |
| | isopropyl-1H-pyrazole-3-sulfonamide | | | |
| 206 | 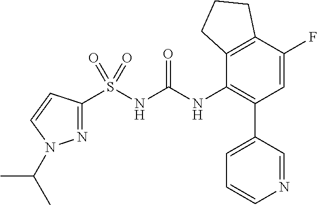 [SMILES: CC(C)n1ccc(S(=O)(=O)NC(=O)Nc2c(-c3cccnc3)cc(F)c3c2CCC3)n1] N-((7-Fluoro-5-(pyridin-3-yl)-2,3- | 1H NMR (DMSO-d6) δ 10.90 (s, 1H), 8.55 (dd, J = 4.8, 1.6 Hz, 1H), 8.51 (d, J = 2.3 Hz, 1H), 7.91 (s, 1H), 7.84 (s, 1H), 7.73- 7.68 (m, 1H), 7.40 (dd, J = 7.9, 4.8 Hz, 1H), 7.02 (d, J = 9.3 Hz, 1H), 6.56 (s, 1H), 4.60 (sept, J = 6.6 Hz, 1H), 2.95 (t, J = 7.4 Hz, 2H), 2.68 (t, J = 7.4 Hz, 2H), 2.04 (p, J = 7.6 Hz, 2H), 1.44 (d, J = 6.7 Hz, 6H). | m/z 444.3 (M + H)⁺ (ES⁺); 442.3 (M − H)⁻ (ES⁻) | 443.49 |
| | dihydro-1H-inden-4-yl)carbamoyl)-1- | | | |
| | isopropyl-1H-pyrazole-3-sulfonamide | | | |
| 207 | 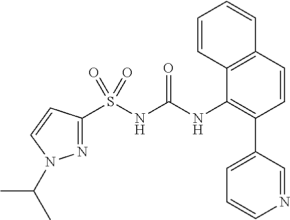 [SMILES: CC(C)n1ccc(S(=O)(=O)NC(=O)Nc2c(-c3cccnc3)ccc3ccccc23)n1] | 1H NMR (DMSO-d6) δ 11.13 (s, 1H), 8.63 (d, J = 2.3 Hz, 1H), 8.57 (dd, J = 4.8, 1.6 Hz, 1H), 8.40 (s, 1H), 8.03-7.97 (m, 2H), 7.95 (s, 1H), 7.90-7.78 (m, 2H), 7.61-7.52 (m, 3H), 7.43 (dd, J = 7.8, 4.8 Hz, 1H), 6.57 (s, 1H), 4.64 (sept, J = 6.7 Hz, 1H), 1.47 (d, J = 6.7 Hz, 6H). | m/z 436.4 (M + H)⁺ (ES⁺); 434.3 (M − H)⁻ (ES⁻) | 435.50 |
| | 1-Isopropyl-N-((2-(pyridin-3- | | | |
| | yl)naphthalen-1-yl)carbamoyl)-1H- | | | |
| | pyrazole-3-sulfonamide | | | |
| 208 | 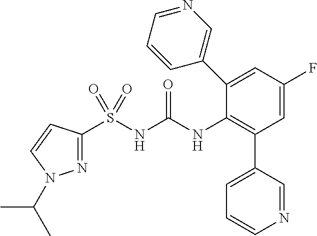 [SMILES: CC(C)n1ccc(S(=O)(=O)NC(=O)Nc2c(-c3cccnc3)cc(F)cc2-c2cccnc2)n1] | 1H NMR (DMSO-d6) δ 10.88 (s, 1H), 8.58 (d, J = 2.3 Hz, 2H), 8.56 (dd, J = 4.8, 1.6 Hz, 2H), 7.90 (s, 1H), 7.84 (s, 1H), 7.82- 7.76 (m, 2H), 7.38 (dd, J = 7.9, 4.8 Hz, 2H), 7.35 (d, J = 8.9 Hz, 2H), 6.24 (s, 1H), 4.57 (sept, J = 6.7 Hz, 1H), 1.42 (d, J = 6.7 Hz, 6H). | m/z 481.3 (M + H)⁺ (ES⁺) | 480.51 |
| | N-((4-Fluoro-2,6-di(pyridin-3- | | | |
| | yl)phenyl)carbamoyl)-1-isopropyl-1H- | | | |
| | pyrazole-3-sulfonamide | | | |
| 209 | 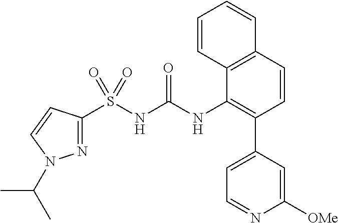 [SMILES: COc1cc(-c2ccc3ccccc3c2NC(=O)NS(=O)(=O)c2ccn(C(C)C)n2)ccn1] | 1H NMR (DMSO-d6) δ 11.15 (s, 1H), 8.38 (s, 1H), 8.15 (d, J = 5.2 Hz, 1H), 8.00 (d, J = 7.9 Hz, 1H), 7.97 (d, J = 8.4 Hz, 1H), 7.93 (s, 1H), 7.86 (d, J = 8.2 Hz, 1H), 7.62-7.52 (m, 2H), 7.50 (d, J = 8.5 Hz, 1H), 7.02 (d, J = 6.2 Hz, 1H), 6.88 (s, 1H), 6.58 (s, 1H), 4.62 (sept, J = 7.0 Hz, 1H), 3.91 (s, 3H), 1.46 (d, J = 6.7 Hz, 6H). | m/z 466.4 (M + H)⁺ (ES⁺); 464.3 (M − H)⁻ (ES⁻) | 465.52 |
| | 1-Isopropyl-N-((2-(2-methoxypyridin-4- | | | |
| | yl)naphthalen-1-yl)carbamoyl)-1H- | | | |
| | pyrazole-3-sulfonamide | | | |
| 210 | 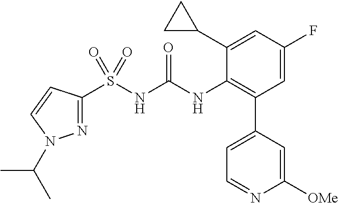 [SMILES: COc1cc(-c2cc(F)cc(C3CC3)c2NC(=O)NS(=O)(=O)c2ccn(C(C)C)n2)ccn1] N-((2-Cyclopropyl-4-fluoro-6-(2- | 1H NMR (DMSO-d6) δ 10.97 (s, 1H), 8.09 (d, J = 5.3 Hz, 1H), 7.95-7.85 (m, 2H), 7.00 (dd, J = 8.9, 3.0 Hz, 1H), 6.91 (d, J = 5.3 Hz, 1H), 6.83 (dd, J = 10.0, 3.0 Hz, 1H), 6.78 (s, 1H), 6.56 (s, 1H), 4.57 (sept, J = 6.7 Hz, 1H), 3.88 (s, 3H), 1.88-1.79 (m, 1H), 1.42 (d, J = 6.7 Hz, 6H), 0.86-0.77 (m, 2H), 0.70-0.59 (m, 2H). | m/z 474.4 (M + H)⁺ (ES⁺); 472.3 (M − H)⁻ (ES⁻) | 473.52 |
| | methoxypyridin-4-yl)phenyl)carbamoyl)- | | | |
| | 1-isopropyl-1H-pyrazole-3-sulfonamide | | | |
| 211 | 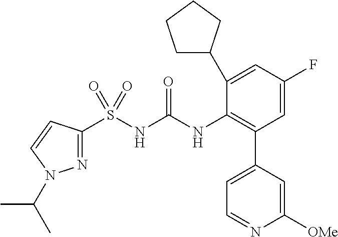 [SMILES: COc1cc(-c2cc(F)cc(C3CCCC3)c2NC(=O)NS(=O)(=O)c2ccn(C(C)C)n2)ccn1] N-((2-Cyclopentyl-4-fluoro-6-(2- | 1H NMR (DMSO-d6) δ 10.94 (s, 1H), 8.09 (d, J = 5.3 Hz, 1H), 7.92 (s, 1H), 7.84 (s, 1H), 7.20 (dd, J = 10.1, 3.0 Hz, 1H), 7.03 (dd, J = 8.9, 3.0 Hz, 1H), 6.91 (dd, J = 5.3, 1.4 Hz, 1H), 6.77 (s, 1H), 6.55 (s, 1H), 4.58 (sept, J = 6.7 Hz, 1H), 3.88 (s, 3H), 3.08-2.98 (m, 1H), 1.95- 1.80 (m, 2H), 1.80-1.69 (m, 2H), 1.59-1.49 (m, 2H), 1.47-1.38 (m, 2H), 1.43 (d, J = 6.7 Hz, 6H). | m/z 502.4 (M + H)⁺ (ES⁺); 500.3 (M − H)⁻ (ES⁻) | 501.57 |
| | methoxypyridin-4-yl)phenyl)carbamoyl)- | | | |
| | 1-isopropyl-1H-pyrazole-3-sulfonamide | | | |
| 212 | 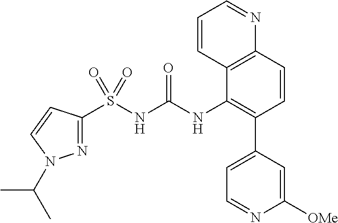 [SMILES: COc1cc(-c2ccc3ncccc3c2NC(=O)NS(=O)(=O)c2ccn(C(C)C)n2)ccn1] 1-Isopropyl-N-((6-(2-methoxypyridin-4- | 1H NMR (DMSO-d6) δ 11.24 (s, 1H), 8.97-8.94 (m, 1H), 8.46 (s, 1H), 8.22 (d, J = 8.4 Hz, 1H), 8.18 (d, J = 5.3 Hz, 1H), 8.05 (d, J = 8.6 Hz, 1H), 7.92 (s, 1H), 7.75 (d, J = 8.8 Hz, 1H), 7.58 (dd, J = 8.6, 4.2 Hz, 1H), 7.03 (d, J = 5.4 Hz, 1H), 6.89 (d, J = 1.2 Hz, 1H), 6.57 (s, 1H), 4.61 (sept, J = 6.7 Hz, 1H), 3.92 (s, 3H), 1.45 (d, J = 6.7 Hz, 6H). | m/z 467.4 (M + H)⁺ (ES⁺); 465.2 (M − H)⁻ (ES⁻) | 466.51 |
| | yl)quinolin-5-yl)carbamoyl)-1H-pyrazole- | | | |
| | 3-sulfonamide | | | |
| 213 | 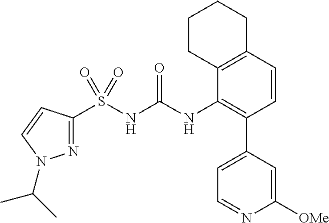 [SMILES: COc1cc(-c2ccc3c(c2NC(=O)NS(=O)(=O)c2ccn(C(C)C)n2)CCCC3)ccn1] | ¹H NMR (DMSO-d6) δ 10.9 (br. s, 1H), 8.07 (d, J = 5.0 Hz, 1H), 7.93 (br. s, 1H), 7.75 (br. s, 1H), 7.11- 7.06 (m, 2H), 6.84 (d, J = 5.0 Hz, 1H), 6.69 (br. s, 1H), 6.58 (br. s, 1H), 4.59 (app. p, J = 6.7 Hz, 1H), 3.87 (s, 3H), 2.75 (s, 2H), 2.46 (br. s, 2H), 1.67 (br. s, 4H), 1.43 (d, J = 6.7 Hz, 6H). | m/z 470.2 (M + H)⁺ (ES⁺) | 469.56 |
| | 1-Isopropyl-N-((2-(2-methoxypyridin-4- | | | |
| | yl)-5,6,7,8-tetrahydronaphthalen-1- | | | |
| | yl)carbamoyl)-1H-pyrazole-3-sulfonamide | | | |
| 214 | 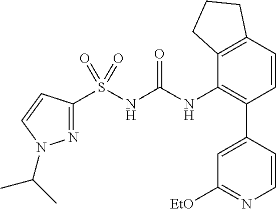 [SMILES: CCOc1cc(-c2ccc3c(c2NC(=O)NS(=O)(=O)c2ccn(C(C)C)n2)CCC3)ccn1] N-((5-(2-Ethoxypyridin-4-yl)-2,3- dihydro-1H-inden-4-yl)carbamoyl)-1- isopropyl-1H-pyrazole-3-sulfonamide | ¹H NMR (DMSO-d₆) δ 10.85 (s, 1H), 8.09 (d, J = 5.3 Hz, 1H), 7.94 (s, 1H), 7.91 (s, 1H), 7.20 (d, J = 7.7 Hz, 1H), 7.10 (d, J = 7.6 Hz, 1H), 6.83 (dd, J = 5.3, 1.5 Hz, 1H), 6.69- 6.65 (m, 1H), 6.63 (s, 1H), 4.60 (app. p, J = 6.6 Hz, 1H), 4.33 (q, J = 7.0 Hz, 2H), 2.90 (t, J = 7.4 Hz, 2H), 2.60 (t, J = 7.5 Hz, 2H), 1.96 (p, J = 7.5 Hz, 2H), 1.43 (d, J = 6.7 Hz, 6H), 1.34 (t, J = 7.0 Hz, 3H). | m/z 470.6, 492.2 (M + H, M + Na)⁺ (ES⁺) | 469.6 |
| 215 | 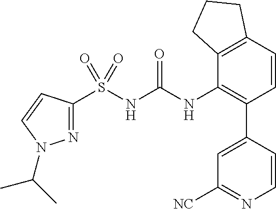 [SMILES: CC(C)n1ccc(S(=O)(=O)NC(=O)Nc2c(-c3ccnc(C#N)c3)ccc3c2CCC3)n1] N-((5-(2-Cyanopyridin-4-yl)-2,3-dihydro- 1H-inden-4-yl)carbamoyl)-1-isopropyl- | ¹H NMR (DMSO-d₆) δ 11.05 (br. s, 1H), 8.67 (d, J = 5.1 Hz, 1H), 8.16 (br. s, 1H), 7.95 (d, J = 1.7 Hz, 1H), 7.89 (d, J = 2.4 Hz, 1H), 7.61 (dd, J = 5.1, 1.8 Hz, 1H), 7.26 (d, J = 7.7 Hz, 1H), 7.19 (d, J = 7.7 Hz, 1H), 6.52 (d, J = 2.3 Hz, 1H), 4.58 (sept, J = 6.6 Hz, 1H), 2.93 (t, J = 7.5 Hz, 2H), 2.68 (t, J = 7.3 Hz, 2H), 1.99 (p, J = 7.5 Hz, 2H), 1.42 (d, J = 6.7 Hz, 6H). | m/z 451.0 (M + H)⁺ (ES⁺) | 450.5 |
| | 1H-pyrazole-3-sulfonamide | | | |
| 216 | 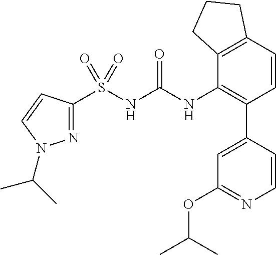 [SMILES: CC(C)Oc1cc(-c2ccc3c(c2NC(=O)NS(=O)(=O)c2ccn(C(C)C)n2)CCC3)ccn1] N-((5-(2-Isopropoxypyridin-4-yl)-2,3- | ¹H NMR (DMSO-d₆) δ 10.84 (br. s, 1H), 8.08 (dd, J = 5.2, 0.7 Hz, 1H), 7.93 (s, 1H), 7.86 (s, 1H), 7.19 (d, J = 7.6 Hz, 1H), 7.09 (d, J = 7.6 Hz, 1H), 6.80 (dd, J = 5.3, 1.5 Hz, 1H), 6.63 (dd, J = 1.5, 0.7 Hz, 1H), 6.61 (s, 1H), 5.27 (app. p, J = 6.2 Hz, 1H), 4.59 (sept, J = 6.7 Hz, 1H), 2.90 (t, J = 7.4 Hz, 2H), 2.60 (t, J = 7.5 Hz, 2H), 1.96 (p, J = 7.5 Hz, 2H), 1.43 (d, J = 6.7 Hz, 6H), 1.31 (d, J = 6.2 Hz, 6H). | m/z 484.4 (M + H)⁺ (ES⁺) | 483.6 |
| | dihydro-1H-inden-4-yl)carbamoyl)-1- | | | |
| | isopropyl-1H-pyrazole-3-sulfonamide | | | |
| 217 | 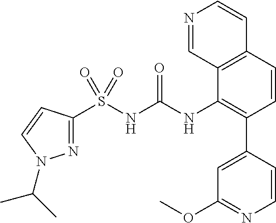 [SMILES: COc1cc(-c2ccc3ccncc3c2NC(=O)NS(=O)(=O)c2ccn(C(C)C)n2)ccn1] 1-Isopropyl-N-((7-(2-methoxypyridin-4- | ¹H NMR (DMSO-d₆) δ 11.32 (br s, 1H), 9.24 (s, 1H), 8.53 (d, J = 5.6 Hz, 1H), 8.40 (br. s, 1H), 8.19 (d, J = 5.2 Hz, 1H), 7.95 (d, J = 8.5 Hz, 1H), 7.86 (d, J = 5.8 Hz, 2H), 7.74 (d, J = 8.5 Hz, 1H), 7.05 (dd, J = 5.2, 1.5 Hz, 1H), 6.91-6.82 (m, 1H), 6.51 (d, J = 6.9 Hz, 1H), 4.60 (hept, J = 6.5 Hz, 1H), 3.91 (s, 3H), 1.45 (d, J = 6.7 Hz, 6H). | m/z 467.3 (M + H)⁺ (ES⁺) | 466.5 |
| | yl)isoquinolin-8-yl)carbamoyl)-1H- | | | |
| | pyrazole-3-sulfonamide | | | |
| 218 | 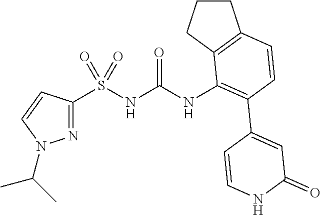 [SMILES: CC(C)n1ccc(S(=O)(=O)NC(=O)Nc2c(-c3cc[nH]c(=O)c3)ccc3c2CCC3)n1] 1-Isopropyl-N-((5-(2-oxo-1,2- | ¹H NMR (DMSO-d6) δ 11.52 (s, 1H), 7.79 (s, 1H), 7.65 (s, 1H), 7.25 (d, J = 6.8 Hz, 1H), 7.11 (d, J = 7.7 Hz, 1H), 7.03 (d, J = 7.6 Hz, 1H), 6.49 (s, 1H), 6.20 (d, J = 1.7 Hz, 1H), 6.15-6.03 (m, 1H), 4.53 (sept, J = 6.7 Hz, 1H), 2.88 (t, J = 7.5 Hz, 2H), 2.64 (t, J = 7.4 Hz, 2H), 1.94 (p, J = 7.5 Hz, 2H), 1.41 (d, J = 6.7 Hz, 6H). One NH not resolved. | m/z 442.1 (M + H)⁺ (ES⁺); 439.9 (M − H)⁻ (ES⁻) | 441.5 |
| | dihydropyridin-4-yl)-2,3-dihydro-1H- | | | |
| | inden-4-yl)carbamoyl)-1H-pyrazole-3- | | | |
| | sulfonamide | | | |
| 219 | 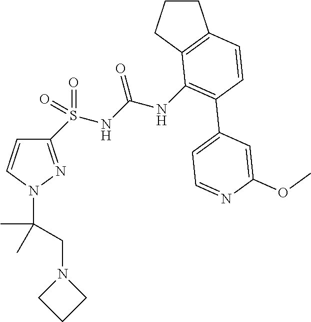 [SMILES: COc1cc(-c2ccc3c(c2NC(=O)NS(=O)(=O)c2ccn(C(C)(C)CN4CCC4)n2)CCC3)ccn1] | 1H NMR (DMSO-d6) δ 10.72 (s, 1H), 8.14 (d, J = 5.3 Hz, 1H), 7.92 (d, J = 2.5 Hz, 1H), 7.86 (s, 1H), 7.20 (d, J = 7.6 Hz, 1H), 7.11 (d, J = 7.6 Hz, 1H), 6.89 (dd, J = 5.3, 1.5 Hz, 1H), 6.73 (d, J = 1.3 Hz, 1H), 6.60 (s, 1H), 3.89 (s, 3H), 3.32 (br s, 2H), 3.03 (br s, 2H), 2.91 (t, J = 7.4 Hz, 2H), 2.80 (br s, 2H), 2.69-2.61 (m, 2H), 1.97 (p, J = 7.6 Hz, 2H), 1.91- 1.78 (m, 2H), 1.50 (s, 6H). | m/z 525.4 (M + H)⁺ (ES⁺); 523.2 (M − H)⁻ (ES⁻) | 524.6 |
| | 1-(1-(Azetidin-1-yl)-2-methylpropan-2- | | | |
| | yl)-N-((5-(2-methoxypyridin-4-yl)-2,3- | | | |
| | dihydro-1H-inden-4-yl)carbamoyl)-1H- | | | |
| | pyrazole-3-sulfonamide | | | |
| 220 | 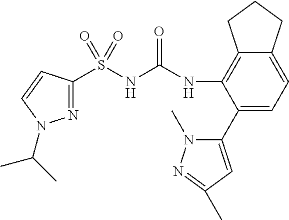 [SMILES: Cc1cc(-c2ccc3c(c2NC(=O)NS(=O)(=O)c2ccn(C(C)C)n2)CCC3)n(C)n1] | 1H NMR (DMSO-d6) δ 10.82 (bs, 1H), 7.96 (d, J = 2.4 Hz, 1H), 7.70 (s, 1H), 7.18 (d, J = 7.6 Hz, 1H), 7.04 (d, J = 7.5 Hz, 1H), 6.65 (s, 1H), 5.93 (s, 1H), 4.62 (sept, J = 6.6 Hz, 1H), 3.36 (s, 3H), 2.91 (t, J = 7.4 Hz, 2H), 2.63 (t, J = 7.4 Hz, 2H), 2.17 (s, 3H), 1.97 (p, J = 7.5 Hz, 2H), 1.49-1.42 (m, 6H). | m/z 443.3 (M + H)⁺ (ES⁺) | 442.5 |
| | N-((5-(1,3-Dimethyl-1H-pyrazol-5-yl)-2,3- | | | |
| | dihydro-1H-inden-4-yl)carbamoyl)-1- | | | |
| | isopropyl-1H-pyrazole-3-sulfonamide | | | |
| 221 | 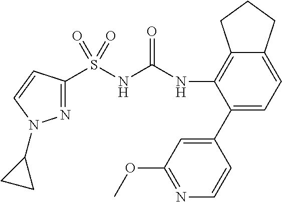 [SMILES: COc1cc(-c2ccc3c(c2NC(=O)NS(=O)(=O)c2ccn(C4CC4)n2)CCC3)ccn1] 1-Cyclopropyl-N-((5-(2-methoxypyridin- | 1H NMR (DMSO-d6) δ 8.04 (d, J = 5.3 Hz, 1H), 7.70 (d, J = 2.4 Hz, 1H), 7.34 (s, 1H), 7.07 (d, J = 7.7 Hz, 1H), 7.03 (d, J = 7.6 Hz, 1H), 6.92 (dd, J = 5.2, 1.5 Hz, 1H), 6.75 (s, 1H), 6.28 (d, J = 2.3 Hz, 1H), 3.86 (s, 3H), 3.71 (tt, J = 7.6, 3.9 Hz, 1H), 2.88 (t, J = 7.5 Hz, 2H), 2.73 (t, J = 7.4 Hz, 2H), 1.95 (p, J = 7.5 Hz, 2H), 1.08-0.91 (m, 4H). | m/z 454.3 (M + H)⁺ (ES⁺); 452.1 (M − H)⁻ (ES⁻) | 453.5 |
| | 4-yl)-2,3-dihydro-1H-inden-4- | | | |
| | yl)carbamoyl)-1H-pyrazole-3- | | | |
| | sulfonamide, sodium salt | | | |
| 222 | 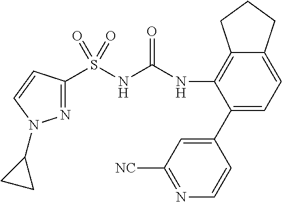 [SMILES: N#Cc1cc(-c2ccc3c(c2NC(=O)NS(=O)(=O)c2ccn(C4CC4)n2)CCC3)ccn1] N-((5-(2-Cyanopyridin-4-yl)-2,3-dihydro- | ¹H NMR (DMSO-d6) δ 8.58 (d, J = 5.2 Hz, 1H), 7.90 (d, J = 1.6 Hz, 1H), 7.64 (d, J = 2.3 Hz, 1H), 7.63 (br s, 1H), 7.62 (dd, J = 5.2, 1.6 Hz, 1H), 7.12 (s, 2H), 6.21 (d, J = 2.3 Hz, 1H), 3.70 (tt, J = 7.5, 3.9 Hz, 1H), 2.90 (t, J = 7.4 Hz, 2H), 2.78 (t, J = 7.4 Hz, 2H), 1.98 (p, J = 7.4 Hz, 2H), 1.04-0.91 (m, 4H). | m/z 449.3 (M + H)⁺ (ES⁺); 447.2 (M − H)⁻ (ES⁻) | 448.5 |
| | 1H-inden-4-yl)carbamoyl)-1-cyclopropyl- | | | |
| | 1H-pyrazole-3-sulfonamide, sodium salt | | | |
| 223 | 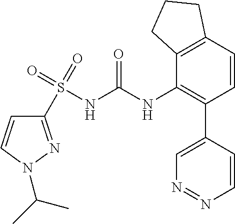 [SMILES: CC(C)n1ccc(S(=O)(=O)NC(=O)Nc2c(-c3ccnnc3)ccc3c2CCC3)n1] | 1H NMR (DMSO-d6) δ 9.19 (dd, J = 2.4, 1.3 Hz, 1H), 9.07 (dd, J = 5.3, 1.3 Hz, 1H), 7.84-7.58 (m, 2H), 7.55 (dd, J = 5.4, 2.4 Hz, 1H), 7.15 (s, 2H), 6.29 (d, J = 2.3 Hz, 1H), 4.51 (sept, J = 6.7 Hz, 1H), 2.90 (t, J = 7.5 Hz, 2H), 2.75 (t, J = 7.5 Hz, 2H), 1.97 (p, J = 7.5 Hz, 2H), 1.41 (d, J = 6.7 Hz, 6H). | m/z 427.3 (M + H)⁺ (ES⁺) | 426.5 |
| | 1-Isopropyl-N-((5-(Pyridazin-4-yl)-2,3- | | | |
| | dihydro-1H-inden-4-yl)carbamoyl)-1H- | | | |
| | pyrazole-3-sulfonamide, sodium salt | | | |
| 224 | 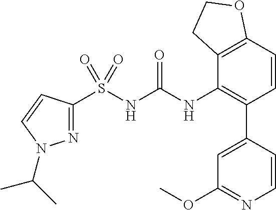 [SMILES: COc1cc(-c2ccc3c(c2NC(=O)NS(=O)(=O)c2ccn(C(C)C)n2)CCO3)ccn1] | ¹H NMR (DMSO-d₆) δ 8.03 (d, J = 5.3 Hz, 1H), 7.71 (d, J = 2.3 Hz, 1H), 7.45 (s, 1H), 7.01 (d, J = 8.2 Hz, 1H), 6.89 (d, J = 5.3 Hz, 1H), 6.73 (s, 1H), 6.60 (d, J = 8.0 Hz, 1H), 6.34 (d, J = 2.3 Hz, 1H), 4.66-4.36 (m, 3H), 3.86 (s, 3H), 3.03 (t, J = 8.8 Hz, 2H), 1.40 (d, J = 6.7 Hz, 6H). | m/z 458.3 (M + H)⁺ (ES⁺) | 457.5 |
| | 1-Isopropyl-N-((5-(2-methoxypyridin-4- | | | |
| | yl)-2,3-dihydrobenzofuran-4- | | | |
| | yl)carbamoyl)-1H-pyrazole-3- | | | |
| | sulfonamide, sodium salt | | | |
| 225 | 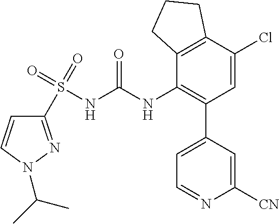 [SMILES: CC(C)n1ccc(S(=O)(=O)NC(=O)Nc2c(-c3ccnc(C#N)c3)cc(Cl)c3c2CCC3)n1] | 1H NMR (DMSO-d6) δ 8.66 (d, J = 5.0 Hz, 1H), 7.97 (d, J = 1.6 Hz, 1H), 7.76 (s, 1H), 7.65 (dd, J = 5.0, 1.6 Hz, 1H), 7.29 (s, 1H), 7.09 (s, 1H), 6.37 (s, 1H), 4.54 (hept, J = 6.7 Hz, 1H), 2.96 (t, J = 7.5 Hz, 2H), 2.83 (t, J = 7.5 Hz, 2H), 2.04 (p, J = 7.5 Hz, 2H), 1.42 (d, J = 6.7 Hz, 6H). One NH not observed. | m/z 485.2, 487.2 (M + H)⁺ (ES⁺); 482.7 (M − H)⁻ (ES⁻) | 485 |
| | N-((7-Chloro-5-(2-cyanopyridin-4-yl)-2,3- | | | |
| | dihydro-1H-inden-4-yl)carbamoyl)-1- | | | |
| | isopropyl-1H-pyrazole-3-sulfonamide, | | | |
| | sodium salt | | | |
| 226 | 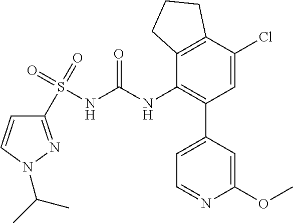 [SMILES: COc1cc(-c2cc(Cl)c3c(c2NC(=O)NS(=O)(=O)c2ccn(C(C)C)n2)CCC3)ccn1] | 1H NMR (DMSO-d6) δ 8.04 (d, J = 5.3 Hz, 1H), 7.72 (d, J = 2.3 Hz, 1H), 7.44 (s, 1H), 7.10 (s, 1H), 6.91 (d, J = 5.3, Hz, 1H), 6.78 (s, 1H), 6.31 (d, J = 2.3 Hz, 1H), 4.50 (hept, J = 6.7 Hz, 1H), 3.86 (s, 3H), 2.91 (t, J = 7.6 Hz, 2H), 2.79 (t, J = 7.6 Hz, 2H), 1.98 (p, J = 7.6 Hz, 2H), 1.40 (d, J = 6.7 Hz, 6H). | m/z 489.9, 492.5 (M + H)⁺ (ES⁺); 488.2, 490.2 (M − H)⁻ (ES⁻). | 489.98 |
| | N-((7-Chloro-5-(2-methoxypyridin-4-yl) | | | |
| | 2,3-dihydro-1H-inden-4-yl)carbamoyl)-1- | | | |
| | isopropyl-1H-pyrazole-3-sulfonamide, | | | |
| | sodium salt | | | |
| 227 | 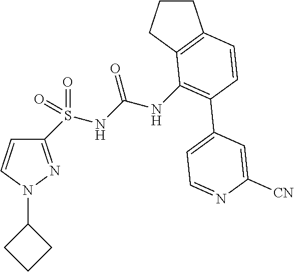 [SMILES: N#Cc1cc(-c2ccc3c(c2NC(=O)NS(=O)(=O)c2ccn(C4CCC4)n2)CCC3)ccn1] | 1H NMR (DMSO-d6) δ 8.62 (d, J = 5.1 Hz, 1H), 7.96-7.83 (m, 2H), 7.80 (s, 1H), 7.63 (dd, J = 5.1, 1.8 Hz, 1H), 7.21-7.15 (m, 2H), 6.37 (s, 1H), 4.86 (p, J = 8.5 Hz, 1H), 2.92 (t, J = 7.5 Hz, 2H), 2.75 (t, J = 7.5 Hz, 2H), 2.47-2.42 (m, 2H), 2.41-2.37 (m, 2H), 2.00 (p, J = 7.5 Hz, 2H), 1.83-1.74 (m, 2H). NH proton not observed. | m/z 463.3 (M + H)⁺ (ES⁺) | 462.5 |
| | N-((5-(2-Cyanopyridin-4-yl)-2,3-dihydro- | | | |
| | 1H-inden-4-yl)carbamoyl)-1-cyclobutyl- | | | |
| | 1H-pyrazole-3-sulfonamide | | | |
| 228 | 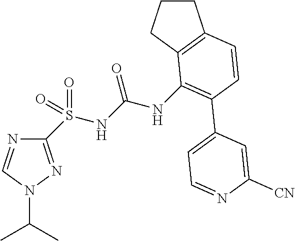 [SMILES: CC(C)n1cnc(S(=O)(=O)NC(=O)Nc2c(-c3ccnc(C#N)c3)ccc3c2CCC3)n1] | 1H NMR (DMSO-d6) δ 8.58 (d, J = 5.1 Hz, 1H), 8.07 (s, 1H), 7.93 (d, J = 1.7 Hz, 1H), 7.75-7.56 (m, 2H), 7.16-7.09 (m, 2H) 4.80 (sept, J = 6.8 Hz, 1H), 2.90 (t, J = 7.5 Hz, 2H), 2.76 (t, J = 7.4 Hz, 2H), 1.98 (p, J = 7.5 Hz, 2H), 1.50 (d, J = 6.7 Hz, 6H). | m/z 452.3 (M + H)⁺ (ES⁺) | 451.5 |
| | N-((5-(2-Cyanopyridin-4-yl)-2,3-dihydro- | | | |
| | 1H-inden-4-yl)carbamoyl)-1-isopropyl- | | | |
| | 1H-1,2,4-triazole-3-sulfonamide, sodium | | | |
| | salt | | | |
| 229 | 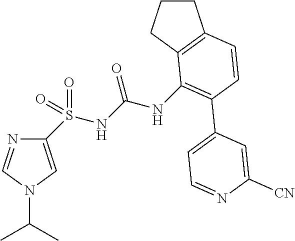 [SMILES: CC(C)n1cnc(S(=O)(=O)NC(=O)Nc2c(-c3ccnc(C#N)c3)ccc3c2CCC3)c1] N-((5-(2-Cyanopyridin-4-yl)-2,3-dihydro- | ¹H NMR (DMSO-d6) δ 8.65 (dd, J = 5.1, 0.8 Hz, 1H), 8.13 (s, 1H), 7.94 (dd, J = 1.8, 0.8 Hz, 1H), 7.87 (s, 1H), 7.77 (s, 1H), 7.60 (dd, J = 5.1, 1.8 Hz, 1H), 7.23 (d, J = 7.7 Hz, 1H), 7.19 (d, J = 7.7 Hz, 1H), 4.46 (sept, J = 6.8 Hz, 1H), 2.93 (t, J = 7.5 Hz, 2H), 2.71 (t, J = 7.5 Hz, 2H), 2.01 (p, J = 7.5 Hz, 2H), 1.42 (d, J = 6.7 Hz, 6H). One NH not observed. | m/z 451.2 (M + H)⁺ (ES⁺) | 450.5 |
| | 1H-inden-4-yl)carbamoyl)-1-isopropyl- | | | |
| | 1H-imidazole-4-sulfonamide | | | |
| 230 | 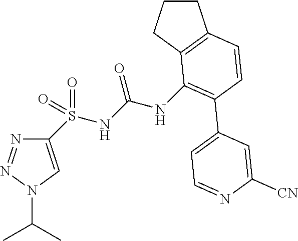 [SMILES: CC(C)n1cc(S(=O)(=O)NC(=O)Nc2c(-c3ccnc(C#N)c3)ccc3c2CCC3)nn1] | ¹H NMR (DMSO-d6) δ 8.58 (d, J = 5.1 Hz, 1H), 8.07 (s, 1H), 7.93 (d, J = 1.7 Hz, 1H), 7.75-7.56 (m, 2H), 7.16-7.09 (m, 2H), 4.80 (sept, J = 6.8 Hz, 1H), 2.90 (t, J = 7.5 Hz, 2H), 2.76 (t, J = 7.4 Hz, 2H), 1.98 (p, J = 7.5 Hz, 2H), 1.50 (d, J = 6.7 Hz, 6H). | m/z 452.3 (M + H)⁺ (ES⁺) | 451.5 |
| | N-((5-(2-Cyanopyridin-4-yl)-2,3-dihydro- | | | |
| | 1H-inden-4-yl)carbamoyl)-1-isopropyl- | | | |
| | 1H-1,2,3-triazole-4-sulfonamide, sodium | | | |
| | salt | | | |
| 231 | 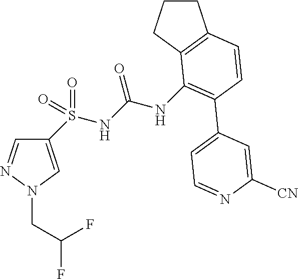 [SMILES: N#Cc1cc(-c2ccc3c(c2NC(=O)NS(=O)(=O)c2cnn(CC(F)F)c2)CCC3)ccn1] | ¹H NMR (DMSO-d6) δ 8.54 (dd, J = 5.1, 0.8 Hz, 1H), 7.93-7.87 (m, 1H), 7.82 (s, 1H), 7.60 (dd, J = 5.1, 1.7 Hz, 1H), 7.51 (s, 1H), 7.48 (s, 1H), 7.15- 7.08 (m, 2H), 6.35 (tt, J = 55.0, 3.9 Hz, 1H), 4.59 (td, J = 15.0, 3.8 Hz, 2H), 2.91 (t, J = 7.5 Hz, 2H), 2.78 (t, J = 7.5 Hz, 2H), 1.98 (p, J = 7.5 Hz, 2H). | m/z 473.3 (M + H)⁺ (ES⁺) | 472.5 |
| | N-((5-(2-Cyanopyridin-4-yl)-2,3-dihydro- | | | |
| | 1H-inden-4-yl)carbamoyl)-1-(2,2- | | | |
| | difluoroethyl)-1H-pyrazole-4- | | | |
| | sulfonamide, sodium salt | | | |
| 232 | 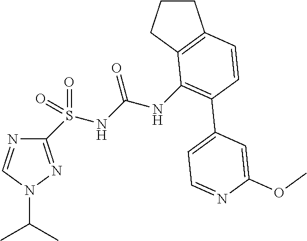 [SMILES: COc1cc(-c2ccc3c(c2NC(=O)NS(=O)(=O)c2ncn(C(C)C)n2)CCC3)ccn1] | 1H NMR (DMSO-d6) δ 8.55 (s, 1H), 8.06 (d, J = 5.3 Hz, 1H), 7.50 (s, 1H), 7.09 (d, J = 7.7 Hz, 1H), 7.04 (d, J = 7.6 Hz, 1H), 6.96 (d, J = 3.9 Hz, 1H), 6.76 (d, J = 1.4 Hz, 1H), 4.62 (sept, J = 6.7 Hz, 1H), 3.86 (s, 3H), 2.88 (t, J = 7.4 Hz, 2H), 2.70 (t, J = 7.5 Hz, 2H), 1.94 (p, J = 7.5 Hz, 2H), 1.45 (d, J = 6.7 Hz, 6H). | m/z 457.3 (M + H)⁺ (ES⁺) | 456.5 |
| | 1-Isopropyl-N-((5-(2-methoxypyridin-4- | | | |
| | yl)-2,3-dihydro-1H-inden-4- | | | |
| | yl)carbamoyl)-1H-1,2,4-triazole-3- | | | |
| | sulfonamide, sodium salt | | | |
| 233 | 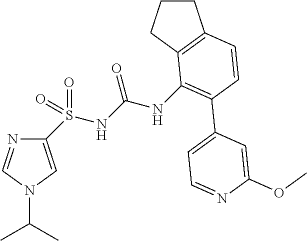 [SMILES: COc1cc(-c2ccc3c(c2NC(=O)NS(=O)(=O)c2cn(C(C)C)cn2)CCC3)ccn1] | 1H NMR (DMSO-d6) δ 8.11 (d, J = 5.3 Hz, 1H), 7.92-7.80 (m, 3H), 7.18 (d, J = 7.7 Hz, 1H), 7.10 (d, J = 7.6 Hz, 1H), 6.83 (d, J = 5.3 Hz, 1H), 6.70 (s, 1H), 4.48 (sept, J = 6.5 Hz, 1H), 3.88 (s, 3H), 2.90 (t, J = 7.4 Hz, 2H), 2.61 (t, J = 7.4 Hz, 2H), 1.96 (p, J = 7.5 Hz, 2H), 1.42 (d, J = 6.6 Hz, 6H). One NH not observed. | m/z 456.3 (M + H)⁺ (ES⁺) | 455.5 |
| | 1-Isopropyl-N-((5-(2-methoxypyridin-4- | | | |
| | yl)-2,3-dihydro-1H-inden-4- | | | |
| | yl)carbamoyl)-1H-imidazole-4- | | | |
| | sulfonamide | | | |
| 234 | 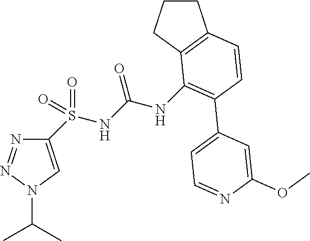 [SMILES: COc1cc(-c2ccc3c(c2NC(=O)NS(=O)(=O)c2cn(C(C)C)nn2)CCC3)ccn1] | 1H NMR (DMSO-d6) δ 8.20 (s, 1H), 8.03 (d, J = 5.3 Hz, 1H), 7.41 (s, 1H), 7.08 (d, J = 7.7 Hz, 1H), 7.04 (d, J = 7.6 Hz, 1H), 6.91 (d, J = 6.7 Hz, 1H), 6.75 (s, 1H), 4.82 (sept, J = 6.7 Hz, 1H), 3.85 (s, 3H), 2.88 (t, J = 7.4 Hz, 2H), 2.68 (t, J = 7.4 Hz, 2H), 1.94 (p, J = 7.5 Hz, 2H), 1.50 (d, J = 6.7 Hz, 6H). | m/z 457.3 (M + H)⁺ (ES⁺) | 456.5 |
| | 1-Isopropyl-N-((5-(2-methoxypyridin-4- | | | |
| | yl)-2,3-dihydro-1H-inden-4- | | | |
| | yl)carbamoyl)-1H-1,2,3-triazole-4- | | | |
| | sulfonamide, sodium salt | | | |
| 235 | 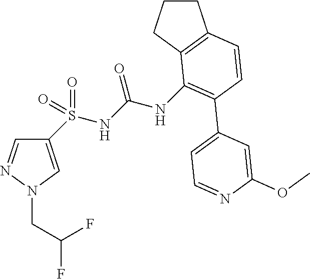 [SMILES: COc1cc(-c2ccc3c(c2NC(=O)NS(=O)(=O)c2cnn(CC(F)F)c2)CCC3)ccn1] | 1H NMR (DMSO-d6) δ 8.02 (d, J = 5.3 Hz, 1H), 7.90 (s, 1H), 7.59 (s, 1H), 7.21 (s, 1H), 7.08 (d, J = 7.6 Hz, 1H), 7.03 (d, J = 7.6 Hz, 1H), 6.90 (dd, J = 5.3, 1.5 Hz, 1H), 6.75 (d, J = 1.4 Hz, 1H), 6.35 (tt, J = 54.9, 3.8 Hz, 1H), 4.61 (td, J = 15.1, 3.8 Hz, 2H), 3.85 (s, 3H), 2.88 (t, J = 7.4 Hz, 2H), 2.74 (t, J = 7.3 Hz, 2H), 1.95 (p, J = 7.5 Hz, 2H). | m/z 478.3 (M + H)⁺ (ES⁺) | 477.5 |
| | 1-(2,2-Difluoroethyl)-N-((5-(2- | | | |
| | methoxypyridin-4-yl)-2,3-dihydro-1H- | | | |
| | inden-4-yl)carbamoyl)-1H-pyrazole-4- | | | |
| | sulfonamide, sodium salt | | | |
| 236 | 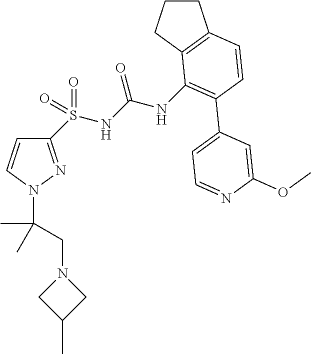 [SMILES: COc1cc(-c2ccc3c(c2NC(=O)NS(=O)(=O)c2ccn(C(C)(C)CN4CC(C)C4)n2)CCC3)ccn1] | 1H NMR (DMSO-d6) δ 8.13 (d, J = 5.3 Hz, 1H), 7.87 (s, 1H), 7.76 (s, 1H), 7.17 (d, J = 7.7 Hz, 1H), 7.09 (d, J = 7.6 Hz, 1H), 6.90 (dd, J = 5.3, 1.4 Hz, 1H), 6.74 (s, 1H), 6.53 (s, 1H), 3.89 (s, 3H), 3.20 (s, 2H), 2.90 (t, J = 7.4 Hz, 2H), 2.77 (s, 2H), 2.73- 2.55 (m, 4H), 2.35-2.24 (m, 1H), 1.97 (p, J = 7.5 Hz, 2H), 1.48 (s, 6H), 0.93 (d, J = 6.7 Hz, 3H). One NH not observed. | m/z 539.3 (M + H)⁺ (ES⁺); 537.1 (M − H)⁻ (ES⁻) | 538.7 |
| | N-((5-(2-Methoxypyridin-4-yl)-2,3- | | | |
| | dihydro-1H-inden-4-yl)carbamoyl)-1-(2- | | | |
| | methyl-1-(3-methylazetidin-1-yl)propan- | | | |
| | 2-yl)-1H-pyrazole-3-sulfonamide | | | |
| 237 | 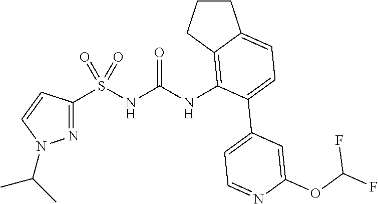 [SMILES: CC(C)n1ccc(S(=O)(=O)NC(=O)Nc2c(-c3ccnc(OC(F)F)c3)ccc3c2CCC3)n1] N-((5-(2-(Difluoromethoxy)pyridin-4-yl)- | 1H NMR (DMSO-d6) δ 8.11 (d, J = 5.3 Hz, 1H), 7.73 (t, J = 73.1 Hz, 1H), 7.69-7.66 (m, 1H), 7.50 (s, 1H), 7.19 (d, J = 5.2 Hz, 1H), 7.13-7.05 (m, 2H), 7.00 (s, 1H), 6.28 (t, J = 1.7 Hz, 1H), 4.49 (sept, J = 6.8 Hz, 1H), 2.89 (t, J = 7.5 Hz, 2H), 2.74 (t, J = 7.4 Hz, 2H), 1.96 (p, J = 7.5 Hz, 2H), 1.40 (d, J = 6.7 Hz, 6H). | m/z 492.3 (M + H)⁺ (ES⁺); 490.0 (M − H)⁻ (ES⁻), | 491.5 |
| | 2,3-dihydro-1H-inden-4-yl)carbamoyl)-1- | | | |
| | isopropyl-1H-pyrazole-3-sulfonamide, | | | |
| | sodium salt | | | |
| 238 | 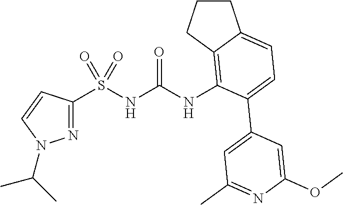 [SMILES: COc1cc(-c2ccc3c(c2NC(=O)NS(=O)(=O)c2ccn(C(C)C)n2)CCC3)cc(C)n1] 1-Isopropyl-N-((5-(2-methoxy-6- | 1H NMR (DMSO-d6) δ 7.68 (d, J = 2.3 Hz, 1H), 7.33 (s, 1H), 7.06 (d, J = 7.7 Hz, 1H), 7.01 (d, J = 7.6 Hz, 1H), 6.78 (s, 1H), 6.56 (s, 1H), 6.31 (d, J = 2.2 Hz, 1H), 4.48 (sept, J = 6.7 Hz, 1H), 3.84 (s, 3H), 2.87 (t, J = 7.5 Hz, 2H), 2.70 (t, J = 7.5 Hz, 2H), 2.35 (s, 3H), 1.93 (p, J = 7.5 Hz, 2H), 1.40 (d, J = 6.7 Hz, 6H). | m/z 470.3 (M + H)⁺ (ES⁺); 468.2 (M − H)⁻ (ES⁻) | 469.6 |
| | methylpyridin-4-yl)-2,3-dihydro-1H- | | | |
| | inden-4-yl)carbamoyl)-1H-pyrazole-3- | | | |
| | sulfonamide, sodium salt | | | |
| 239 | 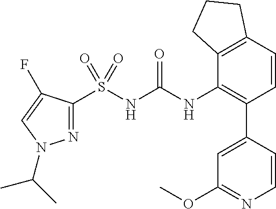 [SMILES: COc1cc(-c2ccc3c(c2NC(=O)NS(=O)(=O)c2nn(C(C)C)cc2F)CCC3)ccn1] 4-Fluoro-1-isopropyl-N-((5-(2- | ¹H NMR (DMSO-d₆) δ 11.14 (br. s, 1H), 8.12 (d, J = 5.2 Hz, 1H), 8.08 (s, 1H), 7.79 (br. s,1H), 7.19 (d, J = 7.6 Hz, 1H), 7.10 (d, J = 7.7 Hz, 1H), 6.91- 6.86 (m, 1H), 6.73 (d, J = 1.4 Hz, 1H), 4.53-4.41 (m, 1H), 3.88 (s, 3H), 2.91 (t, J = 7.4 Hz, 2H), 2.69- 2.63 (m, 2H), 1.97 (p, J = 7.5 Hz, 2H), 1.40 (d, J = 6.7 Hz, 6H). | m/z 474.3 (M + H)⁺ (ES⁺) | 473.5 |
| | methoxypyridin-4-yl)-2,3-dihydro-1H- | | | |
| | inden-4-yl)carbamoyl)-1H-pyrazole-3- | | | |
| | sulfonamide | | | |
| 240 | 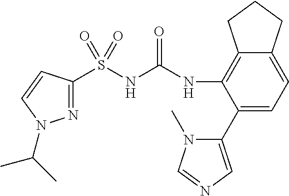 [SMILES: CC(C)n1ccc(S(=O)(=O)NC(=O)Nc2c(-c3cncn3C)ccc3c2CCC3)n1] 1-Isopropyl-N-((5-(1-methyl-1H-imidazol- | 1H NMR (DMSO-d6) δ 7.89 (d, J = 2.4 Hz, 1H), 7.70 (s, 1H), 7.57 (s, 1H), 7.14 (d, J = 7.6 Hz, 1H), 7.03 (d, J = 7.6 Hz, 1H), 6.77 (s, 1H), 6.56 (s, 1H), 4.59 (sept, J = 6.6 Hz, 1H), 3.34 (s, 3H), 2.89 (t, J = 7.5 Hz, 2H), 2.64 (t, J = 7.4 Hz, 2H), 1.95 (p, J = 7.4 Hz, 2H), 1.44 (d, J = 6.7 Hz, 6H). Free acid not observed. | m/z 429.3 (M + H)⁺ (ES⁺) at 0.83 min, 96% purity (254 nm). | 428.5 |
| | 5-yl)-2,3-dihydro-1H-inden-4- | | | |
| | yl)carbamoyl)-1H-pyrazole-3-sulfonamide | | | |
| 241 | 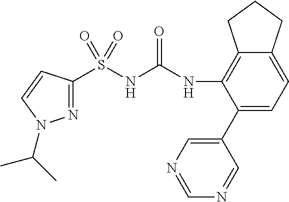 [SMILES: CC(C)n1ccc(S(=O)(=O)NC(=O)Nc2c(-c3cncnc3)ccc3c2CCC3)n1] | 1H NMR (DMSO-d6) δ 9.08 (s, 1H), 8.73 (s, 2H), 7.68 (d, J = 2.3 Hz, 1H), 7.60 (s, 1H), 7.15-7.09 (m, 2H), 6.28 (d, J = 2.3 Hz, 1H), 4.49 (sept, J = 6.7 Hz, 1H), 2.91 (t, J = 7.5 Hz, 2H), 2.75 (t, J = 7.4 Hz, 2H), 1.97 (p, J = 7.5 Hz, 2H), 1.41 (d, J = 6.7 Hz, 6H). One NH not observed. | m/z 427.2 (M + H)⁺ (ES⁺) | 426.5 |
| | 1-Isopropyl-N-((5-(pyrimidin-5-yl)-2,3- | | | |
| | dihydro-1H-inden-4-yl)carbamoyl)-1H- | | | |
| | pyrazole-3-sulfonamide | | | |
| 242 | 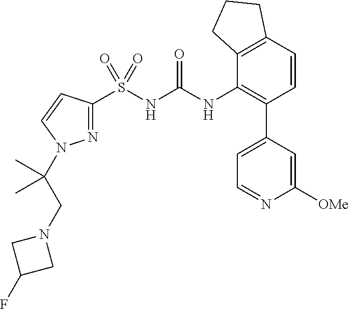 [SMILES: COc1cc(-c2ccc3c(c2NC(=O)NS(=O)(=O)c2ccn(C(C)(C)CN4CC(F)C4)n2)CCC3)ccn1] | 1H NMR (DMSO-d6) δ 8.07 (d, J = 5.3 Hz, 1H), 7.65 (d, J = 2.4 Hz, 1H), 7.09-7.00 (m, 2H), 6.95 (d, J = 5.3 Hz, 1H), 6.78 (s, 1H), 6.29 (d, J = 2.3 Hz, 1H), 5.07-4.86 (m, 1H), 3.87 (s, 3H), 3.29- 3.21 (m, 2H), 3.01-2.90 (m, 2H), 2.87 (t, J = 7.4 Hz, 2H), 2.76 (s, 2H), 2.73 (t, J = 7.7 Hz, 2H), 1.94 (p, J = 7.6 Hz, 2H), 1.46 (s, 6H). One exchangeable proton not observed. | m/z 543.3 (M + H)+ (ES+); 541.3 (M − H)− (ES−) | 542.63 |
| | 1-(1-(3-Fluoroazetidin-1-yl)-2- | | | |
| | methylpropan-2-yl)-N-((5-(2- | | | |
| | methoxypyridin-4-yl)-2,3-dihydro-1H- | | | |
| | inden-4-yl)carbamoyl)-1H-pyrazole-3- | | | |
| | sulfonamide, sodium salt | | | |
| 243 | 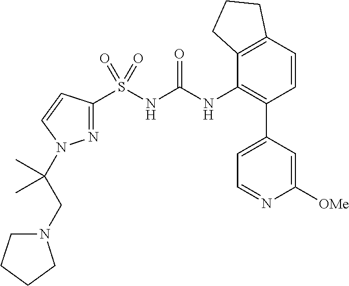 [SMILES: COc1cc(-c2ccc3c(c2NC(=O)NS(=O)(=O)c2ccn(C(C)(C)CN4CCCC4)n2)CCC3)ccn1] | 1H NMR (DMSO-d6) δ 8.06 (d, J = 5.3 Hz, 1H), 7.67 (d, J = 2.3 Hz, 1H), 7.33 (s, 1H), 7.06 (d, J = 7.7 Hz, 1H), 7.02 (d, J = 7.6 Hz, 1H), 6.93 (d, J = 5.2 Hz, 1H), 6.76 (s, 1H), 6.30 (d, J = 2.3 Hz, 1H), 3.86 (s, 3H), 2.86 (t, J = 7.5 Hz, 2H), 2.74 (s, 2H), 2.70 (t, J = 7.5 Hz, 2H), 2.27-2.20 (m, 4H), 1.92 (p, J = 7.4 Hz, 2H), 1.53- 1.50 (m, 4H), 1.49 (s, 6H). | m/z 539.5 (M + H)+ (ES+); 537.8 (M − H)− (ES−) | 538.66 |
| | N-((5-(2-Methoxypyridin-4-yl)-2,3- | | | |
| | dihydro-1H-inden-4-yl)carbamoyl)-1-(2- | | | |
| | methyl-1-(pyrrolidin-1-yl)propan-2-yl)- | | | |
| | 1H-pyrazole-3-sulfonamide, sodium salt | | | |
| 244 | 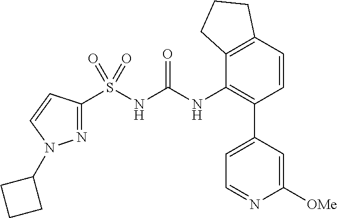 [SMILES: COc1cc(-c2ccc3c(c2NC(=O)NS(=O)(=O)c2ccn(C4CCC4)n2)CCC3)ccn1] 1-Cyclobutyl-N-((5-(2-methoxypyridin-4- | 1H NMR (DMSO-d6) δ 8.03 (d, J = 5.3 Hz, 1H), 7.76-7.73 (m, 1H), 7.35 (s, 1H), 7.07 (d, J = 7.7 Hz, 1H), 7.03 (d, J = 7.6 Hz, 1H), 6.94-6.90 (m, 1H), 6.76 (s, 1H), 6.33- 6.29 (m, 1H), 4.82 (p, J = 8.4 Hz, 1H), 3.86 (s, 3H), 2.88 (t, J = 7.4 Hz, 2H), 2.72 (t, J = 7.5 Hz, 2H), 2.49-2.40 (m, 2H), 2.40- 2.29 (m, 2H), 1.94 (p, J = 7.5 Hz, 2H), 1.83-1.70 (m, 2H). | m/z 468.0 (M + H)+ (ES+); 466.4 (M − H)− (ES−) | 467.54 |
| | yl)-2,3-dihydro-1H-inden-4- | | | |
| | yl)carbamoyl)-1H-pyrazole-3- | | | |
| | sulfonamide, sodium salt | | | |
| 245 | 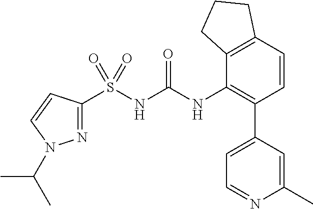 [SMILES: Cc1cc(-c2ccc3c(c2NC(=O)NS(=O)(=O)c2ccn(C(C)C)n2)CCC3)ccn1] 1-Isopropyl-N-((5-(2-methylpyridin-4-yl)- | 1H NMR (DMSO-d6) δ 8.29 (d, J = 5.1 Hz, 1H), 7.73 (d, J = 2.3 Hz, 1H), 7.38 (s, 1H), 7.18 (s, 1H), 7.13-7.06 (m, 2H), 7.03 (d, J = 7.6 Hz, 1H), 6.33 (d, J = 2.3 Hz, 1H), 4.51 (sept, J = 6.6 Hz, 1H), 2.88 (t, J = 7.4 Hz, 2H), 2.73 (t, J = 7.5 Hz, 2H), 2.43 (s, 3H), 1.94 (p, J = 7.5 Hz, 2H), 1.40 (d, J = 6.7 Hz, 6H). | m/z 440.4 (M + H)+ (ES+) | 439.53 |
| | 2,3-dihydro-1H-inden-4-yl)carbamoyl)- | | | |
| | 1H-pyrazole-3-sulfonamide, sodium salt | | | |
| 246 | 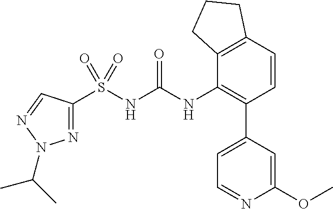 [SMILES: COc1cc(-c2ccc3c(c2NC(=O)NS(=O)(=O)c2cnn(C(C)C)n2)CCC3)ccn1] 2-Isopropyl-N-((5-(2-methoxypyridin-4- | 1H NMR (DMSO-d6) δ 8.06 (d, J = 5.3 Hz, 1H), 7.64-7.60 (m, 1H), 7.30 (s, 1H), 7.08 (d, J = 7.7 Hz, 1H), 7.03 (d, J = 7.6 Hz, 1H), 6.93 (dd, J = 5.3, 1.5 Hz, 1H), 6.77 (s, 1H), 4.79 (sept, J = 6.7 Hz, 1H), 3.86 (s, 3H), 2.88 (t, J = 7.4 Hz, 2H), 2.71 (t, J = 7.5 Hz, 2H), 1.95 (p, J = 7.5 Hz, 2H), 1.48 (d, J = 6.7 Hz, 6H). | m/z 457.3 (M + H)+ (ES+); 455.2 (M − H)− (ES−) | 456.52 |
| | yl)-2,3-dihydro-1H-inden-4- | | | |
| | yl)carbamoyl)-2H-1,2,3-triazole-4- | | | |
| | sulfonamide, sodium salt | | | |
| 247 | 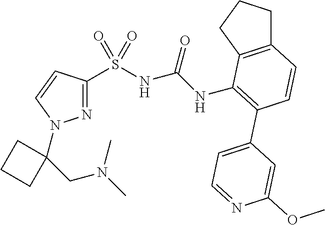 [SMILES: COc1cc(-c2ccc3c(c2NC(=O)NS(=O)(=O)c2ccn(C4(CN(C)C)CCC4)n2)CCC3)ccn1] 1-(1-((Dimethylamino)methyl)cyclobutyl)- | 1H NMR (DMSO-d6) δ 8.07 (d, J = 5.3 Hz, 1H), 7.64 (d, J = 2.3 Hz, 1H), 7.32 (s, 1H), 7.06 (d, J = 7.7 Hz, 1H), 7.03 (d, J = 7.7 Hz, 1H), 6.97-6.92 (m, 1H), 6.77 (d, J = 1.5 Hz, 1H), 6.33 (d, J = 2.2, Hz, 1H), 3.87 (s, 3H), 2.07 (t, J = 7.5 Hz, 2H), 2.72 (t, J = 7.4 Hz, 2H), 2.69 (s, 2H), 2.48-2.40 (m, 2H), 2.31-2.23 (m, 2H), 2.00- 1.78 (m, 4H), 1.91 (s, 6H). | m/z 525.3 (M + H)+ (ES+); 523.2 (M − H)− (ES−) | 524.64 |
| | N-((5-(2-methoxypyridin-4-yl)-2,3- | | | |
| | dihydro-1H-inden-4-yl)carbamoyl)-1H- | | | |
| | pyrazole-3-sulfonamide, sodium salt | | | |
| 248 | 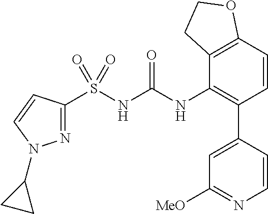 [SMILES: COc1cc(-c2ccc3c(c2NC(=O)NS(=O)(=O)c2ccn(C4CC4)n2)CCO3)ccn1] 1-Cyclopropyl-N-((5-(2-methoxypyridin-4- | 1H NMR (DMSO-d6) δ 8.06 (d, J = 5.3 Hz, 1H), 7.68 (d, J = 2.3 Hz, 1H), 7.38 (s, 1H), 7.01 (d, J = 8.2 Hz, 1H), 6.91 (dd, J = 5.3, 1.5 Hz, 1H), 6.73 (d, J = 1.4 Hz, 1H), 6.61 (d, J = 8.2 Hz, 1H), 6.30 (d, J = 2.3 Hz, 1H), 4.51 (t, J = 8.8 Hz, 2H), 3.86 (s, 3H), 3.70 (tt, J = 7.4, 3.9 Hz, 1H), 3.06 (t, J = 8.8 Hz, 2H), 1.06-0.99 (m, 2H), 0.98-0.92 (m, 2H). | m/z 456.3 (M + H)+ (ES+) | 455.49 |
| | 3-sulfonamide, sodium salt | | | |
| 249 | 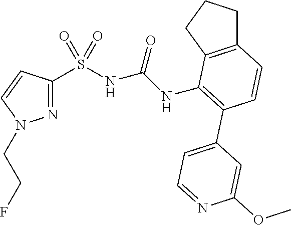 [SMILES: COc1cc(-c2ccc3c(c2NC(=O)NS(=O)(=O)c2ccn(CCF)n2)CCC3)ccn1] | 1H NMR (DMSO-d6) δ 8.05-8.00 (m, 1H), 7.70 (d, J = 2.3 Hz, 1H), 7.34 (s, 1H), 7.07 (d, J = 7.7 Hz, 1H), 7.03 (d, J = 7.6 Hz, 1H), 6.90 (dd, J = 5.3, 1.5 Hz, 1H), 6.75 (d, J = 1.4 Hz, 1H), 6.32 (d, J = 2.3 Hz, 1H), 4.83-4.36 (m, 4H), 3.86 (s, 3H), 2.88 (t, J = 7.4 Hz, 2H), 2.73 (t, J = 7.5 Hz, 2H), 2.00-1.90 (m, 2H). | m/z 460.3 (M + H)+ (ES+) | 459.49 |
| | 1-(2-Fluoroethyl)-N-((5-(2- | | | |
| | methoxypyridin-4-yl)-2,3-dihydro-1H- | | | |
| | inden-4-yl)carbamoyl)-1H-pyrazole-3- | | | |
| | sulfonamide, sodium salt | | | |
| 250 | 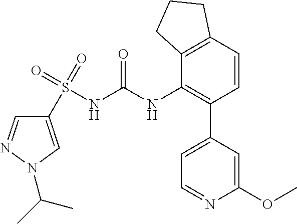 [SMILES: COc1cc(-c2ccc3c(c2NC(=O)NS(=O)(=O)c2cnn(C(C)C)c2)CCC3)ccn1] 1-Isopropyl-N-((5-(2-methoxypyridin-4- | 1H NMR (DMSO-d6) δ 8.01 (dd, J = 5.3, 0.7 Hz, 1H), 7.84 (d, J = 0.6 Hz, 1H), 7.49 (d, J = 0.6 Hz, 1H), 7.18 (s, 1H), 7.07 (d, J = 7.7 Hz, 1H), 7.03 (d, J = 7.6 Hz, 1H), 6.90 (dd, J = 5.3, 1.5 Hz, 1H), 6.79- 6.70 (m, 1H), 4.46 (hept, J = 6.7 Hz, 1H), 3.85 (s, 3H), 2.88 (t, J = 7.4 Hz, 2H), 2.72 (t, J = 7.5 Hz, 2H), 1.94 (p, J = 7.5 Hz, 2H), 1.40 (d, J = 6.7 Hz, 6H). | m/z 456.3 (M + H)+ (ES+); 454.2 (M − H)− (ES−) | 455.53 |
| | yl)-2,3-dihydro-1H-inden-4- | | | |
| | yl)carbamoyl)-1H-pyrazole-4- | | | |
| | sulfonamide, sodium salt | | | |
| 251 | 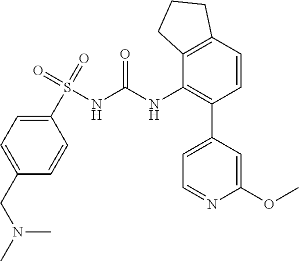 [SMILES: COc1cc(-c2ccc3c(c2NC(=O)NS(=O)(=O)c2ccc(CN(C)C)cc2)CCC3)ccn1] | 1H NMR (DMSO-d6) δ 8.04-7.93 (m, 1H), 7.65- 7.49 (m, 2H), 7.26-7.21 (m, 2H), 7.19 (s, 1H), 7.04 (d, J = 7.6 Hz, 1H), 7.00 (d, J = 7.6 Hz, 1H), 6.84 (dd, J = 5.3, 1.5 Hz, 1H), 6.75-6.68 (m, 1H), 3.86 (s, 3H), 3.38 (s, 2H), 2.85 (t, J = 7.4 Hz, 2H), 2.68 (t, J = 7.4 Hz, 2H), 2.13 (s, 6H), 1.91 (p, J = 7.4 Hz, 2H). | m/z 481.3 (M + H)+ (ES+); 479.2 (M − H)− (ES−) | 480.58 |
| | 4-((Dimethylamino)methyl)-N-((5-(2- | | | |
| | methoxypyridin-4-yl)-2,3-dihydro-1H- | | | |
| | inden-4- | | | |
| | yl)carbamoyl)benzenesulfonamide, | | | |
| | sodium salt | | | |
| 252 | 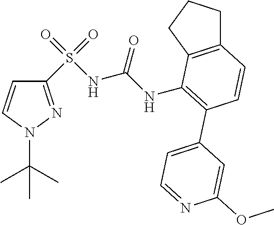 [SMILES: COc1cc(-c2ccc3c(c2NC(=O)NS(=O)(=O)c2ccn(C(C)(C)C)n2)CCC3)ccn1] | 1H NMR (DMSO-d6) δ 8.05 (d, J = 5.4 Hz, 1H), 7.72 (d, J = 2.3 Hz, 1H), 7.33 (br s, 1H), 7.07 (d, J = 7.7 Hz, 1H), 7.03 (d, J = 7.6 Hz, 1H), 6.94 (dd, J = 5.3, 1.5 Hz, 1H), 6.77 (d, J = 1.3 Hz, 1H), 6.30 (d, J = 2.4 Hz, 1H), 3.87 (s, 3H), 2.87 (t, J = 7.4 Hz, 2H), 2.71 (t, J = 7.5 Hz, 2H), 2.03-1.86 (m, 2H), 1.50 (s, 9H). | m/z 470.4 (M + H)+ (ES+); 468.2 (M − H)− (ES−) | 469.56 |
| | 1-(tert-Butyl)-N-((5-(2-methoxypyridin-4- | | | |
| | yl)-2,3-dihydro-1H-inden-4- | | | |
| | yl)carbamoyl)-1H-pyrazole-3- | | | |
| | sulfonamide, sodium salt | | | |
| 253 | 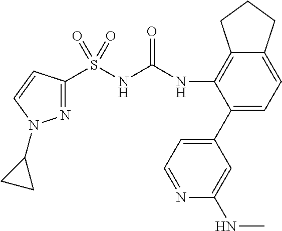 [SMILES: CNc1cc(-c2ccc3c(c2NC(=O)NS(=O)(=O)c2ccn(C4CC4)n2)CCC3)ccn1] | 1H NMR (DMSO-d6) δ 7.91 (d, J = 5.2 Hz, 1H), 7.78 (s, 1H), 7.38 (s, 1H), 7.09 (d, J = 7.2 Hz, 1H), 7.00 (d, J = 7.4 Hz, 1H), 6.48-6.37 (m, 3H), 6.35 (s, 1H), 3.86-3.66 (m, 1H), 2.88 (t, J = 7.4 Hz, 2H), 2.78 (d, J = 4.7 Hz, 3H), 2.72-2.62 (m, 2H), 1.95 (p, J = 7.5 Hz, 2H), 1.08-0.94 (m, 4H). One exchangeable proton not observed. | m/z 453.3 (M + H)+ (ES+) | 452.53 |
| | 1-Cyclopropyl-N-((5-(2- | | | |
| | (methylamino)pyridin-4-yl)-2,3-dihydro- | | | |
| | 1H-inden-4-yl)carbamoyl)-1H-pyrazole-3- | | | |
| | sulfonamide | | | |
| 254 | 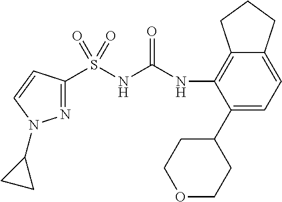 [SMILES: O=C(Nc1c(C2CCOCC2)ccc2c1CCC2)NS(=O)(=O)c1ccn(C2CC2)n1] 1-Cyclopropyl-N-((5-(tetrahydro-2H- | 1H NMR (DMSO-d6) δ 7.78 (s, 1H), 7.64 (s, 1H), 6.99 (d, J = 7.8 Hz, 1H), 6.95 (d, J = 7.8 Hz, 1H), 6.49-6.38 (m, 1H), 3.92- 3.86 (m, 2H), 3.77-3.71 (m, 1H), 3.35-3.28 (m, 2H), 2.98-2.90 (m, 1H), 2.81 (t, J = 7.4 Hz, 2H), 2.64 (t, J = 7.4 Hz, 2H), 1.91 (p, J = 7.5 Hz, 2H), 1.56-1.47 (m, 4H), 1.05- 0.95 (m, 4H). Acidic NH not observed. | m/z 431.8 (M + H)+ (ES+) | 430.52 |
| | pyran-4-yl)-2,3-dihydro-1H-inden-4- | | | |
| | yl)carbamoyl)-1H-pyrazole-3-sulfonamide | | | |
| 255 | 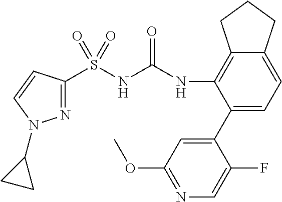 [SMILES: COc1cc(-c2ccc3c(c2NC(=O)NS(=O)(=O)c2ccn(C4CC4)n2)CCC3)c(F)cn1] 1-Cyclopropyl-N-((5-(5-fluoro-2- | 1H NMR (DMSO-d6) δ 8.09 (d, J = 1.5 Hz, 1H), 7.66 (d, J = 2.3 Hz, 1H), 7.44 (s, 1H), 7.07 (d, J = 7.6 Hz, 1H), 7.00 (d, J = 7.6 Hz, 1H), 6.70 (d, J = 5.0 Hz, 1H), 6.19 (d, J = 2.3 Hz, 1H), 3.84 (s, 3H), 3.70 (tt, J = 7.4, 3.9 Hz, 1H), 2.89 (t, J = 7.4 Hz, 2H), 2.74 (t, J = 7.5 Hz, 2H), 1.96 (p, J = 7.5 Hz, 2H), 1.06-0.99 (m, 2H), 0.99-0.92 (m, 2H). | m/z 472.3 (M + H)+ (ES+) | 471.50 |
| | methoxypyridin-4-yl)-2,3-dihydro-1H- | | | |
| | inden-4-yl)carbamoyl)-1H-pyrazole-3- | | | |
| | sulfonamide, sodium salt | | | |
| 256 | 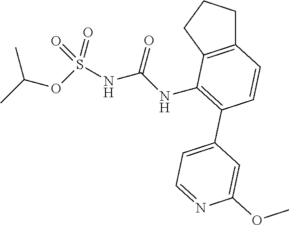 [SMILES: COc1cc(-c2ccc3c(c2NC(=O)NS(=O)(=O)OC(C)C)CCC3)ccn1] | 1H NMR (DMSO-d6) δ 8.10 (d, J = 5.3, 1H), 7.38 (br s, 1H), 7.09 (d, J = 7.6 Hz, 1H), 7.04 (d, J = 7.6 Hz, 1H), 6.98 (dd, J = 5.3, 1.4 Hz, 1H), 6.79 (d, J = 1.4 Hz, 1H), 4.38 (hept, J = 6.4 Hz, 1H), 3.85 (s, 3H), 2.90 (t, J = 7.4 Hz, 2H), 2.83 (t, J = 7.4 Hz, 2H), 1.99 (p, J = 7.4 Hz, 2H), 1.11 (d, J = 6.4 Hz, 6H). | m/z 406.3 (M + H)+ (ES+); 404.1 (M − H)− (ES−) | 405.47 |
| | Isopropyl (5-(2-methoxypyridin-4-yl)-2,3- | | | |
| | dihydro-1H-inden-4- | | | |
| | yl)carbamoylsulfamate, sodium salt | | | |
| 257 | 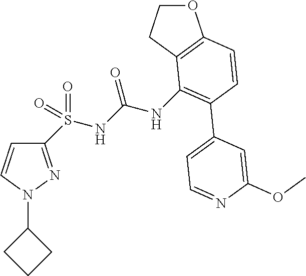 [SMILES: COc1cc(-c2ccc3c(c2NC(=O)NS(=O)(=O)c2ccn(C4CCC4)n2)CCO3)ccn1] | 1H NMR (DMSO-d6) δ 8.07-8.01 (m, 1H), 7.79- 7.71 (m, 1H), 7.42 (s, 1H), 7.01 (d, J = 8.2 Hz, 1H), 6.92-6.87 (m, 1H), 6.73 (s, 1H), 6.61 (d, J = 8.1 Hz, 1H), 6.38-6.31 (m, 1H), 4.82 (p, J = 8.5 Hz, 1H), 4.50 (t, J = 8.8 Hz, 2H), 3.86 (s, 3H), 3.05 (t, J = 8.7 Hz, 2H), 2.49- 2.40 (m, 2H), 2.39-2.30 (m, 2H), 1.82-1.70 (m, 2H). | m/z 470.3 (M + H)+ (ES+); 468.2 (M − H)− (ES−) | 469.51 |
| | 1-Cyclobutyl-N-((5-(2-methoxypyridin-4- | | | |
| | yl)-2,3-dihydrobenzofuran-4- | | | |
| | yl)carbamoyl)-1H-pyrazole-3- | | | |
| | sulfonamide, sodium salt | | | |
| 258 | 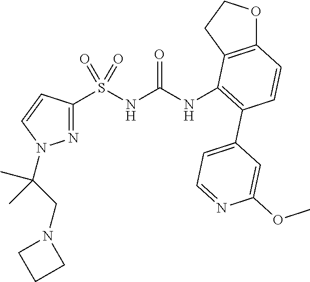 [SMILES: COc1cc(-c2ccc3c(c2NC(=O)NS(=O)(=O)c2ccn(C(C)(C)CN4CCC4)n2)CCO3)ccn1] | 1H NMR (DMSO-d6) δ 8.06 (t, J = 5.6 Hz, 1H), 7.71-7.63 (m, 1H), 7.47- 7.36 (m, 1H), 7.01 (d, J = 8.1 Hz, 1H), 6.95-6.88 (m, 1H), 6.74 (d, J = 1.5 Hz, 1H), 6.60 (dd, J = 8.2, 3.8 Hz, 1H), 6.36-6.29 (m, 1H), 4.49 (t, J = 8.8 Hz, 2H), 3.87 (s, 3H), 3.04 (t, J = 8.7 Hz, 2H), 2.93 (t, J = 7.0, 2.5 Hz, 4H), 2.64 (s, 2H), 1.80 (p, J = 6.8 Hz, 2H), 1.44 (s, 6H). | m/z 527.4 (M + H)+ (ES+); 525.1 (M − H)− (ES−) | 526.61 |
| | 1-(1-(Azetidin-1-yl)-2-rnethylpropan-2- | | | |
| | yl)-N-((5-(2-methoxypyridin-4-yl)-2,3- | | | |
| | dihydrobenzofuran-4-yl)carbamoyl)-1H- | | | |
| | pyrazole-3-sulfonamide, sodium salt | | | |
| 259 | 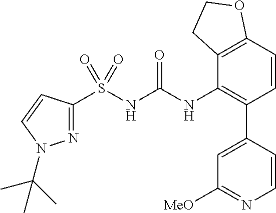 [SMILES: COc1cc(-c2ccc3c(c2NC(=O)NS(=O)(=O)c2ccn(C(C)(C)C)n2)CCO3)ccn1] | 1H NMR (DMSO-d6) δ 8.04 (dd, J = 5.3, 0.7 Hz, 1H), 7.74 (d, J = 2.4 Hz, 1H), 7.42 (s, 1H), 7.01 (d, J = 8.2 Hz, 1H), 6.89 (dd, J = 5.3, 1.5 Hz, 1H), 6.73- 6.73 (m, 1H), 6.60 (d, J = 8.2 Hz, 1H), 6.34 (d, J = 2.4 Hz, 1H), 4.48 (t, J = 8.8 Hz, 2H), 3.85 (s, 3H), 3.02 (t, J = 8.8 Hz, 2H), 1.50 (s, 9H). | m/z 472.1 (M + H)+ (ES+) | 471.53 |
| | 1-(tert-Butyl)-N-((5-(2-methoxypyridin-4- | | | |
| | yl)-2,3-dihydrobenzofuran-4- | | | |
| | yl)carbamoyl)-1H-pyrazole-3- | | | |
| | sulfonamide, sodium salt | | | |
| 260 | 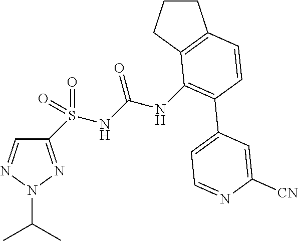 [SMILES: CC(C)n1ncc(S(=O)(=O)NC(=O)Nc2c(-c3ccnc(C#N)c3)ccc3c2CCC3)n1] | 1H NMR (DMSO-d6) δ 8.59 (dd, J = 5.1, 0.8 Hz, 1H), 7.92 (d, J = 1.9 Hz 1H), 7.65 (br s, 1H), 7.6 (dd, J = 5.1, 1.7 Hz, 1H), 7.55 (s, 1H), 7.12 (s, 2H), 4.78 (hept, J = 6.7 Hz, 1H), 2.90 (t, J = 7.5 Hz, 2H), 2.75 (t, J = 7.5 Hz, 2H), 1.97 (p, J = 7.5 Hz, 2H), 1.48 (d, J = 6.7 Hz, 6H). | m/z 452.1 (M + H)+ (ES+); 450.1 (M − H)− (ES−) | 451.50 |
| | N-((5-(2-Cyanopyridin-4-yl)-2,3-dihydro- | | | |
| | 1H-inden-4-yl)carbamoyl)-2-isopropyl- | | | |
| | 2H-1,2,3-triazole-4-sulfonamide, sodium | | | |
| | salt | | | |
| 261 | 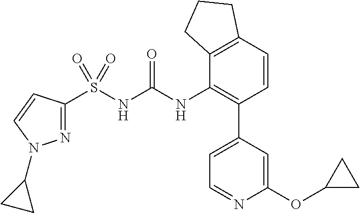 [SMILES: O=C(Nc1c(-c2ccnc(OC3CC3)c2)ccc2c1CCC2)NS(=O)(=O)c1ccn(C2CC2)n1] N-((5-(2-Cyclopropoxypyridin-4-yl)-2,3- dihydro-1H-inden-4-yl)carbamoyl)-1- cyclopropyl-1H-pyrazole-3-sulfonamide, sodium salt | 1H NMR (DMSO-d6) δ 8.07 (d, J = 5.2 Hz, 1H), 7.70 (d, J = 2.3 Hz, 1H), 7.37 (s, 1H), 7.08 (d, J = 7.7 Hz, 1H), 7.04 (d, J = 7.6 Hz, 1H), 6.95 (dd, J = 5.3, 1.4 Hz, 1H), 6.84 (d, J = 1.3 Hz, 1H), 6.29 (d, J = 2.3 Hz, 1H), 4.18 (tt, J = 6.3, 3.0 Hz, 1H), 3.72 (tt, J = 7.4, 3.9 Hz, 1H), 2.88 (t, J = 7.4 Hz, 2H), 2.73 (t, J = 7.5 Hz, 2H), 1.95 (p, J = 7.5 Hz, 2H), 1.09-1.01 (m, 2H), 0.95 (td, J = 7.4, 4.9 Hz, 2H), 0.79-0.72 (m, 2H), 0.69-0.64 (m, 2H). | m/z 480.1 (M + H)+ (ES+) | 479.55 |
| 262 | 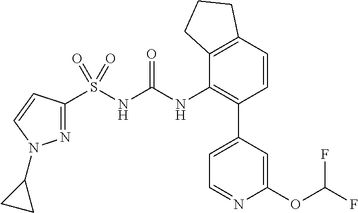 [SMILES: O=C(Nc1c(-c2ccnc(OC(F)F)c2)ccc2c1CCC2)NS(=O)(=O)c1ccn(C2CC2)n1] 1-Cyclopropyl-N-((5-(2- (difluoromethoxy)pyridin-4-yl)-2,3- | 1H NMR (DMSO-d6) δ 8.12 (d, J = 5.3 Hz, 1H), 7.73 (t, J = 73.1 Hz, 1H), 7.69-7.66 (m, 1H), 7.51 (s, 1H), 7.20 (dd, J = 5.4, 1.4 Hz, 1H), 7.09 (t, J = 5.7 Hz, 2H), 6.99 (d, J = 1.4 Hz, 1H), 6.26 (t, J = 2.3 Hz, 1H), 3.71 (tt, J = 7.5, 3.7 Hz, 1H), 2.90 (t, J = 7.4 Hz, 2H), 2.75 (t, J = 7.5 Hz, 2H), 1.97 (p, J = 7.5 Hz, 2H), 1.06-1.00 (m, 2H), 0.97-0.92 (m, 2H). | m/z 490.2 (M + H)+ (ES+); 488.0 (M − H)− (ES−) | 489.5 |
| | dihydro-1H-inden-4-yl)carbamoyl)-1H- | | | |
| | pyrazole-3-sulfonamide, sodium salt | | | |
| 263 | 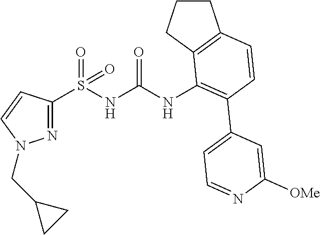 [SMILES: COc1cc(-c2ccc3c(c2NC(=O)NS(=O)(=O)c2ccn(CC4CC4)n2)CCC3)ccn1] 1-(Cyclopropylmethyl)-N-((5-(2- | 1H NMR (DMSO-d6) δ 8.02 (d, J = 5.3 Hz, 1H), 7.72 (d, J = 2.2 Hz, 1H), 7.37 (s, 1H), 7.08 (d, J = 7.7 Hz, 1H), 7.03 (d, J = 7.7 Hz, 1H), 6.90 (dd, J = 5.3, 1.4 Hz, 1H), 6.75 (d, J = 1.4 Hz, 1H), 6.32 (d, J = 2.2 Hz, 1H), 3.95 (d, J = 7.2 Hz, 2H), 3.86 (s, 3H), 2.87 (t, J = 7.5 Hz, 2H), 2.73 (t, J = 7.5 Hz, 2H), 1.94 (p, J = 7.5 Hz, 2H), 1.30-1.21 (m, 1H), 0.55- 0.47 (m, 2H), 0.40-0.32 (m, 2H). | m/z 468.1 (M + H)+ (ES+); 466.2 (M − H)− (ES−) | 467.54 |
| | methoxypyridin-4-yl)-2,3-dihydro-1H- | | | |
| | inden-4-yl)carbamoyl)-1H-pyrazole-3- | | | |
| | sulfonamide, sodium salt | | | |
| 264 | 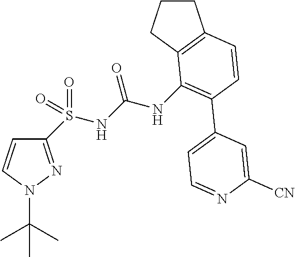 [SMILES: CC(C)(C)n1ccc(S(=O)(=O)NC(=O)Nc2c(-c3ccnc(C#N)c3)ccc3c2CCC3)n1] | 1H NMR (DMSO-d6) δ 8.58 (d, J = 5.1, 1H), 7.92 (d, J = 1.7, 1H), 7.73 (d, J = 2.3 Hz, 1H), 7.70 (s, 1H), 7.62 (dd, J = 5.1, 1.7, 1H), 7.13 (s, 2H), 6.26 (d, J = 2.3 Hz, 1H), 2.90 (t, J = 7.5 Hz, 2H), 2.75 (t, J = 7.5 Hz, 2H), 1.97 (p, J = 7.5 Hz, 2H), 1.50 (s, 9H). | m/z 465.1 (M + H)+ (ES+); 463.1 (M − H)− (ES−) | 464.54 |
| | 1-(tert-Butyl)-N-((5-(2-cyanopyridin-4- | | | |
| | yl)-2,3-dihydro-1H-inden-4- | | | |
| | yl)carbamoyl)-1H-pyrazole-3- | | | |
| | sulfonamide, sodium salt | | | |
| 265 | 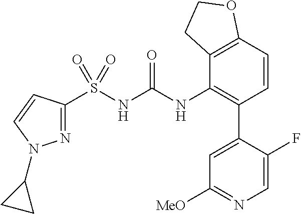 [SMILES: COc1cc(-c2ccc3c(c2NC(=O)NS(=O)(=O)c2ccn(C4CC4)n2)CCO3)c(F)cn1] 1-Cyclopropyl-N-((5-(5-fluoro-2- | 1H NMR (DMSO-d6) δ 8.09 (d, J = 1.6 Hz, 1H), 7.65 (d, J = 2.3 Hz, 1H), 7.49 (s, 1H), 6.97 (dd, J = 8.2, 1.4 Hz, 1H), 6.66 (d, J = 5.1 Hz, 1H), 6.59 (d, J = 8.2 Hz, 1H), 6.23 (d, J = 2.3 Hz, 1H), 4.51 (t, J = 8.8 Hz, 2H), 3.83 (s, 3H), 3.73-3.65 (m,1H), 3.07 (t, J = 8.8 Hz, 2H), 1.04- 0.98 (m, 2H), 0.98-0.91 (m, 2H). | m/z 474.1 (M + H)+ (ES+) | 473.48 |
| | methoxypyridin-4-yl)-2,3- | | | |
| | dihydrobenzofuran-4-yl)carbamoyl)-1H- | | | |
| | pyrazole-3-sulfonamide, sodium salt | | | |
| 266 | 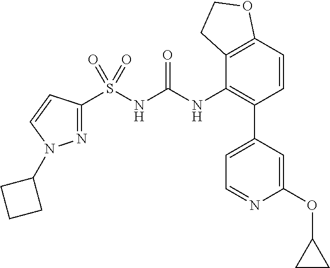 [SMILES: O=C(Nc1c(-c2ccnc(OC3CC3)c2)ccc2c1CCO2)NS(=O)(=O)c1ccn(C2CCC2)n1] | 1H NMR (DMSO-d6) δ 8.06 (d, J = 5.2 Hz, 1H), 7.74 (d, J = 2.3 Hz, 1H), 7.42 (s, 1H), 7.02 (d, J = 8.2 Hz, 1H), 6.92 (dd, J = 5.2, 1.5 Hz, 1H), 6.81 (s, 1H), 6.60 (d, J = 8.2 Hz, 1H), 6.33 (d, J = 2.3 Hz, 1H), 4.89-4.75 (m, 1H), 4.49 (t, J = 8.8 Hz, 2H), 4.26-4.09 (m, 1H), 3.04 (t, J = 8.8 Hz, 2H), 2.48- 2.40 (m, 2H), 2.38-2.28 (m, 2H), 1.83-1.69 (m, 2H), 0.78-0.71 (m, 2H), 0.71-0.63 (m, 2H). | m/z 496.1 (M + H)+ (ES+) | 495.55 |
| | 1-Cyclobutyl-N-((5-(2- | | | |
| | cyclopropoxypyridin-4-yl)-2,3- | | | |
| | dihydrobenzofuran-4-yl)carbamoyl)-1H- | | | |
| | pyrazole-3-sulfonamide, sodium salt | | | |
| 267 | 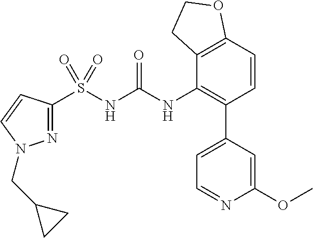 [SMILES: COc1cc(-c2ccc3c(c2NC(=O)NS(=O)(=O)c2ccn(CC4CC4)n2)CCO3)ccn1] 1-(Cyclopropylmethyl)-N-((5-(2- | 1H NMR (DMSO-d6) δ 8.03 (d, J = 5.3 Hz, 1H), 7.70 (d, J = 2.3 Hz, 1H), 7.42 (s, 1H), 7.01 (d, J = 8.2 Hz, 1H), 6.88 (dd, J = 5.3, 1.5 Hz, 1H), 6.72 (d, J = 1.4 Hz, 1H), 6.60 (d, J = 8.2 Hz, 1H), 6.33 (d, J = 2.2 Hz, 1H), 4.49 (t, J = 8.8 Hz, 2H), 3.94 (d, J = 7.2 Hz, 2H), 3.85 (s, 3H), 3.04 (t, J = 8.8 Hz, 2H), 1.29-1.18 (m, 1H), 0.56- 0.46 (m, 2H), 0.41-0.32 (m, 2H). | m/z 470.1 (M + H)+ (ES+) | 469.51 |
| | methoxypyridin-4-yl)-2,3- | | | |
| | dihydrobenzofuran-4-yl)carbamoyl)-1H- | | | |
| | pyrazole-3-sulfonamide, sodium salt | | | |
| 268 | 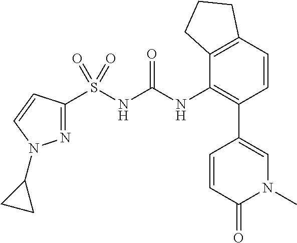 [SMILES: Cn1cc(-c2ccc3c(c2NC(=O)NS(=O)(=O)c2ccn(C4CC4)n2)CCC3)ccc1=O] 1-Cyclopropyl-N-((5-(1-methyl-6-oxo-1,6- | 1H NMR (DMSO-d6) δ 7.88 (d, J = 2.4 Hz, 1H), 7.78 (s, 1H), 7.63 (d, J = 2.6 Hz, 1H), 7.31 (dd, J = 9.4, 2.7 Hz, 1H), 7.13 (d, J = 7.6 Hz, 1H), 7.05 (d, J = 7.6 Hz, 1H), 6.58-6.51 (m, 1H), 6.33 (d, J = 9.3 Hz, 1H), 3.85-3.78 (m, 1H), 3.44 (s, 3H), 2.88 (t, J = 7.4 Hz, 2H), 2.66 (t, J = 7.5 Hz, 2H), 1.97 (p, J = 7.5 Hz, 2H), 1.12-0.96 (m, 4H). Acidic proton not observed | m/z 454.1 (M + H)+ (ES+) | 453.51 |
| | dihydropyridin-3-yl)-2,3-dihydro-1H- | | | |
| | inden-4-yl)carbamoyl)-1H-pyrazole-3- | | | |
| | sulfonamide | | | |
| 269 | 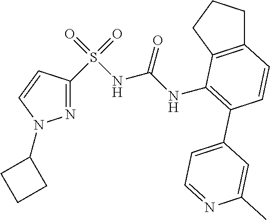 [SMILES: Cc1cc(-c2ccc3c(c2NC(=O)NS(=O)(=O)c2ccn(C4CCC4)n2)CCC3)ccn1] 1-Cyclobutyl-N-((5-(2-methylpyridin-4- yl)-2,3-dihydro-1H-inden-4- | 1H NMR (DMSO-d6) δ 8.31 (d, J = 5.2 Hz, 1H), 7.74 (d, J = 2.3 Hz, 1H), 7.32 (s, 1H), 7.19 (s, 1H), 7.12 (dd, J = 5.3, 1.7 Hz, 1H), 7.08 (d, J = 7.7 Hz, 1H), 7.03 (d, J = 7.6 Hz, 1H), 6.31 (d, J = 2.3 Hz, 1H), 4.82 (p, J = 8.5 Hz, 1H), 2.88 (t, J = 7.4 Hz, 2H), 2.74 (t, J = 7.5 Hz, 2H), 2.49-2.45 (m, 2H), 2.43 (s, 3H), 2.39-2.31 (m, 2H), 1.95 (p, J = 7.5 Hz, 2H), 1.82-1.71 (m, 2H). | m/z 452.1 (M + H)+ (ES+); 450.0 (M − H)− (ES−) | 451.54 |
| | yl)carbamoyl)-1H-pyrazole-3- | | | |
| | sulfonamide, sodium salt | | | |
| 270 | 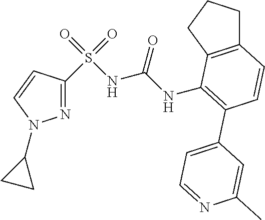 [SMILES: Cc1cc(-c2ccc3c(c2NC(=O)NS(=O)(=O)c2ccn(C4CC4)n2)CCC3)ccn1] 1-Cyclopropyl-N-((5-(2-methylpyridin-4- | 1H NMR (DMSO-d6) δ 8.33 (d, J = 5.2 Hz, 1H), 7.68 (d, J = 2.2 Hz, 1H), 7.28 (s, 1H), 7.20 (s, 1H), 7.13 (d, J = 4.0 Hz, 1H), 7.08 (d, J = 7.7 Hz, 1H), 7.03 (d, J = 7.6 Hz, 1H), 6.27 (d, J = 2.3 Hz, 1H), 3.73-3.65 (m, 1H), 2.89 (t, J = 7.4 Hz, 2H), 2.75 (t, J = 7.4 Hz, 2H), 2.44 (s, 3H), 1.96 (p, J = 7.5 Hz, 2H), 1.07-0.99 (m, 2H), 0.99-0.91 (m, 2H). | m/z 438.2 (M + H)+ (ES+); 436.1 (M − H)− (ES−) | 437.51 |
| | yl)-2,3-dihydro-1H-inden-4- | | | |
| | yl)carbamoyl)-1H-pyrazole-3- | | | |
| | sulfonamide, sodium salt | | | |
| 271 | 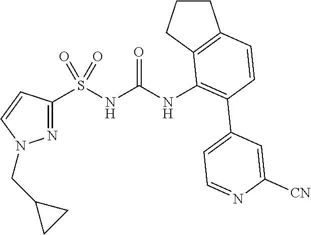 [SMILES: N#Cc1cc(-c2ccc3c(c2NC(=O)NS(=O)(=O)c2ccn(CC4CC4)n2)CCC3)ccn1] N-((5-(2-Cyanopyridin-4-yl)-2,3-dihydro- | 1H NMR (DMSO-d6) δ 11.0 (br s, 1H), 8.65 (d, J = 5.1 Hz, 1H), 8.05 (br s, 1H), 7.93 (d, J = 1.6 Hz, 1H), 7.82 (s, 1H), 7.62 (dd, J = 5.1, 1.6 Hz, 1H), 7.23 (d, J = 7.7 Hz, 1H), 7.18 (d, J = 7.7 Hz, 1H), 6.47 (s, 1H), 4.02 (d, J = 7.2 Hz, 2H), 2.93 (t, J = 7.5 Hz, 2H), 2.74 (t, J = 7.5 Hz, 2H), 2.01 (p, J = 7.5 Hz, 2H), 1.29-1.21 (m, 1H), 0.59-0.52 (m, 2H), 0.40-0.37 (m, 2H). | m/z 463.1 (M + H)+ (ES+); 461.1 (M − H)− (ES−) | 462.52 |
| | 1H-inden-4-yl)carbamoyl)-1- | | | |
| | (cyclopropylmethyl)-1H-pyrazole-3- | | | |
| | sulfonamide | | | |
| 272 | 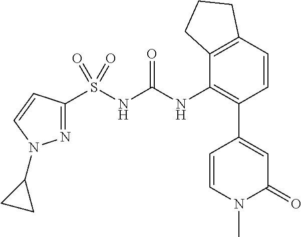 [SMILES: Cn1ccc(-c2ccc3c(c2NC(=O)NS(=O)(=O)c2ccn(C4CC4)n2)CCC3)cc1=O] 1-Cyclopropyl-N-((5-(1-methyl-2-oxo-1,2- | 1H NMR (DMSO-d6) δ 7.69 (d, J = 2.3 Hz, 1H), 7.52 (d, J = 7.0 Hz, 1H), 7.36 (s, 1H), 7.05 (d, J = 7.7 Hz, 1H), 7.00 (d, J = 7.7 Hz, 1H), 6.32 (d, J = 2.3 Hz, 1H), 6.29 (d, J = 1.9 Hz, 1H), 6.18 (dd, J = 7.0, 2.0 Hz, 1H), 3.72 (tt, J = 7.5, 3.8 Hz, 1H), 3.43 (s, 3H), 2.87 (t, J = 7.4 Hz, 2H), 2.70 (t, J = 7.4 Hz, 2H), 1.94 (p, J = 7.5 Hz, 2H), 1.07-1.01 (m, 2H), 1.00-0.92 (m, 2H). | m/z 454.2 (M + H)+ (ES+) | 453.51 |
| | dihydropyridin-4-yl)-2,3-dihydro-1H- | | | |
| | inden-4-yl)carbamoyl)-1H-pyrazole-3- | | | |
| | sulfonamide, sodium salt | | | |
| 273 | 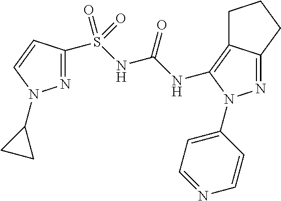 [SMILES: O=C(Nc1c2c(nn1-c1ccncc1)CCC2)NS(=O)(=O)c1ccn(C2CC2)n1] | 1H NMR (DMSO-d6) δ 11.59 (s, 1H), 8.63 (s, 1H), 8.55-8.45 (m, 2H), 7.88 (s, 1H), 7.61-7.45 (m, 2H), 6.57 (s, 1H), 3.85- 3.77 (m, 1H), 2.64 (dd, J = 9.1, 5.7 Hz, 2H), 2.30 (p, J = 7.4 Hz, 2H), 1.14-0.91 (m, 4H). 2H Obscured by DMSO peak, | m/z 414.1 (M + H)+ (ES+) | 413.45 |
| | 1-Cyclopropyl-N-((2-(pyridin-4-yl)- | | | |
| | 2,4,5,6-tetrahydrocyclopenta[c]pyrazol-3- | | | |
| | yl)carbamoyl)-1H-pyrazole-3-sulfonamide | | | |
| 274 | 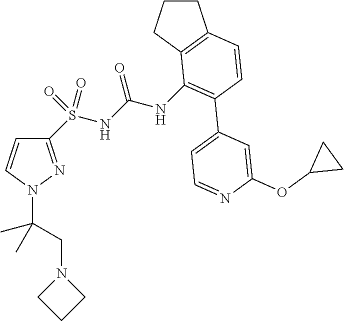 [SMILES: CC(C)(CN1CCC1)n1ccc(S(=O)(=O)NC(=O)Nc2c(-c3ccnc(OC4CC4)c3)ccc3c2CCC3)n1] | 1H NMR (DMSO-d6) δ 8.09 (d, J = 5.2 Hz, 1H), 7.68 (d, J = 2.4 Hz, 1H), 7.38 (s, 1H), 7.07 (d, J = 7.7 Hz, 1H), 7.04 (d, J = 7.7 Hz, 1H), 6.97 (dd, J = 5.3, 1.4 Hz, 1H), 6.86 (s, 1H), 6.30 (d, J = 2.3 Hz, 1H), 4.20 (tt, J = 6.3, 3.1 Hz, 1H), 2.95 (t, J = 7.0 Hz, 4H), 2.87 (t, J = 7.4 Hz, 2H), 2.71 (t, J = 7.5 Hz, 2H), 2.65 (s, 2H), 1.93 (p, J = 7.5 Hz, 2H), 1.82 (p, J = 7.0 Hz, 2H), 1.45 (s, 6H), 0.80-0.73 (m, 2H), 0.72-0.65 (m, 2H). | m/z 551.1 (M + H)+ (ES+) | 550.67 |
| | 1-(1-(Azetidin-1-yl)-2-methylpropan-2- | | | |
| | yl)-N-((5-(2-cyclopropoxypyridin-4-yl)- | | | |
| | 2,3-dihydro-1H-inden-4-yl)carbamoyl)- | | | |
| | 1H-pyrazole-3-sulfonamide, sodium salt | | | |
| 275 | 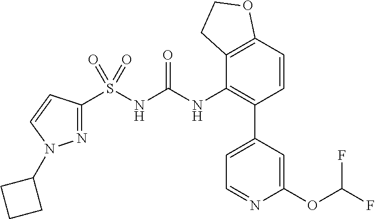 [SMILES: O=C(Nc1c(-c2ccnc(OC(F)F)c2)ccc2c1CCO2)NS(=O)(=O)c1ccn(C2CCC2)n1] 1-Cyclobutyl-N-((5-(2- | 1H NMR (DMSO-d6) δ 8.11 (d, J = 5.3 Hz, 1H), 7.96-7.51 (m, 3H), 7.17 (dd, J = 5.3, 1.5 1H), 7.07 (d, J = 8.2 Hz, 1H), 6.97 (d, J = 1.3 Hz, 1H), 6.63 (d, J = 8.2 Hz, 1H), 6.31 (d, J = 2.3 Hz, 1H), 4.81 (p, J = 8.4 Hz, 1H), 4.51 (t, J = 8.8 Hz, 2H), 3.06 (t, J = 8.8 Hz, 2H), 2.48-2.39 (m, 2H), 2.38- 2.29 (m, 2H), 1.81-1.71 (m, 2H). | m/z 506.0 (M + H)+ (ES+) | 505.49 |
| | (difluoromethoxy)pyridin-4-yl)-2,3- | | | |
| | dihydrobenzofuran-4-yl)carbamoyl)-1H- | | | |
| | pyrazole-3-sulfonamide, sodium salt | | | |
| 276 | 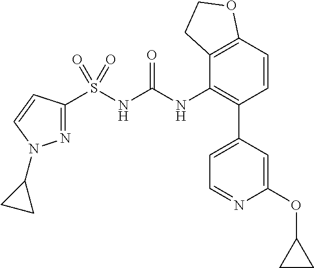 [SMILES: O=C(Nc1c(-c2ccnc(OC3CC3)c2)ccc2c1CCO2)NS(=O)(=O)c1ccn(C2CC2)n1] | 1H NMR (DMSO-d6) δ 8.07 (d, J = 5.3 Hz, 1H), 7.68 (d, J = 2.3 Hz, 1H), 7.40 (s, 1H), 7.02 (d, J = 8.2 Hz, 1H), 6.93 (dd, J = 5.3, 1.4 Hz, 1H), 6.81 (d, J = 1.3 Hz, 1H), 6.60 (d, J = 8.2 Hz, 1H), 6.30 (d, J = 2.3 Hz, 1H), 4.50 (t, J = 8.8 Hz, 2H), 4.22-4.12 (m, 1H), 3.78-3.65 (m, 1H), 3.06 (t, J = 8.8 Hz, 2H), 1.06-0.98 (m, 2H), 0.98-0.91 (m, 2H), 0.78- 0.71 (m, 2H), 0.71-0.64 (m, 2H). | m/z 482.1 (M + H)+ (ES+) | 481.52 |
| | N-((5-(2-Cyclopropoxypyridin-4-yl)-2,3- | | | |
| | dihydrobenzofuran-4-yl)carbamoyl)-1- | | | |
| | cyclopropyl-1H-pyrazole-3-sulfonamide, | | | |
| | sodium salt | | | |
| 277 | 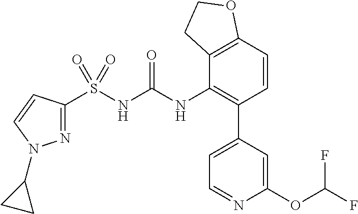 [SMILES: O=C(Nc1c(-c2ccnc(OC(F)F)c2)ccc2c1CCO2)NS(=O)(=O)c1ccn(C2CC2)n1] 1-Cyclopropyl-N-((5-(2- | 1H NMR (DMSO-d6) δ 8.12 (d, J = 5.3 Hz, 1H), 7.94-7.50 (m, 3H), 7.18 (dd, J = 5.3, 1.5 Hz, 1H), 7.07 (d, J = 8.2 Hz, 1H), 6.96 (d, J = 1.3 Hz, 1H), 6.63 (d, J = 8.2 Hz, 1H), 6.28 (d, J = 2.3 Hz, 1H), 4.52 (t, J = 8.8 Hz, 2H), 3.79-3.64 (m, 1H), 3.08 (t, J = 8.7 Hz, 2H), 1.05- 0.98 (m, 2H), 0.97-0.91 (m, 2H). | m/z 492.1 (M + H)+ (ES+) | 491.47 |
| | (difluoromethoxy)pyridin-4-yl)-2,3- | | | |
| | dihydrobenzofuran-4-yl)carbamoyl)-1H- | | | |
| | pyrazole-3-sulfonamide, sodium salt | | | |
| 278 | 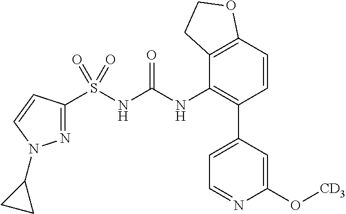 [SMILES: [2H]C([2H])([2H])Oc1cc(-c2ccc3c(c2NC(=O)NS(=O)(=O)c2ccn(C4CC4)n2)CCO3)ccn1] 1-Cyclopropyl-N-((5-(2-(methoxy- | 1H NMR (DMSO-d6) δ 8.04 (d, J = 5.3 Hz, 1H), 7.68 (d, J = 2.3 Hz, 1H), 7.39 (s, 1H), 7.01 (d, J = 8.2 Hz, 1H), 6.89 (dd, J = 5.3, 1.4 Hz, 1H), 6.72 (d, J = 1.3 Hz, 1H), 6.60 (d, J = 8.2 Hz, 1H), 6.30 (d, J = 2.3 Hz, 1H), 4.50 (t, J = 8.8 Hz, 2H), 3.80-3.64 (m, 1H), 3.05 (t, J = 8.7 Hz, 2H), 1.06-0.97 (m, 2H), 1.00-0.90 (m, 2H). | m/z 459.2 (M + H)+ (ES+) | 458.51 |
| | d3)pyridin-4-yl)-2,3-dihydrobenzofuran- | | | |
| | 4-yl)carbamoyl)-1H-pyrazole-3- | | | |
| | sulfonamide, sodium salt | | | |
| 279 | 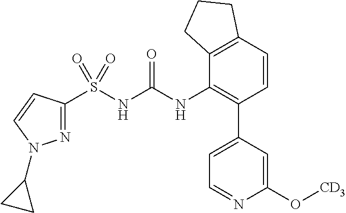 [SMILES: [2H]C([2H])([2H])Oc1cc(-c2ccc3c(c2NC(=O)NS(=O)(=O)c2ccn(C4CC4)n2)CCC3)ccn1] 1-Cyclopropyl-N-((5-(2-(methoxy- | 1H NMR (DMSO-d6) δ 8.03 (d, J = 5.3 Hz, 1H), 7.71 (d, J = 2.3 Hz, 1H), 7.36 (s, 1H), 7.08 (d, J = 7.7 Hz, 1H), 7.03 (d, J = 7.6 Hz, 1H), 6.91 (dd, J = 5.3, 1.3 Hz, 1H), 6.75 (d, J = 1.3 Hz, 1H), 6.29 (d, J = 2.3 Hz, 1H), 3.72 (tt, J = 7.4, 3.9 Hz, 1H), 2.88 (t, J = 7.4 Hz, 2H), 2.73 (t, J = 7.5 Hz, 2H), 1.95 (p, J = 7.5 Hz, 2H), 1.07-0.92 (m, 4H). | m/z 457.2 (M + H)+ (ES+) | 456.53 |
| | d3)pyridin-4-yl)-2,3-dihydro-1H-inden-4- | | | |
| | yl)carbamoyl)-1H-pyrazole-3- | | | |
| | sulfonamide, sodium salt | | | |
| 280 | 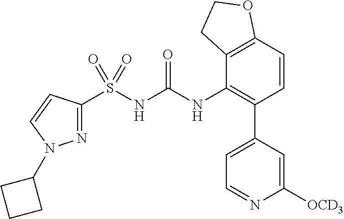 1-Cyclobutyl-N-((5-(2-(methoxy- | 1H NMR (DMSO-d6) δ 8.05 (d, J = 5.3 Hz, 1H), 7.70 (d, J = 2.3 Hz, 1H), 7.35 (s, 1H), 7.00 (d, J = 8.2 Hz, 1H), 6.91 (dd, J = 5.3, 1.4 Hz, 1H), 6.73 (d, J = 1.3 Hz, 1H), 6.59 (d, J = 8.2 Hz, 1H), 6.31 (d, J = 2.3 Hz, 1H), 4.80 (p, J = 8.5 Hz, 1H), 4.49 (t, J = 8.8 Hz, 2H), 3.05 (t, J = 8.8 Hz, 2H), 2.47-2.39 (m, 2H), 2.38-2.30 (m, 2H), 1.82-1.71 (m, 2H). | m/z 472.8 (M + H)+ (ES+); 471.0 (M − H)− (ES−) | 472.53 |
| | d3)pyridin-4-yl)-2,3-dihydrobenzofuran- | | | |
| | 4-yl)carbamoyl)-1H-pyrazole-3- | | | |
| | sulfonamide, sodium salt | | | |
| 281 | 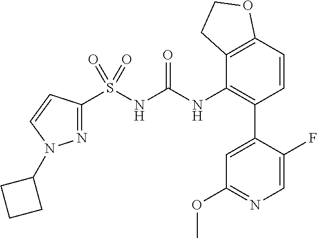 [SMILES: COc1cc(-c2ccc3c(c2NC(=O)NS(=O)(=O)c2ccn(C4CCC4)n2)CCO3)c(F)cn1] | 1H NMR (DMSO-d6) δ 8.09 (d, J = 1.7 Hz, 1H), 7.66 (d, J = 2.2 Hz, 1H), 7.45 (s, 1H), 6.99-6.89 (m, 1H), 6.68 (d, J = 5.0 Hz, 1H), 6.59 (d, J = 8.1 Hz, 1H), 6.22 (s, 1H), 4.78 (p, J = 8.4 Hz, 1H), 4.50 (t, J = 8.8 Hz, 2H), 3.84 (s, 3H), 3.06 (t, J = 8.8 Hz, 2H), 2.46-2.38 (m, 2H), 2.38-2.29 (m, 2H), 1.88-1.70 (m, 2H). | m/z 488.1 (M + H)+ (ES+) | 487.50 |
| | 1-Cyclobutyl-N-((5-(5-fluoro-2- | | | |
| | methoxypyridin-4-yl)-2,3- | | | |
| | dihydrobenzofuran-4-yl)carbamoyl)-1H- | | | |
| | pyrazole-3-sulfonamide, sodium salt | | | |
| 282 | 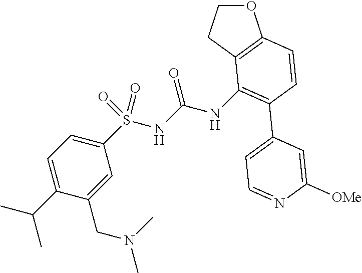 [SMILES: COc1cc(-c2ccc3c(c2NC(=O)NS(=O)(=O)c2ccc(C(C)C)c(CN(C)C)c2)CCO3)ccn1] 3-((Dimethylamino)methyl)-4-isopropyl- | 1H NMR (DMSO-d6) δ 8.02 (d, J = 5.1 Hz, 1H), 7.55 (d, J = 2.0 Hz, 1H), 7.53 (dd, J = 8.1, 2.1 HZ, 1H), 7.27 (d, J = 8.1 Hz, 1H), 7.23 (br s,1H), 7.06 (d, J = 7.7 Hz, 1H), 7.02 (d, J = 7.7 Hz, 1H), 6.90 (dd, J = 5.1, 1.3 Hz, 1H), 6.76 (t, J = 1.3 Hz, 1H), 3.87 (s, 3H), 3.38 (sept, J = 6.9 Hz, 1H), 3.37 (s, 2H), 2.86 (t, J = 7.5 Hz, 2H), 2.67 (t, J = 7.5 Hz, 2H), 2.13 (s, 6H), 1.91 (p, J = 7.5 Hz, 2H), 1.18 (d, J = 6.8 Hz, 6H). | m/z 523.1 (M + H)+ (ES+); 521.0 (M − H)− (ES−) | 522.66 |
| | N-((5-(2-methoxypyridin-4-yl)-2,3- | | | |
| | dihydro-1H-inden-4-yl)carbamoyl) | | | |
| | benzenesulfonamide, sodium salt | | | |
| 283 | 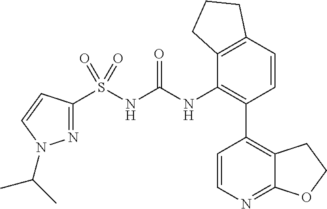 [SMILES: CC(C)n1ccc(S(=O)(=O)NC(=O)Nc2c(-c3ccnc4c3CCO4)ccc3c2CCC3)n1] N-((5-(2,3-Dihydrofuro[2,3-b]pyridin-4- yl)-2,3-dihydro-1H-inden-4- yl)carbamoyl)-1-isopropyl-1H-pyrazole-3- | 1H NMR (DMSO-d6) δ 7.89 (d, J = 2.3 Hz, 1H), 7.83 (d, J = 5.3 Hz, 1H), 7.77 (s, 1H), 7.17 (d, J = 7.6 Hz, 1H), 7.07 (d, J = 7.6 Hz, 1H), 6.60 (dd, J = 5.4, 1.1 Hz, 1H), 6.53 (s, 1H), 4.58 (sept, J = 6.7 Hz, 1H), 4.46 (t, J = 8.5 Hz, 2H), 3.01 (t, J = 8.5 Hz, 2H), 2.91 (t, J = 7.4 Hz, 2H), 2.67 (t, J = 7.5 Hz, 2H), 1.97 (p, J = 7.5 Hz, 2H), 1.43 (d, J = 6.7 Hz, 6H). Acidic NH not observed. | m/z 468.1 (M + H)+ (ES+) | 467.54 |
| | sulfonamide | | | |
| 284 | 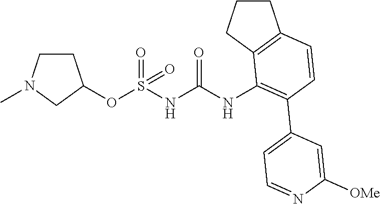 [SMILES: COc1cc(-c2ccc3c(c2NC(=O)NS(=O)(=O)OC2CCN(C)C2)CCC3)ccn1] 1-Methylpyrrolidin-3-yl (5-(2- methoxypyridin-4-yl)-2,3-dihydro-1H- | 1H NMR (DMSO-d6) δ 9.92 (br s, 1H), 8.13 (d, J = 5.3 Hz, 1H), 7.51 (br s, 1H), 7.12 (d, J = 7.7 Hz, 1H), 7.07 (d, J = 7.7 Hz, 1H), 6.99 (dd, J = 5.3, 1.3 Hz, 1H), 6.80 (d, J = 1.3 Hz, 1H), 4.99-4.91 (m, 1H), 3.87 (s, 3H), 3.49 (d, J = 12.3 Hz, 1H), 3.20- 3.13 (m, 3H), 2.91 (t, J = 7.4 Hz, 2H), 2.83 (t, J = 7.4 Hz, 2H), 2.76 (s, 3H), 2.26-2.13 (m, 1H), 2.09- 1.95 (m, 3H). | m/z 447.2 (M + H)+ (ES+); 445.1 (M − H)− (ES−) | 446.52 |
| | inden-4-yl)carbamoylsulfamate | | | |
| 285 | 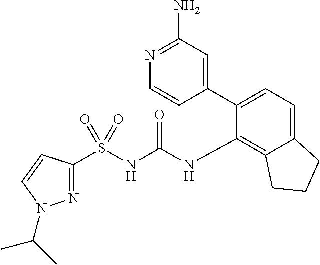 [SMILES: CC(C)n1ccc(S(=O)(=O)NC(=O)Nc2c(-c3ccnc(N)c3)ccc3c2CCC3)n1] | 1H NMR (DMSO-d6) δ 10.86 (s, 1H), 7.96 (d, J = 2.4 Hz, 1H), 7.84 (d, J = 5.2 Hz, 1H), 7.78 (s, 1H), 7.18 (d, J = 7.6 Hz, 1H), 7.03 (d, J = 7.6 Hz, 1H), 6.65 (d, J = 2.4 Hz, 1H), 6.37-6.33 (m, 2H), 5.98 (s, 2H), 4.61 (sept, J = 6.7 Hz, 1H), 2.89 (t, J = 7.4 Hz, 2H), 2.58 (t, J = 7.5 Hz, 2H), 1.95 (p, J = 7.5 Hz, 2H), 1.44 (d, J = 6.7 Hz, 6H). | m/z 441.2 (M + H)+ (ES+); 439.5 (M − H)− (ES−) | 440.52 |
| | N-((5-(2-Aminopyridin-4-yl)-2,3-dihydro- | | | |
| | 1H-inden-4-yl)carbamoyl)-1-isopropyl- | | | |
| | 1H-pyrazole-3-sulfonamide | | | |
| 286 | 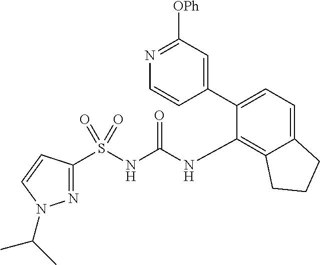 [SMILES: CC(C)n1ccc(S(=O)(=O)NC(=O)Nc2c(-c3ccnc(Oc4ccccc4)c3)ccc3c2CCC3)n1] | 1H NMR (DMSO-d6) δ 8.03-7.97 (m, 1H), 7.69- 7.64 (m, 1H), 7.51-7.39 (m, 3H), 7.20 (t, J = 7.4 Hz, 1H), 7.16 (d, J = 7.5 Hz, 2H), 7.11-7.03 (m, 3H), 6.98 (s, 1H), 6.40- 6.24 (m, 1H), 4.47 (sept, J = 6.6 Hz, 1H), 2.89 (t, J = 7.4 Hz, 2H), 2.73 (t, J = 7.4 Hz, 2H), 1.95 (p, J = 7.5 Hz, 2H), 1.39 (d, J = 6.7 Hz, 6H). | m/z 518.1 (M + H)+ (ES+); 516.4 (M − H)− (ES−) | 517.6 |
| | 1-Isopropyl-N-((5-(2-phenoxypyridin-4- | | | |
| | yl)-2,3-dihydro-1H-inden-4- | | | |
| | yl)carbamoyl)-1H-pyrazole-3- | | | |
| | sulfonamide, sodium salt | | | |
| 287 | 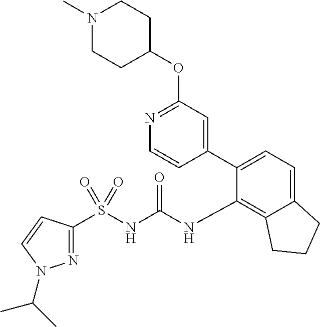 [SMILES: CC(C)n1ccc(S(=O)(=O)NC(=O)Nc2c(-c3ccnc(OC4CCN(C)CC4)c3)ccc3c2CCC3)n1] | 1H NMR (DMSO-d6) δ 7.99 (d, J = 5.3 Hz, 1H), 7.69 (s, 1H), 7.37 (s, 1H), 7.06 (d, J = 7.7 Hz, 1H), 7.02 (d, J = 7.6 Hz, 1H), 6.87 (d, J = 5.2 Hz, 1H), 6.69 (s, 1H), 6.31 (s, 1H), 5.03-4.93 (m, 1H), 4.49 (sept, J = 6.8 Hz, 1H), 2.87 (t, J = 7.5 Hz, 2H), 2.75-2.61 (m, 4H), 2.19 (s, 3H), 2.14 (t, J = 10.8 Hz, 2H), 2.03-1.97 (m, 2H), 1.93 (p, J = 7.5 Hz, 2H), 1.73-1.64 (m, 2H), 1.40 (d, J = 6.7 Hz, 6H). | m/z 539.2 (M + H)+ (ES+); 537.3 (M − H)− (ES−) | 538.66 |
| | 1-Isopropyl-N-((5-(2-((1-methylpiperidin- | | | |
| | 4-yl)oxy)pyridin-4-yl)-2,3-dihydro-1H- | | | |
| | inden-4-yl)carbamoyl)-1H-pyrazole-3- | | | |
| | sulfonamide, sodium salt | | | |
| 288 | 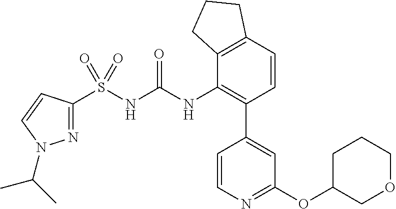 [SMILES: CC(C)n1ccc(S(=O)(=O)NC(=O)Nc2c(-c3ccnc(OC4CCCOC4)c3)ccc3c2CCC3)n1] 1-Isopropyl-N-((5-(2-((tetrahydro-2H- pyran-3-yl)oxy)pyridin-4-yl)-2,3-dihydro- 1H-inden-4-yl)carbamoyl)-1H-pyrazole-3- sulfonamide, sodium salt | 1H NMR (DMSO-d6) δ 8.00 (d, J = 5.3 Hz, 1H), 7.74 (s, 1H), 7.44 (s, 1H), 7.08 (d, J = 8.2 Hz, 1H), 7.04 (d, J = 7.4 Hz, 1H), 6.88 (d, J = 5.3 Hz, 1H), 6.71 (d, J = 1.3 Hz, 1H), 6.35 (s, 1H), 5.05-4.99 (m, 1H), 4.51 (p, J = 6.4 Hz, 1H), 3.90 (dd, J = 11.2, 2.3 Hz, 1H), 3.69- 3.63 (m, 1H), 3.56-3.46 (m, 2H), 2.87 (t, J = 7.4 Hz, 2H), 2.69 (t, J = 7.5 Hz, 2H), 2.11-2.03 (m, 1H), 1.93 (p, J = 7.5 Hz, 2H), 1.84-1.71 (m, 2H), 1.61-1.52 (m, 1H), 1.40 | m/z 526.1 (M + H)+ (ES+) | 525.62 |
| | | (d, J = 6.6 Hz, 6H). | | |
| 289 | 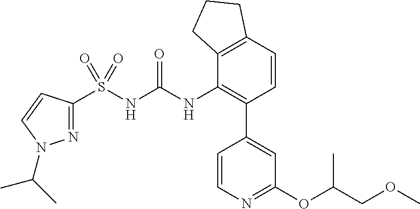 [SMILES: COCC(C)Oc1cc(-c2ccc3c(c2NC(=O)NS(=O)(=O)c2ccn(C(C)C)n2)CCC3)ccn1] 1-Isopropyl-N-((5-(2-((1-methoxypropan- 2-yl)oxy)pyridin-4-yl)-2,3-dihydro-1H- inden-4-yl)carbamoyl)-1H-pyrazole-3- sulfonamide, sodium salt | 1H NMR (DMSO-d6) δ 7.98 (dd, J = 5.3, 0.7 Hz, 1H), 7.72 (d, J = 2.3 Hz, 1H), 7.40 (s, 1H), 7.07 (d, J = 7.7 Hz, 1H), 7.02 (d, J = 7.6 Hz, 1H), 6.86 (dd, J = 5.3, 1.5 Hz, 1H), 6.71- 6.67 (m, 1H), 6.33 (d, J = 2.3 Hz, 1H), 5.38-5.29 (m, 1H), 4.51 (sept, J = 6.7 Hz, 1H), 3.56 (dd, J = 10.3, 6.0 Hz, 1H), 3.46 (dd, J = 10.3, 4.3 Hz, 1H), 3.30 (s, 3H), 2.87 (t, J = 7.4 Hz, 2H), 2.69 (t, J = 7.5 Hz, 2H),1.93 (p, J = 7.5 Hz, 2H), 1.40 (d, J = 6.7 Hz, 6H), 1.27 (d, J = | m/z 514.1 (M + H)+ (ES+) | 513.61 |
| | | 6.3 Hz, 3H). | | |
| 290 | 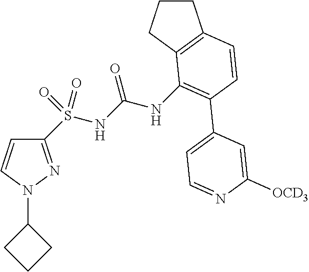 | 1H NMR (DMSO-d6) δ 8.02 (dd, J = 5.3, 0.7 Hz, 1H), 7.76 (d, J = 2.3 Hz, 1H), 7.36 (s, 1H), 7.07 (d, J = 7.7 Hz, 1H), 7.03 (d, J = 7.6 Hz, 1H), 6.91 (dd, J = 5.3, 1.4 Hz, 1H), 6.78- 6.73 (m, 1H), 6.32 (d, J = 2.3 Hz, 1H), 4.88-4.76 (m, 1H), 2.88 (t, J = 7.4 Hz, 2H), 2.72 (t, J = 7.4 Hz, 2H), 2.50-2.42 (m, 2H), 2.39-2.30 (m, 2H), 1.94 (p, J = 7.5 Hz, 2H), 1.81-1.71 (m, 2H). | m/z 471.2 (M + H)+ (ES+) | 470.56 |
| | 1-Cyclobutyl-N-((5-(2-(methoxy- | | | |
| | d3)pyridin-4-yl)-2,3-dihydro-1H-inden-4- | | | |
| | yl)carbamoyl)-1H-pyrazole-3- | | | |
| | sulfonamide, sodium salt | | | |
| 291 | 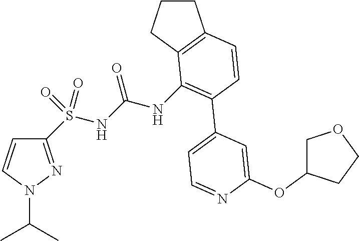 [SMILES: CC(C)n1ccc(S(=O)(=O)NC(=O)Nc2c(-c3ccnc(OC4CCOC4)c3)ccc3c2CCC3)n1] 1-Isopropyl-N-((5-(2-((tetrahydrofuran-3- yl)oxy)pyridin-4-yl)-2,3-dihydro-1H- inden-4-yl)carbamoyl)-1H-pyrazole-3- sulfonamide, sodium salt | 1H NMR (DMSO-d6) δ 8.02 (d, J = 5.3 Hz, 1H), 7.73 (d, J = 2.3 Hz, 1H), 7.44 (s, 1H), 7.08 (d, J = 7.7 Hz, 1H), 7.04 (d, J = 7.6 Hz, 1H), 6.90 (dd, J = 5.3, 1.5 Hz, 1H), 6.77- 6.70 (m, 1H), 6.34 (s, 1H), 5.54-5.48 (m, 1H), 4.51 (sept, J = 6.8 Hz, 1H), 3.94 (dd, J = 10.2,4.8 Hz, 1H), 3.90-3.83 (m, 1H), 3.82-3.73 (m, 2H), 2.88 (t, J = 7.4 Hz, 2H), 2.69 (t, J = 7.5 Hz, 2H), 2.29- 2.20 (m, 1H), 2.07-1.99 (m, 1H), 1.94 (p, J = 7.5 Hz, 2H), 1.40 (d, J = 6.7 Hz, 6H). | m/z 512.1 (M + H)+ (ES+) | 511.59 |
| 292 | 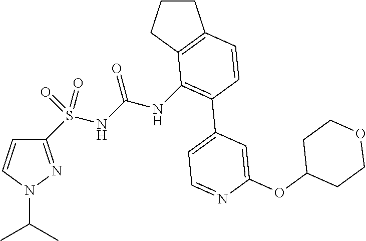 [SMILES: CC(C)n1ccc(S(=O)(=O)NC(=O)Nc2c(-c3ccnc(OC4CCOCC4)c3)ccc3c2CCC3)n1] 1-Isopropyl-N-((5-(2-((tetrahydro-2H- pyran-4-yl)oxy)pyridin-4-yl)-2,3-dihydro- 1H-inden-4-yl)carbamoyl)-1H-pyrazole-3- sulfonamide, sodium salt | 1H NMR (DMSO-d6) δ 7.99 (d, J = 5.0 Hz, 1H), 7.73 (d, J = 2.3 Hz, 1H), 7.42 (s, 1H), 7.07 (d, J = 7.7 Hz, 1H), 7.03 (d, J = 7.6 Hz, 1H), 6.88 (dd, J = 5.3, 1.5 Hz, 1H), 6.74- 6.70 (m, 1H), 6.34 (d, J = 2.3 Hz, 1H), 5.20 (tt, J = 8.9, 4.1 Hz, 1H), 4.51 (sept, J = 6.7 Hz, 1H), 3.88 (dt, J = 11.7, 4.2 Hz, 2H), 3.56-3.46 (m, 2H), 2.87 (t, J = 7.4 Hz, 2H), 2.69 (t, J = 7.5 Hz, 2H), 2.07-2.00 (m, 2H), 1.93 (p, J = 7.5 Hz, 2H), 1.71- 1.59 (m, 2H), 1.40 (d, J = 6.7 Hz, 6H). | m/z 526.1 (M + H)+ (ES+) | 525.62 |
| 293 | 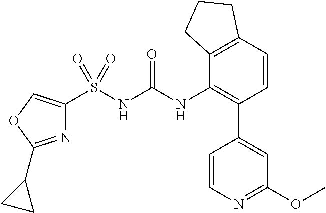 [SMILES: COc1cc(-c2ccc3c(c2NC(=O)NS(=O)(=O)c2coc(C4CC4)n2)CCC3)ccn1] 2-Cyclopropyl-N-((5-(2-methoxypyridin- | 1H NMR (DMSO-d6) δ 8.06 (d, J = 5.1 Hz, 1H), 7.81 (s, 1H), 7.38 (s, 1H), 7.09 (d, J = 7.7 Hz, 1H), 7.04 (d, J = 7.6 Hz, 1H), 6.95 (dd, J = 5.3, 1.5 Hz, 1H), 6.76 (d, J = 1.7 Hz, 1H), 3.86 (s, 3H), 2.89 (t, J = 7.5 Hz, 2H), 2.75 (t, J = 7.5 Hz, 2H), 2.13-2.05 (m, 1H), 1.97 (p, J = 7.5 Hz, 2H), 1.07-1.01 (m, 2H), 0.97-0.91 (m, 2H). | m/z 455.2 (M + H)+ (ES+) | 454.5 |
| | 4-yl)-2,3-dihydro-1H-inden-4- | | | |
| | yl)carbamoyl)oxazole-4-sulfonamide, | | | |
| | sodium salt | | | |
| 294 | 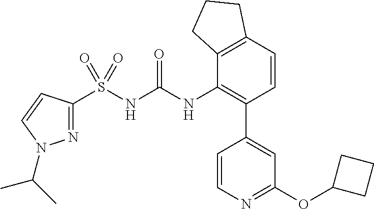 [SMILES: CC(C)n1ccc(S(=O)(=O)NC(=O)Nc2c(-c3ccnc(OC4CCC4)c3)ccc3c2CCC3)n1] N-((5-(2-Cyclobutoxypyridin-4-yl)-2,3- dihydro-1H-inden-4-yl)carbamoyl)-1- isopropyl-1H-pyrazole-3-sulfonamide, sodium salt | 1H NMR (DMSO-d6) δ 7.99 (d, J = 5.3 Hz, 1H), 7.73 (d, J = 2.3 Hz, 1H), 7.43 (s, 1H), 7.08 (d, J = 7.7 Hz, 1H), 7.03 (d, J = 7.6 Hz, 1H), 6.87 (dd, J = 5.3, 1.5 Hz, 1H), 6.70 (d, J = 1.3 Hz, 1H), 6.35 (s, 1H), 5.19-5.10 (m, 1H), 4.51 (sept, J = 6.6 Hz, 1H), 2.87 (t, J = 7.4 Hz, 2H), 2.69 (t, J = 7.5 Hz, 2H), 2.45-2.38 (m, 2H), 2.12- 2.03 (m, 2H), 1.93 (p, J = 7.5 Hz, 2H), 1.83-1.75 (m, 1H), 1.70-1.60 (m, 1H), 1.40 (d, J = 6.7 Hz, 6H). | m/z 496.2 (M + H)+ (ES+) | 495.59 |
| 295 | 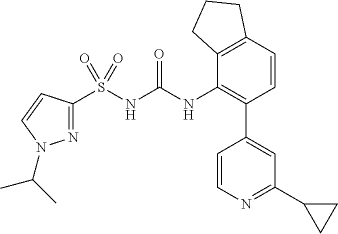 [SMILES: CC(C)n1ccc(S(=O)(=O)NC(=O)Nc2c(-c3ccnc(C4CC4)c3)ccc3c2CCC3)n1] N-((5-(2-Cyclopropylpyridin-4-yl)-2,3- | 1H NMR (DMSO-d6) δ 8.24 (d, J = 5.1 Hz, 1H), 7.72 (d, J = 2.3 Hz, 1H), 7.37 (s, 1H), 7.21 (dd, J = 1.7, 0.8 Hz, 1H), 7.09 (d, J = 7.7 Hz, 1H), 7.04 (d, J = 7.7 Hz, 1H), 7.02 (dd, J = 5.1, 1.7 Hz, 1H), 6.32 (d, J = 2.3 Hz, 1H), 4.50 (sept, J = 6.6 Hz, 1H), 2.88 (t, J = 7.5 Hz, 2H), 2.73 (t, J = 7.5 Hz, 2H), 2.06-1.88 (m, 3H), 1.40 (d, J = 6.7 Hz, 6H), 0.96-0.87 (m, 4H). | m/z 466.0 (M + H)+ (ES+) | 465.57 |
| | dihydro-1H-inden-4-yl)carbamoyl)-1- | | | |
| | isopropyl-1H-pyrazole-3-sulfonamide, | | | |
| | sodium salt | | | |
| 296 | 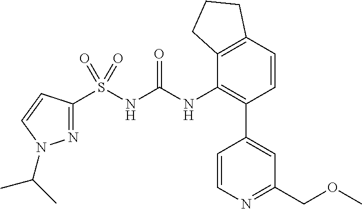 [SMILES: COCc1cc(-c2ccc3c(c2NC(=O)NS(=O)(=O)c2ccn(C(C)C)n2)CCC3)ccn1] 1-Isopropyl-N-((5-(2- (methoxymethyl)pyridin-4-yl)-2,3- | 1H NMR (DMSO-d6) δ 8.35 (dd, J = 5.1, 0.8 Hz, 1H), 7.72 (d, J = 2.3 Hz, 1H), 7.43 (s, 1H), 7.33 (dd, J = 1.7, 0.8 Hz, 1H), 7.21 (dd, J = 5.1, 1.7 Hz, 1H), 7.10 (d, J = 7.6 Hz, 1H), 7.04 (d, J = 7.6 Hz, 1H), 6.30 (d, J = 2.3 Hz, 1H), 4.55-4.46 (m, 3H), 3.30 (s, 3H), 2.89 (t, J = 7.5 Hz, 2H), 2.72 (t, J = 7.4 Hz, 2H), 1.95 (p, J = 7.5 Hz, 2H), 1.40 (d, J = 6.7 Hz, 6H). | m/z 470.2 (M + H)+ (ES+) | 469.56 |
| | dihydro-1H-inden-4-yl)carbamoyl)-1H- | | | |
| | pyrazole-3-sulfonamide, sodium salt | | | |
| 297 | 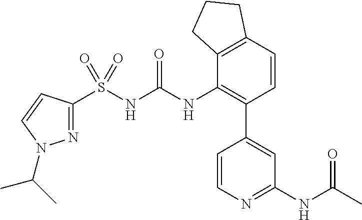 [SMILES: CC(=O)Nc1cc(-c2ccc3c(c2NC(=O)NS(=O)(=O)c2ccn(C(C)C)n2)CCC3)ccn1] | 1H NMR (DMSO-d6) δ 10.46 (s, 1H), 8.12 (d, J = 5.2 Hz, 1H), 8.04 (s, 1H), 7.73 (d, J = 2.3 Hz, 1H), 7.41 (s, 1H), 7.11 (d, J = 7.7 Hz, 1H), 7.05-6.94 (m, 2H), 6.33 (d, J = 2.4 Hz, 1H), 4.50 (sept, J = 6.7 Hz, 1H), 2.88 (t, J = 7.4 Hz, 2H), 2.70 (t, J = 7.5 Hz, 2H), 2.10 (s, 3H), 1.94 (p, J = 7.5 Hz, 2H), 1.41 (d, J = 6.7 Hz, 6H). | m/z 483.3 (M + H)+ (ES+) | 482.56 |
| | N-(4-(4-(3-((1-Isopropyl-1H-pyrazol-3- | | | |
| | yl)sulfonyl)ureido)-2,3-dihydro-1H- | | | |
| | inden-5-yl)pyridin-2-yl)acetamide, | | | |
| | sodium salt | | | |
| 298 | 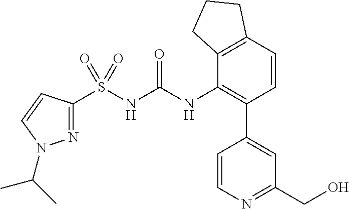 [SMILES: CC(C)n1ccc(S(=O)(=O)NC(=O)Nc2c(-c3ccnc(CO)c3)ccc3c2CCC3)n1] N-((5-(2-(Hydroxymethyl)pyridin-4-yl)- | 1H NMR (DMSO-d6) δ 8.40 (d, J = 5.0 Hz, 1H), 7.90 (s, 1H), 7.85 (s, 1H), 7.39 (d, J = 1.6 Hz, 1H), 7.22 (d, J = 7.6 Hz, 1H), 7.17-7.12 (m, 1H), 7.10 (d, J = 7.7 Hz, 1H), 6.56 (s, 1H), 5.38 (t, J = 5.8 Hz, 1H), 4.62-4.53 (m, 3H), 2.92 (t, J = 7.5 Hz, 2H), 2.68-2.59 (m, 2H), 1.98 (p, J = 7.5 Hz, 2H), 1.43 (d, J = 6.7 Hz, 6H). Acidic NH not observed. | m/z 456.3 (M + H)+ (ES+) | 455.53 |
| | 2,3-dihydro-1H-inden-4-yl)carbamoyl)-1- | | | |
| | isopropyl-1H-pyrazole-3-sulfonamide | | | |
| 299 | 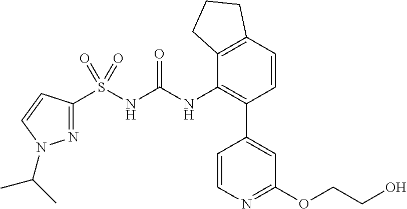 [SMILES: CC(C)n1ccc(S(=O)(=O)NC(=O)Nc2c(-c3ccnc(OCCO)c3)ccc3c2CCC3)n1] N-((5-(2-(2-Hydroxyethoxy)pyridin-4-yl)- 2,3-dihydro-1H-inden-4-yl)carbamoyl)-1- isopropyl-1H-pyrazole-3-sulfonamide | 1H NMR (DMSO-d6) δ 8.08 (d, J = 5.4 Hz, 1H), 7.86 (s, 1H), 7.75 (s, 1H), 7.17 (d, J = 7.8 Hz, 1H), 7.08 (d, J = 7.6 Hz, 1H), 6.87 (d, J = 5.3 Hz, 1H), 6.72 (s, 1H), 6.53 (s, 1H), 4.90-4.82 (m, 1H), 4.60- 4.52 (m, 1H), 4.30 (t, J = 5.2 Hz, 2H), 3.77-3.69 (m, 2H), 2.90 (t, J = 7.6 Hz, 2H), 2.68-2.60 (m, 2H), 1.96 (p, J = 7.4 Hz, 2H), 1.43 (d, J = 6.7 Hz, 6H). Acidic NH not observed. | m/z 486.1 (M + H)+ (ES+) | 485.56 |
| 300 | 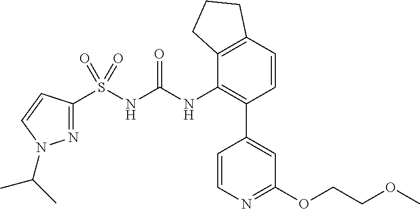 [SMILES: COCCOc1cc(-c2ccc3c(c2NC(=O)NS(=O)(=O)c2ccn(C(C)C)n2)CCC3)ccn1] 1-Isopropyl-N-((5-(2-(2- methoxyethoxy)pyridin-4-yl)-2,3- dihydro-1H-inden-4-yl)carbamoyl)-1H- | 1H NMR (DMSO-d6) δ 8.00 (dd, J = 5.3, 0.7 Hz, 1H), 7.71 (d, J = 2.3 Hz, 1H), 7.40 (s, 1H), 7.07 (d, J = 7.7 Hz, 1H), 7.03 (d, J = 7.6 Hz, 1H), 6.90 (dd, J = 5.3, 1.4 Hz, 1H), 6.75 (d, J = 1.2 Hz, 1H), 6.32 (d, J = 2.3 Hz, 1H), 4.50 (sept, J = 6.6 Hz, 1H), 4.41- 4.37 (m, 2H), 3.71-3.65 (m, 2H), 3.31 (s, 3H), 2.87 (t, J = 7.4 Hz, 2H), 2.69 (t, J = 7.5 Hz, 2H), 1.93 (p, J = 7.5 Hz, 2H), 1.40 (d, J = 6.7 Hz, 6H). | m/z 500.2 (M + H)+ (ES+) | 499.58 |
| | pyrazole-3-sulfonamide, sodium salt | | | |
| 301 | 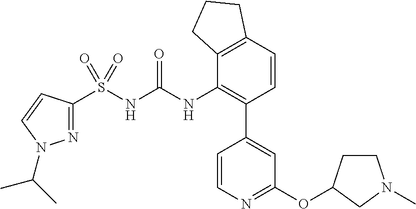 [SMILES: CC(C)n1ccc(S(=O)(=O)NC(=O)Nc2c(-c3ccnc(OC4CCN(C)C4)c3)ccc3c2CCC3)n1] 1-Isopropyl-N-((5-(2-((1- methylpyrrolidin-3-yl)oxy)pyridin-4-yl)- 2,3-dihydro-1H-inden-4-yl)carbamoyl)- 1H-pyrazole-3-sulfonamide, sodium salt | 1H NMR (DMSO-d6) δ 8.01 (d, J = 5.3 Hz, 1H), 7.73 (d, J = 2.3 Hz, 1H), 7.44 (s, 1H), 7.08 (d, J = 7.6 Hz, 1H), 7.03 (d, J = 7.6 Hz, 1H), 6.88 (dd, J = 5.3, 1.5 Hz, 1H), 6.71 (d, J = 1.4 Hz, 1H), 6.34 (d, J = 2.3 Hz, 1H), 5.41-5.35 (m, 1H), 4.51 (sept, J = 6.6 Hz, 1H), 2.91-2.84 (m, 3H), 2.77-2.67 (m, 4H), 2.43 (q, J = 7.6 Hz, 1H), 2.34-2.26 (m, 4H), 1.93 (p, J = 7.5 Hz, 2H), 1.89-1.81 (m, 1H), 1.40 (d, J = 6.7 Hz, 6H). | m/z 525.2 (M + H)+ (ES+) | 524.64 |
| 302 | 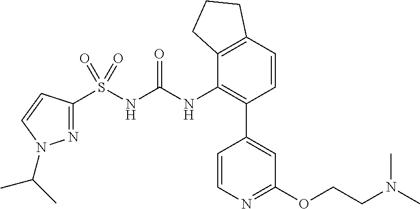 [SMILES: CC(C)n1ccc(S(=O)(=O)NC(=O)Nc2c(-c3ccnc(OCCN(C)C)c3)ccc3c2CCC3)n1] N-((5-(2-(2- (Dimethylamino)ethoxy)pyridin-4-yl)- | 1H NMR (DMSO-d6) δ 8.01 (d, J = 5.3 Hz, 1H), 7.73 (d, J = 2.3 Hz, 1H), 7.43 (s, 1H), 7.08 (d, J = 7.7 Hz, 1H), 7.03 (d, J = 7.6 Hz, 1H), 6.89 (dd, J = 5.3, 1.5 Hz, 1H), 6.73 (s, 1H), 6.34 (d, J = 2.3 Hz, 1H), 4.51 (sept, J = 6.7 Hz, 1H), 4.36 (t, J = 6.0 Hz, 2H), 2.87 (t, J = 7.4 Hz, 2H), 2.72-2.64 (m, 4H), 2.25 (s, 6H), 1.93 (p, J = 7.5 Hz, 2H), 1.40 (d, J = 6.7 Hz, 6H). | m/z 513.2 (M + H)+ (ES+) | 512.62 |
| | 2,3-dihydro-1H-inden-4-yl)carbamoyl)-1- | | | |
| | isopropyl-1H-pyrazole-3-sulfonamide, | | | |
| | sodium salt | | | |
| 303 | 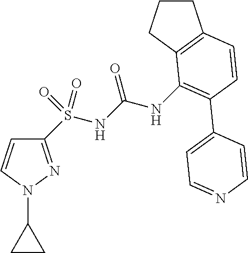 [SMILES: O=C(Nc1c(-c2ccncc2)ccc2c1CCC2)NS(=O)(=O)c1ccn(C2CC2)n1] | 1H NMR (DMSO-d6) δ 8.60-8.48 (m, 2H), 7.93 (s, 1H), 7.88 (br. s, 1H), 7.30-7.25 (m, 2H), 7.22 (d, J = 7.7 Hz, 1H), 7.12 (d, J = 7.6 Hz, 1H), 6.56 (s, 1H), 3.88-3.83 (m, 1H), 2.92 (t, J = 7.4 Hz, 2H), 2.76-2.60 (m, 2H), 2.00 (p, J = 7.5 Hz, 2H), 1.09-1.01 (m, 4H). One exchangeable proton not observed. | m/z 424.5 (M + H)+ (ES+) | 423.49 |
| | 1-Cyclopropyl-N-((5-(pyridin-4-yl)-2,3- | | | |
| | dihydro-1H-inden-4-yl)carbamoyl)-1H- | | | |
| | pyrazole-3-sulfonamide | | | |
| 304 | 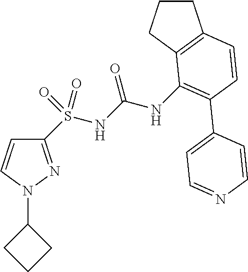 [SMILES: O=C(Nc1c(-c2ccncc2)ccc2c1CCC2)NS(=O)(=O)c1ccn(C2CCC2)n1] | 1H NMR (DMSO-d6) δ 8.54-8.49 (m, 2H), 7.97 (d, J = 2.4 Hz, 1H), 7.89 (s, 1H), 7.38-7.23 (m, 2H), 7.22 (d, J = 7.7 Hz, 1H), 7.12 (d, J = 7.7 Hz, 1H), 6.59 (s, 1H), 4.93 (p, J = 8.4 Hz, 1H), 2.91 (t, J = 7.4 Hz, 2H), 2.65 (t, J = 7.5 Hz, 2H), 2.47 (m, 4H), 1.98 (p, J = 7.4 Hz, 2H), 1.88-1.73 (m, 2H). Exchangeable proton not observed. | m/z 438.2 (M + H)+ (ES+) | 437.51 |
| | 1-Cyclobutyl-N-((5-(pyridin-4-yl)-2,3- | | | |
| | dihydro-1H-inden-4-yl)carbamoyl)-1H- | | | |
| | pyrazole-3-sulfonamide | | | |
| 305 | 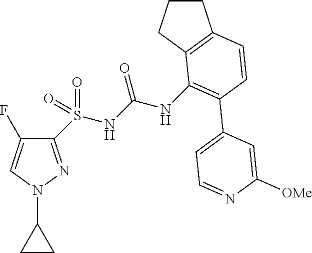 [SMILES: COc1cc(-c2ccc3c(c2NC(=O)NS(=O)(=O)c2nn(C4CC4)cc2F)CCC3)ccn1] | 1H NMR (DMSO-d6) δ 11.16 (s, 1H), 8.12 (d, J = 5.2 Hz, 1H), 8.09 (s, 1H), 7.80 (s, 1H), 7.19 (d, J = 7.7 Hz, 1H), 7.10 (d, J = 7.6 Hz, 1H), 6.88 (d, J = 5.3 Hz, 1H), 6.72 (s, 1H), 3.88 (s, 3H), 3.78 (br. s, 1H), 2.91 (t, J = 7.4 Hz, 2H), 2.67 (t, J = 7.5 Hz, 2H), 1.99 (p, J = 7.5 Hz, 2H), 1.11-0.93 (m, 4H). | m/z 472.1 (M + H)+ (ES+); 470.0 (M − H)− (ES−) | 471.5 |
| | 1-Cyclopropyl-4-fluoro-N-((5-(2- | | | |
| | methoxypyridin-4-yl)-2,3-dihydro-1H- | | | |
| | inden-4-yl)carbamoyl)-1H-pyrazole-3- | | | |
| | sulfonamide | | | |
| 306 | 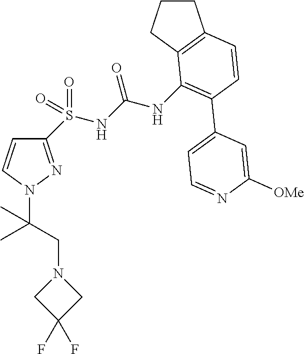 [SMILES: COc1cc(-c2ccc3c(c2NC(=O)NS(=O)(=O)c2ccn(C(C)(C)CN4CC(F)(F)C4)n2)CCC3)ccn1] | 1H NMR (DMSO-d6) δ 10.90 (br s, 1H), 8.14 (d, J = 5.3 Hz, 1H), 7.92 (br s, 2H), 7.19 (d, J = 7.7 Hz, 1H), 7.10 (d, J = 7.6 Hz, 1H), 6.89 (d, J = 5.1 Hz, 1H), 6.73 (s, 1H), 6.59 (br s, 1H), 3.88 (s, 3H), 2.93- 2.85 (m, 4H), 2.67-2.61 (m, 2H), 1.96 (p, J = 7.4 Hz, 2H), 1.51 (s, 6H). 4H obscured by solvent. | m/z 561.3 (M + H)+ (ES+) | 560.62 |
| | 1-(1-(3,3-Difluoroazetidin-1-yl)-2- | | | |
| | methylpropan-2-yl)-N-((5-(2- | | | |
| | methoxypyridin-4-yl)-2,3-dihydro-1H- | | | |
| | inden-4-yl)carbamoyl)-1H-pyrazole-3- | | | |
| | sulfonamide | | | |
| 307 | 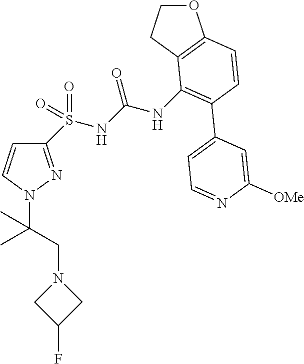 [SMILES: COc1cc(-c2ccc3c(c2NC(=O)NS(=O)(=O)c2ccn(C(C)(C)CN4CC(F)C4)n2)CCO3)ccn1] | 1H NMR (DMSO-d6) δ 8.13 (d, J = 5.3 Hz, 1H), 7.99-7.84 (m, 2H), 7.09 (d, J = 8.2 Hz, 1H), 6.87 (d, J = 5.3 Hz, 1H), 6.73 (d, J = 8.2 Hz, 1H), 6.70 (br s, 1H), 6.60 (br s, 1H), 4.96 (dp, J = 57.9, 5.2 Hz, 1H), 4.53 (t, J = 8.7 Hz, 2H), 2.78 (s, 3H), 3.01- 2.88 (m, 4H), 1.88 (s, 2H), 1.48 (s, 6H). Acidic NH not observed, 2H partially obscured by water peak. | m/z 545.2 (M + H)+ (ES+) | 544.60 |
| | 1-(1-(3-Fluoroazetidin-1-yl)-2- | | | |
| | methylpropan-2-yl)-N-((5-(2- | | | |
| | methoxypyridin-4-yl)-2,3- | | | |
| | dihydrobenzofuran-4-yl)carbamoyl)-1H- | | | |
| | pyrazole-3-sulfonamide | | | |
| 308 | 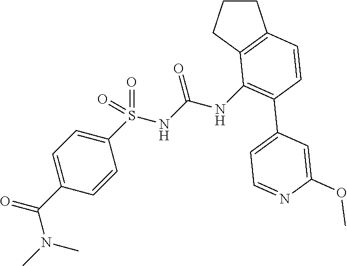 [SMILES: COc1cc(-c2ccc3c(c2NC(=O)NS(=O)(=O)c2ccc(C(=O)N(C)C)cc2)CCC3)ccn1] | 1H NMR (DMSO-d6) δ 8.03 (d, J = 5.3 Hz, 1H), 7.70 (d, J = 7.9 Hz, 2H), 7.36 (d, J = 7.9 Hz, 2H), 7.25 (s, 1H), 7.06 (d, J = 7.6 Hz, 1H), 7.01 (d, J = 7.6 Hz, 1H), 6.86 (d, J = 5.3 Hz, 1H), 6.73 (s, 1H), 3.86 (s, 3H), 3.00 (s, 3H), 2.91 (s, 3H), 2.87 (t, J = 7.4 Hz, 2H), 2.70 (t, J = 7.5 Hz, 2H), 1.93 (p, J = 7.5 Hz, 2H). | m/z 495.2 (M + H)+ (ES+); 493.4 (M − H)− (ES−) | 494.56 |
| | 4-(N-((5-(2Mmethoxypyridin-4-yl)-2,3- | | | |
| | dihydro-1H-inden-4- | | | |
| | yl)carbamoyl)sulfamoyl)-N,N- | | | |
| | dimethylbenzamide, sodium salt | | | |
| 309 | 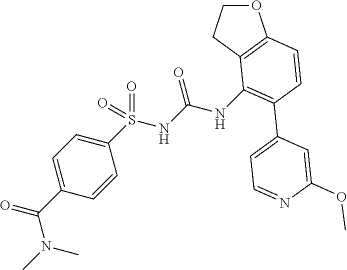 [SMILES: COc1cc(-c2ccc3c(c2NC(=O)NS(=O)(=O)c2ccc(C(=O)N(C)C)cc2)CCO3)ccn1] | 1H NMR (DMSO-d6) δ 8.04 (d, J = 5.3 Hz, 1H), 7.74 (d, J = 8.2 Hz, 2H), 7.38 (d, J = 8.2 Hz, 2H), 7.38 (s, 1H), 7.00 (d, J = 8.1 Hz, 1H), 6.83 (d, J = 5.3 Hz, 1H), 6.70 (s, 1H), 6.60 (d, J = 8.2 Hz, 1H), 4.48 (t, J = 8.7 Hz, 2H), 3.86 (s, 3H), 3.02 (t, J = 8.7 Hz, 2H), 3.00 (s, 3H), 2.90 (s, 3H). | m/z 497.1 (M + H)+ (ES+); 495.0 (M − H)− (ES−) | 496.54 |
| | 4-(N-((5-(2-Methoxypyridyl-4-yl)-2,3- | | | |
| | dihydrobenzofuran-4- | | | |
| | yl)carbamoyl)sulfamoyl)-N,N- | | | |
| | dimethylbenzamide, sodium salt | | | |
| 310 | 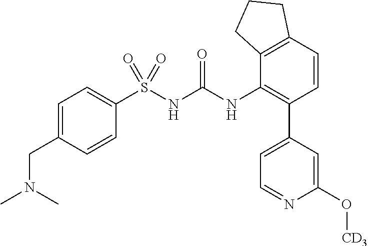 [SMILES: [2H]C([2H])([2H])Oc1cc(-c2ccc3c(c2NC(=O)NS(=O)(=O)c2ccc(CN(C)C)cc2)CCC3)ccn1] | 1H NMR (DMSO-d6) δ 10.52 (br s, 1H), 8.04 (d, J = 5.3 Hz, 1H), 7.80- 7.72 (m, 3H), 7.48 (d, J = 8.0 Hz, 2H), 7.16 (d, J = 7.9 Hz, 1H), 7.07 (d, J = 7.6 Hz, 1H), 6.76 (d, J = 5.2 Hz, 1H), 6.67 (s, 1H), 3.69 (br s, 2H), 2.88 (t, J = 7.4 Hz, 2H), 2.60 (t, J = 7.5 Hz, 2H), 2.31 (br s, 6H), 1.93 (p, J = 7.4 Hz, 2H). | m/z 484.3 (M + H)+ (ES+) | 483.6 |
| | 4-((Dimethylamino)methyl)-N-((5-(2- | | | |
| | (methoxy-d3)pyridin-4-yl)-2,3-dihydro- | | | |
| | 1H-inden-4- | | | |
| | yl)carbamoyl)benzenesulfonamide | | | |
| 311 | 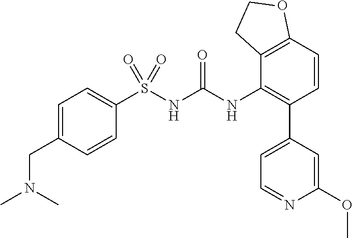 | 1H NMR (DMSO-d6) δ 8.04 (d, J = 5.3 Hz, 1H), 7.80-7.72 (m, 3H), 7.48 (d, J = 8.0 Hz, 2H), 7.04 (d, J = 8.2 Hz, 1H), 6.78- 6.73 (m, 1H), 6.71-6.63 (m, 2H), 4.50 (t, J = 8.7 Hz, 2H), 3.88 (s, 3H), 3.80 (s, 2H), 2.96 (t, J = 8.8 Hz, 2H), 2.38 (s, 6H). Acidic NH not observed. | m/z, 483.3 (M + H)+ (ES+) | 482.55 |
| | 4-((Dimethylamino)methyl)-N-((5-(2- | | | |
| | methoxypyridin-4-yl)-2,3- | | | |
| | dihydrobenzofuran-4- | | | |
| | yl)carbamoyl)benzenesulfonamide | | | |
| 312 | 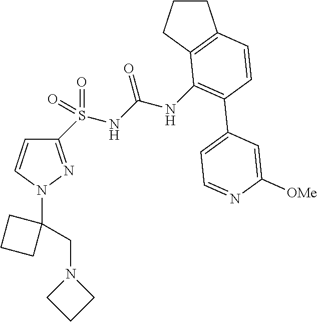 [SMILES: COc1cc(-c2ccc3c(c2NC(=O)NS(=O)(=O)c2ccn(C4(CN5CCC5)CCC4)n2)CCC3)ccn1] | 1H NMR (DMSO-d6) δ 8.13 (d, J = 5.3 Hz, 1H), 7.82 (s, 1H), 7.74 (br s, 1H), 7.16 (d, J = 7.7 Hz, 1H), 7.09 (d, J = 7.6 Hz, 1H), 6.90 (d, J = 5.3 Hz, 1H), 6.47 (s, 1H), 6.54 (br s, 1H), 3.88 (s, 3H), 3.08- 2.94 (m, 6H), 2.89 (t, J = 7.4 Hz, 2H), 2.66 (t, J = 7.6 Hz, 2H), 2.50-2.41 (m, 2H), 2.33-2.22 (m, 2H), 2.01-1.78 (m, 6H). Acidic NH not observed. | m/z 537.2 (M + H)+ (ES+); 535.1 (M − H)− (ES−) | 536.65 |
| | 1-(1-(Azetidin-1-ylmethyl)cyclobutyl)-N- | | | |
| | ((5-(2-methoxypyridin-4-yl)-2,3-dihydro- | | | |
| | 1H-inden-4-yl)carbamoyl)-1H-pyrazole-3- | | | |
| | sulfonamide | | | |
| 313 | 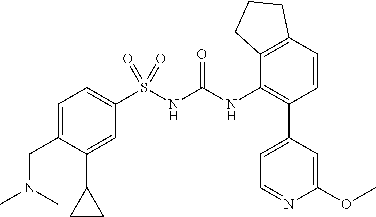 3-Cyclopropyl-4- ((dimethylamino)methyl)-N-((5-(2- | 1H NMR (DMSO-d6) δ 10.53 (s, 1H), 8.02 (d, J = 5.2 Hz, 1H), 7.89 (s, 1H), 7.63-7.57 1H), (m, 7.49 (d, J = 8.0 Hz, 1H), 7.46- 7.42 (m, 1H), 7.19 (d, J = 7.7 Hz, 1H), 7.09 (d, J = 7.6 Hz, 1H), 6.80-6.74 (m, 1H), 6.68 (s, 1H), 3.87 (s, 3H), 3.77 (s, 2H), 2.89 (t, J = 7.5 Hz, 2H), 2.31 (s, 6H), 2.27-2.18 (m, 1H), 1.98-1.88 (m, 2H), 1.06- 0.98 (m, 2H), 0.63-0.59 (m, 2H). 2H under DMSO peak. | m/z 521.2 (M + H)+ (ES+) | 520.64 |
| | methoxypyridin-4-yl)-2,3-dihydro-1H- | | | |
| | inden-4- | | | |
| | yl)carbamoyl)benzenesulfonamide | | | |
| 314 | 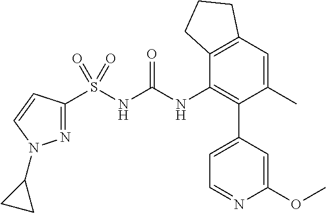 [SMILES: COc1cc(-c2c(C)cc3c(c2NC(=O)NS(=O)(=O)c2ccn(C4CC4)n2)CCC3)ccn1] | 1H NMR (DMSO-d6) δ 10.67 (s, 1H), 8.16 (d, J = 5.2 Hz, 1H), 7.96 (d, J = 2.4 Hz, 1H), 7.47 (s, 1H), 7.08 (s, 1H), 6.65 (d, J = 5.2 Hz, 1H), 6.61-6.47 (m, 2H), 3.93-3.83 (m, 4H), 2.87 (t, J = 7.5 Hz, 2H), 2.61-2.52 (m, 2H), 2.02-1.88 (m, H), 1.11- 0.98 (m, 4H). | m/z 468.3 (M + H)+ (ES+) | 467.54 |
| | 1-Cyclopropyl-N-((5-(2-methoxypyridin- | | | |
| | 4-yl)-6-methyl-2,3-dihydro-1H-inden-4- | | | |
| | yl)carbamoyl)-1H-pyrazole-3-sulfonamide | | | |
| 315 | 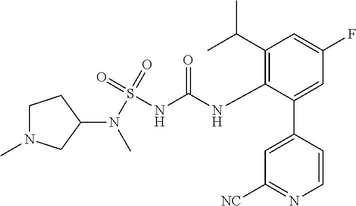 [SMILES: CC(C)c1cc(F)cc(-c2ccnc(C#N)c2)c1NC(=O)NS(=O)(=O)N(C)C1CCN(C)C1] | ¹H NMR (Methanol-d₄) δ 8.71 (dd, 1H), 7.97 (d, 1H), 7.73 (dd, 1H), 7.19 (dd, 1H), 7.02 (dd, 1H), 4.44 (q, 1H), 3.09-3.00 (m, 2H), 3.00-2.91 (m, 2H), 2.91-2.71 (m, 1H), 2.64 (s, 3H), 2.59 (s, 3H), 2.05 (dq, 1H), 1.98-1.82 (m, 1H), 1.35-1.16 (m, 6H). | m/z 475.2 (M + H)⁺ (ES⁺) | 474.56 |
| | 3-(N-Methyl-N-(1-methylpyrrolidin-3- | | | |
| | yl)sulfamoyl)-1-(5-(2-cyanopyridin-4-yl)- | | | |
| | 4-fluoro-6-isopropylphenyl)urea | | | |
| 316 | 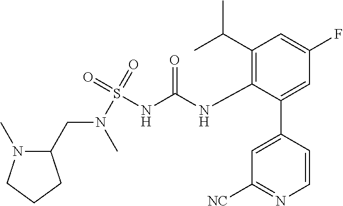 [SMILES: CC(C)c1cc(F)cc(-c2ccnc(C#N)c2)c1NC(=O)NS(=O)(=O)N(C)CC1CCCN1C] | ¹H NMR (Methanol-d₄) δ 8.73 (d, 1H), 8.03 (d, 1H), 7.78 (d, 1H), 7.21 (dd, 1H), 7.07 (dd, 1H), 4.57 (m, 1H), 3.37-3.07 (m, 3H), 2.91 (m, 2H), 2.79 (s, 3H), 2.65 (s, 3H), 2.22-1.83 (m, 4H), 1.25 (dd, 6H). | m/z 489.2 (M + H)⁺ (ES⁺) | 488.58 |
| | 3-(N-Methyl-N-((1-methylpyrrolidin-2- | | | |
| | yl)methyl)sulfamoyl)-1-(5-(2- | | | |
| | cyanopyridin-4-yl)-4-fluoro-6- | | | |
| | isopropylphenyl)urea | | | |
| 317 | 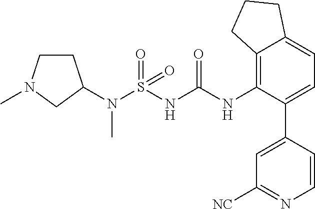 [SMILES: CN1CCC(N(C)S(=O)(=O)NC(=O)Nc2c(-c3ccnc(C#N)c3)ccc3c2CCC3)C1] | ¹H NMR (Methanol-d₄) δ 8.68 (dd, 1H), 7.95 (dd, 1H), 7.73 (dd, 1H), 7.32- 7.14 (m, 3H), 4.51 (q, 1H), 3.26-3.13 (m, 1H), 3.1- 2.89 (m, 6H), 2.70 (d, 6H), 2.14 (m, 3H), 2.09- 1.89 (m, 1H). | m/z 455.2 (M + H)⁺ (ES⁺) | 454.55 |
| | 3-(N-Methyl-N-(1-methylpyrrolidin-3- | | | |
| | yl)sulfamoyl)-1-(5-(2-cyanopyridin-4-yl)- | | | |
| | 2,3-dihydro-1H-inden-4-yl)urea | | | |
| 318 | 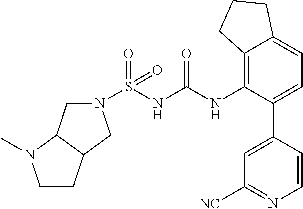 [SMILES: CN1CCC2CN(S(=O)(=O)NC(=O)Nc3c(-c4ccnc(C#N)c4)ccc4c3CCC4)CC21] | ¹H NMR (Methanol-d₄) δ 8.67 (dd, 1H), 7.95 (dd, 1H), 7.73 (dd, 1H), 7.21 (q, 2H), 3.62-3.40 (m, 2H), 3.16-2.88 (m, 9H), 2.81, (dt, 1H), 2.72 (s, 3H), 2.38-2.25 (m, 1H), 2.25- 2.04 (m, 2H), 1.57 (dd, 1H). | m/z 467.2 (M + H)⁺ (ES⁺) | 466.56 |
| | N-((5-(2-Cyanopyridin-4-yl)-2,3-dihydro- | | | |
| | 1H-inden-4-yl)carbamoyl)-1- | | | |
| | methylhexahydropyrrolo[3,4-b]pyrrole- | | | |
| | 5(1H)-sulfonamide | | | |
| 319 | 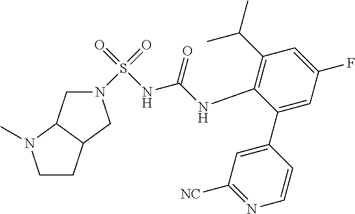 [SMILES: CC(C)c1cc(F)cc(-c2ccnc(C#N)c2)c1NC(=O)NS(=O)(=O)N1CC2CCN(C)C2C1] | ¹H NMR (Methanol-d₄) δ 8.71 (dd, 1H), 7.97 (d, 1H), 7.74 (dd, 1H), 7.19 (dd, 1H), 7.02 (dd, 1H), 3.46 (dd, 2H), 3.04-2.86 (m, 6H), 2.88-2.73 (m, 1H), 2.68 (s, 3H), 2.32-2.21 (m, 1H), 1.67-1.41 (m, 1H), 1.37-1.13 (m, 6H). | m/z 487.2 (M + H)⁺ (ES⁺) | 486.57 |
| | N-((2-(2-Cyanopyridin-4-yl)-4-fluoro-6- | | | |
| | isopropylphenyl)carbamoyl)-1- | | | |
| | methylhexahydropyrrolo[3,4-b]pyrrole- | | | |
| | 5(1H)-sulfonamide | | | |
| 320 | 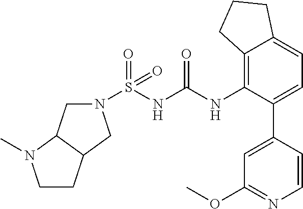 [SMILES: COc1cc(-c2ccc3c(c2NC(=O)NS(=O)(=O)N2CC4CCN(C)C4C2)CCC3)ccn1] | ¹H NMR (Methanol-d₄) δ 8.12 (dd, 1H), 7.27-7.10 (m, 2H), 7.02 (dd, 1H), 6.85 (d, 1H), 3.93 (s, 3H), 3.55 (dd, 2H), 3.18-3.05 (m, 3H), 2.96 (dt, 6H), 2.80 (dt, 1H), 2.72 (s, 3H), 2.30 (dt, 1H), 2.11 (p, 2H), 1.62 (ddd, 1H). | m/z 472.2 (M + H)⁺ (ES⁺) | 471.58 |
| | N-((5-(2-Methoxypyridin-4-yl)-2,3- | | | |
| | dihydro-1H-inden-4-yl)carbamoyl)-1- | | | |
| | methylhexahydropyrrolo[3,4-b]pyrrole- | | | |
| | 5(1H)-sulfonamide, potassium salt | | | |
| 321 | 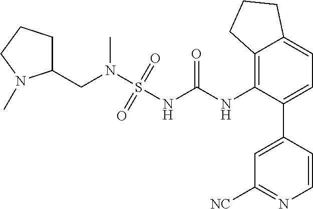 [SMILES: CN1CCCC1CN(C)S(=O)(=O)NC(=O)Nc1c(-c2ccnc(C#N)c2)ccc2c1CCC2] | ¹H NMR (Methanol-d₄) δ 8.68 (dd, 1H), 8.03 (dd, 1H), 7.78 (dd, 1H), 7.23 (d, 2H), 3.72 (dd, 1H), 3.51 (d, 1H), 3.19-3.09 (m, 1H), 3.09-2.91 (m, 6H), 2.79 (s, 3H), 2.71 (s, 3H), 2.13 (p, 2H), 1.99 (m, 3H), 1.65 (m, 1H). | m/z 469.4 (M + H)⁺ (ES⁺) | 468.58 |
| | 3-(N-Methyl-N-((1-methylpyrrolidin-2- | | | |
| | yl)methyl)sulfamoyl)-1-(5-(2- | | | |
| | cyanopyridin-4-yl)-2,3-dihydro-1H-inden- | | | |
| | 4-yl)urea | | | |
| 322 | 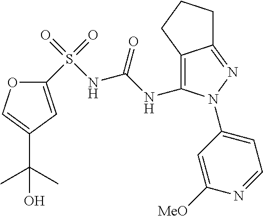 [SMILES: COc1cc(-n2nc3c(c2NC(=O)NS(=O)(=O)c2cc(C(C)(C)O)co2)CCC3)ccn1] | 1H NMR (DMSO-d6) δ 8.09 (d, J =6.o Hz, 1H), 7.39 (s, 1H), 7.25 (d, J = 6.0 Hz, 1H), 7.02 (s, 1H), 6.58 (s, 1H), 3.86 (s, 3H), 2.63-2.58 (m, 2H), 2.58- 2.54 (m, 2H), 2.29-2.25 (m, 2H), 1.35 (s, 6H), NH and OH missing. | m/z 462.0 (M + H)⁺ (ES⁺) | 461.49 |
| | 4-(2-Hydroxypropan-2-yl)-N-((2-(2- | | | |
| | methoxypyridin-4-yl)-2,4,5,6- | | | |
| | tetrahydrocyclopenta[c]pyrazol-3- | | | |
| | yl)carbamoyl)furan-2-sulfonamide | | | |
| 323 | 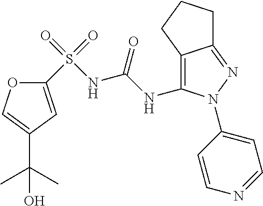 [SMILES: CC(C)(O)c1coc(S(=O)(=O)NC(=O)Nc2c3c(nn2-c2ccncc2)CCC3)c1] | 1H NMR (DMSO- d6 + D2O) δ8.52 (d, J = 6.0 Hz, 2H), 7.60 (d, J = 6.0 Hz, 2H), 7.50 (s, 1H), 6.73 (s, 1H), 2.66-2.61 (m, 2H), 2.56-2.52 (m, 2H), 2.32- 2.25 (m, 2H), 1.36 (s, 6H), NH & OH missing. | m/z 432.0 (M + H)⁺ (ES⁺) | 431.47 |
| | 4-(2-Hydroxypropan-2-yl)-N-((2- | | | |
| | (pyridin-4-yl)-2,4,5,6- | | | |
| | tetrahydrocyclopenta[c]pyrazol-3- | | | |
| | yl)carbamoyl)furan-2-sulfonamide | | | |

### EXAMPLES—BIOLOGICAL STUDIES

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
1. Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
2. Add 5 μcompound (8 points half-log dilution, with 10 μM top dose) or vehicle (DMSO 0.1% FAC) to the appropriate wells
3. Incubate for 3 hrs at 37° C. and 5% CO₂
4. Add 5 μnigericin (Sigma #N7143) (FAC 5 μM) to all wells
5. Incubate for 1 hr at 37° C. and 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

96-well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Cornp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Camp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

High MCC950 (10 uM)
LOW Drug free control
Compound 8-point half-log dilution

The results of the pyroptosis assay performed are summarised in Table 3 below as THP IC₅₀.

### Human Whole Blood IL1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Plate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 3 below as HWB IC₅₀.

TABLE 3
NLRP3 inhibitory activity (≤0.5 μM = ‘++++’, ≤1 μM = ‘+++’,
≤5 μM = ‘++’, ≤10 μM = ‘+’, not determined = ‘ND’).

| Example No | THP IC₅₀ | HWB IC₅₀ |
|---|---|---|
| 1 | ++++ | ++++ |
| 2 | ++ | ND |
| 3 | ++ | ND |
| 4 | ++ | ND |
| 5 | ++ | ND |
| 6 | ++ | ND |
| 7 | ++ | ND |
| 8 | ++ | ND |
| 9 | ++ | ND |
| 10 | ++++ | ++++ |
| 11 | ++++ | ND |
| 12 | ++++ | +++ |
| 13 | ++++ | ND |
| 14 | ++ | ND |
| 15 | ++ | ND |
| 16 | ++ | ND |
| 17 | ++ | ND |
| 18 | +++ | ND |
| 19 | +++ | ND |
| 20 | ++ | ND |
| 21 | ++ | ND |
| 22 | +++ | ND |
| 23 | ++++ | +++ |
| 24 | ++++ | +++ |
| 25 | ++++ | ++++ |
| 26 | ++ | ND |
| 27 | ++++ | ++ |
| 28 | ++++ | ++++ |
| 29 | ++ | ND |
| 30 | ++ | ++++ |
| 31 | ++ | ND |
| 32 | ++++ | ++++ |
| 33 | ++++ | ++++ |
| 34 | +++ | ++++ |
| 35 | ++++ | +++ |
| 36 | ++++ | ++++ |
| 37 | ++++ | ++++ |
| 38 | ++++ | ++++ |
| 39 | +++ | +++ |
| 40 | ++++ | +++ |
| 41 | ++++ | +++ |
| 42 | ++ | ND |
| 43 | ++ | ND |
| 44 | ++++ | ++++ |
| 45 | ++++ | ++++ |
| 46 | +++ | ++ |
| 47 | +++ | ND |
| 48 | ++++ | ++++ |
| 49 | ++++ | ++++ |
| 50 | ++ | ND |
| 51 | ++++ | ++++ |
| 52 | ++++ | ++++ |
| 53 | ++++ | ++ |
| 54 | ++++ | ++++ |
| 55 | ++++ | +++ |
| 56 | ++++ | ++++ |
| 57 | +++ | +++ |
| 58 | ++++ | ++++ |
| 59 | ++++ | +++ |
| 60 | ++++ | ++++ |
| 61 | ++++ | ++++ |
| 62 | ++++ | ++ |
| 63 | ++++ | ++++ |
| 64 | ++++ | ++++ |
| 65 | ++++ | ++++ |
| 66 | ++++ | ++++ |
| 67 | ++ | ND |
| 68 | ++++ | +++ |
| 69 | ++++ | ++++ |
| 70 | ++++ | ++++ |
| 71 | ++++ | +++ |
| 72 | ++++ | +++ |
| 73 | ++++ | ++++ |
| 74 | + | ND |
| 75 | ++++ | ND |
| 76 | ++++ | ++++ |
| 77 | ++++ | +++ |
| 78 | + | ND |
| 79 | ++++ | +++ |
| 80 | ++++ | +++ |
| 81 | ++++ | +++ |
| 82 | + | ND |
| 83 | ++ | ND |
| 84 | +++ | +++ |
| 85 | +++ | ND |
| 86 | + | ND |
| 87 | ++ | ++ |
| 88 | +++ | ND |
| 89 | ++++ | +++ |
| 90 | ++++ | +++ |
| 91 | ++++ | +++ |
| 92 | +++ | ND |
| 93 | + | ND |
| 94 | ++ | ND |
| 95 | ++++ | +++ |
| 96 | +++ | ND |
| 97 | ++ | ND |
| 98 | ++ | ND |
| 99 | + | ND |
| 100 | ++ | ++ |
| 101 | ++ | +++ |
| 102 | ++++ | ND |
| 103 | ++++ | ND |
| 104 | ++++ | ND |
| 105 | ++++ | +++ |
| 106 | ++++ | ++ |
| 107 | ++++ | ND |
| 108 | ++++ | ++ |
| 109 | ++++ | ++ |
| 110 | + | ND |
| 111 | ++ | ND |
| 112 | +++ | ND |
| 113 | ++ | ND |
| 114 | ++++ | ++ |
| 115 | ++++ | ND |
| 116 | ++++ | ++++ |
| 117 | ++++ | +++ |
| 118 | ++++ | +++ |
| 119 | ++++ | ++ |
| 120 | ++++ | ++ |
| 121 | ++++ | +++ |
| 122 | ++++ | ++++ |
| 123 | ++++ | ++++ |
| 124 | ++++ | ++++ |
| 125 | ++++ | ++++ |
| 126 | ++++ | ++++ |
| 127 | + | ND |
| 128 | +++ | ND |
| 129 | ++ | ND |
| 130 | ++ | ND |
| 131 | + | ND |
| 132 | +++ | ND |
| 133 | ++++ | ND |
| 134 | +++ | ND |
| 135 | +++ | ND |
| 136 | ++++ | ND |
| 137 | ++++ | ND |
| 138 | +++ | + |
| 139 | ++ | ND |
| 140 | ++ | ND |
| 141 | +++ | ND |
| 142 | ++ | ND |
| 143 | +++ | ND |
| 144 | ++ | ND |
| 145 | ++++ | +++ |
| 146 | +++ | ND |
| 147 | ++++ | ND |
| 148 | ++++ | ND |
| 149 | ++++ | ++++ |
| 150 | ++++ | ND |
| 151 | ++ | ND |
| 152 | ++++ | ND |
| 153 | ++++ | ++ |
| 154 | ++ | ND |
| 155 | ++++ | ND |
| 156 | ++++ | ND |
| 157 | ++ | ND |
| 158 | + | ND |
| 159 | + | ND |
| 160 | + | ND |
| 161 | + | ND |
| 162 | + | ND |
| 163 | ++++ | ++++ |
| 164 | ++++ | ++++ |
| 165 | ++ | ND |
| 166 | ++ | + |
| 167 | ++++ | ++++ |
| 168 | ++++ | ++ |
| 169 | ++++ | ++++ |
| 170 | ++++ | +++ |
| 171 | ++++ | ++ |
| 172 | ++++ | +++ |
| 173 | ++++ | ++++ |
| 174 | ++++ | +++ |
| 175 | ++++ | ++++ |
| 176 | ++++ | ++++ |
| 177 | ++++ | +++ |
| 178 | ++++ | ++ |
| 179 | ++ | ++ |
| 180 | ++++ | ++++ |
| 181 | ++++ | ++++ |
| 182 | ++++ | ++++ |
| 183 | ++++ | ++++ |
| 184 | ++++ | ++++ |
| 185 | ++++ | ++++ |
| 186 | ++++ | ++++ |
| 187 | ++++ | ++++ |
| 188 | ++++ | ++++ |
| 189 | ++++ | ++++ |
| 190 | ++++ | ++++ |
| 191 | ++++ | +++ |
| 192 | ++++ | ++++ |
| 193 | ++++ | ++++ |
| 194 | +++ | ND |
| 195 | ++++ | ++++ |
| 196 | ++++ | ++++ |
| 197 | ++++ | ++++ |
| 198 | ++++ | ++++ |
| 199 | ++++ | ++++ |
| 200 | ++++ | ++++ |
| 201 | ++++ | ++++ |
| 202 | ++++ | ++++ |
| 203 | ++++ | ++++ |
| 204 | ++ | ND |
| 205 | ++++ | ++++ |
| 206 | ++++ | ++ |
| 207 | +++ | ++ |
| 208 | ++ | ND |
| 209 | ++++ | ++ |
| 210 | ++++ | ++ |
| 211 | ++++ | ++ |
| 212 | ++ | ND |
| 213 | ++++ | ++ |
| 214 | ++++ | +++ |
| 215 | ++++ | ++++ |
| 216 | ++++ | ++ |
| 217 | + | ND |
| 218 | ++++ | +++ |
| 219 | ++++ | ++++ |
| 220 | + | ND |
| 221 | ++++ | ++++ |
| 222 | ++++ | ++++ |
| 223 | ++++ | ++++ |
| 224 | ++++ | ++++ |
| 225 | ++++ | ++ |
| 226 | ++++ | +++ |
| 227 | ++++ | ++++ |
| 228 | ++++ | ++++ |
| 229 | ++++ | ++++ |
| 230 | ++++ | ++++ |
| 231 | ++++ | ++++ |
| 232 | ++++ | +++ |
| 233 | ++++ | +++ |
| 234 | ++++ | +++ |
| 235 | ++++ | ++++ |
| 236 | ++++ | ++++ |
| 237 | ++++ | +++ |
| 238 | ++ | ND |
| 239 | ++++ | ++++ |
| 240 | ++ | ND |
| 241 | ++ | ND |
| 242 | ++++ | ++++ |
| 243 | ++++ | ++++ |
| 244 | ++++ | ++++ |
| 245 | ++++ | ++++ |
| 246 | ++++ | +++ |
| 247 | ++++ | ++++ |
| 248 | ++++ | ++++ |
| 249 | ++++ | ++++ |
| 250 | ++++ | ++++ |
| 251 | ++++ | ++++ |
| 252 | ++++ | ++++ |
| 253 | +++ | ++ |
| 254 | ++ | ND |
| 255 | ++++ | ++++ |
| 256 | +++ | ND |
| 257 | ++++ | ++++ |
| 258 | ++++ | ++++ |
| 259 | ++++ | ++++ |
| 260 | ++++ | ++ |
| 261 | ++++ | ++ |
| 262 | ++++ | +++ |
| 263 | ++++ | ++++ |
| 264 | ++++ | ++++ |
| 265 | ++++ | ++++ |
| 266 | ++++ | +++ |
| 267 | ++++ | ++++ |
| 268 | ++ | ND |
| 269 | ++++ | ++++ |
| 270 | ++++ | ++++ |
| 271 | ++++ | ++++ |
| 272 | ++ | ND |
| 273 | ++ | ND |
| 274 | ++++ | ++++ |
| 275 | ++++ | ++++ |
| 276 | ++++ | +++ |
| 277 | ++++ | ++++ |
| 278 | ++++ | ++++ |
| 279 | ++++ | ++++ |
| 280 | ++++ | ++++ |
| 281 | ++++ | ++++ |
| 282 | ++++ | ++++ |
| 283 | ++ | ND |
| 284 | ++++ | +++ |
| 285 | ++++ | ++ |
| 286 | ++++ | + |
| 287 | ++++ | ++++ |
| 288 | ++++ | ++ |
| 289 | ++++ | ++ |
| 290 | ++++ | ++++ |
| 291 | ++++ | +++ |
| 292 | ++++ | +++ |
| 293 | ++++ | ++ |
| 294 | ++++ | ++ |
| 295 | ++++ | +++ |
| 296 | ++++ | ++++ |
| 297 | +++ | ND |
| 298 | ++++ | +++ |
| 299 | ++++ | ++++ |
| 300 | ++++ | +++ |
| 301 | +++ | ND |
| 302 | ++ | ND |
| 303 | ++++ | ++++ |
| 304 | ++++ | ++++ |
| 305 | ++++ | ++++ |
| 306 | ++++ | ++++ |
| 307 | ++++ | ++++ |
| 308 | ++++ | +++ |
| 309 | ++ | ND |
| 310 | ++++ | ++++ |
| 311 | ++++ | ++++ |
| 312 | ++++ | ++++ |
| 313 | ++++ | ++++ |
| 314 | ++++ | +++ |
| 315 | ++ | ND |
| 316 | ++ | ND |
| 317 | ++++ | ++++ |
| 318 | +++ | ++++ |
| 319 | + | ND |
| 320 | ++++ | ++++ |
| 321 | +++ | ++++ |
| 322 | ++ | ND |
| 323 | ++ | ++++ |

### PK Protocol

Pharmacokinetic parameters were determined in male Sprague Dawley rats (Charles River, UK, 250-350 g; or Vital River Laboratory Animal Technology Co Ltd, Beijing, China, 7-9 weeks old). Animals were individually housed during the study and maintained under a 12 h light/dark cycle. Animals had free access to food and water.

For intravenous administration, compounds were formulated as a solution in water or DMSO:PBS [10:90] in 2 mL/kg dosing volume and administered via tail vein. For oral administration, compounds were formulated as a solution in DMSO:water [10:90] in 5 mL/kg dosing volume and administered orally.

Serial blood samples (about 120-300 μL) were taken from each animal at each of 8 time-points post dose (0.083, 0.25, 0.5, 1, 2, 4, 8 and 24 h) or at each of 12 time-points post dose (0.03, 0.1, 0.17, 0.25, 0.5, 1, 2, 4, 6, 8, 12 and 24 h) or pre-dose and at each of 9 time-points post dose (0.25, 0.5, 1, 2, 4, 6, 8, 12 and 24 h). Samples were held on ice for no longer than 30 minutes before centrifugation (10,000 rpm (8,385 g) for 3 minutes; or 5,696 rpm (3,000 g) for 15 minutes) for plasma generation. Plasma was frozen on dry ice prior to bioanalysis. PK parameters were generated from LC-MS/MS data using Dotmatics or Phoenix WinNonlin 6.3 software.

TABLE 4
PK data (intravenous administration)

| Example | Dose | AUC | T_{1/2} | V_{dss} | Cl |
|---|---|---|---|---|---|
| No | (mg/kg) | (ng · hr/mL) | (hr) | (L/kg) | (mL/min/kg) |
| 1 | 1 | 340.9 | 3.3 | 4.49 | 49.1 |
| 10 | 1.98 | 806.3 | 3.8 | 7.14 | 40.9 |
| 25 | 1 | 411.5 | 0.4 | 0.41 | 41.9 |
| 28 | 1 | 385.5 | 1.2 | 1.07 | 43.3 |
| 30 | 1 | 182.5 | 2.3 | 2.72 | 91.3 |
| 33 | 1 | 274.4 | 6.3 | 3.95 | 60.7 |
| 34 | 1 | 661.2 | 12.1 | 3.03 | 25.2 |
| 36 | 1 | 356.0 | 0.5 | 0.8 | 47.6 |
| 37 | 1 | 247.8 | 0.3 | 1.32 | 67.2 |
| 38 | 1 | 375.4 | 1.2 | 1.14 | 51.5 |
| 44 | 1 | 259.0 | 0.5 | 0.75 | 64.9 |
| 45 | 1 | 259.1 | 0.5 | 0.98 | 65.6 |
| 51 | 1 | 600.1 | 3.7 | 3.68 | 27.8 |
| 54 | 1 | 200 | 1.6 | 2.5 | 85.4 |
| 55 | 1 | 1862.5 | 6.9 | 3.25 | 9.8 |
| 58 | 1 | 591.0 | 0.9 | 0.55 | 28.2 |
| 60 | 1 | 300.2 | 1.0 | 1.22 | 56.3 |
| 61 | 1.69 | 1145.0 | 1.2 | 0.6 | 24.6 |
| 64 | 1.7 | 2710.4 | 3.8 | 1.23 | 10.5 |
| 69 | 1 | 896 | 5.8 | 0.78 | 19 |
| 70 | 1 | 510.7 | 1.1 | 1.21 | 32.6 |
| 73 | 1 | 1518.0 | 1.0 | 0.31 | 11.0 |
| 103 | 1 | 346.3 | 2.2 | 1.77 | 48.1 |
| 104 | 1 | 841.0 | 1.2 | 1.04 | 19.8 |
| 122 | 1 | 1318.7 | 11.1 | 8.69 | 12.6 |
| 205 | 1 | 4872.7 | 2.6 | 0.57 | 3.4 |
| 221 | 1 | 65385 | 15 | 0.36 | 0.39 |
| 223 | 1 | 1741.8 | 0.9 | 0.31 | 9.6 |
| 224 | 1 | 6480.1 | 3.2 | 0.47 | 2.6 |
| 230 | 1 | 1949.8 | 1.3 | 0.24 | 8.5 |
| 251 | 1 | 1257.3 | 2.9 | 2.12 | 13.3 |

TABLE 5
PK data (oral administration)

| Example | Dose | Cₘₐₓ | AUC | Tₘₐₓ | T_{1/2} | Cl/F | |
|---|---|---|---|---|---|---|---|
| No | (mg/kg) | (ng/mL) | (ng · hr/mL) | (hr) | (hr) | (mL/min/kg) | Bioavailability |
| 69 | 3 | 113 | 504 | 0.5 | 5.0 | 112 | 19 |
| 221 | 3 | 7220 | 135743 | 0.75 | 12 | 0.48 | 83 |

As is evident from the results presented in Table 3, surprisingly in spite of the structural differences versus the prior art compounds, the compounds of the invention show high levels of NLRP3 inhibitory activity in the pyroptosis assay and in particular in the human whole blood assay.

As is evident from the results presented in Tables 4 and 5, the compounds of the invention show advantageous pharmacokinetic properties, for example half-life T_{1/2}, area under the curve AUC, clearance Cl and/or bioavailability, compared to the prior art compounds. In particular, it is evident from the pharmacokinetic data that the compounds of the invention are particularly suited to topical routes of administration.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 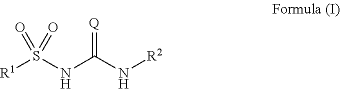
or a pharmaceutically acceptable salt, solvate or prodrug thereof, wherein:
Q is selected from O or S;
R¹ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R² is a cyclic group substituted at the α-position with a monovalent heterocyclic group or a monovalent aromatic group, wherein a ring atom of the heterocyclic or aromatic group is directly attached to a ring atom of the cyclic group, wherein the heterocyclic or aromatic group may optionally be substituted, and wherein the cyclic group may optionally be further substituted.

2. The compound or a pharmaceutically acceptable salt, solvate or prodrug thereof as claimed in claim 1, wherein R¹ is a 4- to 10-membered cyclic group, wherein the cyclic group may optionally be substituted.

3. The compound or a pharmaceutically acceptable salt, solvate or prodrug thereof as claimed in claim 1, wherein R¹ is a C₁-C₁₅ alkyl, C₂-C₁₅ alkenyl or C₂-C₁₅ alkynyl group, all of which may optionally be substituted, and all of which may optionally include one, two or three heteroatoms N, O or S in their carbon skeleton.

4. The compound or a pharmaceutically acceptable salt, solvate or prodrug thereof as claimed in claim 1, wherein R¹ is substituted with one, two or three substituents independently selected from halo; —CN; —N₃; —R^{β}; —OH; —OR^{β}; —SO₂R^{β}; —NH₂; —NHR^{β}; —N(R^{β})₂; —R^{α}—NH₂; —R^{α}—NHR^{β}; —R^{α}—N(R^{β})₂; —COR^{β}; —COOR^{β}; —OCOR^{β}; —R^{α}—COR^{β}; —R^{α}—COOR^{β}; —R^{α}—OCOR^{β}; —CONH₂; —CONHR^{β}; —CON(R^{β})₂; or oxo (═O);
wherein each —R^{α}— is independently selected from a C₁-C₆ alkylene group, wherein one or two carbon atoms in the backbone of the alkylene group may optionally be replaced by one or two heteroatoms N, O or S, and wherein the alkylene group may optionally be substituted with one or two halo and/or —R^{β} groups; and
wherein each —R^{β} is independently selected from a C₁-C₆ alkyl, C₂-C₆ alkenyl, C₂-C₆ alkynyl or C₂-C₆ cyclic group, and wherein any —R^{β} may optionally be substituted with one, two or three C₁-C₄ alkyl, C₁-C₄ haloalkyl, C₃-C₇ cycloalkyl, —O(C₁-C₄ alkyl), —O(C₁-C₄ haloalkyl), —O(C₃-C₇ cycloalkyl), halo, —OH, —NH₂, —CN, —C≡CH or oxo (═O) groups.

5. The compound or a pharmaceutically acceptable salt, solvate or prodrug thereof as claimed in claim 1, wherein the α-substituted cyclic group of R² is a 5- or 6-membered cyclic group, wherein the cyclic group may optionally be further substituted.

6. The compound or a pharmaceutically acceptable salt, solvate or prodrug thereof as claimed in claim 1, wherein the monovalent heterocyclic or aromatic group at the α-position of the cyclic group of R² is phenyl or a 5- or 6-membered heterocyclic group, all of which may optionally be substituted.

7. The compound or a pharmaceutically acceptable salt, solvate or prodrug thereof as claimed in claim 1, wherein the monovalent heterocyclic or aromatic group at the α-position of the cyclic group of R² is phenyl, pyridinyl, pyrimidinyl or pyrazolyl, all of which may optionally be substituted with one or two substituents independently selected from halo, —OH, —NH₂, —CN, C₁-C₃ alkyl or —O(C₁-C₃ alkyl) groups.

8. The compound or a pharmaceutically acceptable salt, solvate or prodrug thereof as claimed in claim 1, wherein the cyclic group of R² is further substituted with one or two substituents independently selected from halo, —R^{δ}, —OR^{δ} or —COR^{δ} groups, wherein each R^{δ} is independently selected from a C₁-C₆ alkyl, C₂-C₆ alkenyl, C₂-C₆ alkynyl or C₂-C₆ cyclic group, and wherein each R^{δ} is optionally further substituted with one or more halo groups.

9. The compound or a pharmaceutically acceptable salt, solvate or prodrug thereof as claimed in claim 1, wherein Q is O.

10. The compound or a pharmaceutically acceptable salt, solvate or prodrug thereof as claimed in claim 1, wherein the compound is selected from the group consisting of:

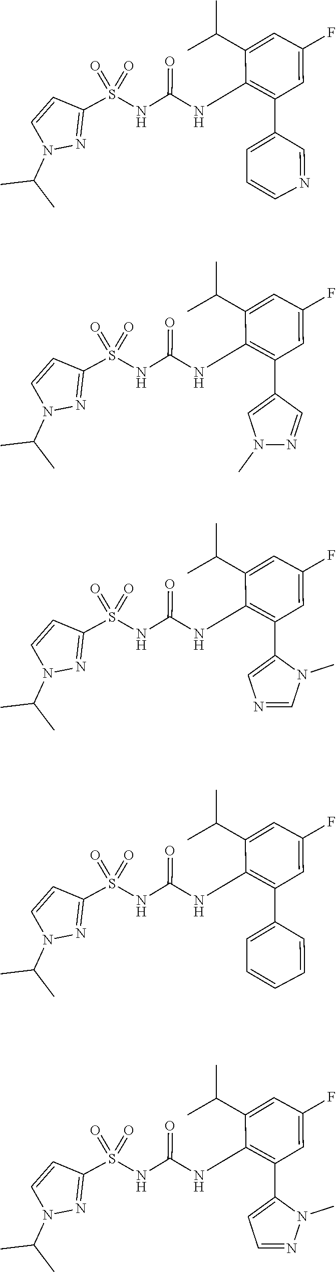

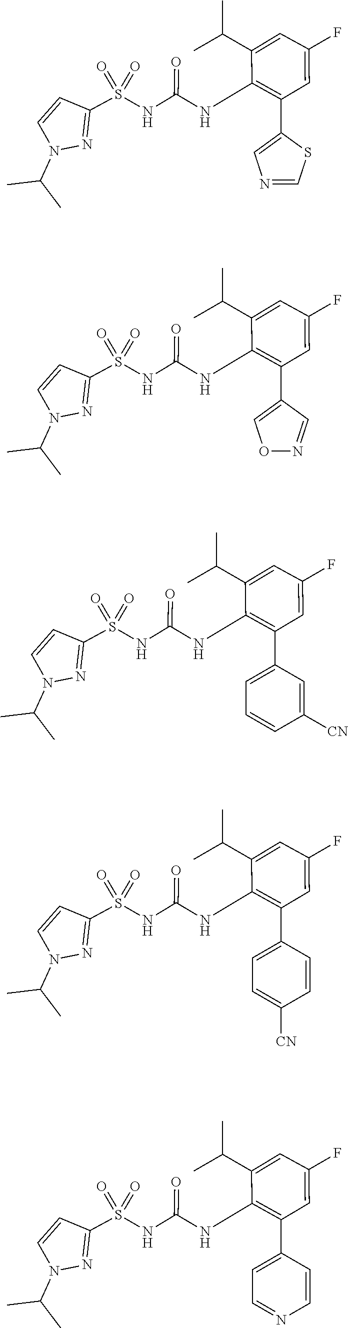 [SMILES: CC(C)c1cc(F)cc(-c2cncs2)c1NC(=O)NS(=O)(=O)c1ccn(C(C)C)n1 ; CC(C)c1cc(F)cc(-c2cnoc2)c1NC(=O)NS(=O)(=O)c1ccn(C(C)C)n1 ; CC(C)c1cc(F)cc(-c2cccc(C#N)c2)c1NC(=O)NS(=O)(=O)c1ccn(C(C)C)n1 ; CC(C)c1cc(F)cc(-c2ccc(C#N)cc2)c1NC(=O)NS(=O)(=O)c1ccn(C(C)C)n1 ; CC(C)c1cc(F)cc(-c2ccncc2)c1NC(=O)NS(=O)(=O)c1ccn(C(C)C)n1]

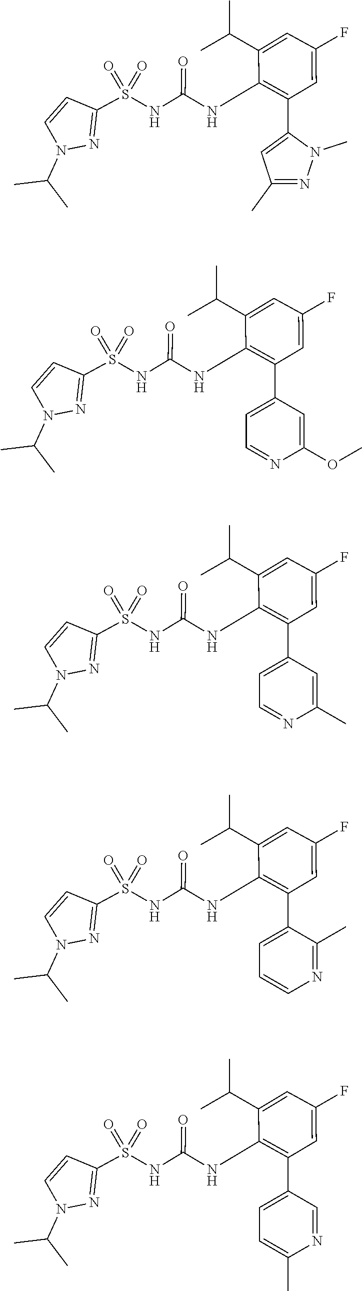

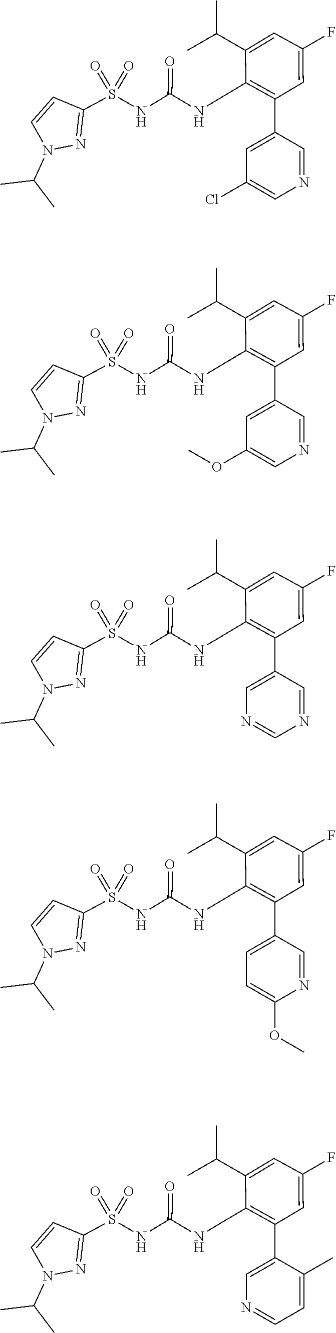 [SMILES: CC(C)c1cc(F)cc(-c2cncc(Cl)c2)c1NC(=O)NS(=O)(=O)c1ccn(C(C)C)n1 ; COc1cncc(-c2cc(F)cc(C(C)C)c2NC(=O)NS(=O)(=O)c2ccn(C(C)C)n2)c1 ; CC(C)c1cc(F)cc(-c2cncnc2)c1NC(=O)NS(=O)(=O)c1ccn(C(C)C)n1 ; COc1ccc(-c2cc(F)cc(C(C)C)c2NC(=O)NS(=O)(=O)c2ccn(C(C)C)n2)cn1 ; Cc1ccncc1-c1cc(F)cc(C(C)C)c1NC(=O)NS(=O)(=O)c1ccn(C(C)C)n1]

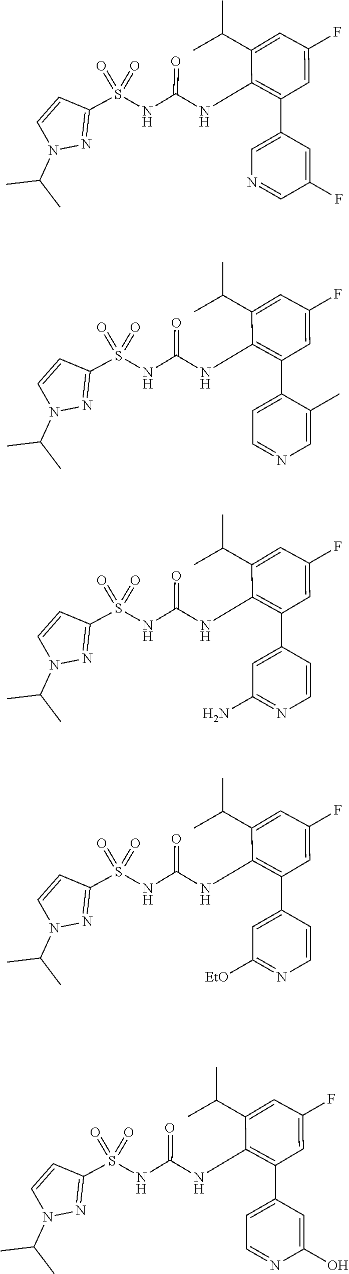

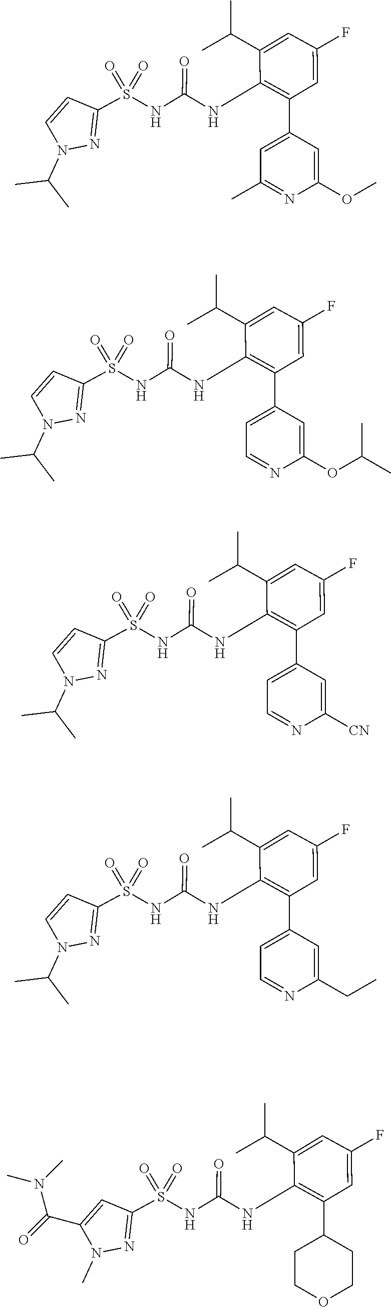

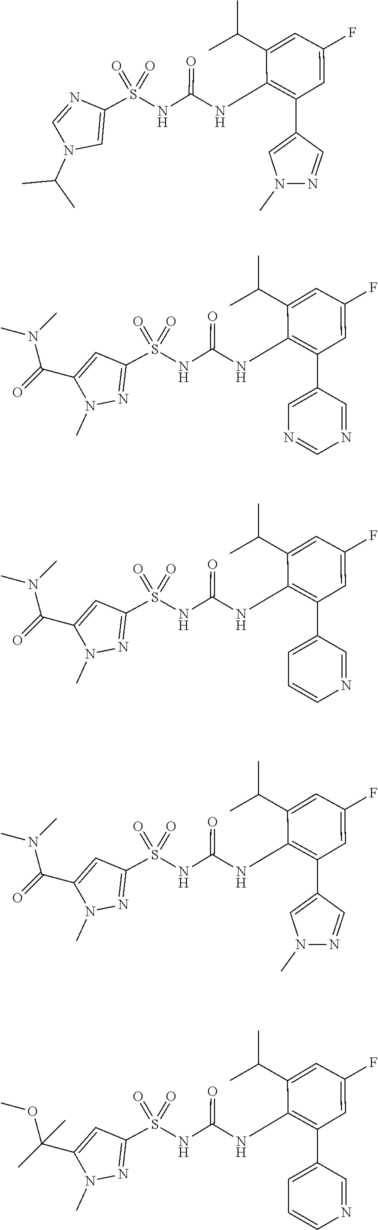

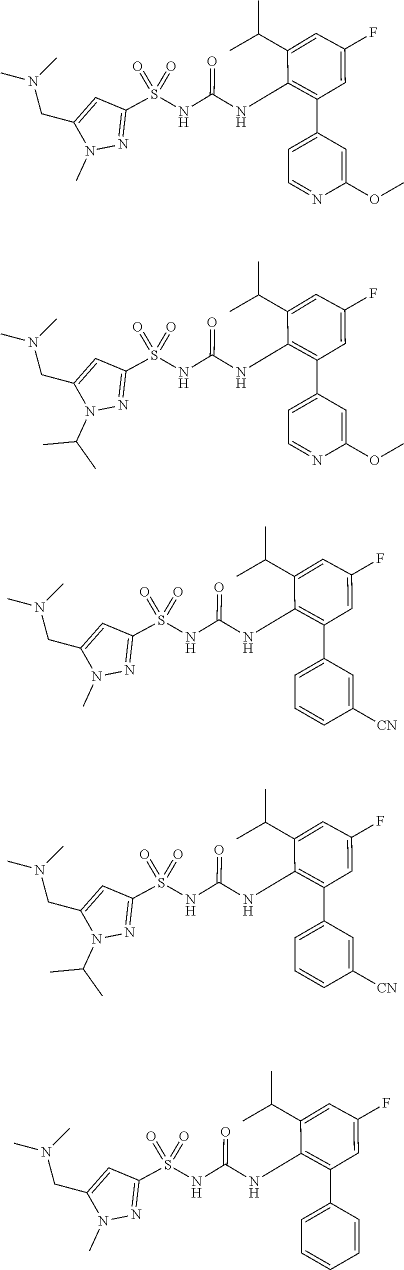

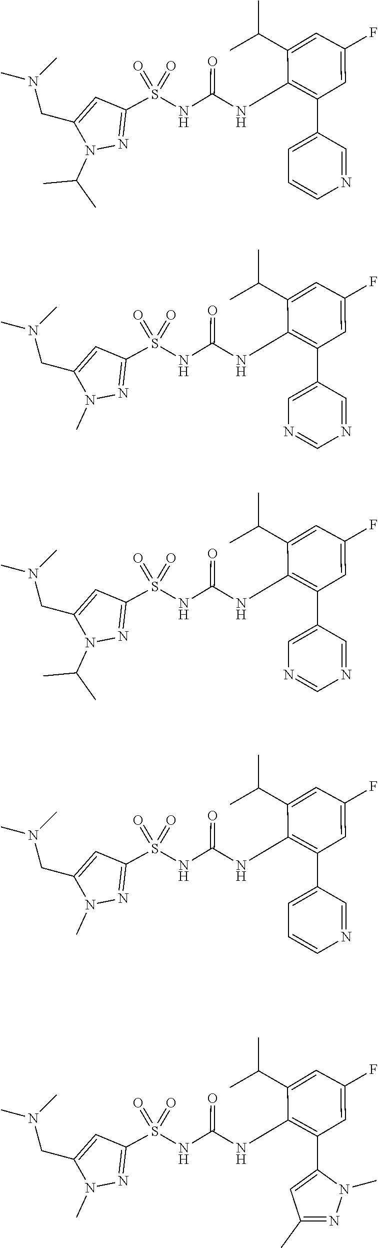

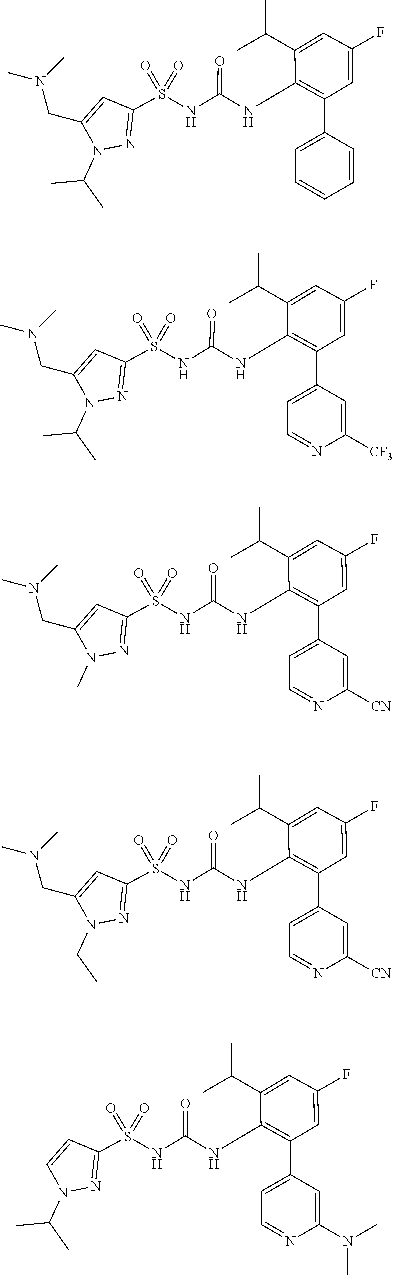 [SMILES: CC(C)c1cc(F)cc(-c2ccccc2)c1NC(=O)NS(=O)(=O)c1cc(CN(C)C)n(C(C)C)n1 ; CC(C)c1cc(F)cc(-c2ccnc(C(F)(F)F)c2)c1NC(=O)NS(=O)(=O)c1cc(CN(C)C)n(C(C)C)n1 ; CC(C)c1cc(F)cc(-c2ccnc(C#N)c2)c1NC(=O)NS(=O)(=O)c1cc(CN(C)C)n(C)n1 ; CCn1nc(S(=O)(=O)NC(=O)Nc2c(-c3ccnc(C#N)c3)cc(F)cc2C(C)C)cc1CN(C)C ; CC(C)c1cc(F)cc(-c2ccnc(N(C)C)c2)c1NC(=O)NS(=O)(=O)c1ccn(C(C)C)n1]

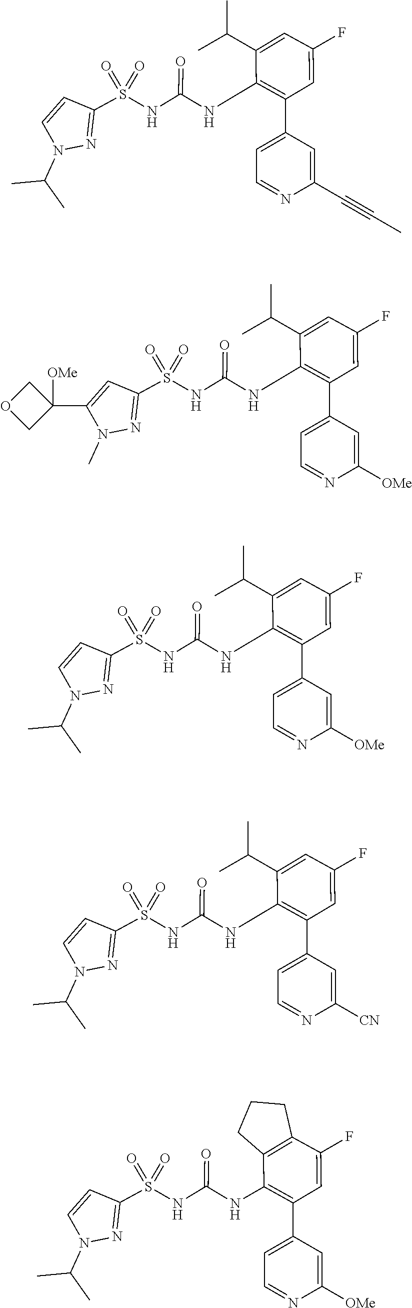

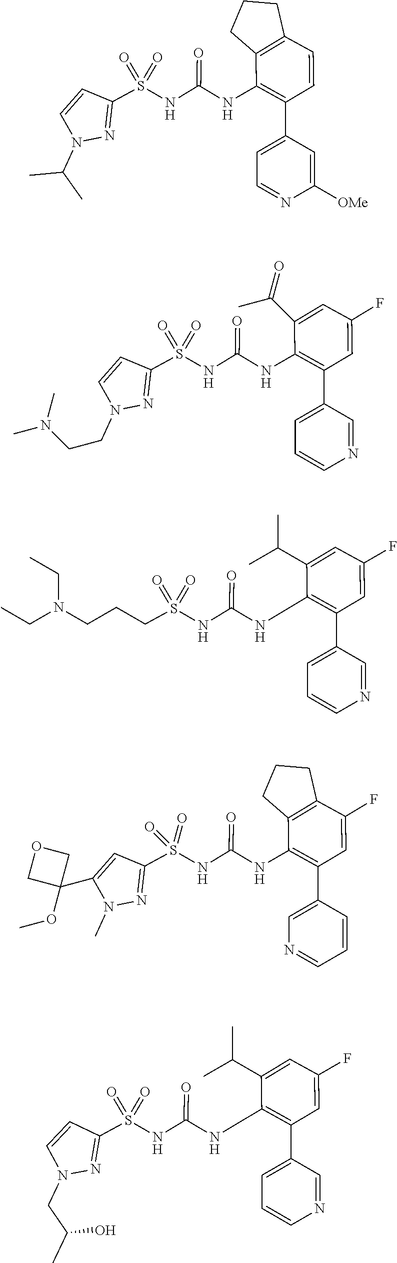 [SMILES: COc1cc(-c2ccc3c(c2NC(=O)NS(=O)(=O)c2ccn(C(C)C)n2)CCC3)ccn1 ; CC(=O)c1cc(F)cc(-c2cccnc2)c1NC(=O)NS(=O)(=O)c1ccn(CCN(C)C)n1 ; CCN(CC)CCCS(=O)(=O)NC(=O)Nc1c(-c2cccnc2)cc(F)cc1C(C)C ; COC1(c2cc(S(=O)(=O)NC(=O)Nc3c(-c4cccnc4)cc(F)c4c3CCC4)nn2C)COC1 ; CC(C)c1cc(F)cc(-c2cccnc2)c1NC(=O)NS(=O)(=O)c1ccn(C[C@@H](C)O)n1]

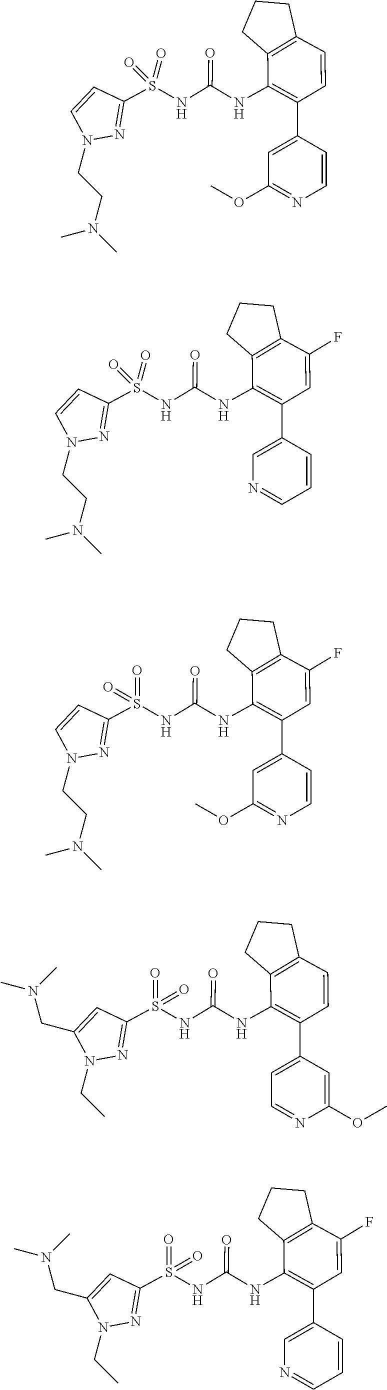

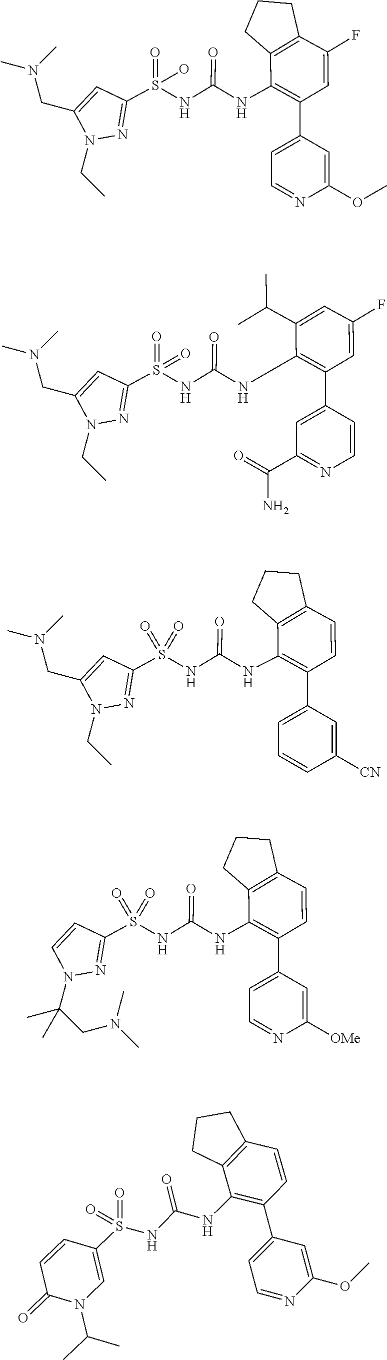

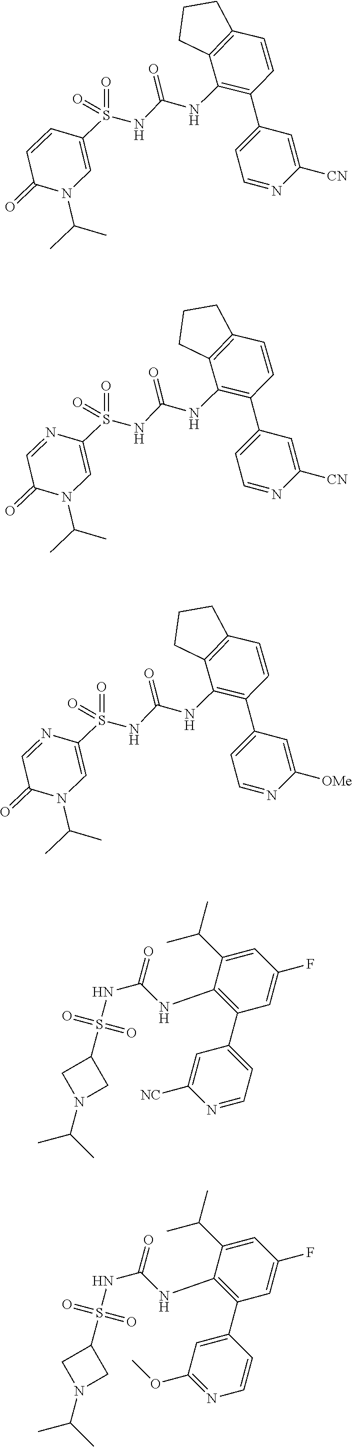 [SMILES: CC(C)n1cc(S(=O)(=O)NC(=O)Nc2c(-c3ccnc(C#N)c3)ccc3c2CCC3)ccc1=O ; CC(C)n1cc(S(=O)(=O)NC(=O)Nc2c(-c3ccnc(C#N)c3)ccc3c2CCC3)ncc1=O ; COc1cc(-c2ccc3c(c2NC(=O)NS(=O)(=O)c2cn(C(C)C)c(=O)cn2)CCC3)ccn1 ; CC(C)c1cc(F)cc(-c2ccnc(C#N)c2)c1NC(=O)NS(=O)(=O)C1CN(C(C)C)C1 ; COc1cc(-c2cc(F)cc(C(C)C)c2NC(=O)NS(=O)(=O)C2CN(C(C)C)C2)ccn1]

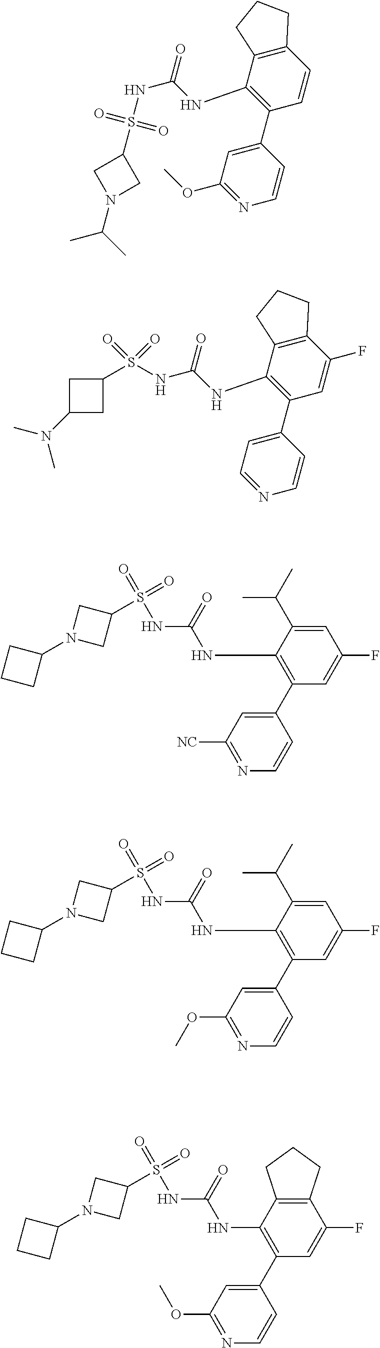 [SMILES: COc1cc(-c2ccc3c(c2NC(=O)NS(=O)(=O)C2CN(C(C)C)C2)CCC3)ccn1 ; CN(C)C1CC(S(=O)(=O)NC(=O)Nc2c(-c3ccncc3)cc(F)c3c2CCC3)C1 ; CC(C)c1cc(F)cc(-c2ccnc(C#N)c2)c1NC(=O)NS(=O)(=O)C1CN(C2CCC2)C1 ; COc1cc(-c2cc(F)cc(C(C)C)c2NC(=O)NS(=O)(=O)C2CN(C3CCC3)C2)ccn1 ; COc1cc(-c2cc(F)c3c(c2NC(=O)NS(=O)(=O)C2CN(C4CCC4)C2)CCC3)ccn1]

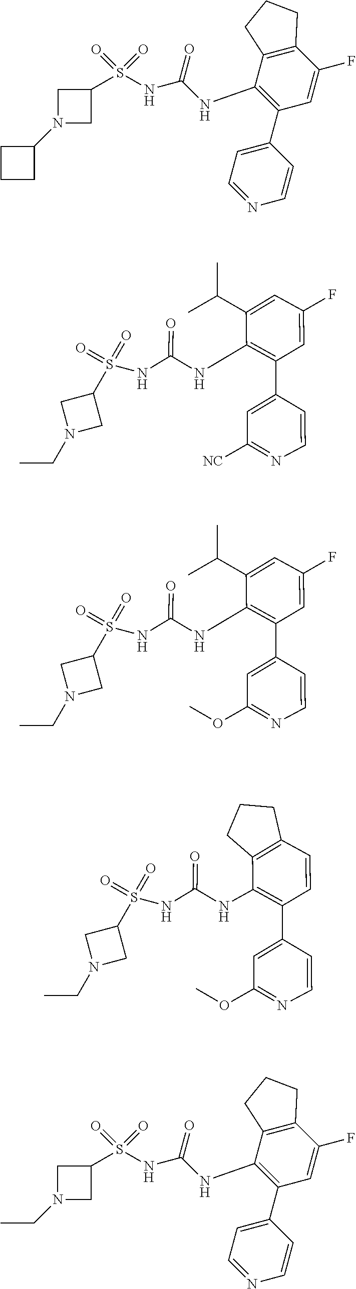 [SMILES: O=C(Nc1c(-c2ccncc2)cc(F)c2c1CCC2)NS(=O)(=O)C1CN(C2CCC2)C1 ; CCN1CC(S(=O)(=O)NC(=O)Nc2c(-c3ccnc(C#N)c3)cc(F)cc2C(C)C)C1 ; CCN1CC(S(=O)(=O)NC(=O)Nc2c(-c3ccnc(OC)c3)cc(F)cc2C(C)C)C1 ; CCN1CC(S(=O)(=O)NC(=O)Nc2c(-c3ccnc(OC)c3)ccc3c2CCC3)C1 ; CCN1CC(S(=O)(=O)NC(=O)Nc2c(-c3ccncc3)cc(F)c3c2CCC3)C1]

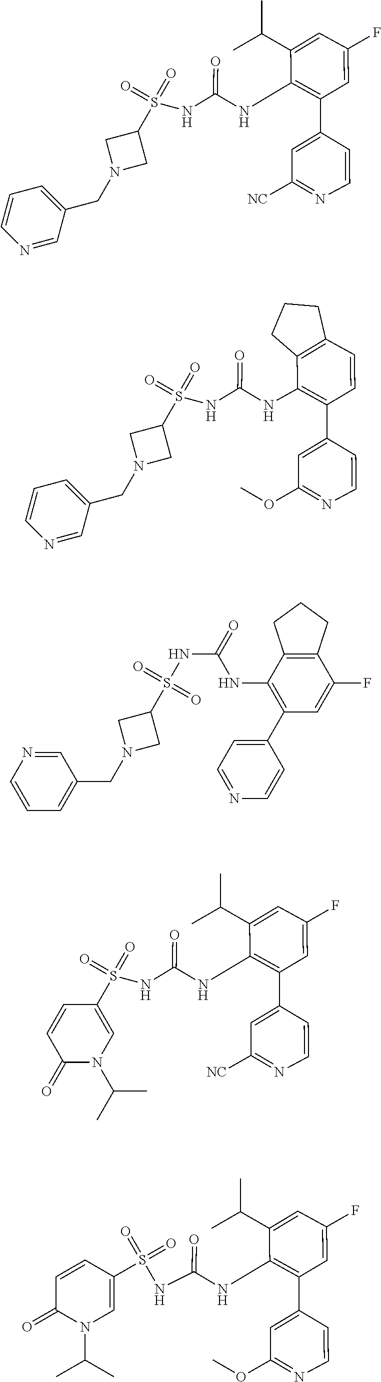 [SMILES: CC(C)c1cc(F)cc(-c2ccnc(C#N)c2)c1NC(=O)NS(=O)(=O)C1CN(Cc2cccnc2)C1 ; COc1cc(-c2ccc3c(c2NC(=O)NS(=O)(=O)C2CN(Cc4cccnc4)C2)CCC3)ccn1 ; O=C(Nc1c(-c2ccncc2)cc(F)c2c1CCC2)NS(=O)(=O)C1CN(Cc2cccnc2)C1 ; CC(C)c1cc(F)cc(-c2ccnc(C#N)c2)c1NC(=O)NS(=O)(=O)c1ccc(=O)n(C(C)C)c1 ; COc1cc(-c2cc(F)cc(C(C)C)c2NC(=O)NS(=O)(=O)c2ccc(=O)n(C(C)C)c2)ccn1]

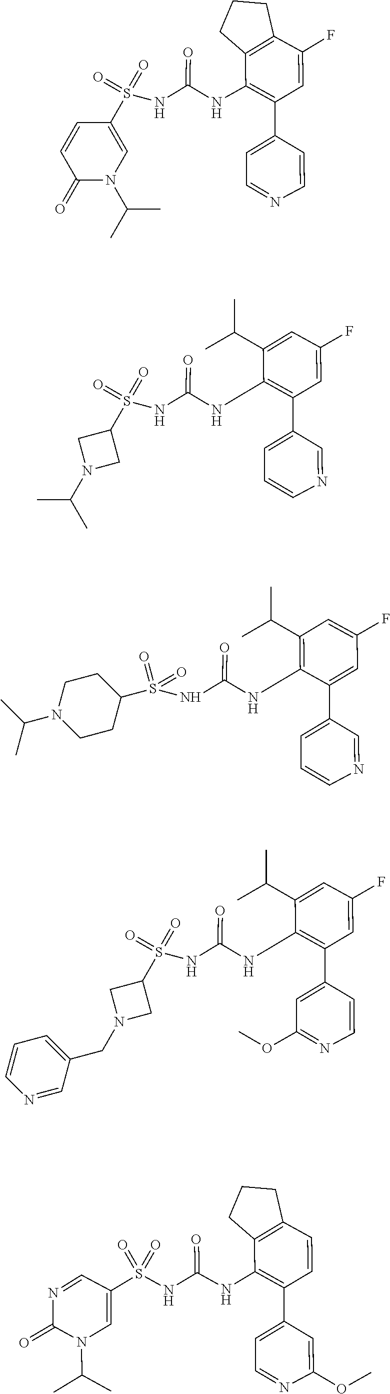 [SMILES: CC(C)n1cc(S(=O)(=O)NC(=O)Nc2c(-c3ccncc3)cc(F)c3c2CCC3)ccc1=O ; CC(C)c1cc(F)cc(-c2cccnc2)c1NC(=O)NS(=O)(=O)C1CN(C(C)C)C1 ; CC(C)c1cc(F)cc(-c2cccnc2)c1NC(=O)NS(=O)(=O)C1CCN(C(C)C)CC1 ; COc1cc(-c2cc(F)cc(C(C)C)c2NC(=O)NS(=O)(=O)C2CN(Cc3cccnc3)C2)ccn1 ; COc1cc(-c2ccc3c(c2NC(=O)NS(=O)(=O)c2cnc(=O)n(C(C)C)c2)CCC3)ccn1]

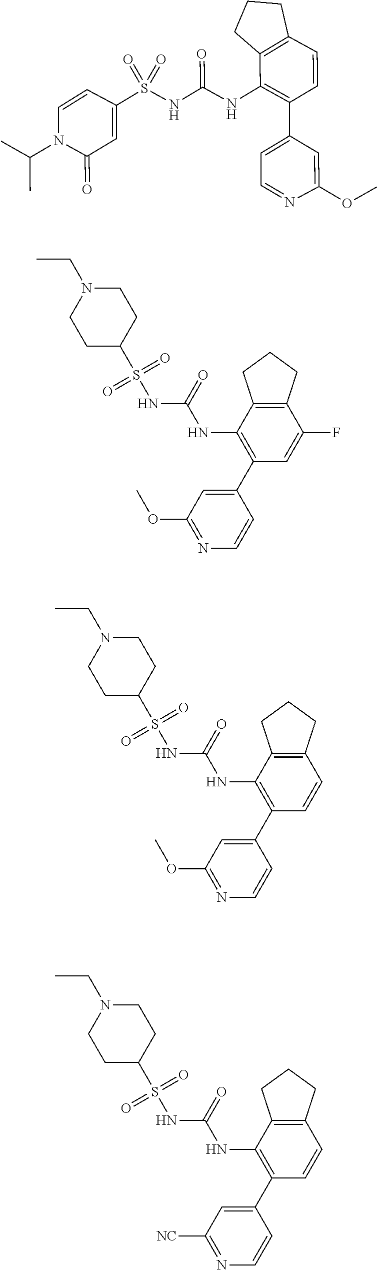

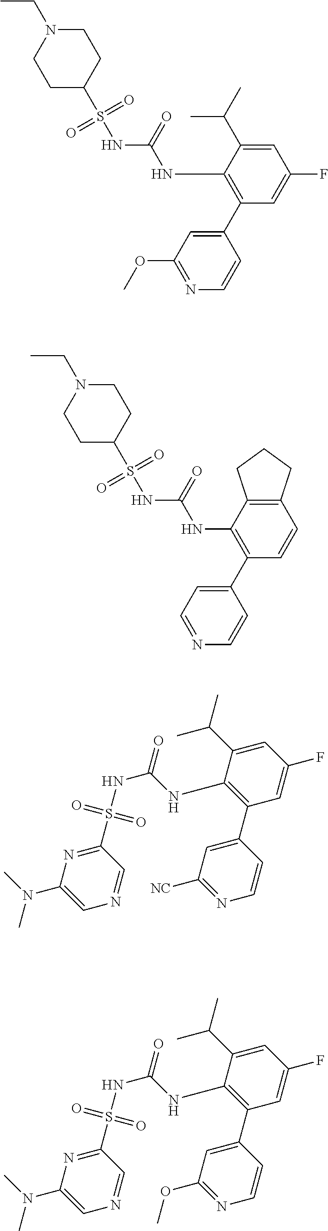 [SMILES: CCN1CCC(S(=O)(=O)NC(=O)Nc2c(-c3ccnc(OC)c3)cc(F)cc2C(C)C)CC1 ; CCN1CCC(S(=O)(=O)NC(=O)Nc2c(-c3ccncc3)ccc3c2CCC3)CC1 ; CC(C)c1cc(F)cc(-c2ccnc(C#N)c2)c1NC(=O)NS(=O)(=O)c1cncc(N(C)C)n1 ; COc1cc(-c2cc(F)cc(C(C)C)c2NC(=O)NS(=O)(=O)c2cncc(N(C)C)n2)ccn1]

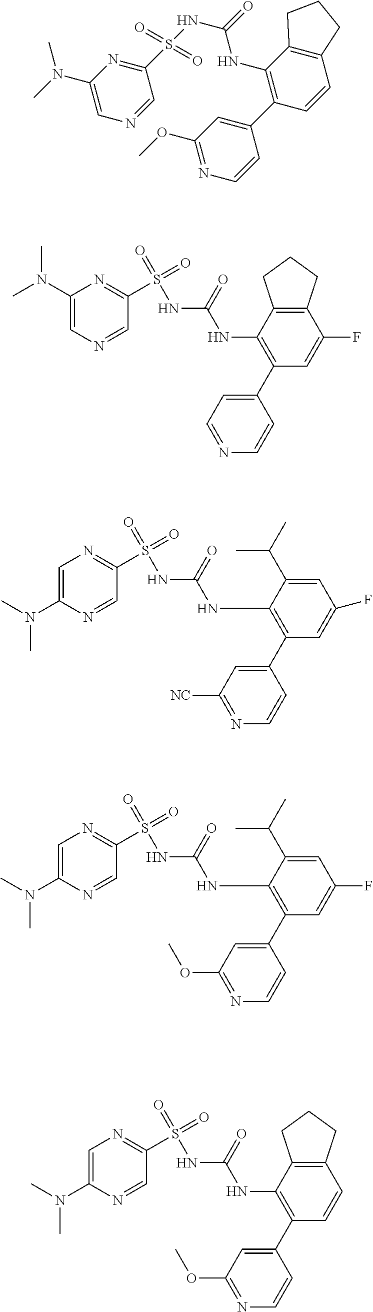 [SMILES: COc1cc(-c2ccc3c(c2NC(=O)NS(=O)(=O)c2cncc(N(C)C)n2)CCC3)ccn1 ; CN(C)c1cncc(S(=O)(=O)NC(=O)Nc2c(-c3ccncc3)cc(F)c3c2CCC3)n1 ; CC(C)c1cc(F)cc(-c2ccnc(C#N)c2)c1NC(=O)NS(=O)(=O)c1cnc(N(C)C)cn1 ; COc1cc(-c2cc(F)cc(C(C)C)c2NC(=O)NS(=O)(=O)c2cnc(N(C)C)cn2)ccn1 ; COc1cc(-c2ccc3c(c2NC(=O)NS(=O)(=O)c2cnc(N(C)C)cn2)CCC3)ccn1]

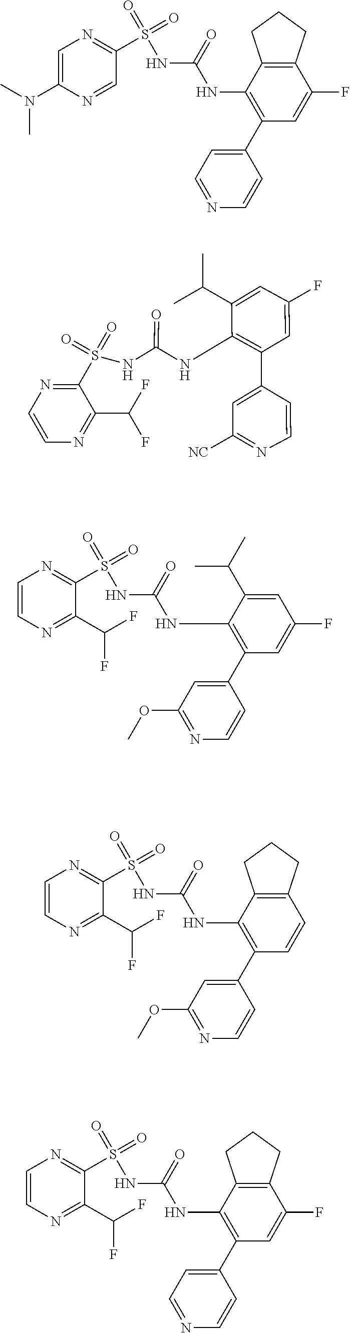 [SMILES: CN(C)c1cnc(S(=O)(=O)NC(=O)Nc2c(-c3ccncc3)cc(F)c3c2CCC3)cn1 ; CC(C)c1cc(F)cc(-c2ccnc(C#N)c2)c1NC(=O)NS(=O)(=O)c1nccnc1C(F)F ; COc1cc(-c2cc(F)cc(C(C)C)c2NC(=O)NS(=O)(=O)c2nccnc2C(F)F)ccn1 ; COc1cc(-c2ccc3c(c2NC(=O)NS(=O)(=O)c2nccnc2C(F)F)CCC3)ccn1 ; O=C(Nc1c(-c2ccncc2)cc(F)c2c1CCC2)NS(=O)(=O)c1nccnc1C(F)F]

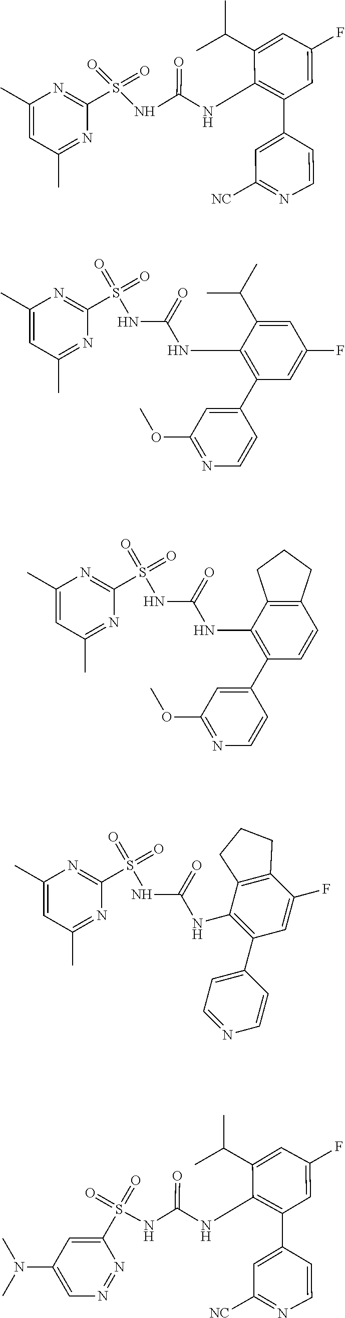 [SMILES: Cc1cc(C)nc(S(=O)(=O)NC(=O)Nc2c(-c3ccnc(C#N)c3)cc(F)cc2C(C)C)n1 ; COc1cc(-c2cc(F)cc(C(C)C)c2NC(=O)NS(=O)(=O)c2nc(C)cc(C)n2)ccn1 ; COc1cc(-c2ccc3c(c2NC(=O)NS(=O)(=O)c2nc(C)cc(C)n2)CCC3)ccn1 ; Cc1cc(C)nc(S(=O)(=O)NC(=O)Nc2c(-c3ccncc3)cc(F)c3c2CCC3)n1 ; CC(C)c1cc(F)cc(-c2ccnc(C#N)c2)c1NC(=O)NS(=O)(=O)c1cc(N(C)C)cnn1]

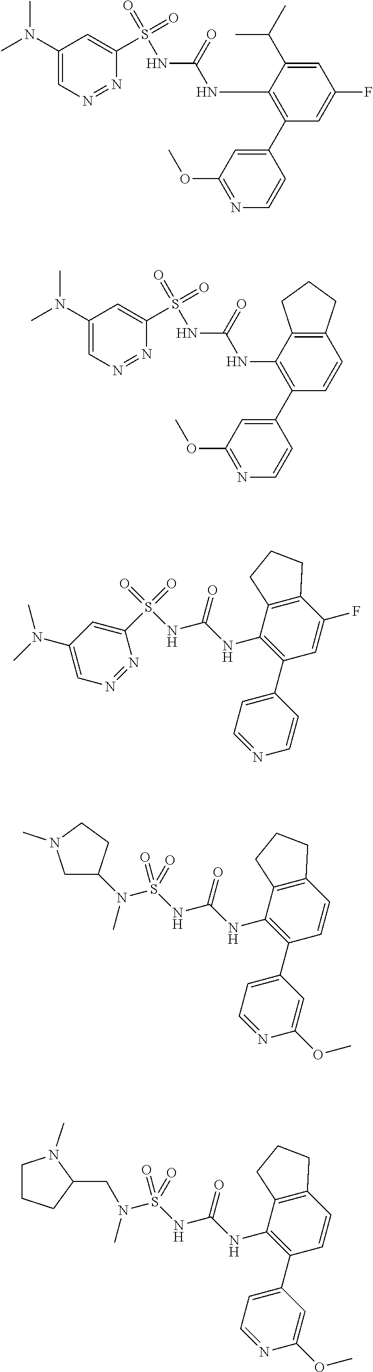

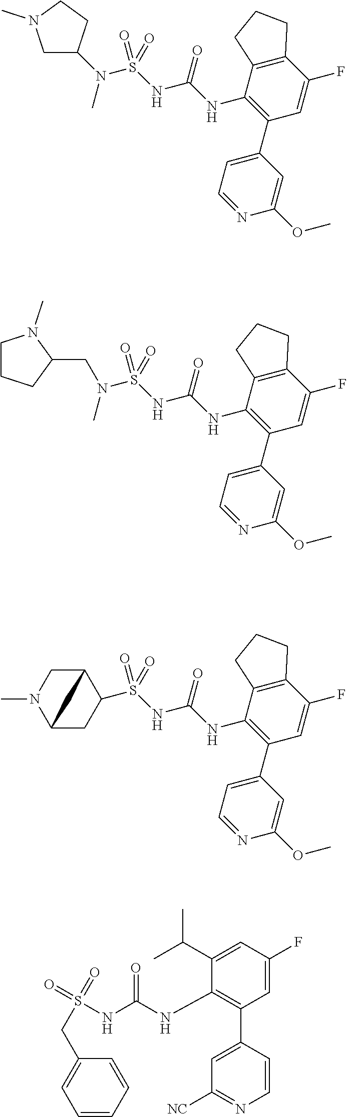 [SMILES: COc1cc(-c2cc(F)c3c(c2NC(=O)NS(=O)(=O)N(C)C2CCN(C)C2)CCC3)ccn1 ; COc1cc(-c2cc(F)c3c(c2NC(=O)NS(=O)(=O)N(C)CC2CCCN2C)CCC3)ccn1 ; COc1cc(-c2cc(F)c3c(c2NC(=O)NS(=O)(=O)C2C[C@H]4C[C@@H]2CN4C)CCC3)ccn1 ; CC(C)c1cc(F)cc(-c2ccnc(C#N)c2)c1NC(=O)NS(=O)(=O)Cc1ccccc1]

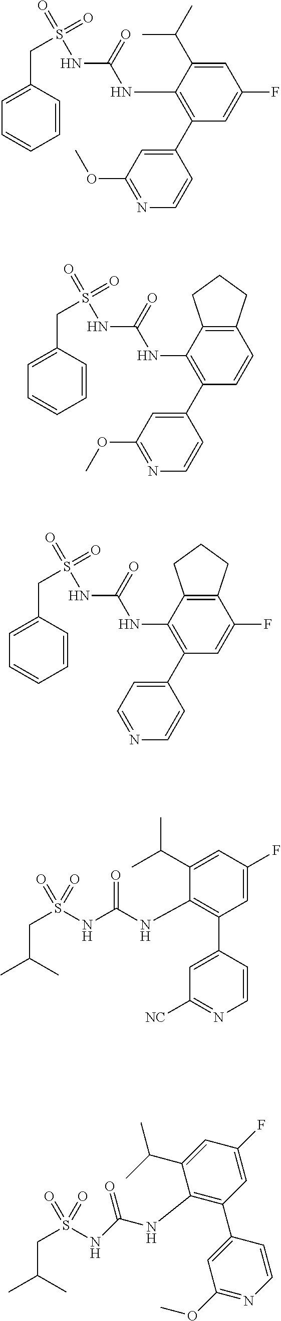 [SMILES: COc1cc(-c2cc(F)cc(C(C)C)c2NC(=O)NS(=O)(=O)Cc2ccccc2)ccn1 ; COc1cc(-c2ccc3c(c2NC(=O)NS(=O)(=O)Cc2ccccc2)CCC3)ccn1 ; O=C(Nc1c(-c2ccncc2)cc(F)c2c1CCC2)NS(=O)(=O)Cc1ccccc1 ; CC(C)CS(=O)(=O)NC(=O)Nc1c(-c2ccnc(C#N)c2)cc(F)cc1C(C)C ; COc1cc(-c2cc(F)cc(C(C)C)c2NC(=O)NS(=O)(=O)CC(C)C)ccn1]

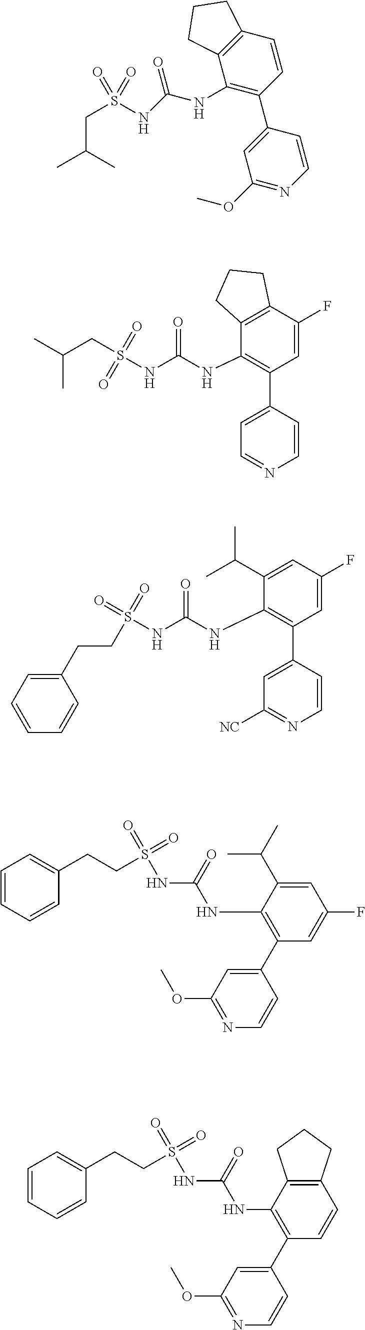 [SMILES: COc1cc(-c2ccc3c(c2NC(=O)NS(=O)(=O)CC(C)C)CCC3)ccn1 ; CC(C)CS(=O)(=O)NC(=O)Nc1c(-c2ccncc2)cc(F)c2c1CCC2 ; CC(C)c1cc(F)cc(-c2ccnc(C#N)c2)c1NC(=O)NS(=O)(=O)CCc1ccccc1 ; COc1cc(-c2cc(F)cc(C(C)C)c2NC(=O)NS(=O)(=O)CCc2ccccc2)ccn1 ; COc1cc(-c2ccc3c(c2NC(=O)NS(=O)(=O)CCc2ccccc2)CCC3)ccn1]

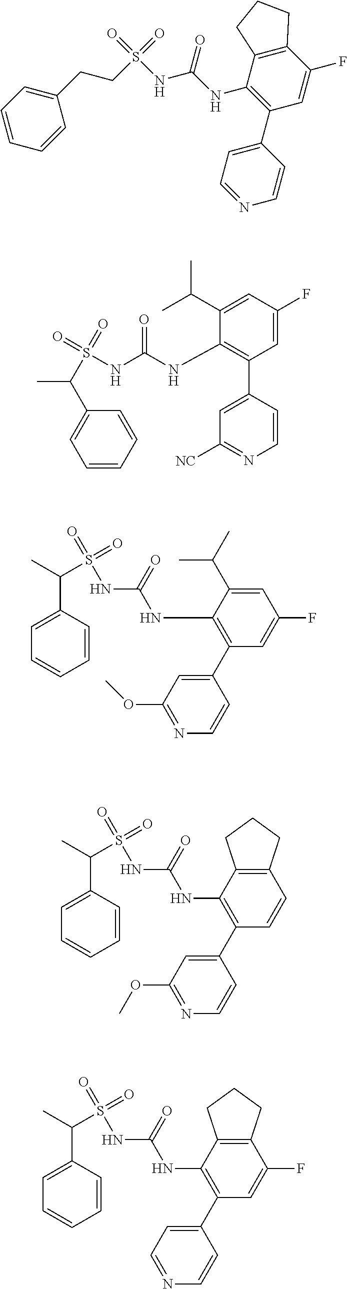 [SMILES: O=C(Nc1c(-c2ccncc2)cc(F)c2c1CCC2)NS(=O)(=O)CCc1ccccc1 ; CC(C)c1cc(F)cc(-c2ccnc(C#N)c2)c1NC(=O)NS(=O)(=O)C(C)c1ccccc1 ; COc1cc(-c2cc(F)cc(C(C)C)c2NC(=O)NS(=O)(=O)C(C)c2ccccc2)ccn1 ; COc1cc(-c2ccc3c(c2NC(=O)NS(=O)(=O)C(C)c2ccccc2)CCC3)ccn1 ; CC(c1ccccc1)S(=O)(=O)NC(=O)Nc1c(-c2ccncc2)cc(F)c2c1CCC2]

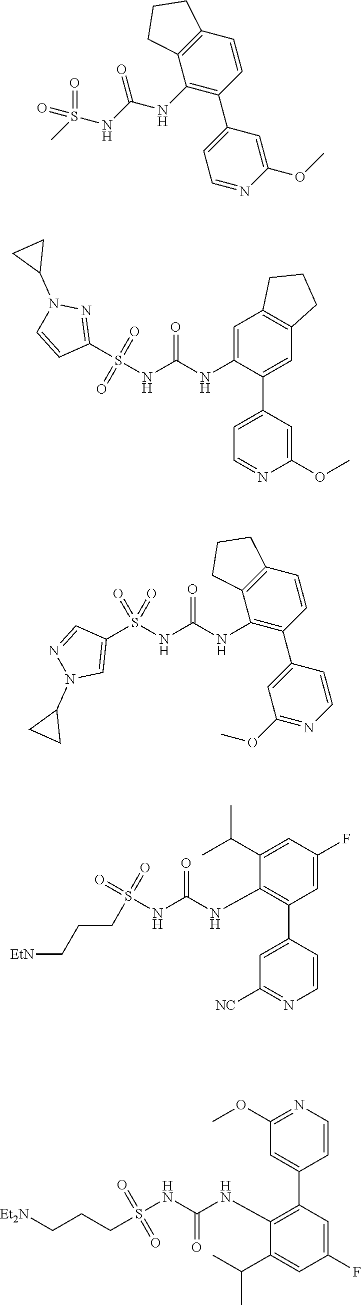

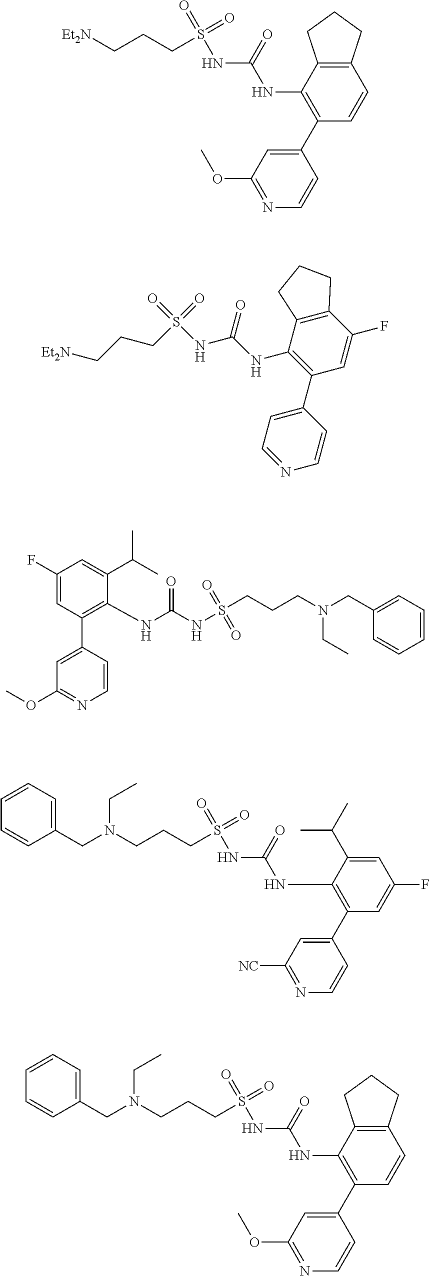

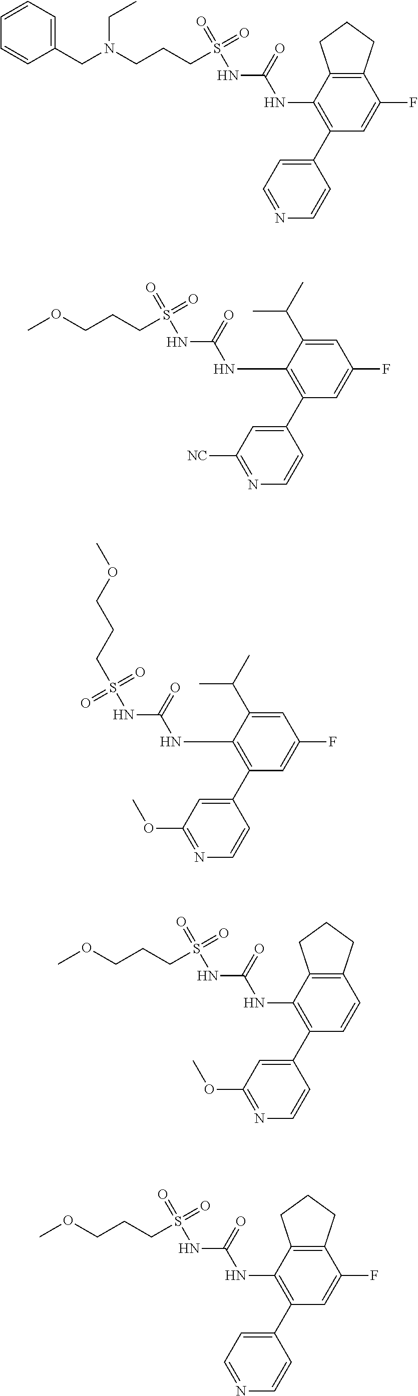 [SMILES: CCN(CCCS(=O)(=O)NC(=O)Nc1c(-c2ccncc2)cc(F)c2c1CCC2)Cc1ccccc1 ; COCCCS(=O)(=O)NC(=O)Nc1c(-c2ccnc(C#N)c2)cc(F)cc1C(C)C ; COCCCS(=O)(=O)NC(=O)Nc1c(-c2ccnc(OC)c2)cc(F)cc1C(C)C ; COCCCS(=O)(=O)NC(=O)Nc1c(-c2ccnc(OC)c2)ccc2c1CCC2 ; COCCCS(=O)(=O)NC(=O)Nc1c(-c2ccncc2)cc(F)c2c1CCC2]

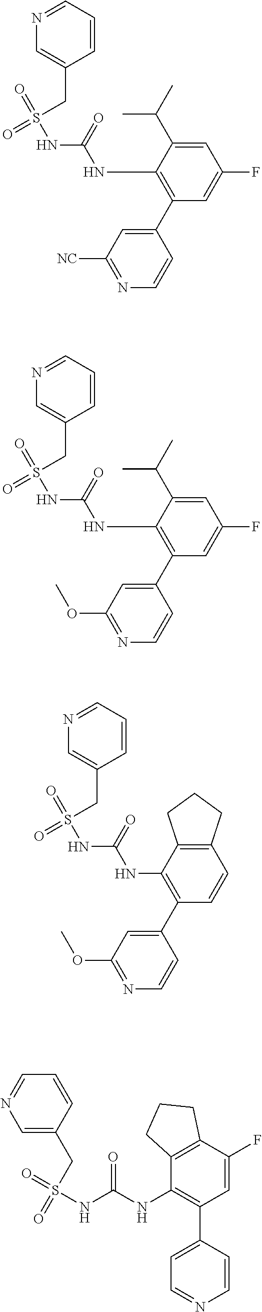 [SMILES: CC(C)c1cc(F)cc(-c2ccnc(C#N)c2)c1NC(=O)NS(=O)(=O)Cc1cccnc1 ; COc1cc(-c2cc(F)cc(C(C)C)c2NC(=O)NS(=O)(=O)Cc2cccnc2)ccn1 ; COc1cc(-c2ccc3c(c2NC(=O)NS(=O)(=O)Cc2cccnc2)CCC3)ccn1 ; O=C(Nc1c(-c2ccncc2)cc(F)c2c1CCC2)NS(=O)(=O)Cc1cccnc1]

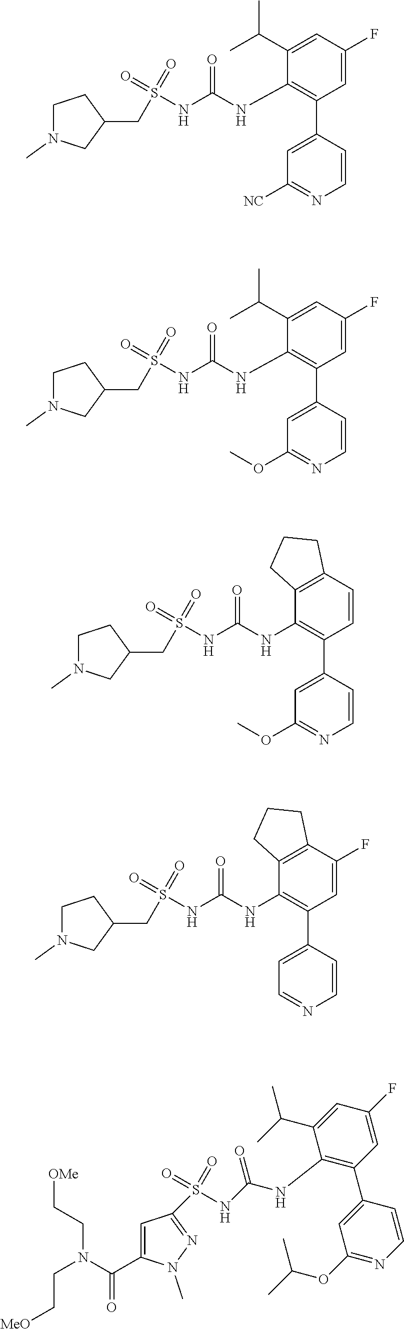 [SMILES: CC(C)c1cc(F)cc(-c2ccnc(C#N)c2)c1NC(=O)NS(=O)(=O)CC1CCN(C)C1 ; COc1cc(-c2cc(F)cc(C(C)C)c2NC(=O)NS(=O)(=O)CC2CCN(C)C2)ccn1 ; COc1cc(-c2ccc3c(c2NC(=O)NS(=O)(=O)CC2CCN(C)C2)CCC3)ccn1 ; CN1CCC(CS(=O)(=O)NC(=O)Nc2c(-c3ccncc3)cc(F)c3c2CCC3)C1 ; COCCN(CCOC)C(=O)c1cc(S(=O)(=O)NC(=O)Nc2c(-c3ccnc(OC(C)C)c3)cc(F)cc2C(C)C)nn1C]

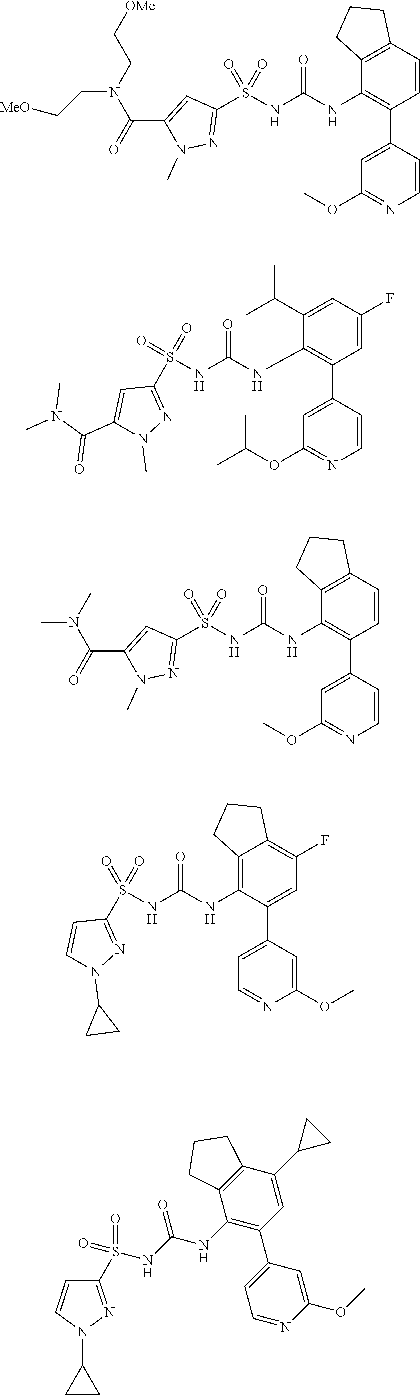 [SMILES: COCCN(CCOC)C(=O)c1cc(S(=O)(=O)NC(=O)Nc2c(-c3ccnc(OC)c3)ccc3c2CCC3)nn1C ; CC(C)Oc1cc(-c2cc(F)cc(C(C)C)c2NC(=O)NS(=O)(=O)c2cc(C(=O)N(C)C)n(C)n2)ccn1 ; COc1cc(-c2ccc3c(c2NC(=O)NS(=O)(=O)c2cc(C(=O)N(C)C)n(C)n2)CCC3)ccn1 ; COc1cc(-c2cc(F)c3c(c2NC(=O)NS(=O)(=O)c2ccn(C4CC4)n2)CCC3)ccn1 ; COc1cc(-c2cc(C3CC3)c3c(c2NC(=O)NS(=O)(=O)c2ccn(C4CC4)n2)CCC3)ccn1]

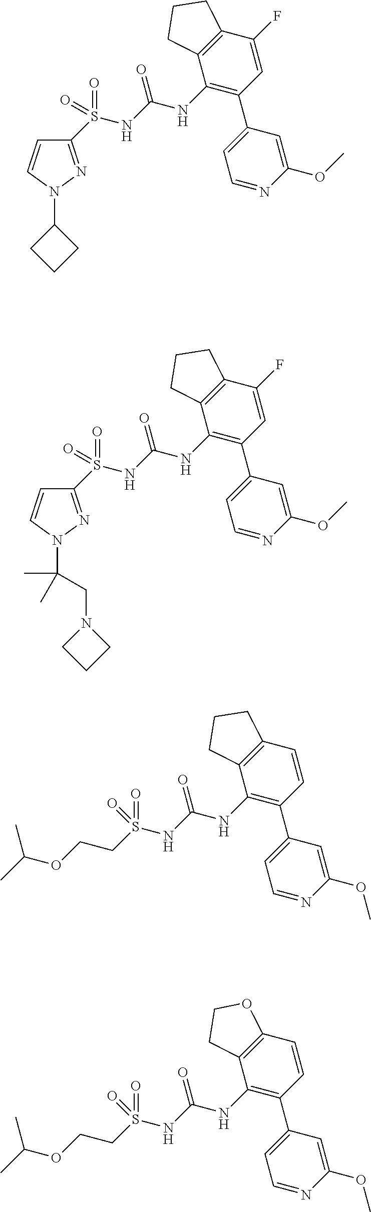

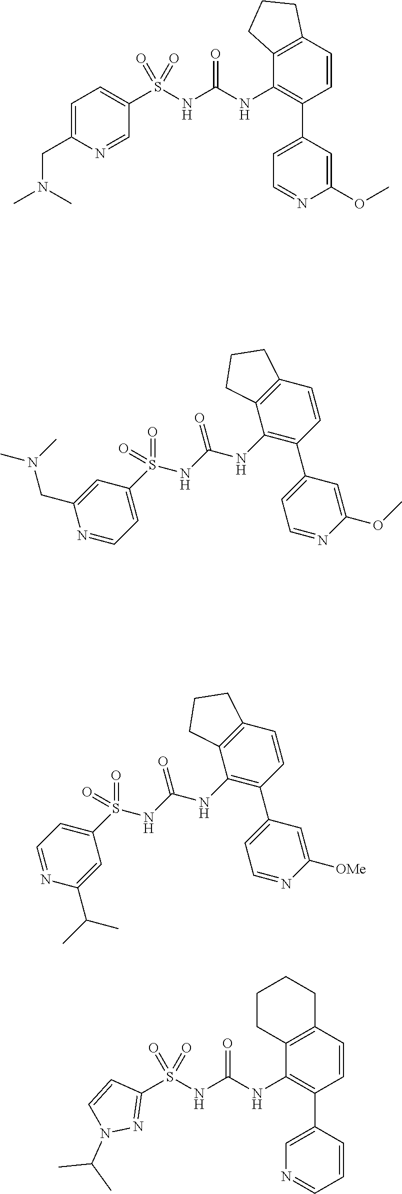

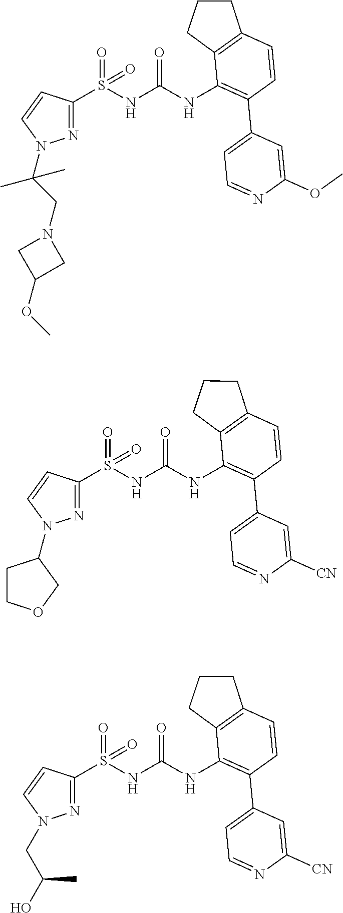 [SMILES: COc1cc(-c2ccc3c(c2NC(=O)NS(=O)(=O)c2ccn(C(C)(C)CN4CC(OC)C4)n2)CCC3)ccn1 ; N#Cc1cc(-c2ccc3c(c2NC(=O)NS(=O)(=O)c2ccn(C4CCOC4)n2)CCC3)ccn1 ; C[C@@H](O)Cn1ccc(S(=O)(=O)NC(=O)Nc2c(-c3ccnc(C#N)c3)ccc3c2CCC3)n1]

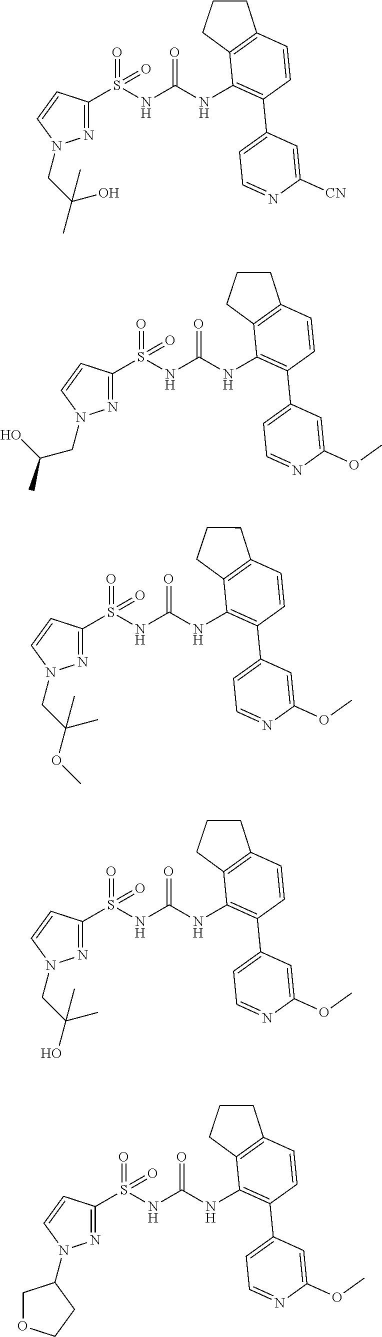 [SMILES: CC(C)(O)Cn1ccc(S(=O)(=O)NC(=O)Nc2c(-c3ccnc(C#N)c3)ccc3c2CCC3)n1 ; COc1cc(-c2ccc3c(c2NC(=O)NS(=O)(=O)c2ccn(C[C@@H](C)O)n2)CCC3)ccn1 ; COc1cc(-c2ccc3c(c2NC(=O)NS(=O)(=O)c2ccn(CC(C)(C)OC)n2)CCC3)ccn1 ; COc1cc(-c2ccc3c(c2NC(=O)NS(=O)(=O)c2ccn(CC(C)(C)O)n2)CCC3)ccn1 ; COc1cc(-c2ccc3c(c2NC(=O)NS(=O)(=O)c2ccn(C4CCOC4)n2)CCC3)ccn1]

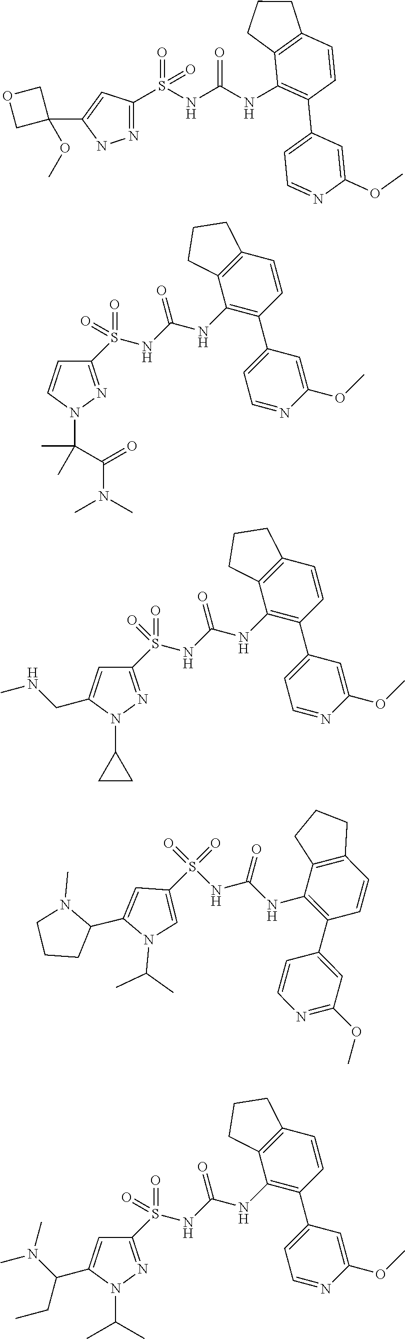

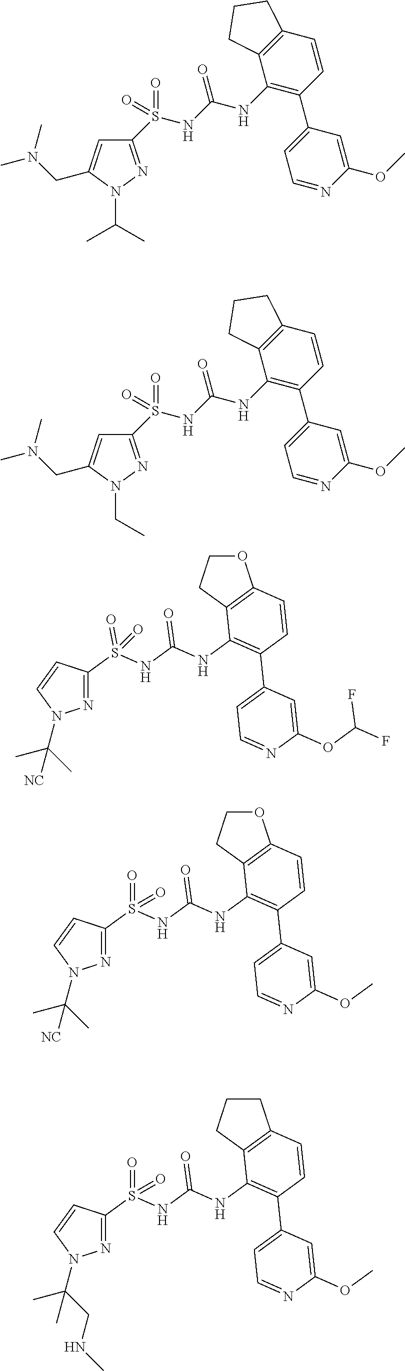

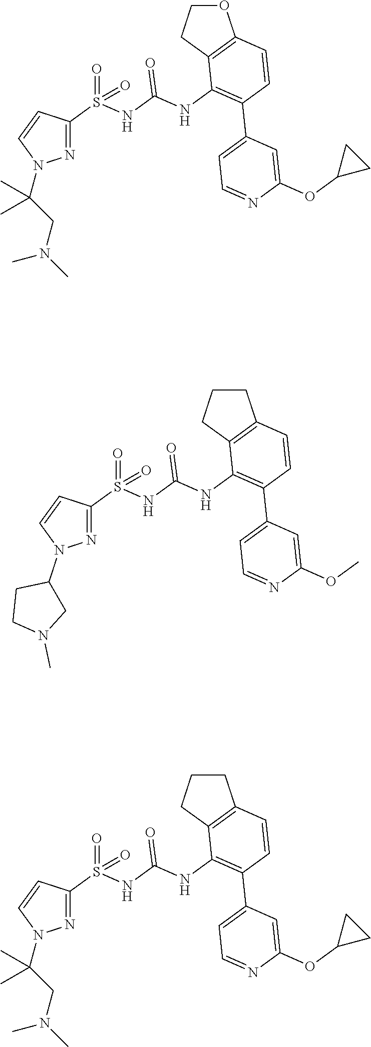

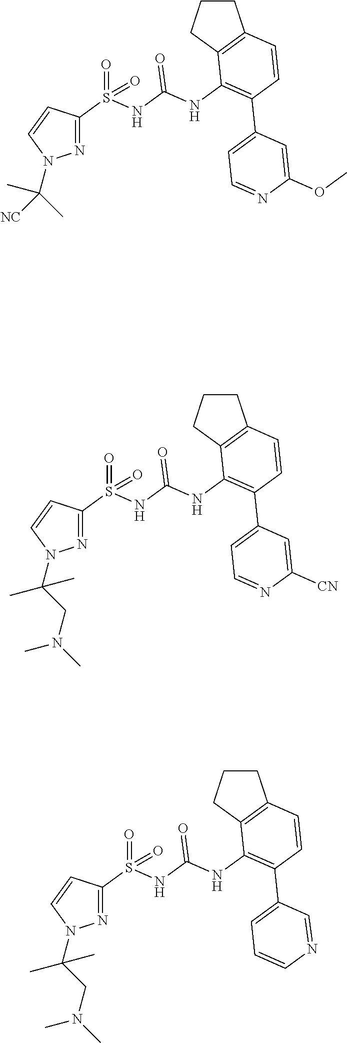

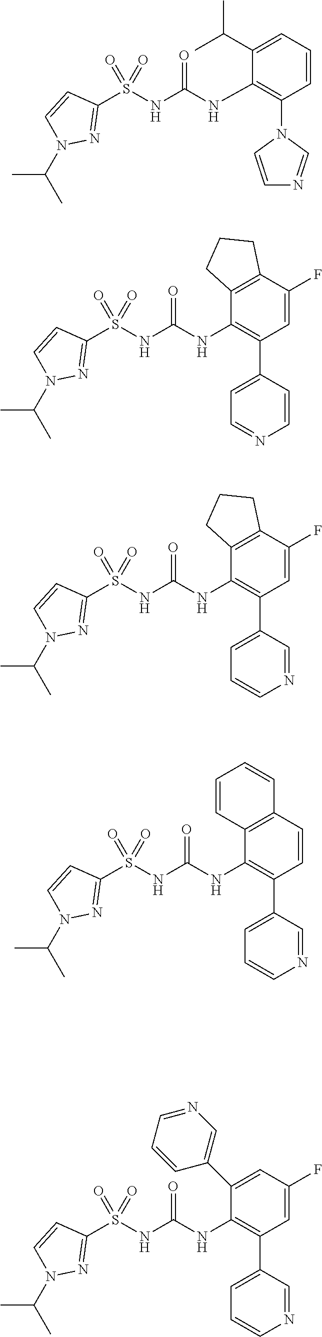 [SMILES: CC(C)c1cccc(-n2ccnc2)c1NC(=O)NS(=O)(=O)c1ccn(C(C)C)n1 ; CC(C)n1ccc(S(=O)(=O)NC(=O)Nc2c(-c3ccncc3)cc(F)c3c2CCC3)n1 ; CC(C)n1ccc(S(=O)(=O)NC(=O)Nc2c(-c3cccnc3)cc(F)c3c2CCC3)n1 ; CC(C)n1ccc(S(=O)(=O)NC(=O)Nc2c(-c3cccnc3)ccc3ccccc23)n1 ; CC(C)n1ccc(S(=O)(=O)NC(=O)Nc2c(-c3cccnc3)cc(F)cc2-c2cccnc2)n1]

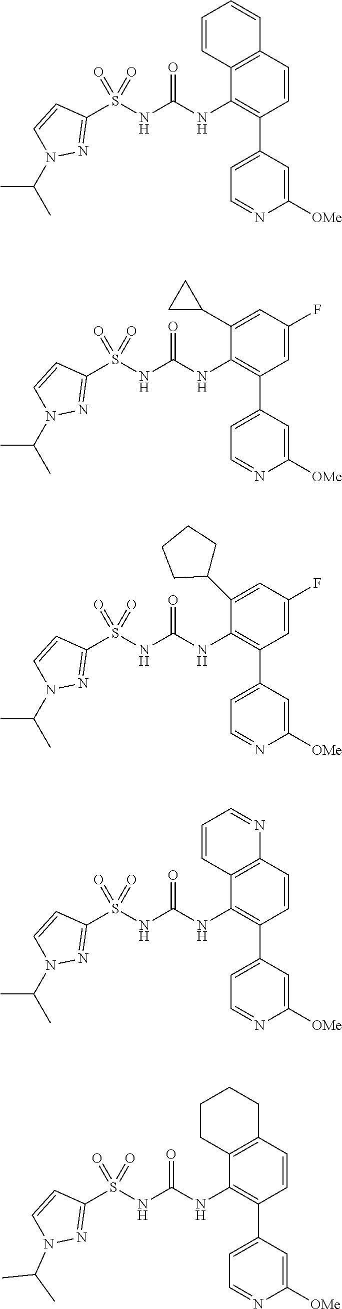 [SMILES: COc1cc(-c2ccc3ccccc3c2NC(=O)NS(=O)(=O)c2ccn(C(C)C)n2)ccn1 ; COc1cc(-c2cc(F)cc(C3CC3)c2NC(=O)NS(=O)(=O)c2ccn(C(C)C)n2)ccn1 ; COc1cc(-c2cc(F)cc(C3CCCC3)c2NC(=O)NS(=O)(=O)c2ccn(C(C)C)n2)ccn1 ; COc1cc(-c2ccc3ncccc3c2NC(=O)NS(=O)(=O)c2ccn(C(C)C)n2)ccn1 ; COc1cc(-c2ccc3c(c2NC(=O)NS(=O)(=O)c2ccn(C(C)C)n2)CCCC3)ccn1]

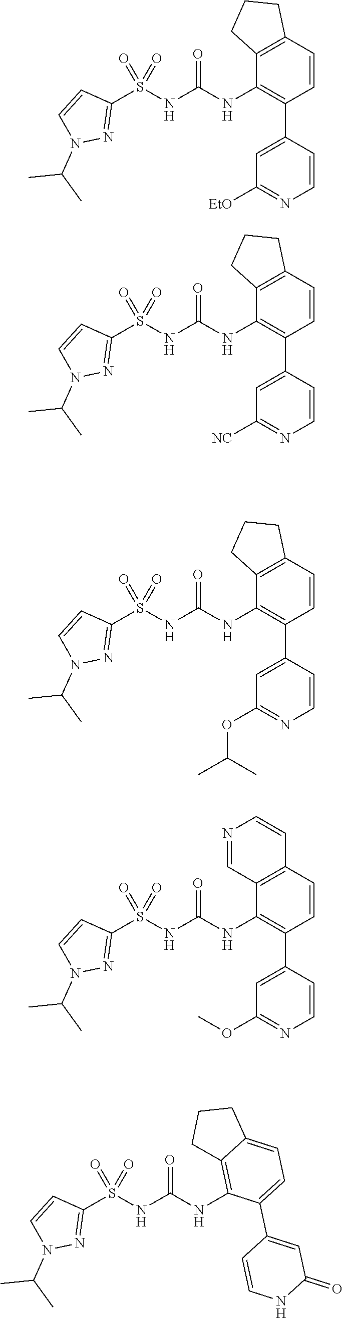

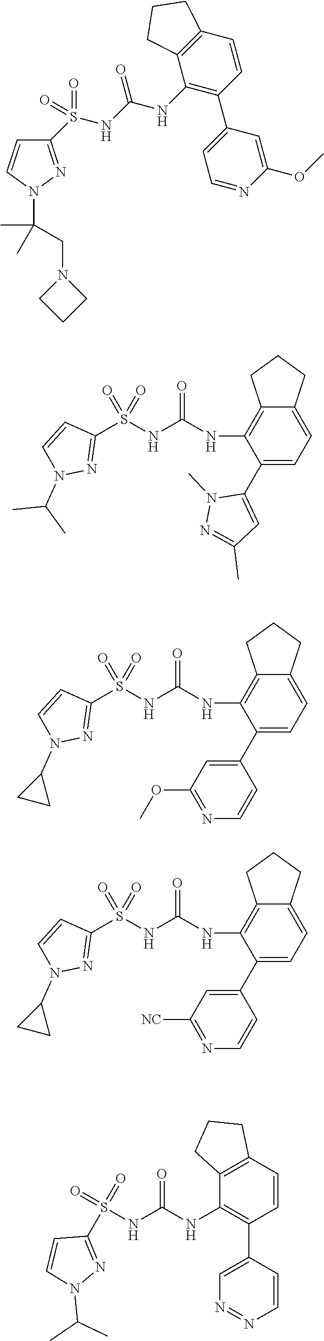

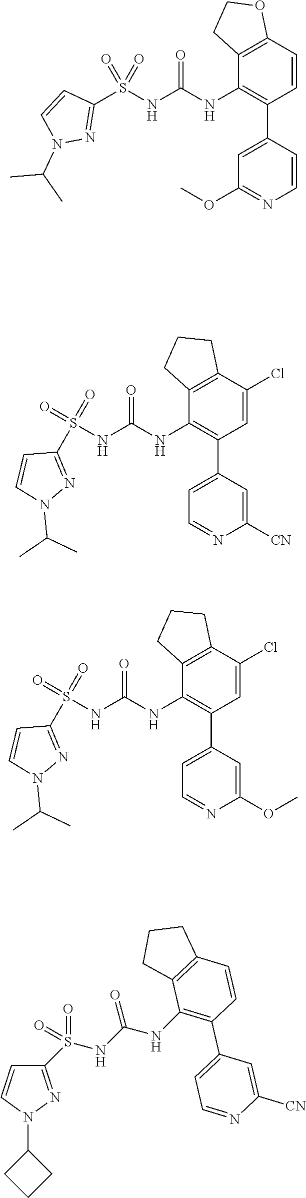 [SMILES: COc1cc(-c2ccc3c(c2NC(=O)NS(=O)(=O)c2ccn(C(C)C)n2)CCO3)ccn1 ; CC(C)n1ccc(S(=O)(=O)NC(=O)Nc2c(-c3ccnc(C#N)c3)cc(Cl)c3c2CCC3)n1 ; COc1cc(-c2cc(Cl)c3c(c2NC(=O)NS(=O)(=O)c2ccn(C(C)C)n2)CCC3)ccn1 ; N#Cc1cc(-c2ccc3c(c2NC(=O)NS(=O)(=O)c2ccn(C4CCC4)n2)CCC3)ccn1]

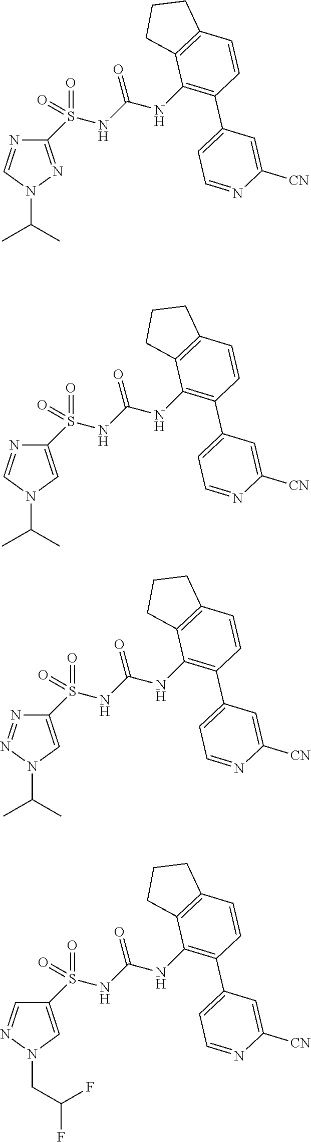 [SMILES: CC(C)n1cnc(S(=O)(=O)NC(=O)Nc2c(-c3ccnc(C#N)c3)ccc3c2CCC3)n1 ; CC(C)n1cnc(S(=O)(=O)NC(=O)Nc2c(-c3ccnc(C#N)c3)ccc3c2CCC3)c1 ; CC(C)n1cc(S(=O)(=O)NC(=O)Nc2c(-c3ccnc(C#N)c3)ccc3c2CCC3)nn1 ; N#Cc1cc(-c2ccc3c(c2NC(=O)NS(=O)(=O)c2cnn(CC(F)F)c2)CCC3)ccn1]

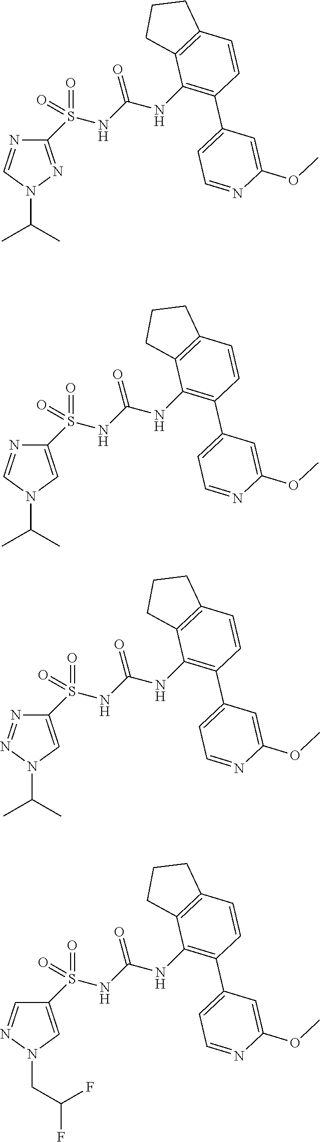

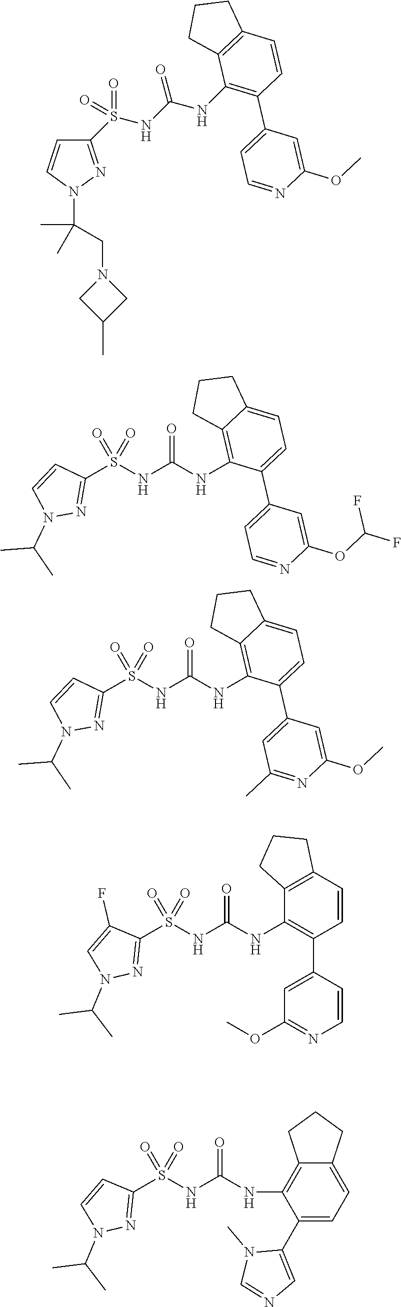

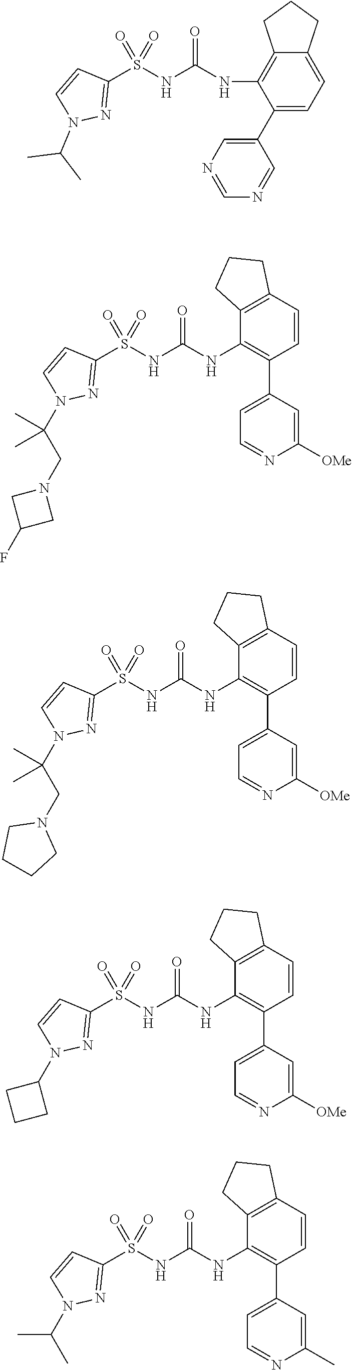 [SMILES: CC(C)n1ccc(S(=O)(=O)NC(=O)Nc2c(-c3cncnc3)ccc3c2CCC3)n1 ; COc1cc(-c2ccc3c(c2NC(=O)NS(=O)(=O)c2ccn(C(C)(C)CN4CC(F)C4)n2)CCC3)ccn1 ; COc1cc(-c2ccc3c(c2NC(=O)NS(=O)(=O)c2ccn(C(C)(C)CN4CCCC4)n2)CCC3)ccn1 ; COc1cc(-c2ccc3c(c2NC(=O)NS(=O)(=O)c2ccn(C4CCC4)n2)CCC3)ccn1 ; Cc1cc(-c2ccc3c(c2NC(=O)NS(=O)(=O)c2ccn(C(C)C)n2)CCC3)ccn1]

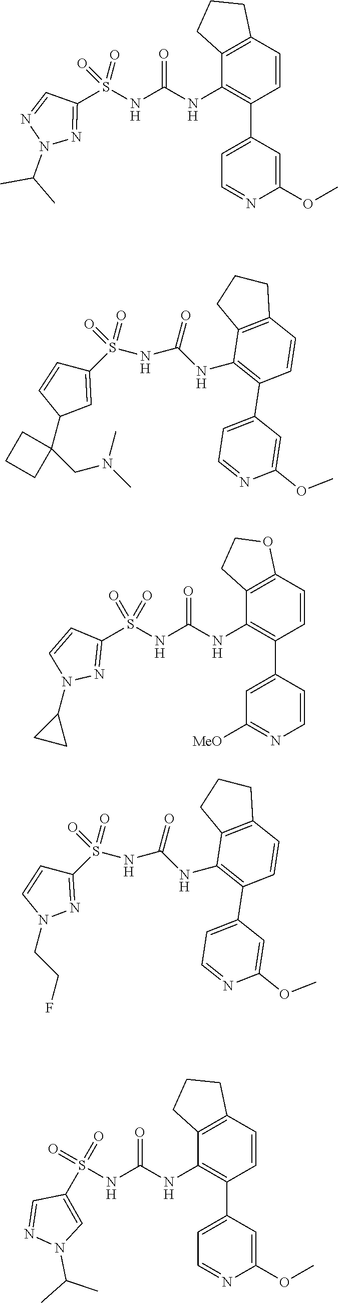

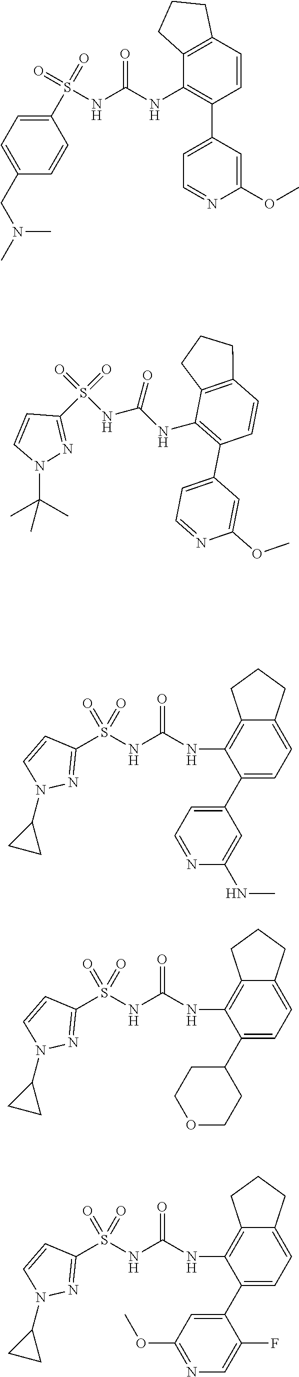

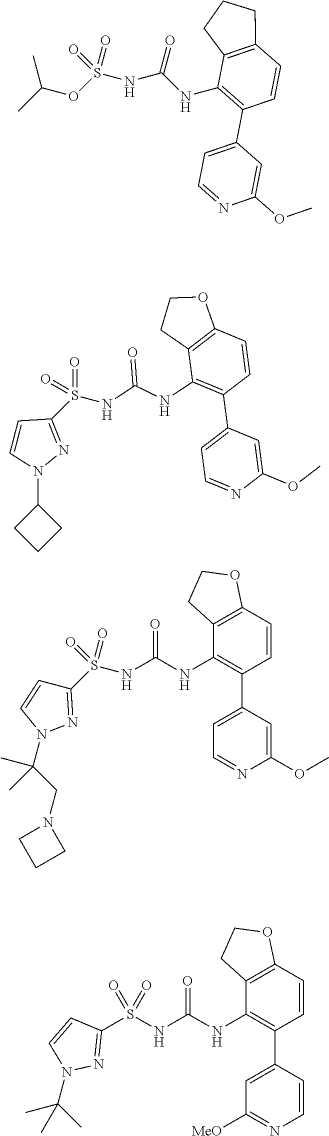

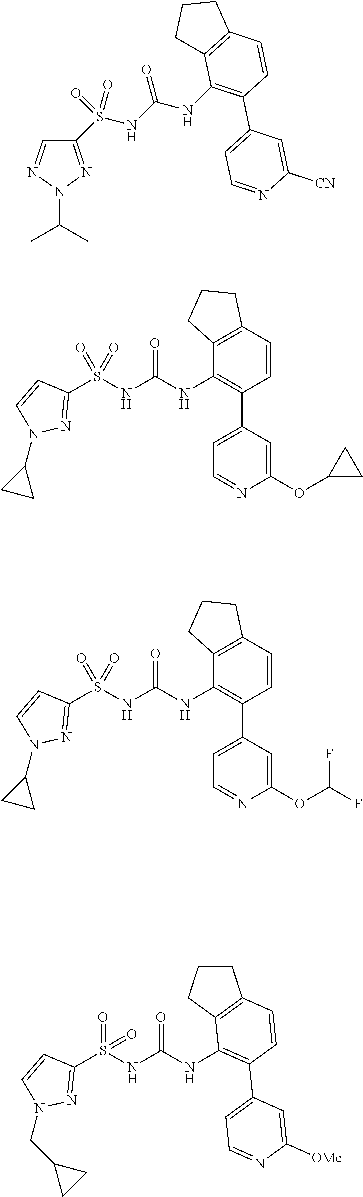 [SMILES: CC(C)n1ncc(S(=O)(=O)NC(=O)Nc2c(-c3ccnc(C#N)c3)ccc3c2CCC3)n1 ; O=C(Nc1c(-c2ccnc(OC3CC3)c2)ccc2c1CCC2)NS(=O)(=O)c1ccn(C2CC2)n1 ; O=C(Nc1c(-c2ccnc(OC(F)F)c2)ccc2c1CCC2)NS(=O)(=O)c1ccn(C2CC2)n1 ; COc1cc(-c2ccc3c(c2NC(=O)NS(=O)(=O)c2ccn(CC4CC4)n2)CCC3)ccn1]

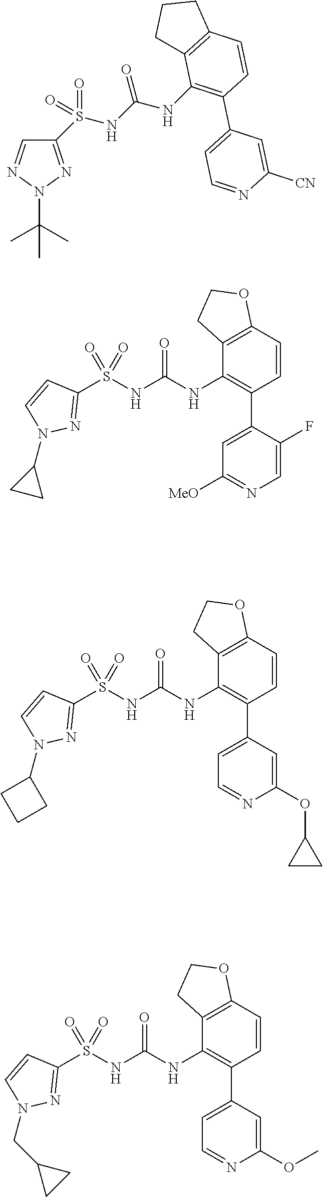 [SMILES: CC(C)(C)n1ncc(S(=O)(=O)NC(=O)Nc2c(-c3ccnc(C#N)c3)ccc3c2CCC3)n1 ; COc1cc(-c2ccc3c(c2NC(=O)NS(=O)(=O)c2ccn(C4CC4)n2)CCO3)c(F)cn1 ; O=C(Nc1c(-c2ccnc(OC3CC3)c2)ccc2c1CCO2)NS(=O)(=O)c1ccn(C2CCC2)n1 ; COc1cc(-c2ccc3c(c2NC(=O)NS(=O)(=O)c2ccn(CC4CC4)n2)CCO3)ccn1]

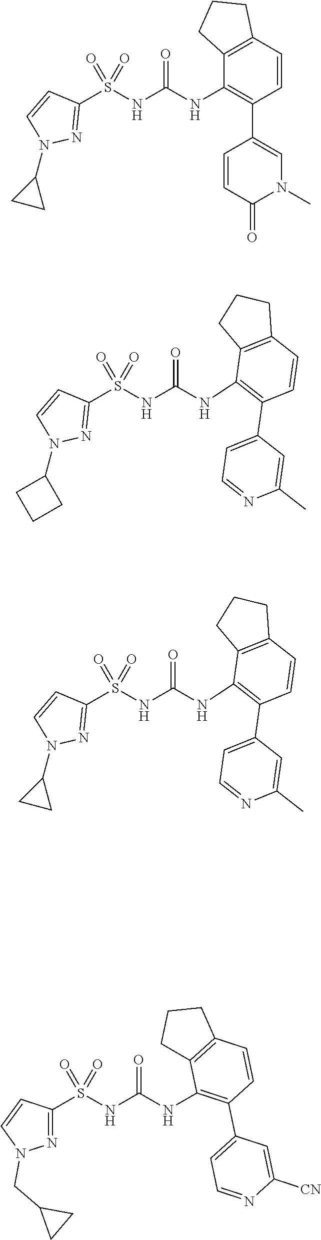 [SMILES: Cn1cc(-c2ccc3c(c2NC(=O)NS(=O)(=O)c2ccn(C4CC4)n2)CCC3)ccc1=O ; Cc1cc(-c2ccc3c(c2NC(=O)NS(=O)(=O)c2ccn(C4CCC4)n2)CCC3)ccn1 ; Cc1cc(-c2ccc3c(c2NC(=O)NS(=O)(=O)c2ccn(C4CC4)n2)CCC3)ccn1 ; N#Cc1cc(-c2ccc3c(c2NC(=O)NS(=O)(=O)c2ccn(CC4CC4)n2)CCC3)ccn1]

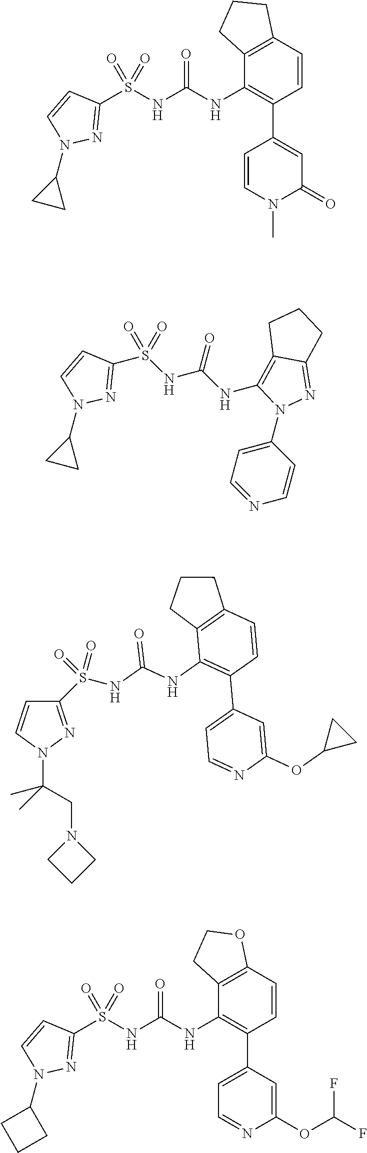 [SMILES: Cn1ccc(-c2ccc3c(c2NC(=O)NS(=O)(=O)c2ccn(C4CC4)n2)CCC3)cc1=O ; O=C(Nc1c2c(nn1-c1ccncc1)CCC2)NS(=O)(=O)c1ccn(C2CC2)n1 ; CC(C)(CN1CCC1)n1ccc(S(=O)(=O)NC(=O)Nc2c(-c3ccnc(OC4CC4)c3)ccc3c2CCC3)n1 ; O=C(Nc1c(-c2ccnc(OC(F)F)c2)ccc2c1CCO2)NS(=O)(=O)c1ccn(C2CCC2)n1]

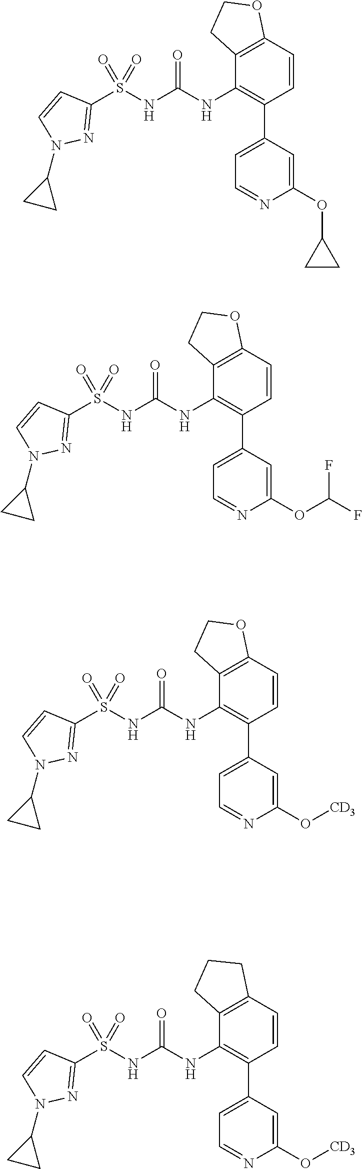 [SMILES: O=C(Nc1c(-c2ccnc(OC3CC3)c2)ccc2c1CCO2)NS(=O)(=O)c1ccn(C2CC2)n1 ; O=C(Nc1c(-c2ccnc(OC(F)F)c2)ccc2c1CCO2)NS(=O)(=O)c1ccn(C2CC2)n1 ; [2H]C([2H])([2H])Oc1cc(-c2ccc3c(c2NC(=O)NS(=O)(=O)c2ccn(C4CC4)n2)CCO3)ccn1 ; [2H]C([2H])([2H])Oc1cc(-c2ccc3c(c2NC(=O)NS(=O)(=O)c2ccn(C4CC4)n2)CCC3)ccn1]

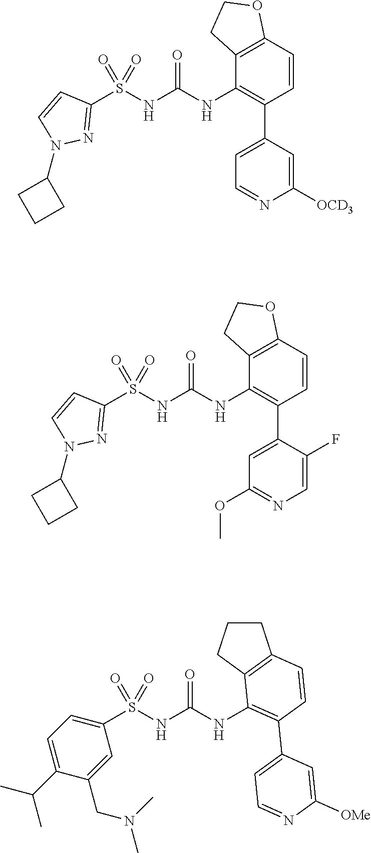

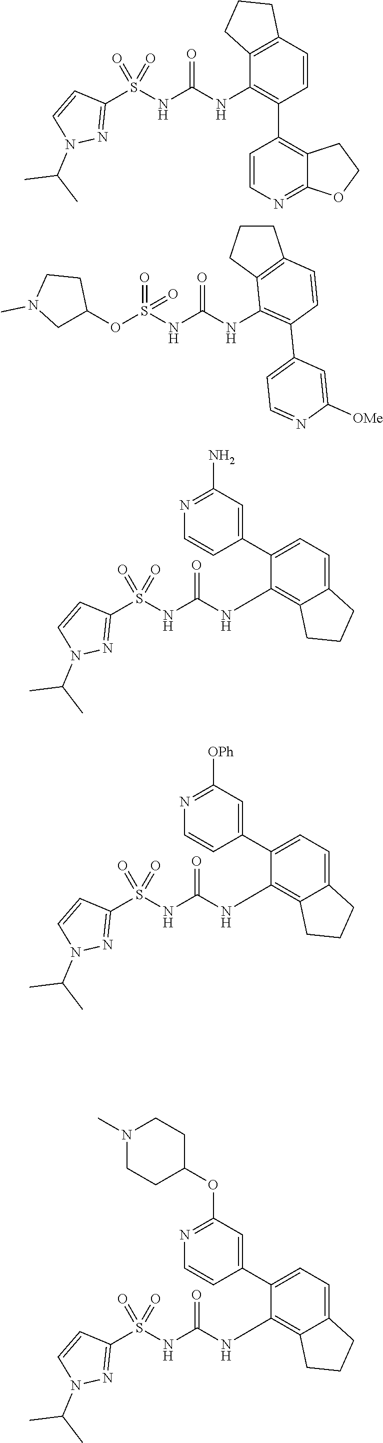

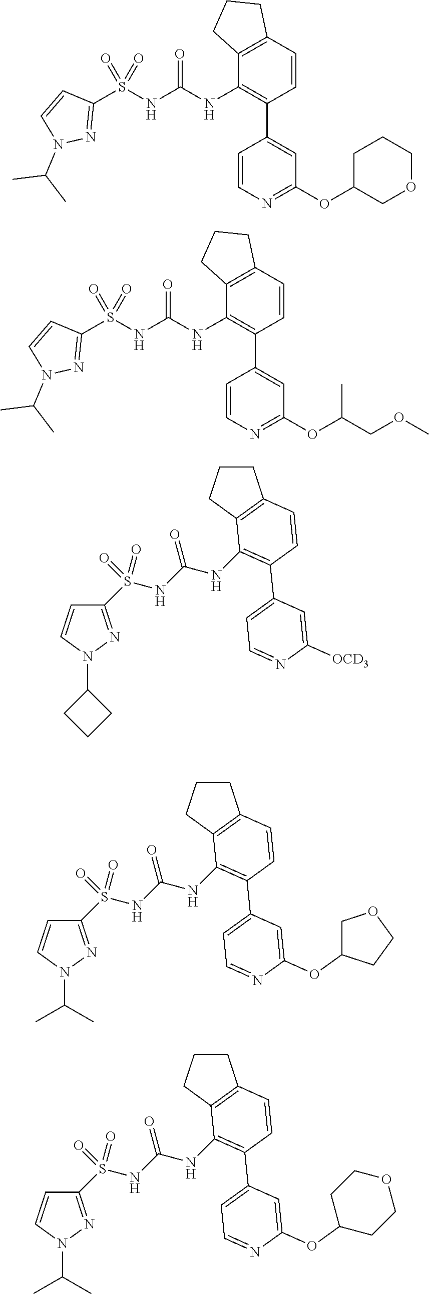

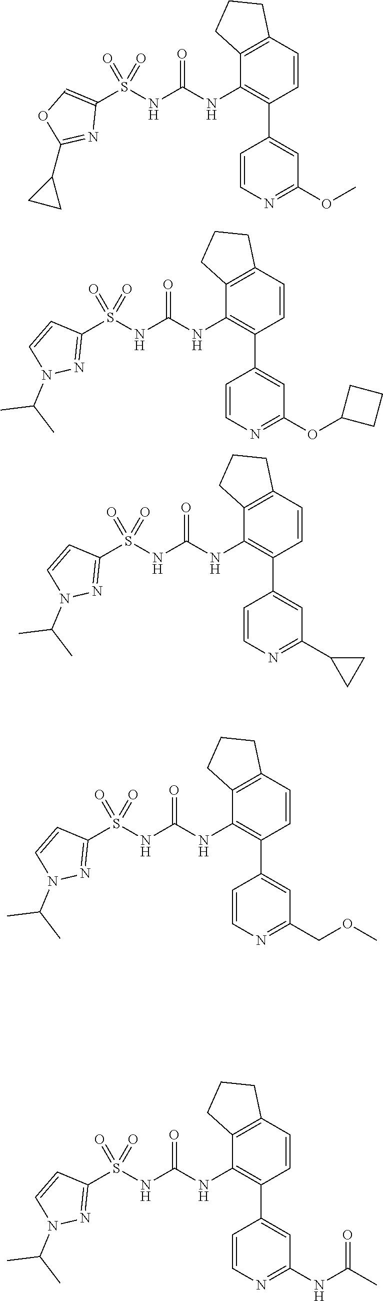

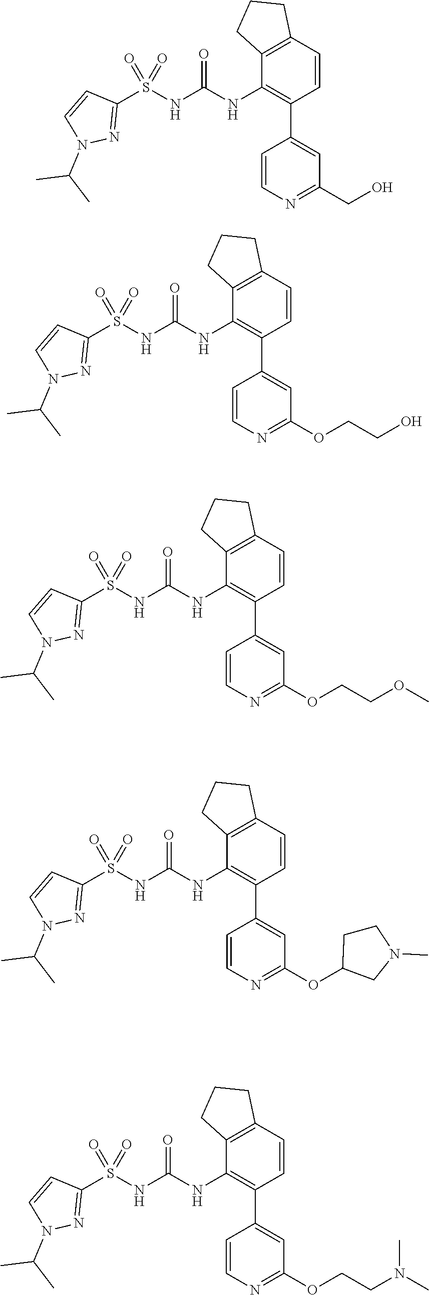

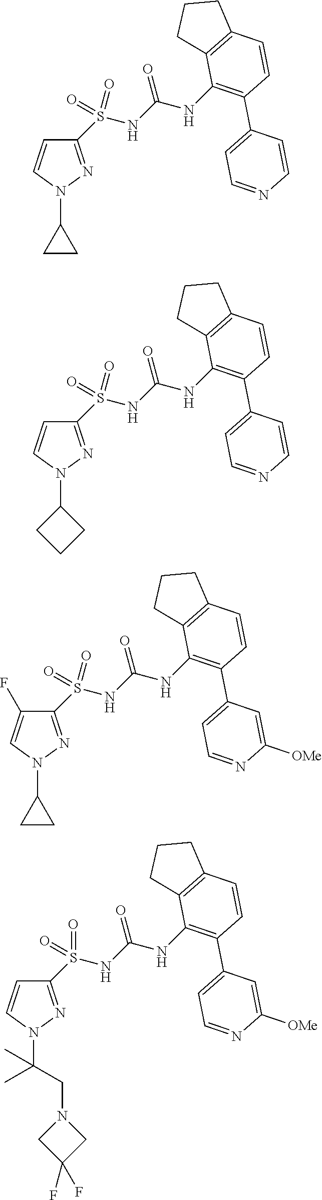 [SMILES: O=C(Nc1c(-c2ccncc2)ccc2c1CCC2)NS(=O)(=O)c1ccn(C2CC2)n1 ; O=C(Nc1c(-c2ccncc2)ccc2c1CCC2)NS(=O)(=O)c1ccn(C2CCC2)n1 ; COc1cc(-c2ccc3c(c2NC(=O)NS(=O)(=O)c2nn(C4CC4)cc2F)CCC3)ccn1 ; COc1cc(-c2ccc3c(c2NC(=O)NS(=O)(=O)c2ccn(C(C)(C)CN4CC(F)(F)C4)n2)CCC3)ccn1]

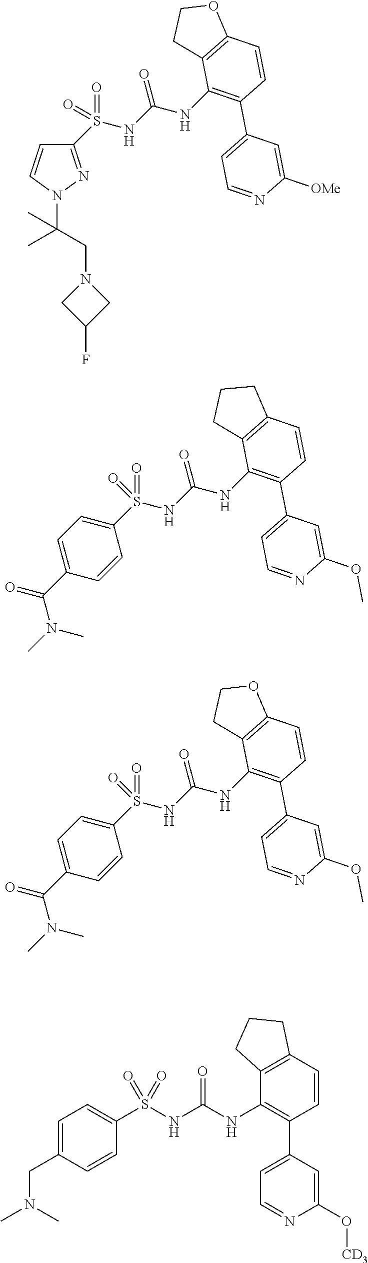

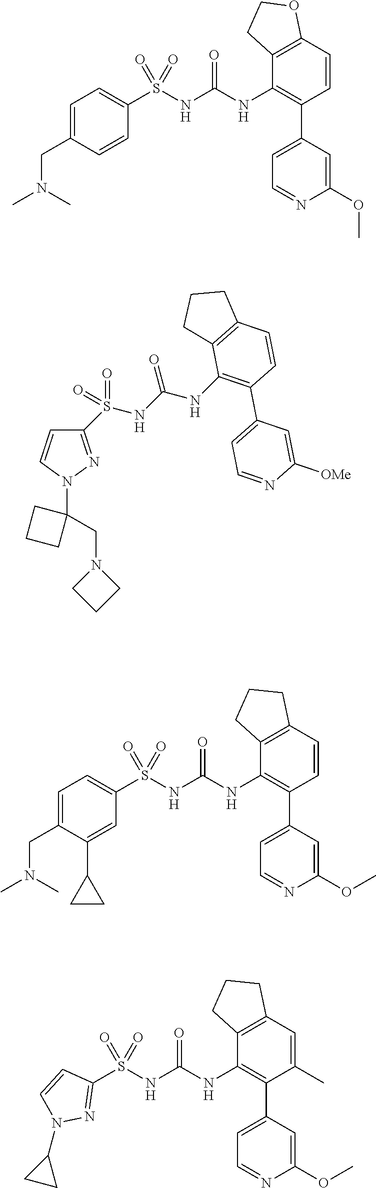

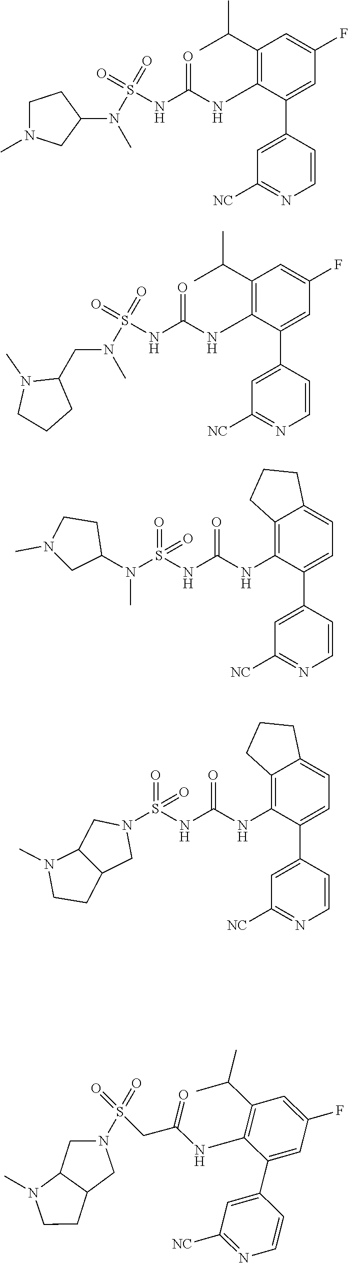

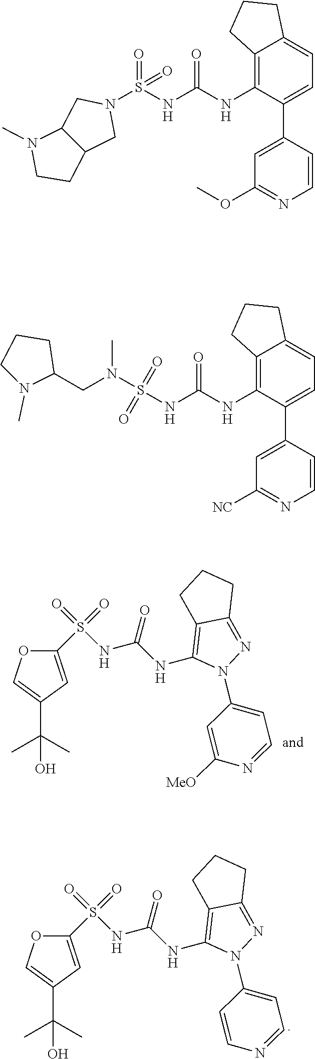 [SMILES: COc1cc(-c2ccc3c(c2NC(=O)NS(=O)(=O)N2CC4CCN(C)C4C2)CCC3)ccn1 ; CN1CCCC1CN(C)S(=O)(=O)NC(=O)Nc1c(-c2ccnc(C#N)c2)ccc2c1CCC2 ; COc1cc(-n2nc3c(c2NC(=O)NS(=O)(=O)c2cc(C(C)(C)O)co2)CCC3)ccn1 ; CC(C)(O)c1coc(S(=O)(=O)NC(=O)Nc2c3c(nn2-c2ccncc2)CCC3)c1]

11. (canceled)

12. A pharmaceutical composition comprising the compound or a pharmaceutically acceptable salt, solvate or prodrug thereof as claimed in claim 1, and a pharmaceutically acceptable excipient.

13. The pharmaceutical composition as claimed in claim 12, wherein the pharmaceutical composition is a topical pharmaceutical composition.

14. (canceled)

15. A method of treating or preventing a disease, disorder or condition in a subject, the method comprising the step of administering an effective amount of the compound or a pharmaceutically acceptable salt, solvate or prodrug thereof as claimed in claim 1 to the subject, thereby treating or preventing the disease, disorder or condition, optionally wherein the disease, disorder or condition is responsive to NLRP3 inhibition.

16. The method as claimed in claim 15, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia; and
(xvii) any disease where an individual has been determined to carry a germline or somatic non-silent mutation in NLRP3.

17. The method as claimed in claim 15, wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

18. (canceled)

19. The method as claimed in claim 15, wherein the compound is administered as a pharmaceutical composition further comprising a pharmaceutically acceptable excipient.

20. A method of inhibiting NLRP3 in a subject, comprising administering the compound or a pharmaceutically acceptable salt, solvate or prodrug thereof as claimed in claim 1 to the subject thereby inhibiting NLRP3.

21. A method of analysing inhibition of NLRP3 or an effect of inhibition of NLRP3 by a compound, comprising contacting a cell or non-human animal with the compound or a pharmaceutically acceptable salt, solvate or prodrug thereof as claimed in claim 1, and analysing inhibition of NLRP3 or an effect of inhibition of NLRP3 in the cell or non-human animal by the compound.
